(19) United States
(12) Patent Application Publication
(10) Pub. No.: US 2026/0250777 A1
(43) Pub. Date: Aug. 27, 2026

(54) METHODS FOR RNA EXTRACTION AND DETECTION FROM A URINE SAMPLE

(71) Applicant: Lynx Dx, Inc., Ann Arbor, MI (US)

(72) Inventor: Jacob MEYERS, Ann Arbor, MI (US)

(21) Appl. No.: 19/287,219

(22) Filed: Jul. 31, 2025

Related U.S. Application Data
(60) Provisional application No. 63/678,458, filed on Aug. 1, 2024.
(60) Provisional application No. 63/814,351, filed on May 29, 2025.

(51) Int. Cl. C12Q 1/6886; C12N 15/10; C12Q 1/24; C12Q 1/6806

(52) U.S. Cl. CPC C12Q 1/6886; C12N 15/1006; C12N 15/1096; C12Q 1/24; C12Q 1/6806; C12Q 2600/112; C12Q 2600/158; G01N 2333/92

(57) ABSTRACT

Provided herein are methods of RNA extraction from a urine sample, detection of cDNA reverse transcribed from extracted RNA and applications thereof (e.g., for the prognosis and/or diagnosis of disease, such as cancer).

### CROSS-REFERENCE TO RELATED APPLICATIONS

This application claims the benefit of and priority from U.S. Provisional Application No. 63/678,458, filed Aug. 1, 2024, and U.S. Provisional Application No. 63/814,351, filed May 29, 2025, the disclosure of each of which is incorporated by reference in its entirety.

### FIELD OF THE DISCLOSURE

Provided herein are methods for RNA extraction from a urine sample, detection of cDNA reverse transcribed from extracted RNA and applications thereof (e.g., for the prognosis and/or diagnosis of disease, such as cancer).

### REFERENCE TO AN ELECTRONIC SEQUENCE LISTING

The contents of the electronic sequence listing (LXDX_005_02US_SeqList_ST26.xml; Size: 529,382 bytes; and Date of Creation: Jul. 30, 2025) are herein incorporated by reference in its entirety.

### BACKGROUND

Liquid biopsy is a minimally invasive diagnostic approach that allows for the detection and analysis of biomarkers from bodily fluids, providing valuable information for disease diagnosis, prognosis, and/or treatment monitoring. Among various types of biological fluids, urine is an attractive specimen for liquid biopsy due to its ease of collection and non-invasive nature.

Current methods for RNA extraction from urine face several challenges that limit their efficiency and reliability. Urine contains low concentrations of RNA, which is often degraded or present in fragmented forms. Additionally, the complex composition of urine, which includes proteins and salts, can interfere with RNA extraction and subsequent analyses. Existing RNA extraction techniques often result in low yield and poor quality of RNA, which hampers the sensitivity and accuracy of downstream molecular assays, such as reverse transcription quantitative polymerase chain reaction (RT-qPCR) and next-generation sequencing (NGS). Currently, achieving detectable levels of RNA in urine samples from men for analysis related to prostate cancer, such as prognosis and/or diagnosis, requires an invasive and uncomfortable digital rectal examination (DRE) shortly before urine sample collection.

Thus, there exists a need for methods for improved extraction and detection of RNA from urine samples, particularly methods that provide an enhanced yield of extracted RNA while minimizing invasiveness and discomfort.

### SUMMARY OF THE INVENTION

Provided herein are methods for extracting RNA present in whole urine from a subject's urine sample, comprising:
admixing the whole urine and a first solid support to provide an RNA-bound first solid support, wherein the first solid support comprises silicon carbide;
admixing the RNA-bound first solid support and a polar organic solvent to provide an RNA-bound first solid support admixture and performing centrifugation of the RNA-bound first solid support admixture to provide a pellet and a supernatant;
removing the supernatant, and washing the pellet with a wash buffer to provide a washed RNA-bound first solid support;
admixing wash buffer, the washed RNA-bound first solid support, and a second solid support to provide an RNA-bound second solid support, wherein the second solid support comprises a silicon-based compound;
washing the RNA-bound second solid support to provide a washed RNA-bound second solid support;
treating the washed RNA-bound second solid support with deoxyribonuclease (DNase) to provide a DNase-treated second solid support and a flowthrough;
washing the DNase-treated second solid support to provide a washed DNase-treated second solid support;
drying the washed DNase-treated second solid support to provide a dried second solid support; and
eluting RNA from the dried second solid support to provide extracted RNA.

Also provided herein are compositions comprising whole urine and a solid support comprising silicon carbide.

Further provided herein are compositions comprising whole urine and an RNA-bound solid support.

Further provided herein are methods for making an RNA-bound solid support, comprising admixing whole urine and a solid support comprising silicon carbide to provide the RNA-bound solid support.

Further provided herein are methods for making a plurality of RNA-bound solid supports, comprising admixing whole urine and a plurality of solid supports comprising silicon carbide to provide the plurality of RNA-bound solid supports.

### BRIEF DESCRIPTION OF THE DRAWINGS

FIG. 1 is a graph showing the number of qPCR cycles (Crt) used to generate a detectable amount of cDNA reverse transcribed from KLK3 RNA extracted according to the method of Example 1a, as compared to cDNA reverse transcribed from RNA extracted using a Thermo Fisher RNA extraction method. S ####identifies individual samples from 24 men.

### DETAILED DESCRIPTION

Provided herein are methods for extracting RNA present in whole urine from a subject's urine sample, comprising:
admixing the whole urine and a first solid support to provide an RNA-bound first solid support, wherein the first solid support comprises silicon carbide;
admixing the RNA-bound first solid support and a polar organic solvent to provide an RNA-bound first solid support admixture and performing centrifugation of the RNA-bound first solid support admixture to provide a pellet and a supernatant;
removing the supernatant, and admixing the pellet and wash buffer to provide a washed RNA-bound first solid support;
admixing wash buffer, the washed RNA-bound first solid support, and a second solid support to provide an RNA-bound second solid support, wherein the second solid support comprises a silicon-based compound;
washing the RNA-bound second solid support to provide a washed RNA-bound second solid support;
treating the washed RNA-bound second solid support with DNase to provide a DNase-treated second solid support and a flowthrough;
washing the DNase-treated second solid support to provide a washed DNase-treated second solid support;
drying the washed DNase-treated second solid support to provide a dried second solid support; and
eluting RNA from the dried second solid support to provide extracted RNA.

The methods provided herein result in significantly improved RNA extraction and detection, e.g., by reverse transcription (RT) and qPCR, such that urine samples that would otherwise provide from standard methods (e.g., the Thermo Fisher method or Norgen method described herein) an amount of RNA that is insufficient for downstream molecular analyses can now provide an at least sufficient quantity of RNA for the same molecular analyses.

In some embodiments, extracted RNA is provided in an amount that is at least 3-fold greater than RNA obtained from a standard RNA extraction method.

### Definitions

To facilitate an understanding of the present disclosure, a number of terms and phrases are defined below:

As used herein, the term “subject” includes, but is not limited to, a mammal (e.g., a human, a non-human primate, a murine, a simian, an equine, a bovine, a porcine, a canine, a feline, and the like).

In some embodiments, the subject is a mammal. In some embodiments, the subject is a non-human primate. In some embodiments, the subject is a human subject. In some embodiments, the subject is female. In some embodiments, the subject is a male human subject who has a prostate.

As used herein, “GG≥2 prostate cancer” means “Grade Group 2 prostate cancer”. In some embodiments, the GG≥2 prostate cancer is GG≥3 prostate cancer. In some embodiments, the GG≥2 prostate cancer is GG≥4 prostate cancer. In some embodiments, the GG≥2 prostate cancer is GG5 prostate cancer.

As used herein, “GG<2 prostate cancer” means “Grade Group <2 prostate cancer”. In some embodiments, the GG<2 prostate cancer is GG 1 prostate cancer. In some embodiments, the GG<2 prostate cancer is no prostate cancer.

As used herein, the term “whole urine” refers to complete, undiluted output from the subject's urinary system. The whole urine used in the methods described herein is not further processed (e.g., fractionated by centrifugation or filtered to remove sediment or debris) from the subject's urine sample prior to admixing the whole urine with the first solid support.

As used herein, the term “first-catch urine” (also known as “first-void urine” or “first-pass urine”) refers to the first up to about 40 mL of urine passed by a subject on the day that the subject provides a urine sample.

As used herein, the term “about” means ±10% variation from an immediately following numerical value unless otherwise indicated. Where the term “about” is present immediately before a numerical value, the present disclosure also includes the specific numerical value itself, unless specifically stated otherwise.

As used herein, a “prostate biopsy-naïve” subject is a male human subject who has a prostate and who has not had a prostate biopsy prior to providing a urine sample useful in the present methods.

As used herein, a “prostate biopsy-prior negative” subject is a male human subject who has a prostate and who has had one or more prostate biopsies, none of which was positive for GG≥1 prostate cancer.

I. Extraction of RNA from Whole Urine

The methods described herein comprise extracting RNA present in whole urine from a subject's urine sample. In some embodiments, the whole urine comprises first-catch urine. In some embodiments, the whole urine is first-catch urine.

In some embodiments, the whole urine is decanted or otherwise removed, e.g., by pipetting, from the urine sample. In some embodiments, the whole urine has a volume of about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, or about 15 mL. In some embodiments, the whole urine has a volume of about 5 mL to about 10 mL. In some embodiments, the whole urine has a volume of about 5 mL. In some embodiments, the methods comprise admixing the whole urine and the first solid support to provide the RNA-bound first solid support. In some embodiments, the first solid support is one of a plurality of first solid supports, the RNA-bound first solid support is one of a plurality of RNA-bound first solid supports, and the methods comprise admixing the whole urine and the plurality of first solid supports to provide the plurality of RNA-bound first solid supports. In some embodiments, the methods comprise admixing whole urine, a preservative and the first solid support to provide the RNA-bound first solid support. In some embodiments, the methods comprise admixing the whole urine, a preservative and a plurality of first solid supports to provide a plurality of RNA-bound first solid supports. In some embodiments, the preservative is Urine Conservation Medium (UCM). In some embodiments, the preservative is Urinary Analyte Stabilizer (UAS).

In some embodiments, the methods described herein do not comprise isolating an exosome, e.g., an exosome comprising an RNA, from the whole urine or the subject's urine sample. Without being bound by theory, it is believed that avoiding an exosome-isolation step makes the sample processing more robust (e.g., less likely to fail) and obviates a requirement to preserve exosomes in the urine prior to RNA extraction, both streamlining sample processing and reducing processing cost per sample.

In some embodiments, admixing the whole urine and the first solid support comprises adding the whole urine to the first solid support, or vice versa, e.g., by pipetting. In some embodiments, admixing the whole urine and the plurality of first solid supports comprises adding the whole urine to the plurality of first solid supports, or vice versa, e.g., by pipetting. In some embodiments, the first solid support comprises a silicon carbide surface for interaction with RNA, which can be bound to the silicon carbide surface by, e.g., adsorption.

In some embodiments, the first solid support is silicon carbide. In some embodiments, the first solid support is a silicon carbide particle. In some embodiments, the silicon carbide particle is a silicon carbide nanoparticle. In some embodiments, the silicon carbide particle is one of a plurality of silicon carbide particles, and the methods comprise admixing the whole urine and the plurality of silicon carbide particles. In some embodiments, the silicon carbide nanoparticle is one of a plurality of silicon carbide nanoparticles, and the methods comprise admixing the whole urine and the plurality of silicon carbide particles. In some embodiments, the plurality of silicon carbide particles, e.g., silicon carbide nanoparticles, are present in a slurry, and the methods comprise admixing the whole urine and the slurry. In some embodiments, the plurality of silicon carbide particles is Slurry C3 commercially available from Norgen Biotek Corp. In some embodiments, the methods comprise admixing about 0.07 mL of slurry for every 1 mL of whole urine. In some embodiments, the methods comprise admixing about 0.35 mL of slurry for every 5 mL of whole urine. In some embodiments, the methods comprise admixing the whole urine and the slurry at a ratio of about 1 to about 0.05 (v/v) whole urine to slurry, about 1 to about 0.06 (v/v) whole urine to slurry, about 1 to about 0.07 (v/v) whole urine to slurry, about 1 to about 0.08 (v/v) whole urine to slurry, about 1 to about 0.09 (v/v) whole urine to slurry, or about 1 to about 0.1 (v/v) whole urine to slurry. In some embodiments, the admixing the whole urine and the slurry occurs at a concentration of about 1 to about 0.07 (v/v) whole urine to slurry.

In some embodiments, the first solid support is one of a plurality of silicon carbide particles, and the methods comprise admixing about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg of the plurality of silicon carbide particles per mL of whole urine. In some embodiments, the methods comprise admixing about 100 mg of the plurality of silicon carbide particles per mL of whole urine. In some embodiments, the plurality of silicon carbide particles is present in a slurry.

In some embodiments, the first solid support is contained in a column, and the methods comprise admixing within the column the whole urine and the first solid support. In some embodiments, the plurality of first solid supports is contained in a column, and the methods comprise admixing within the column the whole urine and the plurality of first solid supports. In some embodiments, the plurality of first solid supports is a plurality of silicon carbide particles, and the column contains the plurality of silicon carbide particles. In some embodiments, the plurality of silicon carbide particles is present in a slurry.

In some embodiments, the methods comprise admixing the whole urine, the first solid support and a lysis buffer. In some embodiments, the methods comprise admixing the whole urine, the plurality of first solid supports and a lysis buffer. In some embodiments, the plurality of first solid supports, e.g., silicon carbide particles, is present in a slurry.

In some embodiments, the methods comprise admixing whole urine, a preservative, a lysis buffer, and the first solid support to provide the RNA-bound first solid support. In some embodiments, the methods comprise admixing whole urine, a preservative, a lysis buffer, and a plurality of first solid supports to provide a plurality of RNA-bound first solid supports. In some embodiments, the preservative is UCM. In some embodiments, the preservative is UAS.

Lysis buffers suitable for the methods described herein include, for example, and without limitation, nonionic and ionic detergents or surfactants. Illustrative nonionic surfactants include, but are not limited to, t-octylphenoxypolyethoxyethanol (e.g., TRITON X-100), (octylphenoxy)polyethoxyethanol (e.g., IGEPALCA-630/NP-40), triethyleneglycol monolauryl ether (e.g., BRIJ 30), sorbitan monolaurate (e.g., SPAN 20), or the polysorbate family of chemicals, such as polysorbate 20 (e.g., TWEEN 20). Other commercially available Polysorbates include TWEEN 40, TWEEN 60 and TWEEN 80 (Sigma-Aldrich, St. Louis, MO). Ionic detergents, such as sodium dodecyl sulfate (SDS) can be used in sample preparations for nucleic acid. The lysis buffer can optionally comprise a chaotropic salt, such as guanidinium thiocyanate or guanidinium chloride. In some embodiments, the lysis buffer comprises phenol. In some embodiments, the lysis buffer does not comprise phenol. In some embodiments, the lysis buffer comprises guanidinium thiocyanate. In some embodiments, the lysis buffer comprises guanidinium thiocyanate and β-mercaptoethanol. In some embodiments, the lysis buffer comprises guanidinium chloride. In some embodiments, the lysis buffer comprises guanidinium chloride and β-mercaptoethanol. In some embodiments, the lysis buffer is a phenol-based lysis buffer.

In some embodiments, the lysis buffer comprises a reducing agent, e.g., 2-aminoethanethiol, tris-carboxyethylphosphine (TCEP), or β-mercaptoethanol. In some embodiments, the lysis buffer comprises β-mercaptoethanol. In some embodiments, the β-mercaptoethanol is present in the lysis buffer at a concentration of up to about 5% by volume of the lysis buffer. In some embodiments, the lysis buffer comprises β-mercaptoethanol at a concentration of about 1% to about 2% by volume of the lysis buffer. In some embodiments, the β-mercaptoethanol is present in the lysis buffer at a concentration of about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, or about 5% by volume of the lysis buffer. In some embodiments, the β-mercaptoethanol is present in the lysis buffer at a concentration of about 1% by volume of the lysis buffer. In some embodiments, the subject did not have a digital rectal examination (DRE) within about 180 minutes before providing the urine sample.

In some embodiments, the methods comprise admixing the RNA-bound first solid support and a polar organic solvent to provide an RNA-bound first solid support admixture. In some embodiments, the RNA-bound first solid support is a plurality of RNA-bound first solid supports, and the methods comprise admixing the plurality of RNA-bound first solid supports and a polar organic solvent to provide an RNA-bound first solid support admixture. In some embodiments, the polar organic solvent is acetone, acetonitrile, dimethylformamide, dimethylsulfoxide, ethyl acetate, ethylene glycol or a C₁-C₄ alcohol. In some embodiments, the polar organic solvent is a C₁-C₄ alcohol, and the C₁-C₄ alcohol is methanol, ethanol, n-propanol, iso-propanol, n-butanol, iso-butanol, sec-butanol or tert-butanol. In some embodiments, the polar organic solvent is a C₁-C₄ alcohol, the C₁-C₄ alcohol is ethanol and the ethanol is 190 proof ethanol, ≥95.0% ethanol, ≥99.5% ethanol, ≥99.8% ethanol, ≥99.9% ethanol or 100% ethanol. In some embodiments, the polar organic solvent is 100% ethanol. In some embodiments, the volume of the polar organic solvent, e.g., ethanol, is about 1 mL, about 2 mL, about 3 mL, about 4 mL, about 5 mL, about 6 mL, about 7 mL, about 8 mL, about 9 mL, or about 10 mL. In some embodiments, the admixing the RNA-bound first solid support and a polar organic solvent comprises admixing, e.g., vortexing, the RNA-bound first solid support and the polar organic solvent, or admixing, e.g., vortexing, the plurality of RNA-bound first solid supports and polar organic solvent, for at least about 5 seconds, at least about 10 seconds, at least about 15 seconds, at least about 20 seconds, at least about 25 seconds, or at least about 30 seconds. In some embodiments, the admixing the RNA-bound first solid support and a polar organic solvent comprises admixing, e.g., vortexing, the RNA-bound first solid support and the polar organic solvent, or admixing, e.g., vortexing, the plurality of RNA-bound first solid supports and polar organic solvent, for about 10 seconds.

In some embodiments, the methods comprise performing a centrifugation of the RNA-bound first solid support admixture to provide a pellet and a supernatant. In some embodiments, the RNA-bound first solid support admixture comprises an RNA-bound first solid support. In some embodiments, the RNA-bound first solid support admixture comprises a plurality of RNA-bound first solid supports.

In some embodiments, the performing the centrifugation of the RNA-bound first solid support admixture is for about 1 minute, about 2 minutes, about 3 minutes, about 4 minutes, about 5 minutes, about 6 minutes, about 7 minutes, about 8 minutes, about 9 minutes, about 10 minutes, or more. In some embodiments, the performing the centrifugation of the RNA-bound first solid support admixture is for about 5 minutes.

In some embodiments, the performing the centrifugation of the RNA-bound first solid support admixture comprises subjecting the RNA-bound first solid support admixture to a centrifugal force of from about 500×g to about 5,000×g, from about 1,500×g to about 3,000×g, or from about 1,500×g to about 2,500×g. In some embodiments, the performing the centrifugation of the RNA-bound first solid support admixture comprises subjecting the RNA-bound first solid support admixture to a centrifugal force of about 1,000×g, about 2,000×g, about 3,000×g, 4,000×g, about 5,000×g, or more. In some embodiments, the performing the centrifugation of the RNA-bound first solid support admixture comprises subjecting the RNA-bound first solid support admixture to a centrifugal force of about 2,000×g.

In some embodiments, the performing the centrifugation of the RNA-bound first solid support admixture occurs at a temperature of about 20° C. to about 30° C. In some embodiments, the performing the centrifugation of the RNA-bound first solid support admixture occurs at a temperature of about 20° C., about 21° C., about 22° C., about 23° C., about 24° C., about 25° C., about 26° C., about 27° C., about 28° C., about 29° C., or about 30° C. In some embodiments, the performing the centrifugation of the RNA-bound first solid support admixture occurs at a temperature of about 23° C. to about 30° C. In some embodiments, the performing the centrifugation of the RNA-bound first solid support admixture occurs at a temperature of about 23° C. In some embodiments, the performing the centrifugation of the RNA-bound first solid support admixture occurs at a temperature of about 30° C.

In some embodiments, the supernatant is removed and optionally discarded. In some embodiments, the supernatant is removed by decanting. In some embodiments, the supernatant is removed by pipetting.

In some embodiments, the methods comprise removing the supernatant, and washing the pellet with a wash buffer to provide a washed RNA-bound first solid support. In some embodiments, the washed RNA-bound first solid support is one of a plurality of washed RNA-bound first solid supports, and the methods comprise removing the supernatant, and admixing the pellet and a wash buffer to provide the plurality of washed RNA-bound first solid supports. In some embodiments, the wash buffer has a volume of about 50 μL, about 100 μL, about 150 μL, about 200 μL, about 250 μL, about 300 μL, about 350 μL, about 400 μL, about 450 μL, about 500 μL, about 550 μL, about 600 μL, about 650 μL, about 700 μL, about 750 μL, about 800 μL, about 850 μL, about 900 μL, about 950 μL, about 1000 μL, or more. In some embodiments, the wash buffer has a volume of about 500 μL. In some embodiments, the washing the pellet with the wash buffer comprises pipetting or vortexing one or both of the pellet and wash buffer. In some embodiments, the methods comprise discarding the supernatant after removing it.

In some embodiments, the methods comprise admixing a wash buffer, the washed RNA-bound first solid support, and a second solid support to provide an RNA-bound second solid support, wherein the second solid support comprises a silicon-based compound. Silicon-based compounds include, but are not limited to, silicon carbide, silica, polysilicic materials, silicates, borosilicates, inorganic glasses, and silica materials such as silica particles, silica fibers, glass fibers, glass particles, glass powders, silica sand, silica gel, diatomaceous earth, glass, alkylsilica, aluminosilicate, and borosilicate and the like. The silicon-based compound can be present in a slurry, can be in a bead-based format, or can be incorporated in one or more other structures, such as silica membranes, silica impregnated or coated filters, or silica coated magnetic beads. The second solid support described herein can be porous or non-porous, permeable or impermeable, including but not limited to, and in the form of, present in or contained in a membrane, column, slurry, resin, filter paper, sheet, particle, magnetic particle, bead, magnetic bead, gel, powder, fiber, and the like. In some embodiments, the second solid support comprises a silica surface for interaction with RNA, which can be bound to the silica surface by, e.g., adsorption. In some embodiments, the washed RNA-bound first solid support is one of a plurality of washed RNA-bound first solid supports, the second solid support is one of a plurality of second solid supports, the RNA bound second solid support is a plurality of RNA-bound second solid supports, and the methods comprise admixing a wash buffer, the plurality of washed RNA-bound first solid supports, and a plurality of second solid supports to provide a plurality of RNA-bound second solid supports.

In some embodiments, the second solid support is silica. In some embodiments, the second solid support is a silica particle. In some embodiments, the silica particle is one of a plurality of silica particles. In some embodiments, the plurality of silica particles is present in a slurry. In some embodiments, the plurality of silica particles is contained in a column.

In some embodiments, the second solid support is silicon carbide. In some embodiments, the second solid support is a silicon carbide particle. In some embodiments, the silicon carbide particle is one of a plurality of silicon carbide particles. In some embodiments, the plurality of silicon carbide particles is present in a slurry. In some embodiments, the plurality of silicon carbide particles is contained in a column.

In some embodiments, the second solid support comprises a first silicon-based compound, wherein the first silicon-based compound is silica, and further comprises a second silicon-based compound, wherein the second silicon-based compound is silicon carbide.

In some embodiments, the second solid support is one of a plurality of silica particles. In some embodiments, the second solid support is one of a plurality of silicon carbide particles.

In some embodiments, the washed RNA-bound first solid support is one of a plurality of washed RNA-bound first solid supports, the second solid support is one of a plurality of silica particles, and the methods comprise admixing a wash buffer, the plurality of washed RNA-bound first solid supports, and the plurality of silica particles to provide a plurality of RNA-bound second solid supports.

In some embodiments, the washed RNA-bound first solid support is one of a plurality of washed RNA-bound first solid supports, the second solid support is one of a plurality of silicon carbide particles, and the methods comprise admixing a wash buffer, the plurality of washed RNA-bound first solid supports, and the plurality of silicon carbide particles to provide a plurality of RNA-bound second solid supports.

In some embodiments, the methods comprise admixing a wash buffer, the plurality of washed RNA-bound first solid supports, the plurality of silica particles and a plurality of silicon carbide particles to provide a plurality of RNA-bound second solid supports.

In some embodiments, the methods comprise admixing a wash buffer, a washed RNA-bound first solid support, a second solid support and a third solid support to provide an RNA-bound second solid support and an RNA-bound third solid support, wherein the second solid support comprises a silicon-based compound that is silica, and wherein the third solid support comprises a silicon-based compound that is silicon carbide.

In some embodiments, the washed RNA-bound first solid support is one of a plurality of washed RNA-bound first solid supports, the second solid support is one of a plurality of second solid supports, the third solid support is one of a plurality of third solid supports, and the methods comprise admixing the wash buffer, the plurality of washed RNA-bound first solid supports, the plurality of second solid supports and the plurality of third solid support to provide a plurality of RNA-bound second solid supports and a plurality of RNA-bound third solid supports, wherein each second solid support comprises a silicon-based compound that is silica, and wherein each third solid support comprises a silicon-based compound that is silicon carbide.

In some embodiments, the plurality of second solid supports and plurality of third solid supports are present in a slurry. In some embodiments, the plurality of second solid supports and plurality of third solid supports are present in a slurry, wherein each second solid support comprises a silicon-based compound that is silica, and wherein each third solid support comprises a silicon-based compound that is silicon carbide.

In some embodiments, one or both of the second solid support and third solid support are contained in a filter, e.g., a membrane filter. In some embodiments, one or both of (i) the plurality of second solid supports and (ii) the plurality of third solid supports are contained in a filter, e.g., a membrane filter. In some embodiments, the membrane of the membrane filter is a positively charged membrane. In some embodiments, the membrane comprises cellulose. In some embodiments, the membrane comprises polyethersulfone (PES). In some embodiments, the filter is a component of a filter plate, e.g., a 96-well filter plate. In some embodiments, the filter is a component of a column, e.g., a spin column.

In some embodiments, one or both of the second solid support and third solid support are contained in a column. In some embodiments, one or both of (i) the plurality of second solid supports and (ii) the plurality of third solid supports are contained in a column. In some embodiments, the plurality of second solid supports and plurality of third solid supports are contained in a column, wherein each second solid support comprises a silicon-based compound that is silica, and wherein each third solid support comprises a silicon-based compound that is silicon carbide. In some embodiments, the plurality of second solid supports and plurality of third solid supports are contained in a column, wherein the plurality of second solid supports is a plurality of silica particles, and wherein the plurality of third solid supports is a plurality of silicon carbide particles. Examples of columns containing a plurality of silica particles and a plurality of silicon carbide particles and useful in the present methods are disclosed in U.S. Pat. No. 9,845,463 incorporated by reference herein, for example, at column 4, lines 51-67; columns 5-9; and column 10, lines 1-42; and 9,422,596, incorporated by reference herein, for example at column 4, lines 43-67; columns 5-9; and column 10, lines 1-26.

In some embodiments, the column comprises a first portion and a second portion, wherein the first portion comprises the plurality of second solid supports, the second portion comprises the plurality of third solid supports, each second solid support comprises a silicon-based compound that is silica and each third solid support comprises a silicon-based compound that is silicon carbide. In some embodiments, the first portion is a first layer, and the second portion is a second layer.

In some embodiments, the third solid support comprises a silicon-based compound that is silicon carbide. In some embodiments, the silicon carbide is a plurality of silicon carbide particles and the amount of the plurality of silicon carbide particles is about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg. In some embodiments, the amount of the plurality of silicon carbide particles is about 100 mg. In some embodiments, the plurality of silicon carbide particles is contained in a column.

In some embodiments, the column contains a filter. In some embodiments, the filter comprises or is a membrane. In some embodiments, one or both of the filter and membrane contain the plurality of second solid supports or the plurality of third solid supports. In some embodiments, one or both of the filter and membrane contain the plurality of second solid supports and the plurality of third solid supports. In some embodiments, the filter or membrane has a thickness of at least 0.5 mm. In some embodiments, the filter or membrane has a pore size of <about 0.5 μm, in some embodiments, ≤about 0.22 μm. In some embodiments, the column is a well of a filter plate.

In some embodiments, the admixing the wash buffer, the washed RNA-bound first solid support, and the second solid support comprises performing a centrifugation of the wash buffer, the washed RNA-bound first solid support, and the second solid support to provide the RNA-bound second solid support and a flowthrough. In some embodiments, the admixing the wash buffer, the washed RNA-bound first solid support, the second solid support and the third solid support comprises performing a centrifugation of the wash buffer, the washed RNA-bound first solid support, the second solid support and the third to provide the RNA-bound second solid support, an RNA-bound third solid support and a flowthrough. In some embodiments, the flowthrough is removed and optionally discarded following the centrifugation of the wash buffer, the washed RNA-bound first solid support, the second solid support and optionally the third solid support.

In some embodiments, the performing the centrifugation of the wash buffer, the washed RNA-bound first solid support, the second solid support and optionally the third solid support comprises subjecting the wash buffer, the washed RNA-bound first solid support, the second solid support and optionally the third solid support to a centrifugal force of from about 500×g to about 5,000×g, from about 2,000×g to about 4,000×g, or about 2,500×g to about 3,500×g. In some embodiments, the performing the centrifugation of the wash buffer, the washed RNA-bound first solid support, the second solid support and optionally the third solid support comprises subjecting the wash buffer, the washed RNA-bound first solid support, the second solid support and optionally the third solid support to a centrifugal force of about 1,000×g, about 2,000×g, about 3,000×g, 4,000×g, about 5,000×g, or more. In some embodiments, the performing the centrifugation of the wash buffer, the washed RNA-bound first solid support, the second solid support and optionally the third solid support comprises subjecting the wash buffer, the washed RNA-bound first solid support, the second solid support and optionally the third solid support to a centrifugal force of about 3,146×g. In some embodiments, the washed RNA-bound first solid support is one of a plurality of washed RNA-bound first solid supports, the second solid support is one of a plurality of second solid supports, and the methods comprise performing a centrifugation of the wash buffer, the plurality of washed RNA-bound first solid supports, and plurality of the second solid supports at a centrifugal force of about 3,000×g or about 3,146×g. In some embodiments, the washed RNA-bound first solid support is one of a plurality of washed RNA-bound first solid supports, the second solid support is one of a plurality of second solid supports, the third solid support is one of a plurality of third solid supports, and the methods comprise performing a centrifugation of the wash buffer, the plurality of washed RNA-bound first solid supports, plurality of the second solid supports and the plurality of third solid supports at a centrifugal force of about 3,000×g or about 3,146×g.

In some embodiments, the performing the centrifugation of the wash buffer, the washed RNA-bound first solid support, the second solid support and optionally the third solid support is for about 1 minute, about 2 minutes, about 3 minutes, about 4 minutes, about 5 minutes, about 6 minutes, about 7 minutes, about 8 minutes, about 9 minutes, about 10 minutes, or more. In some embodiments, the performing the centrifugation of the wash buffer, the washed RNA-bound first solid support, and the second solid support and optionally the third solid support is for about 2 minutes. In some embodiments, the performing the centrifugation of the wash buffer, the plurality of washed RNA-bound first solid supports, the plurality of second solid supports and the plurality of third solid supports is for about 1 minute, about 2 minutes, about 3 minutes, about 4 minutes, about 5 minutes, about 6 minutes, about 7 minutes, about 8 minutes, about 9 minutes, about 10 minutes, or more. In some embodiments, the performing the centrifugation of the wash buffer, the plurality of washed RNA-bound first solid supports, the plurality of second solid supports and the plurality of third solid supports is for about 2 minutes. In some embodiments, the performing the centrifugation of the wash buffer, the washed RNA-bound first solid support, the second solid support and optionally the third solid support occurs at a temperature of about 20° C. to about 30° C. In some embodiments, the performing the centrifugation of the wash buffer, the washed RNA-bound first solid support, the second solid support and optionally the third solid support occurs at a temperature of about 20° C., about 21° C., about 22° C., about 23° C., about 24° C., about 25° C., about 26° C., about 27° C., about 28° C., about 29° C., or about 30° C. In some embodiments, the performing the centrifugation of the wash buffer, the washed RNA-bound first solid support, the second solid support and optionally the third solid support occurs at a temperature of about 23° C. to about 30° C. In some embodiments, the performing the centrifugation of the wash buffer, the washed RNA-bound first solid support, the second solid support and optionally the third solid support occurs at a temperature of about 23° C. In some embodiments, the performing the centrifugation of the wash buffer, the washed RNA-bound first solid support, the second solid support and optionally the third solid support occurs at a temperature of about 30° C. In some embodiments, the washed RNA-bound first solid support is one of a plurality of washed RNA-bound first solid supports, the second solid support is one of a plurality of second solid supports, and the methods comprise performing a centrifugation of the wash buffer, the plurality of washed RNA-bound first solid supports, and plurality of the second solid supports at a temperature of about 23° C. to about 30° C. In some embodiments, the methods comprise performing a centrifugation of the wash buffer, the plurality of washed RNA-bound first solid supports, and plurality of the second solid supports at a temperature of about 23° C. In some embodiments, the methods comprise performing a centrifugation of the wash buffer, the plurality of washed RNA-bound first solid supports, and plurality of the second solid supports at a temperature of about 30° C. In some embodiments, the washed RNA-bound first solid support is one of a plurality of washed RNA-bound first solid supports, the second solid support is one of a plurality of second solid supports, the third solid support is one of a plurality of third solid supports, and the methods comprise performing a centrifugation of the wash buffer, the plurality of washed RNA-bound first solid supports, the plurality of the second solid supports and the plurality of third solid supports at a temperature of about 23° C. to about 30° C. In some embodiments, the methods comprise performing a centrifugation of the wash buffer, the plurality of washed RNA-bound first solid supports, the plurality of the second solid supports and the plurality of third solid supports at a temperature of about 23° C. In some embodiments, the methods comprise performing a centrifugation of the wash buffer, the plurality of washed RNA-bound first solid supports, the plurality of the second solid supports and the plurality of third solid supports at a temperature of about 30° C.

In some embodiments, the methods comprise washing the RNA-bound second solid support to provide a washed RNA-bound second solid support. In some embodiments, the method comprises washing the RNA-bound second solid support and RNA-bound third solid support to provide a washed RNA-bound second solid support and a washed RNA-bound third solid support. In some embodiments, the washing the RNA-bound second solid support comprises admixing the RNA-bound second solid support and a wash buffer to provide an RNA-bound second solid support and wash buffer admixture, and performing a centrifugation of the RNA-bound second solid support and wash buffer admixture to provide the washed RNA-bound second solid support and a flowthrough. In some embodiments, the washing the RNA-bound second solid support and RNA-bound third solid support comprises admixing the RNA-bound second solid support, the RNA-bound third solid support and a wash buffer to provide an RNA-bound second solid support, RNA-bound third solid support and wash buffer admixture, and performing a centrifugation of the RNA-bound second solid support, RNA-bound third solid support and wash buffer admixture to provide the washed RNA-bound second solid support, the washed RNA-bound third solid support and a flowthrough. In some embodiments, the flowthrough is discarded. In some embodiments, the wash buffer has a volume of about 50 μL, about 100 μL, about 150 μL, about 200 μL, about 250 μL, about 300 μL, about 350 μL, about 400 μL, about 450 μL, about 500 μL, about 550 μL, about 600 μL, about 650 μL, about 700 μL, about 750 μL, about 800 μL, about 850 μL, about 900 μL, about 950 μL, about 1000 μL, or more. In some embodiments, the wash buffer has a volume of about 400 μL. In some embodiments, the washed RNA-bound second solid support is one of a plurality of washed RNA-bound second solid supports. In some embodiments, the methods comprise washing a plurality of RNA-bound second solid supports to provide a plurality of washed RNA-bound second solid supports. In some embodiments, the washed RNA-bound second solid support is one of a plurality of washed RNA-bound second solid supports, and the RNA-bound third solid support is one of a plurality of RNA-bound third solid supports. In some embodiments, the methods comprise washing the plurality of RNA-bound second solid supports and the plurality of RNA-bound third solid supports to provide a plurality of washed RNA-bound second solid supports and a plurality of washed RNA-bound third solid supports.

In some embodiments, the performing the centrifugation of the RNA-bound second solid support and the wash buffer admixture comprises subjecting the RNA-bound second solid support and the wash buffer admixture to a centrifugal force of from about 500×g to about 5,000×g, from about 2,000×g to about 4,000×g, or about 2,500×g to about 3,500×g. In some embodiments, the performing the centrifugation of the RNA-bound second solid support and the wash buffer admixture comprises subjecting the RNA-bound second solid support and the wash buffer admixture to a centrifugal force of about 1,000×g, about 2,000×g, about 3,000×g, 4,000×g, about 5,000×g, or more. In some embodiments, the performing the centrifugation of the RNA-bound second solid support and the wash buffer admixture comprises subjecting the RNA-bound second solid support and the wash buffer admixture to a centrifugal force of about 3,146×g. In some embodiments, the RNA-bound second solid support is one of a plurality of RNA-bound second solid supports, and the methods comprise performing a centrifugation of the plurality of RNA-bound second solid supports and the wash buffer admixture at a centrifugal force of about 3,000×g or about 3,146×g.

In some embodiments, the performing the centrifugation of the RNA-bound second solid support, the RNA-bound third solid support and the wash buffer admixture comprises subjecting the RNA-bound second solid support, the RNA-bound third solid support and the wash buffer admixture to a centrifugal force of from about 500×g to about 5,000×g, from about 2,000×g to about 4,000×g, or about 2,500×g to about 3,500×g. In some embodiments, the performing the centrifugation of the RNA-bound second solid support, the RNA-bound third solid support and the wash buffer admixture comprises subjecting the RNA-bound second solid support, the RNA-bound third solid support and the wash buffer admixture to a centrifugal force of about 1,000×g, about 2,000×g, about 3,000×g, 4,000×g, about 5,000×g, or more. In some embodiments, the performing the centrifugation of the RNA-bound second solid support, the RNA-bound third solid support and the wash buffer admixture comprises subjecting the RNA-bound second solid support, the RNA-bound third solid support and the wash buffer admixture to a centrifugal force of about 3,146×g. In some embodiments, the RNA-bound second solid support is one of a plurality of RNA-bound second solid supports, the RNA-bound third solid support is one of a plurality of RNA-bound third solid supports, and the methods comprise performing a centrifugation of the plurality of RNA-bound second solid supports, the RNA-bound third solid supports and the wash buffer admixture at a centrifugal force of about 3,000×g or about 3,146×g.

In some embodiments, the performing the centrifugation of the RNA-bound second solid support and the wash buffer admixture occurs at a temperature of about 20° C. to about 30° C. In some embodiments, the performing the centrifugation of the RNA-bound second solid support and the wash buffer admixture occurs at a temperature of about 20° C., about 21° C., about 22° C., about 23° C., about 24° C., about 25° C., about 26° C., about 27° C., about 28° C., about 29° C., or about 30° C. In some embodiments, the performing the centrifugation of the RNA-bound second solid support and the wash buffer admixture occurs at a temperature of about 23° C. to about 30° C. In some embodiments, the performing the centrifugation of the RNA-bound second solid support and the wash buffer admixture occurs at a temperature of about 23° C. In some embodiments, the performing the centrifugation of the RNA-bound second solid support and the wash buffer admixture occurs at a temperature of about 30° C. In some embodiments, the RNA-bound second solid support is one of a plurality of RNA-bound second solid supports, and the methods comprise performing a centrifugation of the plurality of RNA-bound second solid supports and the wash buffer admixture at a temperature of about 23° C. to about 30° C. In some embodiments, the methods comprise performing a centrifugation of the plurality of RNA-bound second solid supports and the wash buffer admixture at a temperature of about 23° C. In some embodiments, the methods comprise performing a centrifugation of the plurality of RNA-bound second solid supports and the wash buffer admixture at a temperature of about 30° C.

In some embodiments, the performing the centrifugation of the RNA-bound second solid support, the RNA-bound third solid support and the wash buffer admixture occurs at a temperature of about 20° C. to about 30° C. In some embodiments, the performing the centrifugation of the RNA-bound second solid support, the RNA-bound third solid support and the wash buffer admixture occurs at a temperature of about 20° C., about 21° C., about 22° C., about 23° C., about 24° C., about 25° C., about 26° C., about 27° C., about 28° C., about 29° C., or about 30° C. In some embodiments, the performing the centrifugation of the RNA-bound second solid support, the RNA-bound third solid support and the wash buffer admixture occurs at a temperature of about 23° C. to about 30° C. In some embodiments, the performing the centrifugation of the RNA-bound second solid support, the RNA-bound third solid support and the wash buffer admixture occurs at a temperature of about 23° C. In some embodiments, the performing the centrifugation of the RNA-bound second solid support, the RNA-bound third solid support and the wash buffer admixture occurs at a temperature of about 30° C. In some embodiments, the RNA-bound second solid support is one of a plurality of RNA-bound second solid supports, the RNA-bound third solid support is one of a plurality of RNA-bound third solid supports, and the methods comprise performing a centrifugation of the plurality of RNA-bound second solid supports, the plurality of RNA-bound third solid supports and the wash buffer admixture at a temperature of about 23° C. to about 30° C. In some embodiments, the methods comprise performing a centrifugation of the plurality of RNA-bound second solid supports and the wash buffer admixture at a temperature of about 23° C. In some embodiments, the methods comprise performing a centrifugation of the plurality of RNA-bound second solid supports, the plurality of RNA-bound third solid supports and the wash buffer admixture at a temperature of about 30° C.

In some embodiments, the performing the centrifugation of the RNA-bound second solid support and the wash buffer admixture is for about 1 minute, about 2 minutes, about 3 minutes, about 4 minutes, about 5 minutes, about 6 minutes, about 7 minutes, about 8 minutes, about 9 minutes, about 10 minutes, or more. In some embodiments, the performing the centrifugation of the RNA-bound second solid support and the wash buffer admixture is for about 2 minutes.

In some embodiments, the performing the centrifugation of the RNA-bound second solid support, the RNA-bound third solid support and the wash buffer admixture is for about 1 minute, about 2 minutes, about 3 minutes, about 4 minutes, about 5 minutes, about 6 minutes, about 7 minutes, about 8 minutes, about 9 minutes, about 10 minutes, or more. In some embodiments, the performing the centrifugation of the RNA-bound second solid support, the RNA-bound third solid support and the wash buffer admixture is for about 2 minutes.

In some embodiments, the methods comprise treating the washed RNA-bound second solid support with DNase to provide a DNase-treated second solid support and a flowthrough. In some embodiments, the method further comprises treating the washed RNA-bound second solid support and washed RNA-bound third solid support with deoxyribonuclease (DNase) to provide a DNase-treated second solid support, a DNase-treated third solid support and a flowthrough. In some embodiments, the treating comprises performing a centrifugation of the washed RNA-bound second solid support and DNase to provide the DNase-treated second solid support and the flowthrough. In some embodiments, the treating comprises performing a centrifugation of the washed RNA-bound second solid support, the washed RNA-bound third solid support and DNase to provide the DNase-treated second solid support, the DNase-treated third solid support and the flowthrough. In some embodiments, the washed RNA-bound second solid support is one of a plurality of washed RNA-bound second solid supports, the DNase-treated second solid support is one of a plurality of DNase-treated second solid supports, and the treating comprises performing a centrifugation of the plurality of washed RNA-bound second solid supports and DNase to provide the plurality of DNase-treated second solid supports and the flowthrough. In some embodiments, the washed RNA-bound second solid support is one of a plurality of washed RNA-bound second solid supports, the washed RNA-bound third solid support is one of a plurality of washed RNA-bound third solid supports, and the treating comprises performing a centrifugation of the plurality of washed RNA-bound second solid supports, the plurality of washed RNA-bound third solid supports and DNase to provide the plurality of DNase-treated second solid supports, the plurality of DNase-treated third solid supports and the flowthrough.

In some embodiments, the DNase is DNase I. In some embodiments, the DNase is RNase-free DNase. In some embodiments, the DNase I is RNase-free DNase I. The DNase, e.g., DNase I, is commercially available, e.g., from Norgen Biotek Corp.

In some embodiments, the performing the centrifugation of the washed RNA-bound second solid support and the DNase comprises subjecting the washed RNA-bound second solid support and the DNase to a centrifugal force of from about 500×g to about 5,000×g, from about 2,000×g to about 4,000×g, or from about 2,500×g to about 3,500×g. In some embodiments, the performing the centrifugation of the washed RNA-bound second solid support and the DNase comprises subjecting the washed RNA-bound second solid support and the DNase to a centrifugal force of about 1,000×g, about 2,000×g, about 3,000×g, 4,000×g, about 5,000×g, or more. In some embodiments, the performing the centrifugation of the washed RNA-bound second solid support and the DNase comprises subjecting the washed RNA-bound second solid support and the DNase to a centrifugal force of about 3,146×g. In some embodiments, the washed RNA-bound second solid support is one of a plurality of washed RNA-bound second solid supports, and the methods comprise performing a centrifugation of the plurality of washed RNA-bound second solid supports and the DNase at a centrifugal force of about 3,000×g or about 3,146×g.

In some embodiments, the performing the centrifugation of the washed RNA-bound second solid support, the washed RNA-bound third solid support and the DNase comprises subjecting the washed RNA-bound second solid support, the washed RNA-bound third solid support and the DNase to a centrifugal force of from about 500×g to about 5,000×g, from about 2,000×g to about 4,000×g, or from about 2,500×g to about 3,500×g. In some embodiments, the performing the centrifugation of the washed RNA-bound second solid support, the washed RNA-bound third solid support and the DNase comprises subjecting the washed RNA-bound second solid support, the washed RNA-bound third solid support and the DNase to a centrifugal force of about 1,000×g, about 2,000×g, about 3,000×g, 4,000×g, about 5,000×g, or more. In some embodiments, the performing the centrifugation of the washed RNA-bound second solid support, the washed RNA-bound third solid support and the DNase comprises subjecting the washed RNA-bound second solid support, the washed RNA-bound third solid support and the DNase to a centrifugal force of about 3,146×g. In some embodiments, the washed RNA-bound second solid support is one of a plurality of washed RNA-bound second solid supports, the washed RNA-bound third solid support is one of a plurality of washed RNA-bound third solid supports and the methods comprise performing a centrifugation of the plurality of washed RNA-bound second solid supports, the plurality of washed RNA-bound third solid supports and the DNase at a centrifugal force of about 3,000×g or about 3,146×g.

In some embodiments, the performing the centrifugation of the washed RNA-bound second solid support and the DNase occurs at a temperature of about 20° C. to about 30° C. In some embodiments, the performing the centrifugation of the washed RNA-bound second solid support and the DNase occurs at a temperature of about 20° C., about 21° C., about 22° C., about 23° C., about 24° C., about 25° C., about 26° C., about 27° C., about 28° C., about 29° C., or about 30° C. In some embodiments, the performing the centrifugation of the washed RNA-bound second solid support and the DNase occurs at a temperature of about 23° C. to about 30° C. In some embodiments, the performing the centrifugation of the washed RNA-bound second solid support and the DNase occurs at a temperature of about 23° C. In some embodiments, the performing the centrifugation of the washed RNA-bound second solid support and the DNase occurs at a temperature of about 30° C. In some embodiments, the washed RNA-bound second solid support is one of a plurality of washed RNA-bound second solid supports, and the methods comprise performing a centrifugation of the plurality of washed RNA-bound second solid supports and the DNase at a temperature of about 23° C. to about 30° C. In some embodiments, the methods comprise performing a centrifugation of the plurality of washed RNA-bound second solid supports and the DNase at a temperature of about 23° C. In some embodiments, the methods comprise performing a centrifugation of the plurality of washed RNA-bound second solid supports and the DNase at a temperature of about 30° C.

In some embodiments, the performing the centrifugation of the washed RNA-bound second solid support, the washed RNA-bound third solid support and the DNase occurs at a temperature of about 20° C. to about 30° C. In some embodiments, the performing the centrifugation of the washed RNA-bound second solid support, the washed RNA-bound third solid support and the DNase occurs at a temperature of about 20° C., about 21° C., about 22° C., about 23° C., about 24° C., about 25° C., about 26° C., about 27° C., about 28° C., about 29° C., or about 30° C. In some embodiments, the performing the centrifugation of the washed RNA-bound second solid support, the washed RNA-bound third solid support and the DNase occurs at a temperature of about 23° C. to about 30° C. In some embodiments, the performing the centrifugation of the washed RNA-bound second solid support, the washed RNA-bound third solid support and the DNase occurs at a temperature of about 23° C. In some embodiments, the performing the centrifugation of the washed RNA-bound second solid support, the washed RNA-bound third solid support and the DNase occurs at a temperature of about 30° C. In some embodiments, the washed RNA-bound second solid support is one of a plurality of washed RNA-bound second solid supports, the washed RNA-bound third solid support is one of a plurality of washed RNA-bound third solid supports, and the methods comprise performing a centrifugation of the plurality of washed RNA-bound second solid supports, the plurality of washed RNA-bound third solid supports and the DNase at a temperature of about 23° C. to about 30° C. In some embodiments, the methods comprise performing a centrifugation of the plurality of washed RNA-bound second solid supports, the plurality of washed RNA-bound third solid supports and the DNase at a temperature of about 23° C. In some embodiments, the methods comprise performing a centrifugation of the plurality of washed RNA-bound second solid supports, washed RNA-bound third solid supports and the DNase at a temperature of about 30° C.

In some embodiments, the performing the centrifugation of the washed RNA-bound second solid support and the DNase is for about 1 minute, about 2 minutes, about 3 minutes, about 4 minutes, about 5 minutes, about 6 minutes, about 7 minutes, about 8 minutes, about 9 minutes, about 10 minutes, or more. In some embodiments, the performing the centrifugation of the washed RNA-bound second solid support and the DNase is for about 2 minutes.

In some embodiments, the performing the centrifugation of the washed RNA-bound second solid support, RNA-bound third solid support and the DNase is for about 1 minute, about 2 minutes, about 3 minutes, about 4 minutes, about 5 minutes, about 6 minutes, about 7 minutes, about 8 minutes, about 9 minutes, about 10 minutes, or more. In some embodiments, the performing the centrifugation of the washed RNA-bound second solid support, washed RNA-bound third solid support and the DNase is for about 2 minutes.

In some embodiments, the flowthrough is a first flowthrough. In some embodiments, the treating further comprises admixing the DNase-treated second solid support and the first flowthrough to provide a DNase-treated second solid support admixture, and incubating the DNase-treated second solid support admixture to provide an incubated DNase-treated solid support admixture. In some embodiments, the treating further comprises admixing the DNase-treated second solid support, the DNase-treated third solid support and the first flowthrough to provide a DNase-treated second solid support and DNase-treated third solid support admixture, and incubating the DNase-treated second solid support and DNase-treated third solid support admixture to provide an incubated DNase-treated solid support admixture.

In some embodiments, the incubating occurs at room temperature. In some embodiments, the incubating occurs at a temperature of about 18° C., about 19° C., about 20° C., about 21° C., about 22° C., about 23° C., about 24° C., about 25° C., about 26° C., about 27° C., about 28° C., about 29° C., or about 30° C. t. In some embodiments, the incubating occurs for about 1 minute, about 5 minutes, about 10 minutes, about 15 minutes, about 20 minutes, about 25 minutes, or about 30 minutes. In some embodiments, the incubating occurs for about 15 minutes.

In some embodiments, the methods further comprise admixing the incubated DNase-treated solid support admixture and a wash buffer to provide a DNase-treated second solid support and wash buffer admixture, and performing a centrifugation of the DNase-treated second solid support and wash buffer admixture to provide the washed DNase-treated second solid support and a second flowthrough.

In some embodiments, the DNase-treated second solid support is one of a plurality of DNase-treated second solid supports, and the methods comprise washing the plurality of DNase-treated second solid supports to provide a plurality of washed DNase-treated second solid supports. In some embodiments, the DNase-treated second solid support is one of a plurality of DNase-treated second solid supports, the DNase-treated third solid support is one of a plurality of DNase-treated third solid supports, and the methods comprise washing the plurality of DNase-treated second solid supports and the plurality of DNase-treated third solid supports to provide a plurality of washed DNase-treated second solid supports and a plurality of washed DNase-treated third solid supports.

In some embodiments, the methods further comprise admixing the incubated DNase-treated support admixture and a wash buffer to provide a DNase-treated second solid support, DNase-treated third solid support and wash buffer admixture, and performing a centrifugation of the DNase-treated second solid support, DNase-treated third solid support and wash buffer admixture to provide the washed DNase-treated second solid support, the washed DNase-treated third solid support and a second flowthrough.

In some embodiments, the methods further comprise removing and optionally discarding one or more of the flowthrough, first flowthrough and second flowthrough after performing the centrifugation. In some embodiments, one or more of the flowthrough, first flowthrough and second flowthrough are removed by pipetting. In some embodiments, one or more of the flowthrough, first flowthrough and second flowthrough are removed by decanting. In some embodiments, the wash buffer has a volume of about 50 μL, about 100 μL, about 150 μL, about 200 μL, about 250 μL, about 300 μL, about 350 μL, about 400 μL, about 450 μL, about 500 μL, about 550 μL, about 600 μL, about 650 μL, about 700 μL, about 750 μL, about 800 μL, about 850 μL, about 900 μL, about 950 μL, about 1000 μL, or more. In some embodiments, the wash buffer has a volume of about 500 μL.

In some embodiments, the performing the centrifugation of the DNase-treated second solid support and wash buffer admixture comprises subjecting the DNase-treated second solid support and wash buffer admixture to a centrifugal force of from about 500×g to about 5,000×g, from about 2,000× g to about 4,000×g, or about 2,500×g to about 3,500×g. In some embodiments, the performing the centrifugation of the DNase-treated second solid support and wash buffer admixture comprises subjecting the DNase-treated second solid support and wash buffer admixture to a centrifugal force of about 1,000×g, about 2,000×g, about 3,000×g, 4,000×g, about 5,000×g, or more. In some embodiments, the performing the centrifugation of the DNase-treated second solid support and wash buffer admixture comprises subjecting the DNase-treated second solid support and wash buffer admixture to a centrifugal force of about 3,146×g.

In some embodiments, the DNase-treated second solid support is one of a plurality of DNase-treated second solid supports, and the methods comprise performing a centrifugation of the plurality of DNase-treated second solid supports and wash buffer admixture at a centrifugal force of about 3,000×g or about 3,146×g.

In some embodiments, the performing the centrifugation of the DNase-treated second solid support, DNase-treated third solid support and wash buffer admixture comprises subjecting the DNase-treated second solid support, DNase-treated third solid support and wash buffer admixture to a centrifugal force of from about 500×g to about 5,000×g, from about 2,000×g to about 4,000×g, or about 2,500×g to about 3,500×g. In some embodiments, the performing the centrifugation of the DNase-treated second solid support, DNase-treated third solid support and wash buffer admixture comprises subjecting the DNase-treated second solid support, DNase-treated third solid support and wash buffer admixture to a centrifugal force of about 1,000×g, about 2,000×g, about 3,000×g, 4,000×g, about 5,000×g, or more. In some embodiments, the performing the centrifugation of the DNase-treated second solid support, DNase-treated third solid support and wash buffer admixture comprises subjecting the DNase-treated second solid support, DNase-treated third solid support and wash buffer admixture to a centrifugal force of about 3,146×g.

In some embodiments, the DNase-treated second solid support is one of a plurality of DNase-treated second solid supports, the DNase-treated third solid support is one of a plurality of DNase-treated third solid supports, and the methods comprise performing a centrifugation of the plurality of DNase-treated second solid supports, the plurality of DNase-treated third solid supports and wash buffer admixture at a centrifugal force of about 3,000×g or about 3,146×g.

In some embodiments, the performing the centrifugation of the DNase-treated second solid support and wash buffer admixture occurs at a temperature of about 20° C. to about 30° C. In some embodiments, the performing the centrifugation of the DNase-treated second solid support and wash buffer admixture occurs at a temperature of about 20° C., about 21° C., about 22° C., about 23° C., about 24 ° C., about 25° C., about 26° C., about 27° C., about 28° C., about 29° C., or about 30° C. In some embodiments, the performing the centrifugation of the DNase-treated second solid support and wash buffer admixture occurs at a temperature of about 23° C. to about 30° C. In some embodiments, the performing the centrifugation of the DNase-treated second solid support and wash buffer admixture occurs at a temperature of about 23° C. In some embodiments, the performing a centrifugation of the DNase-treated second solid support and wash buffer admixture occurs at a temperature of about 30° C.

In some embodiments, the performing the centrifugation of the DNase-treated second solid support, DNase-treated third solid support and wash buffer admixture occurs at a temperature of about 20° C. to about 30° C. In some embodiments, the performing the centrifugation of the DNase-treated second solid support, DNase-treated third solid support and wash buffer admixture occurs at a temperature of about 20° C., about 21° C., about 22° C., about 23° C., about 24° C., about 25° C., about 26° C., about 27° C., about 28° C., about 29° C., or about 30° C. In some embodiments, the performing the centrifugation of the DNase-treated second solid support, DNase-treated third solid support and wash buffer admixture occurs at a temperature of about 23° C. to about 30° C. In some embodiments, the performing the centrifugation of the DNase-treated second solid support, DNase-treated third solid support and wash buffer admixture occurs at a temperature of about 23° C. In some embodiments, the performing a centrifugation of the DNase-treated second solid support, DNase-treated third solid support and wash buffer admixture occurs at a temperature of about 30° C.

In some embodiments, the DNase-treated second solid support is one of a plurality of DNase-treated second solid supports, and the methods comprise performing a centrifugation of the plurality of DNase-treated second solid supports and wash buffer admixture at a temperature of about 23° C. to about 30° C. In some embodiments, the methods comprise performing a centrifugation of the plurality of DNase-treated second solid supports and wash buffer admixture at a temperature of about 23° C. In some embodiments, the methods comprise performing a centrifugation of the plurality of DNase-treated second solid supports and wash buffer admixture at a temperature of about 30° C.

In some embodiments, the DNase-treated second solid support is one of a plurality of DNase-treated second solid supports, the DNase-treated third solid support is one of a plurality of DNase-treated third solid supports, and the methods comprise performing a centrifugation of the plurality of DNase-treated second solid supports, plurality of DNase-treated third solid supports and wash buffer admixture at a temperature of about 23° C. to about 30° C. In some embodiments, the methods comprise performing a centrifugation of the plurality of DNase-treated second solid supports, plurality of DNase-treated third solid supports and wash buffer admixture at a temperature of about 23° C. In some embodiments, the methods comprise performing a centrifugation of the plurality of DNase-treated second solid supports, plurality of DNase-treated third solid supports and wash buffer admixture at a temperature of about 30° C.

In some embodiments, the performing the centrifugation is for about 1 minute, about 2 minutes, about 3 minutes, about 4 minutes, about 5 minutes, about 6 minutes, about 7 minutes, about 8 minutes, about 9 minutes, about 10 minutes, or more. In some embodiments, the performing a centrifugation of the DNase-treated second solid support and the wash buffer admixture is for about 2 minutes.

In some embodiments, the methods further comprise repeating the steps of admixing the DNase-treated second solid support and the wash buffer and performing a centrifugation to provide the washed DNase-treated second solid support. In some embodiments, the methods comprise repeating once the steps of admixing the DNase-treated second solid support and the wash buffer and performing a centrifugation to provide the washed DNase-treated second solid support, and, accordingly, comprise these two admixing steps. In some embodiments, the methods comprise repeating only once the steps of admixing the DNase-treated second solid support and the wash buffer and performing a centrifugation to provide the washed DNase-treated second solid support, and, accordingly, comprise no more than these two admixing steps. In some embodiments, the methods comprise repeating twice the steps of admixing the DNase-treated second solid support and the wash buffer and performing a centrifugation to provide the washed DNase-treated second solid support, and accordingly, comprise these three admixing steps. In some embodiments, the methods comprise repeating only twice the steps of admixing the DNase-treated second solid support and the wash buffer and performing a centrifugation to provide the washed DNase-treated second solid support, and, accordingly, comprise no more than these three admixing steps.

In some embodiments, the methods further comprise repeating the steps of admixing the plurality of DNase-treated second solid supports and the wash buffer and performing a centrifugation to provide a plurality of washed DNase-treated second solid supports. In some embodiments, the methods comprise repeating once the steps of admixing the plurality of DNase-treated second solid supports and the wash buffer and performing a centrifugation to provide the plurality of washed DNase-treated second solid supports, and, accordingly, comprise these two admixing steps. In some embodiments, the methods comprise repeating only once the steps of admixing the plurality of DNase-treated second solid supports and the wash buffer and performing a centrifugation to provide the plurality of washed DNase-treated second solid supports, and, accordingly, comprise no more than these two admixing steps. In some embodiments, the methods comprise repeating twice the steps of admixing the plurality of DNase-treated second solid supports and the wash buffer and performing a centrifugation to provide the plurality of washed DNase-treated second solid supports, and accordingly, comprise these three admixing steps. In some embodiments, the methods comprise repeating only twice the steps of admixing the plurality of DNase-treated second solid supports and the wash buffer and performing a centrifugation to provide the plurality of washed DNase-treated second solid supports, and, accordingly, comprise no more than these three admixing steps.

In some embodiments, the methods further comprise repeating the steps of admixing the DNase-treated second solid support, the DNase-treated third solid support and the wash buffer, and performing a centrifugation to provide the washed DNase-treated second solid support and the washed DNase-treated third solid support. In some embodiments, the methods comprise repeating once the steps of admixing the DNase-treated second solid support, the DNase-treated third solid support and the wash buffer and performing a centrifugation to provide the washed DNase-treated second solid support and the washed DNase-treated third solid support, and, accordingly, comprise these two admixing steps. In some embodiments, the methods comprise repeating only once the steps of admixing the DNase-treated second solid support, the DNase-treated third solid support and the wash buffer and performing a centrifugation to provide the washed DNase-treated second solid support and the washed DNase-treated third solid support, and, accordingly, comprise no more than these two admixing steps. In some embodiments, the methods comprise repeating twice the steps of admixing the DNase-treated second solid support, the DNase-treated third solid support and the wash buffer and performing a centrifugation to provide the washed DNase-treated second solid support and the washed DNase-treated third solid, and, accordingly, comprise these three admixing steps. In some embodiments, the methods comprise repeating only twice the steps of admixing the DNase-treated second solid support, the DNase-treated third solid support and the wash buffer and performing a centrifugation to provide the washed DNase-treated second solid support and the washed DNase-treated third solid, and, accordingly, comprise no more than these three admixing steps.

In some embodiments, the methods further comprise repeating the steps of admixing the plurality of DNase-treated second solid supports, the plurality of DNase-treated third solid supports and the wash buffer, and performing a centrifugation to provide the plurality of washed DNase-treated second solid supports and the plurality of washed DNase-treated third solid supports. In some embodiments, the methods comprise repeating once the steps of admixing the plurality of DNase-treated second solid supports, the plurality of DNase-treated third solid supports and the wash buffer and performing a centrifugation to provide the plurality of washed DNase-treated second solid supports and the plurality of washed DNase-treated third solid supports, and, accordingly, comprise these two admixing steps. In some embodiments, the methods comprise repeating only once the steps of admixing the plurality of DNase-treated second solid supports, the plurality of DNase-treated third solid supports and the wash buffer and performing a centrifugation to provide the plurality of washed DNase-treated second solid supports and the plurality of washed DNase-treated third solid supports, and, accordingly, comprise no more than these two admixing steps. In some embodiments, the methods comprise repeating twice the steps of admixing the plurality of DNase-treated second solid supports, the plurality of DNase-treated third solid supports and the wash buffer and performing a centrifugation to provide the plurality of washed DNase-treated second solid supports and the plurality of washed DNase-treated third solids, and, accordingly, comprise these three admixing steps. In some embodiments, the methods comprise repeating only twice the steps of admixing the plurality of DNase-treated second solid supports, the plurality of DNase-treated third solid supports and the wash buffer and performing a centrifugation to provide the plurality of washed DNase-treated second solid supports and the plurality of washed DNase-treated third solid supports, and, accordingly, comprise no more than these three admixing steps.

In some embodiments, the methods comprise drying the washed DNase-treated second solid support to provide a dried second solid support. In some embodiments, the drying comprises performing a centrifugation of the washed DNase-treated solid support to provide a dried second solid support. In some embodiments, the dried second solid support comprises, e.g., on its surface, no detectable amount of remaining polar organic solvent, e.g., ethanol. In some embodiments, the dried second solid support is one of a plurality of dried second solid supports. In some embodiments, the methods comprise drying a plurality of washed DNase-treated solid supports to provide a plurality of dried second solid supports.

In some embodiments, the methods comprise drying the washed DNase-treated second solid support and the washed DNase-treated third solid support to provide a dried second solid support and a dried third solid support. In some embodiments, the drying comprises performing a centrifugation of the washed DNase-treated second solid support and the washed DNase-treated third solid support to provide the dried second solid support and the dried third solid support. In some embodiments, either the dried second solid support or the dried third solid support comprises, e.g., on its surface, a detectable amount of remaining polar organic solvent, e.g., ethanol. In some embodiments, neither the dried second solid support nor the dried third solid support comprises, e.g., on its surface, any detectable amount of remaining polar organic solvent, e.g., ethanol. In some embodiments, the dried second solid support is one of a plurality of dried second solid supports, and the dried third solid support is one of a plurality of dried third solid supports. In some embodiments, the methods comprise drying a plurality of washed DNase-treated second solid supports and a plurality of washed DNase-treated third solid supports to provide a plurality of dried second solid supports and a plurality of dried third solid supports.

In some embodiments, the drying comprises performing a centrifugation of the washed DNase-treated second solid support, and optionally the washed DNase-treated third solid support, at a centrifugal force of from about 500×g to about 5,000×g, from about 2,000×g to about 4,000×g, or about 2,500×g to about 3,500×g. In some embodiments, the drying comprises performing the centrifugation at a centrifugal force of about 1,000×g, about 2,000×g, about 3,000×g, 4,000×g, about 5,000×g, or more. In some embodiments, the drying comprises performing the centrifugation at a centrifugal force of about 3,146×g.

In some embodiments, the washed DNase-treated second solid support is one of a plurality of washed DNase-treated second solid supports, and the methods comprise performing a centrifugation of the plurality of washed DNase-treated second solid supports at a centrifugal force of about 3,000×g (e.g., 3,146×g). In some embodiments, the washed DNase-treated second solid support is one of a plurality of washed DNase-treated second solid supports, the washed DNase-treated third solid support is one of a plurality of washed DNase-treated third solid supports, and the methods comprise performing a centrifugation of the plurality of washed DNase-treated second solid supports and the plurality of DNase-treated third solid supports at a centrifugal force of about 3,000×g (e.g., 3,146×g).

In some embodiments, the drying comprises performing the centrifugation for up to about 30 minutes. In some embodiments, the drying comprises performing the centrifugation for up to about 20 minutes. In some embodiments, the drying comprises performing the centrifugation for about 5 minutes to about 30 minutes. In some embodiments, the drying comprises performing the centrifugation for about 10 minutes to about 20 minutes. In some embodiments, the drying comprises performing the centrifugation for about 5 minutes to about 15 minutes. In some embodiments, the drying comprises performing the centrifugation for about 5 minutes, about 6 minutes, about 7 minutes, about 8 minutes, about 9 minutes, about 10 minutes, 11 minutes, about 12 minutes, about 13 minutes, about 14 minutes, about 15 minutes, about 16 minutes, about 17 minutes, about 18 minutes, 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, or more. In some embodiments, the drying comprises performing the centrifugation for about 15 minutes.

In some embodiments, the drying comprises performing the centrifugation at a temperature of about 20° C. to about 30° C. In some embodiments, the drying comprises performing the centrifugation at a temperature of about 20° C., about 21° C., about 22° C., about 23° C., about 24° C., about 25° C., about 26 ° C., about 27° C., about 28° C., about 29° C., or about 30° C. In some embodiments, the drying comprises performing the centrifugation at a temperature of about 23° C. to about 30° C. In some embodiments, the drying comprises performing the centrifugation at a temperature of about 23° C. In some embodiments, the drying comprises performing the centrifugation at a temperature of about 30° C.

In some embodiments, the methods comprise eluting RNA from the dried second solid support to provide extracted RNA. In some embodiments, the methods comprise eluting RNA from the dried second solid support and the dried third solid support to provide extracted RNA. In some embodiments, the eluting comprises admixing the dried second solid support and an elution buffer to provide an elution support admixture, incubating the elution support admixture to provide an incubated elution support admixture, and performing a centrifugation of the incubated elution support admixture to provide the extracted RNA. In some embodiments, the eluting comprises admixing the dried second solid support, the dried third solid support and an elution buffer to provide an elution support admixture, incubating the elution support admixture to provide an incubated elution support admixture, and performing a centrifugation of the incubated elution support admixture to provide the extracted RNA. In some embodiments, the admixing comprises allowing the elution buffer to contact the dried second solid support and optionally the dried third solid support. In some embodiments, the admixing comprises pipetting the elution buffer onto the dried second solid support and optionally onto the dried third solid support. In some embodiments, the method further comprises eluting the RNA, e.g., at least once, at least twice, at least three times, or more than three times.

In some embodiments, the dried second solid support is one of a plurality of dried second solid supports, and the eluting comprises admixing the plurality of dried second solid supports and an elution buffer to provide the elution support admixture, incubating the elution support admixture to provide the incubated elution support admixture, and performing a centrifugation of the incubated elution support admixture to provide the extracted RNA. In some embodiments, the methods comprise eluting RNA from a plurality of dried second solid supports to provide extracted RNA.

In some embodiments, the dried second solid support is one of a plurality of dried second solid supports, the dried third solid support is one of a plurality of dried third solid supports, and the eluting comprises admixing the plurality of dried second solid supports, the plurality of dried third solid supports and an elution buffer to provide the elution support admixture, incubating the elution support admixture to provide the incubated elution support admixture, and performing a centrifugation of the incubated elution support admixture to provide the extracted RNA. In some embodiments, the methods comprise eluting RNA from a plurality of dried second solid supports and a plurality of dried third solid supports to provide the extracted RNA, in some embodiments, in an elution buffer and extracted RNA admixture. In some embodiments, the extracted RNA is suspended in the elution buffer and extracted RNA admixture. In some embodiments, the extracted RNA is dissolved in the elution buffer and extracted RNA admixture.

In some embodiments, the elution buffer is an aqueous elution buffer. In some embodiments, the elution buffer is Tris-EDTA (TE) buffer. In some embodiments, the TE buffer comprises about 10 mM Tris-HCl and about 0.1 mM EDTA. In some embodiments, the TE buffer is about 10 mM Tris-HCl and about 0.1 mM EDTA. In some embodiments, the elution buffer is Tris-HCl buffer. In some embodiments, the Tris-HCl buffer comprises about 10 mM Tris-HCl. In some embodiments, the elution buffer is Tris-Cl buffer. In some embodiments, the elution buffer is water. In some embodiments, the elution buffer is RNase-free water. Elution buffers are commercially available, e.g., from Thermo Fisher Scientific or Norgen.

In some embodiments, the elution buffer has a volume of about 25 μL, 50 μL, 75 μL, about 100 μL, about 150 μL, about 200 μL, about 250 μL, about 300 μL, about 350 μL, about 400 μL, about 450 μL, or about 500 μL. In some embodiments, the elution buffer has a volume of about 100 μL.

In some embodiments, the elution buffer has a temperature of from about 4° C. to about 70° C. In some embodiments, the elution buffer has a temperature of about 5° C., about 10° C., about 15° C., about 20° C., about 25° C., about 30° C., about 35° C., about 40° C., about 45° C., about 50° C., about 55° C., about 60° C., or about 70° C. In some embodiments, the elution buffer has a temperature of from about 15° C. to about 37° C. In some embodiments, the elution buffer has a temperature of from about 20° C. to about 37° C. In some embodiments, the elution buffer has a temperature of about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or about 37° C. In some embodiments, the elution buffer has a temperature of about 23° C. In some embodiments, the elution buffer has a temperature of about 30° C. In some embodiments, the elution buffer is at room temperature.

In some embodiments, the elution buffer has a pH of from about 6.5 to about 8.5. In some embodiments, the elution buffer has a pH of from about 7 to about 8.5. In some embodiments, the elution buffer has a pH of from about 7 to about 8. In some embodiments, the elution buffer has a pH of from about 7 to about 8. In some embodiments, the elution buffer has a pH of about 6.5, about 6.6, about 6.7, about 6.8, about 6.9, about 7.0, about 7.1, about 7.2 about 7.3, about 7.4, about 7.5, about 7.6, about 7.7, about 7.8, about 7.9, about 8.0, about 8.1, about 8.2 about 8.3, about 8.4, or about 8.5. In some embodiments, the elution buffer has a pH of about 7. In some embodiments, the elution buffer is water.

In some embodiments, incubating the elution support admixture is for about 30 seconds, about 1 minute, about 2 minutes, about 3 minutes, about 4 minutes, about 5 minutes, about 6 minutes, about 7 minutes, about 8 minutes, about 9 minutes, about 10 minutes, or more. In some embodiments, incubating the elution support admixture is for about 2 minutes.

In some embodiments, the performing the centrifugation of the incubated elution support admixture is at a centrifugal force of about 100 rpm to about 2000 rpm. In some embodiments, the performing the centrifugation of the incubated elution support admixture is at a centrifugal force of about 250 rpm to about 750 rpm. In some embodiments, the performing the centrifugation of the incubated elution support admixture is at a centrifugal force of about 100 rpm, about 200 rpm, about 300 rpm, about 400 rpm, about 500 rpm, about 600 rpm, about 700 rpm, about 800 rpm, about 900 rpm, or about 1000 rpm. In some embodiments, the performing the centrifugation of the incubated elution support admixture is at a centrifugal force of about 500 rpm.

In some embodiments, the performing the centrifugation of the incubated elution support admixture is for about 1 minute, about 2 minutes, about 3 minutes, about 4 minutes, about 5 minutes, about 6 minutes, about 7 minutes, about 8 minutes, about 9 minutes, about 10 minutes, or more. In some embodiments, the performing the centrifugation of the incubated elution support admixture is for about 2 minutes.

In some embodiments, the performing the centrifugation of the incubated elution support admixture occurs at a temperature of about 20° C. to about 30° C. In some embodiments, the performing the centrifugation of the incubated elution support admixture occurs at a temperature of about 20° C., about 21° C., about 22° C., about 23° C., about 24° C., about 25° C., about 26° C., about 27° C., about 28° C., about 29° C., or about 30° C. In some embodiments, the performing the centrifugation of the incubated elution support admixture occurs at a temperature of about 23° C. to about 30° C. In some embodiments, the performing the centrifugation of the incubated elution support admixture occurs at a temperature of about 23° C. In some embodiments, the performing the centrifugation of the incubated elution support admixture occurs at a temperature of about 30° C.

In some embodiments, the performing the centrifugation of the incubated elution support admixture is a first centrifugation of the incubated elution support admixture, and the method further comprises performing a second centrifugation of the incubated elution support admixture. In some embodiments, the first centrifugation is at a low speed (e.g., about 500 rpm). In some embodiments, the second centrifugation is at a high centrifugal force (e.g., about 3,000×g).

In some embodiments, the performing the second centrifugation of the incubated elution support admixture is at a centrifugal force of from about 500×g to about 5,000×g, from about 2,000×g to about 4,000×g, or from about 2,500×g to about 3,500×g. In some embodiments, the performing the second centrifugation of the incubated elution support admixture is at a centrifugal force of about 1,000×g, about 2,000×g, about 3,000×g, 4,000×g, about 5,000×g, or more. In some embodiments, the performing the second centrifugation of the incubated elution support admixture is at a centrifugal force of about 3,146×g.

In some embodiments, the performing the second centrifugation of the incubated elution support admixture is for about 1 minute, about 2 minutes, about 3 minutes, about 4 minutes, about 5 minutes, about 6 minutes, about 7 minutes, about 8 minutes, about 9 minutes, about 10 minutes, or more. In some embodiments, the performing the second centrifugation of the incubated elution support admixture is for about 2 minutes.

In some embodiments, the performing the second centrifugation of the incubated elution support admixture occurs at a temperature of about 20° C. to about 30° C. In some embodiments, the performing the second centrifugation of the incubated elution support admixture occurs at a temperature of about 20° C., about 21° C., about 22° C., about 23° C., about 24° C., about 25° C., about 26° C., about 27° C., about 28° C., about 29° C., or about 30° C. In some embodiments, the performing the second centrifugation of the incubated elution support admixture occurs at a temperature of about 23° C. to about 30° C. In some embodiments, the performing the second centrifugation of the incubated elution support admixture occurs at a temperature of about 23° C. In some embodiments, the performing the second centrifugation of the incubated elution support admixture occurs at a temperature of about 30° C.

Without wishing to be bound by theory, it is believed that the washing steps useful in the methods described herein can remove one or more contaminates, e.g., primarily alcohol, guanidinium chloride or guanidinium thiocyanate.

In some embodiments, the wash buffer comprises a salt. In some embodiments, the wash buffer comprises the salt at a concentration of from about 0.01 to about 2.5 M. In some embodiments, the salt is NaCl. In some embodiments, the wash buffer comprises a salt, e.g., NaCl, and tris(hydroxymethyl)aminomethane (Tris).

Suitable wash buffers can include a variety of one or more components. In some embodiments, the wash buffer comprises one or more of the following reagents: Tris, Bis-Tris, Bis-Tris-Propane, imidazole, citrate, methylmalonic acid, acetic acid, ethanol, ethanolamine, diethanolamine, triethanolamine (TEA) and sodium phosphate. In some embodiments, the wash buffer comprises ethanol.

Additional components that may be present in wash buffers include non-ionic surfactants or detergents, ionic or zwitter-ionic surfactants or detergents, chaotropic salts (e.g., guanidinium thiocyanate or guanidinium chloride), disulfide bond reducing agents, proteases, nucleases, and other additives or components that digest, denature, disrupt, or degrade for the purpose of extracting, purifying, enriching, or otherwise isolating nucleic acids. Non-ionic surfactants or detergents can include, but are not limited to, surfactants from the following classes: octylphenol ethoxylate, polysorbate, poloxamer, or polyoxyethylene.

Octylphenol ethoxylate surfactants can include, but are not limited to, branched octylphenoxy polyethoxy ethanol (e.g., IGEPAL CA-630), t-octylphenoxypolyethoxyethanol (e.g., Triton X-100), or other polyethylene oxide chains with an aromatic hydrocarbon lipophilic or hydrophobic group. Polysorbate surfactants can include but are not limited to polyethylene glycol sorbitan monolaurate (e.g., Tween 20), polyethylene glycol sorbitan monooleate (e.g., Tween 80), or sorbitan monooleate (e.g., Span 80). Poloxamer surfactants can include, but are not limited to, polyoxyethylene-polyoxypropylene block copolymer (e.g., Pluronic F-68) or polyethylene-polypropylene glycol block copolymer (e.g., Pluronic F-127). Polyoxyethylene surfactants can include, but are not limited to, nonylphenoxy-polyethoxylethanol (e.g., NP-40).

Non-ionic surfactants or detergents can include but are not limited to IGEPAL (e.g., IGEPAL CA-630), Triton X-100, Tween 20, Tween 80, NP-40, other block copolymers including Pluronic (e.g., F-68 or F-127), Span 80, and pegylated polymers or copolymers. Non-ionic surfactants or detergents can be used to reduce or prevent biological molecule adsorption to channel walls, or to control wetting and/or surface tension properties of fluids to control loading of sample into fluidic devices. Non-ionic surfactants or detergents can be present at concentrations from about 0.0005-5% v/v or w/v. Ionic surfactants or detergents can include but are not limited to sodium dodecyl sulfate (e.g., at 0.01-2% w/v), sodium dodecylbenzenesulfonate (e.g., at 0.01-2% w/v), sodium cholesteryl sulfate (e.g., at 0.01%-2% w/v), and sodium deoxycholate (e.g., at about 10-1000 mM). Chaotropic agents can include but are not limited to urea (e.g., at about 0.5-9.5 M, or in some cases, 5-9.5 M) thiourea, butanol, ethanol, guanidine, guanidinium chloride, guanidinium thiocyanate, lithium perchlorate, lithium acetate, lithium chloride, magnesium chloride, phenol, and propanol. Disulfide bond reducing agents can include, but are not limited to, DTT (e.g., at about 0.1-40 mM, or about 10 mM) and beta-mercaptoethanol (e.g., at about 0 to about 5%). Proteases can include but are not limited to Proteinase K, proteases, endoproteinases (e.g., trypsin, LysC, GluC, AspN), peptidases, pepsin, and papain. Nucleases can include but are not limited to non-specific nucleic acid digestion enzymes such as DNases including DNase I (e.g., to prepare DNA-free RNA extractions) and RNase, such as RNase A, RNase T, or combinations thereof (e.g., to prepare RNA-free DNA extractions). Nucleases can also include specific nucleic acid digestion enzymes (e.g., restriction enzymes) which can cut at specific nucleic acid sequences and can produce predictable fragment sizes and fragment size distributions.

In some embodiments, where the extracted RNA is present in an elution buffer and extracted RNA admixture, the extracted RNA concentration can is increased, for example, comprising precipitating the extracted RNA to provided precipitated RNA and subsequently reconstituting the precipitated RNA using a relatively smaller volume of buffer. In some embodiments, the extracted RNA concentration can be increased, for example, by using a vacuum concentrator.

In some embodiments, the extracted RNA is not separated by size prior to its detection. In some embodiments, the extracted RNA is not concentrated prior to its detection.

In some embodiments, the extracted RNA is provided in an amount that is at least 1.5-fold greater, 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, or more, than extracted RNA obtained from a method that does not comprise admixing whole urine and a solid support comprising silicon carbide.

Also provided are compositions comprising whole urine and a solid support comprising silicon carbide. In some embodiments, the whole urine is from a urine sample of a male human subject who has a prostate and who did not have a digital rectal examination (DRE) within about 180 minutes before providing the urine sample. In some embodiments, the solid support is one of a plurality of solid supports, and the composition comprises whole urine and the plurality of solid supports. In some embodiments, the plurality of solid supports is a plurality of silicon carbide particles. In some embodiments, the compositions further comprise a preservative. In some embodiments, the preservative is UCM. In some embodiments, the preservative is UAS. In some embodiments, the compositions further comprise a lysis buffer. In some embodiments, the lysis buffer comprises guanidinium thiocyanate, guanidinium chloride or β-mercaptoethanol. In some embodiments, the lysis buffer comprises guanidinium thiocyanate and β-mercaptoethanol.

Further provided are compositions comprising urine and an RNA-bound solid support. In some embodiments, the RNA-bound support is an mRNA-bound support. In some embodiments, the RNA-bound support is one of a plurality of RNA-bound solid supports, and the composition comprises the plurality of RNA-bound supports. In some embodiments, the compositions further comprise a preservative. In some embodiments, the preservative is UCM. In some embodiments, the preservative is UAS. In some embodiments, the compositions further comprise a lysis buffer. In some embodiments, the lysis buffer comprises guanidinium thiocyanate, guanidinium chloride or β-mercaptoethanol. In some embodiments, the lysis buffer comprises guanidinium thiocyanate and β-mercaptoethanol.

In some embodiments, the compositions are made by a method comprising admixing whole urine and a solid support comprising silicon carbide to provide the RNA-bound solid support, wherein the admixing is not performed under reduced pressure. In some embodiments, the compositions are made by a method comprising admixing whole urine and a plurality of solid supports comprising silicon carbide to provide the plurality of RNA-bound solid supports, wherein the admixing is not performed under reduced pressure. In some embodiments, the reduced pressure is about 0.8 bar. In some embodiments, the method comprises admixing whole urine, a preservative, a lysis buffer, and a plurality of solid supports comprising silicon carbide, wherein the admixing is not performed under reduced pressure. In some embodiments, the method comprises admixing whole urine, a preservative, a lysis buffer, and silicon carbide particles, wherein the admixing is not performed under reduced pressure. In some embodiments, the lysis buffer comprises guanidinium thiocyanate, guanidinium chloride or (3-mercaptoethanol. In some embodiments, the lysis buffer comprises guanidinium thiocyanate and (3-mercaptoethanol. In some embodiments, the whole urine is from a urine sample of a male human subject who has a prostate and who did not have a digital rectal examination (DRE) within about 180 minutes before providing the urine sample.

In some embodiments, the present invention provides methods for making an RNA-bound solid support, comprising admixing whole urine and a solid support comprising silicon carbide to provide the RNA-bound solid support. In some embodiments, the methods comprise admixing whole urine, a preservative, a lysis buffer, and a solid support comprising silicon carbide. In some embodiments, the solid support is a silicon carbide particle. In some embodiments, the whole urine is from a urine sample of a male human subject who has a prostate and who did not have a digital rectal examination (DRE) within about 180 minutes before providing the urine sample. In some embodiments, the method does not comprise admixing the whole urine and the solid support under reduced pressure. In some embodiments, the reduced pressure is about 0.8 bar. In some embodiments, the lysis buffer comprises guanidinium thiocyanate, guanidinium chloride or β-mercaptoethanol. In some embodiments, the lysis buffer comprises guanidinium thiocyanate and β-mercaptoethanol.

In some embodiments, the present invention provides methods for making a plurality of RNA-bound solid supports, comprising admixing whole urine and a plurality of solid supports comprising silicon carbide to provide the plurality of RNA-bound solid supports. In some embodiments, the methods comprise admixing whole urine, a preservative, a lysis buffer, and a plurality of solid supports comprising silicon carbide. In some embodiments, the methods comprise admixing whole urine, a preservative, a lysis buffer, and a plurality of solid supports comprising silicon carbide. In some embodiments, the plurality of solid supports is a plurality of silicon carbide particles. In some embodiments, the whole urine is from a urine sample of a male human subject who has a prostate and who did not have a digital rectal examination (DRE) within about 180 minutes before providing the urine sample. In some embodiments, the method does not comprise admixing whole urine and the plurality of solid supports under reduced pressure. In some embodiments, the reduced pressure is about 0.8 bar. In some embodiments, the lysis buffer comprises guanidinium thiocyanate, guanidinium chloride or β-mercaptoethanol. In some embodiments, the lysis buffer comprises guanidinium thiocyanate and β-mercaptoethanol.

Further provided herein are methods for detecting in urine from a urine sample provided by a subject RNA encoded by a gene of the subject, the methods comprising:
allowing about 5 mL to about 10 mL of the urine from the urine sample to contact a silicon-based solid support to provide a urine admixture comprising an RNA-bound silicon-based solid support; isolating the RNA-bound silicon-based solid support from the urine admixture to provide an isolated RNA-bound silicone-based solid support;
isolating the RNA from the isolated RNA-bound silicon-based solid support to provide isolated RNA; and
detecting the isolated RNA,
wherein the subject has not had a digital rectal examination (DRE) within about 180 minutes before providing the urine sample.

### II. Detecting Extracted RNA

In some embodiments, the methods described herein further comprise detecting the extracted RNA. In some embodiments, the extracted RNA comprises or is miRNA (or pre-miRNA), siRNA, circular RNA, long non-coding RNA (lncRNA), piRNA, mRNA, rRNA, tRNA, hnRNA, or noncoding RNA (ncRNA). In some embodiments, the extracted RNA is mRNA. In some embodiments, the extracted RNA is miRNA. In some embodiments, the extracted RNA is siRNA. In some embodiments, the extracted RNA is circular RNA. In some embodiments, the extracted RNA is lncRNA. In some embodiments, the extracted RNA is piRNA. In some embodiments, the extracted RNA is rRNA. In some embodiments, the extracted RNA is tRNA. In some embodiments, the extracted RNA is hnRNA. In some embodiments, the extracted RNA is ncRNA.

### Detecting Gene Expression

In some embodiments, detecting the extracted RNA comprises detecting an amount of expression of the subject's gene. In some embodiments, detecting the extracted RNA comprises detecting an amount of expression of RNA transcribed from the subject's gene. In some embodiments, detecting the extracted RNA comprises detecting the amount of mRNA transcribed from the gene. In some embodiments, detecting the extracted RNA comprises reverse transcribing the extracted RNA to complementary DNA (cDNA) using reverse transcriptase and detecting the cDNA. In some embodiments, detecting the extracted RNA comprises reverse transcribing the extracted RNA to cDNA using reverse transcriptase and detecting the cDNA using quantitative PCR (qPCR).

In some embodiments, the gene is a cancer gene, e.g., a prostate cancer gene, a bladder cancer gene, or a kidney cancer gene.

In some embodiments, the gene is a gene of a pathogen, e.g., a sexually transmitted infection (STI) pathogen or a urinary tract infection (UTI) pathogen. In some embodiments, the pathogen is a bacterium. In some embodiments, the bacterium is of the genus Escherichia, Klebsiella, Proteus, Enterococcus, Staphylococcus, Pseudomonas, Staphylococcus, Streptococcus, or Staphylococcus. In some embodiments, the bacterium is Escherichia coli, Klebsiella pneumoniae, Proteus mirabilis, Enterococcus faecalis, Staphylococcus saprophyticus, Pseudomonas aeruginosa, Staphylococcus saprophyticus, Streptococcus agalactiae or Staphylococcus aureus. In some embodiments, the pathogen is a yeast. In some embodiments, the yeast is of the genus Candida. In some embodiments, the yeast is Candida albicans. In some embodiments, the gene is a gene of a pathogen and the gene is 16S rRNA. In some embodiments, the gene is of a pathogen and the gene is 23S rRNA. In some embodiments, the gene is of a pathogen and the gene is 26S rRNA. In some embodiments, the gene is of the subject's immune response to the pathogen, e.g., a gene of the subject's adaptive (B cell or T cell) or innate (neutrophil) immune response to the pathogen.

In some embodiments, the gene is a prostate cancer gene.

In some embodiments, the prostate cancer gene is KLK3.

In some embodiments, the prostate cancer gene is one or more of ACSM1, AMACR, AR, COL9A2, CRISP3, CST2, DLX1, ETV1, F5, GDF15, GLYATL1, GOLM1, GRIN3A, LINC00993, LRRN1, MIPEP, MS4A8, MYO6, PCA3.1, PDLIM5, PEX10, PLA1A, PLA2G7, PRCAT47, SPINK1, TDO2, TK1, TMEFF2, TRGV9.1, VSTM2L, TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, HOXC6, and KLK3.

In some embodiments, the prostate cancer gene is one or more of ACSM1, AMACR, AR, COL9A2, CRISP3, CST2, DLX1, ETV1, F5, GDF15, GLYATL1, GOLM1, GRIN3A, LINC00993, LRRN1, MIPEP, MS4A8, MYO6, PCA3.1, PDLIM5, PEX10, PLA1A, PLA2G7, PRCAT47, SPINK1, TDO2, TK1, TMEFF2, TRGV9.1, and VSTM2L.

In some embodiments, the prostate cancer gene is one or more of TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, HOXC6, and KLK3.

In some embodiments, the methods described herein comprise detecting an amount of expression of a TMPRSS2-ERG gene. In some embodiments, the TMPRSS2-ERG fusion comprises exon 1 of TMPRSS2 and exons 4-11 of ERG. In some embodiments, the TMPRSS2-ERG gene fusion comprises a fusion of the nucleotide sequences of Ensembl gene identifiers ENSG00000184012 and ENSG00000157554. In some embodiments, the TMPRSS2-ERG gene fusion comprises the nucleotide sequence of SEQ ID NO:1 or a variant thereof.

In some embodiments, the methods described herein comprise detecting an amount of expression of a SCHLAP1 gene. In some embodiments, the SCHLAP1 gene comprises the nucleotide sequence provided by the HUGO Gene Nomenclature Committee (HGNC). In some embodiments, the HGNC identifier for SCHLAP1 is 48603. In some embodiments, the SCHLAP1 gene is located at chromosome position 2q31.3. In some embodiments, a SCHLAP1 gene comprises the nucleotide sequence of Ensembl gene identifier ENSG00000281131. In some embodiments, a SCHLAP1 gene comprises the nucleotide sequence of SEQ ID NO:2 or a variant thereof.

In some embodiments, the methods described herein comprise detecting an amount of expression of a OR51E2 gene. In some embodiments, the OR51E2 gene comprises the nucleotide sequence provided by HGNC. In some embodiments, the HGNC identifier for OR51E2 is 15195. In some embodiments, the OR51E2 gene is located at chromosome position 11p15.4. In some embodiments, an OR51E2 gene comprises the nucleotide sequence of Ensembl gene identifier ENSG00000167332. In some embodiments, an OR51E2 gene comprises the nucleotide sequence of SEQ ID NO:3 or a variant thereof.

In some embodiments, the methods described herein comprise detecting an amount of expression of an APOC1 gene. In some embodiments, the APOC1 gene comprises the nucleotide sequence provided by HGNC. In some embodiments, the HGNC identifier for APOC1 is 607. In some embodiments, the APOC1 gene is located at chromosome position 19q13.32. In some embodiments, an APOC1 gene comprises the nucleotide sequence of Ensembl gene identifier ENSG00000130208. In some embodiments, an APOC1 gene comprises the nucleotide sequence of SEQ ID NO:4 or a variant thereof.

In some embodiments, the methods described herein comprise detecting an amount of expression of a PCAT14 gene. In some embodiments, the PCAT14 gene comprises the nucleotide sequence provided by HGNC. In some embodiments, the HGNC identifier for PCAT14 is 48977. In some embodiments, the PCAT14 gene is located at chromosome position 22q11.23. In some embodiments, a PCAT14 gene comprises the nucleotide sequence of Ensembl gene identifier ENSG00000280623. In some embodiments, a PCAT14 gene comprises the nucleotide sequence of SEQ ID NO:5 or a variant thereof.

In some embodiments, the methods described herein comprise detecting an amount of expression of a CAMKK2 gene. In some embodiments, the CAMKK2 gene comprises the nucleotide sequence provided by HGNC. In some embodiments, the HGNC identifier for CAMKK2 is 1470. In some embodiments, the CAMKK2 gene is located at chromosome position 12q24.31. In some embodiments, a CAMKK2 gene comprises the nucleotide sequence of Ensembl gene ENSG00000110931. In some embodiments, a CAMKK2 gene comprises the nucleotide sequence of SEQ ID NO:6 or a variant thereof.

In some embodiments, the methods described herein comprise detecting an amount of expression of a PCA3 gene. In some embodiments, the PCA3 gene comprises the nucleotide sequence provided by HGNC. In some embodiments, the HGNC identifier for PCA3 is 8637. In some embodiments, the PCA3 gene is located at chromosome position 9q21.2. In some embodiments, a PCA3 gene comprises the nucleotide sequence of Ensembl gene identifier ENSG00000225937. In some embodiments, a PCA3 gene comprises the nucleotide sequence of SEQ ID NO:7 or a variant thereof. In some embodiments, the method comprises detecting an amount of PCA3 at its 3′ end, wherein the 3′ end of PCA3 is referred to herein as “3′ PCA3” or “PCA3.1.” For example, and without limitation, the method can comprise detecting 3′ PCA3 at one or both of its exon groups 2-3 and 3-4. In some embodiments, the methods comprise detecting an amount of expression of PCA3 at its 5′ end, wherein the 5′ end of PCA3 is referred to herein as “5′ PCA3” or “PCA3.” For example, and without limitation, the methods can comprise detecting 5′ PCA3 at its exon group 1-2.

In some embodiments, the methods described herein comprise detecting an amount of expression of an NKAIN1 gene. In some embodiments, the NKAIN1 gene comprises the nucleotide sequence provided by HGNC. In some embodiments, the HGNC identifier for NKAIN1 is 25743. In some embodiments, the NKAIN1 gene is located at chromosome position 1p35.2. In some embodiments, an NKAIN1 gene comprises the nucleotide sequence of Ensembl gene identifier ENSG00000084628. In some embodiments, an NKAIN1 gene comprises the nucleotide sequence of SEQ ID NO:8 or a variant thereof.

In some embodiments, the methods described herein comprise detecting an amount of expression of a B3GNT6 gene. In some embodiments, the B3GNT6 gene comprises the nucleotide sequence provided by HGNC. In some embodiments, the HGNC identifier for B3GNT6 is 24141. In some embodiments, the B3GNT6 gene is located at chromosome position 11q13.5. In some embodiments, a B3GNT6 gene comprises the nucleotide sequence of Ensembl gene identifier ENSG00000198488. In some embodiments, a B3GNT6 gene comprises the nucleotide sequence of SEQ ID NO:9 or a variant thereof.

In some embodiments, the methods described herein comprise detecting an amount of expression of a TFF3 gene. In some embodiments, the TFF3 gene comprises the nucleotide sequence provided by HGNC. In some embodiments, the HGNC identifier for TFF3 is 11757. In some embodiments, the TFF3 gene is located at chromosome position 2122.3. In some embodiments, a TFF3 gene comprises the nucleotide sequence of Ensembl gene identifier ENSG00000160180. In some embodiments, a TFF3 gene comprises the nucleotide sequence of SEQ ID NO:10 or a variant thereof.

In some embodiments, the methods described herein comprise detecting an amount of expression of a SPON2 gene. In some embodiments, the SPON2 gene comprises the nucleotide sequence provided by HGNC. In some embodiments, the HGNC identifier for SPON2 is 11253. In some embodiments, the SPON2 gene is located at chromosome position 4p16.3. In some embodiments, a SPON2 gene comprises the nucleotide sequence of Ensembl gene identifier ENSG00000159674. In some embodiments, a SPON2 gene comprises the nucleotide sequence of SEQ ID NO:11 or a variant thereof.

In some embodiments, the methods described herein comprise detecting an amount of expression of a PCGEM1 gene. In some embodiments, the PCGEM1 gene comprises the nucleotide sequence provided by HGNC. In some embodiments, the HGNC identifier for PCGEM1 is 30145. In some embodiments, the PCGEM1 gene is located at chromosome position 2q32.3. In some embodiments, a PCGEM1 gene comprises the nucleotide sequence of Ensembl gene identifier ENSG00000227418. In some embodiments, a PCGEM1 gene comprises the nucleotide sequence of SEQ ID NO:12 or a variant thereof.

In some embodiments, the methods described herein comprise detecting an amount of expression of a TRGV9 gene. In some embodiments, the TRGV9 gene comprises the nucleotide sequence provided by HGNC. In some embodiments, the HGNC identifier for TRGV9 is 12295. In some embodiments, the TRGV9 gene is located at chromosome position 7p14.1. In some embodiments, a TRGV9 gene comprises the nucleotide sequence of Ensembl gene identifier ENSG00000211695. In some embodiments, a TRGV9 gene comprises the nucleotide sequence of SEQ ID NO:13 or a variant thereof. In some embodiments, the methods comprise detecting an amount of expression of TRGV9 at its 3′ end, wherein the 3′ end of TRGV9 is referred to herein as “3′ TRGV9.” For example, and without limitation, the methods can comprise detecting 3′ TRGV9 at one or more of its exon groups 3-4, 4-5, and 5-6. In some embodiments, the methods comprise detecting an amount of expression of TRGV9 at its 5′ end, wherein the 5′ end of TRGV9 is referred to herein as “5′ TRGV9.” For example, and without limitation, the methods can comprise detecting 5′ TRGV9 at its exon group 1-2.

In some embodiments, the methods described herein comprise detecting an amount of expression of a TMSB15A gene. In some embodiments, the TMSB15A gene comprises the nucleotide sequence provided by HGNC. In some embodiments, the HGNC identifier for TMSB15A is 30744. In some embodiments, the TMSB15A gene is located at chromosome position Xq22.1. In some embodiments, a TMSB15A gene comprises the nucleotide sequence of Ensembl gene identifier ENSG00000158164. In some embodiments, a TMSB15A gene comprises the nucleotide sequence of SEQ ID NO:14 or a variant thereof.

In some embodiments, the methods described herein comprise detecting an amount of expression of an ERG gene. In some embodiments, the ERG gene comprises the nucleotide sequence provided by HGNC. In some embodiments, the HGNC identifier for ERG is 3446. In some embodiments, the ERG gene is located at chromosome position 2122.2. In some embodiments, an ERG gene comprises the nucleotide sequence of Ensembl gene identifier ENSG00000157554. In some embodiments, an ERG gene comprises the nucleotide sequence of SEQ ID NO:15 or a variant thereof.

In some embodiments, the methods described herein comprise detecting an amount of expression of a KLK4 gene. In some embodiments, the KLK4 gene comprises the nucleotide sequence provided by HGNC. In some embodiments, the HGNC identifier for KLK4 is 6365. In some embodiments, the KLK4 gene is located at chromosome position 19q13.41. In some embodiments, a KLK4 gene comprises the nucleotide sequence of Ensembl gene identifier ENSG00000167749. In some embodiments, a KLK4 gene comprises the nucleotide sequence of SEQ ID NO:16 or a variant thereof.

In some embodiments, the methods described herein comprise detecting an amount of expression of a HOXC6 gene. In some embodiments, the HOXC6 gene comprises the nucleotide sequence provided by HGNC. In some embodiments, the HGNC identifier for HOXC6 is 5128. In some embodiments, the HOXC6 gene is located at chromosome position 12q13.13. In some embodiments, a HOXC6 gene comprises the nucleotide sequence of Ensembl gene identifier ENSG00000197757. In some embodiments, a HOXC6 gene comprises the nucleotide sequence of SEQ ID NO:17 or a variant thereof.

In some embodiments, the methods described herein comprise detecting an amount of expression of a KLK3 gene. KLK3 may also be known as PSA or Prostate-Specific Antigen. In some embodiments, the KLK3 gene comprises the nucleotide sequence provided by HGNC. In some embodiments, the HGNC identifier for KLK3 is 6364. In some embodiments, the KLK3 gene is located at chromosome position 19q13.33. In some embodiments, a KLK3 gene comprises the nucleotide sequence of Ensembl gene identifier ENSG00000142515. In some embodiments, a KLK3 gene comprises the nucleotide sequence of SEQ ID NO:18 or a variant thereof.

In some embodiments, the methods described herein comprise detecting an amount of expression of an ACSM1 gene. In some embodiments, the ACSM1 gene comprises the nucleotide sequence provided by HGNC. In some embodiments, the HGNC identifier for ACSM1 is 18049. In some embodiments, the ACSM1 gene is located at chromosome position 16p12.3. In some embodiments, an ACSM1 gene comprises the nucleotide sequence of Ensembl gene identifier ENSG00000166743. In some embodiments, an ACSM1 gene comprises the nucleotide sequence of SEQ ID NO:19 or a variant thereof.

In some embodiments, the methods described herein comprise detecting an amount of expression of an AMACR gene. In some embodiments, the AMACR gene comprises the nucleotide sequence provided by HGNC. In some embodiments, the HGNC identifier for AMACR is 451. In some embodiments, the AMACR gene is located at chromosome position 5p13.2. In some embodiments, an AMACR gene comprises the nucleotide sequence of Ensembl gene identifier ENSG00000242110. In some embodiments, an AMACR gene comprises the nucleotide sequence of SEQ ID NO:20 or a variant thereof.

In some embodiments, the methods described herein comprise detecting an amount of expression of an AR gene. In some embodiments, the AR gene comprises the nucleotide sequence provided by HGNC. In some embodiments, the HGNC identifier for AR is 644. In some embodiments, the AR gene is located at chromosome position Xq12. In some embodiments, an AR gene comprises the nucleotide sequence of Ensembl gene identifier ENSG00000169083. In some embodiments, an AR gene comprises the nucleotide sequence of SEQ ID NO:21 or a variant thereof.

In some embodiments, the methods described herein comprise detecting an amount of expression of a COL9A2 gene. In some embodiments, the COL9A2 gene comprises the nucleotide sequence provided by HGNC. In some embodiments, the HGNC identifier for COL9A2 is 2218. In some embodiments, the COL9A2 gene is located at chromosome position 1p34.2. In some embodiments, a COL9A2 gene comprises the nucleotide sequence of Ensembl gene identifier ENSG00000049089. In some embodiments, an COL9A2 gene comprises the nucleotide sequence of SEQ ID NO:22 or a variant thereof.

In some embodiments, the methods described herein comprise detecting an amount of expression of a CRISP3 gene. In some embodiments, the CRISP3 gene comprises the nucleotide sequence provided by HGNC. In some embodiments, the HGNC identifier for CRISP3 is 16904. In some embodiments, the CRISP3 gene is located at chromosome position 6p12.3. In some embodiments, a CRISP3 gene comprises the nucleotide sequence of Ensembl gene identifier ENSG00000096006. In some embodiments, a CRISP3 gene comprises the nucleotide sequence of SEQ ID NO:23 or a variant thereof.

In some embodiments, the methods described herein comprise detecting an amount of expression of a CST2 gene. In some embodiments, the CST2 gene comprises the nucleotide sequence provided by HGNC. In some embodiments, the HGNC identifier for CST2 is 2474. In some embodiments, the CST2 gene is located at chromosome position 20p11.21. In some embodiments, a CST2 gene comprises the nucleotide sequence of Ensembl gene identifier ENSG00000170369. In some embodiments, a CST2 gene comprises the nucleotide sequence of SEQ ID NO:24 or a variant thereof.

In some embodiments, the methods described herein comprise detecting an amount of expression of a DLX1 gene. In some embodiments, the DLX1 gene comprises the nucleotide sequence provided by HGNC. In some embodiments, the HGNC identifier for DLX1 is 2914. In some embodiments, the DLX1 gene is located at chromosome position 2q31.1. In some embodiments, a DLX1 gene comprises the nucleotide sequence of Ensembl gene identifier ENSG00000144355. In some embodiments, a DLX1 gene comprises the nucleotide sequence of SEQ ID NO:25 or a variant thereof.

In some embodiments, the methods described herein comprise detecting an amount of expression of an ETV1 gene. In some embodiments, the ETV1 gene comprises the nucleotide sequence provided by HGNC. In some embodiments, the HGNC identifier for ETV1 is 3490. In some embodiments, the ETV1 gene is located at chromosome position 7p21.2. In some embodiments, an ETV1 gene comprises the nucleotide sequence of Ensembl gene identifier ENSG00000006468. In some embodiments, an ETV1 gene comprises the nucleotide sequence of SEQ ID NO:26 or a variant thereof.

In some embodiments, the methods described herein comprise detecting an amount of expression of an F5 gene. In some embodiments, the F5 gene comprises the nucleotide sequence provided by HGNC. In some embodiments, the HGNC identifier for F5 is 3542. In some embodiments, the F5 gene is located at chromosome position 1q24.2. In some embodiments, an F5 gene comprises the nucleotide sequence of Ensembl gene identifier ENSG00000198734. In some embodiments, an F5 gene comprises the nucleotide sequence of SEQ ID NO:27 or a variant thereof.

In some embodiments, the methods described herein comprise detecting an amount of expression of a GDF15 gene. In some embodiments, the GDF15 gene comprises the nucleotide sequence provided by HGNC. In some embodiments, the HGNC identifier for GDF15 is 30142. In some embodiments, the GDF15 gene is located at chromosome position 19p13.11. In some embodiments, a GDF15 gene comprises the nucleotide sequence of Ensembl gene identifier ENSG00000130513. In some embodiments, a GDF15 gene comprises the nucleotide sequence of SEQ ID NO:28 or a variant thereof.

In some embodiments, the methods described herein comprise detecting an amount of expression of a GLYATL1 gene. In some embodiments, the GLYATL1 gene comprises the nucleotide sequence provided by HGNC. In some embodiments, the HGNC identifier for GLYATL1 is 17257. In some embodiments, the GLYATL1 gene is located at chromosome position 11q12.1. In some embodiments, a GLYATL1 gene comprises the nucleotide sequence of Ensembl gene identifier ENSG00000166840. In some embodiments, a GLYATL1 gene comprises the nucleotide sequence of SEQ ID NO:29 or a variant thereof.

In some embodiments, the methods described herein comprise detecting an amount of expression of a GOLM1 gene. In some embodiments, the GOLM1 gene comprises the nucleotide sequence provided by HGNC. In some embodiments, the HGNC identifier for SPDEF is 15451. In some embodiments, the GOLM1 gene is located at chromosome position 9q21.33. In some embodiments, a GOLM1 gene comprises the nucleotide sequence of Ensembl gene identifier ENSG00000135052. In some embodiments, a GOLM1 gene comprises the nucleotide sequence of SEQ ID NO:30 or a variant thereof.

In some embodiments, the methods described herein comprise detecting an amount of expression of a GRIN3A gene. In some embodiments, the GRIN3A gene comprises the nucleotide sequence provided by HGNC. In some embodiments, the HGNC identifier for GRIN3A is 16767. In some embodiments, the GRIN3A gene is located at chromosome position 9q31.1. In some embodiments, a GRIN3A gene comprises the nucleotide sequence of Ensembl gene identifier ENSG00000198785. In some embodiments, an GRIN3A gene comprises the nucleotide sequence of SEQ ID NO:31 or a variant thereof.

In some embodiments, the methods described herein comprise detecting an amount of expression of a LINC00993 gene. In some embodiments, the LINC00993 gene comprises the nucleotide sequence provided by HGNC. In some embodiments, the HGNC identifier for LINC00993 is 48948. In some embodiments, the LINC00993 gene is located at chromosome position 10p11.21. In some embodiments, a LINC00993 gene comprises the nucleotide sequence of Ensembl gene identifier ENSG00000235687. In some embodiments, a LINC00993 gene comprises the nucleotide sequence of SEQ ID NO:32 or a variant thereof.

In some embodiments, the methods described herein comprise detecting an amount of expression of a LRRN1 gene. In some embodiments, the LRRN1 gene comprises the nucleotide sequence provided by HGNC. In some embodiments, the HGNC identifier for LRRN1 is 20980. In some embodiments, the LRRN1 gene is located at chromosome position 3p26.2. In some embodiments, a LRRN1 gene comprises the nucleotide sequence of Ensembl gene identifier ENSG00000175928. In some embodiments, an LRRN1 gene comprises the nucleotide sequence of SEQ ID NO:33 or a variant thereof.

In some embodiments, the methods described herein comprise detecting an amount of expression of a MIPEP gene. In some embodiments, the MIPEP gene comprises the nucleotide sequence provided by HGNC. In some embodiments, the HGNC identifier for MIPEP is 7104. In some embodiments, the MIPEP gene is located at chromosome position 13q12.12. In some embodiments, a MIPEP gene comprises the nucleotide sequence of Ensembl gene identifier ENSG00000027003. In some embodiments, a MIPEP gene comprises the nucleotide sequence of SEQ ID NO:34 or a variant thereof.

In some embodiments, the methods described herein comprise detecting an amount of expression of an MS4A8 gene. In some embodiments, the MS4A8 gene comprises the nucleotide sequence provided by HGNC. In some embodiments, the HGNC identifier for MS4A8 is 13380. In some embodiments, the MS4A8 gene is located at chromosome position 11q12.2. In some embodiments, an MS4A8 gene comprises the nucleotide sequence of Ensembl gene identifier ENSG00000166959. In some embodiments, an MS4A8 gene comprises the nucleotide sequence of SEQ ID NO:35 or a variant thereof.

In some embodiments, the methods described herein comprise detecting an amount of expression of a MYO6 gene. In some embodiments, the MYO6 gene comprises the nucleotide sequence provided by HGNC. In some embodiments, the HGNC identifier for MYO6 is 7605. In some embodiments, the MYO6 gene is located at chromosome position 6914.1. In some embodiments, a MYO6 gene comprises the nucleotide sequence of Ensembl gene identifier ENSG00000196586. In some embodiments, a MYO6 gene comprises the nucleotide sequence of SEQ ID NO:36 or a variant thereof.

In some embodiments, the methods described herein comprise detecting an amount of expression of a CYB561A3 gene. In some embodiments, the CYB561A3 gene comprises the nucleotide sequence provided by HGNC. In some embodiments, the HGNC identifier for CYB561A3 is 23014. In some embodiments, the CYB561A3 gene is located at chromosome position 11q12.2. In some embodiments, a CYB561A3 gene comprises the nucleotide sequence of Ensembl gene identifier ENSG00000162144. In some embodiments, a CYB561A3 gene comprises the nucleotide sequence of SEQ ID NO:37 or a variant thereof.

In some embodiments, the methods described herein comprise detecting an amount of expression of a PDLIM5 gene. In some embodiments, the PDLIM5 gene comprises the nucleotide sequence provided by HGNC. In some embodiments, the HGNC identifier for PDLIM5 is 17468. In some embodiments, the PDLIM5 gene is located at chromosome position 4q22.3. In some embodiments, a PDLIM5 gene comprises the nucleotide sequence of Ensembl gene identifier ENSG00000163110. In some embodiments, a PDLIM5 gene comprises the nucleotide sequence of SEQ ID NO:38 or a variant thereof.

In some embodiments, the methods described herein comprise detecting an amount of expression of a PEX10 gene. In some embodiments, the PEX10 gene comprises the nucleotide sequence provided by HGNC. In some embodiments, the HGNC identifier for PEX10 is 8851. In some embodiments, the PEX10 gene is located at chromosome position 1p36.32. In some embodiments, a PEX10 gene comprises the nucleotide sequence of Ensembl gene identifier ENSG00000157911. In some embodiments, a PEX10 gene comprises the nucleotide sequence of SEQ ID NO:39 or a variant thereof.

In some embodiments, the methods described herein comprise detecting an amount of expression of a PLA1A gene. In some embodiments, the PLA1A gene comprises the nucleotide sequence provided by HGNC. In some embodiments, the HGNC identifier for PLA1A is 17661. In some embodiments, the PLA1A gene is located at chromosome position 3q13.33. In some embodiments, a PLA1A gene comprises the nucleotide sequence of Ensembl gene identifier ENSG00000144837. In some embodiments, a PLA1A gene comprises the nucleotide sequence of SEQ ID NO:40 or a variant thereof.

In some embodiments, the methods described herein comprise detecting an amount of expression of a PLA2G7 gene. In some embodiments, the PLA2G7 gene comprises the nucleotide sequence provided by HGNC. In some embodiments, the HGNC identifier for PLA2G7 is 9040. In some embodiments, the PLA2G7 gene is located at chromosome position 6p12.3. In some embodiments, a PLA2G7 gene comprises the nucleotide sequence of Ensembl gene identifier ENSG00000146070. In some embodiments, a PLA2G7 gene comprises the nucleotide sequence of SEQ ID NO:41 or a variant thereof.

In some embodiments, the methods described herein comprise detecting an amount of expression of a PRCAT47 gene. In some embodiments, the PRCAT47 gene comprises the nucleotide sequence provided by HGNC. In some embodiments, the HGNC identifier for PRCAT47 is 53032. In some embodiments, the PRCAT47 gene is located at chromosome position 16q23.2. In some embodiments, a PRCAT47 gene comprises the nucleotide sequence of Ensembl gene identifier ENSG00000260896. In some embodiments, a PRCAT47 gene comprises the nucleotide sequence of SEQ ID NO:42 or a variant thereof.

In some embodiments, the methods described herein comprise detecting an amount of expression of a SPINK1 gene. In some embodiments, the SPINK1 gene comprises the nucleotide sequence provided by HGNC. In some embodiments, the HGNC identifier for SPINK1 is 11244. In some embodiments, the SPINK1 gene is located at chromosome position 38.p14. In some embodiments, a SPINK1 gene comprises the nucleotide sequence of Ensembl gene identifier ENSG00000164266. In some embodiments, a SPINK1 gene comprises the nucleotide sequence of SEQ ID NO:43 or a variant thereof.

In some embodiments, the methods described herein comprise detecting an amount of expression of a TDO2 gene. In some embodiments, the TDO2 gene comprises the nucleotide sequence provided by HGNC. In some embodiments, the HGNC identifier for TDO2 is 11708. In some embodiments, the TDO2 gene is located at chromosome position 4q32.1. In some embodiments, a TDO2 gene comprises the nucleotide sequence of Ensembl gene identifier ENSG00000151790. In some embodiments, an TDO2 gene comprises the nucleotide sequence of SEQ ID NO:44 or a variant thereof.

In some embodiments, the methods described herein comprise detecting an amount of expression of a TK1 gene. In some embodiments, the TK1 gene comprises the nucleotide sequence provided by HGNC. In some embodiments, the HGNC identifier for TK1 is 11830. In some embodiments, the TK1 gene is located at chromosome position 17q25.3. In some embodiments, a TK1 gene comprises the nucleotide sequence of Ensembl gene identifier ENSG00000167900. In some embodiments, a TK1 gene comprises the nucleotide sequence of SEQ ID NO:45 or a variant thereof.

In some embodiments, the methods described herein comprise detecting an amount of expression of a TMEFF2 gene. In some embodiments, the TMEFF2 gene comprises the nucleotide sequence provided by HGNC. In some embodiments, the HGNC identifier for TMEFF2 is 11867. In some embodiments, the TMEFF2 gene is located at chromosome position 2q32.3. In some embodiments, a TMEFF2 gene comprises the nucleotide sequence of Ensembl gene identifier ENSG00000144339. In some embodiments, a TMEFF2 gene comprises the nucleotide sequence of SEQ ID NO:46 or a variant thereof.

In some embodiments, the methods described herein comprise detecting an amount of expression of a VSTM2L gene. In some embodiments, the VSTM2L gene comprises the nucleotide sequence provided by HGNC. In some embodiments, the HGNC identifier for VSTM2L is 16096. In some embodiments, the VSTM2L gene is located at chromosome position 20q11.23. In some embodiments, a VSTM2L gene comprises the nucleotide sequence of Ensembl gene identifier ENSG00000132821. In some embodiments, a VSTM2L gene comprises the nucleotide sequence of SEQ ID NO:47 or a variant thereof.

In some embodiments, the methods described herein comprise detecting an amount of expression of a KLK2 gene. In some embodiments, the KLK2 gene comprises the nucleotide sequence provided by HGNC. In some embodiments, the HGNC identifier for KLK2 is 6363. In some embodiments, the KLK2 gene is located at chromosome position 19q13.33. In some embodiments, a KLK2 gene comprises the nucleotide sequence of Ensembl gene identifier ENSG00000167751. In some embodiments, a KLK2 gene comprises the nucleotide sequence of SEQ ID NO:48 or a variant thereof.

In some embodiments, the methods described herein comprise detecting an amount of expression of a NUDT8 gene. In some embodiments, the NUDT8 gene comprises the nucleotide sequence provided by HGNC. In some embodiments, the HGNC identifier for NUDT8 is 8055. In some embodiments, the NUDT8 gene is located at chromosome position 11q13.2. In some embodiments, a NUDT8 gene comprises the nucleotide sequence of Ensembl gene identifier ENSG00000167799. In some embodiments, a NUDT8 gene comprises the nucleotide sequence of SEQ ID NO:49 or a variant thereof.

In some embodiments, the methods described herein comprise detecting an amount of expression of an EEF1A2 gene. In some embodiments, the EEF1A2 gene comprises the nucleotide sequence provided by HGNC. In some embodiments, the HGNC identifier for EEF1A2 is 3192. In some embodiments, the EEF1A2 gene is located at chromosome position 20q13.33. In some embodiments, an EEF1A2 gene comprises the nucleotide sequence of Ensembl gene identifier ENSG00000101210. In some embodiments, an EEF1A2 gene comprises the nucleotide sequence of SEQ ID NO:50 or a variant thereof.

In some embodiments, the methods described herein comprise detecting an amount of expression of an SPDEF gene. In some embodiments, the SPDEF gene comprises the nucleotide sequence provided by HGNC. In some embodiments, the HGNC identifier for SPDEF is 17257. In some embodiments, the SPDEF gene is located at chromosome position 6p21.31. In some embodiments, an SPDEF gene comprises the nucleotide sequence of Ensembl gene identifier ENSG00000124664. In some embodiments, an SPDEF gene comprises the nucleotide sequence of SEQ ID NO:51 or a variant thereof.

In some embodiments, the methods described herein comprise detecting an amount of expression of a GAPDH gene. In some embodiments, the GAPDH gene comprises the nucleotide sequence provided by HGNC. In some embodiments, the HGNC identifier for GAPDH is 4141. In some embodiments, the GAPDH gene is located at chromosome position 12p13.31. In some embodiments, a GAPDH gene comprises the nucleotide sequence of Ensembl gene identifier ENSG00000111640. In some embodiments, a GAPDH gene comprises the nucleotide sequence of SEQ ID NO:52 or a variant thereof.

In some embodiments, the methods described herein comprise detecting an amount of expression of a LBH gene. In some embodiments, the LBH gene comprises the nucleotide sequence provided by HGNC. In some embodiments, the HGNC identifier for LBH is 29532. In some embodiments, the LBH gene is located at chromosome position 2p23.1. In some embodiments, a LBH gene comprises the nucleotide sequence of Ensembl gene identifier ENSG00000213626. In some embodiments, a LBH gene comprises the nucleotide sequence of SEQ ID NO:53 or a variant thereof.

In some embodiments, the methods described herein comprise detecting an amount of expression of an HPN gene. In some embodiments, the HPN gene comprises the nucleotide sequence provided by HGNC. In some embodiments, the HGNC identifier for HPN is 5155. In some embodiments, the HPN gene is located at chromosome position 19q13.11. In some embodiments, an HPN gene comprises the nucleotide sequence of Ensembl gene identifier ENSG00000105707. In some embodiments, an HPN gene comprises the nucleotide sequence of SEQ ID NO:54 or a variant thereof.

Sequence(s) of a subject's gene disclosed herein are available from publicly available gene sequence databases, including but not limited to the Ensembl Genome Browser (available at https://www.ensembl.org/index.html).

Nucleotide sequences of illustrative genes of the subject are provided in Table 1.

TABLE 1
Illustrative nucleotide sequences of genes of the subject.

| SEQ ID | | |
|---|---|---|
| NO: | Gene | Sequence |
| 1 | TMPRSS2- | TAGGCGCGAG CTAAGCAGGA GGCGGAGGCG GAGGCGGAGG GCGAGGGGCG |
| | ERG | GGGAGCGCCG CCTGGAGCGC GGCAGGAAGC CTTATCAGTT GTGAGTGAGG |
| | | ACCAGTCGTT GTTTGAGTGT GCCTACGGAA CGCCACACCT GGCTAAGACA |
| | | GAGATGACCG CGTCCTCCTC CAGCGACTAT GGACAGACTT CCAAGATGAG |
| | | CCCACGCGTC CCTCAGCAGG ATTGGCTGTC TCAACCCCCA GCCAGGGTCA |
| | | CCATCAAAAT GGAATGTAAC CCTAGCCAGG TGAATGGCTC AAG |
| 2 | SCHLAP1 | GCTTTTATGA GCTGTAACAC TCACCGCGAA GGTCCGCAGC TTCACTCCTG |
| | | AAGCCAGCGA GACCACGAGC CTACTGGGAG GAACGAACAA CTCCCGACGC |
| | | GCCGCCTTAA GAGCTGTAAC ACTCACCGCG AAGGTCTGCA GCTTCACTCC |
| | | TGAGCCAGCG AGACCACGAA CCCACCAGAA GGAAAAAACT CCGAACACAT |
| | | CTGAACATCA GAAGCAACAA ACTCCGGACA CGCCGCCTTT AAGAACTGTA |
| | | ACACTCACTG CGAGGGTCCG CGGCTTCATT CTTGAAGTGA GTGAGACCAA |
| | | GAACCCACCA GTTCTGGACA CAATTTCAAG TCCTCAGGTG CCATCAATAT |
| | | TCTGAAAATG GCAGTGATTT TTATTCAACC TGTATAAGGC ACTTTCACCA |
| | | TGTACCTGGA AGCAACATCT ACATCTTTTT CAGTTTCTTC TACGCCAGGT |
| | | GTGTGCTTAG CTCCATGACA AAAGGTGACA GCTTATTCTG CAGCACACAC |
| | | ACATCATCAA AGTGGGAGGT GGTGAGACTG GCACACTGAC AGTCTGTCCT |
| | | AGCAGATTTC AGCTCACACT GCAATCTAGA TGCTGGGGAC ACAAGGTCCA |
| | | CCTTCCAGGA ATATGGCCAT GACACCAGAA ATCACAAACA TGATGAGAAT |
| | | GGAATGACTG GGGAAGAAGT GCCAGATGCT TCACTTGTAA ATGAAGACCC |
| | | AGCCTCTGGG GATGCAGATA CCACCTCCCT GAAGAAGCTG AATATCTGCA |
| | | GATAAGTGGA GTTCACCAAT GATGAGGAGC GGGATGGAGA AAGGAGGTAG |
| | | GGAGAGTCAT CCAAGGAACA TGAGCAACAT GTTAAAAGCC AAGTGGTTTA |
| | | ATTTCTGGAG ATGGTGAACC CAAGAGGCTC TGCTGGGAGA CAACAAAAAT |
| | | AATGAAGAAT TGAACCAGAG TCCGGTGAAT ATCAGCACTG GGACCAGTTA |
| | | GCAGAGGAAA AGGAAAGAAT AAAAGCGAAA AGAATGAAGA GTCATATGAT |
| | | TACCAACTTT TCCTTTTTCA TATAAATTGA GTGTATATGG GTCTGGAACA |
| | | ACCTGAATTT CCATCAAGTC CTGGCTAACC TCATTATGTC CTATGAATAT |
| | | TTTTGACTAA TCCCACTTTA CATTAATCTG TATTGTGAAT GTGGATATTG |
| | | AATTATATTT CTTTGTAATC CCATTATCCA AAATCCAGTT CAGAGACTAT |
| | | TAGTTACCAA TGTTCACTGT GAAGGAAAAA AAAAAAAAAA AAGCTCAGAG |
| | | GATAAACATG TGATATGGTT TGGCTGTGTC CCCACCCAAA TATCATCTTG |
| | | AATTGTAGCT CCCATAATTC CCACGTGTTG TGGGAGGGAC CCGGTGGGAG |
| | | ATAATTGTAT CATGGGGGTG GTTCCCCCAT ACTATTCTCA TAGTAGTGAA |
| | | TAAGTCTCAC AAAATCTGAT GGTTTTATGA GGGAAAACCC CTTTCACCTG |
| | | GTTCTCATTC TCTTCTCTGG TCTGTCGTCA TGTAAGACAT GCCTTTCACC |
| | | TTCTCCACCA TGACTGTGAG GCCTCCCCAG CCACGTGGAA CTGTGAGCCC |
| | | ATTAAACCTC TTTCACTTAT AAAT |
| 3 | OR51E2 | CTTCTGGGAA TCTCCACACC CTGAAGACAC AGTGAGTTAG CACCACCACC |
| | | AGGAATTGGC CITTCAGCTC TGTGCCTGTC TCCAGTCAGG CTGGAATAAG |
| | | TCTCCTCATA TTTGCAAGCT CGGCCCTCCC CTGGAATCTA AAGCCTCCTC |
| | | AGCCTTCTGA GTCAGCCTGA AAGGAACAGG CCGAACTGCT GTATGGGCTC |
| | | TACTGCCAGT GTGACCTCAC CCTCTCCAGT CACCCCTCCT CAGTTCCAGC |
| | | TATGAGTTCC TGCAACTTCA CACATGCCAC CTTTGTGCTT ATTGGTATCC |
| | | CAGGATTAGA GAAAGCCCAT TTCTGGGTTG GCTTCCCCCT CCTTTCCATG |
| | | TATGTAGTGG CAATGTTTGG AAACTGCATC GTGGTCTTCA TCGTAAGGAC |
| | | GGAACGCAGC CTGCACGCTC CGATGTACCT CTTTCTCTGC ATGCTTGCAG |
| | | CCATTGACCT GGCCTTATCC ACATCCACCA TGCCTAAGAT CCTTGCCCTT |
| | | TTCTGGTTTG ATTCCCGAGA GATTAGCTTT GAGGCCTGTC TTACCCAGAT |
| | | GTTCTTTATT CATGCCCTCT CAGCCATTGA ATCCACCATC CTGCTGGCCA |
| | | TGGCCTTTGA CCGTTATGTG GCCATCTGCC ACCCACTGCG CCATGCTGCA |
| | | GTGCTCAACA ATACAGTAAC AGCCCAGATT GGCATCGTGG CTGTGGTCCG |
| | | CGGATCCCTC TTTTTTTTCC CACTGCCTCT GCTGATCAAG CGGCTGGCCT |
| | | TCTGCCACTC CAATGTCCTC TCGCACTCCT ATTGTGTCCA CCAGGATGTA |
| | | ATGAAGTTGG CCTATGCAGA CACTTTGCCC AATGTGGTAT ATGGTCTTAC |
| | | TGCCATTCTG CTGGTCATGG GCGTGGACGT AATGTTCATC TCCTTGTCCT |
| | | ATTTTCTGAT AATACGAACG GTTCTGCAAC TGCCTTCCAA GTCAGAGCGG |
| | | GCCAAGGCCT TTGGAACCTG TGTGTCACAC ATTGGTGTGG TACTCGCCTT |
| | | CTATGTGCCA CTTATTGGCC TCTCAGTGGT ACACCGCTTT GGAAACAGCC |
| | | TTCATCCCAT TGTGCGTGTT GTCATGGGTG ACATCTACCT GCTGCTGCCT |
| | | CCTGTCATCA ATCCCATCAT CTATGGTGCC AAAACCAAAC AGATCAGAAC |
| | | ACGGGTGCTG GCTATGTTCA AGATCAGCTG TGACAAGGAC TTGCAGGCTG |
| | | TGGGAGGCAA GTGACCCTTA ACACTACACT TCTCCTTATC TTTATTGGCT |
| | | TGATAAACAT AATTATTTCT AACACTAGCT TATTTCCAGT TGCCCATAAG |
| | | CACATCAGTA CTTTTCTCTG GCTGGAATAG TAAACTAAAG TATGGTACAT |
| | | CTACCTAAAG GACTATTATG TGGAATAATA CATACTAATG AAGTATTACA |
| | | TGATTTAAAG ACTACAATAA AACCAAACAT GCTTATAACA TTAAGAAAAA |
| | | CAATAAAGAT ACATGATTGA AACCAAGTTG AAAAATAGCA TATGCCTTGG |
| | | AGGAAATGTG CTCAAATTAC TAATGATTTA GTGTTGTCCC TACTTTCTCT |
| | | CTCTTTTTTC TTTCTTTTTT TTTTATTATG GTTAGCTGTC ACATACAACT |
| | | TTTTTTTTTT TTGAGATGGG GTCTCGCTCT GTCACCAGGC TGGAGTGCAG |
| | | TGGCGCGATC TCGGCTCACT GCAACCTCCA CATCCCATGT TGAAGTAATT |
| | | CTTCTGCCTC AGCCTCCCGA GTAGCTGGGA CTAGAGGAAC GTGCCACCAT |
| | | GACTGGCTAA TTTTCTGTAT TTTTTAGTAG AGACAGAGTT TCACCATGTT |
| | | GGCCAGGATG GTCTCGATCT CCTGACCTTG TGATCCACCC GCCTCAGCCT |
| | | CCCAAAGTGT TGGGATTACA GGTGTGAACC ACTGTGCCCG GCCTGTGTAC |
| | | AACTTTTTAA ATAGGGAATA TGATAGCTTC GCATGGTGGT GTGCACCTAT |
| | | AGCCCCCACT GCCTGGAAAG CTGAGGTGGG AGAATCGCTT GAGTCCAGGA |
| | | GTTTGAGGTT ACAGTGATCC ACGATCGTAC CACTACACTC CAGCCTGGGC |
| | | AACAGAGCAA GACCCTGTCT CAAAGCATAA AATGGAATAA CATATCAAAT |
| | | GAAACAGGGA AAATGAAGCT GACAATTTAT GGAAGCCAGG GCTTGTCACA |
| | | GTCTCTACTG TTATTATGCA TTACCTGGGA ATTTATATAA GCCCTTAATA |
| | | ATAATGCCAA TGAACATCTC ATGTGTGCTC ACAATGTTCT GGCACTATTA |
| | | TAAGTGCTTC ACAGGTTTTA TGTGTTCTTC GTAACTTTAT GGAGTAGGTA |
| | | CCATTTGTGT CTCTTTATTA TAAGTGAGAG AAATGAAGTT TATATTATCA |
| | | AGGGGACTAA AGTCACACGG CTTGTGGGCA CTGTGCCAAG ATTTAAAATT |
| | | AAATTTGATG GTTGAATACA GTTACTTAAT GACCATGTTA TATTGCTTCC |
| | | TGTGTAACAT CTGCCATTTA TTTCCTCAGC TGTACAAATC CTCTGTTTTC |
| | | TCTCTGTTAC ACACTAACAT CAATGGCTTT GTACTTGTGA TGAGAGATAA |
| | | CCTTGCCCTA GTTGTGGGCA ACACATGCAG AATAATCCTG TTTTACAGCT |
| | | GCCTTTCGTG ATCTTATTGC TTGCTTTTTT CCAGATTCAG GGAGAATGTT |
| | | GTTGTCTATT TGTCTCTTAC ATCTCCTTGA TCATGTCTTC ATTTTTTAAT |
| | | GTGCTCTGTA CCTGTCAAAA ATTTTGAATG TACACCACAT GCTATTGTCT |
| | | GAACTTGAGT ATAAGATAAA ATAAAATTTT ATTTTAAATT TT |
| 4 | APOCI | AGGCGGTCAG GGGAAGGCTC AGGAGGAGGG AGATCAACAT CAACCTGCCC |
| | | CGCCCCCTCC CCAGCCTGAT AAAGGTCCTG CGGGCAGGAC AGGACCTCCC |
| | | AACCAAGCCC TCCAGCAAGG ATTCAGAGTG CCCCTCCGGC CTCGCCATGA |
| | | GGCTCTTCCT GTCGCTCCCG GTCCTGGTGG TGGTTCTGTC GATCGTCTTG |
| | | GAAGGCCCAG CCCCAGCCCA GGGGACCCCA GACGTCTCCA GTGCCTTGGA |
| | | TAAGCTGAAG GAGTTTGGAA ACACACTGGA GGACAAGGCT CGGGAACTCA |
| | | TCAGCCGCAT CAAACAGAGT GAACTTTCTG CCAAGATGCG GGAGTGGTTT |
| | | TCAGAGACAT TTCAGAAAGT GAAGGAGAAA CTCAAGATTG ACTCATGAGG |
| | | ACCTGAAGGG TGACATCCCA GGAGGGGCCT CTGAAATTTC CCACACCCCA |
| | | GCGCCTGTGC TGAGGACTCC CTCCATGTGG CCCCAGGTGC CACCAATAAA |
| | | AATCCTACAG AAAA |
| 5 | PCAT14 | GAGATACGGC CTCGTGGGAA GGGAAAGACC TGACCGTCCC CCAGCCCGAC |
| | | ACCCGTAAAG GGTCTGTGCT GAGGAGGATT AGTAAAAGGG GAAGGCCTCT |
| | | TGCAGTTGAG ATAAGAGGAA GGCCTCCGTC TCCTGCATGT CCTTGGGAAT |
| | | GGAATGTCTT GGTGTAAAAC CCGATAGTAC ATTCCTTCTA TTCTGAGAGA |
| | | AGAAAACCAC CCTGTGGCTG GAGGGTGAAG GTACTCTACA GTGTGGTCAT |
| | | TGAGGACAAG TTGACGAGAG AGTCCCAAGT ACGTCCACGG TCAGCCTTGC |
| | | GACATTTAAA GTTCTACAAT GAACTCACTG GAGATGCAAA GAAAAGTGTG |
| | | GAGATGGAGA CACCCCAATC GACTCGCCAG TCTACAGGTG TATCCAGCAG |
| | | CTCCAAAGAG ACAGCAACCA GCAAGAATGG GCCATAGTGA CGATGGTGGT |
| | | TTTGTCAAAA AGAAAAGGGG GGGATATGTA AGGAAAAGAG AGATCAGACT |
| | | TTCACTGTGT CTATGTAGAA AAGGAAGACA TAAGAAACTC CATTTTGATC |
| | | TGTACTAAGA AAAATTGTTT TGCCTTGAGA TGCTGTTAAT CTGTAACTTT |
| | | AGCCCCAACC CTGTGCTCAC GGAAACATGT GCTGTAAGGT TTAAGGGATC |
| | | TAGGGCTGTG CAGGATGTAC CTTGTTAACA ATATGTTTGC AGGCAGTATG |
| | | TTTGGTAAAA GTCATCGCCA TTCTCCATTC TCGATTAACC AGGGGCTCAA |
| | | TGCACTGTGG AAAGCCACAG GAACCTCTGC CCAAGAAAGC CTGGCTGTTG |
| | | TGGGAAGTCA GGGACCCCGA ATGGAGGGAC CAGCTGGTGC TGCATCAGGA |
| | | AACATAAATT GTGAAGATTT CTTGGACATT TATCAGTTTC CAAAATTAAT |
| | | ACTTTTATAA TTTCTTACAC CTGTCTTACT TTAATCTCTT AATCCTGTTA |
| | | TCTTTGTAAG CTGAGGATAT ACGTCACCTC AGGACCACTA TTGTACAAAT |
| | | TGATTGTAAA ACATGTTCAC ATGTGTTTGA ACAATATGAA ATCAGTGCAC |
| | | CTTGAAAATG AACAGAATAA CAGTGATTTT AGGGAACAAA GGAAGACAAC |
| | | CATAAGGTCT GACTGCCTGA GGGGTCGGGC AAAAAGCCAT ATTTTTCTTC |
| | | TTGCAGAGAG CCTATAAATG GACGTGCAAG TAGGAGAGAT ATTGCTAAAT |
| | | T |
| 6 | CAMKK2 | AGAGCAAGCT GAGCCGAGCC GAGCCGAGCT GGGGGCGCAG AGCGCGGGAG |
| | | GCGGCGGCGG CGCGGAGCCC AGGTGGCTCC GCTGCCGGAT GGGAGTGCCC |
| | | CAGTGTGCTG GATGAAGCTG GCGCATGCAC CATGTCATCA TGTGTCTCTA |
| | | GCCAGCCCAG CAGCAACCGG GCCGCCCCCC AGGATGAGCT GGGGGGCAGG |
| | | GGCAGCAGCA GCAGCGAAAG CCAGAAGCCC TGTGAGGCCC TGCGGGGCCT |
| | | CTCATCCTTG AGCATCCACC TGGGCATGGA GTCCTTCATT GTGGTCACCG |
| | | AGTGTGAGCC GGGCTGTGCT GTGGACCTCG GCTTGGCGCG GGACCGGCCC |
| | | CTGGAGGCCG ATGGCCAAGA GGTCCCCCTT GACACCTCCG GGTCCCAGGC |
| | | CCGGCCCCAC CTCTCCGGTC GCAAGCTGTC TCTGCAAGAG CGGTCCCAGG |
| | | GTGGGCTGGC AGCCGGTGGC AGCCTGGACA TGAACGGACG CTGCATCTGC |
| | | CCGTCCCTGC CCTACTCACC CGTCAGCTCC CCGCAGTCCT CGCCTCGGCT |
| | | GCCCCGGCGG CCGACAGTGG AGTCTCACCA CGTCTCCATC ACGGGTATGC |
| | | AGGACTGTGT GCAGCTGAAT CAGTATACCC TGAAGGATGA AATTGGAAAG |
| | | GGCTCCTATG GTGTCGTCAA GTTGGCCTAC AATGAAAATG ACAATACCTA |
| | | CTATGCAATG AAGGTGCTGT CCAAAAAGAA GCTGATCCGG CAGGCCGGCT |
| | | TTCCACGTCG CCCTCCACCC CGAGGCACCC GGCCAGCTCC TGGAGGCTGC |
| | | ATCCAGCCCA GGGGCCCCAT TGAGCAGGTG TACCAGGAAA TTGCCATCCT |
| | | CAAGAAGCTG GACCACCCCA ATGTGGTGAA GCTGGTGGAG GTCCTGGATG |
| | | ACCCCAATGA GGACCATCTG TACATGGTGT TCGAACTGGT CAACCAAGGG |
| | | CCCGTGATGG AAGTGCCCAC CCTCAAACCA CTCTCTGAAG ACCAGGCCCG |
| | | TTTCTACTTC CAGGATCTGA TCAAAGGCAT CGAGTACTTA CACTACCAGA |
| | | AGATCATCCA CCGTGACATC AAACCTTCCA ACCTCCTGGT CGGAGAAGAT |
| | | GGGCACATCA AGATCGCTGA CTTTGGTGTG AGCAATGAAT TCAAGGGCAG |
| | | TGACGCGCTC CTCTCCAACA CCGTGGGCAC GCCCGCCTTC ATGGCACCCG |
| | | AGTCGCTCTC TGAGACCCGC AAGATCTTCT CTGGGAAGGC CTTGGATGTT |
| | | TGGGCCATGG GTGTGACACT ATACTGCITT GTCTTTGGCC AGTGCCCATT |
| | | CATGGACGAG CGGATCATGT GTTTACACAG TAAGATCAAG AGTCAGGCCC |
| | | TGGAATTTCC AGACCAGCCC GACATAGCTG AGGACTTGAA GGACCTGATC |
| | | ACCCGTATGC TGGACAAGAA CCCCGAGTCG AGGATCGTGG TGCCGGAAAT |
| | | CAAGCTGCAC CCCTGGGTCA CGAGGCATGG GGCGGAGCCG TTGCCGTCGG |
| | | AGGATGAGAA CTGCACGCTG GTCGAAGTGA CTGAAGAGGA GGTCGAGAAC |
| | | TCAGTCAAAC ACATTCCCAG CTTGGCAACC GTGATCCTGG TGAAGACCAT |
| | | GATACGTAAA CGCTCCTTTG GGAACCCATT CGAGGGCAGC CGGCGGGAGG |
| | | AACGCTCACT GTCAGCGCCT GGAAACTTGC TCACCAAAAA ACCAACCAGG |
| | | GAATGTGAGT CCCTGTCTGA GCTCAAGGAA GCAAGGCAGC GAAGACAACC |
| | | TCCAGGGCAC CGACCCGCCC CCCGTGGGGG AGGAGGAAGT GCTCTTGTGA |
| | | GAGGCAGTCC CTGCGTGGAA AGTTGCTGGG CCCCCGCCCC CGGCTCCCCC |
| | | GCACGCATGC ATCCACTGCG GCCGGAGGAG GCCATGGAGC CCGAGTAGCT |
| | | GCCTGGATCG CTCGACCTCG CATGCGCGCC GCGTCGCCTC TGGGGGGCTG |
| | | CTGCACCGCG TTTCCATAGC AGCATGTCCT ACGGAAACCC AGCACGTGTG |
| | | TAGAGCCTCG ATCGTCATCT CTGGTTATTT GTTTTTTCCT TTGTTGTTTT |
| | | AAAGGGGACA AAAAAAAAAA AAGGACTTGA CTCCATGACG TCGACCGTGG |
| | | CCGCTGGCTG GCTGGACAGG CGGGTGTGAG GAGTTGCAGA CCCAAACCCA |
| | | CGTGCATTTT GGGACAATTG CTTTTTAAAA CGTTTTTATG CCAAAAATCC |
| | | TTCATTGTGA TTTTCAGAAC CACGTCAGAT ATACCAAGTG ACTGTGTGTG |
| | | GGGTTTGACA ACTGTGGAAA GGCGAGCAGA AAACTCCGGC GGTCTGAGGC |
| | | CATGGAGGTG GTTGCTGCAT TTGAGAGGGA GTAGGGGGCT AGATGTGGCT |
| | | CCTAGTGCAA ACCGGAAACC ATGGCACCTT CCAGAGCCGT GGTCTCAAGG |
| | | AGTCAGAGCA GGGCTGGCCC TCAGTAGCTG CAGGGAGCTT TGATGCAACT |
| | | TATTTGTAAG AAGGATTTTT AAATTTTTTA TGGGTAGAAT TGTAGTCAGG |
| | | AAAACAGAAA GGGCTTGAAA TTTAATAAGT GCTGCTGGAA GGGGATTTTC |
| | | CAAGCCTGGA AGGGTATTCA GCAGCTGIGG TGGGGAAACA TTTCTCCTGA |
| | | AAGACTGAAC GTGTTTCTTC ATGACAGCTG CTCAAAGCAG GTTTCTGAGA |
| | | TAGCTGACCG AGCTCTGGTA AATCTCTTTG TCAAATTACG AAAACTTCAG |
| | | GGTGAAATCC TATGCTTCCA TGTACATTAC ATGGCTTAAG ATTAAACAAA |
| | | AACATTTTTC AAGTCTCTAA CTAGAGTGAA CTCTAGAGCA CAGTAGTTCA |
| | | GAAACTATTT AGAGCTTCCA GGATATATTT CACAGCTTCA GGCATGTGAT |
| | | CAGTTAGAGC CGATGAAACC TATGCCCGCC TGTATATATA TTAGCAGCTT |
| | | AGCTAGTTCA TAACCTGTAT ATTCTAAAGA CTGCTAAGGT TTTGTTTTCA |
| | | TTTTAAATCC TAGCTGATTG TTGTGGTCAA TGAAATACCC AGTTTCTGGA |
| | | GGGCCAGGTG GGAAATGCTT TCACTGGACC AACACACAAA TGATCATCCT |
| | | GAGGATCTGA GCTTCCCTAG ACTCCACACA ATAACCTTGG GGCACCCTTT |
| | | TAGAGAAGAC TGTTGAAACC CACAGCACTC GTTGGGGTAT GAGGAAACCA |
| | | GGGCTTGGCA CAGGAAGTTC CCCTTTGTAG CTAAAAGTCC AGAAAGAAAG |
| | | GGTTCATCTT TTTGACTTCC AACTGATATT GGGAAGTTTG GTTGAGGTTC |
| | | AAGTGTGACT CCTTCCAGAG CCACAGGTAG GGGAGTGTGA AGTTGAGGGG |
| | | GAGGAAAGCT GGAAGGACTC TGCCTTGGGA GATTCCCAGC TCTGCTTTCC |
| | | AGCGCTTGGT GGAATCTGGG CTGGGGAAAG ACGGCACCGG GAAACTCTGC |
| | | TTCCCCATTG TTTCCATCTG ATCAGCTGTG GTGTGAGGAC TTCTCAGACA |
| | | AAGGCAAGGC CTCGTGCCCC TGCCCAGCCC ATTCATGGAG CCCTGGGCCT |
| | | TCTTGGCTTC CATAGATCCT AAGCTCTTGA CTGTAGTTTA GCCAGACTTG |
| | | TTTTGCTATC TTATAAGCAG TTCAGAATTA GGGAATGCTG GTTTTGAAGA |
| | | GCAAAGGACA GGTAGTCTAG AGAGGGTCGT CTGGCCTGCT TGCTGGGTCT |
| | | TTGTAACCCA GCACTTCCTC TTGCCCTCCT GGCTTTATGT TTATGGGGAG |
| | | AGGACTCAAT AGCTCCACCC CTTCTGGCAC CAGATGGGGC TTGGTTAGTT |
| | | TGCAATAAGC ACCTTGCAGA GGTTAAAGCC AGCGGGTCCC TAGTCTTAGG |
| | | CCCAGCCTGC TTGTGTGGGC TCTGGCCTGG CCTGGTGGCT GGCCCAGGGG |
| | | GCAGCAGTGC TTAGAGCTTC TGCAGGGCTT CTCTTGTTTA CACAGCTGCA |
| | | TCAGACAATG CCATTTCTCC CCACCACGGA ACCTTCCATC TAAGATTTCT |
| | | TCCAGGGAAT GCCAGCAATC AGGCAGCACC CAGCTGTGGG GGCAGTGGGG |
| | | TGGGGGAGAC CCACATTGAT GACTTTTTTT TTTTCTTTTA ATGAAGAAAC |
| | | ACCAAAGAAA GCTGTGGAAA GGACCTGCCC CACATGAAAA GGATAAGCCA |
| | | AGATGGCTGT AAACACAGAG CATTTGAGCT GCCACTCTTG GAGCACATTG |
| | | ATTTTTCAAA AGCCAGCTCT GTCAGGAAAG GAGGTGCTGT TATGAGCAGC |
| | | TCTTCCAGTG GGCAAAGAGG ACGCCCATAA TTTCTTCCAT TGCTAGCTCA |
| | | TCTGTGGGAC CAATTTGGTG TAAGCAACCT GTGGCCTGCA CTTGTGGCCT |
| | | CGAAGGAAGC ACAAACCCTC CATCCACTTC CCATTTCCTC TGCCCTTTTC |
| | | CACCTCCCCC TTCCATCCCA CCAGCTGCCA GTGGCTCCCA GAAAGCCTTA |
| | | TTGAGCCCCT TGTTGACACT TGGGGCTGCG GAGGCCTCTC CCTACTGGTC |
| | | TGGCCTTTCC TGAGAGGCAG GTCTTCCGTC CTCAGAGCCT TTCTGGAACA |
| | | AGGAGAATGC CTGTGCAGGT GGACACACAG GCCTGGCCTG TCGCTCTCAC |
| | | TTGTCTTCCA GCGGGGAGCT TCACGTTGCC GAGTGGAAGA ACCATGACCT |
| | | CCACTTGCTT CCAAGGTGCT AGGGAAGTTT CAGGGTACGC TGGTTCCCCT |
| | | CTCCAGCTGG AGGCCGAGTT TCTGGGGACT GCAGATTTTT CTACTCTGTG |
| | | ATCGATTCAA TGCCCGATGC TTCTGTTTCA TTCCCGACCC TTTCTACTAT |
| | | GCATTTTCCT TTTATCAGGT GTATAAAGTT AAATACTGTG TATTTATCAC |
| | | TAAAAAGTAC ATGAACTTAA GAGACAACTA AGCCTTTCGT GTTTTTCCAC |
| | | AGGTGTTTAA GCTTCTCTGT ACAGTTGAAA TAAACAGACA GCAAAATGGT |
| | | GCCAA |
| 7 | PCA3 | ACAGAAGAAA TAGCAAGTGC CGAGAAGCTG GCATCAGAAA AACAGAGGGG |
| | | AGATTTGTGT GGCTGCAGCC GAGGGAGACC AGGAAGATCT GCATGGTGGG |
| | | AAGGACCTGA TGATACAGAG GTGAGAAATA AGAAAGGCTG CTGACTTTAC |
| | | CATCTGAGGC CACACATCTG CTGAAATGGA GATAATTAAC ATCACTAGAA |
| | | ACAGCAAGAT GACAATATAA TGTCTAAGTA GTGACATGTT TTTGCACATT |
| | | TCCAGCCCCT TTAAATATCC ACACACACAG GAAGCACAAA AGGAAGCACA |
| | | GAGATCCCTG GGAGAAATGC CCGGCCGCCA TCTTGGGTCA TCGATGAGCC |
| | | TCGCCCTGTG CCTGGTCCCG CTTGTGAGGG AAGGACATTA GAAAATGAAT |
| | | TGATGTGTTC CTTAAAGGAT GGGCAGGAAA ACAGATCCTG TTGTGGATAT |
| | | TTATTTGAAC GGGATTACAG ATTTGAAATG AAGTCACAAA GTGAGCATTA |
| | | CCAATGAGAG GAAAACAGAC GAGAAAATCT TGATGGCTTC ACAAGACATG |
| | | CAACAAACAA AATGGAATAC TGTGATGACA TGAGGCAGCC AAGCTGGGGA |
| | | GGAGATAACC ACGGGGCAGA GGGTCAGGAT TCTGGCCCTG CTGCCTAAAC |
| | | TGTGCGTTCA TAACCAAATC ATTTCATATT TCTAACCCTC AAAACAAAGC |
| | | TGTTGTAATA TCTGATCTCT ACGGTTCCTT CTGGGCCCAA CATTCTCCAT |
| | | ATATCCAGCC ACACTCATTT TTAATATTTA GTTCCCAGAT CTGTACTGTG |
| | | ACCTTTCTAC ACTGTAGAAT AACATTACTC ATTTTGTTCA AAGACCCTTC |
| | | GTGTTGCTGC CTAATATGTA GCTGACTGTT TTTCCTAAGG AGTGTTCTGG |
| | | CCCAGGGGAT CTGTGAACAG GCTGGGAAGC ATCTCAAGAT CTTTCCAGGG |
| | | TTATACTTAC TAGCACACAG CATGATCATT ACGGAGTGAA TTATCTAATC |
| | | AACATCATCC TCAGTGTCTT TGCCCATACT GAAATTCATT TCCCACTTTT |
| | | GTGCCCATTC TCAAGACCTC AAAATGTCAT TCCATTAATA TCACAGGATT |
| | | AACTTTTTTT TTTAACCTGG AAGAATTCAA TGTTACATGC AGCTATGGGA |
| | | ATTTAATTAC ATATTTTGTT TTCCAGTGCA AAGATGACTA AGTCCTTTAT |
| | | CCCTCCCCTT TGTTTGATTT TTTTTCCAGT ATAAAGTTAA AATGCTTAGC |
| | | CTTGTACTGA GGCTGTATAC AGCCACAGCC TCTCCCCATC CCTCCAGCCT |
| | | TATCTGTCAT CACCATCAAC CCCTCCCATG CACCTAAACA AAATCTAACT |
| | | TGTAATTCCT TGAACATGTC AGGCATACAT TATTCCTTCT GCCTGAGAAG |
| | | CTCTTCCTTG TCTCTTAAAT CTAGAATGAT GTAAAGTTTT GAATAAGTTG |
| | | ACTATCTTAC TTCATGCAAA GAAGGGACAC ATATGAGATT CATCATCACA |
| | | TGAGACAGCA AATACTAAAA GTGTAATTTG ATTATAAGAG TTTAGATAAA |
| | | TATATGAAAT GCAAGAGCCA CAGAGGGAAT GTTTATGGGG CACGTTTGTA |
| | | AGCCTGGGAT GTGAAGCAAA GGCAGGGAAC CTCATAGTAT CTTATATAAT |
| | | ATACTTCATT TCTCTATCTC TATCACAATA TCCAACAAGC TTTTCACAGA |
| | | ATTCATGCAG TGCAAATCCC CAAAGGTAAC CTTTATCCAT TTCATGGTGA |
| | | GTGCGCTTTA GAATTTTGGC AAATCATACT GGTCACTTAT CTCAACTTTG |
| | | AGATGTGTTT GTCCTTGTAG TTAATTGAAA GAAATAGGGC ACTCTTGTGA |
| | | GCCACTTTAG GGTTCACTCC TGGCAATAAA GAATTTACAA AGAGCTACTC |
| | | AGGACCAGTT GTTAAGAGCT CTGTGTGTGT GTGTGTGTGT GTGAGTGTAC |
| | | ATGCCAAAGT GTGCCTCTCT CTCTTTGACC CATTATTICA GACTTAAAAA |
| | | CAAGCATGTT TTCAAATGGC ACTATGAGCT GCCAATGATG TATCACCACC |
| | | ATATCTCATT ATTCTCCAGT AAATGTGATA ATAATGTCAT CTGTTAACAT |
| | | AAAAAAAGTT TGACTTCACA AAAGCAGCTG GAAATGGACA ACCACAATAT |
| | | GCATAAATCT AACTCCTACC ATCAGCTACA CACTGCTTGA CATATATTGT |
| | | TAGAAGCACC TCGCATTTGT GGGTTCTCTT AAGCAAAATA CTTGCATTAG |
| | | GTCTCAGCTG GGGCTGTGCA TCAGGCGGTT TGAGAAATAT TCAATTCTCA |
| | | GCAGAAGCCA GAATTTGAAT TCCCTCATCT TTTAGGAATC ATTTACCAGG |
| | | TTTGGAGAGG ATTCAGACAG CTCAGGTGCT TTCACTAATG TCTCTGAACT |
| | | TCTGTCCCTC TTTGTGTTCA TGGATAGTCC AATAAATAAT GTTATCTTTG |
| | | AACTGATGCT CATAGGAGAG AATATAAGAA CTCTGAGTGA TATCAACATT |
| | | AGGGATTCAA AGAAATATTA GATTTAAGCT CACACTGGTC AAAAGGAACC |
| | | AAGATACAAA GAACTCTGAG CTGTCATCGT CCCCATCTCT GTGAGCCACA |
| | | ACCAACAGCA GGACCCAACG CATGTCTGAG ATCCTTAAAT CAAGGAAACC |
| | | AGTGTCATGA GTTGAATTCT CCTATTATGG ATGCTAGCTT CTGGCCATCT |
| | | CTGGCTCTCC TCTTGACACA TATTAGCTTC TAGCCTTTGC TTCCACGACT |
| | | TTTATCTTTT CTCCAACACA TCGCTTACCA ATCCTCTCTC TGCTCTGTTG |
| | | CTTTGGACTT CCCCACAAGA ATTTCAACGA CTCTCAAGTC TTTTCTTCCA |
| | | TCCCCACCAC TAACCTGAAT GCCTAGACCC TTATTTTTAT TAATTTCCAA |
| | | TAGATGCTGC CTATGGGCTA TATTGCTTTA GATGAACATT AGATATTTAA |
| | | AGCTCAAGAG GTTCAAAATC CAACTCATTA TCTTCTCTTT CTTTCACCTC |
| | | CCTGCTCCTC TCCCTATATT ACTGATTGCA CTGAACAGCA TGGTCCCCAA |
| | | TGTAGCCATG CAAATGAGAA ACCCAGTGGC TCCTTGTGGT ACATGCATGC |
| | | AAGACTGCTG AAGCCAGAAG GATGACTGAT TACGCCTCAT GGGTGGAGGG |
| | | GACCACTCCT GGGCCTTCGT GATTGTCAGG AGCAAGACCT GAGATGCTCC |
| | | CTGCCTTCAG TGTCCTCTGC ATCTCCCCTT TCTAATGAAG ATCCATAGAA |
| | | TTTGCTACAT TTGAGAATTC CAATTAGGAA CTCACATGTT TTATCTGCCC |
| | | TATCAATTTT TTAAACTTGC TGAAAATTAA GTTTTTTCAA AATCTGTCCT |
| | | TGTAAATTAC TTTTTCTTAC AGTGTCTTGG CATACTATAT CAACTTTGAT |
| | | TCTTTGTTAC AACTTTTCTT ACTCTTTTAT CACCAAAGTG GCTTTTATTC |
| | | TCTTTATTAT TATTATTTTC TTTTACTACT ATATTACGTT GTTATTATTT |
| | | TGTTCTCTAT AGTATCAATT TATTTGATTT AGTTTCAATT TATTTTTATT |
| | | GCTGACTTTT AAAATAAGTG ATTCGGGGGG TGGGAGAACA GGGGAGGGAG |
| | | AGCATTAGGA CAAATACCTA ATGCATGTGG GACTTAAAAC CTAGATGATG |
| | | GGTTGATAGG TGCAGCAAAC CACTATGGCA CACGTATACC TGTGTAACAA |
| | | ACCTACACAT TCTGCACATG TATCCCAGAA CGTAAAGTAA AATTTAAAAA |
| | | AAAGTGA |
| 8 | NKAIN1 | AGTGCTGCTC TGCGCTGCGC CGCGCTCGGG GCTCGCTCTC CTTGCTCCGC |
| | | GCTCCCCGCC AGCCGCCCCG GGGCAGGAGG CGCGCCTGAC GGACGGCCCG |
| | | CTAGACAAAG GAGGCGCGGC TCGGCGGGGC CAGCGCGCGG ACGGACGGAC |
| | | CATGGACTCG GAGCGCGGGC GGCCGGCCCC AGCCTTGGGG ACCGGACACT |
| | | CCCGGGCCCG GCCCTAGGCG CCCGGCCCCG CCGCCCGGCG CGCCCAGCGG |
| | | GGAGGACGTG GAGCCCGCGC GGCGCGAGCA GGCGGCGGCC GCGGAGCAAG |
| | | AAGGGCGCCG CGGCGTGCGG CCCGCGCAGC CCCCGGAGCC ATGGGCAAGT |
| | | GCAGCGGGCG CTGCACGCTG GTCGCCTTCT GCTGCCTGCA GCTGGTGGCT |
| | | GCGCTGGAGC GGCAGATCTT TGACTTCCTG GGCTACCAGT GGGCTCCCAT |
| | | CCTAGCCAAC TTCCTGCACA TCATGGCAGT CATCCTGGGC ATCTTTGGCA |
| | | CCGTGCAGTA CCGCTCCCGG TACCTCATCC TGTATGCAGC CTGGCTGGTG |
| | | CTCTGGGTTG GCTGGAATGC ATTTATCATC TGCTTCTACT TGGAGGTTGG |
| | | ACAGCTGTCC CAGGACCGGG ACTTCATCAT GACCTTCAAC ACATCCCTGC |
| | | ACCGCTCCTG GTGGATGGAG AATGGGCCAG GCTGCCTGGT GACACCTGTT |
| | | CTGAACTCCC GCCTGGCTCT GGAGGACCAC CATGTCATCT CTGTCACTGG |
| | | CTGCCTGCTT GACTACCCCT ACATTGAAGC CCTCAGCAGC GCCCTGCAGA |
| | | TCTTCCTGGC ACTGTTCGGC TTCGTGTTCG CCTGCTACGT GAGCAAAGTG |
| | | TTCCTGGAGG AGGAGGACAG CTTTGACTTC ATCGGCGGCT TTGACTCCTA |
| | | CGGATACCAG GCGCCCCAGA AGACGTCGCA TTTACAGCTG CAGCCTCTGT |
| | | ACACGTCGGG GTAGCCTCTG CCCCGCGCCC ACCCCGGCGC CTCGCCCTGG |
| | | GCTGACCGCA GCTGCCGCGA GCTCGGGCCA AGGCGCAGGC GTGTCCCCCT |
| | | GGTGGCCCGC GCGCTCACTG CAGCCTGTGC CCAACCCCGC GTCTGCATCT |
| | | GGAGATGCGG ACTTGGACGT GGACTTGGAC TTGGACTTGG ATTTGAGCTT |
| | | GGCTCTTCGC AGCCCGGACT TCGGAGGAGT GGGGCGGGGC GGGGGAGGGG |
| | | CACCACGGGT TTTTTGTTTT TTGTTTGITT GTTTTTAATC TCAGCCTTGG |
| | | CGTGAGCTGG GGCCTTCCTC TCTTCTCCAG CCTCTCCCTT TCACTCTTCA |
| | | CCCAGCATCC TGCCCCCCTG TCCAAAAACA GCAGGACATC AGACCCATCC |
| | | CATCCCACCA CACTCACTCA CCAGCTCTGG GGAAAGCTAC TGTGAACTAG |
| | | GAGCAGGATT CCTGGGTTCT AATCGCAGGT CCATCACTGA CTGTGACGTC |
| | | TAGCAAAGCC CTTGCCCTCT CTGAGCCTCG GTTTCCGCAC CTCAAGTAAT |
| | | TAATCCCTTA GCAAATGGAC TCTTTTAGAC TTCTCATTTA ACTCAATTCC |
| | | CTGAGCTAGA CTGGGATTAA AATTCTCATT TTGCAGTACA TTAAAACTGA |
| | | GGCCCAGAGA TGTGATTTGC TTGAGGCCAC ACAGCTAGAT TTTTGGTGGA |
| | | AGTGGGCCTT GAACACAGTG TACTTTCTGC AGTTTCTGAC TGTAAAACCC |
| | | AGTGTCTGCT CTCTGAGTTC CATTTCCAAG CCCCCCTCCA TCTTGGACCT |
| | | ATGTGGTCTC CACCATATTC ACACACCACC ACCACCACTT GCCAATGCCT |
| | | CTCTTAAAGC AATATACCCA TTCGTTCTCT TATTGGGAAC TGGATGGATG |
| | | AAGCCCCAAA TTCAGCCCCA CCCACAGAGA AGCCTTCCTA CACTCAGCCT |
| | | CTGTCCACCC TTGGCAAATC TTTCAAGCTC TCTCCTCCAG GAAAGTGGGG |
| | | CCCCAACTCA GTCACTCCAC CCCCTTCCAG GTCCCTGAGG CTGGTTCTAC |
| | | TGTATCCCCA TCACCTCCAC AACTCCACTC ACCCCTGACG GCTCCATCCA |
| | | CCTCACCAGT TGGAAGGCTT GTGGTTTCAG AGAGGAGCAA TGCTGGTCAG |
| | | CGCTGCCCAG ACTCCAGTGT TTACAGATCA CCAGCATTTA CAACCAATCC |
| | | AATGGCCAGA AGCCTCCTCT AACAAGCCCA GAAGGAGTTC TGAAGGGGCA |
| | | GATGGGGGTG TGAGTAGTCG GGGAGTCGGG ATTGCCAGCA CCCTCACCCT |
| | | TCCTTGGGGG CAAGTAGAGG TGAGAACACT TTCCCCACCT CCCTCCACAG |
| | | ACACTCCTGA GGACGCTGCA TCCCACGCAC TGCCTGGTGC GTCCATAGAG |
| | | AGAGGATCAG GTCTCAGCAT TTCATCTGTG AAAGAGGCAT GGCCCTGGGT |
| | | TAGAAAGGAG GGCAGGAGAC ATGGAGGAAC TGGGGGGCAC CCAGATGGTG |
| | | CAGATGGTTT GCACACCTGA GCCTGTCTGT GGTGACCATT CCGCTCCTCT |
| | | CCCACTACCC TCCAATCTAT CATTCCCTAC TCTCTAAGGC CAAAATATCC |
| | | TGAGCAAGGC TGGCAACCCC ACCCCACCAT CCCAAATGCA AGCAGCCAGG |
| | | CCCAGGAGTT CCTCTGGCCC CCACAGGCAT GGAGCTCCCA GCTGGTGGGT |
| | | ACAGCTTGAG AGGGGGGCAG CTCCCTCAGG CTAAGCTACT GCCCTTCACT |
| | | GGGCCAGCCC TGCCTCCAGC CCTCACCTCT CTCACCCCAA CTCTCCCCCA |
| | | AGCCCCTTTC TACTCAACGG GTGTAGCCAC TGGTGCTTTG AAGCCTTTTG |
| | | TTTTTATAAG ATGGTTTTTG CAAGGGGACC AGGTTCTCTT TTCACTGGGA |
| | | CCTTGCAAGG AGGGGAGTGC TCTCCTGGTT TCTGTGCAGG CGGGTTGATT |
| | | AAAGATGGTG TTTTCTTCTC TA |
| 9 | B3GNT6 | AGTGTGTGAA GTAAAGGGAT TAAAGGCTAG TCTCAGGCTG GGGATGGCTC |
| | | CTGTCTATTT CTTCTCTCTC AGAGACTGCA GATGGCTTTT CCCTGCCGCA |
| | | GGTCCCTGAC TGCCAAGACT CTGGCCTGCC TCCTGGTGGG CGTGAGTTTC |
| | | TTAGCACTGC AGCAGTGGTT CCTCCAGGCG CCAAGGTCCC CGCGGGAGGA |
| | | GAGGTCCCCG CAGGAGGAGA CGCCAGAGGG TCCCACCGAC GCTCCCGCGG |
| | | CTGACGAGCC GCCCTCGGAG CTCGTCCCCG GGCCCCCGTG CGTGGCGAAC |
| | | GCCTCGGCGA ACGCCACGGC CGACTTCGAG CAGCTGCCCG CGCGCATCCA |
| | | GGACTTCCTG CGGTACCGCC ACTGCCGCCA CTTCCCGCTG CTTTGGGACG |
| | | CACCGGCCAA GTGCGCCGGC GGCCGAGGCG TGTTCCTGCT CCTGGCGGTG |
| | | AAGTCGGCGC CTGAGCACTA CGAGCGACGC GAGCTCATCC GGCGCACGTG |
| | | GGGGCAAGAG CGCAGCTACG GCGGGCGGCC AGTGCGCCGC CTCTTTCTAT |
| | | TGGGCACCCC GGGCCCCGAG GACGAGGCGC GCGCGGAGCG GCTGGCGGAG |
| | | CTGGTGGCGC TGGAGGCGCG CGAGCACGGC GACGTGCTGC AGTGGGCCTT |
| | | CGCGGACACC TTCCTCAACC TCACGCTCAA GCACCTGCAC TTGCTCGACT |
| | | GGCTGGCTGC ACGCTGCCCG CACGCGCGCT TTCTGCTCAG CGGCGACGAC |
| | | GACGTGTTCG TGCACACCGC CAACGTAGTC CGCTTCCTGC AGGCGCAGCC |
| | | ACCCGGCCGC CACCTGTTCT CCGGCCAGCT CATGGAGGGC TCCGTGCCCA |
| | | TCCGCGACAG CTGGAGCAAG TACTTCGTGC CGCCGCAGCT CTTCCCCGGG |
| | | TCCGCTTACC CGGTGTACTG CAGCGGCGGC GGCTTCCTCC TGTCCGGCCC |
| | | CACGGCCCGG GCCCTGCGCG CGGCCGCCCG CCACACCCCG CTCTTCCCCA |
| | | TCGACGACGC CTACATGGGC ATGTGTCTGG AGCGCGCCGG CCTGGCGCCC |
| | | AGCGGCCACG AGGGCATCCG ACCCTTCGGC GTGCAGCTGC CTGGCGCACA |
| | | GCAGTCCTCC TTCGACCCCT GCATGTACCG CGAGTTGCTG CTAGTGCACC |
| | | GCTTCGCGCC CTACGAGATG CTGCTCATGT GGAAGGCGCT GCACAGCCCC |
| | | GCGCTCAGCT GTGACCGGGG ACACCGGGTC TCCTGAGGCC AGTTGGGCGG |
| | | CTTCAGCCCC GGGCCTCCAA CCATGTCCAT GCTGAGAAGG CAGCTTTCCC |
| | | GCTCTGGGTA CCTTACGTCC TGCCCAGCTC TGTGCACCTG AACCCCAGCT |
| | | GCGCACTGAA ATCAGCTGGG GTGGGGGGTG TGGAAAATGC CTACATCCTG |
| | | GCTCCATCTC CCGAAGTTTC GATTTGATTA GTCTGGGGTG GACCCAGACA |
| | | TGTTAAGTAT TTTTTAAGTT CCTCCAGTGA TGCGAATGTG CAGCTAGGCC |
| | | TGAGGACCAC TCGGCTAGAC TATCTCTTCA TCCTCGCAAA GCCAGCTCCA |
| | | CCGCCCTCTC TGCAAGAATT CCGGGCCCCT CGCTCCCACA CTCGGGTCCT |
| | | CTTGAGCAGT GGAGCAAGGG AGACCTGGGA GCGTGGGAGC CAGGATCAGC |
| | | GCCCCCTGCC ATGTGCCTAC AAATGTCAGT TGTGATTTCC ACTGTTTACA |
| | | AGTGAGTGGA GCTGGAGCTG GGCTGACAGT ATCAGGTGGA TCCCGCTTCC |
| | | CCCTCCCCCA AGAAGTCAGC CAACACGCAG CTGAGGCGCA TGTGGTGGCC |
| | | TTCTTCCCAC CACTACCCCA GTACACCGTG AGGTAGAAAT CTTCACCGTG |
| | | CAAAGTGGAA ACCAGAGGCC CGGTCAGACA GTGACTAATC CAGGGCCGTG |
| | | GCATTCCCAG ACAGCACACC ACTGTGGTCC CCTCCACACT CACCCCAACC |
| | | AAAGCTAATG GCCTAGTTGG GTCCTGCCCG CCAATAATCA CCCCCACGGG |
| | | TCAGAGACAG GCTCCTTGCC GGGGTCTGGG CCTCAGGCTC AGTGGGCCTT |
| | | GGACAACCCA GCAGGGAGTT CCGGGGAGTC CGAAGTGGAG AAAGGCTGGT |
| | | GGGAACATGG AGGCCAGTGT TGGGGAGCCT GTGGAGGCAG GTGTGTAGAA |
| | | TTGTGTTCGG GAGGTGGGGG ATCTGAGACC GAAGTGGACA GTGGTTAAGA |
| | | TTGTGGGGCC GGGCGAGGTG GCTCACGCCT GTAATCCCAG CACTTTGGGA |
| | | GGCTGAGGAG GTCGGATCAT GAGGTCAAGA GTTCGAGACC AGCCTGGCCA |
| | | ATATGGTGAA ACCCCGTCTC TATTGGGAGT ACAAAAATTA GCCGGCCATA |
| | | GTGGCTCGTG CCTGTAATCT CAGCTATTTG GGAGGCTGAG GCAGGAGAAT |
| | | CACTTGAACC TGGGAGGCGG AGGTTGCAGT GAGCCGAGAT CGTGCCACTG |
| | | CACTCCAGCC TGGGCGACAG AGCAAGACTG CATCTCAAAA AAAAAAAAAA |
| | | AAA |
| 10 | TFF3 | GAGTCCTGAG CTGCGTCCCG GAGCCCACGG TGGTCATGGC TGCCAGAGCG |
| | | CTCTGCATGC TGGGGCTGGT CCTGGCCTTG CTGTCCTCCA GCTCTGCTGA |
| | | GGAGTACGTG GGCCTGTCTG CAAACCAGTG TGCCGTGCCA GCCAAGGACA |
| | | GGGTGGACTG CGGCTACCCC CATGTCACCC CCAAGGAGTG CAACAACCGG |
| | | GGCTGCTGCT TTGACTCCAG GATCCCTGGA GTGCCTTGGT GTTTCAAGCC |
| | | CCTGCAGGAA GCAGAATGCA CCTTCTGAGG CACCTCCAGC TGCCCCCGGC |
| | | CGGGGGATGC GAGGCTCGGA GCACCCTTGC CCGGCTGTGA TTGCTGCCAG |
| | | GCACTGTTCA TCTCAGCTTT TCTGTCCCTT TGCTCCCGGC AAGCGCTTCT |
| | | GCTGAAAGTT CATATCTGGA GCCTGATGTC TTAACGAATA AAGGTCCCAT |
| | | GCTCCACCCG AGGACAGTTC TTCGTGCCTG AGACTTTCTG AGGTTGTGCT |
| | | TTATTTCTGC TGCGTCGTGG GAGAGGGCGG GAGGGTGTCA GGGGAGAGTC |
| | | TGCCCAGGCC TCAAGGGCAG GAAAAGACTC CCTAAGGAGC TGCAGTGCAT |
| | | GCAAGGATAT TITGAATCCA GACTGGCACC CACGTCACAG GAAAGCCTAG |
| | | GAACACTGTA AGTGCCGCTT CCTCGGGAAA GCAGAAAAAA TACATTTCAG |
| | | GTAGAAGTTT TCAAAAATCA CAAGTCTTTC TTGGTGAAGA CAGCAAGCCA |
| | | ATAAAACTGT CTTCCAAAGT GGTCCTTTAT TTCACAACCA CTCTCGCTAC |
| | | TGTTCAATAC TTGTACTATT CCTGGGTTTT GTTTCTTTGT ACAGTAAACA |
| | | TTATGAACAA ACAGGCA |
| 11 | SPON2 | ACCCGACCGC TGCCGGCCGC GCTCCCGCTG CTCCTGCCGG GTGATGGAAA |
| | | ACCCCAGCCC GGCCGCCGCC CTGGGCAAGG CCCTCTGCGC TCTCCTCCTG |
| | | GCCACTCTCG GCGCCGCCGG CCAGCCTCTT GGGGGAGAGT CCATCTGTTC |
| | | CGCCAGAGCC CTGGCCAAAT ACAGCATCAC CTTCACGGGC AAGTGGAGCC |
| | | AGACGGCCTT CCCCAAGCAG TACCCCCTGT TCCGCCCCCC TGCGCAGTGG |
| | | TCTTCGCTGC TGGGGGCCGC GCATAGCTCC GACTACAGCA TGTGGAGGAA |
| | | GAACCAGTAC GTCAGTAACG GGCTGCGCGA CTTTGCGGAG CGCGGCGAGG |
| | | CCTGGGCGCT GATGAAGGAG ATCGAGGCGG CGGGGGAGGC GCTGCAGAGC |
| | | GTGCACGCGG TGTTTTCGGC GCCCGCCGTC CCCAGCGGCA CCGGGCAGAC |
| | | GTCGGCGGAG CTGGAGGTGC AGCGCAGGCA CTCGCTGGTC TCGTTTGTGG |
| | | TGCGCATCGT GCCCAGCCCC GACTGGTTCG TGGGCGTGGA CAGCCTGGAC |
| | | CTGTGCGACG GGGACCGTTG GCGGGAACAG GCGGCGCTGG ACCTGTACCC |
| | | CTACGACGCC GGGACGGACA GCGGCTTCAC CTTCTCCTCC CCCAACTTCG |
| | | CCACCATCCC GCAGGACACG GTGACCGAGA TAACGTCCTC CTCTCCCAGC |
| | | CACCCGGCCA ACTCCTTCTA CTACCCGCGG CTGAAGGCCC TGCCTCCCAT |
| | | CGCCAGGGTG ACACTGGTGC GGCTGCGACA GAGCCCCAGG GCCTTCATCC |
| | | CTCCCGCCCC AGTCCTGCCC AGCAGGGACA ATGAGATTGT AGACAGCGCC |
| | | TCAGTTCCAG AAACGCCGCT GGACTGCGAG GTCTCCCTGT GGTCGTCCTG |
| | | GGGACTGTGC GGAGGCCACT GTGGGAGGCT CGGGACCAAG AGCAGGACTC |
| | | GCTACGTCCG GGTCCAGCCC GCCAACAACG GGAGCCCCTG CCCCGAGCTC |
| | | GAAGAAGAGG CTGAGTGCGT CCCTGATAAC TGCGTCTAAG ACCAGAGCCC |
| | | CGCAGCCCCT GGGGCCCCCC GGAGCCATGG GGTGTCGGGG GCTCCTGTGC |
| | | AGGCTCATGC TGCAGGCGGC CGAGGGCACA GGGGGTTTCG CGCTGCTCCT |
| | | GACCGCGGTG AGGCCGCGCC GACCATCTCT GCACTGAAGG GCCCTCTGGT |
| | | GGCCGGCACG GGCATTGGGA AACAGCCTCC TCCTTTCCCA ACCTTGCTTC |
| | | TTAGGGGCCC CCGTGTCCCG TCTGCTCTCA GCCTCCTCCT CCTGCAGGAT |
| | | AAAGTCATCC CCAAGGCTCC AGCTACTCTA AATTATGTCT CCTTATAAGT |
| | | TATTGCTGCT CCAGGAGATT GTCCTTCATC GTCCAGGGGC CTGGCTCCCA |
| | | CGTGGTTGCA GATACCTCAG ACCTGGTGCT CTAGGCTGTG CTGAGCCCAC |
| | | TCTCCCGAGG GCGCATCCAA GCGGGGGCCA CTTGAGAAGT GAATAAATGG |
| | | GGCGGTTTCG GAAGCGTCAG TGTTTCCATG TTATGGATCT CTCTGCGTTT |
| | | GAATAAAGAC TATCTCTGTT GCTCACAAA |
| 12 | PCGEM1 | AAGGCACTCT GGCACCCAGT TTTGGAACTG CAGTTTTAAA AGTCATAAAT |
| | | TGAATGAAAA TGATAGCAAA GGTGGAGGTT TTTAAAGAGC TATTTATAGG |
| | | TCCCTGGACA GCATCTTTTT TCAATTAGGC AGCAACCTTT TTGCCCTATG |
| | | CCGTAACCTG TGTCTGCAAC TTCCTCTAAT TGGGAAATAG TTAAGCAGAT |
| | | TCATAGAGCT GAATGATAAA ATTGTACTAC GAGATGCACT GGGACTCAAC |
| | | GTGACCTTAT CAAGTGAGCA GGCTTGGTGC ATTTGACACT TCATGATATC |
| | | AGCCAAAGTG GAACTAAAAA CAGCTCCTGG AAGAGGACTA TGACATCATC |
| | | AGGTTGGGAG TCTCCAGGGA CAGCGGACCC TTTGGAAAAG GACTAGAAAG |
| | | TGTGAAATCT ATTAGTCTTC GATATGAAAT TCTCTGTCTC TGTAAAAGCA |
| | | TTTCATATTT ACAAGACACA GGCCTACTCC TAGGGCAGCA AAAAGTGGCA |
| | | ACAGGCAAGC AGAGGGAAAA GAGATCATGA GGCATTTCAG AGTGCACTGT |
| | | CTTTTCATAT ATTTCTCAAT GCCGTATGTT TGGTTTTATT TTGGCCAAGC |
| | | ATAACAATCT GCTCAAGAAA AAAAAATCTG GAGAAAACAA AGGTGCCTTT |
| | | GCCAATGTTA TGTTTCTTTT TGACAAGCCC TGAGATTTCT GAGGGGAATT |
| | | CACATAAATG GGATCAGGTC ATTCATTTAC GTTGTGTGCA AATATGATTT |
| | | AAAGATACAA CCTTTGCAGA GAGCATGCTT TCCTAAGGGT AGGCACGTGG |
| | | AGGACTAAGG GTAAAGCATT CTTCAAGATC AGTTAATCAA GAAAGGTGCT |
| | | CTTTGCATTC TGAAATGCCC TTGTTGCAAA TATTGGTTAT ATTGATTAAA |
| | | TTTACACTTA ATGGAAACAA CCTTTAACTT ACAGATGAAC AAACCCACAA |
| | | AAGCAAAAAA TCAAAAGCCC TACCTATGAT TTCATATTTT CTGTGTAACT |
| | | GGATTAAAGG ATTCCTGCTT GCTTTTGGGC ATAAATGATA ATGGAATATT |
| | | TCCAGGTATT GTTTAAAATG AGGGCCCATC TACAAATTCT TAGCAATACT |
| | | TTGGATAATT CTAAAATTCA GCTGGACATT GTCTAATTGT TTTTTATATA |
| | | CATCTTTGCT AGAATTTCAA ATTTTAAGTA TGTGAATTTA GTTAATTAGC |
| | | TGTGCTGATC AATTCAAAAA CATTACTTTC CTAAATTTTA GACTATGAAG |
| | | GTCATAAATT CAACAAATAT ATCTACACAT ACAATTATAG ATTGTTTTTC |
| | | ATTATAATGT CTTCATCTTA ACAGAATTGT CTTTGTGATT GTTTTTAGAA |
| | | AACTGAGAGT TTTAATTCAT AATTACTTGA TCAAAAAATT GTGGGAACAA |
| | | TCCAGCATTA ATTGTATGTG ATTGTTTTTA TGTACATAAG GAGTCTTAAG |
| | | CTTGGTGCCT TGAAGTCTTT TGTACTTAGT CCCATGTTTA AAATTACTAC |
| | | TTTATATCTA AAGCATTTAT GTTTTTCAAT TCAATTTACA TGATGCTAAT |
| | | TATGGCAATT ATAACAAATA TTAAAGATTT CGAAATAGAA AAAAAAAAAA |
| | | AAA |
| 13 | TRGV9 | GTGAGGACAC CGCTTTACAA CGATGCAGGG GGCCCCATGT CACCCTCACC |
| | | CATGGGAAGT TTGACTTGGT GGACTCAGCC AAGCCACAGA GGTCTAACGC |
| | | TTCTCTGCGG TGATTTCAGG CTGCCCTGGC AGAAAGCACA GTGCCTGCAG |
| | | ACATGCTGTC ACTGCTCCAC GCATCAACGC TGGCAGTCCT TGGGGCTCTG |
| | | TGTGTATATG GTGCAGGTCA CCTAGAGCAA CCTCAAATTT CCAGTACTAA |
| | | AACGCTGTCA AAAACAGCCC GCCTGGAATG TGTGGTGTCT GGAATAACAA |
| | | TTTCTGCAAC ATCTGTATAT TGGTATCGAG AGAGACCTGG TGAAGTCATA |
| | | CAGTTCCTGG TGTCCATTTC ATATGACGGC ACTGTCAGAA AGGAATCCGG |
| | | CATTCCGTCA GGCAAATTTG AGGTGGATAG GATACCTGAA ACGTCTACAT |
| | | CCACTCTCAC CATTCACAAT GTAGAGAAAC AGGACATAGC TACCTACTAC |
| | | TGTGCCTTGT TGGAGGGAAA TTATAAGAAA CTCTTTGGCA GTGGAACAAC |
| | | ACTTGTTGTC ACAGATAAAC AACTTGATGC AGATGTTTCC CCCAAGCCCA |
| | | CTATTTTTCT TCCTTCAATT GCTGAAACAA AGCTCCAGAA GGCTGGAACA |
| | | TACCTTTGTC TTCTTGAGAA ATTTTTCCCT GATGTTATTA AGATACATTG |
| | | GCAAGAAAAG AAGAGCAACA CGATTCTGGG ATCCCAGGAG GGGAACACCA |
| | | TGAAGACTAA CGACACATAC ATGAAATTTA GCTGGTTAAC GGTGCCAGAA |
| | | AAGTCACTGG ACAAAGAACA CAGATGTATC GTCAGACATG AGAATAATAA |
| | | AAACGGAGTT GATCAAGAAA TTATCTTTCC TCCAATAAAG ACAGATGTCA |
| | | TCACAATGGA TCCCAAAGAC AATTGTTCAA AAGATGCAAA TGATACACTA |
| | | CTGCTGCAGC TCACAAACAC CTCTGCATAT TACATGTACC TCCTCCTGCT |
| | | CCTCAAGAGT GTGGTCTATT TTGCCATCAT CACCTGCTGT CTGCTTAGAA |
| | | GAACGGCTTT CTGCTGCAAT GGAGAGAAAT CATAACAGAC GGTGGCACAA |
| | | GGAGGCCATC TTTTCCTCAT CGGTTATTGT CCCTAGAAGC GTCTTCTGAG |
| | | GATCTAGTTG GGCTTTCTTT CTGGGTTTGG GCCATTTCAG TTCTCATGTG |
| | | TGTACTATTC TATCATTATT GTATAACGGT TTTCAAACCA GTGGGCACAC |
| | | AGAGAACCTC ACTCTGTAAT AACAATGAGG AATAGCCACG GCGATCTCCA |
| | | GCACCAATCT CTCCATGTTT TCCACAGCTC CTCCAGCCAA CCCAAATAGC |
| | | GCCTGCTATA GTGTAGACAT CCTGCGGCTT CTAGCCTTGT CCCTCTCTTA |
| | | GTGTTCTTTA ATCAGATAAC TGCCTGGAAG CCTTTCATTT TACACGCCCT |
| | | GAAGCAGTCT TCTTTGCTAG TTGAATTATG TGGTGTGTTT TTCCGTAATA |
| | | AGCAAAATAA ATTTAAAAAA ATGAAAAGTT |
| 14 | TMSB15A | AACGCTAACC TGGTCCGGAG CGAGTCTGGG TCTCAGCCCC GCGAACAGCC |
| | | TTTCACGAGT CTTCAAGCTT TCAGGCTATC TTCTAGTCAA GATGAGTGAT |
| | | AAGCCAGACT TGTCGGAAGT GGAGAAGTTT GACAGGTCAA AACTGAAGAA |
| | | AACTAATACT GAAGAAAAAA ATACTCTTCC CTCAAAGGAA ACTATCCAGC |
| | | AAGAGAAAGA GTGTGTTCAA ACATCATAAA ATGGGGATCG CCTCCCAACA |
| | | GCAGATTTCG ACATTACCTG AGAGTCTTGA TTTTAGGCTT GTTTTTTGTA |
| | | AACCCATGTG TTTGTAGAGA TTTTAGGCGT CTTCGGATAT CTTCTCACCT |
| | | ATGTTCCCTG GCTAAGAAGT CAGAGGTAGC CAATGTTTCC TTAAATTCAT |
| | | TTTTAAACTT ACCATTGGTG CATATGTTCC AGATGGCAGA TGCTGTCAAT |
| | | AATCTCACCA TTGATGACCT TTGTGTATGT AGTTCTTGCA TCCTATACTG |
| | | GATAAGCCTG TTTTAACCTG CTATGATGGG TGCTTCCATT GCTTCATAAT |
| | | CTTCATGAAG TTGCATGCTT TTGCAGCTTT TCACAGTTTA TTTGCATTTC |
| | | TAATGTAGTA ATAAAGTAAC CAATATAATC ATTA |
| 15 | ERG | ATCCGCTCTA AACAACCTCA TCAAAACTAC TTTCTGGTCA GAGAGAAGCA |
| | | ATAATTATTA TTAACATTTA TTAACGATCA ATAAACTTGA TCGCATTATG |
| | | GCCAGCACTA TTAAGGAAGC CTTATCAGTT GTGAGTGAGG ACCAGTCGTT |
| | | GTTTGAGTGT GCCTACGGAA CGCCACACCT GGCTAAGACA GAGATGACCG |
| | | CGTCCTCCTC CAGCGACTAT GGACAGACTT CCAAGATGAG CCCACGCGTC |
| | | CCTCAGCAGG ATTGGCTGTC TCAACCCCCA GCCAGGGTCA CCATCAAAAT |
| | | GGAATGTAAC CCTAGCCAGG TGAATGGCTC AAGGAACTCT CCTGATGAAT |
| | | GCAGTGTGGC CAAAGGCGGG AAGATGGTGG GCAGCCCAGA CACCGTTGGG |
| | | ATGAACTACG GCAGCTACAT GGAGGAGAAG CACATGCCAC CCCCAAACAT |
| | | GACCACGAAC GAGCGCAGAG TTATCGTGCC AGCAGATCCT ACGCTATGGA |
| | | GTACAGACCA TGTGCGGCAG TGGCTGGAGT GGGCGGTGAA AGAATATGGC |
| | | CTTCCAGACG TCAACATCTT GTTATTCCAG AACATCGATG GGAAGGAACT |
| | | GTGCAAGATG ACCAAGGACG ACTTCCAGAG GCTCACCCCC AGCTACAACG |
| | | CCGACATCCT TCTCTCACAT CTCCACTACC TCAGAGAGAC TCCTCTTCCA |
| | | CATTTGACTT CAGATGATGT TGATAAAGCC TTACAAAACT CTCCACGGTT |
| | | AATGCATGCT AGAAACACAG GGGGTGCAGC TTTTATTTTC CCAAATACTT |
| | | CAGTATATCC TGAAGCTACG CAAAGAATTA CAACTAGGCC AGATTTACCA |
| | | TATGAGCCCC CCAGGAGATC AGCCTGGACC GGTCACGGCC ACCCCACGCC |
| | | CCAGTCGAAA GCTGCTCAAC CATCTCCTTC CACAGTGCCC AAAACTGAAG |
| | | ACCAGCGTCC TCAGTTAGAT CCTTATCAGA TTCTTGGACC AACAAGTAGC |
| | | CGCCTTGCAA ATCCAGGCAG TGGCCAGATC CAGCTTTGGC AGTTCCTCCT |
| | | GGAGCTCCTG TCGGACAGCT CCAACTCCAG CTGCATCACC TGGGAAGGCA |
| | | CCAACGGGGA GTTCAAGATG ACGGATCCCG ACGAGGTGGC CCGGCGCTGG |
| | | GGAGAGCGGA AGAGCAAACC CAACATGAAC TACGATAAGC TCAGCCGCGC |
| | | CCTCCGTTAC TACTATGACA AGAACATCAT GACCAAGGTC CATGGGAAGC |
| | | GCTACGCCTA CAAGTTCGAC TTCCACGGGA TCGCCCAGGC CCTCCAGCCC |
| | | CACCCCCCGG AGTCATCTCT GTACAAGTAC CCCTCAGACC TCCCGTACAT |
| | | GGGCTCCTAT CACGCCCACC CACAGAAGAT GAACTTTGTG GCGCCCCACC |
| | | CTCCAGCCCT CCCCGTGACA TCTTCCAGTT TTTTTGCTGC CCCAAACCCA |
| | | TACTGGAATT CACCAACTGG GGGTATATAC CCCAACACTA GGCTCCCCAC |
| | | CAGCCATATG CCTTCTCATC TGGGCACTTA CTACTAAAGA CCTGGCGGAG |
| | | GCTTTTCCCA TCAGCGTGCA TTCACCAGCC CATCGCCACA AACTCTATCG |
| | | GAGAACATGA ATCAAAAGTG CCTCAAGAGG AATGAAAAAA GCTTTACTGG |
| | | GGCTGGGGAA GGAAGCCGGG GAAGAGATCC AAAGACTCTT GGGAGGGAGT |
| | | TACTGAAGTC TTACTACAGA AATGAGGAGG ATGCTAAAAA TGTCACGAAT |
| | | ATGGACATAT CATCTGTGGA CTGACCTTGT AAAAGACAGT GTATGTAGAA |
| | | GCATGAAGTC TTAAGGACAA AGTGCCAAAG AAAGTGGTCT TAAGAAATGT |
| | | ATAAACTTTA GAGTAGAGTT TGGAATCCCA CTAATGCAAA CTGGGATGAA |
| | | ACTAAAGCAA TAGAAACAAC ACAGTTTTGA CCTAACATAC CGTTTATAAT |
| | | GCCATTTTAA GGAAAACTAC CTGTATTTAA AAATAGAAAC ATATCAAAAA |
| | | CAAGAGAAAA GACACGAGAG AGACTGTGGC CCATCAACAG ACGTTGATAT |
| | | GCAACTGCAT GGCATGTGCT GTTTTGGTTG AAATCAAATA CATTCCGTTT |
| | | GATGGACAGC TGTCAGCTTT CTCAAACTGT GAAGATGACC CAAAGTTTCC |
| | | AACTCCTTTA CAGTATTACC GGGACTATGA ACTAAAAGGT GGGACTGAGG |
| | | ATGTGTATAG AGTGAGCGTG TGATTGTAGA CAGAGGGGTG AAGAAGGAGG |
| | | AGGAAGAGGC AGAGAAGGAG GAGACCAGGG CTGGGAAAGA AACTTCTCAA |
| | | GCAATGAAGA CTGGACTCAG GACATTTGGG GACTGTGTAC AATGAGTTAT |
| | | GGAGACTCGA GGGTTCATGC AGTCAGTGTT ATACCAAACC CAGTGTTAGG |
| | | AGAAAGGACA CAGCGTAATG GAGAAAGGGG AAGTAGTAGA ATTCAGAAAC |
| | | AAAAATGCGC ATCTCTTTCT TTGTTTGTCA AATGAAAATT TTAACTGGAA |
| | | TTGTCTGATA TTTAAGAGAA ACATTCAGGA CCTCATCATT ATGTGGGGGC |
| | | TTTGTTCTCC ACAGGGTCAG GTAAGAGATG GCCTTCTTGG CTGCCACAAT |
| | | CAGAAATCAC GCAGGCATTT TGGGTAGGCG GCCTCCAGTT TTCCTTTGAG |
| | | TCGCGAACGC TGTGCGTTTG TCAGAATGAA GTATACAAGT CAATGTTTTT |
| | | CCCCCTTTTT ATATAATAAT TATATAACTT ATGCATTTAT ACACTACGAG |
| | | TTGATCTCGG CCAGCCAAAG ACACACGACA AAAGAGACAA TCGATATAAT |
| | | GTGGCCTTGA ATTTTAACTC TGTATGCTTA ATGTTTACAA TATGAAGTTA |
| | | TTAGTTCTTA GAATGCAGAA TGTATGTAAT AAAATAAGCT TGGCCTAGCA |
| | | TGGCAAATCA GATTTATACA GGAGTCTGCA TTTGCACTTT TTTTAGTGAC |
| | | TAAAGTTGCT TAATGAAAAC ATGTGCTGAA TGTTGTGGAT TTTGTGTTAT |
| | | AATTTACTTT GTCCAGGAAC TTGTGCAAGG GAGAGCCAAG GAAATAGGAT |
| | | GTTTGGCACC CAAATGGCGT CAGCCTCTCC AGGTCCTTCT TGCCTCCCCT |
| | | CCTGTCTTTT ATTTCTAGCC CCTTTTGGAA CAGAAGGACC CCGGGTTTCA |
| | | CATTGGAGCC TCCATATTTA TGCCTGGAAT GGAAAGAGGC CTATGAAGCT |
| | | GGGGTTGTCA TTGAGAAATT CTAGTTCAGC ACCTGGTCAC AAATCACCCT |
| | | TAATTCCTGC TATGATTAAA ATACATTTGT TGAACAGTGA ACAAGCTACC |
| | | ACTCGTAAGG CAAACTGTAT TATTACTGGC AAATAAAGCG TCATGGATAG |
| | | CTGCAATTTC TCACTTTACA GAAACAAGGG ATAACGTCTA GATTTGCTGC |
| | | GGGGTTTCTC TTTCAGGAGC TCTCACTAGG TAGACAGCTT TAGTCCTGCT |
| | | ACATCAGAGT TACCTGGGCA CTGTGGCTTG GGATTCACTA GCCCTGAGCC |
| | | TGATGTTGCT GGCTATCCCT TGAAGACAAT GTTTATTTCC ATAATCTAGA |
| | | GTCAGTTTCC CTGGGCATCT TTTCTTTGAA TCACAAATGC TGCCAACCTT |
| | | GGTCCAGGTG AAGGCAACTC AAAAGGTGAA AATACAAGGT GACCGTGCGA |
| | | AGGCGCTAGC CGAAACATCT TAGCTGAATA GGTTTCTGAA CTGGCCCTTT |
| | | TCATAGCTGT TTCAGGGCCT GTTTTTTTCA CGTTGCAGTC CTTTTGCTAT |
| | | GATTATGTGA AGTTGCCAAA CCTCTGTGCT GTGGATGTTT TGGCAGTGGG |
| | | CTTTGAAGTC GGCAGGACAC GATTACCAAT GCTCCTGACA CCCCGTGTCA |
| | | TTTGGATTAG ACGGAGCCCA ACCATCCATC ATTTTGCAGC AGCCTGGGAA |
| | | GGCCCACAAA GTGCCCGTAT CTCCTTAGGG AAAATAAATA AATACAATCA |
| | | TGAAAGCTGG CAGTTAGGCT GACCCAAACT GTGCTAATGG AAAAGATCAG |
| | | TCATTTTTAT TTTGGAATGC AAAGTCAAGA CACACCTACA TTCTTCATAG |
| | | AAATACACAT TTACTTGGAT AATCACTCAG TTCTCTCTTC AAGACTGTCT |
| | | CATGAGCAAG ATCATAAAAA CAAGACATGA TTATCATATT CAATTTTAAC |
| | | AGATGTTTTC CATTAGATCC CTCAACCCTC CACCCCCAGT CCAGGTTATT |
| | | AGCAAGTCTT ATGAGCAACT GGGATAATTT TGGATAACAT GATAATACTG |
| | | AGTTCCTTCA AATACATAAT TCTTAAATTG TTTCAAAATG GCATTAACTC |
| | | TCTGTTACTG TTGTAATCTA ATTCCAAAGC CCCCTCCAGG TCATATTCAT |
| | | AATTGCATGA ACCTTTTCTC TCTGTTTGTC CCTGTCTCTT GGCTTGCCCT |
| | | GATGTATACT CAGACTCCTG TACAATCTTA CTCCTGCTGG CAAGAGATTT |
| | | GTCTTCTTTT CTTGTCTTCA ATTGGCTTTC GGGCCTTGTA TGTGGTAAAA |
| | | TCACCAAATC ACAGTCAAGA CTGTGTTTTT GTTCCTAGTT TGATGCCCTT |
| | | ATGTCCCGGA GGGGTTCACA AAGTGCTTTG TCAGGACTGC TGCAGTTAGA |
| | | AGGCTCACTG CTTCTCCTAA GCCTTCTGCA CAGATGTGGC ACCTGCAACC |
| | | CAGGAGCAGG AGCCGGAGGA GCTGCCCTCT GACAGCAGGT GCAGCAGAGA |
| | | TGGCTACAGC TCAGGAGCTG GGAAGGTGAT GGGGCACAGG GAAAGCACAG |
| | | ATGTTCTGCA GCGCCCCAAA GTGACCCATT GCCTGGAGAA AGAGAAGAAA |
| | | ATATTTTTTA AAAAGCTAGT TTATTTAGCT TCTCATTAAT TCATTCAAAT |
| | | AAAGTCGTGA GGTGACTAAT TAGAGAATAA AAATTACTTT GGACTACTCA |
| | | AAAA |
| 16 | KLK4 | AGGCAGCAGG CTGGAGCTCA GCCCAGCAGT GGAATCCAGG AGCCCAGAGG |
| | | TGGCCGGGTG CTGACGTGAT GGCCACAGCA GGAAATCCCT GGGGCTGGTT |
| | | CCTGGGGTAC CTCATCCTTG GTGTCGCAGG ATCGCTCGTC TCTGGTAGCT |
| | | GCAGCCAAAT CATAAACGGC GAGGACTGCA GCCCGCACTC GCAGCCCTGG |
| | | CAGGCGGCAC TGGTCATGGA AAACGAATTG TTCTGCTCGG GCGTCCTGGT |
| | | GCATCCGCAG TGGGTGCTGT CAGCCGCACA CTGITTCCAG AACTCCTACA |
| | | CCATCGGGCT GGGCCTGCAC AGTCTTGAGG CCGACCAAGA GCCAGGGAGC |
| | | CAGATGGTGG AGGCCAGCCT CTCCGTACGG CACCCAGAGT ACAACAGACC |
| | | CTTGCTCGCT AACGACCTCA TGCTCATCAA GTTGGACGAA TCCGTGTCCG |
| | | AGTCTGACAC CATCCGGAGC ATCAGCATTG CTTCGCAGTG CCCTACCGCG |
| | | GGGAACTCTT GCCTCGTTTC TGGCTGGGGT CTGCTGGCGA ACGGCAGAAT |
| | | GCCTACCGTG CTGCAGTGCG TGAACGTGTC GGTGGTGTCT GAGGAGGTCT |
| | | GCAGTAAGCT CTATGACCCG CTGTACCACC CCAGCATGTT CTGCGCCGGC |
| | | GGAGGGCAAG ACCAGAAGGA CTCCTGCAAC GGTGACTCTG GGGGGCCCCT |
| | | GATCTGCAAC GGGTACTTGC AGGGCCTTGT GTCTTTCGGA AAAGCCCCGT |
| | | GTGGCCAAGT TGGCGTGCCA GGTGTCTACA CCAACCTCTG CAAATTCACT |
| | | GAGTGGATAG AGAAAACCGT CCAGGCCAGT TAACTCTGGG GACTGGGAAC |
| | | CCATGAAATT GACCCCCAAA TACATCCTGC GGAAGGAATT CAGGAATATC |
| | | TGTTCCCAGC CCCTCCTCCC TCAGGCCCAG GAGTCCAGGC CCCCAGCCCC |
| | | TCCTCCCTCA AACCAAGGGT ACAGATCCCC AGCCCCTCCT CCCTCAGACC |
| | | CAGGAGTCCA GACCCCCCAG CCCCTCCTCC CTCAGACCCA GGAGTCCAGC |
| | | CCCTCCTCCC TCAGACCCAG GAGTCCAGAC CCCCCAGCCC CTCCTCCCTC |
| | | AGACCCAGGA GTCCAGCCCC TCCTCCCTCA GACCCAGGAG TCCAGACCCC |
| | | CCAGCCCCTC CTCCCTCAGA CCCAGGGGTC CAGGCCCCCA ACCCCTCCTC |
| | | CCTCAGACTC AGAGGTCCAG GCCCCCAACC CCTCCTTCCC CAGACCCAGA |
| | | GGTCCAGGTC CCAGCCCCTC CTCCCTCAGA CCCAGCGGTC CAATGCCACC |
| | | TAGACTCTCC CTGTACACAG TGCCCCCTTG TGGCACGTTG ACCCAACCTT |
| | | ACCAGTTGGT TTTTCATTTT TTGTCCCTTT CCCCTAGATC CAGAAATAAA |
| | | GTCTAAGAGA AGCGCA |
| 17 | HOXC6 | ATAACCATCT AGTTCCGAGT ACAAACTGGA GACAGAAATA AATATTAAAG |
| | | AAATCATAGA CCGACCAGGT AAAGGCAAAG GGATGAATTC CTACTTCACT |
| | | AACCCTTCCT TATCCTGCCA CCTCGCCGGG GGCCAGGACG TCCTCCCCAA |
| | | CGTCGCCCTC AATTCCACCG CCTATGATCC AGTGAGGCAT TTCTCGACCT |
| | | ATGGAGCGGC CGTTGCCCAG AACCGGATCT ACTCGACTCC CTTTTATTCG |
| | | CCACAGGAGA ATGTCGTGTT CAGTTCCAGC CGGGGGCCGT ATGACTATGG |
| | | ATCTAATTCC TTTTACCAGG AGAAAGACAT GCTCTCAAAC TGCAGACAAA |
| | | ACACCTTAGG ACATAACACA CAGACCTCAA TCGCTCAGGA TTTTAGTTCT |
| | | GAGCAGGGCA GGACTGCGCC CCAGGACCAG AAAGCCAGTA TCCAGATTTA |
| | | CCCCTGGATG CAGCGAATGA ATTCGCACAG TGGGGTCGGC TACGGAGCGG |
| | | ACCGGAGGCG CGGCCGCCAG ATCTACTCGC GGTACCAGAC CCTGGAACTG |
| | | GAGAAGGAAT TTCACTTCAA TCGCTACCTA ACGCGGCGCC GGCGCATCGA |
| | | GATCGCCAAC GCGCTTTGCC TGACCGAGCG ACAGATCAAA ATCTGGTTCC |
| | | AGAACCGCCG GATGAAGTGG AAAAAAGAAT CTAATCTCAC ATCCACTCTC |
| | | TCGGGGGGCG GCGGAGGGGC CACCGCCGAC AGCCTGGGCG GAAAAGAGGA |
| | | AAAGCGGGAA GAGACAGAAG AGGAGAAGCA GAAAGAGTGA CCAGGACTGT |
| | | CCCTGCCACC CCTCTCTCCC TTTCTCCCTC GCTCCCCACC AACTCTCCCC |
| | | TAATCACACA CTCTGTATTT ATCACTGGCA CAATTGATGT GTTTTGATTC |
| | | CCTAAAACAA AATTAGGGAG TCAAACGTGG ACCTGAAAGT CAGCTCTGGA |
| | | CCCCCTCCCT CACCGCACAA CTCTCTTTCA CCACGCGCCT CCTCCTCCTC |
| | | GCTCCCTTGC TAGCTCGTTC TCGGCTTGTC TACAGGCCCT TTTCCCCGTC |
| | | CAGGCCTTGG GGGCTCGGAC CCTGAACTCA GACTCTACAG ATTGCCCTCC |
| | | AAGTGAGGAC TTGGCTCCCC CACTCCTTCG ACGCCCCCAC CCCCGCCCCC |
| | | CGTGCAGAGA GCCGGCTCCT GGGCCTGCTG GGGCCTCTGC TCCAGGGCCT |
| | | CAGGGCCCGG CCTGGCAGCC GGGGAGGGCC GGAGGCCCAA GGAGGGCGCG |
| | | CCTTGGCCCC ACACCAACCC CCAGGGCCTC CCCGCAGTCC CTGCCTAGCC |
| | | CCTCTGCCCC AGCAAATGCC CAGCCCAGGC AAATTGTATT TAAAGAATCC |
| | | TGGGGGTCAT TATGGCATTT TACAAACTGT GACCGTTTCT GTGTGAAGAT |
| | | TTTTAGCTGT ATTTGTGGTC TCTGTATTTA TATTTATGTT TAGCACCGTC |
| | | AGTGTTCCTA TCCAATTTCA AAAAAGGAAA AAAAAGAGGG AAAATTACAA |
| | | AAAGAGAGAA AAAAAGTGAA TGACGTTTGT TTAGCCAGTA GGAGAAAATA |
| | | AATAAATAAA TAAATCCCTT CGTGTTACCC TCCTGTATAA ATCCAACCTC |
| | | TGGGTCCGTT CTCGAATATT TAATAAAACT GATATTATTT TTAAAACTTT |
| | | A |
| 18 | KLK3 | AGCCCCAAGC TTACCACCTG CACCCGGAGA GCTGTGTCAC CATGTGGGTC |
| | | CCGGTTGTCT TCCTCACCCT GTCCGTGACG TGGATTGGTG CTGCACCCCT |
| | | CATCCTGTCT CGGATTGTGG GAGGCTGGGA GTGCGAGAAG CATTCCCAAC |
| | | CCTGGCAGGT GCTTGTGGCC TCTCGTGGCA GGGCAGTCTG CGGCGGTGTT |
| | | CTGGTGCACC CCCAGTGGGT CCTCACAGCT GCCCACTGCA TCAGGAACAA |
| | | AAGCGTGATC TTGCTGGGTC GGCACAGCCT GTTTCATCCT GAAGACACAG |
| | | GCCAGGTATT TCAGGTCAGC CACAGCTTCC CACACCCGCT CTACGATATG |
| | | AGCCTCCTGA AGAATCGATT CCTCAGGCCA GGTGATGACT CCAGCCACGA |
| | | CCTCATGCTG CTCCGCCTGT CAGAGCCTGC CGAGCTCACG GATGCTGTGA |
| | | AGGTCATGGA CCTGCCCACC CAGGAGCCAG CACTGGGGAC CACCTGCTAC |
| | | GCCTCAGGCT GGGGCAGCAT TGAACCAGAG GAGTTCTTGA CCCCAAAGAA |
| | | ACTTCAGTGT GTGGACCTCC ATGTTATTTC CAATGACGTG TGTGCGCAAG |
| | | TTCACCCTCA GAAGGTGACC AAGTTCATGC TGTGTGCTGG ACGCTGGACA |
| | | GGGGGCAAAA GCACCTGCTC GGGTGATTCT GGGGGCCCAC TTGTCTGTAA |
| | | TGGTGTGCTT CAAGGTATCA CGTCATGGGG CAGTGAACCA TGTGCCCTGC |
| | | CCGAAAGGCC TTCCCTGTAC ACCAAGGTGG TGCATTACCG GAAGTGGATC |
| | | AAGGACACCA TCGTGGCCAA CCCCTGAGCA CCCCTATCAA CCCCCTATTG |
| | | TAGTAAACTT GGAACCTTGG AAATGACCAG GCCAAGACTC AAGCCTCCCC |
| | | AGTTCTACTG ACCTTTGTCC TTAGGTGTGA GGTCCAGGGT TGCTAGGAAA |
| | | AGAAATCAGC AGACACAGGT GTAGACCAGA GTGTTTCTTA AATGGTGTAA |
| | | TTTTGTCCTC TCTGTGTCCT GGGGAATACT GGCCATGCCT GGAGACATAT |
| | | CACTCAATTT CTCTGAGGAC ACAGATAGGA TGGGGTGTCT GTGTTATTTG |
| | | TGGGGTACAG AGATGAAAGA GGGGTGGGAT CCACACTGAG AGAGTGGAGA |
| | | GTGACATGTG CTGGACACTG TCCATGAAGC ACTGAGCAGA AGCTGGAGGC |
| | | ACAACGCACC AGACACTCAC AGCAAGGATG GAGCTGAAAA CATAACCCAC |
| | | TCTGTCCTGG AGGCACTGGG AAGCCTAGAG AAGGCTGTGA GCCAAGGAGG |
| | | GAGGGTCTTC CTTTGGCATG GGATGGGGAT GAAGTAAGGA GAGGGACTGG |
| | | ACCCCCTGGA AGCTGATTCA CTATGGGGGG AGGTGTATTG AAGTCCTCCA |
| | | GACAACCCTC AGATTTGATG ATTTCCTAGT AGAACTCACA GAAATAAAGA |
| | | GCTGTTATAC TGTG |
| 19 | ACSM1 | ATACCTACCAGGGACCTAGTTCAGCTACGTCCTAAACAGTACCAGGCAGGTGGTGA |
| | | CTTGAGAACTCTGTGCCTGGTTTCTGAGGACTGTTTCACCATGCAGTGGCTAATGA |
| | | GGTTCCGGACCCTCTGGGGCATCCACAAATCCTTCCACAACATCCACCCTGCCCCT |
| | | TCACAGCTGCGCTGCCGGTCTTTATCAGAATTTGGAGCCCCAAGATGGAATGACTA |
| | | TGAAGTACCGGAGGAATTTAACTTTGCAAGTTATGTACTGGACTACTGGGCTCAAA |
| | | AGGAGAAGGAGGGCAAGAGAGGTCCAAATCCAGCTTTTTGGTGGGTGAATGGCCAA |
| | | GGGGATGAAGTAAAGTGGAGCTTCAGAGAGATGGGAGACCTAACCCGCCGTGTAGC |
| | | CAACGTCTTCACACAGACCTGTGGCCTACAACAGGGAGACCATCTGGCCTTGATGC |
| | | TGCCTCGAGTTCCTGAGTGGTGGCTGGTGGCTGTGGGCTGCATGCGAACAGGGATC |
| | | ATCTTCATTCCTGCGACCATCCTGTTGAAGGCCAAAGACATTCTCTATCGACTACA |
| | | GTTGTCTAAAGCCAAGGGCATTGTGACCATAGATGCCCTTGCCTCAGAGGTGGACT |
| | | CCATAGCTTCTCAGTGCCCCTCTCTGAAAACCAAGCTCCTGGTGTCTGATCACAGC |
| | | CGTGAAGGGTGGCTGGACTTCCGATCGCTGGTTAAATCAGCATCCCCAGAACACAC |
| | | CTGTGTTAAGTCAAAGACCTTGGACCCAATGGTCATCTTCTTCACCAGTGGGACCA |
| | | CAGGCTTCCCCAAGATGGCAAAACACTCCCATGGGTTGGCCTTACAACCCTCCTTC |
| | | CCAGGAAGTAGGAAATTACGGAGCCTGAAGACATCTGATGTCTCCTGGTGCCTGTC |
| | | GGACTCAGGATGGATTGTGGCTACCATTTGGACCCTGGTAGAACCATGGACAGCGG |
| | | GTTGTACAGTCTTTATCCACCATCTGCCACAGTTTGACACCAAGGTCATCATACAG |
| | | ACATTGTTGAAATACCCCATTAACCACTTTTGGGGGGTATCATCTATATATCGAAT |
| | | GATTCTGCAGCAGGATTTCACCAGCATCAGGTTCCCTGCCCTGGAGCACTGCTATA |
| | | CTGGCGGGGAGGTCGTGTTGCCCAAGGATCAGGAGGAGTGGAAAAGACGGACGGGC |
| | | CTTCTGCTCTACGAGAACTATGGGCAGTCGGAAACGGGACTAATTTGTGCCACCTA |
| | | CTGGGGAATGAAGATCAAGCCGGGTTTCATGGGGAAGGCCACTCCACCCTACGACG |
| | | TCCAGGTCATTGATGACAAGGGCAGCATCCTGCCACCTAACACAGAAGGAAACATT |
| | | GGCATCAGAATCAAACCTGTCAGGCCTGTGAGCCTCTTCATGTGCTATGAGGGTGA |
| | | CCCAGAGAAGACAGCTAAAGTGGAATGTGGGGACTTCTACAACACTGGGGACAGAG |
| | | GTAAGATGGATGAAGAGGGCTACATTTGTTTCCTGGGGAGGAGTGATGACATCATT |
| | | AATGCCTCTGGGTATCGCATCGGGCCTGCAGAGGTTGAAAGCGCTTTGGTGGAGCA |
| | | CCCAGCGGTGGCGGAGTCAGCCGTGGTGGGCAGCCCAGACCCGATTCGAGGGGAGG |
| | | TGGTGAAGGCCTTTATTGTCCTGACCCCACAGTTCCTGTCCCATGACAAGGATCAG |
| | | CTGACCAAGGAACTGCAGCAGCATGTCAAGTCAGTGACAGCCCCATACAAGTACCC |
| | | AAGGAAGGTGGAGTTTGTCTCAGAGCTGCCAAAAACCATCACTGGCAAGATTGAAC |
| | | GGAAGGAACTTCGGAAAAAGGAGACTGGTCAGATGTAATCGGCAGTGAACTCAGAA |
| | | CGCACTGCACACCTAAGGCAAATCCCTGGCCACTTTAGTCTCCCCACTATGGTGAG |
| | | GACGAGGGTGGGGCATTGAGAGTGTTGATTTGGGAAAGTATCAGGAGTGCCATGAT |
| | | TCCAATGTTTTCCTTCTTTTAAATTAAATTCAGTTGCTCTGCTTCCTCCAAGTCCT |
| | | CTGTATCTTTAGAATTTCCCAGGTGAGCACTCATAACGCAAGTAATAAAATACTGA |
| | | TATCAACAA |
| 20 | AMACR | AGTTTCCTTCAGCGGGGCACTGGGAAGCGCCATGGCACTGCAGGGCATCTCGGTCG |
| | | TGGAGCTGTCCGGCCTGGCCCCGGGCCCGTTCTGTGCTATGGTCCTGGCTGACTTC |
| | | GGGGCGCGTGTGGTACGCGTGGACCGGCCCGGCTCCCGCTACGACGTGAGCCGCTT |
| | | GGGCCGGGGCAAGCGCTCGCTAGTGCTGGACCTGAAGCAGCCGCGGGGAGCCGCCG |
| | | TGCTGCGGCGTCTGTGCAAGCGGTCGGATGTGCTGCTGGAGCCCTTCCGCCGCGGT |
| | | GTCATGGAGAAACTCCAGCTGGGCCCAGAGATTCTGCAGCGGGAAAATCCAAGGCT |
| | | TATTTATGCCAGGCTGAGTGGATTTGGCCAGTCAGGAAGCTTCTGCCGGTTAGCTG |
| | | GCCACGATATCAACTATTTGGCTTTGTCAGGTGTTCTCTCAAAAATTGGCAGAAGT |
| | | GGTGAGAATCCGTATGCCCCGCTGAATCTCCTGGCTGACTTTGCTGGTGGTGGCCT |
| | | TATGTGTGCACTGGGCATTATAATGGCTCTTTTTGACCGCACACGCACTGGCAAGG |
| | | GTCAGGTCATTGATGCAAATATGGTGGAAGGAACAGCATATTTAAGTTCTTTTCTG |
| | | TGGAAAACTCAGAAATTGAGTCTGTGGGAAGCACCTCGAGGACAGAACATGTTGGA |
| | | TGGTGGAGCACCTTTCTATACGACTTACAGGACAGCAGATGGGGAATTCATGGCTG |
| | | TTGGAGCAATAGAACCCCAGTTCTACGAGCTGCTGATCAAAGGACTTGGACTAAAG |
| | | TCTGATGAACTTCCCAATCAGATGAGCATGGATGATTGGCCAGAAATGAAGAAGAA |
| | | GTTTGCAGATGTATTTGCAGAGAAGACGAAGGCAGAGTGGTGTCAAATCTTTGACG |
| | | GCACAGATGCCTGTGTGACTCCGGTTCTGACTTTTGAGGAGGTTGTTCATCATGAT |
| | | CACAACAAGGAACGGGGCTCGTTTATCACCAGTGAGGAGCAGGACGTGAGCCCCCG |
| | | CCCTGCACCTCTGCTGTTAAACACCCCAGCCATCCCTTCTTTCAAAAGGGATCCTT |
| | | TCATAGGAGAACACACTGAGGAGATACTTGAAGAATTTGGATTCAGCCGCGAAGAG |
| | | ATTTATCAGCTTAACTCAGATAAAATCATTGAAAGTAATAAGGTAAAAGCTAGTCT |
| | | CTAACTTCCAGGCCCACGGCTCAAGTGAATTTGAATACTGCATTTACAGTGTAGAG |
| | | TAACACATAACATTGTATGCATGGAAACATGGAGGAACAGTATTACAGTGTCCTAC |
| | | CACTCTAATCAAGAAAAGAATTACAGACTCTGATTCTACAGTGATGATTGAATTCT |
| | | AAAAATGGTTATCATTAGGGCTTTTGATTTATAAAACTTTGGGTACTTATACTAAA |
| | | TTATGGTAGTTATTCTGCCTTCCAGTTTGCTTGATATATTTGTTGATATTAAGATT |
| | | CTTGACTTATATTTTGAATGGGTTCTAGTGAAAAAGGAATGATATATTCTTGAAGA |
| | | CATCGATATACATTTATTTACACTCTTGATTCTACAATGTAGAAAATGAGGAAATG |
| | | CCACAAATTGTATGGTGATAAAAGTCACGTGAAACAGAGTGATTGGTTGCATCCAG |
| | | GCCTTTTGTCTTGGTGTTCATGATCTCCCTCTAAGCACATTCCAAACTTTAGCAAC |
| | | AGTTATCACACTTTGTAATTTGCAAAGAAAAGTTTCACCTGTATTGAATCAGAATG |
| | | CCTTCAACTGAAAAAAACATATCCAAAATAATGAGGAAATGTGTTGGCTCACTACG |
| | | TAGAGTCCAGAGGGACAGTCAGTTTTAGGGTTGCCTGTATCCAGTAACTCGGGGCC |
| | | TGTTTCCCCGTGGGTCTCTGGGCTGTCAGCTTTCCTTTCTCCATGTGTTTGATTTC |
| | | TCCTCAGGCTGGTAGCAAGTTCTGGATCTTATACCCAACACACAGCAACATCCAGA |
| | | AATAAAGATCTCAGGACCCCCCAGCAAGTCGTTTTGTGTCTCCTTGGACTGAGTTA |
| | | AGTTACAAGCCTTTCTTATACCTGTCTTTGACAAAGAAGACGGGATTGTCTTTACA |
| | | TAAAACCAGCCTGCTCCTGGAGCTTCCCTGGACTCAACTTCCTAAAGGCATGTGAG |
| | | GAAGGGGTAGATTCCACAATCTAATCCGGGTGCCATCAGAGTAGAGGGAGTAGAGA |
| | | ATGGATGTTGGGTAGGCCATCAATAAGGTCCATTCTGCGCAGTATCTCAACTGCCG |
| | | TTCAACAATCGCAAGAGGAAGGTGGAGCAGGTTTCTTCATCTTACAGTTGAGAAAA |
| | | CAGAGACTCAGAAGGGCTTCTTAGTTCATGTTTCCCTTAGCGCCTCAGTGATTTTT |
| | | TCATGGTGGCTTAGGCCAAAAGAAATATCTAACCATTCAATTTATAAATAATTAGG |
| | | TCCCCAACGAATTAAATATTATGTCCTACCAACTTATTAGCTGCTTGAAAAATATA |
| | | ATACACATAAATAAAAAAATATATTTTTCATTTCTATTTCATTGTTAATCACAACT |
| | | ACTTACTAAGGAGATGTATGCACCTATTGGACACTGTGCAACTTCTCACCTGGAAT |
| | | GAGATTGGACACTGCTGCCCTCATTTTCTGCTCCATGTTGGTGTCCATATAGTACT |
| | | TGATTTTTTATCAGATGGCCTGGAAAACCCAGTCTCACAAAAATATGAAATTATCA |
| | | GAAGGATTATAGTGCAATCTTATGTTGAAAGAATGAACTACCTCACTAGTAGTTCA |
| | | CGTGATGTCTGACAGATGTTGAGTTTCATTGTGTTTGTGTGTTCAAATTTTTAAAT |
| | | ATTCTGAGATACTCTTGTGAGGTCACTCTAATGCCCTGGGTGCCTTGGCACAGTTT |
| | | TAGAAATACCAGTTGAAAATATTTGCTCAGGAATATGCAACTAGGAAGGGGCAGAA |
| | | TCAGAATTTAAGCTTTCATATTCTAGCCTTCAGTCTTGTTCTTCAACCATTTTTAG |
| | | GAACTTTCCCATAAGGTTATGTTTTCCAGCCCAGGCATGGAGGATCACTTGAGGCC |
| | | AAGAGTTCGAGACCAGCCTGGGGAACTTGGCTGGACCTCCGTTTCTACGAAATAAA |
| | | AATAAAAAAATTATCCAGGTATGGTGGTGTGTGCCTGTAGTCCTATCTACTCAAGG |
| | | GTGGGGCAGGAGGATCACTTGAGCCCAGGAATTTGAGGCCACAGTGAATTAGGATT |
| | | GCACCACTGCACTCTAGCCCAGGCAACAGAACAAGAACCTGTCTCTAAATAAATAA |
| | | ATAAAAATAATAATAATAAAAAAGATGTTTTCCCTACAACTCAGACTTTTCATTTG |
| | | AACTCGGTCCAGCAAGGAAAATATAACCCACTCGAAGTCTTTAAAACAGAGGAAAT |
| | | TTAATATAAAGAATTCCACTGGTGACGAAAGAGCAGAGAAGCCCAGAAGATAGTGA |
| | | GGCAACCCTGATAGGAACATAACTAGGAAGCCAAGACCACTCCTATGGTTGCAGGG |
| | | GTGATGGGAAAGCTGGTGTACTTGGACCCAGAAGCCAAAGTTGCTGCACCCACCTT |
| | | GGAGACATAGACACTGGCAGTAATACCTCAGGGAGAAGAAAGAAATCTAGGGAAAT |
| | | ATCCTGGCTTCTTTCCTTCTCTCTCTCCCCTAGTCTTCCTACCAGTGTCTCCCATT |
| | | AGCCAAATCTACCTAGAAGCCAGAAAACAAGGGAACCCTGGAAATGTAGCCCCATA |
| | | AGATAAAGAGCACCAAAGGAAATAGATCTGAGCAGACAGGCAGCACAAAATGCAGT |
| | | GTGTATGGTTTATTCACTCAGTAATTCCTTTAGCAAATGTTTATTGAGGATCTACT |
| | | AGGTGCCAGGTATCATGATACTTGCTGGGGATACCATAATGAACAAAACAGACCTG |
| | | TTCTCCGCTCTTGAGGAAATCAAAGACAAACACAGGATATGGAATAAACCCAGAAT |
| | | TATCTCATTGTAAAATGTGTTAAGTACCACGAGGAGAAATATCAGGGCGATCTGAC |
| | | ACAGCTAATGATTTGAAGAAGGGTGTGACCTGCCACCATTTTAAATCTAGTTATTT |
| | | CACTCCTGAGCTGTGTGTGTGGAAAACTTGTAGTAAAAAATAGAATGTCTATATTT |
| | | ATAAAAAGTTTATGAAAAGA |
| 21 | AR | AGCGCCCCCTCCGAGATCCCGGGGAGCCAGCTTGCTGGGAGAGCGGGACGGTCCGG |
| | | AGCAAGCCCAGAGGCAGAGGAGGCGACAGAGGGAAAAAGGGCCGAGCTAGCCGCTC |
| | | CAGTGCTGTACAGGAGCCGAAGGGACGCACCACGCCAGCCCCAGCCCGGCTCCAGC |
| | | GACAGCCAACGCCTCTTGCAGCGCGGCGGCTTCGAAGCCGCCGCCCGGAGCTGCCC |
| | | TTTCCTCTTCGGTGAAGTTTTTAAAAGCTGCTAAAGACTCGGAGGAAGCAAGGAAA |
| | | GTGCCTGGTAGGACTGACGGCTGCCTTTGTCCTCCTCCTCTCCACCCCGCCTCCCC |
| | | CCACCCTGCCTTCCCCCCCTCCCCCGTCTTCTCTCCCGCAGCTGCCTCAGTCGGCT |
| | | ACTCTCAGCCAACCCCCCTCACCACCCTTCTCCCCACCCGCCCCCCCGCCCCCGTC |
| | | GGCCCAGCGCTGCCAGCCCGAGTTTGCAGAGAGGTAACTCCCTTTGGCTGCGAGCG |
| | | GGCGAGCTAGCTGCACATTGCAAAGAAGGCTCTTAGGAGCCAGGCGACTGGGGAGC |
| | | GGCTTCAGCACTGCAGCCACGACCCGCCTGGTTAGGCTGCACGCGGAGAGAACCCT |
| | | CTGTTTTCCCCCACTCTCTCTCCACCTCCTCCTGCCTTCCCCACCCCGAGTGCGGA |
| | | GCCAGAGATCAAAAGATGAAAAGGCAGTCAGGTCTTCAGTAGCCAAAAAACAAAAC |
| | | AAACAAAAACAAAAAAGCCGAAATAAAAGAAAAAGATAATAACTCAGTTCTTATTT |
| | | GCACCTACTTCAGTGGACACTGAATTTGGAAGGTGGAGGATTTTGTTTTTTTCTTT |
| | | TAAGATCTGGGCATCTTTTGAATCTACCCTTCAAGTATTAAGAGACAGACTGTGAG |
| | | CCTAGCAGGGCAGATCTTGTCCACCGTGTGTCTTCTTCTGCACGAGACTTTGAGGC |
| | | TGTCAGAGCGCTTTTTGCGTGGTTGCTCCCGCAAGTTTCCTTCTCTGGAGCTTCCC |
| | | GCAGGTGGGCAGCTAGCTGCAGCGACTACCGCATCATCACAGCCTGTTGAACTCTT |
| | | CTGAGCAAGAGAAGGGGAGGCGGGGTAAGGGAAGTAGGTGGAAGATTCAGCCAAGC |
| | | TCAAGGATGGAAGTGCAGTTAGGGCTGGGAAGGGTCTACCCTCGGCCGCCGTCCAA |
| | | GACCTACCGAGGAGCTTTCCAGAATCTGTTCCAGAGCGTGCGCGAAGTGATCCAGA |
| | | ACCCGGGCCCCAGGCACCCAGAGGCCGCGAGCGCAGCACCTCCCGGCGCCAGTTTG |
| | | CTGCTGCTGCAGCAGCAGCAGCAGCAGCAGCAGCAGCAGCAGCAGCAGCAGCAGCA |
| | | GCAGCAGCAGCAGCAGCAGCAAGAGACTAGCCCCAGGCAGCAGCAGCAGCAGCAGG |
| | | GTGAGGATGGTTCTCCCCAAGCCCATCGTAGAGGCCCCACAGGCTACCTGGTCCTG |
| | | GATGAGGAACAGCAACCTTCACAGCCGCAGTCGGCCCTGGAGTGCCACCCCGAGAG |
| | | AGGTTGCGTCCCAGAGCCTGGAGCCGCCGTGGCCGCCAGCAAGGGGCTGCCGCAGC |
| | | AGCTGCCAGCACCTCCGGACGAGGATGACTCAGCTGCCCCATCCACGTTGTCCCTG |
| | | CTGGGCCCCACTTTCCCCGGCTTAAGCAGCTGCTCCGCTGACCTTAAAGACATCCT |
| | | GAGCGAGGCCAGCACCATGCAACTCCTTCAGCAACAGCAGCAGGAAGCAGTATCCG |
| | | AAGGCAGCAGCAGCGGGAGAGCGAGGGAGGCCTCGGGGGCTCCCACTTCCTCCAAG |
| | | GACAATTACTTAGGGGGCACTTCGACCATTICTGACAACGCCAAGGAGTIGTGTAA |
| | | GGCAGTGTCGGTGTCCATGGGCCTGGGTGTGGAGGCGTTGGAGCATCTGAGTCCAG |
| | | GGGAACAGCTTCGGGGGGATTGCATGTACGCCCCACTTTTGGGAGTTCCACCCGCT |
| | | GTGCGTCCCACTCCTTGTGCCCCATTGGCCGAATGCAAAGGTTCTCTGCTAGACGA |
| | | CAGCGCAGGCAAGAGCACTGAAGATACTGCTGAGTATTCCCCTTTCAAGGGAGGTT |
| | | ACACCAAAGGGCTAGAAGGCGAGAGCCTAGGCTGCTCTGGCAGCGCTGCAGCAGGG |
| | | AGCTCCGGGACACTTGAACTGCCGTCTACCCTGTCTCTCTACAAGTCCGGAGCACT |
| | | GGACGAGGCAGCTGCGTACCAGAGTCGCGACTACTACAACTTTCCACTGGCTCTGG |
| | | CCGGACCGCCGCCCCCTCCGCCGCCTCCCCATCCCCACGCTCGCATCAAGCTGGAG |
| | | AACCCGCTGGACTACGGCAGCGCCTGGGCGGCTGCGGCGGCGCAGTGCCGCTATGG |
| | | GGACCTGGCGAGCCTGCATGGCGCGGGTGCAGCGGGACCCGGTTCTGGGTCACCCT |
| | | CAGCCGCCGCTTCCTCATCCTGGCACACTCTCTTCACAGCCGAAGAAGGCCAGTTG |
| | | TATGGACCGTGTGGTGGTGGTGGGGGTGGTGGCGGCGGCGGCGGCGGCGGCGGCGG |
| | | CGGCGGCGGCGGCGGCGGCGGCGGCGAGGCGGGAGCTGTAGCCCCCTACGGCTACA |
| | | CTCGGCCCCCTCAGGGGCTGGCGGGCCAGGAAAGCGACTTCACCGCACCTGATGTG |
| | | TGGTACCCTGGCGGCATGGTGAGCAGAGTGCCCTATCCCAGTCCCACTTGTGTCAA |
| | | AAGCGAAATGGGCCCCTGGATGGATAGCTACTCCGGACCTTACGGGGACATGCGTT |
| | | TGGAGACTGCCAGGGACCATGTTTTGCCCATTGACTATTACTTTCCACCCCAGAAG |
| | | ACCTGCCTGATCTGTGGAGATGAAGCTTCTGGGTGTCACTATGGAGCTCTCACATG |
| | | TGGAAGCTGCAAGGTCTTCTTCAAAAGAGCCGCTGAAGGGAAACAGAAGTACCTGT |
| | | GCGCCAGCAGAAATGATTGCACTATTGATAAATTCCGAAGGAAAAATTGTCCATCT |
| | | TGTCGTCTTCGGAAATGTTATGAAGCAGGGATGACTCTGGGAGCCCGGAAGCTGAA |
| | | GAAACTTGGTAATCTGAAACTACAGGAGGAAGGAGAGGCTTCCAGCACCACCAGCC |
| | | CCACTGAGGAGACAACCCAGAAGCTGACAGTGTCACACATTGAAGGCTATGAATGT |
| | | CAGCCCATCTTTCTGAATGTCCTGGAAGCCATTGAGCCAGGTGTAGTGTGTGCTGG |
| | | ACACGACAACAACCAGCCCGACTCCTTTGCAGCCTTGCTCTCTAGCCTCAATGAAC |
| | | TGGGAGAGAGACAGCTTGTACACGTGGTCAAGTGGGCCAAGGCCTTGCCTGGCTTC |
| | | CGCAACTTACACGTGGACGACCAGATGGCTGTCATTCAGTACTCCTGGATGGGGCT |
| | | CATGGTGTTTGCCATGGGCTGGCGATCCTTCACCAATGTCAACTCCAGGATGCTCT |
| | | ACTTCGCCCCTGATCTGGTTTTCAATGAGTACCGCATGCACAAGTCCCGGATGTAC |
| | | AGCCAGTGTGTCCGAATGAGGCACCTCTCTCAAGAGTTTGGATGGCTCCAAATCAC |
| | | CCCCCAGGAATTCCTGTGCATGAAAGCACTGCTACTCTTCAGCATTATTCCAGTGG |
| | | ATGGGCTGAAAAATCAAAAATTCTTTGATGAACTTCGAATGAACTACATCAAGGAA |
| | | CTCGATCGTATCATTGCATGCAAAAGAAAAAATCCCACATCCTGCTCAAGACGCTT |
| | | CTACCAGCTCACCAAGCTCCTGGACTCCGTGCAGCCTATTGCGAGAGAGCTGCATC |
| | | AGTTCACTTTTGACCTGCTAATCAAGTCACACATGGTGAGCGTGGACTTTCCGGAA |
| | | ATGATGGCAGAGATCATCTCTGTGCAAGTGCCCAAGATCCTTTCTGGGAAAGTCAA |
| | | GCCCATCTATTTCCACACCCAGTGAAGCATTGGAAACCCTATTTCCCCACCCCAGC |
| | | TCATGCCCCCTTTCAGATGTCTTCTGCCTGTTATAACTCTGCACTACTCCTCTGCA |
| | | GTGCCTTGGGGAATTTCCTCTATTGATGTACAGTCTGTCATGAACATGTTCCTGAA |
| | | TTCTATTTGCTGGGCTTTTTTTTTCTCTTTCTCTCCTTTCTTTTTCTTCTTCCCTC |
| | | CCTATCTAACCCTCCCATGGCACCTTCAGACTTTGCTTCCCATTGTGGCTCCTATC |
| | | TGTGTTTTGAATGGTGTTGTATGCCTTTAAATCTGTGATGATCCTCATATGGCCCA |
| | | GTGTCAAGTTGTGCTTGTTTACAGCACTACTCTGTGCCAGCCACACAAACGTTTAC |
| | | TTATCTTATGCCACGGGAAGTTTAGAGAGCTAAGATTATCTGGGGAAATCAAAACA |
| | | AAAACAAGCAAACAAAAAAAAAAAGCAAAAACAAAACAAAAAATAAGCCAAAAAAC |
| | | CTTGCTAGTGTTTTTTCCTCAAAAATAAATAAATAAATAAATAAATACGTACATAC |
| | | ATACACACATACATACAAACATATAGAAATCCCCAAAGAGGCCAATAGTGACGAGA |
| | | AGGTGAAAATTGCAGGCCCATGGGGAGTTACTGATTTTTTCATCTCCTCCCTCCAC |
| | | GGGAGACTTTATTTTCTGCCAATGGCTATTGCCATTAGAGGGCAGAGTGACCCCAG |
| | | AGCTGAGTTGGGCAGGGGGGTGGACAGAGAGGAGAGGACAAGGAGGGCAATGGAGC |
| | | ATCAGTACCTGCCCACAGCCTTGGTCCCTGGGGGCTAGACTGCTCAACTGTGGAGC |
| | | AATTCATTATACTGAAAATGTGCTTGTTGTTGAAAATTTGTCTGCATGTTAATGCC |
| | | TCACCCCCAAACCCTTTTCTCTCTCACTCTCTGCCTCCAACTTCAGATTGACTTTC |
| | | AATAGTTTTTCTAAGACCTTTGAACTGAATGTTCTCTTCAGCCAAAACTTGGCGAC |
| | | TTCCACAGAAAAGTCTGACCACTGAGAAGAAGGAGAGCAGAGATTTAACCCTTTGT |
| | | AAGGCCCCATTTGGATCCAGGTCTGCTTTCTCATGTGTGAGTCAGGGAGGAGCTGG |
| | | AGCCAGAGGAGAAGAAAATGATAGCTTGGCTGTTCTCCTGCTTAGGACACTGACTG |
| | | AATAGTTAAACTCTCACTGCCACTACCTTTTCCCCACCTTTAAAAGACCTGAATGA |
| | | AGTTTTCTGCCAAACTCCGTGAAGCCACAAGCACCTTATGTCCTCCCTTCAGTGTT |
| | | TTGTGGGCCTGAATTTCATCACACTGCATTTCAGCCATGGTCATCAAGCCTGTTTG |
| | | CTTCTTTTGGGCATGTTCACAGATTCTCTGTTAAGAGCCCCCACCACCAAGAAGGT |
| | | TAGCAGGCCAACAGCTCTGACATCTATCTGTAGATGCCAGTAGTCACAAAGATTTC |
| | | TTACCAACTCTCAGATCGCTGGAGCCCTTAGACAAACTGGAAAGAAGGCATCAAAG |
| | | GGATCAGGCAAGCTGGGCGTCTTGCCCTTGTCCCCCAGAGATGATACCCTCCCAGC |
| | | AAGTGGAGAAGTTCTCACTTCCTTCTTTAGAGCAGCTAAAGGGGCTACCCAGATCA |
| | | GGGTTGAAGAGAAAACTCAATTACCAGGGTGGGAAGAATGAAGGCACTAGAACCAG |
| | | AAACCCTGCAAATGCTCTTCTTGTCACCCAGCATATCCACCTGCAGAAGTCATGAG |
| | | AAGAGAGAAGGAACAAAGAGGAGACTCTGACTACTGAATTAAAATCTTCAGCGGCA |
| | | AAGCCTAAAGCCAGATGGACACCATCTGGTGAGTTTACTCATCATCCTCCTCTGCT |
| | | GCTGATTCTGGGCTCTGACATTGCCCATACTCACTCAGATTCCCCACCTTTGTTGC |
| | | TGCCTCTTAGTCAGAGGGAGGCCAAACCATTGAGACTTTCTACAGAACCATGGCTT |
| | | CTTTCGGAAAGGTCTGGTTGGTGTGGCTCCAATACTTTGCCACCCATGAACTCAGG |
| | | GTGTGCCCTGGGACACTGGTTTTATATAGTCTTTTGGCACACCTGTGTTCTGTTGA |
| | | CTTCGTTCTTCAAGCCCAAGTGCAAGGGAAAATGTCCACCTACTTTCTCATCTTGG |
| | | CCTCTGCCTCCTTACTTAGCTCTTAATCTCATCTGTTGAACTCAAGAAATCAAGGG |
| | | CCAGTCATCAAGCTGCCCATTTTAATTGATTCACTCTGTTTGTTGAGAGGATAGTT |
| | | TCTGAGTGACATGATATGATCCACAAGGGTTTCCTTCCCTGATTTCTGCATTGATA |
| | | TTAATAGCCAAACGAACTTCAAAACAGCTTTAAATAACAAGGGAGAGGGGAACCTA |
| | | AGATGAGTAATATGCCAATCCAAGACTGCTGGAGAAAACTAAAGCTGACAGGTTCC |
| | | CTTTTTGGGGTGGGATAGACATGTTCTGGTTTTCTTTATTATTACACAATCTGGCT |
| | | CATGTACAGGATCACTTTTAGCTGTTTTAAACAGAAAAAAATATCCACCACTCTTT |
| | | TCAGTTACACTAGGTTACATTTTAATAGGTCCTTTACATCTGTTTTGGAATGATTT |
| | | TCATCTTTTGTGATACACAGATTGAATTATATCATTTTCATATCTCTCCTTGTAAA |
| | | TACTAGAAGCTCTCCTTTACATTTCTCTATCAAATTTTTCATCTTTATGGGTTTCC |
| | | CAATTGTGACTCTTGTCTTCATGAATATATGTTTTTCATTTGCAAAAGCCAAAAAT |
| | | CAGTGAAACAGCAGTGTAATTAAAAGCAACAACTGGATTACTCCAAATTTCCAAAT |
| | | GACAAAACTAGGGAAAAATAGCCTACACAAGCCTTTAGGCCTACTCTTTCTGTGCT |
| | | TGGGTTTGAGTGAACAAAGGAGATTTTAGCTTGGCTCTGTTCTCCCATGGATGAAA |
| | | GGAGGAGGATTTTTTTTTTCTTTTGGCCATTGATGTTCTAGCCAATGTAATTGACA |
| | | GAAGTCTCATTTTGCATGCGCTCTGCTCTACAAACAGAGTTGGTATGGTTGGTATA |
| | | CTGTACTCACCTGTGAGGGACTGGCCACTCAGACCCACTTAGCTGGTGAGCTAGAA |
| | | GATGAGGATCACTCACTGGAAAAGTCACAAGGACCATCTCCAAACAAGTTGGCAGT |
| | | GCTCGATGTGGACGAAGAGTGAGGAAGAGAAAAAGAAGGAGCACCAGGGAGAAGGC |
| | | TCCGTCTGTGCTGGGCAGCAGACAGCTGCCAGGATCACGAACTCTGTAGTCAAAGA |
| | | AAAGAGTCGTGTGGCAGTTTCAGCTCTCGTTCATTGGGCAGCTCGCCTAGGCCCAG |
| | | CCTCTGAGCTGACATGGGAGTTGTTGGATTCTTTGTTTCATAGCTTTTTCTATGCC |
| | | ATAGGCAATATTGTTGTTCTTGGAAAGTTTATTATTTTTTTAACTCCCTTACTCTG |
| | | AGAAAGGGATATTTTGAAGGACTGTCATATATCTTTGAAAAAAGAAAATCTGTAAT |
| | | ACATATATTTTTATGTATGTTCACTGGCACTAAAAAATATAGAGAGCTTCATTCTG |
| | | TCCTTTGGGTAGTTGCTGAGGTAATTGTCCAGGTTGAAAAATAATGTGCTGATGCT |
| | | AGAGTCCCTCTCTGTCCATACTCTACTTCTAAATACATATAGGCATACATAGCAAG |
| | | TTTTATTTGACTTGTACTTTAAGAGAAAATATGTCCACCATCCACATGATGCACAA |
| | | ATGAGCTAACATTGAGCTTCAAGTAGCTTCTAAGTGTTTGTTTCATTAGGCACAGC |
| | | ACAGATGTGGCCTTTCCCCCCTTCTCTCCCTTGATATCTGGCAGGGCATAAAGGCC |
| | | CAGGCCACTTCCTCTGCCCCTTCCCAGCCCTGCACCAAAGCTGCATTTCAGGAGAC |
| | | TCTCTCCAGACAGCCCAGTAACTACCCGAGCATGGCCCCTGCATAGCCCTGGAAAA |
| | | ATAAGAGGCTGACTGTCTACGAATTATCTTGTGCCAGTTGCCCAGGTGAGAGGGCA |
| | | CTGGGCCAAGGGAGTGGTTTTCATGTTTGACCCACTACAAGGGGTCATGGGAATCA |
| | | GGAATGCCAAAGCACCAGATCAAATCCAAAACTTAAAGTCAAAATAAGCCATTCAG |
| | | CATGTTCAGTTTCTTGGAAAAGGAAGTTTCTACCCCTGATGCCTTTGTAGGCAGAT |
| | | CTGTTCTCACCATTAATCTTTTTGAAAATCTTTTAAAGCAGTTTTTAAAAAGAGAG |
| | | ATGAAAGCATCACATTATATAACCAAAGATTACATTGTACCTGCTAAGATACCAAA |
| | | ATTCATAAGGGCAGGGGGGGAGCAAGCATTAGTGCCTCTTTGATAAGCTGTCCAAA |
| | | GACAGACTAAAGGACTCTGCTGGTGACTGACTTATAAGAGCTTTGTGGGTTTTTTT |
| | | TTCCCTAATAATATACATGTTTAGAAGAATTGAAAATAATTTCGGGAAAATGGGAT |
| | | TATGGGTCCTTCACTAAGTGATTTTATAAGCAGAACTGGCTTTCCTTTTCTCTAGT |
| | | AGTTGCTGAGCAAATTGTTGAAGCTCCATCATTGCATGGTTGGAAATGGAGCTGTT |
| | | CTTAGCCACTGTGTTTGCTAGTGCCCATGTTAGCTTATCTGAAGATGTGAAACCCT |
| | | TGCTGATAAGGGAGCATTTAAAGTACTAGATTTTGCACTAGAGGGACAGCAGGCAG |
| | | AAATCCTTATTTCTGCCCACTTTGGATGGCACAAAAAGTTATCTGCAGTTGAAGGC |
| | | AGAAAGTTGAAATACATTGTAAATGAATATTTGTATCCATGTTTCAAAATTGAAAT |
| | | ATATATATATATATATATATATATATATATATATATATAGTGTGTGTGTGTGTTCT |
| | | GATAGCTTTAACTTTCTCTGCATCTTTATATTTGGTTCCAGATCACACCTGATGCC |
| | | ATGTACTTGTGAGAGAGGATGCAGTTTTGTTTTGGAAGCTCTCTCAGAACAAACAA |
| | | GACACCTGGATTGATCAGTTAACTAAAAGTTTTCTCCCCTATTGGGTTTGACCCAC |
| | | AGGTCCTGTGAAGGAGCAGAGGGATAAAAAGAGTAGAGGACATGATACATTGTACT |
| | | TTACTAGTTCAAGACAGATGAATGTGGAAAGCATAAAAACTCAATGGAACTGACTG |
| | | AGATTTACCACAGGGAAGGCCCAAACTTGGGGCCAAAAGCCTACCCAAGTGATTGA |
| | | CCAGTGGCCCCCTAATGGGACCTGAGCTGTTGGAAGAAGAGAACTGTTCCTTGGTC |
| | | TTCACCATCCTTGTGAGAGAAGGGCAGTTTCCTGCATTGGAACCTGGAGCAAGCGC |
| | | TCTATCTTTCACACAAATTCCCTCACCTGAGATTGAGGTGCTCTTGTTACTGGGTG |
| | | TCTGTGTGCTGTAATTCTGGTTTTGGATATGTTCTGTAAAGATTTTGACAAATGAA |
| | | AATGTGTTTTTCTCTGTTAAAACTTGTCAGAGTACTAGAAGTTGTATCTCTGTAGG |
| | | TGCAGGTCCATTTCTGCCCACAGGTAGGGTGTTTTTCTTTGATTAAGAGATTGACA |
| | | CTTCTGTTGCCTAGGACCTCCCAACTCAACCATTTCTAGGTGAAGGCAGAAAAATC |
| | | CACATTAGTTACTCCTCTTCAGACATTTCAGCTGAGATAACAAATCTTTTGGAATT |
| | | TTTTCACCCATAGAAAGAGTGGTAGATATTTGAATTTAGCAGGTGGAGTTTCATAG |
| | | TAAAAACAGCTTTTGACTCAGCTTTGATTTATCCTCATTTGATTTGGCCAGAAAGT |
| | | AGGTAATATGCATTGATTGGCTTCTGATTCCAATTCAGTATAGCAAGGTGCTAGGT |
| | | TTTTTCCTTTCCCCACCTGTCTCTTAGCCTGGGGAATTAAATGAGAAGCCTTAGAA |
| | | TGGGTGGCCCTTGTGACCTGAAACACTTCCCACATAAGCTACTTAACAAGATTGTC |
| | | ATGGAGCTGCAGATTCCATTGCCCACCAAAGACTAGAACACACACATATCCATACA |
| | | CCAAAGGAAAGACAATTCTGAAATGCTGTTTCTCTGGTGGTTCCCTCTCTGGCTGC |
| | | TGCCTCACAGTATGGGAACCTGTACTCTGCAGAGGTGACAGGCCAGATTTGCATTA |
| | | TCTCACAACCTTAGCCCTTGGTGCTAACTGTCCTACAGTGAAGTGCCTGGGGGGTT |
| | | GTCCTATCCCATAAGCCACTTGGATGCTGACAGCAGCCACCATCAGAATGACCCAC |
| | | GCAAAAAAAAGAAAAAAAAAATTAAAAAGTCCCCTCACAACCCAGTGACACCTTTC |
| | | TGCTTTCCTCTAGACTGGAACATTGATTAGGGAGTGCCTCAGACATGACATTCTTG |
| | | TGCTGTCCTTGGAATTAATCTGGCAGCAGGAGGGAGCAGACTATGTAAACAGAGAT |
| | | AAAAATTAATTTTCAATATTGAAGGAAAAAAGAAATAAGAAGAGAGAGAGAAAGAA |
| | | AGCATCACACAAAGATTTTCTTAAAAGAAACAATTTTGCTTGAAATCTCTTTAGAT |
| | | GGGGCTCATTTCTCACGGTGGCACTTGGCCTCCACTGGGCAGCAGGACCAGCTCCA |
| | | AGCGCTAGTGTTCTGTTCTCTTTTTGTAATCTTGGAATCTTTTGTTGCTCTAAATA |
| | | CAATTAAAAATGGCAGAAACTTGTTTGTTGGACTACATGTGTGACTTTGGGTCTGT |
| | | CTCTGCCTCTGCTTTCAGAAATGTCATCCATTGTGTAAAATATTGGCTTACTGGTC |
| | | TGCCAGCTAAAACTTGGCCACATCCCCTGTTATGGCTGCAGGATCGAGTTATTGTT |
| | | AACAAAGAGACCCAAGAAAAGCTGCTAATGTCCTCTTATCATTGTTGTTAATTTGT |
| | | TAAAACATAAAGAAATCTAAAATTTCA |
| 22 | COL9A2 | GAACAGCCAGCGCTGGAGGAGCGCCGGGAGACTCTGCCGTCGGTGCGTGCGCGGAC |
| | | ACGCACCCGTCCCCCTTGGTCTCGCCGCCAGCCATGGCCGCCGCTACGGCCTCCCC |
| | | CCGCAGCCTCCTTGTTCTCCTCCAGGTGGTAGTGCTCGCTCTGGCGCAGATTAGAG |
| | | GTCCACCGGGAGAGCGGGGCCCCCCGGGTCCCCCGGGACCGCCGGGAGTGCCTGGA |
| | | TCCGACGGCATCGACGGTGACAATGGGCCCCCTGGAAAAGCTGGCCCTCCGGGACC |
| | | CAAGGGCGAGCCTGGCAAAGCTGGGCCAGATGGGCCAGACGGGAAGCCCGGGATTG |
| | | ATGGTTTAACTGGAGCCAAGGGGGAGCCTGGCCCCATGGGGATCCCTGGAGTCAAG |
| | | GGCCAGCCCGGGCTTCCTGGTCCTCCTGGCCTTCCGGGCCCTGGTTTTGCTGGACC |
| | | TCCTGGGCCTCCTGGACCTGTTGGCCTCCCTGGTGAGATTGGAATCCGAGGCCCCA |
| | | AGGGGGACCCTGGACCAGATGGACCATCGGGGCCCCCAGGACCCCCTGGGAAACCT |
| | | GGTCGCCCGGGAACCATCCAGGGTCTGGAAGGCAGTGCGGATTTCCTGTGTCCAAC |
| | | CAACTGTCCACCCGGAATGAAAGGTCCCCCAGGGCTGCAGGGAGTGAAGGGGCATG |
| | | CGGGCAAACGCGGGATTCTGGGTGATCCTGGCCACCAGGGGAAGCCGGGTCCCAAG |
| | | GGAGATGTGGGTGCCTCTGGAGAGCAAGGCATCCCTGGACCACCGGGTCCCCAGGG |
| | | CATCAGGGGCTACCCAGGCATGGCAGGGCCCAAGGGAGAGACGGGCCCTCATGGAT |
| | | ATAAAGGCATGGTGGGCGCTATCGGTGCCACTGGGCCACCGGGTGAGGAAGGTCCT |
| | | AGGGGACCGCCAGGCCGAGCTGGGGAGAAGGGTGACGAGGGCAGCCCAGGTATTCG |
| | | TGGACCCCAGGGGATCACAGGCCCGAAAGGAGCAACGGGCCCCCCAGGCATCAACG |
| | | GCAAGGATGGGACCCCAGGCACGCCTGGCATGAAGGGCAGTGCAGGACAGGCGGGA |
| | | CAGCCCGGAAGTCCAGGCCACCAGGGCCTAGCGGGTGTGCCAGGCCAGCCTGGGAC |
| | | AAAAGGAGGCCCTGGAGACCAGGGTGAGCCGGGCCCGCAGGGCCTTCCTGGATTCT |
| | | CTGGTCCCCCTGGGAAAGAGGGAGAGCCAGGGCCTCGAGGAGAAATTGGTCCCCAG |
| | | GGCATCATGGGACAGAAGGGTGACCAAGGCGAGAGGGGTCCAGTGGGGCAACCAGG |
| | | CCCTCAGGGAAGGCAGGGCCCTAAGGGGGAGCAGGGCCCCCCCGGAATTCCAGGGC |
| | | CCCAAGGCTTGCCAGGCGTCAAAGGAGACAAGGGCTCCCCAGGGAAGACCGGGCCC |
| | | CGCGGCAAAGTGGGTGACCCAGGGGTGGCCGGCCTCCCCGGAGAGAAAGGCGAGAA |
| | | GGGCGAGTCCGGCGAGCCGGGGCCCAAGGGACAGCAAGGAGTACGTGGAGAACCCG |
| | | GCTACCCTGGCCCCAGCGGGGATGCGGGCGCCCCAGGGGTTCAGGGCTACCCTGGT |
| | | CCCCCCGGCCCTCGAGGACTGGCCGGGAACCGAGGCGTGCCAGGACAGCCCGGGAG |
| | | ACAGGGCGTGGAGGGCCGGGATGCCACTGACCAGCACATCGTGGATGTGGCGCTGA |
| | | AGATGCTGCAAGAGCAACTGGCAGAGGTCGCCGTGAGTGCCAAGCGGGAAGCCCTG |
| | | GGTGCGGTGGGCATGATGGGTCCTCCAGGACCTCCTGGGCCCCCTGGGTACCCAGG |
| | | CAAGCAGGGCCCCCATGGGCACCCTGGCCCTCGGGGCGTTCCTGGCATCGTGGGAG |
| | | CCGTGGGTCAGATCGGCAACACGGGGCCCAAGGGAAAACGTGGAGAGAAGGGTGAT |
| | | CCAGGAGAAGTGGGACGGGGGCACCCCGGGATGCCTGGGCCCCCAGGGATCCCAGG |
| | | ACTCCCTGGCCGGCCTGGCCAGGCAATCAACGGCAAGGATGGAGATCGAGGGTCCC |
| | | CAGGGGCTCCAGGAGAGGCAGGTCGACCTGGCCTGCCAGGCCCCGTGGGGCTGCCG |
| | | GGCTTCTGTGAACCTGCCGCCTGCCTTGGAGCTTCGGCCTATGCCTCTGCCCGCCT |
| | | TACAGAGCCTGGATCCATCAAGGGGCCTTGAGCATCAGGCCCAGACAGAGCCTGGC |
| | | AGGCATCCTGGCGGGAAGGACCAGGTCCCCTCTGGGTGGACATGCACCCATCCCCA |
| | | GTCCAGGAAACCATCTCCCCCAGGACCTTCTGTCTGGGACTCAGGAGTCCTAAGGA |
| | | AAAGGAATTCTAAAACATGGGGGAAGGGGAGGTAGAGCACTGATGGGTGAAAAAGT |
| | | GAGGCCAACACACAGGGCAAGTGGTGTCGATGGAGTCGAAGCGCTGAAGGAATAGG |
| | | GCGGCTTTCCTTCCAGCGAGCATCATTCGGCTGTTACCAAAACAAACATCTTAATC |
| | | TGCACCTTTCTCCACTGGCCATCTTGTCCTTGGGTCAGTGGGACATGGGCACCTCG |
| | | GGAGGCCCGGGCCCTGCCCAGCTACAGTTCCACCCCTCAGCTTGAGGACCAATGAC |
| | | TGAGGTCTATGCCAGTTCCTGATCCCATCTCACTCTCTGGACCTACCAGGTGACTG |
| | | CTGCTGGGTGACTCCCCTGAGGCGGCTATACCCTTAAGCCAGCCCCACTACTTCCT |
| | | TCCCTGCCTCCCAGCTCAGTATTTAAACATCATCTCCCTTCTCTTTCTCGCATAAC |
| | | TCCCCACCCCTTTCTCCCCGATCCACCCAGGCCTTTCTGTAAATAAAAGCTCCCAA |
| | | GTTGGGTACAAACCAGGATATTGGAGTTACTCTATCCTGGAGTTAACTAGGA |
| 23 | CRISP3 | GCACCTTCCTTCTGTCAATAGATGAAACAAATACTTCATCCTGCTCTGGAAACCAC |
| | | TGCAATGACATTATTCCCAGTGCTGTTGTTCCTGGTTGCTGGGCTGCTTCCATCTT |
| | | TTCCAGCAAATGAAGATAAGGATCCCGCTTTTACTGCTTTGTTAACCACCCAAACA |
| | | CAAGTGCAAAGGGAGATTGTGAATAAGCACAATGAACTGAGGAGAGCAGTATCTCC |
| | | CCCTGCCAGAAACATGCTGAAGATGGAATGGAACAAAGAGGCTGCAGCAAATGCCC |
| | | AAAAGTGGGCAAACCAGTGCAATTACAGACACAGTAACCCAAAGGATCGAATGACA |
| | | AGTCTAAAATGTGGTGAGAATCTCTACATGTCAAGTGCCTCCAGCTCATGGTCACA |
| | | AGCAATCCAAAGCTGGTTTGATGAGTACAATGATTTTGACTTTGGTGTAGGGCCAA |
| | | AGACTCCCAACGCAGTGGTTGGACATTATACACAGGTTGTTTGGTACTCTTCATAC |
| | | CTCGTTGGATGTGGAAATGCCTACTGTCCCAATCAAAAAGTTCTAAAATACTACTA |
| | | TGTTTGCCAATATTGTCCTGCTGGTAATTGGGCTAATAGACTATATGTCCCTTATG |
| | | AACAAGGAGCACCTTGTGCCAGTTGCCCAGATAACTGTGACGATGGACTATGCACC |
| | | AATGGTTGCAAGTACGAAGATCTCTATAGTAACTGTAAAAGTTTGAAGCTCACATT |
| | | AACCTGTAAACATCAGTTGGTCAGGGACAGTTGCAAGGCCTCCTGCAATTGTTCAA |
| | | ACAGCATTTATTAAATACGCATTACACACCGAGTAGGGCTATGTAGAGAGGAGTCA |
| | | GATTATCTACTTAGATTTGGCATCTACTTAGATTTAACATATACTAGCTGAGAAAT |
| | | TGTAGGCATGTTTGATACACATTTGATTTCAAATGTTTTTCTTCTGGATCTGCTTT |
| | | TTATTTTACAAAAATATTTTTCATACAAATGGTTAAAAAGAAACAAAATCTATAAC |
| | | AACAACTTTGGATTTTTATATATAAACTTTGTGATTTAAATTTACTGAATTTAATT |
| | | AGGGTGAAAATTTTGAAAGTTGTATTCTCATATGACTAAGTTCACTAAAACCCTGG |
| | | ATTGAAAGTGAAAATTATGTTCCTAGAACAAAATGTACAAAAAGAACAATATAATT |
| | | TTCACATGAACCCTTGGCTGTAGTTGCCTTTCCTAGCTCCACTCTAAGGCTAAGCA |
| | | TCTTCAAAGACGTTTTCCCATATGCTGTCTTAATTCTTTTCACTCATTCACCCTTC |
| | | TTCCCAATCATCTGGCTGGCATCCTCACAATTGAGTTGAAGCTGTTCCTCCTAAAA |
| | | CAATCCTGACTTTTATTTTGCCAAAATCAATACAATCCTTTGAATTTTTTATCTGC |
| | | ATAAATTTTACAGTAGAATATGATCAAACCTTCATTTTTAAACCTCTCTTCTCTTT |
| | | GACAAAACTTCCTTAAAAAAGAATACAAGATAATATAGGTAAATACCCTCCACTCA |
| | | AGGAGGTAGAACTCAGTCCTCTCCCTTGTGAGTCTTCACTAAAATCAGTGACTCAC |
| | | TTCCAAAGAGTGGAGTATGGAAAGGGAAACATAGTAACTTTACAGGGGAGAAAAAT |
| | | GACAAATGACGTCTTCACCAAGTGATCAAAATTAACGTCACCAGTGATAAGTCATT |
| | | CAGATTTGTTCTAGATAATCTTTCTAAAAATTCATAATCCCAATCTAATTATGAGC |
| | | TAAAACATCCAGCAAACTCAAGTTGAAGGACATTCTACAAAATATCCCTGGGGTAT |
| | | TTTAGAGTATTCCTCAAAACTGTAAAAATCATGGAAAATAAGGGAATCCTGAGAAA |
| | | CAATCACAGACCACATGAGACTAAGGAGACATGTGAGCCAAATGCAATGTGCTTCT |
| | | TGGATCAGATCCTGGAACAGAAAAAGATCAGTAATGAAAAAACTGATGAAGTCTGA |
| | | ATAGAATCTGGAGTATTTTTAACAGTAGTGTTGATTTCTTAATCTTGATAAATATA |
| | | GCAGGGTAATGTAAGATGATAACGTTAGAGAAACTGAAACTGGGTGAGGGCTATCT |
| | | AGGAATTCTCTGTACTATCTTACCAAATTTTCGGTAAGTCTAAGAAAGCAATGCAA |
| | | AATAAAAAGTGTCTTGAAAAAAAA |
| 24 | CST2 | GATCCCCGCCTCAGGCTCTCAACCTCCTCTCCTGCAGCTCCAGCTCTGTGCTCTGC |
| | | CTCCGAGGAGACCATGGCCTGGCCCCTGTGCACCCTGCTGCTCCTGCTGGCCACCC |
| | | AGGCTGTGGCCCTGGCCTGGAGCCCCCAGGAGGAGGACAGGATAATCGAGGGTGGC |
| | | ATCTATGATGCAGACCTCAATGATGAGCGGGTACAGCGTGCCCTTCACTTTGTCAT |
| | | CAGCGAGTATAACAAGGCCACTGAAGATGAGTACTACAGACGCCTGCTGCGGGTGC |
| | | TACGAGCCAGGGAGCAGATCGTGGGCGGGGTGAATTACTTCTTCGACATAGAGGTG |
| | | GGCCGAACCATATGTACCAAGTCCCAGCCCAACTTGGACACCTGTGCCTTCCATGA |
| | | ACAGCCAGAACTGCAGAAGAAACAGTTGTGCTCTTTCCAGATCTACGAAGTTCCCT |
| | | GGGAGGACAGAATGTCCCTGGTGAATTCCAGGTGTCAAGAAGCCTAGGGATCTGTG |
| | | CCAGGGAGTCACACTGACCACCTCCTACTCCCACCCCTTGTAGTGCTCCCACCCCT |
| | | GGACTGGTGGCCCCCACCCTGTGGGAGGTCTCCCCATGCACCTGCAGCAGGAGAAG |
| | | ACAGAGAAGGCTGCAGGAGGCCTTTGTTGCTCAGCAGGGGACTCTGCCCTCCCTCC |
| | | TTCCTTTTGCTTCTCATAGCCCTGGTACATGGTACACACACCCCCACCTCCTGCAA |
| | | TTAAACAGTAGCATCACC |
| 25 | DLX1 | AGCAGCATCATGCTTAGACTTTTCAAAGAGACAAACTCCATTTTCTTATGAATGGA |
| | | AAGTGAAAACCCCTGTTCCGCTTAAATTGGGTTCCTTCCTGTCCTGAGAAACATAG |
| | | AGACCCCCAAAAGGGAAGCAGAGGAGAGAAAGTCCCACACCCAGACCCCGCGAGAA |
| | | GAGATGACCATGACCACCATGCCAGAAAGTCTCAACAGCCCCGTGTCGGGCAAGGC |
| | | GGTGTTTATGGAGTTTGGGCCGCCCAACCAGCAAATGTCTCCTTCTCCCATGTCCC |
| | | ACGGGCACTACTCCATGCACTGTTTACACTCGGCGGGCCATTCGCAGCCCGACGGC |
| | | GCCTACAGCTCAGCCTCGTCCTTCTCCCGACCGCTGGGCTACCCCTACGTCAACTC |
| | | GGTCAGCAGCCACGCATCCAGCCCCTACATCAGTTCGGTGCAGTCCTACCCGGGCA |
| | | GCGCCAGCCTCGCCCAGAGCCGCCTGGAGGACCCAGGGGCGGACTCGGAGAAGAGC |
| | | ACGGTGGTGGAAGGCGGTGAAGTGCGCTTCAATGGCAAGGGAAAAAAGATCCGTAA |
| | | ACCCAGGACGATTTATTCCAGTTTGCAGTTGCAGGCTTTGAACCGGAGGTTCCAGC |
| | | AAACTCAGTACCTAGCTCTGCCGGAGAGGGCGGAGCTCGCGGCCTCTTTGGGACTC |
| | | ACACAGACTCAGGTCAAGATCTGGTTCCAAAACAAGCGATCCAAGTTCAAGAAGCT |
| | | GATGAAGCAGGGTGGGGCGGCTCTGGAGGGTAGTGCGTTGGCCAACGGTCGGGCCC |
| | | TGTCTGCTGGCTCCCCACCCGTGCCGCCCGGCTGGAACCCTAACTCTTCATCCGGG |
| | | AAGGGCTCAGGAGGAAACGCGGGCTCCTATATCCCCAGCTACACATCGTGGTACCC |
| | | TTCAGCGCACCAAGAAGCTATGCAGCAACCCCAACTTATGTGAGGTTGCCCGCCCG |
| | | TCTCCTTCTTGTCTCCCCGGCCCAGGTCCCTCCCGCCTCCAGGTCCATCCATCCCG |
| | | TCCGGAAAAGAAGGACCCAGAGGGAAGAAGGAACAGTGGAGGCGGGACGCCCTCCA |
| | | TCTCCTCGGAGCCCCGCGAGGTCCGGCCCAGCAACTTCCCGGCATCCGCGCTCTAG |
| | | CCTGAACCCTGGCCTGGGCCGAGCAGTGGCAGCAGAGAGTGGCCTCGGAGGGAAGC |
| | | CACTGCCACCTGAGACAGCCCAAGCAGCAAGATAAACCCGCTCCACCCGACCCGCC |
| | | GACCTTCAGCTTTGTGGGACTATCAGGAAAAAACAAAACAAAAACAAAATGTAGAA |
| | | AAAGCAAAAGCTCTTTTCTGTCCTGTCAGTCTCCTGTCTCCTTTTGCTCTGTCTGT |
| | | GCGCTGGTAAAGTCCAGGTCCTCATCCGTCCGCTGTCCTCATTCTGCGGCCTCAGC |
| | | AAAAAGCCACAAGGTCTGAGCGGCCCGGGTCCTGCCGGGCTGACCATCTCCGGATC |
| | | CTGGGACACTCTGCCTGACCATCTGTGTAGCTGGTGTGGGAATCTGGGGGCATTGG |
| | | AGGGAGGGGGTTTTATTTATTGAGAAATGGACTTCGCCTGAGGCTGTTTGCCAATT |
| | | CAGGGTTCTGCTGGGCGCAAGGAACGCACTGTTCAAACGCACTGTTTACTTTAAGC |
| | | GCACGGGGAGAAACGAATAAGGAGGACGTGGTGATTTTTAATTTATACAGTAACTT |
| | | TTGTACTTCTCTGGTATGGAGAGTTTGGAGCCGAATGATTTGCATTTTTTACATGT |
| | | CCGACATTATTTAATAAATAATTTTTAAAAGAAAAGAACGATAAATGAAGCCAACA |
| | | TGATTTTCTCATTTCGGGAGGAACTCTGTTGCTTCGCCTGGACAAGAAGGAAAATG |
| | | CTGATTTCCTCCTTGGGTAGAAAGAGGGAGCGAGGGCAAATGGGGAGTAGAGAGAA |
| | | AACAGGCGAGAACAAGCACTCTAATTCCAGTGGGCTTTAAAATAAGACAAAATCAG |
| | | CTTTACAACAATCCCTAGAGGCTCGACCACAGAATAATGCCAGTCACCACCCTGAA |
| | | CGCACAATCTCCAGTGCAGGATCTAATGACTGTACATATTATTGTTATTATTATTA |
| | | TTGTTATTATTGTTGTTCTGTAAACATGTTGCACAAGCTTAGCCTTTTTGCGTTCT |
| | | GTTGTGTGTGGCTGTAAAACCCCATGCTTTGTGAAATGAGAATCTTGACATTTTTC |
| | | TTGTGAAATTTGGAAAATGTGATCAATTGAAATCAACTGTGTTTTGTGTTCTCTAT |
| | | GTCAAAGTTTAGTTTTATATTGAGAATGTTAACTTATTGCTTTGTATCTTGGGAAA |
| | | AAAACTTTGTAAATAAGTTATAAAGTTTCTTTGAGACAGTAAAATTATGATTTCTT |
| | | GAAA |
| 26 | ETV1 | AGAGGCGCTTTCGGCTTCCAAGGGGGAAGTGCTGGGCTATAATTAATGTTTTTATT |
| | | AAATTTGGAGGGAAGTTTTTGCAGCCTTTCGCCTAGCGTGGCCTTCAGGTTGATAG |
| | | AAGTCCAGATCCTGAGGAAATCTCCAGCTAAATGCTCAAAATATAAAATACTGAGC |
| | | TGAGATTTGCGAAGAGCAGCAGCATGGATGGATTTTATGACCAGCAAGTGCCTTAC |
| | | ATGGTCACCAATAGTCAGCGTGGGAGAAATTGTAACGAGAAACCAACAAATGTCAG |
| | | GAAAAGAAAATTCATTAACAGAGATCTGGCTCATGATTCAGAAGAACTCTTTCAAG |
| | | ATCTAAGTCAATTACAGGAAACATGGCTTGCAGAAGCTCAGGTACCTGACAATGAT |
| | | GAGCAGTTTGTACCAGACTATCAGGCTGAAAGTTTGGCTTTTCATGGCCTGCCACT |
| | | GAAAATCAAGAAAGAACCCCACAGTCCATGTTCAGAAATCAGCTCTGCCTGCAGTC |
| | | AAGAACAGCCCTTTAAATTCAGCTATGGAGAAAAGTGCCTGTACAATGTCAGTGCC |
| | | TATGATCAGAAGCCACAAGTGGGAATGAGGCCCTCCAACCCCCCCACACCATCCAG |
| | | CACGCCAGTGTCCCCACTGCATCATGCATCTCCAAACTCAACTCATACACCGAAAC |
| | | CTGACCGGGCCTTCCCAGCTCACCTCCCTCCATCGCAGTCCATACCAGATAGCAGC |
| | | TACCCCATGGACCACAGATTTCGCCGCCAGCTTTCTGAACCCTGTAACTCCTTTCC |
| | | TCCTTTGCCGACGATGCCAAGGGAAGGACGTCCTATGTACCAACGCCAGATGTCTG |
| | | AGCCAAACATCCCCTTCCCACCACAAGGCTTTAAGCAGGAGTACCACGACCCAGTG |
| | | TATGAACACAACACCATGGTTGGCAGTGCGGCCAGCCAAAGCTTTCCCCCTCCTCT |
| | | GATGATTAAACAGGAACCCAGAGATTTTGCATATGACTCAGGCTGTATGTTTGAAA |
| | | AGGGCCCCAGGCAGTTTTATGATGACACCTGTGTTGTCCCAGAAAAATTCGATGGA |
| | | GACATCAAACAAGAGCCAGGAATGTATCGGGAAGGACCCACATACCAACGGCGAGG |
| | | ATCACTTCAGCTCTGGCAGTTTTTGGTAGCTCTTCTGGATGACCCTTCAAATTCTC |
| | | ATTTTATTGCCTGGACTGGTCGAGGCATGGAATTTAAACTGATTGAGCCTGAAGAG |
| | | GTGGCCCGACGTTGGGGCATTCAGAAAAACAGGCCAGCTATGAACTATGATAAACT |
| | | TAGCCGTTCACTCCGCTATTACTATGAGAAAGGAATTATGCAAAAGGTGGCTGGAG |
| | | AGAGATATGTCTACAAGTTTGTGTGTGATCCAGAAGCCCTTTTCTCCATGGCCTTT |
| | | CCAGATAATCAGCGTCCACTGCTGAAGACAGACATGGAACGTCACATCAACGAGGA |
| | | GGACACAGTGCCTCTTTCTCACTTTGATGAGAGCATGGCCTACATGCCGGAAGGGG |
| | | GCTGCTGCAACCCCCACCCCTACAACGAAGGCTACGTGTATTAACACAAGTGACAG |
| | | TCAAGCAGGGCGTTTTTGCGCTTTTCCTTTTTTCTGCAAGATACAGAGAATTGCTG |
| | | AATCTTTGTTTTATTTCTGTTGTTTGTATTTTATTTTTAAATAATAATACACAAAA |
| | | AGGGGCTTTTCCTGTTGCATTATTCTATGGTCTGCCATGGACTGTGCACTTTATTT |
| | | GAGGGTGGGTGGGAGTAATCTAAACATTTATTCTGTGTAACAGGAAGCTAATGGGT |
| | | GAATGGGCAGAGGGATTTGGGGATTACTTTTTACTTAGGCTTGGGATGGGGTCCTA |
| | | CAAGTTTTGAGTATGATGAAACTATATCATGTCTGTTTGATTTCATAACAACATAA |
| | | GATAATGTTTATTTTATCGGGGTATCTATGGTACAGTTAATTTCACGTTGTGTAAA |
| | | TATCCACTTGGAGACTATTTGCCTTGGGCATTTTCCCCTGTCATTTATGAGTCTCT |
| | | GCAGGTGTACAAAAAAACCCCAATCTACTGTAAATGGCAGTTTAATTGTTAGAAAT |
| | | GACTGTTTTTGCACCACTTGTAAAAAGGTATTTAGCGATTGCATTTGCTGTTTGTT |
| | | GTTTTATTTTGCTTTATATATGACTTGCAGAGGATAACCATAAAATGGGTAATTCT |
| | | CTCTGAAGTTGAATAATCACCATGACTGTAAATGAGGGGCACAATTTTGGACTCTG |
| | | GCGCCAAACTGAGTCATAGGCCAGTAGCATTACGTGTATCTGGTGCCACCTTGCTG |
| | | TTTAGATACAAATCATACCGTCTTTTAAATATTTTGAAGCCCATTTCAGTTAAATA |
| | | ATGACATGTCATGGTCCTTTGGAATCTTCATTTAAATGTTAAATCTGGAATCAAAA |
| | | TGAAGCAAAAAATATCTGTCTCCTTTTCACTTTCTTCAGTACATAAATACATTATT |
| | | TAATCAATAAGAATTAACTGTACTAAATCATGTATTATGCTGTTCTAGTTACAGCA |
| | | AACACTCTTTAAGAAAAATATCCAATACACTAAATAGGTACTATAGTAATTTTTAG |
| | | ACATGGTACCCATTGATATGCATTTAAACCTTTTACTGCTGTGTTATGTTGATAAC |
| | | ATATATAAATATTAGATAATGCTAATGCTTCTGCTGCTGTCTTTTCTGTAATATTC |
| | | TCTTTCATGCTGAATTTACTATGACCATTTATAAGCAGTGCAGTTAACTACAGATA |
| | | GCATTTCAGGACAAAATAGATGACTCAAACCATTTATTGCTTAAAAAATAGCTTAC |
| | | GCCATGCTATGCTATAAGCAGCTTTTATGCACATTGACAAATGAAGAGTAAGCTTC |
| | | AGCTTGCTAAAGGAAACTGTGGAACCTTTTGTAACTTTTGGTGATATGGAAAATTA |
| | | TTTACAAACCGTCAAAGAATATGAGGAAGTTGCTGTATGACATAGTGCTGGCACTG |
| | | ATATTATCCATCATCTCTTTTTGGACACTTCTGTAAATGTGATTGGATTGTTTGAA |
| | | AGAAGATTTAAAGTTTCAAAGTTTTTTGTTCTGTTTTTGCTTTGCATTTGGAGAAA |
| | | ATATTGAAAGCAGGGTATGTTGTTTCATTCACCTTGAAAAAACCATGAGTAAATGG |
| | | GGATATAGAATCTCTGAATAGCTCGCTAAAAGATTCAAGCAAGGGACATGAATTTT |
| | | GTTCCATCTATCAATAATATCCAGAAGAACAACTTTTTTAAAGAGTCTATAGCAAA |
| | | AAGCAAAAAAAAAAAAAAATTCTAAACACAAAGTCAAAATAAACCTATTGTAAAAG |
| | | CATTTCGTGATGAGCATGAAAAAGATTGTTTAAAGATGATCCCCCCAGCTACCCAT |
| | | TTTCCAAAACTACACAGATCACAGCTCATTTCTCTAAGTGGAGCAGTTATCAAGAA |
| | | ACCCAAACACCAAAATTGCTACTCTTCACATTTAATCCTACAAAAAGTACTCCAAT |
| | | TTCAAAATATGTATGTAACCTGCGATTTCAATGATTGTTGTTCATATACATCATGT |
| | | ATTATTTTGGCCCATTTTGGGCCTAAAAAAGAAAACTATGCCTTAAAAATCAGAAC |
| | | CTTTTCTCCCCACTATGCTTATGTGGCCATCTACAGCACTTAGAATAAAAACAGAT |
| | | GTTAAAATATTCAGTGAAAGTTTTATTGGAAAAAGGAATTGAGATATATAATTGAG |
| | | ATTTGGTGAAATTGAAGGAGAAAATTTAAGTGAGTCTTTAAAATATATTCTGAATG |
| | | AAAACTGTATTGAGGATTCATTTTTGTTCCTTTTTTTTCTTTTTCTCTTTTCTCCT |
| | | TTTTCTTCTTTTTAATAGTCTAGTTTTAGTCAGTCAGTGAGGAAGAATTGGGCCAT |
| | | GCTAACGTTATCACAAGAGAACAATGGCAGAAATGGTATTAGTTATATAATATTTA |
| | | AGGACAAACTATATGTTTTGCTGTTTTAACGTAGTGACTCACTGAACTAAATACAT |
| | | AATTGACCAACATTAAGTGTATTTCCAATACAGAAGGGTTGAAAATATTACATTAT |
| | | AAACTCTTTTGAAAAATGTATCTAAAATTTTTTAAGTTCTGTTTTGATTCCACTTT |
| | | TTGGTTGAGTTTTTATGTTTTTGTTTTCAGGTAGATTAATAAATCTGGCAGCTGAT |
| | | TTCTGCAAGATTCTTGTGTTTTGAATTTCTCATTGAATTGGCTACTCAAACATAGA |
| | | AATCATTTGTTAATGATGTAATGTCTTCTCTCAGCTTTTATCTTCACTGCTGTTTG |
| | | CTGTCTCTTGATGATGACATGTTAATACCCAATAGATTAATTGCAACAAACACTTA |
| | | TACTCAAATAACTAAGTAAAAATAATTTTTCTTGTTATGTCCATGAAAAGTGCTTC |
| | | AGAATAAAAATCCACAAGACTGACAGTGCAGAACATTTTTCTCAAATCATGGGCGG |
| | | ATCTTGGAGGTCTAGTTTCCCGTAGATGCTGTAACCAATTACCACAACTTCAGTAA |
| | | TTTACACAAATTTATCTTATAGTTCTGGAGGCAGAAGTTCAAAAGAAGCCTTAAGA |
| | | GACTAAAACCAAGATGTCCTTAGGTCTGGTTCCTTCTGGAGGCTCCAGGGGAGATT |
| | | CTTCCAGCTTTCACTTCTAGAGTCTGCTGACATTCCTTGGCTCCTGGCTACATCAC |
| | | TTCAATCTCTGCTTCCATGGTCACATACTCTTCTACTATAGTCAAATTTCCTTCCT |
| | | GCCTCTTATAAGGATGCTTGTGATTACATTTAGGGGATGCTCAGATAATCCAGGAC |
| | | AATCTCTCCATCTCAAGATCCTTAACTTAATGACGTGTGCCAAGTCCCTTTGGCTA |
| | | GATAATTATTCATAGGTCCCAGGGATTAGGACATGGATGTAAGGGGTGAGGGCAGG |
| | | GCTGTTATTCAGAACACCGCACGGAGGAGGAAGACTGTGTAGCAAAGACTCTAATT |
| | | GATTTACTCAGGAACAGTGGAGTTCTGCTGAGGGATCTAGGATTTGAAAGTACTAG |
| | | AGTTTGCTTTTATTTACCACTGAGATATTTTCCCCTTATTCTGCATAAATAATTTT |
| | | GAAAACTTTCTATATTAAATTTCAACTATTCCACTAAAATGTCTGGTAATCACATC |
| | | AAGCCTTTAGATTATTCAAATCCTTCCCCAGCCCCCAGGAAAACACTAAGTCATGA |
| | | AACAGAAAAACAGAAGGTATGATAATAATAGTAATAACAGTTAAATCAGTGGTCTA |
| | | ATCCAGATTTTATTTTTTAATACATTTCTTTTGGTGTTAATATGGGTTACTATGTG |
| | | ATCTTATCATTTGCTAGTGATTATTACTTATTAGGTAAGAACAATGTGTAAAATAT |
| | | GTCTATTACTCAAAAGAACAATTGCAAAATGAGTCAACTTATCTTTATATAACCAG |
| | | GAAAGAAATATATTGCCAGAAGCTACAGAATTTTGCCAGATGATAGGGATTTCTAA |
| | | AATGAGCCACTTTGTCTATCATGCAGCCTTTTCAGAGCTTGTAATGAGAAAACATT |
| | | ACAGAGGAGAAGGTCATTTGGATGTTTGTTACTTGGAATCCTAGAAAACAAAAACT |
| | | AAAATTTAAAAATAAGAAGTGAGTAAGCTATTTTCCATTTGCGATTTGGTATGGAG |
| | | AAGAGAGGAAATAGAATTATTAAAAAAATACAAATTGGGTAAAAGTGATGGTGGAA |
| | | AAAATATAAAGAAGGCAAATGTACATATTAAGCAATTCTACTAAGAATTGGAAAAA |
| | | TCAAGTTTCAAAAAGATGGTAATAGTTGGGCATGATACTAGAAAATTTCACCCAGT |
| | | TTATTCAGAGCTCAACTAGTACTTTTAGGACTTCTTTTTTTATATACATGAGACTC |
| | | ACTTTGACATACTTAAAAAAAAAACAGTTTATGGAAAGTACAGTTTAAGAGGAGAA |
| | | TTTGATTAGACTAAGTGGATATCTTTATAGAAATATTAATGATTTCAGAATTTTCA |
| | | GTTACAAGTGTATATACCGTGGCTATTGTTTATGGATTCATATGTAAGGTAGGGTC |
| | | TTTTTTGCATATAGACTCCAGTATTAGTTACTTTCATTCTAAAATTATATTTATGC |
| | | TTCTATGGGGAAGAAAATTTTTAATTCACTTGGTTGTATTAAAATTATACTTACGG |
| | | TTTGAGAAAACATGCTATGAAAATCATGATTATAGCAAATTAAATATGCTCAAAAT |
| | | TTAAATCTAAAATAAAAGCCCAGAAACTGAAAA |
| 27 | F5 | ATTGCAGCTGGGACAGCCCGGAGTGTGGTTAGCAGCTCGGCAAGCGCTGCCCAGGT |
| | | CCTGGGGTGGTGGCAGCCAGCGGGAGCAGGAAAGGAAGCATGTTCCCAGGCTGCCC |
| | | ACGCCTCTGGGTCCTGGTGGTCTTGGGCACCAGCTGGGTAGGCTGGGGGAGCCAAG |
| | | GGACAGAAGCGGCACAGCTAAGGCAGTTCTACGTGGCTGCTCAGGGCATCAGTTGG |
| | | AGCTACCGACCTGAGCCCACAAACTCAAGTTTGAATCTTTCTGTAACTTCCTTTAA |
| | | GAAAATTGTCTACAGAGAGTATGAACCATATTTTAAGAAAGAAAAACCACAATCTA |
| | | CCATTTCAGGACTTCTTGGGCCTACTTTATATGCTGAAGTCGGAGACATCATAAAA |
| | | GTTCACTTTAAAAATAAGGCAGATAAGCCCTTGAGCATCCATCCTCAAGGAATTAG |
| | | GTACAGTAAATTATCAGAAGGTGCTTCTTACCTTGACCACAC |
| | | ATTCCCTGCGGAGAAGATGGACGACGCTGTGGCTCCAGGCCGAGAATACACCTATG |
| | | AATGGAGTATCAGTGAGGACAGTGGACCCACCCATGATGACCCTCCATGCCTCACA |
| | | CACATCTATTACTCCCATGAAAATCTGATCGAGGATTTCAACTCGGGGCTGATTGG |
| | | GCCCCTGCTTATCTGTAAAAAAGGGACCCTAACTGAGGGTGGGACACAGAAGACGT |
| | | TTGACAAGCAAATCGTGCTACTATTTGCTGTGTTTGATGAAAGCAAGAGCTGGAGC |
| | | CAGTCATCATCCCTAATGTACACAGTCAATGGATATGTGAATGGGACAATGCCAGA |
| | | TATAACAGTTTGTGCCCATGACCACATCAGCTGGCATCTGCTGGGAATGAGCTCGG |
| | | GGCCAGAATTATTCTCCATTCATTTCAACGGCCAGGTCCTGGAGCAGAACCATCAT |
| | | AAGGTCTCAGCCATCACCCTTGTCAGTGCTACATCCACTACC |
| | | GCAAATATGACTGTGGGCCCAGAGGGAAAGTGGATCATATCTTCTCTCACCCCAAA |
| | | ACATTTGCAAGCTGGGATGCAGGCTTACATTGACATTAAAAACTGCCCAAAGAAAA |
| | | CCAGGAATCTTAAGAAAATAACTCGTGAGCAGAGGCGGCACATGAAGAGGTGGGAA |
| | | TACTTCATTGCTGCAGAGGAAGTCATTTGGGACTATGCACCTGTAATACCAGCGAA |
| | | TATGGACAAAAAATACAGGTCTCAGCATTTGGATAATTTCTCAAACCAAATTGGAA |
| | | AACATTATAAGAAAGTTATGTACACACAGTACGAAGATGAGTCCTTCACCAAACAT |
| | | ACAGTGAATCCCAATATGAAAGAAGATGGGATTTTGGGTCCTATTATCAGAGCCCA |
| | | GGTCAGAGACACACTCAAAATCGTGTTCAAAAATATGGCCAGCCGCCCCTATAGCA |
| | | TTTACCCTCATGGAGTGACCTTCTCGCCTTATGAAGATGAAGTCAACTCTTCTTTC |
| | | ACCTCAGGCAGGAACAACACCATGATCAGAGCAGTTCAACCAGGGGAAACCTATAC |
| | | TTATAAGTGGAACATCTTAGAGTTTGATGAACCCACAGAAAATGATGCCCAGTGCT |
| | | TAACAAGACCATACTACAGTGACGTGGACATCATGAGAGACATCGCCTCTGGGCTA |
| | | ATAGGACTACTTCTAATCTGTAAGAGCAGATCCCTGGACAGGCGAGGAATACAGAG |
| | | GGCAGCAGACATCGAACAGCAGGCTGTGTTTGCTGTGTTTGATGAGAACAAAAGCT |
| | | GGTACCTTGAGGACAACATCAACAAGTTTTGTGAAAATCCTGATGAGGTGAAACGT |
| | | GATGACCCCAAGTTTTATGAATCAAACATCATGAGCACTATCAATGGCTATGTGCC |
| | | TGAGAGCATAACTACTCTTGGATTCTGCTTTGATGACACTGTCCAGTGGCACTTCT |
| | | GTAGTGTGGGGACCCAGAATGAAATTTTGACCATCCACTTCACTGGGCACTCATTC |
| | | ATCTATGGAAAGAGGCATGAGGACACCTTGACCCTCTTCCCCATGCGTGGAGAATC |
| | | TGTGACGGTCACAATGGATAATGTTGGAACTTGGATGTTAACTTCCATGAATTCTA |
| | | GTCCAAGAAGCAAAAAGCTGAGGCTGAAATTCAGGGATGTTAAATGTATCCCAGAT |
| | | GATGATGAAGACTCATATGAGATTTTTGAACCTCCAGAATCTACAGTCATGGCTAC |
| | | ACGGAAAATGCATGATCGTTTAGAACCTGAAGATGAAGAGAGTGATGCTGACTATG |
| | | ATTACCAGAACAGACTGGCTGCAGCATTAGGAATCAGGTCATTCCGAAACTCATCA |
| | | TTGAATCAGGAAGAAGAAGAGTTCAATCTTACTGCCCTAGCTCTGGAGAATGGCAC |
| | | TGAATTCGTTTCTTCAAACACAGATATAATTGTTGGTTCAAATTATTCTTCCCCAA |
| | | GTAATATTAGTAAGTTCACTGTCAATAACCTTGCAGAACCTCAGAAAGCCCCTTCT |
| | | CACCAACAAGCCACCACAGCTGGTTCCCCACTGAGACACCTCATTGGCAAGAACTC |
| | | AGTTCTCAATTCTTCCACAGCAGAGCATTCCAGCCCATATTCTGAAGACCCTATAG |
| | | AGGATCCTCTACAGCCAGATGTCACAGGGATACGTCTACTTTCACTTGGTGCTGGA |
| | | GAATTCAAAAGTCAAGAACATGCTAAGCATAAGGGACCCAAGGTAGAAAGAGATCA |
| | | AGCAGCAAAGCACAGGTTCTCCTGGATGAAATTACTAGCACATAAAGTTGGGAGAC |
| | | ACCTAAGCCAAGACACTGGTTCTCCTTCCGGAATGAGGCCCTGGGAGGACCTTCCT |
| | | AGCCAAGACACTGGTTCTCCTTCCAGAATGAGGCCCTGGAAGGACCCTCCTAGTGA |
| | | TCTGTTACTCTTAAAACAAAGTAACTCATCTAAGATTTTGGTTGGGAGATGGCATT |
| | | TGGCTTCTGAGAAAGGTAGCTATGAAATAATCCAAGATACTGATGAAGACACAGCT |
| | | GTTAACAATTGGCTGATCAGCCCCCAGAATGCCTCACGTGCTTGGGGAGAAAGCAC |
| | | CCCTCTTGCCAACAAGCCTGGAAAGCAGAGTGGCCACCCAAAGTTTCCTAGAGTTA |
| | | GACATAAATCTCTACAAGTAAGACAGGATGGAGGAAAGAGTAGACTGAAGAAAAGC |
| | | CAGTTTCTCATTAAGACACGAAAAAAGAAAAAAGAGAAGCACACACACCATGCTCC |
| | | TTTATCTCCGAGGACCTTTCACCCTCTAAGAAGTGAAGCCTACAACACATTTTCAG |
| | | AAAGAAGACTTAAGCATTCGTTGGTGCTTCATAAATCCAATGAAACATCTCTTCCC |
| | | ACAGACCTCAATCAGACATTGCCCTCTATGGATTTTGGCTGGATAGCCTCACTTCC |
| | | TGACCATAATCAGAATTCCTCAAATGACACTGGTCAGGCAAGCTGTCCTCCAGGTC |
| | | TTTATCAGACAGTGCCCCCAGAGGAACACTATCAAACATTCCCCATTCAAGACCCT |
| | | GATCAAATGCACTCTACTTCAGACCCCAGTCACAGATCCTCTTCTCCAGAGCTCAG |
| | | TGAAATGCTTGAGTATGACCGAAGTCACAAGTCCTTCCCCACAGATATAAGTCAAA |
| | | TGTCCCCTTCCTCAGAACATGAAGTCTGGCAGACAGTCATCTCTCCAGACCTCAGC |
| | | CAGGTGACCCTCTCTCCAGAACTCAGCCAGACAAACCTCTCTCCAGACCTCAGCCA |
| | | CACGACTCTCTCTCCAGAACTCATTCAGAGAAACCTTTCCCCAGCCCTCGGTCAGA |
| | | TGCCCATTTCTCCAGACCTCAGCCATACAACCCTTTCTCCAGACCTCAGCCATACA |
| | | ACCCTTTCTTTAGACCTCAGCCAGACAAACCTCTCTCCAGAACTCAGTCAGACAAA |
| | | CCTTTCTCCAGCCCTCGGTCAGATGCCCCTTTCTCCAGACCTCAGCCATACAACCC |
| | | TTTCTCTAGACTTCAGCCAGACAAACCTCTCTCCAGAACTCAGCCATATGACTCTC |
| | | TCTCCAGAACTCAGTCAGACAAACCTTTCCCCAGCCCTCGGTCAGATGCCCATTTC |
| | | TCCAGACCTCAGCCATACAACCCTTTCTCTAGACTTCAGCCAGACAAACCTCTCTC |
| | | CAGAACTCAGTCAAACAAACCTTTCCCCAGCCCTCGGTCAGATGCCCCTTTCTCCA |
| | | GACCCCAGCCATACAACCCTTTCTCTAGACCTCAGCCAGACAAACCTCTCTCCAGA |
| | | ACTCAGTCAGACAAACCTTTCCCCAGACCTCAGTGAGATGCCCCTCTTTGCAGATC |
| | | TCAGTCAAATTCCCCTTACCCCAGACCTCGACCAGATGACACTTTCTCCAGACCTT |
| | | GGTGAGACAGATCTTTCCCCAAACTTTGGTCAGATGTCCCTTTCCCCAGACCTCAG |
| | | CCAGGTGACTCTCTCTCCAGACATCAGTGACACCACCCTTCTCCCGGATCTCAGCC |
| | | AGATATCACCTCCTCCAGACCTTGATCAGATATTCTACCCTTCTGAATCTAGTCAG |
| | | TCATTGCTTCTTCAAGAATTTAATGAGTCTTTTCCTTATCCAGACCTTGGTCAGAT |
| | | GCCATCTCCTTCATCTCCTACTCTCAATGATACTTTTCTATCAAAGGAATTTAATC |
| | | CACTGGTTATAGTGGGCCTCAGTAAAGATGGTACAGATTACATTGAGATCATTCCA |
| | | AAGGAAGAGGTCCAGAGCAGTGAAGATGACTATGCTGAAATTGATTATGTGCCCTA |
| | | TGATGACCCCTACAAAACTGATGTTAGGACAAACATCAACTCCTCCAGAGATCCTG |
| | | ACAACATTGCAGCATGGTACCTCCGCAGCAACAATGGAAACAGAAGAAATTATTAC |
| | | ATTGCTGCTGAAGAAATATCCTGGGATTATTCAGAATTTGTACAAAGGGAAACAGA |
| | | TATTGAAGACTCTGATGATATTCCAGAAGATACCACATATAAGAAAGTAGTTTTTC |
| | | GAAAGTACCTCGACAGCACTTTTACCAAACGTGATCCTCGAGGGGAGTATGAAGAG |
| | | CATCTCGGAATTCTTGGTCCTATTATCAGAGCTGAAGTGGATGATGTTATCCAAGT |
| | | TCGTTTTAAAAATTTAGCATCCAGACCGTATTCTCTACATGCCCATGGACTTTCCT |
| | | ATGAAAAATCATCAGAGGGAAAGACTTATGAAGATGACTCTCCTGAATGGTTTAAG |
| | | GAAGATAATGCTGTTCAGCCAAATAGCAGTTATACCTACGTATGGCATGCCACTGA |
| | | GCGATCAGGGCCAGAAAGTCCTGGCTCTGCCTGTCGGGCTTGGGCCTACTACTCAG |
| | | CTGTGAACCCAGAAAAAGATATTCACTCAGGCTTGATAGGTCCCCTCCTAATCTGC |
| | | CAAAAAGGAATACTACATAAGGACAGCAACATGCCTATGGACATGAGAGAATTTGT |
| | | CTTACTATTTATGACCTTTGATGAAAAGAAGAGCTGGTACTATGAAAAGAAGTCCC |
| | | GAAGTTCTTGGAGACTCACATCCTCAGAAATGAAAAAATCCCATGAGTTTCACGCC |
| | | ATTAATGGGATGATCTACAGCTTGCCTGGCCTGAAAATGTATGAGCAAGAGTGGGT |
| | | GAGGTTACACCTGCTGAACATAGGCGGCTCCCAAGACATTCACGTGGTTCACTTTC |
| | | ACGGCCAGACCTTGCTGGAAAATGGCAATAAACAGCACCAGTTAGGGGTCTGGCCC |
| | | CTTCTGCCTGGTTCATTTAAAACTCTTGAAATGAAGGCATCAAAACCTGGCTGGTG |
| | | GCTCCTAAACACAGAGGTTGGAGAAAACCAGAGAGCAGGGATGCAAACGCCATTTC |
| | | TTATCATGGACAGAGACTGTAGGATGCCAATGGGACTAAGCACTGGTATCATATCT |
| | | GATTCACAGATCAAGGCTTCAGAGTTTCTGGGTTACTGGGAGCCCAGATTAGCAAG |
| | | ATTAAACAATGGTGGATCTTATAATGCTTGGAGTGTAGAAAAACTTGCAGCAGAAT |
| | | TTGCCTCTAAACCTTGGATCCAGGTGGACATGCAAAAGGAAGTCATAATCACAGGG |
| | | ATCCAGACCCAAGGTGCCAAACACTACCTGAAGTCCTGCTATACCACAGAGTTCTA |
| | | TGTAGCTTACAGTTCCAACCAGATCAACTGGCAGATCTTCAAAGGGAACAGCACAA |
| | | GGAATGTGATGTATTTTAATGGCAATTCAGATGCCTCTACAATAAAAGAGAATCAG |
| | | TTTGACCCACCTATTGTGGCTAGATATATTAGGATCTCTCCAACTCGAGCCTATAA |
| | | CAGACCTACCCTTCGATTGGAACTGCAAGGTTGTGAGGTAAATGGATGTTCCACAC |
| | | CCCTGGGTATGGAAAATGGAAAGATAGAAAACAAGCAAATCACAGCTTCTTCGTTT |
| | | AAGAAATCTTGGTGGGGAGATTACTGGGAACCCTTCCGTGCCCGTCTGAATGCCCA |
| | | GGGACGTGTGAATGCCTGGCAAGCCAAGGCAAACAACAATAAGCAGTGGCTAGAAA |
| | | TTGATCTACTCAAGATCAAGAAGATAACGGCAATTATAACACAGGGCTGCAAGTCT |
| | | CTGTCCTCTGAAATGTATGTAAAGAGCTATACCATCCACTACAGTGAGCAGGGAGT |
| | | GGAATGGAAACCATACAGGCTGAAATCCTCCATGGTGGACAAGATTTTTGAAGGAA |
| | | ATACTAATACCAAAGGACATGTGAAGAACTTTTTCAACCCCCCAATCATTTCCAGG |
| | | TTTATCCGTGTCATTCCTAAAACATGGAATCAAAGTATTGCACTTCGCCTGGAACT |
| | | CTTTGGCTGTGATATTTACTAGAATTGAACATTCAAAAACCCCTGGAAGAGACTCT |
| | | TTAAGACCTCAAACCATTTAGAATGGGCAATGTATTTTACGCTGTGTTAAATGTTA |
| | | ACAGTTTTCCACTATTTCTCTTTCTTTTCTATTAGTGAATAAAATTTTATACAAGA |
| | | AGCTTTTATAATGTAACTCCTTGCTACCAGTAAGTAAGATAATGGCTATTACTTCT |
| | | GCATTAATTTGAATACAGGTAGGAAAATATCAAGAACCAACAAGAAAAGGGCTTAT |
| | | CTTTCTTAATGATTGAAAATGCTATGAAGTAATATTTATGTAGTTAAAATGCTTCA |
| | | TTATAACTCTTTTAAATCCTTTACACACTAGTAAAACAGATATTACTTTAAATAAT |
| | | AATTGATAGACCTGGATAACTTTCACAAACACATGATTTTTTAATGGTTTTTCTTG |
| | | AGTGAAGAGAAAAACAATATTATCAAATGAAATAAGTACTTAAAATATCCTGTCTT |
| | | TCCCATATAACAATGATTTTTCTGACTTTCCATGAGTAAAAAAACAGCCAAGCATC |
| | | TTTCCAGTAGCCCCATTGAAATTGTGAATCCGTCCTGGTCTCCCTAAGGACTGCAC |
| | | ACATTGATATTCAAGGTTGGTGGTCATTAGATATGGAACAGAACTGAAATAACCAT |
| | | GGTAGAACTGAATGTGTAATGTTGGCTTTATTCTAGCTGGTACTACATGGCACACA |
| | | GTTTCAAAACATAATTTCACCTACTGGAAAGCTCAGACCTGTAAAACAGAGCATGG |
| | | GAACTGCTGGTCTAAATGCAGTTGTTCCTGCTCAAAGAGACCTCTGGCCAAACTGG |
| | | CAAGCAGTTAAAGTTTTCTTTCAGGGCCTTCCTCTCTATGGCCTCAACTTCCTCCT |
| | | CTCTCTTCTTCCAGCAACTTCCCCTTTCATCATTCCTTTCCCTGGGGACTTGGCAT |
| | | TCAGTGATCCTGTAGATATTGCACAACTGGGGAACCTTTAGACATCCTTAAAATCA |
| | | CATGAGATAGACAGTCATTTGGGGTGTCTGAAATAAACCACCCCAAAACTTAGTGT |
| | | TAAAAGAGCAACCAAAAAAAATTTATGTGAGATTATGGATTTGTTACTTAGCTTGA |
| | | TTTAATCATCCTGTAACGTGTACATATATCAAAATGTTATGTATACCATAAATATA |
| | | TAAAATTTTATCAACGAAATTCATAACAATCTCTCAGACCACAGAGAAATCAAATT |
| | | AGAACTGAGGACTAAGAAACTCACTCGAAACCACACAACTACATGGAAACTGAACA |
| | | ACCTGCTCCTGAATGACTACTGGGTAAATAATGAAATTAAGGCAGAAATAAATAAG |
| | | TTCCTTAAAACCAATGAGAACAAAGAGACAACATACCAGAATCTCTAGGAGACAGG |
| | | GCTTTGCTTTTGCTGCATTCTATTCGTTGTGAACACAAATTACAGGCCAGTCTCGA |
| | | TTCAGTGTAGAAGGGAACTGCATAAGGACCACATACCAGGAGGCATAATTCACTGG |
| | | GAGCATCTTTAGAAACTACCAGAGTTACCTGTTGCCCATACCAGTGGGGTAAGCCC |
| | | TATGAATGTATATGAGAGTTTCAAACATCCACAAAACATTGGCTTTCTAATATTCG |
| | | TATTCCCACTATTCCTTTCTTTTCATGATTCATGTCATTGTCCCATCAACATTTCT |
| | | AAGATTTCCATTCCGTTAAGAGCAAAAGAGAATGTTGGAAGGTGGGGGAAAACATT |
| | | TCTTTGTTTTCTACAGGGCCAGCTTCTTGGATGTGTGTGATCTGTTCAGTTGCAAA |
| | | GGGTCACATGCTCAGAAGGACCGCATGCTAAATTTAATGCTTTGCAGTTACCCTCT |
| | | TGAAATCCTTTATTTTTTAAGAAGGAATTCGACATTTCCATTTTTCAATGAGCCCC |
| | | ACAAATTACGCAGCTAGTCCTGGGCTTCTCTACTCTGAAATTGGGCAGGATCTCTC |
| | | TTGATCTAGAATTTACTAAGGCATAATAGGGGCAAGAAAATCTTATGAAATAATGG |
| | | GGGGTAGGGAAGAGATGGGAATGGAGCATGAGATCCAGCTTCGTTATTCTCTACTT |
| | | GAGAAAAATAAGGCCCCAAAGATTAAACAACTTGCCCAAGGATATTGCTTGTTAGT |
| | | GTCAGAACTGAAACCAGAAACCAAATGATCATATCCCTAGACTTTTAGTCTGCTTT |
| | | CTCTTCCATAAAATGAAACTTATAATGTTTCTAATCCATTGCTCAGACAGGTAGAC |
| | | ATGAATATTAATTGATAATGACTATTAATTGATCTGGAAAATACTTGTTTGGGGAT |
| | | CAATAATATGTTTGGGCTATTATCTAATGCTGTGTAGAAATATTAAAACCCCTGTT |
| | | ATTTTGAAATAAAAAAGATACCCACTTTTTAT |
| 28 | GDF15 | AGTCCCAGCTCAGAGCCGCAACCTGCACAGCCATGCCCGGGCAAGAACTCAGGACG |
| | | GTGAATGGCTCTCAGATGCTCCTGGTGTTGCTGGTGCTCTCGTGGCTGCCGCATGG |
| | | GGGCGCCCTGTCTCTGGCCGAGGCGAGCCGCGCAAGTTTCCCGGGACCCTCAGAGT |
| | | TGCACTCCGAAGACTCCAGATTCCGAGAGTTGCGGAAACGCTACGAGGACCTGCTA |
| | | ACCAGGCTGCGGGCCAACCAGAGCTGGGAAGATTCGAACACCGACCTCGTCCCGGC |
| | | CCCTGCAGTCCGGATACTCACGCCAGAAGTGCGGCTGGGATCCGGCGGCCACCTGC |
| | | ACCTGCGTATCTCTCGGGCCGCCCTTCCCGAGGGGCTCCCCGAGGCCTCCCGCCTT |
| | | CACCGGGCTCTGTTCCGGCTGTCCCCGACGGCGTCAAGGTCGTGGGACGTGACACG |
| | | ACCGCTGCGGCGTCAGCTCAGCCTTGCAAGACCCCAGGCGCCCGCGCTGCACCTGC |
| | | GACTGTCGCCGCCGCCGTCGCAGTCGGACCAACTGCTGGCAGAATCTTCGTCCGCA |
| | | CGGCCCCAGCTGGAGTTGCACTTGCGGCCGCAAGCCGCCAGGGGGCGCCGCAGAGC |
| | | GCGTGCGCGCAACGGGGACCACTGTCCGCTCGGGCCCGGGCGTTGCTGCCGTCTGC |
| | | ACACGGTCCGCGCGTCGCTGGAAGACCTGGGCTGGGCCGATTGGGTGCTGTCGCCA |
| | | CGGGAGGTGCAAGTGACCATGTGCATCGGCGCGTGCCCGAGCCAGTTCCGGGCGGC |
| | | AAACATGCACGCGCAGATCAAGACGAGCCTGCACCGCCTGAAGCCCGACACGGTGC |
| | | CAGCGCCCTGCTGCGTGCCCGCCAGCTACAATCCCATGGTGCTCATTCAAAAGACC |
| | | GACACCGGGGTGTCGCTCCAGACCTATGATGACTTGTTAGCCAAAGACTGCCACTG |
| | | CATATGAGCAGTCCTGGTCCTTCCACTGTGCACCTGCGCGGAGGACGCGACCTCAG |
| | | TTGTCCTGCCCTGTGGAATGGGCTCAAGGTTCCTGAGACACCCGATTCCTGCCCAA |
| | | ACAGCTGTATTTATATAAGTCTGTTATTTATTATTAATTTATTGGGGTGACCTTCT |
| | | TGGGGACTCGGGGGCTGGTCTGATGGAACTGTGTATTTATTTAAAACTCTGGTGAT |
| | | AAAAATAAAGCTGTCTGAACTGTT |
| 29 | GLYATL1 | CAGTTGCCATGGGCCAGGAGTCTCACAGAACTACTAAGCAGCTGCTTACCCAATTT |
| | | TGGCTGGAGAGATAAGTACCCTCTGGGGCCACCACAGCTGGCCCCAACCATGGTCC |
| | | TGATTGCCTTGGAAGAACTGCCTTTGGAATTTCACATCGGCATCCAGATAGATGGT |
| | | GTCACAAGAAGGATCTGAAGTGGAGCTTCTAGTATCCCCAGGAGCGCGAAGTGAAC |
| | | ACGGAAGGTACCTGCAGGATCCAATTGTGTCCATTGATCTCTCAGAGTGGCTGAGG |
| | | ATAATAGAGTTTCTTCTTCAAGGTCTCAAGGTCTGAAGCATCCCACAGAATGATCC |
| | | TACTGAATAACTCCCATAAGCTGCTGGCCCTATACAAATCCTTGGCCAGGAGCATC |
| | | CCTGAGTCCCTGAAGGTGTATGGCTCTGTGTATCACATCAATCACGGGAACCCCTT |
| | | CAACATGGAGGTGCTGGTGGATTCCTGGCCTGAATATCAGATGGTTATTATCCGGC |
| | | CTCAAAAGCAGGAGATGACTGATGACATGGATTCATACACAAACGTATATCGTATG |
| | | TTCTCCAAAGAGCCTCAAAAATCAGAAGAAGTTTTGAAAAATTGTGAGATCGTAAA |
| | | CTGGAAACAGAGACTCCAAATCCAAGGTCTTCAAGAAAGTTTAGGTGAGGGGATAA |
| | | GAGTGGCTACATTTTCAAAGTCAGTGAAAGTAGAGCATTCGAGAGCACTCCTCTTG |
| | | GTTACGGAAGATATTCTGAAGCTCAATGCCTCCAGTAAAAGCAAGCTTGGAAGCTG |
| | | GGCTGAGACAGGCCACCCAGATGATGAATTTGAAAGTGAAACTCCCAACTTTAAGT |
| | | ATGCCCAGCTGGATGTCTCTTATTCTGGGCTGGTAAATGACAACTGGAAGCGAGGG |
| | | AAGAATGAGAGGAGCCTGCATTACATCAAGCGCTGCATAGAAGACCTGCCAGCAGC |
| | | CTGTATGCTCGGCCCAGAGGGAGTCCCGGTCTCATGGGTAACCATGGACCCTTCTT |
| | | GTGAAGTAGGAATGGCCTACAGCATGGAAAAATACCGAAGGACAGGCAACATGGCA |
| | | CGAGTGATGGTGCGATACATGAAATATCTGCGTCAGAAGAATATTCCATTTTACAT |
| | | CTCTGTGTTGGAAGAAAATGAAGACTCCCGCAGATTTGTGGGGCAGTTTGGTTTCT |
| | | TTGAGGCCTCCTGTGAGTGGCACCAATGGACTTGCTACCCACAGAATCTAGTTCCA |
| | | TTTTAGACAATGAAGCTGCTTAGTAATCTCTGCCAAGCCATCTCTTAATATTAAAG |
| | | CAGACACCACAGAATAGATTTCTTCACTTACAAATGCATATTGGGCACTTATAATA |
| | | CAGCAGGAACTCTTCTCACCTGGAGCCTTGATGTTAAAAGACACAGCCATGCTCTT |
| | | GAGGAGCTTACAATCCTGGCTGGAGGCAGGGGAGGGTATATTCTTTAAATATGCTT |
| | | AAGTGTTATAGGGAAAGACGGGGTTACCAGTAAACATGTAACTAGAAAGCCAGGCT |
| | | CAGTTCTTACCTCTGGGAATCAGAACTCTTTATGCAACTTGGTTAATAGAATCTAC |
| | | TATCTGGAAGATAAATGAAGGATTTTAATAAAATTTTCAATAGAATAAA |
| 30 | GOLM1 | ATCTTCACTTTTTCCGTTGCTAGCAGTGGAAGGGTCACAGACCAAACACTAAGGCC |
| | | TGAGCGGTGACAACCGAGGCGAGATGATGGTCAACAGGGAATGCCTCGTGGGAGAA |
| | | AAAAGACAATTTTATTCTCAGCGCTGATTTTGAGATGATGGGCTTGGGAAACGGGC |
| | | GTCGCAGCATGAAGTCGCCGCCCCTCGTGCTGGCCGCCCTGGTGGCCTGCATCATC |
| | | GTCTTGGGCTTCAACTACTGGATTGCGAGCTCCCGGAGCGTGGACCTCCAGACACG |
| | | GATCATGGAGCTGGAAGGCAGGGTCCGCAGGGCGGCTGCAGAGAGAGGCGCCGTGG |
| | | AGCTGAAGAAGAACGAGTTCCAGGGAGAGCTGGAGAAGCAGCGGGAGCAGCTTGAC |
| | | AAAATCCAGTCCAGCCACAACTTCCAGCTGGAGAGCGTCAACAAGCTGTACCAGGA |
| | | CGAAAAGGCGGTTTTGGTGAATAACATCACCACAGGTGAGAGGCTCATCCGAGTGC |
| | | TGCAAGACCAGTTAAAGACCCTGCAGAGGAATTACGGCAGGCTGCAGCAGGATGTC |
| | | CTCCAGTTTCAGAAGAACCAGACCAACCTGGAGAGGAAGTTCTCCTACGACCTGAG |
| | | CCAGTGCATCAATCAGATGAAGGAGGTGAAGGAACAGTGTGAGGAGCGAATAGAAG |
| | | AGGTCACCAAAAAGGGGAATGAAGCTGTAGCTTCCAGAGACCTGAGTGAAAACAAC |
| | | GACCAGAGACAGCAGCTCCAAGCCCTCAGTGAGCCTCAGCCCAGGCTGCAGGCAGC |
| | | AGGCCTGCCACACACAGAGGTGCCACAAGGGAAGGGAAACGTGCTTGGTAACAGCA |
| | | AGTCCCAGACACCAGCCCCCAGTTCCGAAGTGGTTTTGGATTCAAAGAGACAAGTT |
| | | GAGAAAGAGGAAACCAATGAGATCCAGGTGGTGAATGAGGAGCCTCAGAGGGACAG |
| | | GCTGCCGCAGGAGCCAGGCCGGGAGCAGGTGGTGGAAGACAGACCTGTAGGTGGAA |
| | | GAGGCTTCGGGGGAGCCGGAGAACTGGGCCAGACCCCACAGGTGCAGGCTGCCCTG |
| | | TCAGTGAGCCAGGAAAATCCAGAGATGGAGGGCCCTGAGCGAGACCAGCTTGTCAT |
| | | CCCCGACGGACAGGAGGAGGAGCAGGAAGCTGCCGGGGAAGGGAGAAACCAGCAGA |
| | | AACTGAGAGGAGAAGATGACTACAACATGGATGAAAATGAAGCAGAATCTGAGACA |
| | | GACAAGCAAGCAGCCCTGGCAGGGAATGACAGAAACATAGATGTTTTTAATGTTGA |
| | | AGATCAGAAAAGAGACACCATAAATTTACTTGATCAGCGTGAAAAGCGGAATCATA |
| | | CACTCTGAATTGAACTGGAATCACATATTTCACAACAGGGCCGAAGAGATGACTAT |
| | | AAAATGTTCATGAGGGACTGAATACTGAAAACTGTGAAATGTACTAAATAAAATGT |
| | | ACATCTGAAGATGATTATTGTGAAATTTTAGTATGCACTTTGTGTAGGAAAAAATG |
| | | GAATGGTCTTTTAAACAGCTTTTGGGGGGTACTTTGGAAGTGTCTAATAAGGTGTC |
| | | ACAATTTTTGGTAGTAGGTATTTCGTGAGAAGTTCAACACCAAAACTGGAACATAG |
| | | TTCTCCTTCAAGTGTTGGCGACAGCGGGGCTTCCTGATTCTGGAATATAACTTTGT |
| | | GTAAATTAACAGCCACCTATAGAAGAGTCCATCTGCTGTGAAGGAGAGACAGAGAA |
| | | CTCTGGGTTCCGTCGTCCTGTCCACGTGCTGTACCAAGTGCTGGTGCCAGCCTGTT |
| | | ACCTGTTCTCACTGAAAAGTCTGGCTAATGCTCTTGTGTAGTCACTTCTGATTCTG |
| | | ACAATCAATCAATCAATGGCCTAGAGCACTGACTGTTAACACAAACGTCACTAGCA |
| | | AAGTAGCAACAGCTTTAAGTCTAAATACAAAGCTGTTCTGTGTGAGAATTTTTTAA |
| | | AAGGCTACTTGTATAATAACCCTTGTCATTTTTAATGTACAAAACGCTATTAAGTG |
| | | GCTTAGAATTTGAACATTTGTGGTCTTTATTTACTTTGCTTCGTGTGTGGGCAAAG |
| | | CAACATCTTCCCTAAATATATATTACCAAGAAAAGCAAGAAGCAGATTAGGTTTTT |
| | | GACAAAACAAACAGGCCAAAAGGGGGCTGACCTGGAGCAGAGCATGGTGAGAGGCA |
| | | AGGCATGAGAGGGCAAGTTTGTTGTGGACAGATCTGTGCCTACTTTATTACTGGAG |
| | | TAAAAGAAAACAAAGTTCATTGATGTCGAAGGATATATACAGTGTTAGAAATTAGG |
| | | ACTGTTTAGAAAAACAGGAATACAATGGTTGTTTTTATCATAGTGTACACATTTAG |
| | | CTTGTGGTAAATGACTCACAAAACTGATTTTAAAATCAAGTTAATGTGAATTTTGA |
| | | AAATTACTACTTAATCCTAATTCACAATAACAATGGCATTAAGGTTTGACTTGAGT |
| | | TGGTTCTTAGTATTATTTATGGTAAATAGGCTCTTACCACTTGCAAATAACTGGCC |
| | | ACATCATTAATGACTGACTTCCCAGTAAGGCTCTCTAAGGGGTAAGTAGGAGGATC |
| | | CACAGGATTTGAGATGCTAAGGCCCCAGAGATCGTTTGATCCAACCCTCTTATTTT |
| | | CAGAGGGGAAAATGGGGCCTAGAAGTTACAGAGCATCTAGCTGGTGCGCTGGCACC |
| | | CCTGGCCTCACACAGACTCCCGAGTAGCTGGGACTACAGGCACACAGTCACTGAAG |
| | | CAGGCCCTGTTTGCAATTCACGTTGCCACCTCCAACTTAAACATTCTTCATATGTG |
| | | ATGTCCTTAGTCACTAAGGTTAAACTTTCCCACCCAGAAAAGGCAACTTAGATAAA |
| | | ATCTTAGAGTACTTTCATACTCTTCTAAGTCCTCTTCCAGCCTCACTTTGAGTCCT |
| | | CCTTGGGGTTGATAGGAATTTTCTCTTGCTTTCTCAATAAAGTCTCTATTCATCTC |
| | | ATGTTTAATTTGTACGCATAGAATTGCTGAGAAATAAAATGTTCTGTTCAACTTA |
| 31 | GRIN3A | AGTCGCGTTGCTCCTCCGAGGAAGCAAGCGGCGGTGGCGACTCGGTGGAAAAATAA |
| | | CGAAAGAAAGGCAGAGAGGAAGTAGCGAGAGAAGAGAGAAAATGAAGTCGGCGCTG |
| | | GGGGAGCCTGCAGGAGGGTGGCCAACAGTGGAGGAAGGTGGATTTGGCTTCTTTTC |
| | | CGCACCCCGGGCGTGAAAGCCCTCTCCAACGCGACCCCAGGAAATAAGTGGGTCTC |
| | | GCCTGGGCAGAAAAGGAAAAGAATCCAGGCGAGAGCGCGTCGCTCCTCTGTCACTG |
| | | CTGCCCCCGAGGAACTCCGGCTGCTTCTCATCCCGGCCGCCTCGCGGGGCCGGACG |
| | | CAGTGCCCGAGGCGCCCTGCAGATGGGGGGGGCAGGGAACGGGCGCTCCAGCTGCG |
| | | GGTGACAGGCGCCGGCCCGCCCGCCTGCCTGCTCAGCGCAGTGACCGGGCGGGCAG |
| | | AGGATGCCAGGCGGAGGGACCTGGGAGCGGGATCTGAGACTGCCGGAGGCGCGCTA |
| | | CGCTCCAACTTGCATGGCCTAGAGACCGCTCCAGCTCCTGGGACCGCTTCACCGAG |
| | | TGGAGTGAAGCTGCGCGCGGGACCTGGAGGCGGAGACCTCAGGCAGCGGCTGCAGA |
| | | GGGGCGAGCCGGGCGCAGGAGGGGGCGCGCTTTCTCCCTGCGGGTCTCAGTAATGA |
| | | GGAGACTGAGTTTGTGGTGGCTGCTGAGCAGGGTCTGTCTGCTGTTGCCGCCGCCC |
| | | TGCGCACTGGTGCTGGCCGGGGTGCCCAGCTCCTCCTCGCACCCGCAGCCCTGCCA |
| | | GATCCTCAAGCGCATCGGGCACGCGGTGAGGGTGGGCGCGGTGCACTTGCAGCCCT |
| | | GGACCACCGCCCCCCGCGCGGCCAGCCGCGCTCCGGACGACAGCCGAGCAGGAGCC |
| | | CAGAGGGATGAGCCGGAGCCAGGGACTAGGCGGTCCCCGGCGCCCTCGCCGGGCGC |
| | | ACGCTGGTTGGGGAGCACCCTGCATGGCCGGGGGCCGCCGGGCTCCCGTAAGCCCG |
| | | GGGAGGGCGCCAGGGCGGAGGCCCTGTGGCCACGGGACGCCCTCCTATTTGCCGTG |
| | | GACAACCTGAACCGCGTGGAAGGGCTGCTACCCTACAACCTGTCTTTGGAAGTAGT |
| | | GATGGCCATCGAGGCAGGCCTGGGCGATCTGCCACTTTTGCCCTTCTCCTCCCCTA |
| | | GTTCGCCATGGAGCAGTGACCCTTTCTCCTTCCTGCAAAGTGTGTGCCATACCGTG |
| | | GTGGTGCAAGGGGTGTCGGCGCTGCTCGCCTTCCCCCAGAGCCAGGGCGAAATGAT |
| | | GGAGCTCGACTTGGTCAGCTTAGTCCTGCACATTCCAGTGATCAGCATCGTGCGCC |
| | | ACGAGTTTCCACGGGAGAGTCAGAATCCCCTTCACCTACAACTGAGTTTAGAAAAT |
| | | TCATTAAGTTCTGATGCTGATGTCACTGTCTCAATCCTGACCATGAACAACTGGTA |
| | | CAATTTTAGCTTGTTGCTGTGCCAGGAAGACTGGAACATCACCGACTTCCTCCTCC |
| | | TTACCCAGAATAATTCCAAGTTCCACCTTGGTTCTATCATCAACATCACCGCTAAC |
| | | CTCCCCTCCACCCAGGACCTCTTGAGCTTCCTACAGATCCAGCTTGAGAGTATTAA |
| | | GAACAGCACACCCACAGTGGTGATGTTTGGCTGCGACATGGAAAGTATCCGGCGGA |
| | | TTTTCGAAATTACAACCCAGTTTGGGGTCATGCCCCCTGAACTTCGTTGGGTGCTG |
| | | GGAGATTCCCAGAATGTGGAGGAACTGAGGACAGAGGGTCTGCCCTTAGGGCTCAT |
| | | TGCTCATGGAAAAACAACACAGTCTGTCTTTGAGCACTACGTACAAGATGCTATGG |
| | | AGCTGGTCGCAAGAGCTGTAGCCACAGCCACCATGATCCAACCAGAACTTGCTCTC |
| | | ATTCCCAGCACGATGAACTGCATGGAGGTGGAAACTACAAATCTCACTTCAGGACA |
| | | ATATTTATCAAGGTTTCTAGCCAATACCACTTTCAGAGGCCTCAGTGGTTCCATCA |
| | | GAGTAAAAGGTTCCACCATCGTCAGCTCAGAAAACAACTTTTTCATCTGGAATCTT |
| | | CAACATGACCCCATGGGAAAGCCAATGTGGACCCGCTTGGGCAGCTGGCAGGGGGG |
| | | AAAGATTGTCATGGACTATGGAATATGGCCAGAGCAGGCCCAGAGACACAAAACCC |
| | | ACTTCCAACATCCAAGTAAGCTACACTTGAGAGTGGTTACCCTGATTGAGCATCCT |
| | | TTTGTCTTCACAAGGGAGGTAGATGATGAAGGCTTGTGCCCTGCTGGCCAACTCTG |
| | | TCTAGACCCCATGACTAATGACTCTTCCACATTGGACAGCCTTTTTAGCAGCCTCC |
| | | ATAGCAGTAATGATACAGTGCCCATTAAATTCAAGAAGTGCTGCTATGGATATTGC |
| | | ATTGATCTGCTGGAAAAGATAGCAGAAGACATGAACTTTGACTTCGACCTCTATAT |
| | | TGTAGGGGATGGAAAGTATGGAGCATGGAAAAATGGGCACTGGACTGGGCTAGTGG |
| | | GTGATCTCCTGAGAGGGACTGCCCACATGGCAGTCACTTCCTTTAGCATCAATACT |
| | | GCACGGAGCCAGGTGATAGATTTCACCAGCCCTTTCTTCTCCACCAGCTTGGGCAT |
| | | CTTAGTGAGGACCCGAGATACAGCAGCTCCCATTGGAGCCTTCATGTGGCCACTCC |
| | | ACTGGACAATGTGGCTGGGGATTTTTGTGGCTCTGCACATCACTGCCGTCTTCCTC |
| | | ACTCTGTATGAATGGAAGAGTCCATTTGGTTTGACTCCCAAGGGGCGAAATAGAAG |
| | | TAAAGTCTTCTCCTTTTCTTCAGCCTTGAACATCTGTTATGCCCTCTTGTTTGGCA |
| | | GAACAGTGGCCATCAAACCTCCAAAATGTTGGACTGGAAGGTTTCTAATGAACCTT |
| | | TGGGCCATTTTCTGTATGTTTTGCCTTTCCACATACACGGCAAACTTGGCTGCTGT |
| | | CATGGTAGGTGAGAAGATCTATGAAGAGCTTTCTGGAATACATGACCCCAAGTTAC |
| | | ATCATCCTTCCCAAGGATTCCGCTTTGGAACTGTCCGAGAAAGCAGTGCTGAAGAT |
| | | TATGTGAGACAAAGTTTCCCAGAGATGCATGAATATATGAGAAGGTACAATGTTCC |
| | | AGCCACCCCTGATGGAGTGGAGTATCTGAAGAATGATCCAGAGAAACTAGACGCCT |
| | | TCATCATGGACAAAGCCCTTCTGGATTATGAAGTGTCAATAGATGCTGACTGCAAA |
| | | CTTCTCACTGTGGGGAAGCCATTTGCCATAGAAGGATACGGCATTGGCCTCCCACC |
| | | CAACTCTCCATTGACCGCCAACATATCCGAGCTAATCAGTCAATACAAGTCACATG |
| | | GGTTTATGGATATGCTCCATGACAAGTGGTACAGGGTGGTTCCCTGTGGCAAGAGA |
| | | AGTTTTGCTGTCACGGAGACTTTGCAAATGGGCATCAAACACTTCTCTGGGCTCTT |
| | | TGTGCTGCTGTGCATTGGATTTGGTCTGTCCATTTTGACCACCATTGGTGAGCACA |
| | | TAGTATACAGGCTGCTGCTACCACGAATCAAAAACAAATCCAAGCTGCAATACTGG |
| | | CTCCACACCAGCCAGAGATTACACAGAGCAATAAATACATCATTTATAGAGGAAAA |
| | | GCAGCAGCATTTCAAGACCAAACGTGTGGAAAAGAGGTCTAATGTGGGACCCCGTC |
| | | AGCTTACCGTATGGAATACTTCCAATCTGAGTCATGACAACCGACGGAAATACATC |
| | | TTTAGTGATGAGGAAGGACAAAACCAGCTGGGCATCCGGATCCACCAGGACATCCC |
| | | CCTCCCTCCAAGGAGAAGAGAGCTCCCTGCCTTGCGGACCACCAATGGGAAAGCAG |
| | | ACTCCCTAAATGTATCTCGGAACTCAGTGATGCAGGAACTCTCAGAGCTCGAGAAG |
| | | CAGATTCAGGTGATCCGTCAGGAGCTGCAGCTGGCTGTGAGCAGGAAAACGGAGCT |
| | | GGAGGAGTATCAAAGGACAAGTCGGACTTGTGAGTCCTAGGTGACCACACTGCTTC |
| | | CCTTTCTCAGTTCCTGACCTTCCTCTGAGCCCTTGAGACACTTTGTAATGCTCTTT |
| | | TGTAACTATCGACAAAGGTGTGGGGAAGCTGAGGTCTAGGTCTTCTTAAAGGTCAA |
| | | GTCTGCTCTCCCTCGCCTAAAGTGCAGCAGCAGCTCCTCTCAAGCTCACTCTCTAG |
| | | GTCTCCAGGGTAGGAGTGTTTTTCTAGCAAGAATCTTAGTCAGGAGTAAGCTCTGT |
| | | GCGAGAGATCTGTGAATAACCAGATAACCCCAGCTGCCGTTAACCTTTTCACCAGG |
| | | TGCCACAGTAATATTTCTGGTTTTTAGCCCTTTCTCTGCACTACCAACAAGAGATA |
| | | AAATTGTTACTCACACTTATGTCTTACTGGGTTGCTGGTTTTCATCGTAACACAGA |
| | | ACGAGGTTATCTAGGGTTGTAGCTTTTGATACAACTCCCCGATCTAGATTTATTCC |
| | | TACATTCTGAATGGGGAGCAGGTAAGAGCAGAGCACCTCCCACTGGGGGTGGGGTA |
| | | TTTAAAAATTAACTCATTAGTATCATAAACGTCAAGGATTGATTGGACCAGGCAAG |
| | | AGCCATGTTTTTGAGAAGGTTCTGGATCTCTGACTCCATCCTGACTGTTTAGTAAG |
| | | AGCATGCTTACACCCTACTGTGAAAAGGGGAGGGGATGTGGTAAGCGGAAACAGAA |
| | | GACAGGCAGCAGAGGCATTAAAAATGCATACCATGCTTTCAGAACAAAAGCTCTGG |
| | | GCCAGAAAGGCAATTTGGCTAAAAAATGAATAAGACTACTTCTAATGTAACTAAGC |
| | | ATCTCCACTATGGTGTGTGCCTTTTATAAAGGAAAAGAGAGAAAAAGGCAAAGCAA |
| | | GGTTGTGGCCTTAGGTTGGACCTGGAATATCCCTTATTGCCTATAATGGAATATGT |
| | | GACACTGTGGGTGAAATGTTCTACACACCACACACTAGGCCATTTTCAGATCAGCA |
| | | GTCACCCATCGCTTAGCATAGAAATCCCAAAACCTCCAGCCCGGGAACACTATAAG |
| | | CTTCGACCATTCAGGAATCTGCCCTGCACTTTGCATATCTGTATAGAAAATCAAGT |
| | | CAATCCCCCATCCTCACACCCACTCATCTCTGAGGAGCTATGAACTGGTTTTGGTC |
| | | CCTCTAATGATCCTCCAGCCTCATCTAATGCCCCCCAAAGACTGATACAAGTAACC |
| | | TCCCCTCTGCTTAGGTGTCACTTTCTCAGCATATCAAGTTTAGGCAGCAAGGGAAA |
| | | GGAATATGGGTCAGTTCTCAAATGTCAATGTAGATAAGAGTCATCTAGTAGAGAAC |
| | | TCATCAGAGTGCGGATTGCCAAGACCCTTCTCCAGAGATTATGGGGTTGGGGGTGG |
| | | AGGTCTAGAGGTGAGCTCAGAAACCTACTGTTAACCAACACCCCCAAGTGACTGAC |
| | | ACAGGTGGTCTAAAAATTACTTTTCTAGAAACACCATTCTGGAAGTTTGGCTGCCC |
| | | ACAGGCAGGAGGAGAAGCATGAAGAGAAAACCTGTTTGAGAAGTTTTGTTTTGTTT |
| | | TGTTTTGCTTTTTAATAATTTTAGCACACATCTGCTGACTCTCCTTCAACATCCTC |
| | | ACCCCCACCCCTGGGCACCATTTAGGACAAGACTTCCTTATTTATCAATTACTTGA |
| | | TTTATCTTCTCAGGACTCATTGTTCCACCCCCAACCAATTTGAATGCCTACAATAA |
| | | GTTCAGGAGCTGTGCCAAGCACTTTCCTCTTTTACAGCTGGAGATCACTGGAAAGG |
| | | TGTCTCAGTCACAAAACTTCTCCCTCTACTACTGGATGAAATGTCTGCATTTCCAC |
| | | CAAAATCTACCCAGTCACCCAGGGAATAACAACTTAAGCTGTAGTTAGATAACACC |
| | | TAGTGATTAATTGGCTGAGAAAACCCTGGAGTGGAGGGAGGCTCAGAGATACTGAT |
| | | ATGGATGTGGGAGGGCTCTAAAGTTAGAGGTCACCAACTCCACAGATGAAACAGTT |
| | | CAATAATGAGGAAACAGGTGAGCCCTGAAAACACAAAAGGACAGTTCTGTGTTGAA |
| | | ACACCCCATCCCCTCACGTTCTCACCCCAGGCCCAGAAGTAGGTTGCAACTGCCTT |
| | | TGGAAGATTTTGCCCCTTAGCCATCCCCACCCACTTGTACCAGCTAAGAATGCTGG |
| | | AGACTCTGCCACCATGCTCTGCGTGCCCCTGAACCTCTGTGCAGCCCGGAAGGCTG |
| | | ATGTACAGGTGTACCTCAATCCACATTACAGCCATGCTCCTAATGTACATGGACAT |
| | | TTTTGTAACTCAGCTCATATTCTGACTGTATTTGAGAAGCTGGCTGTTTAAGGGAA |
| | | CCCAGAAGTGAATTCTTTTGTAAAGTAAAGCACCCTTTTGTAATGCAATTAATTAT |
| | | CCCTTAATGTATCTGTTTTGTAAGTCTGCATTTTTGTATATCGGATTTACCTTAAG |
| | | CTTCTCTAGTGAGGCATTCTGAGCAGTGGTGATCACATGCCAGATCGCCCTGCCTA |
| | | TCCACAAAGTAGATGACCAATGCACGCTCCTCAAACATCTTTGGAGGAACTACCTG |
| | | GCCAAAACACTGGCCAGGATGCAGCAAGCAGCAGCAGGGGCTGACAGCAGGCTTAC |
| | | TGCCATCAACATTGCTTGAAATGCCTCTATGTTCTGAATAAAGAAAAACCATAATT |
| | | GCTTGTGGTGAAACGAAGCAGTCTTCATGTTAAGTAGCAATGGTTATTTTTATTGG |
| | | TAGTAACTGAACAGTGTTTTGCAATTTGTGAAACAGTGTATTGTGTTTTGTAAAAT |
| | | GATGTCATGAAATGGTGGGTCCTTGGAAACCTCCTTTCCGTTCAGCTCTGCCTCTG |
| | | TTCTTTCAACTCCTTTGAGGCTCAAAAAAAACACAAAGATCAGAAGCCTTCAGATA |
| | | GAGGGTGGTATTCTGGTAAAGAAGAAAGAGATAAGGGACGCTACCTTGCTTTTCTG |
| | | GCACAGGAAGCACATGATAAAGCATGCTCAGATGAGCTGGAACAGATATAGCTACC |
| | | TGGTTCGTGTAAATAAGAATAATCAAGGCCCCAGAGTGTGTATGCTTCCAGGTGGA |
| | | GGAGAAAGGGGAATCTCCCAAAATTTAAAAACAAATTGGAAGAATAACCAGGACAG |
| | | CCAAGTGAAGCAGCCACAGGGACCCAAGCAGTCGAGGTCTTTAATGTGCCTGGAGA |
| | | TGACTCTCTGCTATTCATGAATCTTGCTATTGCACAAACCCTATCAAGAGCTGCTG |
| | | CTTCCCTTCCAGCCAGAAAAGTGGTAAGCGGAGCAAGTGCCAAGCAGAACAGACCT |
| | | TATCATCTGGGTAACAGACTTCTCAGTGTTGGTGCTGTGTCTGTTAGAGCCTTAGA |
| | | GCAAGTTAAGCACTTCCTTGGTGTGGGTAAAGAATAAAGGGGAAAGAAACTACTTT |
| | | AGAGCCTCTTTTTCTCCCAACTCATATTTTTGATAGGAAAAACAGAAAACCCATCC |
| | | AGTTCTTCAGAAATTGCTTTCTAGGCATTAATACTACTTTACTATCTATACTGITT |
| | | AGTTATTCCTTTCTTTACCCACCTAAACTATCCATCTAATCCAGGATTCCCTCACT |
| | | CTTTTTTTTTAGTTACTAATCATTTTATGAAAATAATGTATTTATAAGTATTTTCT |
| | | TAAGGTTTGTGAAGAGTATTTGCATTGTGTCTTCATTTTAATGTGTTTGCAATCGC |
| | | TCCGCTCCAGGAAGAACGGAAATGCTGTCTTGTGAGCATGAAGTGAACGGGCTGTT |
| | | TTGCTCCAGCCACTTTTCTTGTACAACCACATGGATGGATTAGATGTCCTCAGGTC |
| | | TTTTCCATCTTCAGTTTCTATGACTGTGGAATAAATGTTCAGATAGAAACTTCA |
| 32 | LINC00993 | TTGAAGAGATGAGTGCGGGGCTCATCTATCCCTGGAATTGTCTTTCCCACAATCCC |
| | | TGACACAGAATATGAGCCATACAGGAATTCTGAAGAAATGGGTCTCTTGCCACCTC |
| | | CCAGTAAAAGATTATTTTTTAAAAAAAAAAGGCTCTGCTTTGACCTGAAGTATTTT |
| | | ATCTATCCTCAGTCTCAGGACACTGTTGATGGAATTAAGGCCAAGCACATCTGCAA |
| | | AAAAGACATTGCTGGAGGAGGTGCAAAGAGCTGGAAACCAAGTCTCCAGTCCTGGG |
| | | AAAAGCAGTGGTATGGAAAAGCAATGGAAAGAGCATTTTGAAAATGCCATTCCACT |
| | | GTTTTCTGGCCTTTATGATTTCTGCTGAGAAATCCACTGTTAGTCTGATGGGGTCT |
| | | CCTTCATAGCACCAATGACCTGAAGAGCCTTGTTGAAGGAAGACTCCATCTGATGA |
| | | CTCAGAGCAAGTATTTTTTAGTGTGTTATTGTTATTAGCAGAAAGAGGGCCATAAA |
| | | ATACATGGGGCAAGCTGAATATATCTTAGGCAAAAGAAGAAAATATTCAAATTCTT |
| | | ATGTTATTTTATCTAATTATTTTATCTCTTTTTGTGTGTGACTTATAATGTGTGTA |
| | | TTGTATTAATAAAAGTATATAAACATGTAGTTTACAAAAAAAAAAAAAAA |
| 33 | LRRN1 | GAGCACAAAGCGGGGCGCACCGCGGGCGCCGGCAACGAGCCGGTGAACGAGGCGAG |
| | | GCCCGTGCGCCCGCGGCTGCAAGCGCCCGCCTGGCGGGGAGAGGGGCCGACGGCGT |
| | | CAGCCCGGGCGGCGGCATCCCTAGGCGCCTGGGGCGCCTTCCTCCGGACCTGGCCG |
| | | CTCGCTGCCCCGCCCTCTGCACCCCACTTCTCCGACCCTCCTTCCCAGTCCTGCCT |
| | | CCCCCTGCCCTGGCCTCTGAGAGCCGACTGAGCCCAGCCCCGTGCAGCAGCGGTTG |
| | | CCTGTGTCGCCGCCTAGTCTCCGGTCTTGGTGCTCTCCCGGGGGTGCCCCAAGGAG |
| | | CCAGTGCGCGCTGCGGGCTGGGAAGGAGGCGCCGCTCAGCTAGTCCTCCTCCTCCT |
| | | CCTCGTCTTTCTCCTCCTCCTGCTGCTGCTGCCGCCGCCGCCGCCGTGGGTGCCGG |
| | | GTCCGCGCGCACCCCAACACCCCCACCAGCTGGGCCTCGGGGGAGTCCCTGCCTGG |
| | | CAAGCTGGGGCTCGGGGAAAAGTCCAGCAGCGAGAGGGCCGCGTGTCCCGGGACGG |
| | | TTCCAGGGGAGCCCGCGCGGAAACGCGGACCAGGCGCTTCGGCCCGGATGCCGGAT |
| | | CCAGGAGAAGGGGACCTCGCTTGCCGTGTCCACCGCCGCGGCCCCAGCGCCAAGAA |
| | | CAGAGCCTGGCCGTGAGTGAACGACTGGCCGGGGTACAAATGGTTCTGAAGCACTT |
| | | GAAGCTCACAGGCAAAGGCTAAGAGTCTAAATCCCATCCCGCCGCACATCTCCAGA |
| | | CTTCAATTTGGCTGAAATAATTCATGCCACGGACCTGTGCACATGCCTGGAATTGA |
| | | GAGACACAGTTAAAAGACTCCAAGTTGCTTTCTGCCTTTTGAAAACTCCTGAAAAC |
| | | CATCCCTTTGGACTCTGGAATTCTACACAGCTCAACCAAGACTTTGCTTGAATGTT |
| | | TACATTTTCTGCTCGCTGTCCTACATATCACAATATAGTGTTCACGTTTTGTTAAA |
| | | ACTTTGGGGTGTCAGGAGTTGAGCTTGCTCAGCAAGCCAGCATGGCTAGGATGAGC |
| | | TTTGTTATAGCAGCTTGCCAATTGGTGCTGGGCCTACTAATGACTTCATTAACCGA |
| | | GTCTTCCATACAGAATAGTGAGTGTCCACAACTTTGCGTATGTGAAATTCGTCCCT |
| | | GGTTTACCCCACAGTCAACTTACAGAGAAGCCACCACTGTTGATTGCAATGACCTC |
| | | CGCTTAACAAGGATTCCCAGTAACCTCTCTAGTGACACACAAGTGCTTCTCTTACA |
| | | GAGCAATAACATCGCAAAGACTGTGGATGAGCTGCAGCAGCTTTTCAACTTGACTG |
| | | AACTAGATTTCTCCCAAAACAACTTTACTAACATTAAGGAGGTCGGGCTGGCAAAC |
| | | CTAACCCAGCTCACAACGCTGCATTTGGAGGAAAATCAGATTACCGAGATGACTGA |
| | | TTACTGTCTACAAGACCTCAGCAACCTTCAAGAACTCTACATCAACCACAACCAAA |
| | | TTAGCACTATTTCTGCTCATGCTTTTGCAGGCTTAAAAAATCTATTAAGGCTCCAC |
| | | CTGAACTCCAACAAATTGAAAGTTATTGATAGTCGCTGGTTTGATTCTACACCCAA |
| | | CCTGGAAATTCTCATGATCGGAGAAAACCCTGTGATTGGAATTCTGGATATGAACT |
| | | TCAAACCCCTCGCAAATTTGAGAAGCTTAGTTTTGGCAGGAATGTATCTCACTGAT |
| | | ATTCCTGGAAATGCTTTGGTGGGTCTGGATAGCCTTGAGAGCCTGTCTTTTTATGA |
| | | TAACAAACTGGTTAAAGTCCCTCAACTTGCCCTGCAAAAAGTTCCAAATTTGAAAT |
| | | TCTTAGACCTCAACAAAAACCCCATTCACAAAATCCAAGAAGGGGACTTCAAAAAT |
| | | ATGCTTCGGTTAAAAGAACTGGGAATCAACAATATGGGCGAGCTCGTTTCTGTCGA |
| | | CCGCTATGCCCTGGATAACTTGCCTGAACTCACAAAGCTGGAAGCCACCAATAACC |
| | | CTAAACTCTCTTACATCCACCGCTTGGCTTTCCGAAGTGTCCCTGCTCTGGAAAGC |
| | | TTGATGCTGAACAACAATGCCTTGAATGCCATTTACCAAAAGACAGTCGAATCCCT |
| | | CCCCAATCTGCGTGAGATCAGTATCCATAGCAATCCCCTCAGGTGTGACTGTGTGA |
| | | TCCACTGGATTAACTCCAACAAAACCAACATCCGCTTCATGGAGCCCCTGTCCATG |
| | | TTCTGTGCCATGCCGCCCGAATATAAAGGGCACCAGGTGAAGGAAGTTTTAATCCA |
| | | GGATTCGAGTGAACAGTGCCTCCCAATGATATCTCACGACAGCTTCCCAAATCGTT |
| | | TAAACGTGGATATCGGCACGACGGTTTTCCTAGACTGTCGAGCCATGGCTGAGCCA |
| | | GAACCTGAAATTTACTGGGTCACTCCCATTGGAAATAAGATAACTGTGGAAACCCT |
| | | TTCAGATAAATACAAGCTAAGTAGCGAAGGTACCTTGGAAATATCTAACATACAAA |
| | | TTGAAGACTCAGGAAGATACACATGTGTTGCCCAGAATGTCCAAGGGGCAGACACT |
| | | CGGGTGGCAACAATTAAGGTTAATGGGACCCTTCTGGATGGTACCCAGGTGCTAAA |
| | | AATATACGTCAAGCAGACAGAATCCCATTCCATCTTAGTGTCCTGGAAAGTTAATT |
| | | CCAATGTCATGACGTCAAACTTAAAATGGTCGTCTGCCACCATGAAGATTGATAAC |
| | | CCTCACATAACATATACTGCCAGGGTCCCAGTCGATGTCCATGAATACAACCTAAC |
| | | GCATCTGCAGCCTTCCACAGATTATGAAGTGTGTCTCACAGTGTCCAATATTCATC |
| | | AGCAGACTCAAAAGTCATGCGTAAATGTCACAACCAAAAATGCCGCCTTCGCAGTG |
| | | GACATCTCTGATCAAGAAACCAGTACAGCCCTTGCTGCAGTAATGGGGTCTATGTT |
| | | TGCCGTCATTAGCCTTGCGTCCATTGCTGTGTACTTTGCCAAAAGATTTAAGAGAA |
| | | AAAACTACCACCACTCATTAAAAAAGTATATGCAAAAAACCTCTTCAATCCCACTA |
| | | AATGAGCTGTACCCACCACTCATTAACCTCTGGGAAGGTGACAGCGAGAAAGACAA |
| | | AGATGGTTCTGCAGACACCAAGCCAACCCAGGTCGACACATCCAGAAGCTATTACA |
| | | TGTGGTAACTCAGAGGATATTTTGCTTCTGGTAGTAAGGAGCACAAAGACGTTTTT |
| | | GCTTTATTCTGCAAAAGTGAACAAGTTGAAGACTTTTGTATTTTTGACTTTGCTAG |
| | | TTTGTGGCAGAGTGGAGAGGACGGGTGGATATTTCAAATTTTTTTAGTATAGCGTA |
| | | TCGCAAGGGTTTGACACGGCTGCCAGCGACTCTAGGCTTCCAGTCTGTGTTTGGTT |
| | | TTTATTCTTATCATTATTATGATTGTTATTATATTATTATTTTATTTTAGTTGTTG |
| | | TGCTAAACTCAATAATGCTGTTCTAACTACAGTGCTCAATAAAATGATTAATGACA |
| | | GGATGGGGTTCCCCTGTGCTTTTACCAGTAGCATGACCCCTTCTGAAGCCATCCGT |
| | | AGAAAGTACTTTGTCCTCCAAAAAGCTAACATACGGTTTTGAAGCAGCATTGAAAC |
| | | TTTTGTAGCAATCTGGTCTATAGACTTTTAACTCAAGAAGCTAAGGCTAGACTTGT |
| | | TACCTTCGTTGAATGATGTTAGTTGACTGTACTGTAATGTTGTATCAACTGAATTG |
| | | AATGTTTGCCTTTAAACAATGAATTTTCTTTTTCTTTCCTTTTTTTTTTTTTTGTT |
| | | GTAATAGTTAAAGAGGCTTAGAACAAGCTAACAGGCAATAGAAATATGTATATCAG |
| | | ATTTTTTAATGTAACAAACTACATGTTAATTGTTATCTTATTCTTTTTATCTTTAG |
| | | TAGACACTTTTAAAAGAAAAGACAAGTTTGTTGTGTTTAACTCACCAACACGTGGT |
| | | GTATAATGAAGACAGAACTATAATAAATTAGTTTTGTTCTGATTTTTTAGAACACT |
| | | TGCAATAATGTATCATTTATAGTTCTTGCTAGTTGCAGTGGTAATATTTTTCACAT |
| | | CCATAAAAACAACTACCAAAATAAATCAGCTGTAGCATGTTGCTTTTTAAAGCTAG |
| | | GCCCTAAAAGGTTTTAATTCTTTTTCTAAGGGAAGAAATGTCTATTTTAATTAAGA |
| | | TATTTTAATGAACAGGATTTCTGTATTTTAAATAGTACTGACTAGCACCTAATGGG |
| | | CAGTGGGAGGGTGGTTCATATGAAGAAAAAAAGGTGTATTGTTGTATCCCATGCAT |
| | | AAATAAAGGTAAATATATATATACACCAATATATTCATATATACTCACACACATCC |
| | | CAACCTGTCACACACAATGCGTGTGTATATATATATGAATATATTGGATATGTCAT |
| | | TTCTGTAAGAGTTTTGTTAAAACCTGATTTTCTTTTGTAGTATCCACATTCTTCAT |
| | | CAAAGTACAAAAACGTCTGTGGAGTGTCACAAACTGTATGACATGTTATTTCTTTT |
| | | TAACAGTTGTCTATATGCTTAGACCCGTGTTAGTCTCTATATCTGTGTGGCAATAT |
| | | CTGCTGAGACAAGTAAATGATTAATAGAAAACAAAACAACTTCCGTACAGTTCAAA |
| | | CTTTTCATCCAAATATATATAGACAGTTTTGGAGAATTGTTTCAAGATTATAGAGG |
| | | AAACATGTAACATTTAGAGCAGATGGAACTAGGTTTAGGTAGAAGGCCAGTTCCAC |
| | | AAAGGGCAGAGGGAGGGATGGGATTTAATAGGTAAAGAAGAACCCATTTGAAAATA |
| | | AAGGTTGTTTCAAAAGGCAGCTGCCGCCAGGCACACAGCATTCCATCAGACAGGTG |
| | | CCAGACAAGCAAAAGCAAGGAAGTTGGCAGAAAGAAAGTCCAGGTGATGTAGGTTG |
| | | AGGTATTTCTTTCTTTGGCAGTATCTTGCTTTTGTGAATCACTTTATTAAAAAAAA |
| | | TCACTTTCTTCCCATTCTTAAGGGGTTTTGGCAAACAGAATTTCAGATCTTGAAAC |
| | | AAATGAGCTGCAACAGAAAAATAAGTACCTGAGCTCGAGGTATCCCTTCCCAAAGC |
| | | CTTCACTTATTGGTGAGAAAAACCTGGGCCCAGGGAGGGCTTGCAGCTTACCCAGT |
| | | TCGGACTCCTGCCAGTTCAGCGCTCTGCACTCCATTGATTGTTCTAGGCCCGGCAG |
| | | CCTGTTAGGTTCCTGTGCAGGGCCCCTCTTTTGCAGTTCTGAATAACCTCTAGTGC |
| | | CCCTGCTTCAAAAATGCAGTACATCCTCAAGTTCCCATTTACTCAGGATACATTTT |
| | | AGCACAGGGCAGATCAGACGGTGTCTCTACTGTTAGTATTGCAAAGTATGTATGGG |
| | | AAACACAGAGAATTGGAGCTGCGTTGAATGCAAACTTGAGGTGTTTCCCTTGAGGA |
| | | ATTCTTGTCTTCAAACGTCTGCAGAGTAATGGACCATGTTACAACTTTCCTGTTCA |
| | | TCTGTGAACCATGAAAATGGATGGCACTGATGCATTAGACCCTCAGCAGCCTGCAA |
| | | TTGCAAATCTGCGAGGTTTCATTCGGCCCATAAAGCAAACATTTGAACTTACACAG |
| | | AATGAGCACTTAAATACGGGTGCAATAAATGAAGGGAAAAACCTCAGCCGTTTCTC |
| | | CATTCTGAAGATATAGCAAGCACCGGGAAATCTAAGATTTTTCATCAACAATATCT |
| | | TCTGCCAGCCCAGTTTGGGGGGAAAAACCCCTTTTACATTTTTCTTCAGTAAAGTA |
| | | CTGGAACTTACTTTTCCCTGTCTGTGCTAATGAGCTGATTTTCAGCTGATAGAAAA |
| | | CAAAATGATAGAGTACTTTTTTCCTTGGCCAAGTATTTTCTCATTTGTATTTAATT |
| | | TCATAAATTAGACAGCCAGTGAAATTAGACCTCAAACTAGGTCCTGATGGATAATG |
| | | AATGTTATGTCACCTTTAACAGTGAAGTGGTTATTATAGGTCACTTTCTAATTTCA |
| | | TATTTTCCCTTTTGCTTTCTGCTGCCTTCAGGGTATATAGTGTATCTCTAACCTGA |
| | | TTTTTCAAGGTTATTTTTGGAGCAGTTTCTTAAAACAGGCATTCCCTAACTTGCTC |
| | | ATTTAATTAATGAAAAATTGAACTGATGCCATGGATATAAAAACAAATGTAATGTT |
| | | TGATTGTCAGTGTTTCTGATTTGGCAAAAAGGAATCATCTCTATTTTTTTGCAAAC |
| | | AATATCAAAGTGCATATTTTCTCTCA |
| 34 | MIPEP | GGAAACGCGGAGCGCGCGCTCCCAGCGAAAGCAGCAGGGCAGGGATCTGCGTTGGA |
| | | GGAAGGGACTGCTCTGGTGCTAGAATGCTGTGCGTCGGAAGGCTGGGCGGCTTGGG |
| | | AGCCAGAGCAGCAGCTCTGCCGCCCCGCCGGGCGGGCCGGGGAAGCCTCGAAGCCG |
| | | GGATCCGGGCCCGAAGGGTCAGCACCAGCTGGTCTCCCGTGGGCGCCGCCTTCAAT |
| | | GTCAAGCCCCAGGGCAGCCGCTTGGACCTGTTCGGCGAGCGCCGGGGTCTTTTTGG |
| | | AGTTCCTGAGCTGAGTGCCCCAGAAGGATTTCATATTGCACAAGAAAAAGCCTTGA |
| | | GAAAGACAGAATTGCTTGTGGACCGTGCATGTTCCACCCCACCTGGGCCCCAGACC |
| | | GTGCTGATCTTCGATGAGCTCTCGGATTCCTTATGCAGAGTGGCCGACTTGGCTGA |
| | | TTTTGTGAAAATCGCTCACCCTGAGCCAGCATTCAGAGAAGCTGCGGAAGAAGCTT |
| | | GTAGAAGTATTGGCACCATGGTAGAGAAGTTGAACACAAATGTGGATTTATATCAA |
| | | AGTTTGCAAAAATTACTAGCTGATAAAAAACTTGTGGATTCCCTTGATCCAGAAAC |
| | | AAGGCGAGTGGCTGAACTGTTTATGTTTGATTTTGAAATTAGTGGAATCCATCTAG |
| | | ACAAAGAAAAGCGTAAAAGAGCAGTGGACCTCAATGTTAAAATCTTGGATTTGAGT |
| | | AGTACATTTCTTATGGGAACCAATTTTCCCAACAAGATTGAGAAGCATCTCTTACC |
| | | AGAACACATTCGTCGTAACTTTACATCTGCTGGGGATCATATCATAATTGATGGTC |
| | | TCCACGCAGAATCACCAGATGACTTGGTGCGAGAAGCTGCTTATAAAATTTTTCTT |
| | | TATCCCAATGCTGGTCAATTGAAATGTTTAGAAGAATTGCTCAGCAGCAGAGATCT |
| | | TCTGGCAAAGTTGGTGGGGTATTCCACGTTTTCTCACAGGGCTCTCCAAGGAACGA |
| | | TAGCTAAAAATCCAGAGACTGTCATGCAGTTCCTTGAAAAACTATCTGACAAACTT |
| | | TCTGAAAGAACTCTGAAAGATTTTGAGATGATACGAGGGATGAAAATGAAACTGAA |
| | | TCCTCAAAATTCCGAAGTAATGCCCTGGGACCCCCCTTACTACAGTGGTGTGATTC |
| | | GTGCAGAAAGGTATAATATTGAGCCCAGCCTATATTGCCCGTTTTTCTCTCTTGGA |
| | | GCATGCATGGAAGGCCTGAATATTTTGCTTAACAGACTGTTGGGGATTTCATTATA |
| | | TGCAGAGCAGCCTGCAAAAGGAGAGGTGTGGAGCGAAGATGTCCGAAAACTGGCTG |
| | | TTGTTCATGAATCTGAAGGATTGTTGGGGTACATTTACTGTGATTTTTTTCAGCGA |
| | | GCAGACAAACCACATCAGGATTGCCATTTCACTATCCGTGGAGGCAGACTAAAGGA |
| | | AGATGGAGACTATCAACTCCCAGTTGTAGTTCTTATGCTGAATCTTCCCCGTTCCT |
| | | CAAGGAGTTCTCCAACTTTGCTAACTCCTAGCATGATGGAAAATCTTTTCCATGAA |
| | | ATGGGACATGCCATGCATTCAATGCTAGGACGTACTCGTTACCAACACGTCACTGG |
| | | GACCAGGTGCCCTACTGATTTTGCTGAGGTTCCTTCTATTCTGATGGAGTACTTTG |
| | | CAAATGATTATCGAGTAGTTAACCAATTTGCCAGACATTATCAGACTGGACAGCCA |
| | | CTGCCAAAAAATATGGTGTCTCGTCTTTGTGAATCTAAAAAGGTTTGTGCTGCAGC |
| | | TGATATGCAACTTCAGGTCTTTTATGCCACTCTGGATCAAATCTACCATGGGAAGC |
| | | ATCCCCTGAGGAATTCAACCACAGACATTCTCAAGGAAACACAAGAGAAATTCTAT |
| | | GGCCTACCATATGTTCCAAATACTGCCTGGCAGCTGCGATTCAGCCACCTCGTGGG |
| | | GTATGGTGCTAGATATTACTCTTACCTCATGTCCAGAGCGGTCGCCTCCATGGTTT |
| | | GGAAGGAGTGTTTTCTACAGGATCCTTTCAACAGGGCTGCCGGGGAGCGCTATCGC |
| | | AGGGAGATGCTGGCCCACGGTGGAGGCAGGGAGCCCATGCTCATGGTTGAAGGTAT |
| | | GCTTCAGAAGTGTCCTTCTGTTGATGACTTCGTAAGTGCCCTCGTTTCCGACTTGG |
| | | ATCTGGACTTCGAAACTTTCCTCATGGATTCTGAATAAAAGAAACACTCTACACCT |
| | | CTTAAATCAAGGTCATGTAGATAATGACTTTGTTATAAATGCTACAGCTGTGAGAG |
| | | CTTGTTTCTGATTTCATTGTTCGCTTCTGTAATTCTGAAAAACTTTAAACTGGTAG |
| | | AACTTGGAATAAATAATTTGTTTTAATTA |
| 35 | MS4A8 | AGCATGTAACCTGGCCTGCATCCAGGAAATAGAGGACTTCGGATCCTTCTAACCCT |
| | | ACCACCCAACTGGCCCCAGTACATTCATTCTCTCAGGAAAAAAAACAAGGTCCCCA |
| | | CAGCAAAGAAAAGGAATAGGATCAAGAGATACGTGGCTGCTGGCAGAGCAAGCATG |
| | | AATTCGATGACTTCAGCAGTTCCGGTGGCCAATTCTGTGTTGGTGGTGGCACCCCA |
| | | CAATGGTTATCCTGTGACCCCAGGAATTATGTCTCACGTGCCCCTGTATCCAAACA |
| | | GCCAGCCGCAAGTCCACCTAGTTCCTGGGAACCCACCTAGTTTGGTGTCGAATGTG |
| | | AATGGGCAGCCTGTGCAGAAAGCTCTGAAAGAAGGCAAAACCTTGGGGGCCATCCA |
| | | GATCATCATTGGCCTGGCTCACATCGGCCTCGGCTCCATCATGGCGACGGTTCTCG |
| | | TAGGGGAATACCTGTCTATTTCATTCTACGGAGGCTTTCCCTTCTGGGGAGGCTTG |
| | | TGGTTTATCATTTCAGGATCTCTCTCCGTGGCAGCAGAAAATCAGCCATATTCTTA |
| | | TTGCCTGCTGTCTGGCAGTTTGGGCTTGAACATCGTCAGTGCAATCTGCTCTGCAG |
| | | TTGGAGTCATACTCTTCATCACAGATCTAAGTATTCCCCACCCATATGCCTACCCC |
| | | GACTATTATCCTTACGCCTGGGGTGTGAACCCTGGAATGGCGATTTCTGGCGTGCT |
| | | GCTGGTCTTCTGCCTCCTGGAGTTTGGCATCGCATGCGCATCTTCCCACTTTGGCT |
| | | GCCAGTTGGTCTGCTGTCAATCAAGCAATGTGAGTGTCATCTATCCAAACATCTAT |
| | | GCAGCAAACCCAGTGATCACCCCAGAACCGGTGACCTCACCACCAAGTTATTCCAG |
| | | TGAGATCCAAGCAAATAAGTAAGGCTACAGATTCTGGAAGCATCTTTCACTGGGAC |
| | | CAAAAGAAGTCCTCCTCCCTTTCTGGGCTTCCATAACCCAGGTCGTTCCTGTTCTG |
| | | ACAGCTGAGGAAACGTCTCTCCCACTGTTTGTACTCTCACCTTCATTCTTCAATTC |
| | | AGTCTAGGAAACCATGCTGTTTCTCTATCAAGAAGAAGACAGAGATTTTAAACAGA |
| | | TGTTAACCAAGAGGGACTCCCTAGGGCACATGCATCAGCACATATGTGGGCATCCA |
| | | GCCTCTGGGGCCTTGGCACACACACATTCGTGTGCTCTGCTGCATGTGAGCTTGTG |
| | | GGTTAGAGGAACAAATATCTAGACATTCAATCTTCACTCTTTCAATTGTGCATTCA |
| | | TTTAATAAATAGATACTGAGCAT |
| 36 | MYO6 | GGAGACCGACTCGGGATCTGTCCGAGCAGGAAGCCAGCCTCAGCCCGGCCGCTGTC |
| | | GCCGCCCTGTCCTGGTGCCCGTCCGCGTCGTCGCCCTCTTCACTGGCCCTCATCAC |
| | | TTCTCACCGCGCCCTCCAGCTTCACCCGTACAGGTAGCCCCGCCGCCGCGCACCTG |
| | | CCTTCGCTCCCGCACCGGTGACAGTGGATAGTGGAAACAGGAGATCGTGGATCCTC |
| | | CTTCAAAAATGGAGGATGGAAAGCCCGTTTGGGCGCCACACCCTACAGATGGATTT |
| | | CAGATGGGCAATATTGTGGATATTGGCCCCGACAGCTTAACAATTGAACCCTTGAA |
| | | TCAGAAAGGCAAGACATTTTTGGCTCTCATAAACCAAGTGTTTCCTGCAGAAGAGG |
| | | ACAGTAAAAAAGATGTGGAAGATAACTGTTCACTAATGTATTTAAATGAAGCCACA |
| | | CTGCTCCATAATATCAAAGTTCGATATAGTAAAGACAGAATTTATACATATGTCGC |
| | | CAACATTCTGATTGCAGTGAATCCATACTTTGACATACCTAAAATATATTCTTCAG |
| | | AAGCAATAAAGTCATATCAAGGAAAATCTCTTGGGACAAGACCACCTCATGTCTTT |
| | | GCAATTGCTGATAAAGCTTTTCGAGACATGAAGGTGCTCAAGATGAGTCAGTCTAT |
| | | CATTGTATCTGGAGAATCAGGAGCCGGCAAAACAGAAAATACAAAATTTGTTCTAA |
| | | GATACCTGACTGAATCCTATGGAACAGGTCAAGATATTGATGACAGAATTGTTGAA |
| | | GCTAACCCACTCCTAGAAGCCTTTGGAAATGCGAAGACTGTTCGCAACAATAATAG |
| | | CAGTCGATTTGGGAAATTTGTAGAAATACATTTTAATGAAAAGAGCTCAGTTGTTG |
| | | GAGGATTTGTTTCACATTATCTCCTAGAGAAATCTAGGATCTGTGTTCAAGGCAAA |
| | | GAGGAAAGAAATTATCATATCTTTTATAGGTTGTGTGCTGGTGCTTCTGAAGATAT |
| | | TAGAGAAAAACTTCATTTGAGTTCACCAGATAATTTTCGGTATTTAAACCGAGGCT |
| | | GCACTAGATACTTTGCTAACAAAGAAACTGACAAACAGATTTTACAGAACCGCAAA |
| | | AGTCCTGAGTACCTTAAGGCAGGTTCTATGAAAGATCCTCTGCTAGATGACCATGG |
| | | TGATTTTATTAGAATGTGCACGGCTATGAAAAAAATTGGTTTGGATGATGAAGAAA |
| | | AGCTTGATCTCTTCCGGGTAGTAGCTGGCGTCCTGCACCTTGGAAATATTGATTTT |
| | | GAGGAAGCTGGCAGCACTTCAGGTGGTTGTAATCTGAAGAATAAATCTGCTCAGTC |
| | | TTTGGAATATTGTGCTGAATTACTGGGTTTGGACCAAGATGATCTTCGAGTAAGTT |
| | | TGACCACAAGAGTCATGCTAACAACAGCAGGGGGCACCAAAGGAACAGTTATAAAG |
| | | GTACCTCTGAAAGTGGAGCAAGCAAACAATGCTCGTGATGCCCTGGCAAAGACAGT |
| | | GTATAGCCATCTTTTTGATCATGTGGTAAACAGAGTAAATCAGTGTTTTCCTTTTG |
| | | AAACATCATCCTATTTTATTGGAGTCCTAGATATTGCTGGTTTTGAGTACTTTGAG |
| | | CATAACAGTTTTGAACAATTTTGCATCAACTATTGCAATGAAAAACTTCAACAATT |
| | | TTTTAATGAAAGGATTCTGAAGGAGGAACAAGAACTCTATCAAAAAGAAGGTTTAG |
| | | GTGTTAATGAAGTGCATTATGTGGATAATCAGGACTGTATAGATTTAATTGAAGCC |
| | | AAATTAGTGGGAATACTGGATATTTTGGATGAAGAAAATCGCCTTCCCCAGCCAAG |
| | | TGATCAACACTTTACATCTGCAGTTCACCAAAAGCACAAGGATCATTTTCGACTCA |
| | | CTATTCCCAGAAAATCTAAGCTGGCAGTTCATAGGAATATCAGAGACGACGAAGGC |
| | | TTCATTATCAGGCATTTTGCGGGGGCAGTGTGCTATGAAACAACCCAGTTTGTGGA |
| | | GAAAAATAATGATGCTTTACATATGTCTCTTGAATCCTTAATATGTGAATCCAGAG |
| | | ATAAGTTTATACGGGAATTATTTGAATCATCCACAAATAACAACAAAGATACTAAA |
| | | CAAAAAGCAGGAAAACTTAGCTTCATCAGCGTGGGAAACAAGTTTAAGACACAGTT |
| | | AAATTTGCTTCTGGATAAACTTCGAAGTACTGGAGCAAGCTTTATTCGTTGCATCA |
| | | AACCTAACTTAAAGATGACAAGCCACCACTTTGAAGGTGCTCAAATTCTGTCTCAG |
| | | CTTCAGTGTTCAGGGATGGTGTCTGTTTTGGACTTGATGCAGGGTGGTTACCCATC |
| | | ACGAGCTTCATTTCATGAACTCTACAACATGTACAAAAAGTATATGCCAGATAAAC |
| | | TTGCAAGATTGGATCCAAGACTATTTTGTAAGGCTTTGTTTAAAGCTTTGGGCTTA |
| | | AATGAAAATGACTACAAGTTTGGGTTAACCAAAGTATTTTTTAGACCTGGCAAGTT |
| | | TGCAGAATTTGATCAGATCATGAAGTCTGACCCTGACCACTTAGCAGAGTTGGTTA |
| | | AAAGAGTCAATCACTGGCTCACATGCAGTCGCTGGAAGAAAGTTCAGTGGTGCTCA |
| | | CTCTCAGTCATCAAATTGAAAAACAAAATAAAATATCGAGCTGAAGCCTGCATTAA |
| | | AATGCAAAAAACTATTCGAATGTGGCTTTGCAAGAGGAGACACAAACCTCGCATTG |
| | | ATGGTCTGGTTAAGGTGGGCACACTGAAAAAACGACTTGATAAATTTAATGAGGTA |
| | | GTCAGTGTGTTGAAAGATGGAAAACCCGAGATGAATAAACAGATCAAGAATCTGGA |
| | | AATTTCTATTGATACTTTGATGGCCAAAATTAAGTCCACTATGATGACGCAGGAAC |
| | | AAATCCAGAAAGAATATGATGCACTGGTTAAAAGCTCAGAGGAACTCCTCAGTGCA |
| | | TTACAGAAAAAAAAACAGCAGGAAGAGGAAGCAGAAAGGCTGAGGCGTATTCAAGA |
| | | AGAAATGGAAAAGGAAAGAAAAAGACGTGAAGAAGACGAAAAACGTCGAAGAAAGG |
| | | AAGAGGAGGAAAGGCGGATGAAACTTGAGATGGAAGCAAAGAGAAAACAAGAAGAA |
| | | GAAGAGAGAAAGAAAAGGGAAGATGATGAAAAACGCATTCAAGCTGAAGTGGAGGC |
| | | ACAGCTGGCCCGACAGAAGGAGGAGGAATCCCAACAGCAAGCAGTTCTGGAGCAGG |
| | | AGCGCAGGGACCGGGAGCTGGCCCTGAGGATTGCCCAGAGTGAAGCCGAGCTCATC |
| | | AGTGATGAGGCCCAGGCCGACCTGGCGCTGCGGAGAAATGATGGAACAAGACCCAA |
| | | AATGACACCGGAACAAATGGCCAAAGAAATGTCAGAATTTTTGAGTAGAGGTCCTG |
| | | CTGTACTAGCCACCAAAGCAGCTGCTGGTACTAAGAAATATGATCTTAGTAAATGG |
| | | AAATATGCAGAACTACGTGATACCATCAATACTTCTTGTGATATTGAGCTCCTGGC |
| | | AGCTTGCAGAGAAGAATTTCATAGGAGACTAAAAGTGTATCATGCTTGGAAATCTA |
| | | AGAACAAGAAGAGAAATACTGAAACAGAGCAACGTGCTCCAAAGTCTGTTACTGAT |
| | | TATGATTTTGCACCATTTTTGAACAATTCACCTCAGCAAAACCCAGCAGCTCAGAT |
| | | TCCTGCCAGGCAGCGGGAGATTGAAATGAACCGACAGCAACGCTTCTTCCGCATCC |
| | | CATTCATCCGCCCTGCCGACCAGTACAAAGACCCTCAGAGTAAGAAAAAAGGCTGG |
| | | TGGTATGCCCATTTTGATGGACCATGGATTGCCCGGCAAATGGAACTCCATCCTGA |
| | | CAAGCCACCCATCCTACTTGTGGCTGGTAAGGACGACATGGAGATGTGTGAGCTGA |
| | | ATCTTGAGGAGACTGGCCTGACTCGGAAGCGTGGTGCTGAGATCTTGCCAAGACAG |
| | | TTTGAAGAAATCTGGGAACGCTGTGGAGGCATCCAGTACCTTCAGAATGCGATTGA |
| | | GAGCAGACAGGCTCGGCCCACCTATGCAACAGCCATGCTGCAGAGTCTGTTAAAGT |
| | | AGATGTTGCACACCAGCCTTACAGCTGGGAGCCTTTGCCATGGTACTTAGGTAGGG |
| | | TGTGTGCCCCCAGATTTAACCATTCCATAATCATGTTAGAGTTACTTCTATAAAGT |
| | | GAACAGATTTTATTAATCACGGCTTTTGGTGAATTTGTTTAAGGTTAATTATGGTA |
| | | GCAAATTTTGGACCTAAACATTATTTTTCTGTATCCCGCTGTAATTCCCAAAACTC |
| | | TCATTATTCTCTAACTATTACACATGGGCATATTCTGATGTTTCTCATCCTTTGCC |
| | | AGAAGACTACCTTACATCCATCGTAATTGTTCTCTAGGAAAAGAGAACTTTTTTCA |
| | | AAATTCAAAATACTTTTTAAGGATGGCACAGTACCATATAACTGGAGTAATAAAAC |
| | | ATGAGCTTACATTCTTACAATAACTAAACCACTTAAAATGATCAAGGCACTAATGT |
| | | TTTGGTCTGAAAAGCTGTGTACTTTATAGACATTTTCAGACATTTTTGGAAATTTC |
| | | CATTAAAGGTGGAAAATCTATTTTTTTCCTCCTTTGCAGTGTCTTAGTTTGAATGA |
| | | AACACTTCGAAGTTCTAGAATTCTAGAAAGAGCCTTAATGTATTTGATGTATTCTG |
| | | TGATAAGAGGTACTAATAGTATCCAGCACAGATTTGCTTTTCTTTGCTAGCACAAT |
| | | GTGTGTTGCTGTCAGAATATTCTTTTTATATTCTGTGGAAAAATAAAGGAAATTCA |
| | | GATTGTTTAAATGCCTAAAAGTTTTGAGATAAGTTTTGTTTCAATTAGAAAAGGAA |
| | | ATAGGTTTTAGGTGGCATAGTGGCTTAACTGGACTGAATTCAAATATTCTTTCAAC |
| | | TTCATCTCAATAGTGATTTTTGTATCAGAATCTTGTCCAAGTIGTTTCATTGATTT |
| | | AGTAAGTGTTCTGCTTCCAACATCTTTCTTTTTAAGAAATTCCTAGTGTCTTTTTT |
| | | GGCCTTTGAGGTTTTGGTAATTGTAGACCTGTTTCATAAGCTTTGTAATTCAGAAA |
| | | TCCTTGTATTTAGTAAGTGCTTGTTTTACATAACTGATAATTTTAAAATGTTTTCT |
| | | TTGTGTGCTGTTAGTATTGATTCAAATGTCAGCAGCTTTAAGCCTAATATTTATGA |
| | | CTTTCACATTTGGAATTTAAAGACAAAAATACATCAAGGAGTTATGCTGACATAAT |
| | | TCTAAGGAGTTTTGTTGTATTTTAGAATAAAATTATAAAGTAAAATGATTCTCTGT |
| | | ACTGCTTTTTCCCCCAGTTTTTAGAGACCCTAACCTTTGAAATGAAATTCCAGTGA |
| | | TTTCTTTTTTCCCTAGAAAGATTACCTCAGTTAGGGAAGTATTTCCCAGCTGACTA |
| | | GTGTTTGTGAGCCACAGACACTGTCTTCAGAATTGCTTCTCTCATGTCTTAGTAGA |
| | | GAAATATTTATTTATTATGATACATTCAAATGATTGTCAAGTTAAATTAAATGGTT |
| | | GTGTCTGTGCTATTGAGAATGCAAATGTGATTATCTTTTGAAGGCTGTATTACTGC |
| | | ATAGCTTCACCCACCCTCGGGTCATTTCGTCCCTGTGATTGGGGACAGAAGGTGTA |
| | | GCTACTGAAGTAAATGACCTATTCTCTCTCTTCCATCTCTCGCCTTTAACTGGTGT |
| | | TTTTATTTGTGTAGGATAGTGAATGATAAGCTTTTTTCCTAACCAGTAGTGAGTAA |
| | | AGTTCTTGAACAAAATTTAGTAGCCAAATTGTTTTTTAATGACATGTCTCTTTAGT |
| | | ACAATAGTTTTGTGTATCTTTTAGATACATTAATAGGCACTAGATGGAAAATTAAA |
| | | GAGTTAAACATATTTAAATGAGAGAATCTAATGTTTCAGAAATTTGTAAGAAATGT |
| | | ATCACAGCAAAGGGTTGTTATAAGTCCTTAGTTTTTGACTCTAATAGTTAATACAA |
| | | TTATAGTTAATCTTAAGCCATAATGTTTCTAATCATGTCACACAGCTGTCCTAGAA |
| | | CTTATCTATTTAAAATAGTTTCCTGAGTTAATTTTGGCCAGCAGGGCAACTGCCCT |
| | | AATTCAGATAGATTTACAGTAACCTACGTACAGTAGATGCACATACACACAGACAC |
| | | CCCTTTGCTGGAGAAACTTAGGACCCTGTCAGCCTTTTAAAGGAAACAGCAGGAGT |
| | | GGTGTCCTAAATGATGTTCATGCAGCTGCTTTACCATGTTCACAGTCAAGCCCATG |
| | | CATGCCAGGTTAAAACTGTGGAAATCAAAAGTAAATTCACTCATATTTTAATCATT |
| | | TTAACTGAGATTTAAAATTAGAAGTTTAAACCACTATATATAAAGAACTAATCTTT |
| | | TCTTAATACCAGTTCTTTCCATAGCATATGCTTTGCAAAGGCAGCATGCATAAAAT |
| | | ATTTAAAATGAGAGGACAGAATGTTTTCACATTTGATTCAATTTTAATATAATTCC |
| | | TAATTGTGGTAACACAGTTGAGATATGTATTATGAGTTATGGGAACTAATTGAGAA |
| | | AAGGAAGTTACTCTAATCCACGTATGTTAAGAGAATATTGAGTTTTCTTAGITGTA |
| | | AAGTTGGGGAGATGGCACCTTCTCAGAGGATTGTGAAAATATGAGGAAGAAACAAA |
| | | ACAGTGCATGTAGGAGCACAGGGCCACACAAAGGCATTCTATTGTTATGCTCATTC |
| | | TGCTTCTGTAATGACTTTTCATAGGTCATTCTTGTGAACCATTTTGTTTTGCAAGC |
| | | AACCAAGGAAAGAACATCTTAAGTGGAAAATCAGTGGTGGTTGTGAACACTTAGAG |
| | | AATAGCAATCCACAGGCAAGAATAATGGTATTGTTTGTAGAGCTTTATTAATTGGA |
| | | TATTTTTTAAAAGACATTTTCATTCACAGGTCATTACTATGGTTCTCAGCGATCCA |
| | | AATATGTAGATCATTGGTTTTTTTTTTTACCTGAAGTAGCTTAAGAGTACTTGGAT |
| | | CAGTAGAATAAATATTTATTGAATCAATCAGTCAGCCAATTAATATGATGTTAGTG |
| | | ATAGACCTGCCTCCTTTTATGGAAGAGGTAACAGATCCAGAGAGGTCAAGTAATTT |
| | | AGTTGTAGACTGAAAAATATATCAAAGCCTTTGCTGCAATCATATGTAACAAAAAG |
| | | AACCAAAACAAACACTTTTTAGTGGCACCTGTGGATTTACAAAGGGTTGCCTCTCT |
| | | GTCATTCCACAACTTCAGAAGGTGTGACAGGTTTTCCCTATTTATCATTACCAATA |
| | | ATAACAAGTATTGAGAGTTTTAAAATTTCTCCCAGAAGATAAACTAACAAGGATGG |
| | | AAGGGGAGGGCAAAGGATATCTAAACATGAGAATAAGGACATGTTAGAGGGGGGGA |
| | | AACAGTTGTAACAATAAGGAAAGAGAAGAGCAACAGTGGAAGAGACAGGTTGTGTG |
| | | CCCCTAAAGATTCTGCACCCCCAGTTTGGAAACACTGATACATTTTAGGACACAGA |
| | | GCACTCCTAGATCTCTACGAAATTTTAGAATGAATAATGTGTAATTTATAGGATCA |
| | | GAACGTATGGTTATTAAAACTTGGATCAAGATATGCCCGGTGTATACATTCTTAGC |
| | | ACATAGGAATGGCACTGCCATACTGGAGAAGGTCAGCAGTAAATAGGCATTCTGTA |
| | | CATAAGCCTCATGGAAGGGTAAGATGGAGAGACTGGCAGAAGTAGCACCTACTCTG |
| | | CTGGGAGCACTTCTCTGAGTACGCTTTAGTTCAATTCAAATCACTGTATTCTTTCC |
| | | CCATTGCTAACCTAATATATGAAACAAGCTTAGCTGTCTCAGAAGTTTTTCAAGAG |
| | | ATGATCAGGAAAAATTAATGCACATTCAAAAGGAGAATCTTCAGTACAAATTTGTT |
| | | TTTTTAAAAATAGATTTAGGGCTGGGCGCGGTGGCTCACGCCTGTAATCCCAGCAC |
| | | TTTGGGAGGCCGAGGCGGGTGGATCACGAGGTCAAGACTTCGAGACCAGCCTGGCC |
| | | AACATGGTGAAACCCTGTCTCTACTAAAAATACAAAACATTAGCCAGGCGTGGTAG |
| | | TGGGTGCCTGTAATCCCTGCTACTTGGGAGGCTGAGGCAGGAGAATCACTTGAACG |
| | | CGGGAGGCAGAGGTTGCAGGGAGCCGAGATGGCGCCATTGCACTCCATCCTGGGCA |
| | | ACAAGAGCGAAATTCCATCTCAAAAAATAAAATAGATTTAGGGGGTACAAGTGCAG |
| | | TTTTGTTACATGGGTATATTGCATAGTGGTAAAATGTGGGCTTTTAGTGTACCTAA |
| | | CACCCAGAGAAGCATACATTGTGCCCAGTAGGTAATTTTTCATCCCTAAACCTTTT |
| | | CTCAGCCTCCCACTTTCTGGAGTCTCCAATGTCGGTTATTCCACTCTTTATGTCCA |
| | | TATCTACACATTCAATCCTAATTTGTACCAAGTAGCATCTCACCTTTAAATCACAG |
| | | GCTTATTAGTTGGGTGTTTTCTTTTTACTTATGAAAATTCATCTAGTCAAACTGTC |
| | | AATTAATTTTTCCTCATTTCATTAAAAGTGTATATCTAATGCTTTCTCTAAAAATT |
| | | GATGTACTGGAAATACAAATAAATAAATGCTCCCTGTGTAGAATTTC |
| 37 | CYB561A3 | TCTATGGCCAGTCTGGCCGCCATGTTGGAGCTTTCGCTGTTCGGGGCCCGGCCTGC |
| | | TCCGGGCCCTCCCAGCGTCGCCGCTAGCTGACTTAGGGCTCCCGCCCACACTGCGC |
| | | ACGGCCTTGCTGAGGGCCGGCTCGTAGTGGGAGGCTTCGCGTTCACCTGAACATTT |
| | | CCGCCTCCTCTGGATGGGGGATGAGAGGCGGGGCTGATGCATCGCAGCAGTCCTGG |
| | | ACCCTGACTTCGGACTTGGAGAAGAAACGCCGAGGAGGAACGGGAGTTAGGGGGTT |
| | | AGCAGCTTGGCGCGCTGGGAGCCGGCCACCCCTGGTCTCCGGACTTCACTTCCCAG |
| | | GAGGCCTCGCGCGCGACTGGAAGTGCTGCGAGCCTATAAGAAGGCGAGGCGGCACC |
| | | CGCCGCTCTGCTCTGGGGCGGCATTGCCAGCCGGCTGTAGGCATTCAGGGCAGTGT |
| | | CTTCTGCATCTCCTAGGAACCTCGGGAGCGGCAGCTCCGGCGCCTGGTAGCGAGAG |
| | | GCGGGTTCCGGAGATCCCGGCCTCACTTCGTCCCACTGTGGTTAGGGGTGAGTCCT |
| | | GCAAATGTTAAGTGATTTGCTCAAGGTGCCCATTTCGCAGGAATTGGAGCCCAGGC |
| | | CAGTTCTCTGAGCCTATCATTAGGGCTAAAGAGGAAGAGAAAGCAGAAGTCTCAGG |
| | | GCTGGAGACAGCTGAGGGGCATCAGCTGGTTCAAGGTGTGGAGTGTGGGCAGTGGA |
| | | GTGAAGGCCTGGTTACCTCTCCAGTGAAGATTAGCCCCATAGAGCTTACTCTATGG |
| | | GGAAGAACTTTTCTGACTCCTTCCTGTCTAGGGAGTGCGTGATCAGAATGGTGTCT |
| | | GGACGGTTCTACTTGTCCTGCCTGCTGCTGGGGTCCCTGGGCTCTATGTGCATCCT |
| | | CTTCACTATCTACTGGATGCAGTACTGGCGTGGTGGCTTTGCCTGGAATGGCAGCA |
| | | TCTACATGTTCAACTGGCACCCAGTGCTTATGGTTGCTGGCATGGTGGTATTCTAT |
| | | GGAGGTGCGTCACTGGTGTACCGCCTGCCCCAGTCGTGGGTGGGGCCCAAACTGCC |
| | | CTGGAAACTCCTCCATGCAGCGCTGCACCTGATGGCCTTCGTCCTCACTGTTGTGG |
| | | GGCTGGTTGCTGTCTTTACGTTTCACAACCATGGAAGGACTGCCAACCTCTACTCC |
| | | CTTCACAGCTGGCTGGGCATCACCACTGTCTTCCTCTTCGCCTGCCAGTGGTTCCT |
| | | GGGCTTTGCTGTCTTCCTCCTGCCCTGGGCGTCCATGTGGCTGCGCAGCCTCCTAA |
| | | AACCTATCCACGTCTTTTTTGGAGCCGCCATCCTCTCTCTGTCCATCGCATCCGTC |
| | | ATTTCGGGCATTAATGAGAAGCTTTTCTTCAGTTTGAAAAACACCACCAGGCCATA |
| | | CCACAGCCTGCCCAGTGAGGCGGTCTTTGCCAACAGCACCGGGATGCTGGTGGTGG |
| | | CCTTTGGGCTGCTGGTGCTCTACATCCTTCTGGCTTCATCTTGGAAGCGCCCAGAG |
| | | CCGGGGATCCTGACCGACAGACAGCCCCTGCTGCATGATGGGGAGTGAAGCAGCAG |
| | | GAAGGGGCTCCCAAGAGCTCCTGGTGGTGCAGCCTGTGCTCCCCTCAGAAGCTCTG |
| | | CTCTTCCCAGGGCTCCCGGCTGGTTTCAGCAGGCGACTTTCTTCCAATGCTGGGCC |
| | | CAGACTTCTTGCCTGGGTGCTGGCCTGCCCTCTCCGGCCGCTTGCTGCCTGTCTGC |
| | | TTTCCTTGGTGGCTTTGCCTGGGTGCTGGGCCTGCCCTCTCCGGCCGCTTGCTGCC |
| | | TGTCTGCTTTCCTTGGTGGCTTTGCCTGGGTGCTGGGCCTGCCTTCTCTGGCTGCT |
| | | TGCTGCCTGTCTGCTTTCCTTGGTGGCTTTGGCTTCTGCACTCCTTGGCGTCAGCC |
| | | TCTCAGGTCCTCCATTCACACGAGGTCCTCCTCGCTCTGGCCGCTCTTGCTGCTCC |
| | | TGTCTGAAGAAATCAGACTGATTTCCTCTTAAGACTCCTAGGGATGTGGTGAAGAG |
| | | CTGGGACTCAAGTGCAGTCCACGGTGTGAAACATGAGGGAGGTGAGGTGTCCGTCC |
| | | ACTTCCCCCATAAAGGTGTGCATTTCAGTTAGGCTGCCCCGCCACAGAGCAGGCTT |
| | | CATCTGCTCTGCCATCCAGCCCCATCTGGATGTGAGGTGGGGTGGAGACATCATGG |
| | | GGTGATTGCAGAAAGGGGGAGTGGCGGCCCACGCAGCTTCTGCTGAGGAGCTGACC |
| | | GCTCTGAGCTGTTCTGTTTCGTATTGCTGCTCTGTGTCTGCATGTATTGTGACCGT |
| | | GCGGCTCCACCTCTTCCAGCTGCTGCTACAGCTGAGGCCTGGATCCCGGCCTTTCC |
| | | CTGTGACTTACGTGTCTGTCACCGGCAGGCAGCCCTACAAATCCTGGTGACCTGCT |
| | | CTCCCAAGAACAGAGCCTGTCCCCAGATGTCCCAGTAGCGATGAGTAACAGAGGTG |
| | | GCTGTGGACTTCCTCTACTTCTCCTTGCTGGATCAGGGCCTTCCTGCCTCCCGCTG |
| | | GGCAGGTCTGGCCTTGCTCTCTTGGCAGGGCCCCAGCCCCTCTGACCACTCTGCAG |
| | | CTCACCATGCAGCTGATGCCAAAGTTGTGGTGTCCAGTGTGCAGCAGCCCTGGGAG |
| | | CCACTGCCACCTTCAGAGGGGTTCCTTGCTGAGACCCACATTGCTTCACCTGGCCC |
| | | CACCATGGCTGCTTGCCTGGCCCAACCTAGCGTTCTGTGCCATGCTAGAGCTTGAG |
| | | CTGTTGCTCTTCTTCAGGGGAGGAAATAGGGTGGAGAGCGGGAAGGGTCTTGCTCC |
| | | TAAGTGTTGCTGCTGTGGCTTTTTTGCCTTCTCCAAAGACGCACTGCCAGGTCCCA |
| | | AGCTTCAGACTGCTGTGCTTAGTAAGCAAGTGAGAAGCCTGGGGTTTGGAGCCCAC |
| | | CTACTCTCTGGCAGCATCAGCATCCTACTCCTGGCAACATCAGGCCAACGTCCACC |
| | | CCAGCCTCACATTGCCAGATGTTGGCAGAAGGGCTAATATTGACCGTCTTGACTGG |
| | | CTGGAGCCTTCAAAGCCACTGGGATGTCCTCCAGGCACCTGGGTCCCATGACCAGC |
| | | TCCCCATCTCCATAGGGGTAGGCATTTCACTGGTTTATGAAGCTCGAGTTTCATTA |
| | | AATATGTTAAGAATCAAAGCTGTCTTTGTTCAGGCTGCTATAACAAAAATATAATA |
| | | GCCTGGGTGGCTTAAACAAAAA |
| 38 | PDLIM5 | ACTTGTCAGCCCTTGTCTGAGGCGGAGGCAGCCCCGCGCCGCGCCGGACCCGAGCA |
| | | TATTTCATTTTCTGTCATTGGACTTTGAGCCATTAGAACCATGAGCAACTACAGTG |
| | | TGTCACTGGTTGGCCCAGCTCCTTGGGGTTTCCGGCTGCAGGGCGGTAAGGATTTC |
| | | AACATGCCTCTGACAATCTCTAGTCTAAAAGATGGCGGCAAGGCAGCCCAGGCAAA |
| | | TGTAAGAATAGGCGATGTGGTTCTCAGCATTGATGGAATAAATGCACAAGGAATGA |
| | | CTCATCTTGAAGCCCAGAATAAGATTAAGGGTTGTACAGGCTCTTTGAATATGACT |
| | | CTGCAAAGAGCATCTGCTGCACCCAAGCCTGAGCCGGTTCCTGTTCAAAAGGGAGA |
| | | ACCTAAAGAAGTAGTTAAACCTGTGCCCATTACATCTCCTGCTGTGTCCAAAGTCA |
| | | CTTCCACAAACAACATGGCCTACAATAAGGCACCACGGCCTTTTGGTTCTGTGTCT |
| | | TCACCAAAAGTCACATCCATCCCATCACCATCGTCTGCCTTCACCCCAGCCCATGC |
| | | GACCACCTCATCACATGCTTCCCCTTCACCCGTGGCTGCCGTCACTCCTCCCCTGT |
| | | TCGCTGCATCTGGACTGCATGCTAATGCCAATCTTAGTGCTGACCAGTCTCCATCT |
| | | GCACTGAGCGCTGGTAAAACTGCAGTTAATGTCCCACGGCAGCCCACAGTCACCAG |
| | | CGTGTGTTCCGAGACTTCTCAGGAGCTAGCAGAGGGACAGAGAAGAGGATCCCAGG |
| | | GTGACAGTAAACAGCAAAATGGCCCACCAAGAAAACACATTGTGGAGCGCTATACA |
| | | GAGTTTTATCATGTACCCACTCACAGTGATGCCAGCAAGAAGAGACTGATTGAGGA |
| | | TACTGAAGACTGGCGTCCAAGGACTGGAACAACTCAGTCTCGCTCTTTCCGAATCC |
| | | TTGCCCAGATCACTGGGACTGAACATTTGAAAGAATCTGAAGCCGATAATACAAAG |
| | | AAGGCAAATAACTCTCAGGAGCCTTCTCCGCAGTTGGCTTCCTCGGTAGCTTCCAC |
| | | ACGGAGCATGCCCGAGAGCCTGGACAGCCCAACCTCTGGCAGACCAGGGGTTACCA |
| | | GCCTCACAGCTGCAGCTGCCTTCAAGCCTGTAGGATCCACTGGCGTCATCAAGTCA |
| | | CCAAGCTGGCAACGGCCAAACCAAGGAGTACCTTCCACTGGAAGAATCTCAAACAG |
| | | CGCTACTTACTCAGGATCAGTGGCACCAGCCAACTCAGCTTTGGGACAAACCCAGC |
| | | CAAGTGACCAGGACACTTTAGTGCAAAGAGCTGAGCACATTCCAGCAGGGAAACGA |
| | | ACTCCGATGTGCGCCCATTGTAACCAGGTCATCAGAGGACCATTCTTAGTGGCACT |
| | | GGGGAAATCTTGGCACCCAGAAGAATTCAACTGCGCTCACTGCAAAAATACAATGG |
| | | CCTACATTGGATTTGTAGAGGAGAAAGGAGCCCTGTATTGTGAGCTGTGCTATGAG |
| | | AAATTCTTTGCCCCTGAATGTGGTCGATGCCAAAGGAAGATCCTTGGAGAAGTCAT |
| | | CAGTGCGTTGAAACAAACTTGGCATGTTTCCTGTTTTGTGTGTGTAGCCTGTGGAA |
| | | AGCCCATTCGGAACAATGTTTTTCACTTGGAGGATGGTGAACCCTACTGTGAGACT |
| | | GATTATTATGCCCTCTTTGGTACTATATGCCATGGATGTGAATTTCCCATAGAAGC |
| | | TGGTGACATGTTCCTGGAAGCTCTGGGCTACACCTGGCATGACACTTGCTTTGTAT |
| | | GCTCAGTGTGTTGTGAAAGTTTGGAAGGTCAGACCTTTTTCTCCAAGAAGGACAAG |
| | | CCCCTGTGTAAGAAACATGCTCATTCTGTGAATTTTTGAAAGTCAACAGTTCAGGA |
| | | GAAGAGAAGGAATTTGAAGAGAAAAAGGAAAATTAAAATTACTAATTAATTTTTAG |
| | | ATTCAATATTTATATGGAGTTTTGAAAAATAATAGTGGCCCTGAAGGAATAAATTC |
| | | CAGCTTTAAAAACCAAGTCTGAGGAAATATTTGGCTTCATAAAGTAAAGAGACGGT |
| | | TTGGCATTTATTATTACTTTTTCCTGTATTTTATGCCCATAAAATAAGCTTTATAA |
| | | AAACCAATTTCCTGATGGACTATTAAATTCATCTTAGAATAAATTAGTGAAGAATT |
| | | TAATTTTAGAATAAATAATCCAATCTGAAATAATTATACCTTCTTTCCTTGTTAGG |
| | | TAGTTATGAGTAAATCTGCAAAAGGCAATGAAAATGCCTTAAATTTTATCAATAAC |
| | | AGAATTATTGTATTTAAAAAAAAACTAATACTTATCTTTAAAATAGTAAATAGGAT |
| | | TTTAAACAGAGAATTTTATCAGTAATAGGTGTCAGTTTTTAAAAAATTGCTTGTAG |
| | | GCTGAGCGCGGTGGCTCACGCCTGTAATCCCAGCACTTTGGGAGGCCAAGGTGGGT |
| | | GGACCACATGAGGTCAGGAGTTTGAGATCAGCCTGGCCAACATGGTGAAACCCCAT |
| | | CTCTACTAAAAATACAAAAATTAGCCGGACGCAGTGGCACGCGCCTGTAATCCCAG |
| | | CTACTCAAGAGGCTGAGGCACGAGAATCACTTGAACCCGGGAGGGAGAGGTTGCAG |
| | | TGAGCCAAGATCGTACCACTGCACTCCAGCCTGGGTGACAGAGTGAGACTCTGTCT |
| | | CCAAAAAAAAACTTTGCTTGTATATTATTTTTGCCTTACAGTGGATCATTCTAGTA |
| | | GGAAAGGACAATAAGATTTTTTATCAAAATGTGTCATGCCAGTAAGAGATGTTATA |
| | | TTCTTTTCTCATTTCTTCCCCACCCAAAAATAAGCTACCATATAGCTTATAAGTCT |
| | | CAAATTTTTGCCTTTTACTAAAATGTGATTGTTTCTATTCATTGTGTATGCTTCAT |
| | | CACCTATATTAGGCAAATTCCATTTTTTTCCCTTGTGCTAAGGTAAAGATTTAATT |
| | | AAATAATTTTGGCCTCTCATAGTTTTCTCTCTCTTTAAAGAGAATAAATAGAGGGC |
| | | CAGGTGTGGTGGCTCACGCCTGTGATCCCAGCACTTTGGGAGGCCAAGACGGGCGG |
| | | ATCATGAGGTCAAGAGATCAAGATCATCCTGGCCAACATGGTGAAACCCTGTCTCT |
| | | ACTAAAAATACAAAAATGAGCTGGGCATGGTGGGGCGTGCCTGTAGTCCCATGTAC |
| | | TTGGGAGGCTGAGGCAGGAAAATTCTTGAACCCAGGAGACGGAAGTTGCAGTGAGC |
| | | TGAGATCACACCACTGCACTCCAGCCTGGTGACAGAGCAAGACTCCGGCTCTTAAA |
| | | AAAAAAAAAAAAAAAAAAAAAAGAGAGAGAGAGAATAAATAGAAAAGAATGTGGCT |
| | | GGGAATTGTGAATCAGAAGATTATACCCCCCAATTGTTTTTCAATCCCCTTTTCTC |
| | | AAATAATAAATTAGTTAAATCAGTTTCTGAGTTATGCCACTGGCTGATGAAGAGTT |
| | | GAGAGGTCTCTTTGCAGAATGATCTTTTTGTTTCGTTTTGTTTCTTCTTCTGCATT |
| | | TAAAAATTAAAAGATTGGTTTGAGGATGTGATGAAATTGAGACTTTTTGTGGTTTT |
| | | CTCTCAATAATAAGTGAACCAATTTCAAATGTGATCACAAAGTTTGGAAAGCTTTT |
| | | ATTCACAGAGGTTGGGTAGTGTTGGGAGGGGAGTTTAATTACTCAGATTGGCCTGT |
| | | TATTTGATTTCCTCCTTTGGGAAAAGAATTATGTAGATACCACATGGAGACAGGGA |
| | | AACAATTGTGGTAAAACTGTGGATCCTGTTGCTATTTGCCCAGTGAGAAAACAGAT |
| | | TCTGGTATTTGATTTGGTTTTTCTCTTTGTTTCCAGAATGGATGAAAGTCCATGAA |
| | | CCTCCTAAGTTATAATTTAAATTTGTTTGGGGCAAGGTGATTTTATAGTCGAGACA |
| | | GAGCCCTAGGTCCTTCCTGCCCCATCACTCACTTACGACATCACTTCCATTGTGTG |
| | | CATGTTTGTTATAGAGGAGGTTTTAGGCTACAATATTTGTTTAACCTCCCTAAGAA |
| | | CTTTCAAGGCATCTGTCCTGAAAGCTGTTAATTTATGGTCTAGCAGATTTATATTA |
| | | TATGCAGATAATAATTAACTGGGGATAAAAGAATGGCAAGGGGTGACACAAAGTAG |
| | | CAAACTGAATACTTCTCCAATAGCAACCCCAAGCTACCTCCTCACCCTGCATCTTG |
| | | GAGGGAGGCAGGAAATTTCTTTTGAAATAAAGTGCTGGAGCTGAATTCTGCATTAT |
| | | TTATCGTTGCTGCTGAAACCACCTATAAAAGACTTGCTGGCTAATGTGCATTGTCA |
| | | TATAATGTACACTGTCACATCTTTACAGTCTTGTATGTTATAGAATACAAAATAAG |
| | | TTGATGGTTTTGTTTGGTGTGAGCTTTTTGTTTGTTTGTTTAGTTTTGCCTTCATA |
| | | GGTTATATGCCAAGATAGTATTTGATAAGTCAATGACATTTGGATGTTTTCTTCAA |
| | | AGAATTTTATTTGACCCAGATTTCTTATAAAGTTATCTTACATTAAGGATGTCATT |
| | | TTCATCAGACCTTCTTTCTACATATTATTCATGAAGCATAATGTTGCATTTCTCCA |
| | | AATTTTATGCCTGAAAGGGTAGTGTTGCTTCCTAAGGTATCATGTTGTCTTTGTGC |
| | | TTTGTCCATCTCTTCCGTGGCGAAGCTTTATATCTGTTCCTAAAACAGTTAATCCT |
| | | GTGAAATAAATATTGAACATAATCCAGAAGAATCTCTCTGTTTCCCTTGGGGAATG |
| | | CCATATTTAATTCACCAGCAGTAATCCTTTAATAACTGGCAGAGCACTTTATTCTT |
| | | CTGGTGAGCTCCCTGAATATTTATTTTTCTGATTATAAATTTTCTATATTAGTAGC |
| | | ATTTTTTAATTATTACTTCTTCACTATAGAGCATTTACTTTTAGTCTCTAGATGTA |
| | | TATTTTGGAATGCTGTACTTGGCATAACATAGATTAAAATCATAATGCATGACTAA |
| | | AAACTCCTTGGATTTATTTCCCATTTTAAAATTTTTAGCGGTAAGTTCAGATTTAT |
| | | AATCTTTCTCTAGACTTCCATGGTCTGAATGTTGCCTGCTGAAGTAGCAACCTAAA |
| | | AAGTATCCCCTGCTTATGCTTGTCCAGTTGGCCCTCCATGTCCATAGGCTTCGCAT |
| | | CTGTGATTCAGCCCACTGTGGGTCAAAAATATTTGGGGAAAAAAATGGATGGTTGC |
| | | GCCTTTGCTGAACATGTACAAACTTTTTTTTGTCATTAAACAATATAGTATAACAA |
| | | CTATTTACAAAGCATTTACATTGTATTAGCTATTATAGGTAATCTAGAGATGATTT |
| | | AAAGTGTATGGTAGGATGTGCATAGGTTATATGCAAATACTACACCATTTTCTATA |
| | | AGGGACTTGAACATCATGGACTTTAGTATCCTAGGGGGTTCTTGGAACCCATCACC |
| | | CATAGGGGCACCATAGGACAACTATAGTACCGTGTTTATTTCCTATTAATTCAGGT |
| | | TCCGTTTAGAGTCTAAAACTAAAACCTAATCATTTAGTCACAGTGTAAAAACAAAT |
| | | GGAAATAACAGCTCAAATCTTCAAAATATTACTATAGCATTATGTTTAAAATAATC |
| | | TACAACAAAAATGTACCATTTTCAAGCAGTACTACATTAGGAGCCCTTTTATAGAA |
| | | AATAATTTCTTCTTTACCCCCGTTCCAGTGTGAATCTAGTATTCTGTTAACATTTG |
| | | TGTGGCATTTGGAGTTTGTCATCCCCATTGAAGGGAGAGCCTTCTCAGACATGAAG |
| | | CAAGGGAAACATACTGAATAGTTTTACACAAATTTGATCTGGCTTCCATTTGTCCC |
| | | CCTCATTTCCCAAATGTTTAAATGTATTGGATTTGGATTCTCAATGTATAAGTTGC |
| | | CTTATCTGTTAATGTCTATCTTCTGTCTCTTTAATTTTGTATATCTGCTGTTTTGC |
| | | TTTTGGATACATTTTCTAATTAGAAGTCACATGATAAATATAATCAGTATAGTAAT |
| | | AATACCATAATGTGCACATACTCAATAAATAAATGACTGCATTGTTGTAAA |
| 39 | PEX10 | CTCGCCCGTCTGGGCGTGGGCGTGGCCGGCGTGGCTGCTCGGGACCACCCGAACCC |
| | | GCGGCCATGGCCCCGGCCGCCGCCAGCCCCCCGGAGGTGATCCGCGCGGCGCAGAA |
| | | GGACGAGTACTACCGCGGTGGGCTGCGGAGCGCGGCGGGCGGCGCCCTGCACAGCC |
| | | TGGCGGGTGCGAGGAAGTGGCTGGAGTGGAGGAAGGAGGTTGAGCTGCTCTCAGAT |
| | | GTGGCCTACTTTGGCCTCACCACACTTGCAGGCTACCAGACCCTGGGGGAGGAGTA |
| | | CGTCAGCATCATCCAGGTGGACCCATCGCGGATACATGTGCCCTCCTCGCTGCGCC |
| | | GTGGCGTGCTGGTGACACTGCATGCCGTCCTGCCCTACCTGCTGGACAAGGCCCTG |
| | | CTCCCCCTGGAGCAGGAGCTGCAGGCTGACCCCGACAGTGGGCGACCCTTGCAGGG |
| | | GAGCCTGGGGCCAGGTGGGCGTGGCTGCTCAGGGGCGCGGCGCTGGATGCGTCACC |
| | | ACACGGCCACCCTGACTGAGCAGCAGAGGAGGGCGCTGCTGCGGGCGGTCTTCGTC |
| | | CTCAGACAGGGCCTCGCCTGCCTCCAGCGGCTACATGTTGCCTGGTTTTACATCCA |
| | | CGGTGTCTTCTACCACCTGGCCAAGAGGCTCACGGGGATCACGTACCTCCGTGTCC |
| | | GCAGCCTGCCCGGAGAGGACCTGAGGGCCCGTGTTAGCTACAGGCTGCTGGGGGTC |
| | | ATCTCACTGCTGCACCTGGTGCTGTCCATGGGGCTGCAGCTGTACGGTTTCAGGCA |
| | | GCGGCAGCGAGCCAGGAAGGAGTGGAGGCTGCACCGCGGCCTGTCTCACCGCAGGG |
| | | CCTCCTTGGAGGAGAGAGCCGTTTCCAGAAACCCCCTGTGCACCCTGTGCCTGGAG |
| | | GAGCGCAGGCACCCAACAGCCACGCCCTGCGGCCACCTGTTCTGCTGGGAGTGCAT |
| | | CACCGCGTGGTGCAGCAGCAAGGCGGAGTGTCCCCTCTGCCGGGAGAAGTTCCCTC |
| | | CCCAGAAGCTCATCTACCTTCGGCACTACCGCTGAGCCGGCGCCCGGGTGGGCCTG |
| | | GACACAGATGACCTCTACGGGAGTCTGAACGCCAAGATTTAGTCTCAGGATTAACC |
| | | TTGCTTGCACAGAAGTTAGAACACTCTCAGTTTTTTGTCATGTAAGATACTAACCT |
| | | AGCCACCCTGGGAGAGAACAGAAAGCTGTCCCTGGCTGCGCTTTCTCAGCCCTGGG |
| | | AGGGGCGCCTGAACCCAGAACATTTCCCTAACCCCAACCTGGTAGGACTCAGCCAC |
| | | TTCTTCAGGAATTTCACTTATTTGGACGGGATTTTAGGTTTCCCTCCCTTCCCCAA |
| | | ACCATACAGTTGAGAAGTAATTCAGAAGTAGGCCAGAAGACACTTTATTCGTTTAT |
| | | ATTGTGAGAAAACAGCCCCATCAGGCTTGTGTTAAGGCAATGGACTGAATGAGTGC |
| | | GTGCTGGGTGGGGTGGGGCACGGAGGCTGGCGGGTTGCTTCAGCCAGTGCAGTGAG |
| | | AACAGCAGCCCCACGGCCCCATGGGAGGCGGCGCTGCTCTCCCCGAGGGCGGCTGG |
| | | GCAGAGCACATCCCCCAGGACTTGATGACCACACGGGGCAGAGAGAAACCAACCAA |
| | | GGCCAGCACCTCCGTCGGAAGCATTTGGCACACACACCTTCAATACACGTCAAGGT |
| | | CGCTTCCAGTTTTAGAAAACAGAAATCTGCATCTCAGCCTGAGACGCACAGAGAGG |
| | | TCTCTTCCTGACCCAGACGCACTCACGAGCCAGGTCCTGGGGGTATGGGGGCTGCC |
| | | AGGGGCGCCCGAGCCCTCTCCTGGGGGGCCTGCTGGGCAGGCGACCTGCTGACCCA |
| | | CGGTCACTGCTGTGTTCAGCCCCTCAGCTCGGCCCCAGCCTATTTCCCGCCTCCAT |
| | | TTGATGTTTCCAGGTTTTCAAAACTGCATTTAACCTGCGCCAGAGAGTTCACCGTA |
| | | GGCATCTTTAATAAACTAACTCCAGCAAAATGTGGGTACGTTACTAACACAAGATG |
| | | GAACTTGAGATTTCCTGAAGCGCAGTGTTAAAATGCCTCGAGGGCGTGTGCCACAT |
| | | TTCCCTGCTGACGTAAAACACCTGGCCAGCAGGACCGGCTTCCGGCAAAGACCCGC |
| | | GCAAACCATGTCTTGCTGTTTGACTTACTTAGGAATGAGGATGACCCTGTATTCCT |
| | | GGGTTAGTCAAGGTCAGCTTGCCAGAGACGGAGGAGGGCGGGGGGGCCGTGACCCT |
| | | CTAGACTGGGAGGACCACCAGGAGAGGCACTGGGGGCTGCGGCCGGGTGGGCAGAT |
| | | GCCGCCGTGGCTCCATCCATCAGCACCCAGTCCTTTGCTTCACTGTGCTCTGGCCA |
| | | AGCGCGTGCGACTTCCTGTCACACCTTAGGCCTGAGCGCTTATTAAGAAAACAGTA |
| | | TGAAAAATGACAAACACTCCTGTCCACAGTCTGAGAATCCCAAGTGTCTCAACACG |
| | | GTACCAGAAGTACCACCTGGGAGCAGCACTGGCTATCTTGGGTCAGGGACACACAA |
| | | GCTTGAACGTGGCCTCTTAGAGGAGCTCGATGTCAGATATGTAAACACTGGTACAA |
| | | GAAAATCCAGACACTAAGCATTCACCGTGTCAATTCTGAGACAGGAAACACCGCCC |
| | | AGAGTGAGGAGCCTGCGCAGCCCGGGGCCCAGGAGGAGAGGCCTTCACTCGCATGT |
| | | CAGTGTCTCATCTGGGACCACAGCTGTGATGTTCTGCTGTGCAATATGGGTCACCT |
| | | TTCGAAAAGGCCAAACAAAAGGTGGGCTTTCCTGTGTCCAGCGACTGCCATGTTTT |
| | | AGGTAAAGACTTTAGTAAAGGTGGAGGTAAAATCA |
| 40 | PLA1A | ATACCAGCTCTGAGATTTCCAGCTCAGCGATGCCCCCAGGTCCCTGGGAGAGCTGC |
| | | TTCTGGGTGGGGGGCCTCATTTTGTGGCTCAGCGTTGGAAGTTCAGGGGATGCACC |
| | | TCCTACCCCACAGCCAAAGTGCGCTGACTTCCAGAGCGCCAACCTTTTTGAAGGCA |
| | | CCGATCTCAAAGTCCAGTTTCTCCTCTTTGTCCCTTCGAATCCTAGCTGTGGGCAG |
| | | CTAGTAGAAGGAAGCAGTGACCTCCAAAACTCTGGGTTCAATGCCACTCTGGGAAC |
| | | CAAACTAATTATCCATGGATTCAGGGTTTTAGGAACAAAGCCTTCCTGGATTGACA |
| | | CATTTATTAGAACCCTTCTGCGTGCAACGAATGCTAATGTGATTGCCGTGGACTGG |
| | | ATTTATGGGTCTACAGGAGTCTACTTCTCAGCTGTGAAAAATGTGATTAAGTTGAG |
| | | CCTCGAGATCTCCCTTTTCCTCAATAAACTCCTGGTGCTGGGTGTGTCGGAATCCT |
| | | CAATCCACATCATTGGTGTTAGCCTGGGGGCCCACGTTGGGGGCATGGTGGGACAG |
| | | CTCTTCGGAGGCCAGCTGGGACAGATCACAGGCCTGGACCCCGCTGGACCTGAGTA |
| | | CACCAGGGCCAGTGTGGAAGAGCGCTTGGATGCTGGAGATGCCCTCTTCGTGGAAG |
| | | CCATCCACACAGACACCGACAATTTGGGTATTCGGATTCCCGTTGGACATGTGGAC |
| | | TACTTCGTCAACGGAGGCCAAGACCAACCTGGCTGCCCCACCTTCTTTTACGCAGG |
| | | TTATAGTTATCTGATCTGTGATCACATGAGGGCTGTGCACCTCTACATCAGCGCCC |
| | | TGGAGAATTCCTGTCCACTGATGGCCTTTCCCTGTGCCAGCTACAAGGCCTTCCTT |
| | | GCTGGACGCTGTCTGGATTGCTTTAACCCTTTTCTGCTTTCCTGCCCAAGGATAGG |
| | | ACTGGTGGAACAAGGTGGTGTCAAGATAGAGCCGCTCCCCAAGGAAGTGAAAGTCT |
| | | ACCTCCTGACTACTTCCAGTGCTCCGTACTGCATGCATCACAGCCTCGTGGAGTTT |
| | | CACTTGAAGGAACTGAGAAACAAGGACACCAACATCGAGGTTACCTTCCTTAGCAG |
| | | TAACATCACCTCTTCATCTAAGATCACCATACCTAAGCAGCAACGCTATGGGAAAG |
| | | GAATCATAGCCCATGCCACCCCACAATGCCAGATAAACCAAGTGAAATTCAAGTTT |
| | | CAGTCTTCCAACCGAGTTTGGAAAAAAGACCGGACTACCATTATTGGGAAGTTCTG |
| | | CACTGCCCTTTTGCCTGTCAATGACAGAGAAAAGATGGTCTGCTTACCTGAACCAG |
| | | TGAACTTACAAGCAAGTGTGACTGTTTCCTGTGACCTGAAGATAGCCTGTGTGTAG |
| | | TTTAACCTGGGCAGGACACATCTCCCTGCATTTTTTTTTTTTTTTTGAGAGAGAGG |
| | | TGTGATGAGGGATGTGTGTGTGCAGCTTATTGTAGACCATTACTACTAAGGAGAAA |
| | | AGCAAAGCTCTTTCTTATTTTCCTCATAATCAGCTACCCTGGAGGGGAGGGAGAAC |
| | | TCATTTTACAGAACTTGGTTTCCTTTGCCGATCTTATGTACATACCCATTTTAGCT |
| | | TTCCCATGCATACTTAACTGCACTTGCTTTATCTCCTTGGGCATTCGTACTTAGGA |
| | | TTCAATAGAAACATGTACAGGGTAAACAATTTTTTAAAAATAAAACTTCATGGAGT |
| | | ATCTGAA |
| 41 | PLA2G7 | AGTGTATTCAGAGAACACGGTGAAACAAGGAAAACCGGCCTGACTGGGGGGTGAAT |
| | | TCAGCAGGGAGTAAATCTGATCGGCATCAGGTCTGCGGAAAGGAGCTGGAGACTAA |
| | | GCTGAAACTGCTGCTCAGCTCCCAAGATGGTGCCACCCAAATTGCATGTGCTTTTC |
| | | TGCCTCTGCGGCTGCCTGGCTGTGGTTTATCCTTTTGACTGGCAATACATAAATCC |
| | | TGTTGCCCATATGAAATCATCAGCATGGGTCAACAAAATACAAGTACTGATGGCTG |
| | | CTGCAAGCTTTGGCCAAACTAAAATCCCCCGGGGAAATGGGCCTTATTCCGTTGGT |
| | | TGTACAGACTTAATGTTTGATCACACTAATAAGGGCACCTTCTTGCGTTTATATTA |
| | | TCCATCCCAAGATAATGATCGCCTTGACACCCTTTGGATCCCAAATAAAGAATATT |
| | | TTTGGGGTCTTAGCAAATTTCTTGGAACACACTGGCTTATGGGCAACATTTTGAGG |
| | | TTACTCTTTGGTTCAATGACAACTCCTGCAAACTGGAATTCCCCTCTGAGGCCTGG |
| | | TGAAAAATATCCACTTGTTGTTTTTTCTCATGGTCTTGGGGCATTCAGGACACTTT |
| | | ATTCTGCTATTGGCATTGACCTGGCATCTCATGGGTTTATAGTTGCTGCTGTAGAA |
| | | CACAGAGATAGATCTGCATCTGCAACTTACTATTTCAAGGACCAATCTGCTGCAGA |
| | | AATAGGGGACAAGTCTTGGCTCTACCTTAGAACCCTGAAACAAGAGGAGGAGACAC |
| | | ATATACGAAATGAGCAGGTACGGCAAAGAGCAAAAGAATGTTCCCAAGCTCTCAGT |
| | | CTGATTCTTGACATTGATCATGGAAAGCCAGTGAAGAATGCATTAGATTTAAAGTT |
| | | TGATATGGAACAACTGAAGGACTCTATTGATAGGGAAAAAATAGCAGTAATTGGAC |
| | | ATTCTTTTGGTGGAGCAACGGTTATTCAGACTCTTAGTGAAGATCAGAGATTCAGA |
| | | TGTGGTATTGCCCTGGATGCATGGATGTTTCCACTGGGTGATGAAGTATATTCCAG |
| | | AATTCCTCAGCCCCTCTTTTTTATCAACTCTGAATATTTCCAATATCCTGCTAATA |
| | | TCATAAAAATGAAAAAATGCTACTCACCTGATAAAGAAAGAAAGATGATTACAATC |
| | | AGGGGTTCAGTCCACCAGAATTTTGCTGACTTCACTTTTGCAACTGGCAAAATAAT |
| | | TGGACACATGCTCAAATTAAAGGGAGACATAGATTCAAATGTAGCTATTGATCTTA |
| | | GCAACAAAGCTTCATTAGCATTCTTACAAAAGCATTTAGGACTTCATAAAGATTTT |
| | | GATCAGTGGGACTGCTTGATTGAAGGAGATGATGAGAATCTTATTCCAGGGACCAA |
| | | CATTAACACAACCAATCAACACATCATGTTACAGAACTCTTCAGGAATAGAGAAAT |
| | | ACAATTAGGATTAAAATAGGTTTTTTAAAAGTCTTGTTTCAAAACTGTCTAAAATT |
| | | ATGTGTGTGTGTGTGTGTGTGTGTGTGTGTGTGAGAGAGAGAGAGAGAGAGAGAGA |
| | | GAGAGAGAGAGAGAATTTTAATGTATTTTCCCAAAGGACTCATATTTTAAAATGTA |
| | | GGCTATACTGTAATCGTGATTGAAGCTTGGACTAAGAATTTTTTCCCTTTAGATGT |
| | | AAAGAAAGAATACAGTATACAATATTCATATCAGCCTAAATTTTAATTTTAAAGAT |
| | | GATTCCTTTTCAGTGTCGAAGTTAAAAACTGTTTTTACATTACTTTGACAGACAAG |
| | | TAGATTAAAACAGGCAAAATCCCAGTGAAAA |
| 42 | PRCAT47 | TTTCTTTTTGTTTGCTGCCTTCCGTAGAAGATGTGGCTTGCTCATGCTTGACTTCT |
| | | GCCATGGTTGTGAGGCCTCCCCAGCCATGTGGAACTGTTTTCAGGTGCTGGTTCCA |
| | | TGGCTCTTCCTGAGCCGAAAATAAGGAAACTCCATAGACCTTGTCCACTGGAACTC |
| | | GTTCCCATCTACCCTCCACTCTATCCAGCCCCATGAGGACAAGGAACATGATTGGT |
| | | TTTGCTCACTGCCGTATCTTCCGTACCTAGTACGTAACAGGACATCAATAAATATT |
| | | AGTTGAATGGAAGATTAAATCAACAAAATGGGTGATGGATCTCTGCAGTAAGTGGA |
| | | AGAGTTCTTCATGGCCCCCAAGGTTATATCCATCTAGAACTTCAGCACGTAATTTC |
| | | ATCTGGAAATAGTGCCTTTGCGGATATAAGTTAGGTAAAACTGAAGATGAGATCAT |
| | | ACTGGATTAGGATGGGATCTAAATCCAATGAAAATGTCTTCATAAAAAACAGGAAA |
| | | GAACCCATAGAAACACAAGGAAGAAGGTCATGTGAAGATGGAGGCAGAGATTGGAG |
| | | GGATGCAGCCACCGGCCCAGGAATGCCAGCAGCCACCCAGAAGCTGGAAGGAAATG |
| | | AGGGATTCTCTCCTAGAACCTTTAGAGAGAACATGGTCCTGTGAACAGCTTGATTT |
| | | TGGACTTGCCCATAGCTTGTATACTCTTACTTTGGATACAATTTTATCCAAACTTG |
| | | GCTAAACAGTTTCTCAGCCTATGGAAAATTTAAAATGGAGAAGATTCAACTCGATT |
| | | CTTACAGATTCAAAGCAAGAAAATGATGGGAACATAGGAGGAGACCAAGAAAGCCT |
| | | ATAAAAAGCAAAAATATGAAGTGAACATTGTGGTAGCTTTAAGATGTTTAGTGTAG |
| | | CTGCAGGCACCCTATACACATGAAAACCCCCAAGGGGAATCCCCATATCACAGTGT |
| | | AGTGTGATATTTGACATTCGTGATCATCTAGAGATGTACAGAAAAGGTGAATCTGT |
| | | GTTCTGTATATTCTGCCTAAGGCAAAGAAATGTTTAGCTCTCTTTAAAATAGTTCC |
| | | ATAATTTTTTCTAAAAAGCTTTGCTTGAAAACTGTAAGCTTCCCATATCTGGAGCA |
| | | TTTCACTTTAAATATTTGGATAAATATGTTATCTTCTTACTTGGACATTTCATGTG |
| | | TTTAGGGATTGTCTTCTAAATTCTTCCTAATTCATATAGCTGCTAACACTTCCCGC |
| | | AGAGCTAAACCATTACAGATATGAAATAAAGACCTATTGATTTGACTTACTTTTAC |
| | | TTGTAAAACCTTCTGAGTGTTATAACCTCATTTAATCTTTCAGCATTTACAGTTTC |
| | | AAGAGTTTGTGTCACAATTAGAAGAATTCAGCTGCACCTCCAAGTGACAGCAGTTG |
| | | CTCTGGTTGGTGGGTGATCTCAGGAGGCTTGAGAATTGCTTTGTCTGTGAGGGAAG |
| | | TAAGACATTTTCAGAGCCCCACTTTAGAAGGTGTGAACTGGCTAGATAATGAACCC |
| | | CAGGGCTAACGCTGCTATAGCAGTGGGAAGGAGGTGATGGGTTTTCAGTTTGGACC |
| | | TCAAACATCAATACCCTCCTGGTACGGGGAGGAACAGAGTCCCTCCTTTACTTCTC |
| | | CATTAGAAAGAATGAGATGGCAAGACAATGAAACAGGCAAAGTGAACAGAGATGCA |
| | | AGACAAAATTCAGGTGAGAGAGCCAGAGCATCACTCAGCCATTCCTGACATGTAAA |
| | | ACAGGCAACTAGAAATTTGCAGAAAGGAAGCGAAGTCTCCATAAAGATGTTTTTAA |
| | | AGTGAGCTTGAAGTATTTGGAGACAATTCAGTGTTACATAAAATCTGCAAATCTCT |
| | | GGATAAAGAAGCAGAGATCCCAGCATGGGACAAATGGAGCCTCAAAAGTGGGAAGA |
| | | AGACAGAGAAGACCAGGGCAGAATGCATCTCTTCCTTTCTCTTGGCTTTCCTGGAT |
| | | AAGGACTGCATCATTCCTGTGGAAGGACAGGCCATCAGCTCCGAAACACTGTATGT |
| | | ATTTTCCAGTATATACTGCTAGCTGTGTGATGTTGGGAAAATTTGTTACCCTGTCT |
| | | AACCCCCACTTCCCTCATCTGTAAAATGGAAATAATGATAGTACCTACCTATCTCA |
| | | TAGGTGGCAACTACAAGGGGCAGCACACTCAGGGAATTAAGGAAGTTTCAGTGAAC |
| | | ATCAACTTTATGAACACAGTGTTCATAAAGGCAGGTCAGTGCAGTGGTTTGGGAGC |
| | | CAGGAGAAGCACGTGGGCCGGAGTGTGCCTGCAGGAGACAAGGTCAGAGATGTTGC |
| | | TAATAATGGAGAATAAAGGATGCATTCTCATTACTGAAAAAAAAAAA |
| 43 | SPINK1 | ATGAAGGTAACAGGCATCTTTCTTCTCAGTGCCTTGGCCCTGTTGAGTCTATCTGG |
| | | TAACACTGGAGCTGACTCCCTGGGAAGAGAGGCCAAATGTTACAATGAACTTAATG |
| | | GATGCACCAAGATATATGACCCTGTCTGTGGGACTGATGGAAATACTTATCCCAAT |
| | | GAATGCGTGTTATGTTTTGAAAATCGGAAACGCCAGACTTCTATCCTCATTCAAAA |
| | | ATCTGGGCCTTGCTGA |
| 44 | TDO2 | AGGTCAATGATAGCATCTGCCTAGAGTCAAACCTCCGTGCTTCTCAGACAGTGCCT |
| | | TTTCACCATGAGTGGGTGCCCATTTTTAGGAAACAACTTTGGATATACTTTTAAAA |
| | | AACTCCCCGTAGAAGGCAGCGAAGAAGACAAATCACAAACTGGTGTGAATAGAGCC |
| | | AGCAAAGGAGGTCTTATCTATGGGAACTACCTGCATTTGGAAAAAGTTTTGAATGC |
| | | ACAAGAACTGCAAAGTGAAACAAAAGGAAATAAAATCCATGATGAACATCTTTTTA |
| | | TCATAACTCATCAAGCTTATGAACTCTGGTTTAAGCAAATCCTCTGGGAGTTGGAT |
| | | TCTGTTCGAGAGATCTTTCAGAATGGCCATGTCAGAGATGAAAGGAACATGCTTAA |
| | | GGTTGTTTCTCGGATGCACCGAGTGTCAGTGATCCTGAAACTGCTGGTGCAGCAGT |
| | | TTTCCATTCTGGAGACGATGACAGCCTTGGACTTCAATGACTTCAGAGAGTACTTA |
| | | TCTCCAGCATCAGGCTTCCAGAGTTTGCAATTCCGACTATTAGAAAACAAGATAGG |
| | | TGTTCTTCAGAACATGAGAGTCCCTTATAACAGAAGACATTATCGTGATAACTTCA |
| | | AAGGAGAAGAAAATGAACTGCTACTTAAATCTGAGCAGGAAAAGACACTTCTGGAA |
| | | TTAGTGGAGGCATGGCTGGAAAGAACTCCAGGTTTAGAGCCACATGGATTTAACTT |
| | | CTGGGGAAAGCTTGAAAAAAATATCACCAGAGGCCTGGAAGAGGAATTCATAAGGA |
| | | TTCAGGCTAAAGAAGAGTCTGAAGAAAAAGAGGAACAGGTGGCTGAATTTCAGAAG |
| | | CAAAAAGAGGTGCTACTGTCCTTATTTGATGAGAAACGTCATGAACATCTCCTTAG |
| | | TAAAGGTGAAAGACGGCTGTCATACAGAGCACTTCAGGGAGCATTGATGATATATT |
| | | TTTACAGGGAAGAGCCTAGGTTCCAGGTGCCTTTTCAGTTGCTGACTTCTCTTATG |
| | | GACATAGATTCACTGATGACCAAATGGAGATATAACCATGTGTGCATGGTGCACAG |
| | | AATGCTGGGCAGCAAAGCTGGCACCGGTGGTTCCTCAGGCTATCACTACCTGCGAT |
| | | CAACTGTGAGTGATAGGTACAAGGTATTTGTAGATTTATTTAATCTTTCAACATAC |
| | | CTGATTCCCCGACACTGGATACCGAAGATGAACCCAACCATTCACAAATTTCTATA |
| | | TACAGCAGAATACTGTGATAGCTCCTACTTCAGCAGTGATGAATCAGATTAAAATC |
| | | GTCTGCAAAATCTATGAAGAATACTGGTTTCACAGCCTATTTTTTATTTTCTATGG |
| | | ATTTTCATAAATACAGTTTGAATATATGTATGCATATATTGTTCAGCACCACGATG |
| | | CTCTGATTTAATTCTAGAAACAATTTGATTACCTCTTGTTTGTGACAAGACTAAGC |
| | | ATTAAGATGAGAAAGAATACATTTAAATAGTAACATTGTACATAGGGTGTTTTCCT |
| | | ATTAAAAATTCAGTTTCCCCTGAGACTTAATGTAACCACTTAATGTAATCACTATC |
| | | TCATTGTTTCATCTTTATAAACTTGTAAACTTCATCTATTTCAAATATTTTATGCA |
| | | GTACATTATATTATTCTGTACAAAGGCTTTCAAACAAAATTTTTAAAATAATAAAG |
| | | TATTAATCTTTCTCCCTGTA |
| 45 | TK1 | GGCTTACTGCGGGACGGCCTTGGAGAGTACTCGGGTTCGTGAACTTCCCGGAGGCG |
| | | CAATGAGCTGCATTAACCTGCCCACTGTGCTGCCTGGCTCCCCCAGCAAGACCCGG |
| | | GGGCAGATCCAGGTGATTCTCGGGCCGATGTTCTCAGGAAAAAGCACAGAGTTGAT |
| | | GAGACGCGTCCGTCGCTTCCAGATTGCTCAGTACAAGTGCCTGGTGATCAAGTATG |
| | | CCAAAGACACTCGCTACAGCAGCAGCTTCTGCACACATGACCGGAACACCATGGAG |
| | | GCACTGCCCGCCTGCCTGCTCCGAGACGTGGCCCAGGAGGCCCTGGGCGTGGCTGT |
| | | CATAGGCATCGACGAGGGGCAGTTTTTCCCTGACATCGTGGAGTTCTGCGAGGCCA |
| | | TGGCCAACGCCGGGAAGACCGTAATTGTGGCTGCACTGGATGGGACCTTCCAGAGG |
| | | AAGCCATTTGGGGCCATCCTGAACCTGGTGCCGCTGGCCGAGAGCGTGGTGAAGCT |
| | | GACGGCGGTGTGCATGGAGTGCTTCCGGGAAGCCGCCTATACCAAGAGGCTCGGCA |
| | | CAGAGAAGGAGGTCGAGGTGATTGGGGGAGCAGACAAGTACCACTCCGTGTGTCGG |
| | | CTCTGCTACTTCAAGAAGGCCTCAGGCCAGCCTGCCGGGCCGGACAACAAAGAGAA |
| | | CTGCCCAGTGCCAGGAAAGCCAGGGGAAGCCGTGGCTGCCAGGAAGCTCTTTGCCC |
| | | CACAGCAGATTCTGCAATGCAGCCCTGCCAACTGAGGGACCTGCGAGGGCCGCCCG |
| | | CTCCCTTCCTGCCACTGCCGCCTACTGGACGCTGCCCTGCATGCTGCCCAGCCACT |
| | | CCAGGAGGAAGTCGGGAGGCGTGGAGGGTGACCACACCTTGGCCTTCTGGGAACTC |
| | | TCCTTTGTGTGGCTGCCCCACCTGCCGCATGCTCCCTCCTCTCCTACCCACTGGTC |
| | | TGCTTAAAGCTTCCCTCTCAGCTGCTGGGACGATCGCCCAGGCTGGAGCTGGCCCC |
| | | GCTTGGTGGCCTGGGATCTGGCACACTCCCTCTCCTTGGGGTGAGGGACAGAGCCC |
| | | CACGCTGTTGACATCAGCCTGCTTCTTCCCCTCTGCGGCTTTCACTGCTGAGTTTC |
| | | TGTTCTCCCTGGGAAGCCTGTGCCAGCACCTTTGAGCCTTGGCCCACACTGAGGCT |
| | | TAGGCCTCTCTGCCTGGGATGGGCTCCCACCCTCCCCTGAGGATGGCCTGGATTCA |
| | | CGCCCTCTTGTTTCCTTTTGGGCTCAAAGCCCTTCCTACCTCTGGTGATGGTTTCC |
| | | ACAGGAACAACAGCATCTTTCACCAAGATGGGTGGCACCAACCTTGCTGGGACTTG |
| | | GATCCCAGGGGCTTATCTCTTCAAGTGTGGAGAGGGCAGGGTCCACGCCTCTGCTG |
| | | TAGCTTATGAAATTAACTAATTGAAAATTCA |
| 46 | TMEFF2 | AGAGGGATGCGGGCGGCAGAGCTCGAGAGGCGGCTGCCGGGCTGCGGGGCGCCTTG |
| | | ACTCTCCCTCCACCCTGCCTCCTCGGGCTCCACTCGTCTGCCCCTGGACTCCCGTC |
| | | TCCTCCTGTCCTCCGGCTTCCCAGAGCTCCCTCCTTATGGCAGCAGCTTCCCGCGT |
| | | CTCCGGCGCAGCTTCTCAGCGGACGACCCTCTCGCTCCGGGGCTGAGCCCAGTCCC |
| | | TGGATGTTGCTGAAACTCTCGAGATCATGCGCGGGTTTGGCTGCTGCTTCCCCGCC |
| | | GGGTGCCACTGCCACCGCCGCCGCCTCTGCTGCCGCCGTCCGCGGGATGCTCAGTA |
| | | GCCCGCTGCCCGGCCCCCGCGATCCTGTGTTCCTCGGAAGCCGTTTGCTGCTGCAG |
| | | AGTTGCACGAACTAGTCATGGTGCTGTGGGAGTCCCCGCGGCAGTGCAGCAGCTGG |
| | | ACACTTTGCGAGGGCTTTTGCTGGCTGCTGCTGCTGCCCGTCATGCTACTCATCGT |
| | | AGCCCGCCCGGTGAAGCTCGCTGCTTTCCCTACCTCCTTAAGTGACTGCCAAACGC |
| | | CCACCGGCTGGAATTGCTCTGGTTATGATGACAGAGAAAATGATCTCTTCCTCTGT |
| | | GACACCAACACCTGTAAATTTGATGGGGAATGTTTAAGAATTGGAGACACTGTGAC |
| | | TTGCGTCTGTCAGTTCAAGTGCAACAATGACTATGTGCCTGTGTGTGGCTCCAATG |
| | | GGGAGAGCTACCAGAATGAGTGTTACCTGCGACAGGCTGCATGCAAACAGCAGAGT |
| | | GAGATACTTGTGGTGTCAGAAGGATCATGTGCCACAGATGCAGGATCAGGATCTGG |
| | | AGATGGAGTCCATGAAGGCTCTGGAGAAACTAGTCAAAAGGAGACATCCACCTGTG |
| | | ATATTTGCCAGTTTGGTGCAGAATGTGACGAAGATGCCGAGGATGTCTGGTGTGTG |
| | | TGTAATATTGACTGTTCTCAAACCAACTTCAATCCCCTCTGCGCTTCTGATGGGAA |
| | | ATCTTATGATAATGCATGCCAAATCAAAGAAGCATCGTGTCAGAAACAGGAGAAAA |
| | | TTGAAGTCATGTCTTTGGGTCGATGTCAAGATAACACAACTACAACTACTAAGTCT |
| | | GAAGATGGGCATTATGCAAGAACAGATTATGCAGAGAATGCTAACAAATTAGAAGA |
| | | AAGTGCCAGAGAACACCACATACCTTGTCCGGAACATTACAATGGCTTCTGCATGC |
| | | ATGGGAAGTGTGAGCATTCTATCAATATGCAGGAGCCATCTTGCAGGTGTGATGCT |
| | | GGTTATACTGGACAACACTGTGAAAAAAAGGACTACAGTGTTCTATACGTTGTTCC |
| | | CGGTCCTGTACGATTTCAGTATGTCTTAATCGCAGCTGTGATTGGAACAATTCAGA |
| | | TTGCTGTCATCTGTGTGGTGGTCCTCTGCATCACAAGGAAATGCCCCAGAAGCAAC |
| | | AGAATTCACAGACAGAAGCAAAATACAGGGCACTACAGTTCAGACAATACAACAAG |
| | | AGCGTCCACGAGGTTAATCTAAAGGGAGCATGTTTCACAGTGGCTGGACTACCGAG |
| | | AGCTTGGACTACACAATACAGTATTATAGACAAAAGAATAAGACAAGAGATCTACA |
| | | CATGTTGCCTTGCATTTGTGGTAATCTACACCAATGAAAACATGTACTACAGCTAT |
| | | ATTTGATTATGTATGGATATATTTGAAATAGTATACATTGTCTTGATGTTTTTTCT |
| | | GTAATGTAAATAAACTATTTATATCACACAATATAGTTTTTTCTTTCCCATGTATT |
| | | TGTTATATATAATAAATACTCAGTGATGAGAAAAAATTGGCATTCTTAAATTTGCG |
| | | GTATCTCATAACTGTAAATATAATCAGACTAGTACAATCTGTACAGCTACCAATAT |
| | | TTCATGTTTCTTCTCATCTTGAGACAGCACATTAGTTCGTACAGGACTCAGTGGCT |
| | | AGGTTTTGAATGATTCCAAGATCAAGGGAAATGATGGTTATTGGAAAAGAGAAAAA |
| | | ATAATTTACTTTATATCGAGTGAGGATAAAATATTTCCGATCTTTGAATCATCTCT |
| | | ATTTCATCAACTTTCTTCCCTGGTCTTCCATTTTCATCCCTAGAGCAGAAAAATCT |
| | | CTGGCATATAAACTAAATAAAAGAAGAAGGGGAGGGAAAGTGTTTTATAACTCATA |
| | | AAGGAGAGGGAAAGAAAATATTGGTTTTTATTGGGGAAGTAGCTTAGAATCCCCCA |
| | | GTTAAGTGCATATATCTGAACTTACTGAACAAGTTACATACTAGGTATACACAGAG |
| | | TGGCAAAATATATTCCATTTAGGTGGGTGGAATTACCAGGGGAAAAATGTAATAAC |
| | | ACCACTAGATGTGAAACACCAAAATCGTGAATTCTCAAAAGCACCATACAATATGT |
| | | ATAGTATATAGTTCTTTGAAAAGAAGTTAGAATCACAACCAATACCCCATGAATAG |
| | | CTTTGTGGCTAATGCAGCACCATAATTTGTAATGGAACTAAGATGATGATGACGAT |
| | | ATTTCATGAAAACAGAGAGATGTTTTGAGCATATTTATGTGGTGAGGTAAGAAAGA |
| | | AAATTAATCCTATAGCATCTGAAAGACCTCACTGGGAAGTTGGTATGGATTTTTGT |
| | | TTGATTTGTGCATACAAATAGGTATCACAACTTGATCTGGAAAAAATAAGCTGTGA |
| | | AAATTCTCAAGGAATAAGATGAAAATAAATCAATTATTATCATTTAGCTCTGCAAA |
| | | GCTTTCCATGGCTAACACAGTAAATTTAAATAAACTCTCTTTGTCTCCTTCAAA |
| 47 | VSTM2L | GCAGTCGGAGGCGGCCGGCTGGGCGTGCGCTCGCTCCCCGAAGCCGGGGCTGGGCC |
| | | GGAGCCGGGCGAGGGCTGGGAGCTGGGCCGGGTCCGGGGACAGCGGGCGAGGGGCA |
| | | GCTGCCGGAGCCGGGCAGCCAGGCCGCTCAGGGCAGGGGACAGCTGGCGCCGGTTC |
| | | TGCGGTCTCCGGGGCCCAGATGTGAGGCGGCGGCGCCCCCGGCCCGAGAGCGCACG |
| | | ATGGGGGCCCCGCTCGCCGTAGCGCTGGGCGCCCTCCACTACCTGGCACTTTTCCT |
| | | GCAACTCGGCGGCGCCACGCGGCCCGCCGGCCACGCGCCCTGGGACAACCACGTCT |
| | | CCGGCCACGCCCTGTTCACAGAGACACCCCATGACATGACAGCACGGACGGGCGAG |
| | | GACGTGGAGATGGCCTGCTCCTTCCGCGGCAGCGGCTCCCCCTCCTACTCGCTGGA |
| | | GATCCAGTGGTGGTATGTACGGAGCCACCGGGACTGGACCGACAAGCAGGCGTGGG |
| | | CCTCGAACCAGCTAAAAGCATCTCAGCAGGAAGACGCAGGGAAGGAGGCAACCAAA |
| | | ATAAGTGTGGTCAAGGTGGTGGGCAGCAACATCTCCCACAAGCTGCGCCTGTCCCG |
| | | GGTGAAGCCCACGGACGAAGGCACCTACGAGTGCCGCGTCATCGACTTCAGCGACG |
| | | GCAAGGCCCGGCACCACAAGGTCAAGGCCTACCTGCGGGTGCAGCCAGGGGAGAAC |
| | | TCCGTCCTGCATCTGCCCGAAGCCCCTCCCGCCGCGCCCGCCCCGCCGCCCCCCAA |
| | | GCCAGGCAAGGAGCTGAGGAAGCGCTCGGTGGACCAGGAGGCCTGCAGCCTCTAGA |
| | | CTGATGCCCCTGCCCCCGCCCATCCGCCCCCACGCTGTACAGAGTGCATGAGGAGC |
| | | CGCCGGACCACCGGGGACCGACTGCCTGCGTCCAGCCGCGCCCCATCCCCGAGGCC |
| | | GCCTGTGGCCACCATGTCGGCCCTCTTTCCACCACCCCTTGCTCAGCATGTAAGCC |
| | | CCACCCACCCCTGCCCTTTCAGACCCCTGCGGTGACCTGGCTCGGAGAAGGTGGCC |
| | | CTGGGCACCAAGGGGCCAACCGCCCTGAACACTGGGGCAGGGACCATGCTGGGGCC |
| | | CGGGGCCACCCCCTTCCTGTCACCAGCTTCTGTGGAGTCCAGTGTTTTGCTTTGCT |
| | | TGCTTGTCCCCCATCCTGTCCTGAGCCGGGGCCCCCCAGCCTCGCCTCCCTCCTCC |
| | | TACCATCCCTCACTTGGACCTGGGGGTGTGGACAGTGACCCCTCCCTGAATATGGA |
| | | CTTGAATCTTCTGAGCAGAACTAGGGCCTCTCCCCTGGTGAAGACCCAGGGAACCC |
| | | AGGAGGGCCCTTCTGGGGCAGTGGCTCTGCAGGGTCACTCATGGAGGCCTAGGGGA |
| | | ACAGCGAGATGCCCCACCACCTCCTGGCGAGTCCTTCCTGTTCAGCTCCCTGTGCG |
| | | ACCCTCCAGGGATGCAGGGGATCCAGGATTCTCTGCCCTGTCACACGGCGAGTCAG |
| | | AAGGGAGGGGCCTTTCCCTCGGACCCATGGCCCCAGGCAGAGTTTTGCACCAGCAG |
| | | GACCCCTTTGAGGGCCTTCAAGGCTCTCCCAGGAGTCCCCCTCTGCCGGCCCCCCA |
| | | ATGCCCCAGCTCCCTCTTGGGTCCTGTGCCAAGTCCGCCCCAGGGCCTGGGGCTGT |
| | | TGGGAGCCAAGGGCCCCCTGGTACTCAGTTCCCTCACGATTCCCGATCACGGGCAC |
| | | ACCTGCCCCCTGGTTATTTGTAAATATTTCTATTGGACCCAATTCTCCTCGGAATT |
| | | GGCTGGCACCTCTGGCTGCCGCAGCTCAGTGATGACGTGGGGGAGGTGGGAGAGGC |
| | | CGAGGGCTTTGCCTAGGGGTGGGTTGCCCTGTATACATGATCCAGTCTGTGACTAC |
| | | CAGCCAACCTGAATAAAGCGGTTTTAAAAAAA |
| 48 | KLK2 | AAACTCACCACCTGGCCGTGGACACCTGTGTCAGCATGTGGGACCTGGTTCTCTCC |
| | | ATCGCCTTGTCTGTGGGGTGCACTGGTGCCGTGCCCCTCATCCAGTCTCGGATTGT |
| | | GGGAGGCTGGGAGTGTGAGAAGCATTCCCAACCCTGGCAGGTGGCTGTGTACAGTC |
| | | ATGGATGGGCACACTGTGGGGGTGTCCTGGTGCACCCCCAGTGGGTGCTCACAGCT |
| | | GCCCATTGCCTAAAGAAGAATAGCCAGGTCTGGCTGGGTCGGCACAACCTGTTTGA |
| | | GCCTGAAGACACAGGCCAGAGGGTCCCTGTCAGCCACAGCTTCCCACACCCGCTCT |
| | | ACAATATGAGCCTTCTGAAGCATCAAAGCCTTAGACCAGATGAAGACTCCAGCCAT |
| | | GACCTCATGCTGCTCCGCCTGTCAGAGCCTGCCAAGATCACAGATGTTGTGAAGGT |
| | | CCTGGGCCTGCCCACCCAGGAGCCAGCACTGGGGACCACCTGCTACGCCTCAGGCT |
| | | GGGGCAGCATCGAACCAGAGGAGTTCTTGCGCCCCAGGAGTCTTCAGTGTGTGAGC |
| | | CTCCATCTCCTGTCCAATGACATGTGTGCTAGAGCTTACTCTGAGAAGGTGACAGA |
| | | GTTCATGTTGTGTGCTGGGCTCTGGACAGGTGGTAAAGACACTTGTGGGGGTGATT |
| | | CTGGGGGTCCACTTGTCTGTAATGGTGTGCTTCAAGGTATCACATCATGGGGCCCT |
| | | GAGCCATGTGCCCTGCCTGAAAAGCCTGCTGTGTACACCAAGGTGGTGCATTACCG |
| | | GAAGTGGATCAAGGACACCATCGCAGCCAACCCCTGAGTGCCCCTGTCCCACCCCT |
| | | ACCTCTAGTAAATTTAAGTCCACCTCACGTTCTGGCATCACTTGGCCTTTCTGGAT |
| | | GCTGGACACCTGAAGCTTGGAACTCACCTGGCCGAAGCTCGAGCCTCCTGAGTCCT |
| | | ACTGACCTGTGCTTTCTGGTGTGGAGTCCAGGGCTGCTAGGAAAAGGAATGGGCAG |
| | | ACACAGGTGTATGCCAATGTTTCTGAAATGGGTATAATTTCGTCCTCTCCTTCGGA |
| | | ACACTGGCTGTCTCTGAAGACTTCTCGCTCAGTTTCAGTGAGGACACACACAAAGA |
| | | CGTGGGTGACCATGTTGTTTGTGGGGTGCAGAGATGGGAGGGGTGGGGCCCACCCT |
| | | GGAAGAGTGGACAGTGACACAAGGTGGACACTCTCTACAGATCACTGAGGATAAGC |
| | | TGGAGCCACAATGCATGAGGCACACACACAGCAAGGATGACGCTGTAAACATAGCC |
| | | CACGCTGTCCTGGGGGCACTGGGAAGCCTAGATAAGGCCGTGAGCAGAAAGAAGGG |
| | | GAGGATCCTCCTATGTTGTTGAAGGAGGGACTAGGGGGAGAAACTGAAAGCTGATT |
| | | AATTACAGGAGGTTTGTTCAGGTCCCCCAAACCACCGTCAGATTTGATGATTTCCT |
| | | AGCAGGACTTACAGAAATAAAGAGCTATCATGCTGTGGTTTATTATGGTTTGTTAC |
| | | ATTGATAGGATACATACTGAAATCAGCAAACAAAACAGATGTATAGATTAGAGTGT |
| | | GGAGAAAACAGAGGAAAACTTGCAGTTACGAAGACTGGCAACTTGGCTTTACTAAG |
| | | TTTTCAGACTGGCAGGAAGTCAAACCTATTAGGCTGAGGACCTTGTGGAGTGTAGC |
| | | TGATCCAGCTGATAGAGGAACTAGCCAGGTGGGGGCCTTTCCCTTTGGATGGGGGG |
| | | CATATCTGACAGTTATTCTCTCCAAGTGGAGACTTACGGACAGCATATAATTCTCC |
| | | CTGCAAGGATGTATGATAATATGTACAAAGTAATTCCAACTGAGGAAGCTCACCTG |
| | | ATCCTTAGTGTCCAGGGTTTTTACTGGGGGTCTGTAGGACGAGTATGGAGTACTTG |
| | | AATAATTGACCTGAAGTCCTCAGACCTGAGGTTCCCTAGAGTTCAAACAGATACAG |
| | | CATGGTCCAGAGTCCCAGATGTACAAAAACAGGGATTCATCACAAATCCCATCTTT |
| | | AGCATGAAGGGTCTGGCATGGCCCAAGGCCCCAAGTATATCAAGGCACTTGGGCAG |
| | | AACATGCCAAGGAATCAAATGTCATCTCCCAGGAGTTATTCAAGGGTGAGCCCTTT |
| | | ACTTGGGATGTACAGGCTTTGAGCAGTGCAGGGCTGCTGAGTCAACCTTTTATTGT |
| | | ACAGGGGATGAGGGAAAGGGAGAGGATGAGGAAGCCCCCCTGGGGATTTGGTTTGG |
| | | TCTTGTGATCAGGTGGTCTATGGGGCTATCCCTACAAAGAAGAATCCAGAAATAGG |
| | | GGCACATTGAGGAATGATACTGAGCCCAAAGAGCATTCAATCATTGTTTTATTTGC |
| | | CTTCTTTTCACACCATTGGTGAGGGAGGGATTACCACCCTGGGGTTATGAAGATGG |
| | | TTGAACACCCCACACATAGCACCGGAGATATGAGATCAACAGTTTCTTAGCCATAG |
| | | AGATTCACAGCCCAGAGCAGGAGGACGCTGCACACCATGCAGGATGACATGGGGGA |
| | | TGCGCTCGGGATTGGTGTGAAGAAGCAAGGACTGTTAGAGGCAGGCTTTATAGTAA |
| | | CAAGACGGTGGGGCAAACTCTGATTTCCGTGGGGGAATGTCATGGTCTTGCTTTAC |
| | | TAAGTTTTGAGACTGGCAGGTAGTGAAACTCATTAGGCTGAGAACCTTGTGGAATG |
| | | CAGCTGACCCAGCTGATAGAGGAAGTAGCCAGGTGGGAGCCTTTCCCAGTGGGTGT |
| | | GGGACATATCTGGCAAGATTTTGTGGCACTCCTGGTTACAGATACTGGGGCAGCAA |
| | | ATAAAACTGAATCTTGTTTTCAGACCTTA |
| 49 | NUDT8 | AGTGTCCCGGCCGCGCAGGACTTGACATGCTGCCCGACTGCCTGTCGGCCGAGGGC |
| | | GAGCTGCGCTGCCGCCGGCTGCTGGCAGGGGCCACGGCCCGGCTCCGCGCGCGGCC |
| | | CGCGTCGGCCGCGGTGCTCGTGCCGCTCTGCTCAGTGCGTGGGGTCCCGGCGCTGC |
| | | TGTACACGCTGCGGTCCAGCCGCCTGACCGGGAGGCACAAGGGCGACGTCAGTTTC |
| | | CCAGGCGGCAAGTGCGACCCGGCTGACCAAGATGTGGTGCACACGGCCCTGCGGGA |
| | | AACCCGGGAGGAGCTGGGCCTGGCAGTGCCCGAGGAGCACGTGTGGGGCCTGCTGC |
| | | GGCCTGTGTATGATCCGCAAAAGGCCACCGTGGTGCCAGTGCTTGCTGGTGTAGGC |
| | | CCACTGGATCCCCAGAGCCTCAGGCCCAACTCGGAGGAGGTAGATGAGGTGTTTGC |
| | | ACTGCCGCTGGCCCACCTGCTGCAGACGCAGAATCAGGGCTATACCCACTTCTGCC |
| | | GGGGTGGCCACTTCCGCTACACACTACCCGTCTTCCTGCATGGACCACACCGGGTC |
| | | TGGGGCCTCACAGCTGTCATCACTGAGTTTGCCCTGCAGCTGCTGGCACCTGGTAC |
| | | CTACCAGCCCCGCCTGGCCGGCCTGACCTGCTCAGGGGCTGAGGGTCTGGCCCGCC |
| | | CTAAGCAGCCCCTGGCTTCACCCTGTCAGGCCAGCTCCACTCCAGGACTGAATAAA |
| | | GGTCTTTGACAGCTCTA |
| 50 | EEF1A2 | CCCTCTGGCTGAGACCTCGGCTCCGGAATCACTGCAGCCCCCCTCGCCCTGAGCCA |
| | | GAGCACCCCGGGTCCCGCCAGCCCCTCACACTCCCAGCAAAATGGGCAAGGAGAAG |
| | | ACCCACATCAACATCGTGGTCATCGGCCACGTGGACTCCGGAAAGTCCACCACCAC |
| | | GGGCCACCTCATCTACAAATGCGGAGGTATTGACAAAAGGACCATTGAGAAGTTCG |
| | | AGAAGGAGGCGGCTGAGATGGGGAAGGGATCCTTCAAGTATGCCTGGGTGCTGGAC |
| | | AAGCTGAAGGCGGAGCGTGAGCGCGGCATCACCATCGACATCTCCCTCTGGAAGTT |
| | | CGAGACCACCAAGTACTACATCACCATCATCGATGCCCCCGGCCACCGCGACTTCA |
| | | TCAAGAACATGATCACGGGTACATCCCAGGCGGACTGCGCAGTGCTGATCGTGGCG |
| | | GCGGGCGTGGGCGAGTTCGAGGCGGGCATCTCCAAGAATGGGCAGACGCGGGAGCA |
| | | TGCCCTGCTGGCCTACACGCTGGGTGTGAAGCAGCTCATCGTGGGCGTGAACAAAA |
| | | TGGACTCCACAGAGCCGGCCTACAGCGAGAAGCGCTACGACGAGATCGTCAAGGAA |
| | | GTCAGCGCCTACATCAAGAAGATCGGCTACAACCCGGCCACCGTGCCCTTTGTGCC |
| | | CATCTCCGGCTGGCACGGTGACAACATGCTGGAGCCCTCCCCCAACATGCCGTGGT |
| | | TCAAGGGCTGGAAGGTGGAGCGTAAGGAGGGCAACGCAAGCGGCGTGTCCCTGCTG |
| | | GAGGCCCTGGACACCATCCTGCCCCCCACGCGCCCCACGGACAAGCCCCTGCGCCT |
| | | GCCGCTGCAGGACGTGTACAAGATTGGCGGCATTGGCACGGTGCCCGTGGGCCGGG |
| | | TGGAGACCGGCATCCTGCGGCCGGGCATGGTGGTGACCTTTGCGCCAGTGAACATC |
| | | ACCACTGAGGTGAAGTCAGTGGAGATGCACCACGAGGCTCTGAGCGAAGCTCTGCC |
| | | CGGCGACAACGTCGGCTTCAATGTGAAGAACGTGTCGGTGAAGGACATCCGGCGGG |
| | | GCAACGTGTGTGGGGACAGCAAGTCTGACCCGCCGCAGGAGGCTGCTCAGTTCACC |
| | | TCCCAGGTCATCATCCTGAACCACCCGGGGCAGATTAGCGCCGGCTACTCCCCGGT |
| | | CATCGACTGCCACACAGCCCACATCGCCTGCAAGTTTGCGGAGCTGAAGGAGAAGA |
| | | TTGACCGGCGCTCTGGCAAGAAGCTGGAGGACAACCCCAAGTCCCTGAAGTCTGGA |
| | | GACGCGGCCATCGTGGAGATGGTGCCGGGAAAGCCCATGTGTGTGGAGAGCTTCTC |
| | | CCAGTACCCGCCTCTCGGCCGCTTCGCCGTGCGCGACATGAGGCAGACGGTGGCCG |
| | | TAGGCGTCATCAAGAACGTGGAGAAGAAGAGCGGCGGCGCCGGCAAGGTCACCAAG |
| | | TCGGCGCAGAAGGCGCAGAAGGCGGGCAAGTGAAGCGCGGGCGCCCGCGGCGCGAC |
| | | CCTCCCCGGCGGTGCCGCGCTCCGAACCCCGGGCCCGGGCCCCCGCCCCGCCCCCG |
| | | CCCCGCGCGCCGGTCCGGCGCCCCGCACCCCCGCCAGGCGCATGTCTGCACCTCCG |
| | | CTTGCCAGAGGCCCTCGGTCAGCGACTGGATGCTCGCCATCAAGGTCCAGTGGAAG |
| | | TTCTTCAAGAGGAAAGGCGCCCCCGCCCCAGGCTTCCGCGCCCAGCGCTCGCCACG |
| | | CTCAGTGCCCGTTTTACCAATAAACTGAGCGACCCCA |
| 51 | SPDEF | CTTCATCTCGCGGCTGTCTGACTTCCTCCCAGCACATTCCTGCACTCTGCCGTGTC |
| | | CACACTGCCCCACAGACCCAGTCCTCCAAGCCTGCTGCCAGCTCCCTGCAAGCCCC |
| | | TCAGGTTGGGCCTTGCCACGGTGCCAGCAGGCAGCCCTGGGCTGGGGGTAGGGGAC |
| | | TCCCTACAGGCACGCAGCCCTGAGACCTCAGAGGGCCACCCCTTGAGGGTGGCCAG |
| | | GCCCCCAGTGGCCAACCTGAGTGCTGCCTCTGCCACCAGCCCTGCTGGCCCCTGGT |
| | | TCCGCTGGCCCCCCAGATGCCTGGCTGAGACACGCCAGTGGCCTCAGCTGCCCACA |
| | | CCTCTTCCCGGCCCCTGAAGTTGGCACTGCAGCAGACAGCTCCCTGGGCACCAGGC |
| | | AGCTAACAGACACAGCCGCCAGCCCAAACAGCAGCGGCATGGGCAGCGCCAGCCCG |
| | | GGTCTGAGCAGCGTATCCCCCAGCCACCTCCTGCTGCCCCCCGACACGGTGTCGCG |
| | | GACAGGCTTGGAGAAGGCGGCAGCGGGGGCAGTGGGTCTCGAGAGACGGGACTGGA |
| | | GTCCCAGTCCACCCGCCACGCCCGAGCAGGGCCTGTCCGCCTTCTACCTCTCCTAC |
| | | TTTGACATGCTGTACCCTGAGGACAGCAGCTGGGCAGCCAAGGCCCCTGGGGCCAG |
| | | CAGTCGGGAGGAGCCACCTGAGGAGCCTGAGCAGTGCCCGGTCATTGACAGCCAAG |
| | | CCCCAGCGGGCAGCCTGGACTTGGTGCCCGGCGGGCTGACCTTGGAGGAGCACTCG |
| | | CTGGAGCAGGTGCAGTCCATGGTGGTGGGCGAAGTGCTCAAGGACATCGAGACGGC |
| | | CTGCAAGCTGCTCAACATCACCGCAGATCCCATGGACTGGAGCCCCAGCAATGTGC |
| | | AGAAGTGGCTCCTGTGGACAGAGCACCAATACCGGCTGCCCCCCATGGGCAAGGCC |
| | | TTCCAGGAGCTGGCGGGCAAGGAGCTGTGCGCCATGTCGGAGGAGCAGTTCCGCCA |
| | | GCGCTCGCCCCTGGGTGGGGATGTGCTGCACGCCCACCTGGACATCTGGAAGTCAG |
| | | CGGCCTGGATGAAAGAGCGGACTTCACCTGGGGCGATTCACTACTGTGCCTCGACC |
| | | AGTGAGGAGAGCTGGACCGACAGCGAGGTGGACTCATCATGCTCCGGGCAGCCCAT |
| | | CCACCTGTGGCAGTTCCTCAAGGAGTTGCTACTCAAGCCCCACAGCTATGGCCGCT |
| | | TCATTAGGTGGCTCAACAAGGAGAAGGGCATCTTCAAAATTGAGGACTCAGCCCAG |
| | | GTGGCCCGGCTGTGGGGCATCCGCAAGAACCGTCCCGCCATGAACTACGACAAGCT |
| | | GAGCCGCTCCATCCGCCAGTATTACAAGAAGGGCATCATCCGGAAGCCAGACATCT |
| | | CCCAGCGCCTCGTCTACCAGTTCGTGCACCCCATCTGAGTGCCTGGCCCAGGGCCT |
| | | GAAACCCGCCCTCAGGGGCCTCTCTCCTGCCTGCCCTGCCTCAGCCAGGCCCTGAG |
| | | ATGGGGGAAAACGGGCAGTCTGCTCTGCTGCTCTGACCTTCCAGAGCCCAAGGTCA |
| | | GGGAGGGGCAACCAACTGCCCCAGGGGGATATGGGTCCTCTGGGGCCTTCGGGACC |
| | | CTGGGGCAGGGGTGCTTCCTCCTCAGGCCCAGCTGCTCCCCTGGAGGACAGAGGGA |
| | | GACAGGGCTGCTCCCCAACACCTGCCTCTGACCCCAGCATTTCCAGAGCAGAGCCT |
| | | ACAGAAGGGCAGTGACTCGACAAAGGCCACAGGCAGTCCAGGCCTCTCTCTGCTCC |
| | | ATCCCCCTGCCTCCCATTCTGCACCACACCTGGCATGGTGCAGGGAGACATCTGCA |
| | | CCCCTGAGTTGGGCAGCCAGGAGTGCCCCCGGGAATGGATAATAAAGATACTAGAG |
| | | AACTGA |
| 52 | GAPDH | GCTCTCTGCTCCTCCTGTTCGACAGTCAGCCGCATCTTCTTTTGCGTCGCCAGCCG |
| | | AGCCACATCGCTCAGACACCATGGGGAAGGTGAAGGTCGGAGTCAACGGATTTGGT |
| | | CGTATTGGGCGCCTGGTCACCAGGGCTGCTTTTAACTCTGGTAAAGTGGATATTGT |
| | | TGCCATCAATGACCCCTTCATTGACCTCAACTACATGGTTTACATGTTCCAATATG |
| | | ATTCCACCCATGGCAAATTCCATGGCACCGTCAAGGCTGAGAACGGGAAGCTTGTC |
| | | ATCAATGGAAATCCCATCACCATCTTCCAGGAGCGAGATCCCTCCAAAATCAAGTG |
| | | GGGCGATGCTGGCGCTGAGTACGTCGTGGAGTCCACTGGCGTCTTCACCACCATGG |
| | | AGAAGGCTGGGGCTCATTTGCAGGGGGGAGCCAAAAGGGTCATCATCTCTGCCCCC |
| | | TCTGCTGATGCCCCCATGTTCGTCATGGGTGTGAACCATGAGAAGTATGACAACAG |
| | | CCTCAAGATCATCAGCAATGCCTCCTGCACCACCAACTGCTTAGCACCCCTGGCCA |
| | | AGGTCATCCATGACAACTTTGGTATCGTGGAAGGACTCATGACCACAGTCCATGCC |
| | | ATCACTGCCACCCAGAAGACTGTGGATGGCCCCTCCGGGAAACTGTGGCGTGATGG |
| | | CCGCGGGGCTCTCCAGAACATCATCCCTGCCTCTACTGGCGCTGCCAAGGCTGTGG |
| | | GCAAGGTCATCCCTGAGCTGAACGGGAAGCTCACTGGCATGGCCTTCCGTGTCCCC |
| | | ACTGCCAACGTGTCAGTGGTGGACCTGACCTGCCGTCTAGAAAAACCTGCCAAATA |
| | | TGATGACATCAAGAAGGTGGTGAAGCAGGCGTCGGAGGGCCCCCTCAAGGGCATCC |
| | | TGGGCTACACTGAGCACCAGGTGGTCTCCTCTGACTTCAACAGCGACACCCACTCC |
| | | TCCACCTTTGACGCTGGGGCTGGCATTGCCCTCAACGACCACTTTGTCAAGCTCAT |
| | | TTCCTGGTATGACAACGAATTTGGCTACAGCAACAGGGTGGTGGACCTCATGGCCC |
| | | ACATGGCCTCCAAGGAGTAAGACCCCTGGACCACCAGCCCCAGCAAGAGCACAAGA |
| | | GGAAGAGAGAGACCCTCACTGCTGGGGAGTCCCTGCCACACTCAGTCCCCCACCAC |
| | | ACTGAATCTCCCCTCCTCACAGTTGCCATGTAGACCCCTTGAAGAGGGGAGGGGCC |
| | | TAGGGAGCCGCACCTTGTCATGTACCATCAATAAAGTACCCTGTGCTCAACCA |
| 53 | LBH | GCTGAGTGCTCAGTGGAGAGCGGGGAGTTGTGTCCACCTTGCCGACGTCGCTAGCC |
| | | GTGGGGCTGTCCTGGGAAGGCGGACGGCGAGCGCCCGGTGTCCGCACTCGGCCGCC |
| | | TGCCGTGCCCGTCTGCGCCCGTGTCATCCTCACTCGGGACGCAGGGACCGTTTTTA |
| | | AATCACAGGGGCGTGTGTCAGCCTGCCCTAGGACTTCATGTCTATATATTTCCCCA |
| | | TTCACTGCCCCGACTATCTGAGATCGGCCAAGATGACTGAGGTGATGATGAACACC |
| | | CAGCCCATGGAGGAGATCGGCCTCAGCCCCCGCAAGGATGGCCTTTCCTACCAGAT |
| | | CTTCCCAGACCCGTCAGATTTTGACCGCTGCTGCAAACTGAAGGACCGTCTGCCCT |
| | | CCATAGTGGTGGAACCCACAGAAGGGGAGGTGGAGAGCGGGGAGCTCCGGTGGCCC |
| | | CCTGAGGAGTTCCTGGTCCAGGAGGATGAGCAAGATAACTGCGAAGAGACAGCGAA |
| | | AGAAAATAAAGAGCAGTAGAGTCCCTGTGGACTCCCATGGGTCATACCAGCCAGCA |
| | | TCTGTTCCTGAACTGTGTTTTTCCCATCATGACGGAAGAAGAGAGTGAGCCGCAAT |
| | | TGTTCTGAAAATGTCAAACGAGGCTTCTGTTTTGCACCTGCAGATCACCGAGTTGG |
| | | TTTTCTTTTCTTTTCTTGCCTTTTTTTTTTTTTGAAATTTGCCGAGCAGTGGAGCC |
| | | CTCTGACAATTTGCAAGGCCCTCTGAGAAAGGAAGCTGCTTAGAGCCAGGGGGTTA |
| | | GTGGGTGAGGGGAGCGAGTGCTGTTTTTGAGATCATTATCTGAACTCAGGCAGCCT |
| | | AGTAGAGGCAGTGGTGGGATTCCAATGGGTCTTGGTGGGTGGGAGGTGGGGCATGT |
| | | GCAAAGCAAGCAAGGAACATTTGGGGTAAGAAAACAAACATGAGGCAAAAGAAAAA |
| | | ATACATGTTTTTAAGAAAACATTGAGCAGAGAACTGCAGCCAGGATGCGCTCAGCA |
| | | GACATTCACTCTGGCTGCTGGGACATCAGAAAACAAAGTCTTCATCTCTCTCTCCA |
| | | GTTTCACCCACCCCACCCTTTGCTTTCATTTCAGGTGTGTTGGTCTATATGACAGG |
| | | GAGGAGAGTAAAGGAGAGCAGGAGCAATTGGCTGCCTGCAAAGCCAGCTGGAGGTG |
| | | AAGTGCAGGAAAGGAAAGGTCACCCCATTCTACTCCATGGCCTCTCTGCTCCCAGC |
| | | TGTGGTAGGCTCACATAGCCAGTGTGATCGGTTTTTAAGAGGCAGTGCTTTTCAGC |
| | | TTTTCTCCCTGATATATCCATTTTGCTTCCCAGCACTTTTTAGGAGTAGTGAGAGC |
| | | ACTTCCTGCCCTTGTTGGAAGCCCCAGGGTGGACACTCAGCACGAAGGTCTCTCCC |
| | | TTAACTGCTGCCCTTCCAAGACTTGCTCCCGAGATGGAGTGGGCGTGGTCTTCCAG |
| | | GCTGGCCCTTCCTTCTCCTCACCGCCACCTTCCCTGCCCCAGCCCCAGCAGCCATG |
| | | GGTACATGGGTCCCCAGCTCACCTATGGATTCCCGCCAGTCTGCCCAGCTGCAGTA |
| | | CTCACGCCCCATGGGGGATCTTGGTCTGTTTTTCTTGTGGGAGCCTAGTGGAGAGC |
| | | AGACGTGGCTTTTTATGTGTCTTGTTGGGGAGGTGACTTGCATGGTGGGGACAAGG |
| | | CTGTCGTGGCAACCTTGGGATCGAGTTTGAGACTAAAGGATGTCATGAGATCCCTG |
| | | GCTTCTCCCCATGTTGTTCCCGGACAAGGGCAGAAGGGAGGCATGGCAAGGGACCT |
| | | CTGCTGTCCTTACTCAACAGTGGTCCTCATCCCTCCCCACCTCCCACTGCTTCCTG |
| | | CAAGGGCACCAGTTGTATGAGAAAGTTGGCCTTTGGACTTAGGATTTCTTATTGTA |
| | | GCTAAGAGCCATCTGAAGCAGCAGGTTGCAGGACAAATGCTTCAGTCCGCCGAGAG |
| | | CAGTACCGTGTGGCCAAGAGGTGGACTCAGAGCCTTCCTTGAGCTAAACTCGGCCA |
| | | ACCAAGGCACGCAGCATGTCCCCTCAGGTCTCCAGTCAGTCCAGGTTGACCCTCAG |
| | | TTCTGGACGTGTGTATATAGCTGTATTTAATACCTCAAGGTCATTGTGGCTCTGGG |
| | | GATGCCGGGGCAGGAGGACGAGGGTGCGCTGTGGACACAGCAGTCCGCGGAATTCC |
| | | GTTCTGGGAAGCCAATGGTCGCCGGCACCCCTTGCTTCCTCCCTCTGTTGTCTGCC |
| | | TGTGTGACACACATCAATGGCAATAACTTCTTCCAACTCCTCGCAGAAGTGGGAGA |
| | | GGCCGGCAGCCTGCACCGAGAGGGGCTTTCCTCTCTCTTGCTCCCCGCTTCGTTCT |
| | | GTTTTGGCTGCAGAGAGTGGTTCATCCATACTCTCATTCCCTCGCCTCCCCTTGTG |
| | | GACGGGGGTCTTGCCTTTTCAATTCCTGTGTTTTGGTGTCTTCCCTTATCTGCTAC |
| | | CCTGAATCACCTGTCCTGGTCTTGCTGTGTGATGGGAACATGCTTGTAAACTGCGT |
| | | AACAAATCTACTTTGTGTATGTGTCTGTTTATGGGGGTGGTTTATTATTTTTGCTG |
| | | GTCCCTAGACCACTTTGTATGACCGTTTGCAGTCTGAGCAGGCCAGGGGCTGACAG |
| | | CTAATGTCAGGACCCTCAGCGGTGGAGCCTGCTGGGGGGACCCAGCTGCTCTTGGA |
| | | CAAGTGGCTGAGCTCCTATCTGGCCTCCTCTTTTTTTTTTTTTCAAGTAATTTGTG |
| | | TGTATTTCTAACTGATTGTATTGAAAAAATTCCTAGTATTTCAGTAAAAATGCCTG |
| | | TTGTGAGATGAACCTCCTGTAACTTCTATCTGTTCTTTTTTGAGGCTCAGGGAGAA |
| | | ACTAGCATTTTTTTTTTTCCAAACTACTTTTTGTCACTGTGACAGTTGTAAATAAA |
| | | GTTTGAAAATGCTTTCCA |
| 54 | HPN | GGTGAGGCAGCCTGGCCTAGCAGGCCCCACGCCACCGCCTCTGCCTCCAGGCCGCC |
| | | CGCTGCTGCGGGGCCACCATGCTCCTGCCCAGGCCTGGAGACTGACCCGACCCCGG |
| | | CACTACCTCGAGGCTCCGCCCCCACCTGCTGGACCCCAGGGTCCCACCCTGGCCCA |
| | | GGAGGTCAGCCAGGGAATCATTAACAAGAGGCAGTGACATGGCGCAGAAGGAGGGT |
| | | GGCCGGACTGTGCCATGCTGCTCCAGACCCAAGGTGGCAGCTCTCACTGCGGGGAC |
| | | CCTGCTACTTCTGACAGCCATCGGGGCGGCATCCTGGGCCATTGTGGCTGTTCTCC |
| | | TCAGGAGTGACCAGGAGCCGCTGTACCCAGTGCAGGTCAGCTCTGCGGACGCTCGG |
| | | CTCATGGTCTTTGACAAGACGGAAGGGACGTGGCGGCTGCTGTGCTCCTCGCGCTC |
| | | CAACGCCAGGGTAGCCGGACTCAGCTGCGAGGAGATGGGCTTCCTCAGGGCACTGA |
| | | CCCACTCCGAGCTGGACGTGCGAACGGCGGGCGCCAATGGCACGTCGGGCTTCTTC |
| | | TGTGTGGACGAGGGGAGGCTGCCCCACACCCAGAGGCTGCTGGAGGTCATCTCCGT |
| | | GTGTGATTGCCCCAGAGGCCGTTTCTTGGCCGCCATCTGCCAAGACTGTGGCCGCA |
| | | GGAAGCTGCCCGTGGACCGCATCGTGGGAGGCCGGGACACCAGCTTGGGCCGGTGG |
| | | CCGTGGCAAGTCAGCCTTCGCTATGATGGAGCACACCTCTGTGGGGGATCCCTGCT |
| | | CTCCGGGGACTGGGTGCTGACAGCCGCCCACTGCTTCCCGGAGCGGAACCGGGTCC |
| | | TGTCCCGATGGCGAGTGTTTGCCGGTGCCGTGGCCCAGGCCTCTCCCCACGGTCTG |
| | | CAGCTGGGGGTGCAGGCTGTGGTCTACCACGGGGGCTATCTTCCCTTTCGGGACCC |
| | | CAACAGCGAGGAGAACAGCAACGATATTGCCCTGGTCCACCTCTCCAGTCCCCTGC |
| | | CCCTCACAGAATACATCCAGCCTGTGTGCCTCCCAGCTGCCGGCCAGGCCCTGGTG |
| | | GATGGCAAGATCTGTACCGTGACGGGCTGGGGCAACACGCAGTACTATGGCCAACA |
| | | GGCCGGGGTACTCCAGGAGGCTCGAGTCCCCATAATCAGCAATGATGTCTGCAATG |
| | | GCGCTGACTTCTATGGAAACCAGATCAAGCCCAAGATGTTCTGTGCTGGCTACCCC |
| | | GAGGGTGGCATTGATGCCTGCCAGGGCGACAGCGGTGGTCCCTTTGTGTGTGAGGA |
| | | CAGCATCTCTCGGACGCCACGTTGGCGGCTGTGTGGCATTGTGAGTTGGGGCACTG |
| | | GCTGTGCCCTGGCCCAGAAGCCAGGCGTCTACACCAAAGTCAGTGACTTCCGGGAG |
| | | TGGATCTTCCAGGCCATAAAGACTCACTCCGAAGCCAGCGGCATGGTGACCCAGCT |
| | | CTGACCGGTGGCTTCTCGCTGCGCAGCCTCCAGGGCCCGAGGTGATCCCGGTGGTG |
| | | GGATCCACGCTGGGCCTAGGATGGGACGTTTTTCTTCTTGGGCCCGGTCCACAGGT |
| | | CCAAGGACACCCTCCCTCCAGGGTCCTCTCTTCCACAGTGGCGGGCCCACTCAGCC |
| | | CCGAGACCACCCAACCTCACCCTCCTGACCCCCATGTAAATATTGTTCTGCTGTCT |
| | | GGGACTCCTGTCTAGGTGCCCCTGATGACGGGATGCTCTTTAAATAATAAAGATGG |
| | | TTTTGATTAA |

In some embodiments, the methods described herein comprise detecting an amount of expression of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 of genes ACSM1, AMACR, AR, COL9A2, CRISP3, CST2, DLX1, ETV1, F5, GDF15, GLYATL1, GOLM1, GRIN3A, LINC00993, LRRN1, MIPEP, MS4A8, MYO6, PCA3.1, PDLIM5, PEX10, PLA1A, PLA2G7, PRCAT47, SPINK1, TDO2, TK1, TMEFF2, TRGV9.1, VSTM2L, TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, HOXC6, and KLK3.

In some embodiments, the methods described herein comprise detecting an amount of expression of each of genes ACSM1, AMACR, AR, COL9A2, CRISP3, CST2, DLX1, ETV1, F5, GDF15, GLYATL1, GOLM1, GRIN3A, LINC00993, LRRN1, MIPEP, MS4A8, MYO6, PCA3.1, PDLIM5, PEX10, PLA1A, PLA2G7, PRCAT47, SPINK1, TDO2, TK1, TMEFF2, TRGV9.1, VSTM2L, TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, HOXC6, and KLK3.

In some embodiments, detecting an amount of expression of one or more of genes ACSM1, AMACR, AR, COL9A2, CRISP3, CST2, DLX1, ETV1, F5, GDF15, GLYATL1, GOLM1, GRIN3A, LINC00993, LRRN1, MIPEP, MS4A8, MYO6, PCA3.1, PDLIM5, PEX10, PLA1A, PLA2G7, PRCAT47, SPINK1, TDO2, TK1, TMEFF2, TRGV9.1, VSTM2L, TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, HOXC6, and KLK3 comprises detecting an RNA expressed by the one or more of the genes or an amount of the RNA's expression. In some embodiments, the RNA is mRNA. In some embodiments, the RNA is miRNA. In some embodiments, the RNA is siRNA. In some embodiments, the RNA is circular RNA. In some embodiments, the RNA is lncRNA. In some embodiments, the RNA is piRNA. In some embodiments, the RNA is rRNA. In some embodiments, the RNA is tRNA. In some embodiments, the RNA is hnRNA. In some embodiments, the RNA is ncRNA.

In some embodiments, detecting an amount of expression of one or more of genes ACSM1, AMACR, AR, COL9A2, CRISP3, CST2, DLX1, ETV1, F5, GDF15, GLYATL1, GOLM1, GRIN3A, LINC00993, LRRN1, MIPEP, MS4A8, MYO6, PCA3.1, PDLIM5, PEX10, PLA1A, PLA2G7, PRCAT47, SPINK1, TDO2, TK1, TMEFF2, TRGV9.1, VSTM2L, TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, HOXC6, and KLK3 comprises detecting a cDNA reverse transcribed from an RNA expressed by the one or more of the genes or an amount of the cDNA. In some embodiments, the RNA is mRNA. In some embodiments, the RNA is miRNA. In some embodiments, the RNA is siRNA. In some embodiments, the RNA is circular RNA. In some embodiments, the RNA is lncRNA. In some embodiments, the RNA is piRNA. In some embodiments, the RNA is rRNA. In some embodiments, the RNA is tRNA. In some embodiments, the RNA is hnRNA. In some embodiments, the RNA is ncRNA.

In some embodiments, the methods described herein comprise detecting an amount of expression of 1, 2, 3, 4, 5, 6, 7, 8, 9, 10, 11, 12, 13, 14, 15, 16 or 17 of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6.

In some embodiments, the methods described herein comprise detecting an amount of expression of each of TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6.

In some embodiments, detecting an amount of expression of one or more of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, HOXC6, and KLK3 comprises detecting an mRNA or an amount of its expression.

In some embodiments, detecting an amount of expression of one or more of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, HOXC6, and KLK3 comprises detecting a cDNA reverse transcribed from an mRNA of the one or more genes.

In some embodiments, the methods described herein comprise detecting an amount of expression of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 of genes ACSM1, AMACR, AR, COL9A2, CRISP3, CST2, DLX1, ETV1, F5, GDF15, GLYATL1, GOLM1, GRIN3A, LINC00993, LRRN1, MIPEP, MS4A8, MYO6, PCA3.1, PDLIM5, PEX10, PLA1A, PLA2G7, PRCAT47, SPINK1, TDO2, TK1, TMEFF2, TRGV9.1, and VSTM2L.

In some embodiments, the methods described herein comprise detecting an amount of expression of each of genes ACSM1, AMACR, AR, COL9A2, CRISP3, CST2, DLX1, ETV1, F5, GDF15, GLYATL1, GOLM1, GRIN3A, LINC00993, LRRN1, MIPEP, MS4A8, MYO6, PCA3.1, PDLIM5, PEX10, PLA1A, PLA2G7, PRCAT47, SPINK1, TDO2, TK1, TMEFF2, TRGV9.1, and VSTM2L.

In some embodiments, detecting an amount of expression of one or more of genes ACSM1, AMACR, AR, COL9A2, CRISP3, CST2, DLX1, ETV1, F5, GDF15, GLYATL1, GOLM1, GRIN3A, LINC0093, LRRN1, MIPEP, MS4A8, MYO6, PCA3.1, PDLIM5, PEX10, PLA1A, PLA2G7, PRCAT47, SPINK1, TDO2, TK1, TMEFF2, TRGV9.1, and VSTM2L comprises detecting an RNA expressed by the one or more of the genes or an amount of the RNA's expression In some embodiments, the RNA is mRNA. In some embodiments, the RNA is miRNA. In some embodiments, the RNA is siRNA. In some embodiments, the RNA is circular RNA. In some embodiments, the RNA is lncRNA. In some embodiments, the RNA is piRNA. In some embodiments, the RNA is rRNA. In some embodiments, the RNA is tRNA. In some embodiments, the RNA is hnRNA. In some embodiments, the RNA is ncRNA.

In some embodiments, detecting an amount of expression of one or more of genes ACSM1, AMACR, AR, COL9A2, CRISP3, CST2, DLX1, ETV1, F5, GDF15, GLYATL1, GOLM1, GRIN3A, LINC0093, LRRN1, MIPEP, MS4A8, MYO6, PCA3.1, PDLIM5, PEX10, PLA1A, PLA2G7, PRCAT47, SPINK1, TDO2, TK1, TMEFF2, TRGV9.1, and VSTM2L comprises detecting a cDNA reverse transcribed from an RNA expressed by the one or more of the genes or an amount of the cDNA. In some embodiments, the RNA is mRNA. In some embodiments, the RNA is miRNA. In some embodiments, the RNA is siRNA. In some embodiments, the RNA is circular RNA. In some embodiments, the RNA is lncRNA. In some embodiments, the RNA is piRNA. In some embodiments, the RNA is rRNA. In some embodiments, the RNA is tRNA. In some embodiments, the RNA is hnRNA. In some embodiments, the RNA is ncRNA.

In some embodiments, the gene is a kidney cancer gene. Illustrative kidney cancer genes are described in Table 2 below.

TABLE 2

| | | HGNC | | Chromosome | SEQ |
|---|---|---|---|---|---|
| Gene | Gene Synonym | ID | Ensembl ID | Location | ID NO: |
| BMPR1B | ALK6; CDw293 | 1077 | ENSG00000138696 | 4q22.3 | 55 |
| CDH2 | CDHN; CD325 | 1759 | ENSG00000170558 | 18q12.1 | 56 |
| COL7A1 | Not applicable | 2214 | ENSG00000114270 | 3p21.31 | 57 |
| FGFR2 | CEK3; TK14; | 3689 | ENSG00000066468 | 10q26.13 | 58 |
| | TK25; ECT1; | | | | |
| | K-SAM; CD332 | | | | |
| HDHD3 | MGC12904 | 28171 | ENSG00000119431 | 9q32 | 59 |
| ICAM1 | BB2; CD54 | 5344 | ENSG00000090339 | 19p13.2 | 60 |
| JCAD | KIAA1462 | 29283 | ENSG00000165757 | 10p11.23 | 61 |
| PFKFB4 | Not applicable | 8875 | ENSG00000114268 | 3p21.31 | 62 |
| MIR15A | hsa-mir-15a | 31543 | ENSG00000283785 | 13q14.2 | 63 |
| MIR150 | hsa-mir-150 | 31537 | ENSG00000207782 | 19q13.33 | 64 |
| EGFL7 | ZNEU1 | 20594 | ENSG00000308874 | 9q34.3 | 65 |
| MIR210 | hsa-mir-210 | 31587 | ENSG00000199038 | 11p15.5 | 66 |
| MIRLET7A1 | hsa-let-7a-1 | 31476 | ENSG00000199165 | 9q22.32 | 67 |
| MIRLET7A2 | hsa-let-7a-2 | 31477 | ENSG00000198975 | 11q24.1 | 68 |
| MIRLET7A3 | hsa-let-7a-3 | 31478 | ENSG00000283990 | 22q13.31 | 69 |
| MIRLET7B | hsa-let-7b | 31479 | ENSG00000284520 | 22q13.31 | 70 |
| MIRLET7C | hsa-let-7c | 31480 | ENSG00000199030 | 21q21.1 | 71 |
| MIRLET7D | hsa-let-7d | 31481 | ENSG00000199133 | 9q22.32 | 72 |
| MIRLET7G | hsa-let-7g | 31485 | ENSG00000199150 | 3p21.2 | 73 |
| MIR30C1 | hsa-mir-30c-1 | 31626 | ENSG00000207962 | 1p34.2 | 74 |
| MIR200A | hsa-miR-200a | 31578 | ENSG00000207607 | 1p36.33 | 75 |
| MIR34A | hsa-mir-34a | 31635 | ENSG00000284357 | 1p36.22 | 76 |
| MIR365A | hsa-mir-365-1 | 33692 | ENSG00000199130 | 16p13.12 | 77 |
| MIR205 | hsa-mir-205 | 31583 | ENSG00000284485 | 1q32.2 | 78 |
| MIR497 | hsa-mir-497 | 32088 | ENSG00000284027 | 17p13.1 | 79 |
| EGLN3 | PHD3; HIFPH3 | 14661 | ENSG00000129521 | 14q13.1 | 80 |
| SOD2 | GC1; IPOB; | 11180 | ENSG00000291237 | 6q25.3 | 81 |
| | MnSOD; GClnc1; | | | | |
| | lncRNA-GC1 | | | | |

In some embodiments, the kidney cancer gene is BMPR1B, CDH2, COL7A1, FGFR2, HDHD3, ICAM1, JCAD, PFKFB4, MIR15A, MIR150, EGFL7, MIR210, MIRLET7A1, MIRLET7A2, MIRLET7A3, MIRLET7B, MIRLET7C, MIRLET7D, MIRLET7G, MIR30C1, MIR200A, MIR34A, MIR365A, MIR205, MIR497, EGLN3, or SOD2.

In some embodiments, the methods described herein comprise detecting an amount of expression of one or more of genes BMPR1B, CDH2, COL7A1, FGFR2, HDHD3, ICAM1, JCAD, PFKFB4, MIR15A, MIR150, EGFL7, MIR210, MIRLET7A1, MIRLET7A2, MIRLET7A3, MIRLET7B, MIRLET7C, MIRLET7D, MIRLET7G, MIR30C1, MIR200A, MIR34A, MIR365A, MIR205, MIR497, EGLN3, and SOD2.

In some embodiments, the methods described herein comprise detecting an amount of expression of 1, 2, 3, 4, 5, 6, 7, 8, 9, 10, 11, 12, 13, 14, 15, 16, 17, 18, 19, 20, 21, 22, 23, 24, 25, 26, 27, or 28 of genes BMPR1B, CDH2, COL7A1, FGFR2, HDHD3, ICAM1, JCAD, PFKFB4, MIR15A, MIR150, EGFL7, MIR210, MIRLET7A1, MIRLET7A2, MIRLET7A3, MIRLET7B, MIRLET7C, MIRLET7D, MIRLET7G, MIR30C1, MIR200A, MIR34A, MIR365A, MIR205, MIR497, EGLN3, and SOD2.

In some embodiments, the methods described herein comprise detecting an amount of expression of each of genes BMPR1B, CDH2, COL7A1, FGFR2, HDHD3, ICAM1, JCAD, PFKFB4, MIR15A, MIR150, EGFL7, MIR210, MIRLET7A1, MIRLET7A2, MIRLET7A3, MIRLET7B, MIRLET7C, MIRLET7D, MIRLET7G, MIR30C1, MIR200A, MIR34A, MIR365A, MIR205, MIR497, EGLN3, and SOD2.

In some embodiments, detecting an amount of expression of one or more of genes BMPR1B, CDH2, COL7A1, FGFR2, HDHD3, ICAM1, JCAD, PFKFB4, MIR15A, MIR150, EGFL7, MIR210, MIRLET7A1, MIRLET7A2, MIRLET7A3, MIRLET7B, MIRLET7C, MIRLET7D, MIRLET7G, MIR30C1, MIR200A, MIR34A, MIR365A, MIR205, MIR497, EGLN3, and SOD2 comprises detecting an RNA expressed by the one or more of the genes or an amount of the RNA's expression. In some embodiments, the RNA is mRNA. In some embodiments, the RNA is miRNA. In some embodiments, the RNA is siRNA. In some embodiments, the RNA is circular RNA. In some embodiments, the RNA is long non-coding RNA. In some embodiments, the RNA is piRNA. In some embodiments, the RNA is rRNA. In some embodiments, the RNA is tRNA. In some embodiments, the RNA is hnRNA. In some embodiments, the RNA is ncRNA.

In some embodiments, detecting an amount of expression of one or more of genes BMPR1B, CDH2, COL7A1, FGFR2, HDHD3, ICAM1, JCAD, PFKFB4, MIR15A, MIR150, EGFL7, MIR210, MIRLET7A1, MIRLET7A2, MIRLET7A3, MIRLET7B, MIRLET7C, MIRLET7D, MIRLET7G, MIR30C1, MIR200A, MIR34A, MIR365A, MIR205, MIR497, EGLN3, and SOD2 comprises detecting cDNA reversed transcribed from an RNA expressed by the one or more of the genes or an amount of the cDNA. In some embodiments, the RNA is mRNA. In some embodiments, the RNA is miRNA. In some embodiments, the RNA is siRNA. In some embodiments, the RNA is circular RNA. In some embodiments, the RNA is long non-coding RNA. In some embodiments, the RNA is piRNA. In some embodiments, the RNA is rRNA. In some embodiments, the RNA is tRNA. In some embodiments, the RNA is hnRNA. In some embodiments, the RNA is ncRNA.

In some embodiments, the gene is a bladder cancer gene. Illustrative bladder cancer genes are described in Table 3 below.

TABLE 3

| | | HGNC | | Chromosome | SEQ |
|---|---|---|---|---|---|
| Gene | Aliases | ID | Ensembl ID | Location | ID NO: |
| ABL1 | JTK7; c-ABL; p150 | 76 | ENSG00000097007 | 9q34.12 | 82 |
| ANXA10 | ANX14 | 534 | ENSG00000109511 | 4q32.3 | 83 |
| UPK1B | TSPAN20 | 12578 | ENSG00000114638 | 3q13.32 | 84 |
| CRH | CRF; CRH1 | 2355 | ENSG00000147571 | 8q13.1 | 85 |
| IGF2 | FLJ44734; IGF-II | 5466 | ENSG00000167244 | 11p15.5 | 86 |
| HOXA13 | Not applicable | 5102 | ENSG00000106031 | 7p15.2 | 87 |
| MDK | MK; FLJ27379 | 6972 | ENSG00000110492 | 11p11.2 | 88 |
| CXCR2 | CMKAR2; CD182 | 6027 | ENSG00000180871 | 2q35 | 89 |
| IGFBP5 | Not applicable | 5474 | ENSG00000115461 | 2q35 | 90 |
| ROBO1 | DUTT1; FLJ21882; | 10249 | ENSG00000169855 | 3p12.3 | 91 |
| | SAX3 | | | | |
| WNT5A | Not applicable | 12784 | ENSG00000114251 | 3p14.3 | 92 |
| CDC42BPB | MRCKB; KIAA1124 | 1738 | ENSG00000198752 | 14q32.32 | 93 |
| KIFC3 | Not applicable | 6326 | ENSG00000140859 | 16q21 | 94 |
| KRT20 | CK20; K20; | 20412 | ENSG00000171431 | 17q21.2 | 95 |
| | MGC35423 | | | | |
| LCN2 | NGAL; 24p3 | 6526 | ENSG00000148346 | 9q34.11 | 96 |
| MAGEA3 | HYPD; HIP8; | 6801 | ENSG00000221867 | Xq28 | 97 |
| | MGC14613; CT1.3 | | | | |
| RPS21 | S21; eS21 | 10409 | ENSG00000171858 | 20q13.33 | 98 |
| SLC1A6 | EAAT4 | 10944 | ENSG00000105143 | 19p13.12 | 99 |
| XIAP | hILP; ILP-1 | 592 | ENSG00000101966 | Xq25 | 100 |
| CDK1 | CDC2; CDC28A | 1722 | ENSG00000170312 | 10q21.2 | 101 |
| PRSS1 | Not applicable | 9475 | ENSG00000204983 | 7q34 | 102 |
| CASP14 | MICE; MGC119078; | 1502 | ENSG00000105141 | 19p13.12 | 103 |
| | MGC119079; | | | | |
| | caspase-14 | | | | |
| KLHDC7B | MGC16635 | 25145 | ENSG00000130487 | 22q13.33 | 104 |
| PCAT-1 | PCAT-1; PCA1; | 43022 | ENSG00000253438 | 8q24.21 | 105 |
| | PiHL | | | | |
| ANRIL | CDKN2B-AS1; | 34341 | ENSG00000240498 | 9p21.3 | 106 |
| | RP11-145E5.4; | | | | |
| | NCRNA00089; | | | | |
| | p15AS; CDKN2B- | | | | |
| | AS; PCAT12 | | | | |
| MKLN1-AS | Not applicable | 40374 | ENSG00000236753 | 7q32.3 | 107 |
| TALAM1 | Not applicable | 54476 | ENSG00000289740 | 11q13.1 | 108 |
| TTN-AS1 | Not applicable | 44124 | ENSG00000237298 | 2q31.2 | 109 |
| UCA1 | LINC00178; CUDR; | 37126 | ENSG00000214049 | 19p13.12 | 110 |
| | UCAT1; onco- | | | | |
| | lncRNA-36 | | | | |
| LNMAT2 | LINC00858; | 27276 | ENSG00000229404 | 10q23.1 | 111 |
| | CRCAL-2 | | | | |
| BCYRN1 | BC200; BC200a; | 1022 | ENSG00000236824 | 2p21 | 112 |
| | NCRNA00004; | | | | |
| | LINC00004 | | | | |
| GAS5 | SNHG2; | 16355 | ENSG00000234741 | 1q25.1 | 113 |
| | NCRNA00030 | | | | |
| CD24 | CD24A | 1645 | ENSG00000272398 | 6q21 | 114 |
| TOP2A | TOP2alpha; TOPIIA | 11989 | ENSG00000131747 | 17q21.2 | 115 |
| IQGAP3 | Not applicable | 20669 | ENSG00000183856 | 1q22 | 116 |
| UBE2C | UBCH10 | 15937 | ENSG00000175063 | 20q13.12 | 117 |
| IGFBP5 | Not applicable | 5474 | ENSG00000115461 | 2q35 | 118 |
| BIRC5 | EPR-1; survivin | 593 | ENSG00000089685 | 17q25.3 | 119 |
| MIR29B2 | miR-29b-3p; | 31620 | ENSG00000284203 | 1q32.2 | 120 |
| | hsa-mir-29b-2 | | | | |
| MIR31 | hsa-mir-31 | 31630 | ENSG00000199177 | 9p21.3 | 121 |
| MIR141 | hsa-mir-141 | 31528 | ENSG00000207708 | 12p13.31 | 122 |
| MIR34B | hsa-mir-34b | 31636 | ENSG00000207811 | 11q23.1 | 123 |
| MIR10B | hsa-mir-10b | 31498 | ENSG00000207744 | 2q31.1 | 124 |
| MIR103A1 | hsa-mir-103-1; | 31490 | ENSG00000199035 | 5q34 | 125 |
| | hsa-mir-103a-1 | | | | |
| MIR103A2 | hsa-mir-103-2; | 31491 | ENSG00000199024 | 20p13 | 126 |
| | hsa-mir-103a-2 | | | | |
| MIR103B1 | hsa-mir-103-1-as; | 35384 | ENSG00000283612 | 5q34 | 127 |
| | hsa-mir-103b-1 | | | | |
| MIR145 | hsa-mir-145 | 31532 | ENSG00000276365 | 5q32 | 128 |
| MIR182 | hsa-mir-182 | 31553 | ENSG00000207990 | 7q32.2 | 129 |
| MIR205HG | miR-205HG; | 43562 | ENSG00000230937 | 1q32.2 | 130 |
| | LEADR; miPEP205; | | | | |
| | LINC00510 | | | | |
| MIR20A | hsa-mir-20; | 31577 | ENSG00000283762 | 13q31.3 | 131 |
| | hsa-mir-20a | | | | |
| MIR92A2 | hsa-mir-92-2; | 31644 | ENSG00000284538 | Xq26.2 | 132 |
| | hsa-mir-92a-2 | | | | |
| MIR17 | hsa-mir-17 | 31547 | ENSG00000284536 | 13q31.3 | 133 |
| MALAT1 | HCN; NEAT2; | 29665 | ENSG00000251562 | 11q13.1 | 134 |
| | PRO1073; MALAT- | | | | |
| | 1; NCRNA00047; | | | | |
| | LINC00047; | | | | |
| | mascRNA | | | | |
| SPRY4-IT1 | SPRIGHTLY | 42394 | | 5q31.3 | 135 |
| HOTAIR | HOXC-AS4; | 33510 | ENSG00000228630 | 12q13.13 | 136 |
| | HOXC11-AS1; | | | | |
| | NCRNA00072 | | | | |
| HYMAI | NCRNA00020 | 5326 | ENSG00000283122 | 6q24.2 | 137 |
| CCDC148-AS1 | Not applicable | 44134 | ENSG00000227480 | 2q24.1 | 138 |
| CCDC86-AS1 | RP5-1148A21.3; | 56314 | ENSG00000256813 | 11q12.2 | 139 |
| | IncRP5 | | | | |
| KDM4C | GASC1; KIAA0780; | 17071 | ENSG00000107077 | 9p24.1 | 140 |
| | TDRD14C | | | | |
| ITCH | AIP4 | 13890 | ENSG00000078747 | 20q11.22 | 141 |
| ACVR2A | ACTRII | 173 | ENSG00000121989 | 2q22.3-q23.1 | 142 |
| MTDH | LYRIC; 3D3; AEG-1 | 29608 | ENSG00000147649 | 8q22.1 | 143 |
| ATIC | PURH; AICARFT; | 794 | ENSG00000138363 | 2q35 | 144 |
| | IMPCHASE | | | | |
| TAF4B | TAFII105 | 11538 | ENSG00000141384 | 18q11.2 | 145 |
| RBM3 | IS1-RNPL | 9900 | ENSG00000102317 | Xp11.23 | 146 |
| GLI3 | PAP-A; PAPA; | 4319 | ENSG00000106571 | 7p14.1 | 147 |
| | PAPA1; PAPB; | | | | |
| | ACLS; PPDIV | | | | |

In some embodiments, the bladder cancer gene is ABL1, ANXA10, UPK1B, CRH, IGF2, HOXA13, MDK, CXCR2, IGFBP5, ROBO1, WNT5A, CDC42BPB, KIFC3, KRT20, LCN2, MAGEA3, RPS21, SLC1A6, XIAP, CDK1, PRSS1, CASP14, KLHDC7B, ANRIL, MKLN1-AS, TALAM1, TTN-AS1, UCA1, LNMAT2, BCYRNI, GAS5, CD24, TOP2A, IQGAP3, UBE2C, IGFBP5, BIRC5, MIR29B2, MIR31, MIR141, MIR34B, MIR10B, MIR103A1, MIR103A2, MIR103B1, MIR145, MIR182, MIR205HG, MIR20A, MIR92A2, MIR17, MALAT1, PCAT-1, SPRY4-IT1, HOTAIR, HYMAI, CCDC148-AS1, CCDC86-AS1, KDM4C, ITCH, ACVR2A, MTDH, ATIC, TAF4B, RBM3, or GLI3.

In some embodiments, the methods described herein comprise detecting an amount of expression of one or more of genes ABL1, ANXA10, UPK1B, CRH, IGF2, HOXA13, MDK, CXCR2, IGFBP5, ROBO1, WNT5A, CDC42BPB, KIFC3, KRT20, LCN2, MAGEA3, RPS21, SLC1A6, XIAP, CDK1, PRSS1, CASP14, KLHDC7B, ANRIL, MKLN1-AS, TALAM1, TTN-AS1, UCA1, LNMAT2, BCYRNI, GAS5, CD24, TOP2A, IQGAP3, UBE2C, IGFBP5, BIRC5, MIR29B2, MIR31, MIR141, MIR34B, MIR10B, MIR103A1, MIR103A2, MIR103B1, MIR145, MIR182, MIR205HG, MIR20A, MIR92A2, MIR17, MALAT1, PCAT-1, SPRY4-IT1, HOTAIR, HYMAI, CCDC148-AS1, CCDC86-AS1, KDM4C, ITCH, ACVR2A, MTDH, ATIC, TAF4B, RBM3, and GLI3.

In some embodiments, the methods described herein comprise detecting an amount of expression of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 of genes ABL1, ANXA10, UPK1B, CRH, IGF2, HOXA13, MDK, CXCR2, IGFBP5, ROBO1, WNT5A, CDC42BPB, KIFC3, KRT20, LCN2, MAGEA3, RPS21, SLC1A6, XIAP, CDK1, PRSS1, CASP14, KLHDC7B, ANRIL, MKLN1-AS, TALAM1, TTN-AS1, UCA1, LNMAT2, BCYRNI, GAS5, CD24, TOP2A, IQGAP3, UBE2C, IGFBP5, BIRC5, MIR29B2, MIR31, MIR141, MIR34B, MIR10B, MIR103A1, MIR103A2, MIR103B1, MIR145, MIR182, MIR205HG, MIR20A, MIR92A2, MIR17, MALAT1, PCAT-1, SPRY4-IT1, HOTAIR, HYMAI, CCDC148-AS1, CCDC86-AS1, KDM4C, ITCH, ACVR2A, MTDH, ATIC, TAF4B, RBM3, and GLI3.

In some embodiments, the methods described herein comprise detecting an amount of expression of each of genes ABL1, ANXA10, UPK1B, CRH, IGF2, HOXA13, MDK, CXCR2, IGFBP5, ROBO1, WNT5A, CDC42BPB, KIFC3, KRT20, LCN2, MAGEA3, RPS21, SLC1A6, XIAP, CDK1, PRSS1, CASP14, KLHDC7B, ANRIL, MKLN1-AS, TALAM1, TTN-AS1, UCA1, LNMAT2, BCYRNI, GAS5, CD24, TOP2A, IQGAP3, UBE2C, IGFBP5, BIRC5, MIR29B2, MIR31, MIR141, MIR34B, MIR10B, MIR103A1, MIR103A2, MIR103B1, MIR145, MIR182, MIR205HG, MIR20A, MIR92A2, MIR17, MALAT1, PCAT-1, SPRY4-IT1, HOTAIR, HYMAI, CCDC148-AS1, CCDC86-AS1, KDM4C, ITCH, ACVR2A, MTDH, ATIC, TAF4B, RBM3, and GLI3.

In some embodiments, detecting an amount of expression of one or more of genes ABL1, ANXA10, UPK1B, CRH, IGF2, HOXA13, MDK, CXCR2, IGFBP5, ROBO1, WNT5A, CDC42BPB, KIFC3, KRT20, LCN2, MAGEA3, RPS21, SLC1A6, XIAP, CDK1, PRSS1, CASP14, KLHDC7B, ANRIL, MKLN1-AS, TALAM1, TTN-AS1, UCA1, LNMAT2, BCYRNI, GAS5, CD24, TOP2A, IQGAP3, UBE2C, IGFBP5, BIRC5, MIR29B2, MIR31, MIR141, MIR34B, MIR10B, MIR103A1, MIR103A2, MIR103B1, MIR145, MIR182, MIR205HG, MIR20A, MIR92A2, MIR17, MALAT1, PCAT-1, SPRY4-IT1, HOTAIR, HYMAI, CCDC148-AS1, CCDC86-AS1, KDM4C, ITCH, ACVR2A, MTDH, ATIC, TAF4B, RBM3, and GLI3 comprises detecting an RNA expressed by the one or more of the genes or an amount of the RNA's expression. In some embodiments, the RNA is mRNA. In some embodiments, the RNA is micro-RNA. In some embodiments, the RNA is siRNA. In some embodiments, the RNA is circular RNA. In some embodiments, the RNA is long non-coding RNA. In some embodiments, the RNA is piRNA. In some embodiments, the RNA is rRNA. In some embodiments, the RNA is tRNA. In some embodiments, the RNA is hnRNA. In some embodiments, the RNA is ncRNA.

In some embodiments, detecting an amount of expression of one or more of genes ABL1, ANXA10, UPK1B, CRH, IGF2, HOXA13, MDK, CXCR2, IGFBP5, ROBO1, WNT5A, CDC42BPB, KIFC3, KRT20, LCN2, MAGEA3, RPS21, SLC1A6, XIAP, CDK1, PRSS1, CASP14, KLHDC7B, ANRIL, MKLN1-AS, TALAM1, TTN-AS1, UCA1, LNMAT2, BCYRNI, GAS5, CD24, TOP2A, IQGAP3, UBE2C, IGFBP5, BIRC5, MIR29B2, MIR31, MIR141, MIR34B, MIR10B, MIR103A1, MIR103A2, MIR103B1, MIR145, MIR182, MIR205HG, MIR20A, MIR92A2, MIR17, MALAT1, PCAT-1, SPRY4-IT1, HOTAIR, HYMAI, CCDC148-AS1, CCDC86-AS1, KDM4C, ITCH, ACVR2A, MTDH, ATIC, TAF4B, RBM3, and GLI3 comprises detecting cDNA reversed transcribed from an RNA expressed by the one or more of the genes or an amount of the cDNA. In some embodiments, the RNA is mRNA. In some embodiments, the RNA is micro-RNA. In some embodiments, the RNA is siRNA. In some embodiments, the RNA is circular RNA. In some embodiments, the RNA is a long non-coding RNA. In some embodiments, the RNA is piRNA. In some embodiments, the RNA is rRNA. In some embodiments, the RNA is tRNA. In some embodiments, the RNA is hnRNA. In some embodiments, the RNA is ncRNA.

In some embodiments, the gene is a gene of a pathogen, e.g., a sexually transmitted infection (STI) pathogen or a urinary tract infection (UTI) pathogen. In some embodiments, the pathogen is a bacterium. In some embodiments, the bacterium is the bacterium is of the genus Escherichia, Klebsiella, Proteus, Enterococcus, Staphylococcus, Pseudomonas, Staphylococcus, Streptococcus, or Staphylococcus. In some embodiments, the bacterium is Escherichia coli, Klebsiella pneumoniae, Proteus mirabilis, Enterococcus faecalis, Staphylococcus saprophyticus, Pseudomonas aeruginosa, Staphylococcus saprophyticus, Streptococcus agalactiae or Staphylococcus aureus.

In some embodiments, the pathogen is a yeast. In some embodiments, the yeast is of the genus Candida. In some embodiments, the yeast is Candida albicans.

In some embodiments, the gene is a gene of a pathogen and the gene is 16S rRNA. In some embodiments, the gene is of a pathogen and the gene is 23S rRNA. In some embodiments, the gene is of a pathogen and the gene is 26S rRNA.

In some embodiments, the gene is from the subject's immune response to the pathogen, e.g., a gene of the subject's B cell or T cell engaged in the immune response.

### Detecting Expression of Genes

In some embodiments, detecting the extracted RNA comprises detecting an amount of expression of the subject's gene. In some embodiments, detecting the extracted RNA comprises detecting an amount of expression of RNA transcribed from the subject's gene. In some embodiments, detecting the extracted RNA comprises detecting the amount of mRNA transcribed from the gene. In some embodiments, detecting the extracted RNA comprises reverse transcribing the extracted RNA to complementary DNA (cDNA) using reverse transcriptase and detecting the cDNA.

The amount of expression of a subject's gene can be detected using any of a variety of nucleic acid techniques. Suitable techniques include, but are not limited to: nucleic acid sequencing; nucleic acid hybridization; and nucleic acid amplification. In some embodiments, detecting the extracted RNA comprises performing: reverse transcription-quantitative polymerase chain reaction (RT-qPCR); RNA-sequencing; reverse transcription digital PCR (RT-dPCR); quantitative PCR (qPCR); digital PCR (dPCR); microarray analysis; Northern Blot; UV spectroscopy; or fluorometry.

The amount of gene expression can be detected using a Second Generation (i.e., Next Generation or Next-Gen), Third Generation (i.e., Next-Next-Gen), or Fourth Generation (i.e., N3-Gen) sequencing technology including, but not limited to, pyrosequencing, sequencing-by-ligation, single molecule sequencing, sequence-by-synthesis (SBS), semiconductor sequencing, massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008). Those of skill in the art will recognize that because RNA is less stable in the cell and more prone to nuclease attack, the extracted RNA can be reverse transcribed to complementary DNA (cDNA) before sequencing. A number of DNA sequencing techniques are suitable for gene expression detection, including fluorescence-based sequencing methodologies (See, e.g., Birren et al., Genome Analysis: Analyzing DNA, 1, Cold Spring Harbor, N.Y. In some embodiments, the sequencing is automated sequencing techniques understood in the art. In some embodiments, the sequencing is parallel sequencing of partitioned amplicons (PCT Publication No: WO2006084132 to Kevin McKernan et al. In some embodiments, the sequencing is DNA sequencing by parallel oligonucleotide extension (See, e.g., U.S. Pat. No. 5,750,341 to Macevicz et al., and U.S. Pat. No. 6,306,597 to Macevicz et al. Additional examples of sequencing techniques include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803, the 454 picotiter pyrosequencing technology (Margulies et al., 2005 Nature 437, 376-380; US 20050130173, the Solexa single base addition technology (Bennett et al., 2005, Pharmacogenomics, 6, 373-382; U.S. Pat. Nos. 6,787,308; 6,833,246, the Lynx massively parallel signature sequencing technology (Brenner et al. (2000). Nat. Biotechnol. 18:630-634; U.S. Pat. Nos. 5,695,934; 5,714,330, and the Adessi PCR colony technology (Adessi et al. (2000). Nucleic Acid Res. 28, E87; WO 00018957.

Illustrative non-limiting examples of nucleic acid hybridization techniques include, but are not limited to, in situ hybridization (ISH), microarray, and Southern or Northern blot.

In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH can be used to measure and localize mRNAs and other transcripts (e.g., cancer markers) within tissue sections or whole mounts. Sample cells and tissues can be treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

Expression of each of the one or more genes of the present methods can be detected by conducting one or more hybridization reactions. The one or more hybridization reactions can comprise one or more hybridization arrays, hybridization reactions, hybridization chain reactions, isothermal hybridization reactions, nucleic acid hybridization reactions, or a combination thereof. The one or more hybridization arrays can comprise hybridization array genotyping, hybridization array proportional sensing, DNA hybridization arrays, macroarrays, microarrays, high-density oligonucleotide arrays, genomic hybridization arrays, comparative hybridization arrays, or a combination thereof.

Microarrays including, but not limited to, DNA microarrays (e.g., cDNA microarrays and oligonucleotide microarrays); protein microarrays; tissue microarrays; transfection or cell microarrays; chemical compound microarrays; and antibody microarrays, can optionally be employed. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes or transcripts by comparing gene expression in diseased and normal cells. Microarrays can be fabricated using a variety of technologies, including but not limited to: printing with fine-pointed pins onto glass slides; photolithography using pre-made masks; photolithography using dynamic micromirror devices; ink-jet printing; or, electrochemistry on microelectrode arrays.

Detection of an amount of expression of the one or more genes of the present methods can comprise conducting one or more amplification reactions. Nucleic acids can be amplified prior to or simultaneous with detection. Conducting one or more amplification reactions can comprise one or more PCR-based amplifications, non-PCR based amplifications, or a combination thereof. Illustrative non-limiting examples of nucleic acid amplification techniques include, but are not limited to, polymerase chain reaction (PCR), quantitative polymerase chain reaction (qPCR), digital polymerase chain reaction (dPCR), reverse transcription-polymerase chain reaction (RT-PCR), nested PCR, linear amplification, multiple displacement amplification (MDA), real-time SDA, rolling circle amplification, circle-to-circle amplification transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to complementary DNA (cDNA) prior to amplification by qPCR or dPCR, whereas other amplification techniques directly amplify RNA (e.g., TMA and NASBA).

The polymerase chain reaction (U.S. Pat. Nos. 4,683,195, 4,683,202, 4,800,159 and 4,965,188), commonly referred to as PCR, uses multiple cycles of denaturation, annealing of primer pairs to opposite strands, and primer extension to exponentially increase copy numbers of a target nucleic acid sequence. In a variation called reverse transcription-polymerase chain reaction (RT-PCR), reverse transcriptase (RT) is used to make a complementary DNA (cDNA) from mRNA, and the cDNA is then amplified by PCR to produce multiple copies of DNA. In some embodiments, the RT-PCR is RT-dPCR. In some embodiments, the RT-PCR is RT-qPCR. For other various permutations of PCR see, e.g., U.S. Pat. Nos. 4,683,195, 4,683,202 and 4,800,159; Mullis et al., Meth. Enzymol. 155: 335 (1987); and Murakawa et al., DNA 7: 287 (1988). In some embodiments, the reverse transcriptase is Moloney murine leukemia virus (M-MLV) reverse transcriptase.

Transcription mediated amplification (U.S. Pat. Nos. 5,480,784 and 5,399,491), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518. In a variation described in U.S. Publ. No. 20060046265, TMA optionally incorporates the use of blocking moieties, terminating moieties, and other modifying moieties to improve TMA process sensitivity and accuracy.

The ligase chain reaction (Weiss, R., Science 254: 1292 (1991), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89: 392-396 (1992); U.S. Pat. Nos. 5,270,184 and 5,455,166), commonly referred to as SDA, uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Patent No. 0 684 315).

Other amplification methods include, for example: nucleic acid sequence-based amplification (U.S. Pat. No. 5,130,238), commonly referred to as NASBA; one that uses an RNA replicase to amplify the probe molecule itself (Lizardi et al., BioTechnol. 6: 1197 (1988)), commonly referred to as Q3 replicase; a transcription-based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990)). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, DC (1993)).

In some embodiments, amplification methods are quantitative PCR methods (qPCR), also known as real time PCR (RT-PCR). qPCR is a laboratory technique of molecular biology based on the polymerase chain reaction (PCR). It monitors the amplification of a targeted DNA molecule during the PCR (i.e., in real time), not at its end, as in conventional PCR. qPCR can be used quantitatively or semi-quantitatively (i.e., above/below a certain amount of DNA molecules). Two common methods for the detection of PCR products in qPCR are (1) non-specific fluorescent dyes that intercalate with any double-stranded DNA and (2) sequence-specific DNA probes consisting of oligonucleotides that are labelled with a fluorescent reporter, which permits detection only after hybridization of the probe with its complementary sequence.

In some embodiments, detecting an amount of gene expression comprises detecting RNA or an amount of its expression. In some embodiments, the amount of RNA is detected by reverse transcribing the RNA to cDNA and detecting the cDNA using qPCR analysis, which provides a Crt (cycle threshold) value for each cDNA detected. In a qPCR assay a positive reaction is detected by accumulation of a fluorescent signal. The Crt value is defined as the number of cycles required for the fluorescent signal to cross the threshold (i.e., to exceed the background level). Crt values are inversely proportional to the amount of target nucleic acid in the sample (i.e., the lower the Crt value the greater the amount of mRNA in the sample). In some embodiments, the Crt value is about 35 cycles, about 34 cycles, about 33 cycles, about 32 cycles, about 31 cycles, about 30 cycles, about 29 cycles, about 28 cycles, about 27 cycles, about 26 cycles, about 25 cycles, about 24 cycles, about 23 cycles, about 22 cycles, about 21 cycles, about 20 cycles, about 19 cycles, about 18 cycles, about 17 cycles, about 16 cycles, about 15 cycles, or lower using the RNA extraction methods described herein. In some embodiments, the Crt value is about 35 cycles or lower using the RNA extraction methods described herein. In some embodiments, the Crt value is about 30 cycles or lower. In some embodiments, the Crt value is about 25 cycles or lower. In some embodiments, the Crt value is about 20 cycles or lower. In some embodiments, the gene is one or more of the of the prostate cancer, kidney cancer, bladder cancer, STI, UTI or yeast genes disclosed herein. In some embodiments, the gene is 16S rRNA, 23S rRNA or 26S rRNA. In some embodiments, the gene is of the subject's B cell or T cell engaged in an immune response.

In some embodiments, the RNA is mRNA. In some embodiments, the RNA is miRNA. In some embodiments, the RNA is siRNA. In some embodiments, the RNA is long non-coding RNA. In some embodiments, the RNA is circular RNA. In some embodiments, the RNA is piRNA. In some embodiments, the RNA is rRNA. In some embodiments, the RNA is tRNA. In some embodiments, the RNA is hnRNA. In some embodiments, the RNA is ncRNA.

In some embodiments, the amount of extracted RNA or cDNA detectable according to the RNA extraction methods described herein is significantly greater than that detectable from a method that does not comprise admixing whole urine and a solid support comprising silicon carbide. In some embodiments, the amount of extracted RNA or cDNA detectable according to the RNA extraction methods described herein about 1.5-fold, about 2-fold, about 2.5-fold, about 3-fold, about 4-fold, about 4.5-fold, about 5-fold, about 6-fold, about 7-fold, about 8-fold, about 9-fold, about 10-fold, about 20-fold, about 25-fold, about 30-fold, about 40-fold, about 50-fold, about 75-fold, or about 100-fold greater than the amount of extracted RNA or cDNA detectable according to a method that does not comprise admixing whole urine and a solid support comprising silicon carbide. In some embodiments, the amount of extracted RNA or cDNA detectable according to the RNA extraction methods described herein is about 100%, about 150%, about 175%, about 200%, about 250%, about 275%, about 300%, about 350%, about 375%, about 400%, about 450%, about 475%, about 500%, about 550%, about 575%, about 600%, about 650%, about 675%, about 700%, about 800%, about 900%, or about 1000% greater than the amount of extracted RNA or cDNA detectable according to a method that does not comprise admixing whole urine and a solid support comprising silicon carbide.

In some embodiments, RT-qPCR comprises transcribing cDNA from extracted RNA using a Moloney murine leukemia virus (M-MLV) reverse transcriptase. In some embodiments, the ratio of M-MLV reverse transcriptase to extracted RNA to elution buffer is about 1:about 11:about 100 (v/v/v), for example, about 1 μL of M-MLV reverse transcriptase to about 11 μL of extracted RNA in about 100 μL of elution buffer.

In some embodiments, the amount of expression of any one of the genes described herein is normalized to an amount of expression of a reference gene. In some embodiments, the amount of expression of mRNA is normalized to an amount of expression of mRNA of a reference gene.

Reference genes suitable for normalization are known to those of skill in the art and include, but are not limited to, KLK3, CYPB561A3, EEF1A2, GAPDH, HPN, KLK2, LBH, NUDT8, SPDEF, or TRGV. In some embodiments, the reference gene is KLK3.

Compositions that are useful for detecting an amount of gene expression (“detection compositions”) can comprise one or more antibodies, probes, amplification oligonucleotides or reagents.

Detection compositions can comprise 1 or more, 2 or more, 3 or more, or 4 or more antibodies, probes, pairs of probes, pairs of amplification oligonucleotide, or sequencing primers. The probes or primers can hybridize to 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 20 or more, or 21 or more target molecules. The target molecules may be RNA, DNA, cDNA, mRNA, miRNA, siRNA, long non-coding RNA, circular RNA, piRNA, rRNA, tRNA, hnRNA or ncRNA; a portion or fragment thereof; or a combination thereof. In some instances, at least a portion of the target molecules are cancer markers. The probes may hybridize to 1 or more, or 2 or more cancer markers disclosed herein. Typically, the probes or primers comprise a target specific sequence. The target specific sequence may be complementary to at least a portion of the target molecule. The target specific sequence may be at least about 50% or more, 55% or more, 60% or more, 65% or more, 70% or more, 75% or more, 80% or more, 85% or more, 90% or more, 95% or more, 97% or more, 98% or more, or 100% complementary to at least a portion of the target molecule. The target specific sequence can be at least about 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more nucleotides in length. In some instances, the target specific sequence is between about 8 to about 20 nucleotides, 10 to about 18 nucleotides, or 12 to about 16 nucleotides in length.

These detection compositions can comprise a plurality of probes or primers, wherein the two or more probes of the plurality of probes comprise identical target specific sequences. The detection compositions may comprise a plurality of probes, wherein the two or more probes of the plurality of probes comprise different target specific sequences.

The probes can further comprise a unique sequence. The unique sequence is noncomplementary to the target marker. The unique sequence may comprise a label, barcode, or unique identifier. The unique sequence may comprise a random sequence, nonrandom sequence, or a combination thereof. The unique sequence may be at least about 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 22 or more, 24 or more, 26 or more, 28 or more, 30 or more nucleotides in length. In some instances, the unique sequence is between about 8 to about 20 nucleotides, 10 to about 18 nucleotides, or 12 to about 16 nucleotides in length.

The probes can further comprise a universal sequence. The universal sequence may comprise a primer binding site. The universal sequence may enable detection of the target sequence. The universal sequence may enable amplification of the target sequence. The universal sequence may enable transcription or reverse transcription of the target sequence. The universal sequence may enable sequencing of the target sequence.

Detection compositions, such as for RNA-sequencing, comprising a probe or primer can be provided on a solid support. The solid support can comprise one or more beads, plates, solid surfaces, wells, chips, or a combination thereof. The beads can be magnetic, antibody coated, protein A crosslinked, protein G crosslinked, streptavidin coated, oligonucleotide conjugated, silica coated, or a combination thereof. Examples of beads include, but are not limited to, Ampure beads, AMPure XP beads, streptavidin beads, agarose beads, magnetic beads, DYNABEADS, MACS microbeads, antibody conjugated beads (e.g., anti-immunoglobulin microbead), protein A conjugated beads, protein G conjugated beads, protein A/G conjugated beads, protein L conjugated beads, oligo-dT conjugated beads, silica beads, silica-like beads, anti-biotin microbead, anti-fluorochrome microbead, and BCMAG Carboxy-Terminated Magnetic Beads.

The detection compositions can comprise one or more primers or primer pairs capable of amplifying target molecules, or fragments or subsequences or complements thereof. The nucleotide sequences of the target molecules may be provided in computer-readable media for in silico applications and as a basis for the design of appropriate primers for amplification of one or more target molecules.

Primers based on the nucleotide sequences of target molecules can be designed for use in amplification of the target molecules. For use in amplification reactions such as PCR, a pair of primers can be used. The exact composition of the primer sequences is not critical to the disclosure, but for most applications the primers may hybridize to specific sequences of the target molecules or the universal sequence of the probe under stringent conditions, particularly under conditions of high stringency, as known in the art. The pairs of primers are usually chosen so as to generate an amplification product of at least about 15 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 450 or more, 500 or more, 600 or more, 700 or more, 800 or more, 900 or more, or 1000 or more nucleotides. Algorithms for the selection of primer sequences are generally known and are commercially available. These primers may be used in standard quantitative or qualitative PCR-based assays to assess transcript expression levels of target molecules. Alternatively, these primers may be used in combination with probes, such as molecular beacons in amplifications using qPCR.

The nucleotide sequence of the entire length of the primer does not need to be derived from the target sequence. Thus, for example, the primer may comprise nucleotide sequences at the 5′ and/or 3′ termini that are not derived from the target molecule. Nucleotide sequences which are not derived from the nucleotide sequence of the target molecule may provide additional functionality to the primer. For example, they may provide a restriction enzyme recognition sequence or a “tag” that facilitates detection, isolation, purification or immobilization onto a solid support. Alternatively, the additional nucleotides may provide a self-complementary sequence that allows the primer to adopt a hairpin configuration. Such configurations may be necessary for certain primers, for example, molecular beacon and Scorpion primers, which can be used in solution hybridization techniques.

The probes or primers can incorporate moieties useful in detection, isolation, purification, or immobilization, if desired. Such moieties are well-known in the art (see, for example, Ausubel et al., (1997 & updates) Current Protocols in Molecular Biology, Wiley & Sons, New York) and are chosen such that the ability of the probe to hybridize with its target molecule is not affected.

Examples of suitable moieties are detectable labels, such as radioisotopes, fluorophores, chemiluminophores, enzymes, colloidal particles, and fluorescent microparticles, as well as antigens, antibodies, haptens, avidin/streptavidin, biotin, haptens, enzyme cofactors/substrates, enzymes, and the like.

A label can optionally be attached to or incorporated into a probe or primer to allow detection and/or quantitation of a target polynucleotide representing the target molecule of interest. The target polynucleotide may be the expressed target molecule RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different target molecules may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g., biotin-avidin or streptavidin). Many labels are commercially available in activated forms which can readily be used for such conjugation (for example through amine acylation), or labels may be attached through known or determinable conjugation schemes, many of which are known in the art.

Labels useful in the disclosure described herein include any substance which can be detected when bound to or incorporated into the target molecule. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a target polynucleotide may comprise a biotin-binding species, and an optically detectable label may be conjugated to biotin and then bound to the labeled target polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes (e.g., intercalating dyes) and fluorescent proteins (e.g., GFP or RFP).

### Subjects, Samples, and Kits

In some embodiments, the present methods comprise extracting RNA present in whole urine from a subject's urine sample. In some embodiments, the present methods comprise detecting an amount of expression of RNA extracted from whole urine from a subject's urine sample. In some embodiments, the RNA is mRNA. In some embodiments, the RNA is miRNA. In some embodiments, the RNA is siRNA. In some embodiments, the RNA is long non-coding RNA. In some emobimdents, the RNA is circular RNA. In some embodiments, the RNA is piRNA. In some embodiments, the RNA is rRNA. In some embodiments, the RNA is tRNA. In some embodiments, the RNA is hnRNA. In some embodiments, the RNA is ncRNA.

In some embodiments, the whole urine comprises first-catch urine. In some embodiments, the whole urine is first-catch urine.

The methods disclosed herein are useful for both male and female subjects where the gene is not a prostate cancer gene. In some embodiments, the subject is a human subject.

In some embodiments, the subject is male. In some embodiments, the male subject has a prostate. In some embodiments, the subject is prostate biopsy-naïve. In some embodiments, the subject is prostate biopsy-prior negative. In some embodiments, the subject is prostate biopsy-prior negative for Grade Group ≥2 prostate cancer. In some embodiments, the subject is a male subject, and the urine sample provided by the subject comprises prostatic fluid. In some embodiments, the subject is a male human having a prostate.

In some embodiments, a subject from whom a urine sample is obtained is selected by a skilled practitioner, e.g., internist, urologist or oncologist.

In some embodiments, the subject had a DRE within about 180 minutes before providing the urine sample. In some embodiments, the subject is a male human having a prostate and the male human having a prostate had a DRE within about 180 minutes before providing the urine sample.

In some embodiments, the subject did not have a DRE within about 180 minutes before providing the urine sample. As a DRE is invasive and uncomfortable, it is believed that the present methods' use of a urine sample provided by a subject that did not have a DRE within about 180 minutes before providing the urine sample can result in higher subject compliance with urine-based tests for prognosis and/or diagnosis, such as for prostate cancer prognosis and/or diagnosis. In some embodiments, the subject is a male human having a prostate and the male human having a prostate did not have a DRE within about 180 minutes before providing a urine sample.

In some embodiments, the subject has or is suspected of having cancer. In some embodiments, the subject has or is suspected of having prostate cancer. In some embodiments, the prostate cancer is Grade Group (GG)≥2 prostate cancer. In some embodiments, the prostate cancer is GG≥3 prostate cancer. In some embodiments, the prostate cancer is GG≥4 prostate cancer. In some embodiments, the prostate cancer is GG5 prostate cancer. In some embodiments, the prostate cancer has metastasized.

In some embodiments, the urine sample provided by a subject is provided outside a clinical setting. In some embodiments, the urine sample is provided directly by the subject, i.e., without the assistance of a clinician or medical service provider.

In some embodiments, the urine for use in the present methods comprises a nucleic acid, and the detecting comprises detecting an amount of the nucleic acid. In some embodiments, the urine for use in the present methods comprises RNA, and the detecting comprises detecting an amount of the RNA. In some embodiments, the urine for use in the present methods comprises mRNA, and the detecting comprises detecting an amount of the mRNA or cDNA reversed transcribed from the mRNA. In some embodiments, the urine for use in the present methods comprises miRNA, and the detecting comprises detecting an amount of the miRNA or cDNA reversed transcribed from the miRNA. In some embodiments, the urine for use in the present methods comprises siRNA, and the detecting comprises detecting an amount of the siRNA or cDNA reverse transcribed from the siRNA. In some embodiments, the urine for use in the present methods comprises circular RNA, and the detecting comprises detecting an amount of the circular RNA or cDNA reversed transcribed from the circular RNA. In some embodiments, the urine for use in the present methods comprises long non-coding RNA, and the detecting comprises detecting an amount of the long non-coding RNA or cDNA reversed transcribed from the long non-coding RNA. In some embodiments, the urine for use in the present methods comprises piRNA, and the detecting comprises detecting an amount of the piRNA or cDNA reversed transcribed from the piRNA. In some embodiments, the urine for use in the present methods comprises rRNA, and the detecting comprises detecting an amount of the rRNA or cDNA reversed transcribed from the rRNA. In some embodiments, the urine for use in the present methods comprises tRNA, and the detecting comprises detecting an amount of the tRNA or cDNA reversed transcribed from the tRNA. In some embodiments, the urine for use in the present methods comprises hnRNA, and the detecting comprises detecting an amount of the hnRNA or cDNA reversed transcribed from the hnRNA. In some embodiments, the urine for use in the present methods comprises ncRNA, and the detecting comprises detecting an amount of the ncRNA or cDNA reverse transcribed from the ncRNA. In some embodiments, the urine is whole urine.

Further provided herein are kits for use in the present methods. In some embodiments, the kits comprise a container for containing the urine sample, and instructions for providing the urine sample. In some embodiments, the container is a sealable container, e.g., with a stoppered vial. In some embodiments, the container is commercially available from DNA Genotek Inc. under the trademark Colli-Pee.

In some embodiments, the container contains a buffer, e.g., for processing of the sample. In some embodiments, the buffer is a urine stabilization buffer. Buffers suitable for sample stabilization are known to those of skill in the art and can be determined based on the type of sample being collected. In some embodiments, the buffer further comprises a preservative for adequate stability of the sample. In some embodiments, the buffer to sample ratio is 1:4. In some embodiments, the buffer to sample ratio is 1:4, 1:2, or 3:4. It will be understood that the container will be large enough to accommodate the sample, e.g., the urine sample, and the buffer.

In some embodiments, the container can contain a volume of about 1 mL to about 50 mL of urine sample. In some embodiments, the container can contain a volume of about 1 mL, about 2 mL, about 3 mL, about 4 mL, about 5 mL, about 6 mL, about 7 mL, about 8 mL, about 9 mL, about 10 mL, about 15 mL, about 20 mL, about 25 mL, about 30 mL, about 35 mL, about 40 mL, about 45 mL, or about 50 mL of urine sample.

In some embodiments, the container contains a preservative. In some embodiments, where the container contains a preservative and urine sample, the preservative to urine sample ratio is about 1:1 (v/v), about 1:2 (v/v), about 1:3 (v/v), about 1:4 (v/v), about 1:5 (v/v), about 2:5 (v/v), about 1:6 (v/v), about 1:7 (v/v), about 1:8 (v/v), about 1:9 (v/v), or about 1:10 (v/v). In some embodiments, the preservative to urine sample ratio is about 1:3 (v/v). In some embodiments, the preservative to urine sample ratio is about 2:5 (v/v). In some embodiments, the preservative is Urine Conservation Medium (UCM). In some embodiments, the preservative is Urinary Analyte Stabilizer (UAS).

The urine sample can be stored, e.g., at low temperature, prior to use in the present methods. In some embodiments, the urine sample can be stored for two weeks or more at a temperature of about −80° C. to about −40° C. prior to use in the present methods. In some embodiments, the urine sample can be stored for two weeks or more at a temperature of about −80° C. or about −40° C. prior to use in the present methods. In some embodiments, the urine sample can be stored for fewer than two weeks at a temperature of about 4° C. prior to use in the present methods. In some embodiments, the urine sample can be stored for about one week at a temperature of about 23° C. prior to use in the present methods.

The urine sample for use in the present methods may be an archival sample, having a known and documented medical outcome, or may be a urine sample from a patient whose ultimate medical outcome is not yet known.

The present methods provide a significantly, surprisingly and unexpectedly greater extraction performance, e.g., as measured by cycle relative threshold (Crt), or a significantly, surprisingly and unexpectedly greater amount of extracted RNA, compared to that provided using a standard method for RNA extraction (e.g., the Thermo Fisher method or Norgen method described herein).

### EXAMPLES

The following Examples are provided to demonstrate and further illustrate certain embodiments and aspects of the present disclosure and are not to be construed as limiting the scope thereof.

### Example 1a

RNA was extracted using reagents from the Urine Total RNA Purification Maxi Kit (High Throughput Maxi Slurry Format) (Norgen Biotech, catalog number 29650).

A sample of about 5 mL of whole urine from a urine sample from a male human subject having a prostate and who had a digital rectal examination (DRE) within about 180 minutes before providing the sample was aliquoted into a conical tube. About 0.35 mL of Slurry C3 (Norgen) and 4.65 mL of Lysis Buffer A (Norgen) were added to the urine. Cells within the urine were lysed by vortexing. About 5 mL of absolute ethanol was then added to the lysate and vortexed. The resultant mixture was then centrifuged for 5 min at 2,000×g, and the supernatant was removed and discarded, leaving the pellet. [0277]500 μL Wash Solution A (Norgen) was then added to the pellet and admixed by pipetting or vortexing. A 96-Well Filter Plate (Norgen) was placed on top of a 96-Well Collection Plate (Norgen), and the pellet admixture was pipetted to a well of the 96-Well Filter Plate. The resultant assembly was then centrifuged at 3,146×g (or 3,900 rpm) for 2 minutes, and flowthrough was removed and discarded. [0278]400 μL of Wash Solution (Norgen) was added to the columns of the Filter Plate and centrifuged at 3,146×g (or 3,900 rpm) for 2 minutes, and flowthrough was removed and discarded. The 96-well filter plate was then placed on a new 96-well PCR plate.

For every on-column reaction to be performed, an admixture of 15 μL of DNase I (Norgen) and 100 μL of Enzyme Incubation Buffer was prepared by gently mixing. 115 μL of DNase I (Norgen) and Enzyme Incubation Buffer (Norgen) was added to the Filter Plate column and centrifuged at 3,146×g (or 3,900 rpm) for 2 minutes. The 96-well Filter plate was placed back on top of the 96-Well Collection plate. The flowthrough present in the 96-well PCR plate was then placed back onto the top of the column and incubated at room temperature for 15 minutes.

500 μL of Wash Solution A (Norgen) was added to each used well of the 96-Well Filter Plate and centrifuged at 3,146×g (or 3,900 rpm) for 2 minutes. The flowthrough was removed and discarded.

The wash was repeated.

The bottom of the 96-Well Filter Plate was patted dry, and the 96-Well Filter Plate and the collection plate were reassembled. The assembly was then centrifuged at 3,146×g (or 3,900 rpm) for 15 minutes to completely dry the plate.

100 μL of Elution Solution A (Norgen) was then added to each used well of the plate, and incubated at room temperature for 2 min. The assembly was then centrifuged at 500 rpm for 2 min, then at 3,000×g for 2 min, to achieve extracted RNA.

### Example 1b

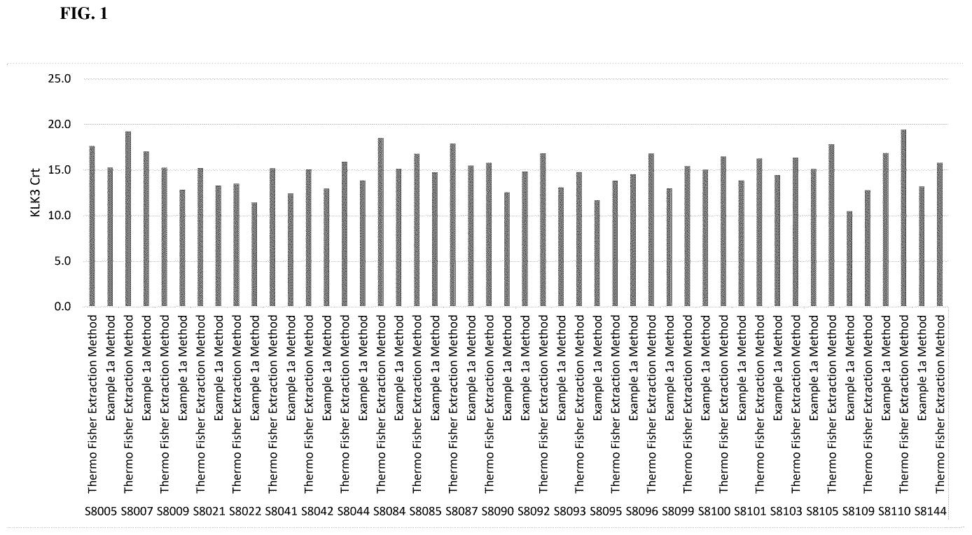

The amount of cDNA reverse transcribed from RNA extracted according to the method of Example 1a was compared to that of an RNA extraction method using the MAGMAX MIRVANA Total RNA Isolation Kit (Thermo Fisher Scientific, Cat No A27828) in accordance with the manufacturer's instructions (referred to herein as the “Thermo Fisher extraction method,” available at https://assets.thermofisher.com/TFS-Assets%2FLSG%2Fmanuals%2FMAN0011139_A27828_magmax_rnaisolation_urine_ug.pdf). Extracted RNA from each extraction method was then processed using the protocol described in Example 2a, to amplify and measure prostate cancer biomarkers. Quantitative polymerase chain reaction (qPCR) of the housekeeping gene KLK3 was measured by relative cycle threshold (Crt) and compared between the two RNA extraction methods in 24 subject samples (FIG. 1). The qPCR was performed in triplicate for each gene target. The amount of cDNA reverse transcribed from RNA extracted according to the method of Example 1a was consistently greater than that reverse transcribed from the cDNA of the Thermo Fisher extraction method, with an average reduction in Crt of 2.2 cycles (Avg Crt: Example 1a=19.9 vs Thermo Fisher extraction method=22.1), which is equivalent to a 4.59 fold increase in RNA extraction efficiency, as shown in FIG. 1.

TABLE 4
Comparison of target gene amplification using the Example
1a method or the Thermo Fisher extraction method

| | | Thermo Fisher | | Fold |
|---|---|---|---|---|
| | Example 1a | Extraction | ΔCrt | Change (x) |
| Gene | Method Cr | Method Crt | (Difference) | (2^{ΔCrt}) |
| APOC1 | 21.4 | 23.0 | 1.6 | 2.90 |
| B3GNT6 | 21.1 | 24.5 | 3.4 | 10.53 |
| CAMKK2 | 17.9 | 20.1 | 2.2 | 4.64 |
| ERG | 26.9 | 28.7 | 1.8 | 3.43 |
| HOXC6 | 23.7 | 25.5 | 1.8 | 3.60 |
| KLK3 | 13.9 | 16.2 | 2.3 | 4.96 |
| KLK4 | 14.3 | 16.0 | 1.8 | 3.44 |
| NKAIN1 | 20.7 | 23.4 | 2.6 | 6.22 |
| OR51E2 | 19.2 | 21.2 | 2.0 | 4.12 |
| PCA3 | 16.8 | 19.0 | 2.2 | 4.55 |
| PCAT14 | 20.8 | 22.6 | 1.8 | 3.48 |
| PCGEM1 | 18.2 | 20.6 | 2.4 | 5.14 |
| SCHLAP1 | 22.0 | 23.9 | 1.9 | 3.84 |
| SPON2 | 19.3 | 21.8 | 2.4 | 5.36 |
| T2ERG | 29.2 | 33.1 | 3.9 | 15.34 |
| TFF3 | 19.3 | 20.9 | 1.7 | 3.19 |
| TMSB15A | 17.9 | 19.6 | 1.6 | 3.12 |
| TRGV9 | 14.8 | 17.1 | 2.3 | 5.10 |
| 18-Gene | 19.9 | 22.1 | 2.2 | 4.59 |
| Average | | | | |

### Example 2a

Reverse transcription reaction mixtures were prepared using Takara PRIMESCRIPT RT reagent PRIMESCRIPT Buffer, random 6mers, and reverse transcriptase (RT) enzyme.

PRIMESCRIPT Buffer, random 6mers, and enzyme were admixed at a ratio of 4:4:1 to generate a master mix. The master mix and extracted RNA (e.g., from Example 1a supra) were admixed at a ratio of 9:11 (for a ratio of RT enzyme to RNA of 1:11). RNA was not normalized. Reaction mixtures were then mixed (e.g., vortexed) and spun down (e.g., by performing a centrifugation).

The following thermal cycling conditions were run: 37° C. for 15 minutes, 85° C. for 5 seconds, and hold at 4° C.

### Example 2b

The reverse transcription efficiency of Example 2a was compared to that of a conventional reverse transcription method (the SUPERSCRIPT IV VILO Master Mix (Thermo Fisher Scientific, Cat No 11756050)), referred to herein as the “Thermo Fisher reverse transcription method”. Reverse transcribed complementary DNA (cDNA) from both methods was processed using the detection protocol of Example 2a to amplify and measure prostate cancer biomarkers. Quantitative polymerase chain reaction (qPCR) of the housekeeping gene KLK3 was measured by relative cycle threshold (Crt) and compared between the two reverse transcription methods across 96 samples. The RNA detection method of Example 2a consistently outperformed the Thermo Fisher reverse transcription method, with an average reduction in Crt of 0.67 cycles (Avg Crt: Example 2a=15.20 vs Thermo Fisher reverse transcription method=15.86), which is equivalent to a 1.59-fold increase in reverse transcription efficiency.

### Example 3

A multi-parameter experiment measured urine input volume, centrifugation time for drying, elution wash volume and final elution volume for RNA extraction from each of three pools (A, B and C) of clinical whole-urine remnants, i.e., whole urine pooled from two or more men having a prostate and who had a DRE within about 180 minutes before providing the urine (Table 2). RNA was extracted as described in Example 1a supra, with the respective parameter modifications as detailed in Table 5: urine volume was 5 mL or 10 mL; drying spin time was 5 min, 10 min or 15 min, and elution 1 and 2 volume was 50 μL or 75 μL.

TABLE 5
Parameters

| | | | | Elution 1 & |
|---|---|---|---|---|
| | | Urine Input | Drying Spin | Elution 2 |
| Sample | Pool | Volume (mL) | Time (min) | Volume (μL) |
| 1 | A | 5 | 5 | 50 |
| 2 | B | 5 | 5 | 50 |
| 3 | C | 5 | 5 | 50 |
| 4 | A | 5 | 5 | 75 |
| 5 | B | 5 | 5 | 75 |
| 6 | C | 5 | 5 | 75 |
| 7 | A | 10 | 5 | 50 |
| 8 | B | 10 | 5 | 50 |
| 9 | C | 10 | 5 | 50 |
| 10 | A | 5 | 10 | 50 |
| 11 | B | 5 | 10 | 50 |
| 12 | C | 5 | 10 | 50 |
| 13 | A | 5 | 10 | 75 |
| 14 | B | 5 | 10 | 75 |
| 15 | C | 5 | 10 | 75 |
| 16 | A | 10 | 10 | 50 |
| 17 | B | 10 | 10 | 50 |
| 18 | C | 10 | 10 | 50 |
| 19 | A | 5 | 15 | 50 |
| 20 | B | 5 | 15 | 50 |
| 21 | C | 5 | 15 | 50 |
| 22 | A | 5 | 15 | 75 |
| 23 | B | 5 | 15 | 75 |
| 24 | C | 5 | 15 | 75 |
| 25 | A | 10 | 15 | 50 |
| 26 | B | 10 | 15 | 50 |
| 27 | C | 10 | 15 | 50 |

RNA extraction performance was determined by measuring the relative cycle threshold (Crt) of cDNA reversed transcribed from extracted RNA expressed by KLK3 by qPCR as described in Example 2a supra. The amplification results are shown in Table 6. An “Undetermined” result designates no detectable amplification. From these results, the combination of parameters that resulted in the lowest KLK3 Crt, the threshold for cDNA reversed transcribed from extracted RNA expressed by KLK3, was a urine input of 5 mL, a drying spin time of 15 min and an Elution 1 and Elution 2 volume of 75 μL.

TABLE 6
Results

| | | Urine Input | | Elution 1 & | | |
|---|---|---|---|---|---|---|
| | | Volume | Drying Spin | Elution 2 | Elution 1 | Elution 2 |
| Sample | Pool | (mL) | Time (min) | Volume (μL) | KLK3 Crt | KLK3 Crt |
| 1 | A | 5 | 5 | 50 | Undetermined | 26.47 |
| 2 | B | 5 | 5 | 50 | Undetermined | 31.23 |
| 3 | C | 5 | 5 | 50 | Undetermined | 28.33 |
| 4 | A | 5 | 5 | 75 | Undetermined | 25.54 |
| 5 | B | 5 | 5 | 75 | 32.84 | 30.08 |
| 6 | C | 5 | 5 | 75 | Undetermined | 29.26 |
| 7 | A | 10 | 5 | 50 | Undetermined | 26.37 |
| 8 | B | 10 | 5 | 50 | Undetermined | 31.86 |
| 9 | C | 10 | 5 | 50 | Undetermined | 27.00 |
| 10 | A | 5 | 10 | 50 | Undetermined | 25.18 |
| 11 | B | 5 | 10 | 50 | Undetermined | 28.40 |
| 12 | C | 5 | 10 | 50 | Undetermined | 27.48 |
| 13 | A | 5 | 10 | 75 | 27.68 | 26.34 |
| 14 | B | 5 | 10 | 75 | 32.61 | 30.11 |
| 15 | C | 5 | 10 | 75 | 27.01 | 28.95 |
| 16 | A | 10 | 10 | 50 | Undetermined | 24.58 |
| 17 | B | 10 | 10 | 50 | Undetermined | 29.25 |
| 18 | C | 10 | 10 | 50 | Undetermined | 26.65 |
| 19 | A | 5 | 15 | 50 | Undetermined | 24.80 |
| 20 | B | 5 | 15 | 50 | Undetermined | 28.11 |
| 21 | C | 5 | 15 | 50 | Undetermined | 27.58 |
| 22 | A | 5 | 15 | 75 | 24.50 | 25.58 |
| 23 | B | 5 | 15 | 75 | 27.52 | 29.08 |
| 24 | C | 5 | 15 | 75 | 26.54 | 29.37 |
| 25 | A | 10 | 15 | 50 | Undetermined | 24.56 |
| 26 | B | 10 | 15 | 50 | 36.14 | 28.84 |
| 27 | C | 10 | 15 | 50 | Undetermined | 26.67 |

### Example 4a

An RNA extraction procedure was performed according to Steps 1 to 25 described below. RNA extraction reagents for this procedure were obtained from the Urine Total RNA Purification Maxi Kit (Norgen Biotek Corp., High Throughput Maxi Slurry Format, Catalogue No. 29650) and the RNase-free DNase I Kit (Norgen Biotek Corp., Catalogue No. 25720).
Step 1. 5 mL of whole urine was aliquoted into a 50 mL conical tube. The whole urine was from a urine sample from a male human subject having a prostate who either had or did not have a digital rectal examination (DRE) within about 180 minutes before providing the sample.
Step 2. 0.35 mL Slurry C3 and 4.65 mL of Lysis Buffer A was added directly to the whole urine. Cells were lysed by vortexing for 15 seconds.
Step 3. 5 mL of absolute ethanol was added to the lysate product of Step 2 and mixed by vortexing for 10 seconds.
Step 4. The admixture was centrifuged for 5 min at 2,000×g and 23° C. The supernatant was discarded following centrifugation.
Step 5. 500 μL of Wash Solution A was added to the pellet and mixed by pipetting or vortexing.
Step 6. A 96-Well Filter Plate was placed on top of a provided 96-Well Collection Plate.
Step 7. The mixture from Step 5 was transferred into a well of the 96-Well Filter Plate.
Step 8. The assembly was centrifuged at 3,146×g (or 3,900 rpm) for 2 minutes at 23° C. The flowthrough was discarded following centrifugation. The 96-Well Filter Plate and the 96-Well Collection Plate were reassembled.
Step 9. 400 μL of Wash Solution (provided with Total RNA Purification Maxi Kit) was added to the well of the 96-Well Filter Plate and centrifuged at 3,146×g (or 3,900 rpm) for 2 minutes at 23° C. The flowthrough was discarded following centrifugation. The 96-Well Filter Plate was placed on a 96-well PCR plate (but the 96-Well Collection Plate was retained).
Step 10. A mix of 15 μL of DNase I and 100 μL of Enzyme Incubation Buffer was prepared in a tube. The tube was gently mixed by inverting a few times. The tube was not vortexed.
Step 11. 115 μL of DNase I/Enzyme Incubation Buffer admixture was added to the well of the 96-Well Filter Plate and centrifuged at 3,146×g (or 3,900 rpm) for 2 minutes at 23° C.
Step 12. The 96-well Filter Plate was placed on top of the 96-Well Collection Plate retained from Step 9.
Step 13. The flowthrough that was present in the well of the 96-well PCR plate was pipetted back into the well of the 96-Well Filter Plate.
Step 14. The 96-Well Filter Plate was incubated at room temperature for 15 minutes.
Step 15. 500 μL of Wash Solution A was added to the well of the 96-Well Filter Plate. The 96-Well Filter Plate and 96-Well Collection Plate assembly were centrifuged at 3,146×g (or 3,900 rpm) for 2 minutes at 23° C.
Step 16. The 96-Well Filter Plate and 96-Well Collection Plate were disassembled and the flowthrough was discarded. The 96-Well Filter Plate and 96-Well Collection Plate were reassembled.
Step 17. Steps 15 and 16 were repeated once.
Step 18. The bottom of the 96-Well Filter Plate was patted dry with a paper towel. The 96-Well Filter Plate and the 96-Well Collection Plate were reassembled.
Step 19. The 96-Well Filter Plate and the 96-Well Collection Plate were centrifuged at 3,146×g (or 3,900 rpm) for 15 minutes at 23° C. to completely dry the plate.
Step 20. 100 μL of Elution Solution A was added to each well of the 96-Well Filter Plate by pipetting the Elution Solution A down the side of the well slowly to soak the entire well.
Step 21. The 96-Well Filter Plate and the 96-Well Collection Plate assembly were incubated on the benchtop at room temperature for 2 minutes.
Step 23. The assembly was centrifuged at 500 RPM for 2 minutes at 23° C.
Step 24. The assembly was centrifuged at 3,146×g (or 3,900 rpm) for 2 minutes at 23° C.
Step 25. The flowthrough was collected and placed in a tube to provide the extracted RNA. The tube was kept on ice or frozen at −80° C. until further analysis.

### Example 4b

A cDNA synthesis procedure was performed using the extracted RNA obtained according to Example 4a. The reverse transcription reagent kit facilitated cDNA synthesis of the extracted RNA prior to analysis by qPCR. Reagents from a PRIMESCRIPT RT Reagent Kit (Perfect Real Time, Takara Bio Inc., Catalogue No. RR037B) were obtained. The Kit included 5× PRIMESCRIPT buffer, Random 6mers, and PRIMESCRIPT RT Enzyme Mix I referenced below. The cDNA synthesis procedure was performed according to Steps 1 to 9 described below.
Step 1. The PRIMESCRIPT RT Reagent Kit was removed from the −20° C. freezer. The 5× PRIMESCRIPT Buffer and Random 6mers were thawed at room temperature. The PRIMESCRIPT RT Enzyme Mix I was kept on ice at about 0° C.
Step 2. Once the reagents were thawed, the 5× PRIMESCRIPT Buffer and Random 6mers were vorexed for 5 seconds.
Step 3. The PRIMESCRIPT RT Enzyme Mix I was mixed by pipetting. The PRIMESCRIPT RT Enzyme Mix I was not vortexed.
Step 4. The Mastermix was prepared by adding 4 μL of 5× PRIMESCRIPT Buffer, 4 μL of Random 6mers and 1 μL of PRIMESCRIPT RT Enzyme Mix I per sample to a 2 mL tube.
Step 5. 9 μL of the Mastermix was added to a well of a 96-well PCR plate.
Step 6. 11 μL of extracted RNA was added to the well of the 96-well PCR plate for a total well volume of 20 μL.
Step 7. The 96-well PCR Plate was sealed with an aluminum seal and vortexed for 10 seconds, then centrifuged for 1 minute at 3,000 rpm.
Step 8. The 96-well PCR plate was incubated using a thermocycler under the conditions for the steps provided in Table 7 below.

TABLE 7

| | | |
|---|---|---|
| Step | Temperature | Time |

| | | | | |
|---|---|---|---|---|
| 1 | 37° | C. | 15 | min |
| 2 | 85° | C. | 5 | Seconds |

| | | | |
|---|---|---|---|
| 3 | 4° | C. | ∞ |

Step 9. When the thermocycler reached 4° C., the 96-well PCR plate was removed from the thermocycler and stored at −20° C. until RT-PCR analysis.

### Example 4c

Twenty-four whole-urine samples were tested: (a) twelve of the twenty-four urine samples were collected from male human subjects who have a prostate and who did not have a DRE on the same day as and before providing the urine sample (non-same-day-DRE urine); and (b) the other 12 urine samples were collected from male human subjects who have a prostate and who had a DRE within about 180 minutes before providing the urine sample (post-DRE urine). RNA was extracted from the non-same-day-DRE urine and post-DRE urine according to Example 4a or the method described in the Norgen Urine Total RNA Purification Maxi 96-Well Kit (Slurry Format) User Manual (“Norgen method”, available at: https://norgenbiotek.com/product/urine-total-rna-purification-maxi-kit-slurry-format?srsltid=AfmBOopVZAEBwz9GmMeOnBOKJD3I1xvWRa_zpFYwfE988aDJrkHZ4Mu3).

The RNA extracted from non-same-day-DRE and post-DRE urine was reverse transcribed to cDNA according to the method of Example 4b. The average Crt value for the 18 genes of Table 4 was evaluated for the cDNA reverse transcribed from the RNA extracted from the non-same-day-DRE and post-DRE urine samples.

Using non-same-day-DRE urine, detection of cDNA reverse transcribed from RNA extracted according to the method of Example 4a (94% 18-gene average cDNA detection) was significantly greater than detection of cDNA reverse transcribed from RNA extracted according to the Norgen method (77% 18-gene average cDNA detection) (Table 8). Also using non-same-day-DRE urine, detection of cDNA reverse transcribed from RNA extracted according to the method of Example 4a required significantly fewer Crt cycles (indicating stronger cDNA signals) than for cDNA reverse transcribed from RNA extracted according to the Norgen method (Table 9). Overall, the cDNA reverse transcribed from the RNA extracted from non-same-day-DRE urine according to the method of Example 4a had a 19.59× increase compared to the cDNA reverse transcribed from the RNA extracted according to the Norgen method (Table 9).

TABLE 8
Improvement of detection of cDNA reverse transcribed from
mRNA extracted from non-same-day-DRE urine of Example 4a

| | | | Percent |
|---|---|---|---|
| | Method of | Norgen | Improvement |
| Gene | Example 4a | Method | (Difference) |
| APOC1 | 100% | 92% | 8% |
| B3GNT6 | 100% | 100% | 0% |
| CAMKK2 | 100% | 83% | 17% |
| ERG | 67% | 17% | 50% |
| HOXC6 | 100% | 42% | 58% |
| KLK3 | 100% | 100% | 0% |
| KLK4 | 100% | 100% | 0% |
| NKAIN1 | 92% | 50% | 42% |
| OR51E2 | 100% | 83% | 17% |
| PCA3 | 100% | 100% | 0% |
| PCAT14 | 100% | 92% | 8% |
| PCGEM1 | 100% | 92% | 8% |
| SCHLAP1 | 100% | 67% | 33% |
| SPON2 | 100% | 83% | 17% |
| TFF3 | 100% | 83% | 17% |
| T2ERG | 42% | 0% | 42% |
| TMSB15A | 100% | 100% | 0% |
| TRGV9 | 100% | 100% | 0% |
| 18-Gene Average | 95% | 77% | 18% |

TABLE 9
Detection of cDNA reverse-transcribed from mRNA extracted
from non-same-day-DRE urine of Example 4a

| | Method of | Norgen | | |
|---|---|---|---|---|
| | Example | Method | | Fold Change |
| Gene | 4a Crt | Crt | ΔCrt | (x) (2^{ΔCrt}) |
| APOC1 | 23.67 | 27.67 | 4.00 | 16.02 |
| B3GNT6 | 22.21 | 23.06 | 0.85 | 1.80 |
| CAMKK2 | 19.96 | 24.76 | 4.80 | 27.92 |
| ERG | 29.37 | 33.61 | 4.24 | 18.87 |
| HOXC6 | 25.51 | 32.24 | 6.73 | 106.37 |
| KLK3 | 16.11 | 20.25 | 4.15 | 17.74 |
| KLK4 | 15.90 | 20.11 | 4.22 | 18.60 |
| NKAIN1 | 23.40 | 29.50 | 6.10 | 68.48 |
| OR51E2 | 22.13 | 25.99 | 3.86 | 14.47 |
| PCA3 | 20.22 | 22.95 | 2.73 | 6.63 |
| PCAT14 | 23.28 | 26.78 | 3.50 | 11.31 |
| PCGEM1 | 19.91 | 24.05 | 4.14 | 17.58 |
| SCHLAP1 | 23.40 | 29.04 | 5.64 | 49.87 |
| SPON2 | 21.08 | 27.79 | 6.71 | 104.58 |
| TFF3 | 21.49 | 26.88 | 5.39 | 41.96 |
| T2ERG | 31.47 | 35.00 | 3.53 | 11.59 |
| TMSB15A | 20.40 | 23.32 | 2.93 | 7.60 |
| TRGV9 | 16.60 | 20.35 | 3.75 | 13.42 |
| 18-Gene Average | 22.01 | 26.30 | 4.29 | 19.59 |

Using post-DRE urine, detection of cDNA reverse transcribed from RNA extracted according to the method of Example 4a (96% 18-gene average cDNA detection) was greater than detection of cDNA reverse transcribed from RNA extracted according to the Norgen method (95% 18-gene average cDNA detection) (Table 10). Moreover, there was a significant improvement in detection of cDNA reversed transcribed from RNA expressed by ERG and T2ERG using the RNA extraction method of Example 4a compared to the Norgen method (Table 10). Also using post-DRE urine, detection of cDNA reverse transcribed from RNA extracted according to the method of Example 4a required fewer Crt cycles (indicating stronger cDNA signals) than for cDNA reverse transcribed from RNA extracted according to the Norgen method (Table 11). Overall, the cDNA reverse transcribed from the RNA extracted from post-DRE urine according to the method of Example 4a had an 8.77× increase compared to the cDNA reverse transcribed from the RNA extracted according to the Norgen method (Table 11).

TABLE 10
Improvement of detection of cDNA reverse transcribed
from RNA extracted from post-DRE urine of Example 4a

| | | | Percent |
|---|---|---|---|
| | Method of | Norgen | Improvement |
| Gene | Example 4a | Method | (Difference) |
| APOC1 | 100% | 100% | 0% |
| B3GNT6 | 100% | 100% | 0% |
| CAMKK2 | 100% | 100% | 0% |
| ERG | 83% | 67% | 16% |
| HOXC6 | 100% | 100% | 0% |
| KLK3 | 100% | 100% | 0% |
| KLK4 | 100% | 100% | 0% |
| NKAIN1 | 100% | 100% | 0% |
| OR51E2 | 100% | 100% | 0% |
| PCA3 | 100% | 100% | 0% |
| PCAT14 | 100% | 100% | 0% |
| PCGEM1 | 100% | 100% | 0% |
| SCHLAP1 | 100% | 100% | 0% |
| SPON2 | 100% | 100% | 0% |
| TFF3 | 100% | 100% | 0% |
| T2ERG | 50% | 42% | 8% |
| TMSB15A | 100% | 100% | 0% |
| TRGV9 | 100% | 100% | 0% |
| 18-Gene Average | 96% | 95% | 1% |

TABLE 11
Detection of cDNA reverse-transcribed from RNA
extracted from post-DRE urine of Example 4a

| | Method of | Norgen | | Fold |
|---|---|---|---|---|
| | Example 4a | Method | ΔCrt | Change (x) |
| Gene | Crt | Crt | (Difference) | (2^{ΔCrt}) |
| APOC1 | 20.63 | 23.74 | 3.11 | 8.61 |
| B3GNT6 | 20.72 | 22.33 | 1.61 | 3.05 |
| CAMKK2 | 16.74 | 20.06 | 3.32 | 9.99 |
| ERG | 25.92 | 29.65 | 3.73 | 13.26 |
| HOXC6 | 23.54 | 25.51 | 1.97 | 3.92 |
| KLK3 | 12.92 | 16.20 | 3.28 | 9.71 |
| KLK4 | 12.90 | 16.01 | 3.11 | 8.60 |
| NKAIN1 | 19.99 | 22.62 | 2.63 | 6.18 |
| OR51E2 | 18.20 | 22.22 | 4.02 | 16.21 |
| PCA3 | 16.76 | 20.33 | 3.56 | 11.83 |
| PCAT14 | 20.03 | 23.42 | 3.40 | 10.53 |
| PCGEM1 | 16.72 | 20.07 | 3.35 | 10.19 |
| SCHLAP1 | 21.30 | 23.49 | 2.19 | 4.56 |
| SPON2 | 18.47 | 21.22 | 2.75 | 6.75 |
| TFF3 | 17.89 | 21.61 | 3.73 | 13.24 |
| T2ERG | 29.81 | 31.58 | 1.77 | 3.42 |
| TMSB15A | 17.12 | 20.48 | 3.36 | 10.28 |
| TRGV9 | 13.80 | 16.71 | 2.91 | 7.50 |
| 18-Gene Average | 19.08 | 22.07 | 2.99 | 8.77 |

1. A method for extracting RNA present in whole urine from a human subject's urine sample, comprising:
admixing the whole urine and a first solid support to provide an RNA-bound first solid support, wherein the first solid support comprises silicon carbide;
admixing the RNA-bound first solid support and a polar organic solvent to provide an RNA-bound first solid support admixture and performing centrifugation of the RNA-bound first solid support admixture to provide a pellet and a supernatant;
removing the supernatant, and washing the pellet with a wash buffer to provide a washed RNA-bound first solid support;
admixing wash buffer, the washed RNA-bound first solid support, and a second solid support to provide an RNA-bound second solid support, wherein the second solid support comprises a silicon-based compound;
washing the RNA-bound second solid support to provide a washed RNA-bound second solid support;
treating the washed RNA-bound second solid support with deoxyribonuclease (DNase) to provide a DNase-treated second solid support and a flowthrough;
washing the DNase-treated second solid support to provide a washed DNase-treated second solid support;
drying the washed DNase-treated second solid support to provide a dried second solid support; and
eluting RNA from the dried second solid support to provide extracted RNA.

2. The method of claim 1, wherein the whole urine comprises first-catch urine.

3. The method of claim 1, wherein the whole urine has a volume of about 5 mL to about 10 mL.

4. (canceled)

5. The method of claim 1, wherein the method does not comprise isolating an exosome from the whole urine.

6. The method of claim 1, wherein the first solid support is one of a plurality of silicon carbide particles, and the method comprises admixing the whole urine and the plurality of silicon carbide particles.

7. The method of claim 6, wherein the plurality of silicon carbide particles are present in a slurry, and the method comprises admixing the whole urine and the slurry.

8. (canceled)

9. The method of claim 7, wherein the admixing the whole urine and the slurry occurs in the presence of a lysis buffer.

10-13. (canceled)

14. The method of claim 1, wherein the polar organic solvent is ethanol.

15-16. (canceled)

17. The method of claim 1, wherein the admixing the wash buffer, the washed RNA-bound first solid support, and the second solid support comprises performing a centrifugation of the wash buffer, washed RNA-bound first solid support, and the second solid support to provide the RNA-bound second solid support.

18-25. (canceled)

26. The method of claim 1, wherein the washing the RNA-bound second solid support comprises admixing the RNA-bound second solid support and a wash buffer to provide an RNA-bound second solid support and wash buffer admixture, and performing a centrifugation of the RNA-bound second solid support and wash buffer admixture to provide the washed RNA-bound second solid support.

27-29. (canceled)

30. The method of claim 1, wherein the treating comprises performing a centrifugation of the washed RNA-bound second solid support and the deoxyribonuclease (DNase) to provide the DNase-treated second solid support and the flowthrough.

31-33. (canceled)

34. The method of claim 30, wherein the flowthrough is a first flowthrough, and wherein the treating further comprises admixing the DNase-treated second solid support and the first flowthrough to provide a DNase-treated second solid support admixture and incubating the DNase-treated second solid support admixture at room temperature for about 15 minutes to provide an incubated DNase-treated solid support admixture.

35. The method of claim 34, further comprising admixing the incubated DNase-treated second solid support admixture and a wash buffer to provide a DNase-treated second solid support and wash buffer admixture and performing a centrifugation of the DNase-treated second solid support and wash buffer admixture to provide the washed DNase-treated second solid support and a second flowthrough.

36-43. (canceled)

44. The method of claim 1, wherein the eluting comprises admixing the dried second solid support and an elution buffer to provide an elution support admixture, incubating the elution support admixture to provide an incubated elution support admixture, and performing a centrifugation of the incubated elution support admixture to provide the extracted RNA.

45-49. (canceled)

50. The method of claim 44, wherein the centrifugation of the incubated elution support admixture is a first centrifugation of the incubated elution support admixture, and the method further comprises performing a second centrifugation of the incubated elution support admixture.

51-52. (canceled)

53. The method of claim 1, further comprising detecting the extracted RNA.

54. The method of claim 53, wherein the detecting comprises reverse transcribing the extracted RNA to complementary DNA (cDNA) using a reverse transcriptase and detecting the cDNA.

55-58. (canceled)

59. The method of claim 1, wherein the human subject is a male human subject who has a prostate and is prostate-biopsy naïve or prostate biopsy-prior negative.

60. (canceled)

61. The method of claim 1, wherein the RNA is expressed by a gene of the subject.

62-63. (canceled)

64. The method of claim 61, wherein the gene is a prostate cancer gene.

65. (canceled)

66. The method of claim 64, wherein the prostate cancer gene is ACSM1, AMACR, AR, COL9A2, CRISP3, CST2, DLX1, ETV1, F5, GDF15, GLYATL1, GOLM1, GRIN3A, LINC00993, LRRN1, MIPEP, MS4A8, MYO6, PCA3.1, PDLIM5, PEX10, PLA1A, PLA2G7, PRCAT47, SPINK1, TDO2, TK1, TMEFF2, TRGV9.1, VSTM2L, TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, HOXC6, or KLK3.

67. (canceled)

68. The method of claim 64, wherein the prostate cancer gene is TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, HOXC6, or KLK3.

69-73. (canceled)

74. The method of claim 1, wherein the urine sample is provided outside a clinical setting.

75-77. (canceled)

78. The method of claim 1, wherein the human subject has or is suspected of having prostate cancer.

79. The method of claim 78, wherein the prostate cancer is Grade Group (GG)≥2 prostate cancer.

80. The method of claim 78, wherein the prostate cancer is GG≥3 prostate cancer.

81. The method of claim 78, wherein the prostate cancer is GG≥4 prostate cancer.

82. The method of claim 78, wherein the prostate cancer is GG5 prostate cancer.

83-87. (canceled)

88. The method of claim 1, wherein the method does not comprise admixing the whole urine and the first solid support under reduced pressure.

89-91. (canceled)

92. A composition comprising whole urine and a solid support comprising silicon carbide, wherein the whole urine is from a urine sample of a male human subject who has a prostate and who did not have a digital rectal examination (DRE) within about 180 minutes before providing the urine sample.

93-103. (canceled)

104. A method for making a plurality of RNA-bound solid supports, comprising admixing whole urine and a plurality of solid supports comprising silicon carbide to provide the plurality of RNA-bound solid supports, wherein the whole urine is from a urine sample of a male human subject who has a prostate and who did not have a digital rectal examination (DRE) within about 180 minutes before providing the urine sample.

105. (canceled)

106. The method of claim 104, wherein the method does not comprise admixing whole urine and the plurality of solid supports under reduced pressure.

107. (canceled)

108. The method of claim 1, wherein the human subject did not have a digital rectal examination (DRE) within about 180 minutes before providing the urine sample.


SEQUENCE LISTING (147 sequences)

SEQ ID NO: 1 - DNA, 293 bp, Homo sapiens
  source 1..293: mol_type = genomic DNA
taggcgcgagctaagcaggaggcggaggcggaggcggagggcgaggggcggggagcgccgcctggagcgcggcaggaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaag

SEQ ID NO: 2 - DNA, 1574 bp, Homo sapiens
  source 1..1574: mol_type = genomic DNA
gcttttatgagctgtaacactcaccgcgaaggtccgcagcttcactcctgaagccagcgagaccacgagcctactgggaggaacgaacaactcccgacgcgccgccttaagagctgtaacactcaccgcgaaggtctgcagcttcactcctgagccagcgagaccacgaacccaccagaaggaaaaaactccgaacacatctgaacatcagaagcaacaaactccggacacgccgcctttaagaactgtaacactcactgcgagggtccgcggcttcattcttgaagtgagtgagaccaagaacccaccagttctggacacaatttcaagtcctcaggtgccatcaatattctgaaaatggcagtgatttttattcaacctgtataaggcactttcaccatgtacctggaagcaacatctacatctttttcagtttcttctacgccaggtgtgtgcttagctccatgacaaaaggtgacagcttattctgcagcacacacacatcatcaaagtgggaggtggtgagactggcacactgacagtctgtcctagcagatttcagctcacactgcaatctagatgctggggacacaaggtccaccttccaggaatatggccatgacaccagaaatcacaaacatgatgagaatggaatgactggggaagaagtgccagatgcttcacttgtaaatgaagacccagcctctggggatgcagataccacctccctgaagaagctgaatatctgcagataagtggagttcaccaatgatgaggagcgggatggagaaaggaggtagggagagtcatccaaggaacatgagcaacatgttaaaagccaagtggtttaatttctggagatggtgaacccaagaggctctgctgggagacaacaaaaataatgaagaattgaaccagagtccggtgaatatcagcactgggaccagttagcagaggaaaaggaaagaataaaagcgaaaagaatgaagagtcatatgattaccaacttttcctttttcatataaattgagtgtatatgggtctggaacaacctgaatttccatcaagtcctggctaacctcattatgtcctatgaatatttttgactaatcccactttacattaatctgtattgtgaatgtggatattgaattatatttctttgtaatcccattatccaaaatccagttcagagactattagttaccaatgttcactgtgaaggaaaaaaaaaaaaaaaaagctcagaggataaacatgtgatatggtttggctgtgtccccacccaaatatcatcttgaattgtagctcccataattcccacgtgttgtgggagggacccggtgggagataattgtatcatgggggtggttcccccatactattctcatagtagtgaataagtctcacaaaatctgatggttttatgagggaaaacccctttcacctggttctcattctcttctctggtctgtcgtcatgtaagacatgcctttcaccttctccaccatgactgtgaggcctccccagccacgtggaactgtgagcccattaaacctctttcacttataaat

SEQ ID NO: 3 - DNA, 2792 bp, Homo sapiens
  source 1..2792: mol_type = genomic DNA
cttctgggaatctccacaccctgaagacacagtgagttagcaccaccaccaggaattggcctttcagctctgtgcctgtctccagtcaggctggaataagtctcctcatatttgcaagctcggccctcccctggaatctaaagcctcctcagccttctgagtcagcctgaaaggaacaggccgaactgctgtatgggctctactgccagtgtgacctcaccctctccagtcacccctcctcagttccagctatgagttcctgcaacttcacacatgccacctttgtgcttattggtatcccaggattagagaaagcccatttctgggttggcttccccctcctttccatgtatgtagtggcaatgtttggaaactgcatcgtggtcttcatcgtaaggacggaacgcagcctgcacgctccgatgtacctctttctctgcatgcttgcagccattgacctggccttatccacatccaccatgcctaagatccttgcccttttctggtttgattcccgagagattagctttgaggcctgtcttacccagatgttctttattcatgccctctcagccattgaatccaccatcctgctggccatggcctttgaccgttatgtggccatctgccacccactgcgccatgctgcagtgctcaacaatacagtaacagcccagattggcatcgtggctgtggtccgcggatccctcttttttttcccactgcctctgctgatcaagcggctggccttctgccactccaatgtcctctcgcactcctattgtgtccaccaggatgtaatgaagttggcctatgcagacactttgcccaatgtggtatatggtcttactgccattctgctggtcatgggcgtggacgtaatgttcatctccttgtcctattttctgataatacgaacggttctgcaactgccttccaagtcagagcgggccaaggcctttggaacctgtgtgtcacacattggtgtggtactcgccttctatgtgccacttattggcctctcagtggtacaccgctttggaaacagccttcatcccattgtgcgtgttgtcatgggtgacatctacctgctgctgcctcctgtcatcaatcccatcatctatggtgccaaaaccaaacagatcagaacacgggtgctggctatgttcaagatcagctgtgacaaggacttgcaggctgtgggaggcaagtgacccttaacactacacttctccttatctttattggcttgataaacataattatttctaacactagcttatttccagttgcccataagcacatcagtacttttctctggctggaatagtaaactaaagtatggtacatctacctaaaggactattatgtggaataatacatactaatgaagtattacatgatttaaagactacaataaaaccaaacatgcttataacattaagaaaaacaataaagatacatgattgaaaccaagttgaaaaatagcatatgccttggaggaaatgtgctcaaattactaatgatttagtgttgtccctactttctctctcttttttctttcttttttttttattatggttagctgtcacatacaactttttttttttttgagatggggtctcgctctgtcaccaggctggagtgcagtggcgcgatctcggctcactgcaacctccacatcccatgttgaagtaattcttctgcctcagcctcccgagtagctgggactagaggaacgtgccaccatgactggctaattttctgtattttttagtagagacagagtttcaccatgttggccaggatggtctcgatctcctgaccttgtgatccacccgcctcagcctcccaaagtgttgggattacaggtgtgaaccactgtgcccggcctgtgtacaactttttaaatagggaatatgatagcttcgcatggtggtgtgcacctatagcccccactgcctggaaagctgaggtgggagaatcgcttgagtccaggagtttgaggttacagtgatccacgatcgtaccactacactccagcctgggcaacagagcaagaccctgtctcaaagcataaaatggaataacatatcaaatgaaacagggaaaatgaagctgacaatttatggaagccagggcttgtcacagtctctactgttattatgcattacctgggaatttatataagcccttaataataatgccaatgaacatctcatgtgtgctcacaatgttctggcactattataagtgcttcacaggttttatgtgttcttcgtaactttatggagtaggtaccatttgtgtctctttattataagtgagagaaatgaagtttatattatcaaggggactaaagtcacacggcttgtgggcactgtgccaagatttaaaattaaatttgatggttgaatacagttacttaatgaccatgttatattgcttcctgtgtaacatctgccatttatttcctcagctgtacaaatcctctgttttctctctgttacacactaacatcaatggctttgtacttgtgatgagagataaccttgccctagttgtgggcaacacatgcagaataatcctgttttacagctgcctttcgtgatcttattgcttgcttttttccagattcagggagaatgttgttgtctatttgtctcttacatctccttgatcatgtcttcattttttaatgtgctctgtacctgtcaaaaattttgaatgtacaccacatgctattgtctgaacttgagtataagataaaataaaattttattttaaatttt

SEQ ID NO: 4 - DNA, 514 bp, Homo sapiens
  source 1..514: mol_type = genomic DNA
aggcggtcaggggaaggctcaggaggagggagatcaacatcaacctgccccgccccctccccagcctgataaaggtcctgcgggcaggacaggacctcccaaccaagccctccagcaaggattcagagtgcccctccggcctcgccatgaggctcttcctgtcgctcccggtcctggtggtggttctgtcgatcgtcttggaaggcccagccccagcccaggggaccccagacgtctccagtgccttggataagctgaaggagtttggaaacacactggaggacaaggctcgggaactcatcagccgcatcaaacagagtgaactttctgccaagatgcgggagtggttttcagagacatttcagaaagtgaaggagaaactcaagattgactcatgaggacctgaagggtgacatcccaggaggggcctctgaaatttcccacaccccagcgcctgtgctgaggactccctccatgtggccccaggtgccaccaataaaaatcctacagaaaa

SEQ ID NO: 5 - DNA, 1201 bp, Homo sapiens
  source 1..1201: mol_type = genomic DNA
gagatacggcctcgtgggaagggaaagacctgaccgtcccccagcccgacacccgtaaagggtctgtgctgaggaggattagtaaaaggggaaggcctcttgcagttgagataagaggaaggcctccgtctcctgcatgtccttgggaatggaatgtcttggtgtaaaacccgatagtacattccttctattctgagagaagaaaaccaccctgtggctggagggtgaaggtactctacagtgtggtcattgaggacaagttgacgagagagtcccaagtacgtccacggtcagccttgcgacatttaaagttctacaatgaactcactggagatgcaaagaaaagtgtggagatggagacaccccaatcgactcgccagtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaatt

SEQ ID NO: 6 - DNA, 4905 bp, Homo sapiens
  source 1..4905: mol_type = genomic DNA
agagcaagctgagccgagccgagccgagctgggggcgcagagcgcgggaggcggcggcggcgcggagcccaggtggctccgctgccggatgggagtgccccagtgtgctggatgaagctggcgcatgcaccatgtcatcatgtgtctctagccagcccagcagcaaccgggccgccccccaggatgagctggggggcaggggcagcagcagcagcgaaagccagaagccctgtgaggccctgcggggcctctcatccttgagcatccacctgggcatggagtccttcattgtggtcaccgagtgtgagccgggctgtgctgtggacctcggcttggcgcgggaccggcccctggaggccgatggccaagaggtcccccttgacacctccgggtcccaggcccggccccacctctccggtcgcaagctgtctctgcaagagcggtcccagggtgggctggcagccggtggcagcctggacatgaacggacgctgcatctgcccgtccctgccctactcacccgtcagctccccgcagtcctcgcctcggctgccccggcggccgacagtggagtctcaccacgtctccatcacgggtatgcaggactgtgtgcagctgaatcagtataccctgaaggatgaaattggaaagggctcctatggtgtcgtcaagttggcctacaatgaaaatgacaatacctactatgcaatgaaggtgctgtccaaaaagaagctgatccggcaggccggctttccacgtcgccctccaccccgaggcacccggccagctcctggaggctgcatccagcccaggggccccattgagcaggtgtaccaggaaattgccatcctcaagaagctggaccaccccaatgtggtgaagctggtggaggtcctggatgaccccaatgaggaccatctgtacatggtgttcgaactggtcaaccaagggcccgtgatggaagtgcccaccctcaaaccactctctgaagaccaggcccgtttctacttccaggatctgatcaaaggcatcgagtacttacactaccagaagatcatccaccgtgacatcaaaccttccaacctcctggtcggagaagatgggcacatcaagatcgctgactttggtgtgagcaatgaattcaagggcagtgacgcgctcctctccaacaccgtgggcacgcccgccttcatggcacccgagtcgctctctgagacccgcaagatcttctctgggaaggccttggatgtttgggccatgggtgtgacactatactgctttgtctttggccagtgcccattcatggacgagcggatcatgtgtttacacagtaagatcaagagtcaggccctggaatttccagaccagcccgacatagctgaggacttgaaggacctgatcacccgtatgctggacaagaaccccgagtcgaggatcgtggtgccggaaatcaagctgcacccctgggtcacgaggcatggggcggagccgttgccgtcggaggatgagaactgcacgctggtcgaagtgactgaagaggaggtcgagaactcagtcaaacacattcccagcttggcaaccgtgatcctggtgaagaccatgatacgtaaacgctcctttgggaacccattcgagggcagccggcgggaggaacgctcactgtcagcgcctggaaacttgctcaccaaaaaaccaaccagggaatgtgagtccctgtctgagctcaaggaagcaaggcagcgaagacaacctccagggcaccgacccgccccccgtgggggaggaggaagtgctcttgtgagaggcagtccctgcgtggaaagttgctgggcccccgcccccggctcccccgcacgcatgcatccactgcggccggaggaggccatggagcccgagtagctgcctggatcgctcgacctcgcatgcgcgccgcgtcgcctctggggggctgctgcaccgcgtttccatagcagcatgtcctacggaaacccagcacgtgtgtagagcctcgatcgtcatctctggttatttgttttttcctttgttgttttaaaggggacaaaaaaaaaaaaaggacttgactccatgacgtcgaccgtggccgctggctggctggacaggcgggtgtgaggagttgcagacccaaacccacgtgcattttgggacaattgctttttaaaacgtttttatgccaaaaatccttcattgtgattttcagaaccacgtcagatataccaagtgactgtgtgtggggtttgacaactgtggaaaggcgagcagaaaactccggcggtctgaggccatggaggtggttgctgcatttgagagggagtagggggctagatgtggctcctagtgcaaaccggaaaccatggcaccttccagagccgtggtctcaaggagtcagagcagggctggccctcagtagctgcagggagctttgatgcaacttatttgtaagaaggatttttaaattttttatgggtagaattgtagtcaggaaaacagaaagggcttgaaatttaataagtgctgctggaaggggattttccaagcctggaagggtattcagcagctgtggtggggaaacatttctcctgaaagactgaacgtgtttcttcatgacagctgctcaaagcaggtttctgagatagctgaccgagctctggtaaatctctttgtcaaattacgaaaacttcagggtgaaatcctatgcttccatgtacattacatggcttaagattaaacaaaaacatttttcaagtctctaactagagtgaactctagagcacagtagttcagaaactatttagagcttccaggatatatttcacagcttcaggcatgtgatcagttagagccgatgaaacctatgcccgcctgtatatatattagcagcttagctagttcataacctgtatattctaaagactgctaaggttttgttttcattttaaatcctagctgattgttgtggtcaatgaaatacccagtttctggagggccaggtgggaaatgctttcactggaccaacacacaaatgatcatcctgaggatctgagcttccctagactccacacaataaccttggggcacccttttagagaagactgttgaaacccacagcactcgttggggtatgaggaaaccagggcttggcacaggaagttcccctttgtagctaaaagtccagaaagaaagggttcatctttttgacttccaactgatattgggaagtttggttgaggttcaagtgtgactccttccagagccacaggtaggggagtgtgaagttgagggggaggaaagctggaaggactctgccttgggagattcccagctctgctttccagcgcttggtggaatctgggctggggaaagacggcaccgggaaactctgcttccccattgtttccatctgatcagctgtggtgtgaggacttctcagacaaaggcaaggcctcgtgcccctgcccagcccattcatggagccctgggccttcttggcttccatagatcctaagctcttgactgtagtttagccagacttgttttgctatcttataagcagttcagaattagggaatgctggttttgaagagcaaaggacaggtagtctagagagggtcgtctggcctgcttgctgggtctttgtaacccagcacttcctcttgccctcctggctttatgtttatggggagaggactcaatagctccaccccttctggcaccagatggggcttggttagtttgcaataagcaccttgcagaggttaaagccagcgggtccctagtcttaggcccagcctgcttgtgtgggctctggcctggcctggtggctggcccagggggcagcagtgcttagagcttctgcagggcttctcttgtttacacagctgcatcagacaatgccatttctccccaccacggaaccttccatctaagatttcttccagggaatgccagcaatcaggcagcacccagctgtgggggcagtggggtgggggagacccacattgatgactttttttttttcttttaatgaagaaacaccaaagaaagctgtggaaaggacctgccccacatgaaaaggataagccaagatggctgtaaacacagagcatttgagctgccactcttggagcacattgatttttcaaaagccagctctgtcaggaaaggaggtgctgttatgagcagctcttccagtgggcaaagaggacgcccataatttcttccattgctagctcatctgtgggaccaatttggtgtaagcaacctgtggcctgcacttgtggcctcgaaggaagcacaaaccctccatccacttcccatttcctctgcccttttccacctcccccttccatcccaccagctgccagtggctcccagaaagccttattgagccccttgttgacacttggggctgcggaggcctctccctactggtctggcctttcctgagaggcaggtcttccgtcctcagagcctttctggaacaaggagaatgcctgtgcaggtggacacacaggcctggcctgtcgctctcacttgtcttccagcggggagcttcacgttgccgagtggaagaaccatgacctccacttgcttccaaggtgctagggaagtttcagggtacgctggttcccctctccagctggaggccgagtttctggggactgcagatttttctactctgtgatcgattcaatgcccgatgcttctgtttcattcccgaccctttctactatgcattttccttttatcaggtgtataaagttaaatactgtgtatttatcactaaaaagtacatgaacttaagagacaactaagcctttcgtgtttttccacaggtgtttaagcttctctgtacagttgaaataaacagacagcaaaatggtgccaa

SEQ ID NO: 7 - DNA, 3757 bp, Homo sapiens
  source 1..3757: mol_type = genomic DNA
acagaagaaatagcaagtgccgagaagctggcatcagaaaaacagaggggagatttgtgtggctgcagccgagggagaccaggaagatctgcatggtgggaaggacctgatgatacagaggtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagagatccctgggagaaatgcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggattacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcattttgttcaaagacccttcgtgttgctgcctaatatgtagctgactgtttttcctaaggagtgttctggcccaggggatctgtgaacaggctgggaagcatctcaagatctttccagggttatacttactagcacacagcatgatcattacggagtgaattatctaatcaacatcatcctcagtgtctttgcccatactgaaattcatttcccacttttgtgcccattctcaagacctcaaaatgtcattccattaatatcacaggattaacttttttttttaacctggaagaattcaatgttacatgcagctatgggaatttaattacatattttgttttccagtgcaaagatgactaagtcctttatccctcccctttgtttgattttttttccagtataaagttaaaatgcttagccttgtactgaggctgtatacagccacagcctctccccatccctccagccttatctgtcatcaccatcaacccctcccatgcacctaaacaaaatctaacttgtaattccttgaacatgtcaggcatacattattccttctgcctgagaagctcttccttgtctcttaaatctagaatgatgtaaagttttgaataagttgactatcttacttcatgcaaagaagggacacatatgagattcatcatcacatgagacagcaaatactaaaagtgtaatttgattataagagtttagataaatatatgaaatgcaagagccacagagggaatgtttatggggcacgtttgtaagcctgggatgtgaagcaaaggcagggaacctcatagtatcttatataatatacttcatttctctatctctatcacaatatccaacaagcttttcacagaattcatgcagtgcaaatccccaaaggtaacctttatccatttcatggtgagtgcgctttagaattttggcaaatcatactggtcacttatctcaactttgagatgtgtttgtccttgtagttaattgaaagaaatagggcactcttgtgagccactttagggttcactcctggcaataaagaatttacaaagagctactcaggaccagttgttaagagctctgtgtgtgtgtgtgtgtgtgtgagtgtacatgccaaagtgtgcctctctctctttgacccattatttcagacttaaaaacaagcatgttttcaaatggcactatgagctgccaatgatgtatcaccaccatatctcattattctccagtaaatgtgataataatgtcatctgttaacataaaaaaagtttgacttcacaaaagcagctggaaatggacaaccacaatatgcataaatctaactcctaccatcagctacacactgcttgacatatattgttagaagcacctcgcatttgtgggttctcttaagcaaaatacttgcattaggtctcagctggggctgtgcatcaggcggtttgagaaatattcaattctcagcagaagccagaatttgaattccctcatcttttaggaatcatttaccaggtttggagaggattcagacagctcaggtgctttcactaatgtctctgaacttctgtccctctttgtgttcatggatagtccaataaataatgttatctttgaactgatgctcataggagagaatataagaactctgagtgatatcaacattagggattcaaagaaatattagatttaagctcacactggtcaaaaggaaccaagatacaaagaactctgagctgtcatcgtccccatctctgtgagccacaaccaacagcaggacccaacgcatgtctgagatccttaaatcaaggaaaccagtgtcatgagttgaattctcctattatggatgctagcttctggccatctctggctctcctcttgacacatattagcttctagcctttgcttccacgacttttatcttttctccaacacatcgcttaccaatcctctctctgctctgttgctttggacttccccacaagaatttcaacgactctcaagtcttttcttccatccccaccactaacctgaatgcctagacccttatttttattaatttccaatagatgctgcctatgggctatattgctttagatgaacattagatatttaaagctcaagaggttcaaaatccaactcattatcttctctttctttcacctccctgctcctctccctatattactgattgcactgaacagcatggtccccaatgtagccatgcaaatgagaaacccagtggctccttgtggtacatgcatgcaagactgctgaagccagaaggatgactgattacgcctcatgggtggaggggaccactcctgggccttcgtgattgtcaggagcaagacctgagatgctccctgccttcagtgtcctctgcatctcccctttctaatgaagatccatagaatttgctacatttgagaattccaattaggaactcacatgttttatctgccctatcaattttttaaacttgctgaaaattaagttttttcaaaatctgtccttgtaaattactttttcttacagtgtcttggcatactatatcaactttgattctttgttacaacttttcttactcttttatcaccaaagtggcttttattctctttattattattattttcttttactactatattacgttgttattattttgttctctatagtatcaatttatttgatttagtttcaatttatttttattgctgacttttaaaataagtgattcggggggtgggagaacaggggagggagagcattaggacaaatacctaatgcatgtgggacttaaaacctagatgatgggttgataggtgcagcaaaccactatggcacacgtatacctgtgtaacaaacctacacattctgcacatgtatcccagaacgtaaagtaaaatttaaaaaaaagtga

SEQ ID NO: 8 - DNA, 2922 bp, Homo sapiens
  source 1..2922: mol_type = genomic DNA
agtgctgctctgcgctgcgccgcgctcggggctcgctctccttgctccgcgctccccgccagccgccccggggcaggaggcgcgcctgacggacggcccgctagacaaaggaggcgcggctcggcggggccagcgcgcggacggacggaccatggactcggagcgcgggcggccggccccagccttggggaccggacactcccgggcccggccctaggcgcccggccccgccgcccggcgcgcccagcggggaggacgtggagcccgcgcggcgcgagcaggcggcggccgcggagcaagaagggcgccgcggcgtgcggcccgcgcagcccccggagccatgggcaagtgcagcgggcgctgcacgctggtcgccttctgctgcctgcagctggtggctgcgctggagcggcagatctttgacttcctgggctaccagtgggctcccatcctagccaacttcctgcacatcatggcagtcatcctgggcatctttggcaccgtgcagtaccgctcccggtacctcatcctgtatgcagcctggctggtgctctgggttggctggaatgcatttatcatctgcttctacttggaggttggacagctgtcccaggaccgggacttcatcatgaccttcaacacatccctgcaccgctcctggtggatggagaatgggccaggctgcctggtgacacctgttctgaactcccgcctggctctggaggaccaccatgtcatctctgtcactggctgcctgcttgactacccctacattgaagccctcagcagcgccctgcagatcttcctggcactgttcggcttcgtgttcgcctgctacgtgagcaaagtgttcctggaggaggaggacagctttgacttcatcggcggctttgactcctacggataccaggcgccccagaagacgtcgcatttacagctgcagcctctgtacacgtcggggtagcctctgccccgcgcccaccccggcgcctcgccctgggctgaccgcagctgccgcgagctcgggccaaggcgcaggcgtgtccccctggtggcccgcgcgctcactgcagcctgtgcccaaccccgcgtctgcatctggagatgcggacttggacgtggacttggacttggacttggatttgagcttggctcttcgcagcccggacttcggaggagtggggcggggcgggggaggggcaccacgggttttttgttttttgtttgtttgtttttaatctcagccttggcgtgagctggggccttcctctcttctccagcctctccctttcactcttcacccagcatcctgcccccctgtccaaaaacagcaggacatcagacccatcccatcccaccacactcactcaccagctctggggaaagctactgtgaactaggagcaggattcctgggttctaatcgcaggtccatcactgactgtgacgtctagcaaagcccttgccctctctgagcctcggtttccgcacctcaagtaattaatcccttagcaaatggactcttttagacttctcatttaactcaattccctgagctagactgggattaaaattctcattttgcagtacattaaaactgaggcccagagatgtgatttgcttgaggccacacagctagatttttggtggaagtgggccttgaacacagtgtactttctgcagtttctgactgtaaaacccagtgtctgctctctgagttccatttccaagcccccctccatcttggacctatgtggtctccaccatattcacacaccaccaccaccacttgccaatgcctctcttaaagcaatatacccattcgttctcttattgggaactggatggatgaagccccaaattcagccccacccacagagaagccttcctacactcagcctctgtccacccttggcaaatctttcaagctctctcctccaggaaagtggggccccaactcagtcactccacccccttccaggtccctgaggctggttctactgtatccccatcacctccacaactccactcacccctgacggctccatccacctcaccagttggaaggcttgtggtttcagagaggagcaatgctggtcagcgctgcccagactccagtgtttacagatcaccagcatttacaaccaatccaatggccagaagcctcctctaacaagcccagaaggagttctgaaggggcagatgggggtgtgagtagtcggggagtcgggattgccagcaccctcacccttccttgggggcaagtagaggtgagaacactttccccacctccctccacagacactcctgaggacgctgcatcccacgcactgcctggtgcgtccatagagagaggatcaggtctcagcatttcatctgtgaaagaggcatggccctgggttagaaaggagggcaggagacatggaggaactggggggcacccagatggtgcagatggtttgcacacctgagcctgtctgtggtgaccattccgctcctctcccactaccctccaatctatcattccctactctctaaggccaaaatatcctgagcaaggctggcaaccccaccccaccatcccaaatgcaagcagccaggcccaggagttcctctggcccccacaggcatggagctcccagctggtgggtacagcttgagaggggggcagctccctcaggctaagctactgcccttcactgggccagccctgcctccagccctcacctctctcaccccaactctcccccaagcccctttctactcaacgggtgtagccactggtgctttgaagccttttgtttttataagatggtttttgcaaggggaccaggttctcttttcactgggaccttgcaaggaggggagtgctctcctggtttctgtgcaggcgggttgattaaagatggtgttttcttctcta

SEQ ID NO: 9 - DNA, 2503 bp, Homo sapiens
  source 1..2503: mol_type = genomic DNA
agtgtgtgaagtaaagggattaaaggctagtctcaggctggggatggctcctgtctatttcttctctctcagagactgcagatggcttttccctgccgcaggtccctgactgccaagactctggcctgcctcctggtgggcgtgagtttcttagcactgcagcagtggttcctccaggcgccaaggtccccgcgggaggagaggtccccgcaggaggagacgccagagggtcccaccgacgctcccgcggctgacgagccgccctcggagctcgtccccgggcccccgtgcgtggcgaacgcctcggcgaacgccacggccgacttcgagcagctgcccgcgcgcatccaggacttcctgcggtaccgccactgccgccacttcccgctgctttgggacgcaccggccaagtgcgccggcggccgaggcgtgttcctgctcctggcggtgaagtcggcgcctgagcactacgagcgacgcgagctcatccggcgcacgtgggggcaagagcgcagctacggcgggcggccagtgcgccgcctctttctattgggcaccccgggccccgaggacgaggcgcgcgcggagcggctggcggagctggtggcgctggaggcgcgcgagcacggcgacgtgctgcagtgggccttcgcggacaccttcctcaacctcacgctcaagcacctgcacttgctcgactggctggctgcacgctgcccgcacgcgcgctttctgctcagcggcgacgacgacgtgttcgtgcacaccgccaacgtagtccgcttcctgcaggcgcagccacccggccgccacctgttctccggccagctcatggagggctccgtgcccatccgcgacagctggagcaagtacttcgtgccgccgcagctcttccccgggtccgcttacccggtgtactgcagcggcggcggcttcctcctgtccggccccacggcccgggccctgcgcgcggccgcccgccacaccccgctcttccccatcgacgacgcctacatgggcatgtgtctggagcgcgccggcctggcgcccagcggccacgagggcatccgacccttcggcgtgcagctgcctggcgcacagcagtcctccttcgacccctgcatgtaccgcgagttgctgctagtgcaccgcttcgcgccctacgagatgctgctcatgtggaaggcgctgcacagccccgcgctcagctgtgaccggggacaccgggtctcctgaggccagttgggcggcttcagccccgggcctccaaccatgtccatgctgagaaggcagctttcccgctctgggtaccttacgtcctgcccagctctgtgcacctgaaccccagctgcgcactgaaatcagctggggtggggggtgtggaaaatgcctacatcctggctccatctcccgaagtttcgatttgattagtctggggtggacccagacatgttaagtattttttaagttcctccagtgatgcgaatgtgcagctaggcctgaggaccactcggctagactatctcttcatcctcgcaaagccagctccaccgccctctctgcaagaattccgggcccctcgctcccacactcgggtcctcttgagcagtggagcaagggagacctgggagcgtgggagccaggatcagcgccccctgccatgtgcctacaaatgtcagttgtgatttccactgtttacaagtgagtggagctggagctgggctgacagtatcaggtggatcccgcttccccctcccccaagaagtcagccaacacgcagctgaggcgcatgtggtggccttcttcccaccactaccccagtacaccgtgaggtagaaatcttcaccgtgcaaagtggaaaccagaggcccggtcagacagtgactaatccagggccgtggcattcccagacagcacaccactgtggtcccctccacactcaccccaaccaaagctaatggcctagttgggtcctgcccgccaataatcacccccacgggtcagagacaggctccttgccggggtctgggcctcaggctcagtgggccttggacaacccagcagggagttccggggagtccgaagtggagaaaggctggtgggaacatggaggccagtgttggggagcctgtggaggcaggtgtgtagaattgtgttcgggaggtgggggatctgagaccgaagtggacagtggttaagattgtggggccgggcgaggtggctcacgcctgtaatcccagcactttgggaggctgaggaggtcggatcatgaggtcaagagttcgagaccagcctggccaatatggtgaaaccccgtctctattgggagtacaaaaattagccggccatagtggctcgtgcctgtaatctcagctatttgggaggctgaggcaggagaatcacttgaacctgggaggcggaggttgcagtgagccgagatcgtgccactgcactccagcctgggcgacagagcaagactgcatctcaaaaaaaaaaaaaaaaa

SEQ ID NO: 10 - DNA, 867 bp, Homo sapiens
  source 1..867: mol_type = genomic DNA
gagtcctgagctgcgtcccggagcccacggtggtcatggctgccagagcgctctgcatgctggggctggtcctggccttgctgtcctccagctctgctgaggagtacgtgggcctgtctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcagaatgcaccttctgaggcacctccagctgcccccggccgggggatgcgaggctcggagcacccttgcccggctgtgattgctgccaggcactgttcatctcagcttttctgtccctttgctcccggcaagcgcttctgctgaaagttcatatctggagcctgatgtcttaacgaataaaggtcccatgctccacccgaggacagttcttcgtgcctgagactttctgaggttgtgctttatttctgctgcgtcgtgggagagggcgggagggtgtcaggggagagtctgcccaggcctcaagggcaggaaaagactccctaaggagctgcagtgcatgcaaggatattttgaatccagactggcacccacgtcacaggaaagcctaggaacactgtaagtgccgcttcctcgggaaagcagaaaaaatacatttcaggtagaagttttcaaaaatcacaagtctttcttggtgaagacagcaagccaataaaactgtcttccaaagtggtcctttatttcacaaccactctcgctactgttcaatacttgtactattcctgggttttgtttctttgtacagtaaacattatgaacaaacaggca

SEQ ID NO: 11 - DNA, 1579 bp, Homo sapiens
  source 1..1579: mol_type = genomic DNA
acccgaccgctgccggccgcgctcccgctgctcctgccgggtgatggaaaaccccagcccggccgccgccctgggcaaggccctctgcgctctcctcctggccactctcggcgccgccggccagcctcttgggggagagtccatctgttccgccagagccctggccaaatacagcatcaccttcacgggcaagtggagccagacggccttccccaagcagtaccccctgttccgcccccctgcgcagtggtcttcgctgctgggggccgcgcatagctccgactacagcatgtggaggaagaaccagtacgtcagtaacgggctgcgcgactttgcggagcgcggcgaggcctgggcgctgatgaaggagatcgaggcggcgggggaggcgctgcagagcgtgcacgcggtgttttcggcgcccgccgtccccagcggcaccgggcagacgtcggcggagctggaggtgcagcgcaggcactcgctggtctcgtttgtggtgcgcatcgtgcccagccccgactggttcgtgggcgtggacagcctggacctgtgcgacggggaccgttggcgggaacaggcggcgctggacctgtacccctacgacgccgggacggacagcggcttcaccttctcctcccccaacttcgccaccatcccgcaggacacggtgaccgagataacgtcctcctctcccagccacccggccaactccttctactacccgcggctgaaggccctgcctcccatcgccagggtgacactggtgcggctgcgacagagccccagggccttcatccctcccgccccagtcctgcccagcagggacaatgagattgtagacagcgcctcagttccagaaacgccgctggactgcgaggtctccctgtggtcgtcctggggactgtgcggaggccactgtgggaggctcgggaccaagagcaggactcgctacgtccgggtccagcccgccaacaacgggagcccctgccccgagctcgaagaagaggctgagtgcgtccctgataactgcgtctaagaccagagccccgcagcccctggggccccccggagccatggggtgtcgggggctcctgtgcaggctcatgctgcaggcggccgagggcacagggggtttcgcgctgctcctgaccgcggtgaggccgcgccgaccatctctgcactgaagggccctctggtggccggcacgggcattgggaaacagcctcctcctttcccaaccttgcttcttaggggcccccgtgtcccgtctgctctcagcctcctcctcctgcaggataaagtcatccccaaggctccagctactctaaattatgtctccttataagttattgctgctccaggagattgtccttcatcgtccaggggcctggctcccacgtggttgcagatacctcagacctggtgctctaggctgtgctgagcccactctcccgagggcgcatccaagcgggggccacttgagaagtgaataaatggggcggtttcggaagcgtcagtgtttccatgttatggatctctctgcgtttgaataaagactatctctgttgctcacaaa

SEQ ID NO: 12 - DNA, 1603 bp, Homo sapiens
  source 1..1603: mol_type = genomic DNA
aaggcactctggcacccagttttggaactgcagttttaaaagtcataaattgaatgaaaatgatagcaaaggtggaggtttttaaagagctatttataggtccctggacagcatcttttttcaattaggcagcaacctttttgccctatgccgtaacctgtgtctgcaacttcctctaattgggaaatagttaagcagattcatagagctgaatgataaaattgtactacgagatgcactgggactcaacgtgaccttatcaagtgagcaggcttggtgcatttgacacttcatgatatcagccaaagtggaactaaaaacagctcctggaagaggactatgacatcatcaggttgggagtctccagggacagcggaccctttggaaaaggactagaaagtgtgaaatctattagtcttcgatatgaaattctctgtctctgtaaaagcatttcatatttacaagacacaggcctactcctagggcagcaaaaagtggcaacaggcaagcagagggaaaagagatcatgaggcatttcagagtgcactgtcttttcatatatttctcaatgccgtatgtttggttttattttggccaagcataacaatctgctcaagaaaaaaaaatctggagaaaacaaaggtgcctttgccaatgttatgtttctttttgacaagccctgagatttctgaggggaattcacataaatgggatcaggtcattcatttacgttgtgtgcaaatatgatttaaagatacaacctttgcagagagcatgctttcctaagggtaggcacgtggaggactaagggtaaagcattcttcaagatcagttaatcaagaaaggtgctctttgcattctgaaatgcccttgttgcaaatattggttatattgattaaatttacacttaatggaaacaacctttaacttacagatgaacaaacccacaaaagcaaaaaatcaaaagccctacctatgatttcatattttctgtgtaactggattaaaggattcctgcttgcttttgggcataaatgataatggaatatttccaggtattgtttaaaatgagggcccatctacaaattcttagcaatactttggataattctaaaattcagctggacattgtctaattgttttttatatacatctttgctagaatttcaaattttaagtatgtgaatttagttaattagctgtgctgatcaattcaaaaacattactttcctaaattttagactatgaaggtcataaattcaacaaatatatctacacatacaattatagattgtttttcattataatgtcttcatcttaacagaattgtctttgtgattgtttttagaaaactgagagttttaattcataattacttgatcaaaaaattgtgggaacaatccagcattaattgtatgtgattgtttttatgtacataaggagtcttaagcttggtgccttgaagtcttttgtacttagtcccatgtttaaaattactactttatatctaaagcatttatgtttttcaattcaatttacatgatgctaattatggcaattataacaaatattaaagatttcgaaatagaaaaaaaaaaaaaaa

SEQ ID NO: 13 - DNA, 1530 bp, Homo sapiens
  source 1..1530: mol_type = genomic DNA
gtgaggacaccgctttacaacgatgcagggggccccatgtcaccctcacccatgggaagtttgacttggtggactcagccaagccacagaggtctaacgcttctctgcggtgatttcaggctgccctggcagaaagcacagtgcctgcagacatgctgtcactgctccacgcatcaacgctggcagtccttggggctctgtgtgtatatggtgcaggtcacctagagcaacctcaaatttccagtactaaaacgctgtcaaaaacagcccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgttggagggaaattataagaaactctttggcagtggaacaacacttgttgtcacagataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaaaagtcactggacaaagaacacagatgtatcgtcagacatgagaataataaaaacggagttgatcaagaaattatctttcctccaataaagacagatgtcatcacaatggatcccaaagacaattgttcaaaagatgcaaatgatacactactgctgcagctcacaaacacctctgcatattacatgtacctcctcctgctcctcaagagtgtggtctattttgccatcatcacctgctgtctgcttagaagaacggctttctgctgcaatggagagaaatcataacagacggtggcacaaggaggccatcttttcctcatcggttattgtccctagaagcgtcttctgaggatctagttgggctttctttctgggtttgggccatttcagttctcatgtgtgtactattctatcattattgtataacggttttcaaaccagtgggcacacagagaacctcactctgtaataacaatgaggaatagccacggcgatctccagcaccaatctctccatgttttccacagctcctccagccaacccaaatagcgcctgctatagtgtagacatcctgcggcttctagccttgtccctctcttagtgttctttaatcagataactgcctggaagcctttcattttacacgccctgaagcagtcttctttgctagttgaattatgtggtgtgtttttccgtaataagcaaaataaatttaaaaaaatgaaaagtt

SEQ ID NO: 14 - DNA, 634 bp, Homo sapiens
  source 1..634: mol_type = genomic DNA
aacgctaacctggtccggagcgagtctgggtctcagccccgcgaacagcctttcacgagtcttcaagctttcaggctatcttctagtcaagatgagtgataagccagacttgtcggaagtggagaagtttgacaggtcaaaactgaagaaaactaatactgaagaaaaaaatactcttccctcaaaggaaactatccagcaagagaaagagtgtgttcaaacatcataaaatggggatcgcctcccaacagcagatttcgacattacctgagagtcttgattttaggcttgttttttgtaaacccatgtgtttgtagagattttaggcgtcttcggatatcttctcacctatgttccctggctaagaagtcagaggtagccaatgtttccttaaattcatttttaaacttaccattggtgcatatgttccagatggcagatgctgtcaataatctcaccattgatgacctttgtgtatgtagttcttgcatcctatactggataagcctgttttaacctgctatgatgggtgcttccattgcttcataatcttcatgaagttgcatgcttttgcagcttttcacagtttatttgcatttctaatgtagtaataaagtaaccaatataatcatta

SEQ ID NO: 15 - DNA, 4904 bp, Homo sapiens
  source 1..4904: mol_type = genomic DNA
atccgctctaaacaacctcatcaaaactactttctggtcagagagaagcaataattattattaacatttattaacgatcaataaacttgatcgcattatggccagcactattaaggaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaaggaactctcctgatgaatgcagtgtggccaaaggcgggaagatggtgggcagcccagacaccgttgggatgaactacggcagctacatggaggagaagcacatgccacccccaaacatgaccacgaacgagcgcagagttatcgtgccagcagatcctacgctatggagtacagaccatgtgcggcagtggctggagtgggcggtgaaagaatatggccttccagacgtcaacatcttgttattccagaacatcgatgggaaggaactgtgcaagatgaccaaggacgacttccagaggctcacccccagctacaacgccgacatccttctctcacatctccactacctcagagagactcctcttccacatttgacttcagatgatgttgataaagccttacaaaactctccacggttaatgcatgctagaaacacagggggtgcagcttttattttcccaaatacttcagtatatcctgaagctacgcaaagaattacaactaggccagatttaccatatgagccccccaggagatcagcctggaccggtcacggccaccccacgccccagtcgaaagctgctcaaccatctccttccacagtgcccaaaactgaagaccagcgtcctcagttagatccttatcagattcttggaccaacaagtagccgccttgcaaatccaggcagtggccagatccagctttggcagttcctcctggagctcctgtcggacagctccaactccagctgcatcacctgggaaggcaccaacggggagttcaagatgacggatcccgacgaggtggcccggcgctggggagagcggaagagcaaacccaacatgaactacgataagctcagccgcgccctccgttactactatgacaagaacatcatgaccaaggtccatgggaagcgctacgcctacaagttcgacttccacgggatcgcccaggccctccagccccaccccccggagtcatctctgtacaagtacccctcagacctcccgtacatgggctcctatcacgcccacccacagaagatgaactttgtggcgccccaccctccagccctccccgtgacatcttccagtttttttgctgccccaaacccatactggaattcaccaactgggggtatataccccaacactaggctccccaccagccatatgccttctcatctgggcacttactactaaagacctggcggaggcttttcccatcagcgtgcattcaccagcccatcgccacaaactctatcggagaacatgaatcaaaagtgcctcaagaggaatgaaaaaagctttactggggctggggaaggaagccggggaagagatccaaagactcttgggagggagttactgaagtcttactacagaaatgaggaggatgctaaaaatgtcacgaatatggacatatcatctgtggactgaccttgtaaaagacagtgtatgtagaagcatgaagtcttaaggacaaagtgccaaagaaagtggtcttaagaaatgtataaactttagagtagagtttggaatcccactaatgcaaactgggatgaaactaaagcaatagaaacaacacagttttgacctaacataccgtttataatgccattttaaggaaaactacctgtatttaaaaatagaaacatatcaaaaacaagagaaaagacacgagagagactgtggcccatcaacagacgttgatatgcaactgcatggcatgtgctgttttggttgaaatcaaatacattccgtttgatggacagctgtcagctttctcaaactgtgaagatgacccaaagtttccaactcctttacagtattaccgggactatgaactaaaaggtgggactgaggatgtgtatagagtgagcgtgtgattgtagacagaggggtgaagaaggaggaggaagaggcagagaaggaggagaccagggctgggaaagaaacttctcaagcaatgaagactggactcaggacatttggggactgtgtacaatgagttatggagactcgagggttcatgcagtcagtgttataccaaacccagtgttaggagaaaggacacagcgtaatggagaaaggggaagtagtagaattcagaaacaaaaatgcgcatctctttctttgtttgtcaaatgaaaattttaactggaattgtctgatatttaagagaaacattcaggacctcatcattatgtgggggctttgttctccacagggtcaggtaagagatggccttcttggctgccacaatcagaaatcacgcaggcattttgggtaggcggcctccagttttcctttgagtcgcgaacgctgtgcgtttgtcagaatgaagtatacaagtcaatgtttttccccctttttatataataattatataacttatgcatttatacactacgagttgatctcggccagccaaagacacacgacaaaagagacaatcgatataatgtggccttgaattttaactctgtatgcttaatgtttacaatatgaagttattagttcttagaatgcagaatgtatgtaataaaataagcttggcctagcatggcaaatcagatttatacaggagtctgcatttgcactttttttagtgactaaagttgcttaatgaaaacatgtgctgaatgttgtggattttgtgttataatttactttgtccaggaacttgtgcaagggagagccaaggaaataggatgtttggcacccaaatggcgtcagcctctccaggtccttcttgcctcccctcctgtcttttatttctagccccttttggaacagaaggaccccgggtttcacattggagcctccatatttatgcctggaatggaaagaggcctatgaagctggggttgtcattgagaaattctagttcagcacctggtcacaaatcacccttaattcctgctatgattaaaatacatttgttgaacagtgaacaagctaccactcgtaaggcaaactgtattattactggcaaataaagcgtcatggatagctgcaatttctcactttacagaaacaagggataacgtctagatttgctgcggggtttctctttcaggagctctcactaggtagacagctttagtcctgctacatcagagttacctgggcactgtggcttgggattcactagccctgagcctgatgttgctggctatcccttgaagacaatgtttatttccataatctagagtcagtttccctgggcatcttttctttgaatcacaaatgctgccaaccttggtccaggtgaaggcaactcaaaaggtgaaaatacaaggtgaccgtgcgaaggcgctagccgaaacatcttagctgaataggtttctgaactggcccttttcatagctgtttcagggcctgtttttttcacgttgcagtccttttgctatgattatgtgaagttgccaaacctctgtgctgtggatgttttggcagtgggctttgaagtcggcaggacacgattaccaatgctcctgacaccccgtgtcatttggattagacggagcccaaccatccatcattttgcagcagcctgggaaggcccacaaagtgcccgtatctccttagggaaaataaataaatacaatcatgaaagctggcagttaggctgacccaaactgtgctaatggaaaagatcagtcatttttattttggaatgcaaagtcaagacacacctacattcttcatagaaatacacatttacttggataatcactcagttctctcttcaagactgtctcatgagcaagatcataaaaacaagacatgattatcatattcaattttaacagatgttttccattagatccctcaaccctccacccccagtccaggttattagcaagtcttatgagcaactgggataattttggataacatgataatactgagttccttcaaatacataattcttaaattgtttcaaaatggcattaactctctgttactgttgtaatctaattccaaagccccctccaggtcatattcataattgcatgaaccttttctctctgtttgtccctgtctcttggcttgccctgatgtatactcagactcctgtacaatcttactcctgctggcaagagatttgtcttcttttcttgtcttcaattggctttcgggccttgtatgtggtaaaatcaccaaatcacagtcaagactgtgtttttgttcctagtttgatgcccttatgtcccggaggggttcacaaagtgctttgtcaggactgctgcagttagaaggctcactgcttctcctaagccttctgcacagatgtggcacctgcaacccaggagcaggagccggaggagctgccctctgacagcaggtgcagcagagatggctacagctcaggagctgggaaggtgatggggcacagggaaagcacagatgttctgcagcgccccaaagtgacccattgcctggagaaagagaagaaaatattttttaaaaagctagtttatttagcttctcattaattcattcaaataaagtcgtgaggtgactaattagagaataaaaattactttggactactcaaaaa

SEQ ID NO: 16 - DNA, 1416 bp, Homo sapiens
  source 1..1416: mol_type = genomic DNA
aggcagcaggctggagctcagcccagcagtggaatccaggagcccagaggtggccgggtgctgacgtgatggccacagcaggaaatccctggggctggttcctggggtacctcatccttggtgtcgcaggatcgctcgtctctggtagctgcagccaaatcataaacggcgaggactgcagcccgcactcgcagccctggcaggcggcactggtcatggaaaacgaattgttctgctcgggcgtcctggtgcatccgcagtgggtgctgtcagccgcacactgtttccagaactcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacggcagaatgcctaccgtgctgcagtgcgtgaacgtgtcggtggtgtctgaggaggtctgcagtaagctctatgacccgctgtaccaccccagcatgttctgcgccggcggagggcaagaccagaaggactcctgcaacggtgactctggggggcccctgatctgcaacgggtacttgcagggccttgtgtctttcggaaaagccccgtgtggccaagttggcgtgccaggtgtctacaccaacctctgcaaattcactgagtggatagagaaaaccgtccaggccagttaactctggggactgggaacccatgaaattgacccccaaatacatcctgcggaaggaattcaggaatatctgttcccagcccctcctccctcaggcccaggagtccaggcccccagcccctcctccctcaaaccaagggtacagatccccagcccctcctccctcagacccaggagtccagaccccccagcccctcctccctcagacccaggagtccagcccctcctccctcagacccaggagtccagaccccccagcccctcctccctcagacccaggagtccagcccctcctccctcagacccaggagtccagaccccccagcccctcctccctcagacccaggggtccaggcccccaacccctcctccctcagactcagaggtccaggcccccaacccctccttccccagacccagaggtccaggtcccagcccctcctccctcagacccagcggtccaatgccacctagactctccctgtacacagtgcccccttgtggcacgttgacccaaccttaccagttggtttttcattttttgtccctttcccctagatccagaaataaagtctaagagaagcgca

SEQ ID NO: 17 - DNA, 1651 bp, Homo sapiens
  source 1..1651: mol_type = genomic DNA
ataaccatctagttccgagtacaaactggagacagaaataaatattaaagaaatcatagaccgaccaggtaaaggcaaagggatgaattcctacttcactaacccttccttatcctgccacctcgccgggggccaggacgtcctccccaacgtcgccctcaattccaccgcctatgatccagtgaggcatttctcgacctatggagcggccgttgcccagaaccggatctactcgactcccttttattcgccacaggagaatgtcgtgttcagttccagccgggggccgtatgactatggatctaattccttttaccaggagaaagacatgctctcaaactgcagacaaaacaccttaggacataacacacagacctcaatcgctcaggattttagttctgagcagggcaggactgcgccccaggaccagaaagccagtatccagatttacccctggatgcagcgaatgaattcgcacagtggggtcggctacggagcggaccggaggcgcggccgccagatctactcgcggtaccagaccctggaactggagaaggaatttcacttcaatcgctacctaacgcggcgccggcgcatcgagatcgccaacgcgctttgcctgaccgagcgacagatcaaaatctggttccagaaccgccggatgaagtggaaaaaagaatctaatctcacatccactctctcggggggcggcggaggggccaccgccgacagcctgggcggaaaagaggaaaagcgggaagagacagaagaggagaagcagaaagagtgaccaggactgtccctgccacccctctctccctttctccctcgctccccaccaactctcccctaatcacacactctgtatttatcactggcacaattgatgtgttttgattccctaaaacaaaattagggagtcaaacgtggacctgaaagtcagctctggaccccctccctcaccgcacaactctctttcaccacgcgcctcctcctcctcgctcccttgctagctcgttctcggcttgtctacaggcccttttccccgtccaggccttgggggctcggaccctgaactcagactctacagattgccctccaagtgaggacttggctcccccactccttcgacgcccccacccccgccccccgtgcagagagccggctcctgggcctgctggggcctctgctccagggcctcagggcccggcctggcagccggggagggccggaggcccaaggagggcgcgccttggccccacaccaacccccagggcctccccgcagtccctgcctagcccctctgccccagcaaatgcccagcccaggcaaattgtatttaaagaatcctgggggtcattatggcattttacaaactgtgaccgtttctgtgtgaagatttttagctgtatttgtggtctctgtatttatatttatgtttagcaccgtcagtgttcctatccaatttcaaaaaaggaaaaaaaagagggaaaattacaaaaagagagaaaaaaagtgaatgacgtttgtttagccagtaggagaaaataaataaataaataaatcccttcgtgttaccctcctgtataaatccaacctctgggtccgttctcgaatatttaataaaactgatattatttttaaaacttta

SEQ ID NO: 18 - DNA, 1464 bp, Homo sapiens
  source 1..1464: mol_type = genomic DNA
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 19 - DNA, 2081 bp, Homo sapiens
  source 1..2081: mol_type = genomic DNA
atacctaccagggacctagttcagctacgtcctaaacagtaccaggcaggtggtgacttgagaactctgtgcctggtttctgaggactgtttcaccatgcagtggctaatgaggttccggaccctctggggcatccacaaatccttccacaacatccaccctgccccttcacagctgcgctgccggtctttatcagaatttggagccccaagatggaatgactatgaagtaccggaggaatttaactttgcaagttatgtactggactactgggctcaaaaggagaaggagggcaagagaggtccaaatccagctttttggtgggtgaatggccaaggggatgaagtaaagtggagcttcagagagatgggagacctaacccgccgtgtagccaacgtcttcacacagacctgtggcctacaacagggagaccatctggccttgatgctgcctcgagttcctgagtggtggctggtggctgtgggctgcatgcgaacagggatcatcttcattcctgcgaccatcctgttgaaggccaaagacattctctatcgactacagttgtctaaagccaagggcattgtgaccatagatgcccttgcctcagaggtggactccatagcttctcagtgcccctctctgaaaaccaagctcctggtgtctgatcacagccgtgaagggtggctggacttccgatcgctggttaaatcagcatccccagaacacacctgtgttaagtcaaagaccttggacccaatggtcatcttcttcaccagtgggaccacaggcttccccaagatggcaaaacactcccatgggttggccttacaaccctccttcccaggaagtaggaaattacggagcctgaagacatctgatgtctcctggtgcctgtcggactcaggatggattgtggctaccatttggaccctggtagaaccatggacagcgggttgtacagtctttatccaccatctgccacagtttgacaccaaggtcatcatacagacattgttgaaataccccattaaccacttttggggggtatcatctatatatcgaatgattctgcagcaggatttcaccagcatcaggttccctgccctggagcactgctatactggcggggaggtcgtgttgcccaaggatcaggaggagtggaaaagacggacgggccttctgctctacgagaactatgggcagtcggaaacgggactaatttgtgccacctactggggaatgaagatcaagccgggtttcatggggaaggccactccaccctacgacgtccaggtcattgatgacaagggcagcatcctgccacctaacacagaaggaaacattggcatcagaatcaaacctgtcaggcctgtgagcctcttcatgtgctatgagggtgacccagagaagacagctaaagtggaatgtggggacttctacaacactggggacagaggtaagatggatgaagagggctacatttgtttcctggggaggagtgatgacatcattaatgcctctgggtatcgcatcgggcctgcagaggttgaaagcgctttggtggagcacccagcggtggcggagtcagccgtggtgggcagcccagacccgattcgaggggaggtggtgaaggcctttattgtcctgaccccacagttcctgtcccatgacaaggatcagctgaccaaggaactgcagcagcatgtcaagtcagtgacagccccatacaagtacccaaggaaggtggagtttgtctcagagctgccaaaaaccatcactggcaagattgaacggaaggaacttcggaaaaaggagactggtcagatgtaatcggcagtgaactcagaacgcactgcacacctaaggcaaatccctggccactttagtctccccactatggtgaggacgagggtggggcattgagagtgttgatttgggaaagtatcaggagtgccatgattccaatgttttccttcttttaaattaaattcagttgctctgcttcctccaagtcctctgtatctttagaatttcccaggtgagcactcataacgcaagtaataaaatactgatatcaacaa

SEQ ID NO: 20 - DNA, 4108 bp, Homo sapiens
  source 1..4108: mol_type = genomic DNA
agtttccttcagcggggcactgggaagcgccatggcactgcagggcatctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtgttctctcaaaaattggcagaagtggtgagaatccgtatgccccgctgaatctcctggctgactttgctggtggtggccttatgtgtgcactgggcattataatggctctttttgaccgcacacgcactggcaagggtcaggtcattgatgcaaatatggtggaaggaacagcatatttaagttcttttctgtggaaaactcagaaattgagtctgtgggaagcacctcgaggacagaacatgttggatggtggagcacctttctatacgacttacaggacagcagatggggaattcatggctgttggagcaatagaaccccagttctacgagctgctgatcaaaggacttggactaaagtctgatgaacttcccaatcagatgagcatggatgattggccagaaatgaagaagaagtttgcagatgtatttgcagagaagacgaaggcagagtggtgtcaaatctttgacggcacagatgcctgtgtgactccggttctgacttttgaggaggttgttcatcatgatcacaacaaggaacggggctcgtttatcaccagtgaggagcaggacgtgagcccccgccctgcacctctgctgttaaacaccccagccatcccttctttcaaaagggatcctttcataggagaacacactgaggagatacttgaagaatttggattcagccgcgaagagatttatcagcttaactcagataaaatcattgaaagtaataaggtaaaagctagtctctaacttccaggcccacggctcaagtgaatttgaatactgcatttacagtgtagagtaacacataacattgtatgcatggaaacatggaggaacagtattacagtgtcctaccactctaatcaagaaaagaattacagactctgattctacagtgatgattgaattctaaaaatggttatcattagggcttttgatttataaaactttgggtacttatactaaattatggtagttattctgccttccagtttgcttgatatatttgttgatattaagattcttgacttatattttgaatgggttctagtgaaaaaggaatgatatattcttgaagacatcgatatacatttatttacactcttgattctacaatgtagaaaatgaggaaatgccacaaattgtatggtgataaaagtcacgtgaaacagagtgattggttgcatccaggccttttgtcttggtgttcatgatctccctctaagcacattccaaactttagcaacagttatcacactttgtaatttgcaaagaaaagtttcacctgtattgaatcagaatgccttcaactgaaaaaaacatatccaaaataatgaggaaatgtgttggctcactacgtagagtccagagggacagtcagttttagggttgcctgtatccagtaactcggggcctgtttccccgtgggtctctgggctgtcagctttcctttctccatgtgtttgatttctcctcaggctggtagcaagttctggatcttatacccaacacacagcaacatccagaaataaagatctcaggaccccccagcaagtcgttttgtgtctccttggactgagttaagttacaagcctttcttatacctgtctttgacaaagaagacgggattgtctttacataaaaccagcctgctcctggagcttccctggactcaacttcctaaaggcatgtgaggaaggggtagattccacaatctaatccgggtgccatcagagtagagggagtagagaatggatgttgggtaggccatcaataaggtccattctgcgcagtatctcaactgccgttcaacaatcgcaagaggaaggtggagcaggtttcttcatcttacagttgagaaaacagagactcagaagggcttcttagttcatgtttcccttagcgcctcagtgattttttcatggtggcttaggccaaaagaaatatctaaccattcaatttataaataattaggtccccaacgaattaaatattatgtcctaccaacttattagctgcttgaaaaatataatacacataaataaaaaaatatatttttcatttctatttcattgttaatcacaactacttactaaggagatgtatgcacctattggacactgtgcaacttctcacctggaatgagattggacactgctgccctcattttctgctccatgttggtgtccatatagtacttgattttttatcagatggcctggaaaacccagtctcacaaaaatatgaaattatcagaaggattatagtgcaatcttatgttgaaagaatgaactacctcactagtagttcacgtgatgtctgacagatgttgagtttcattgtgtttgtgtgttcaaatttttaaatattctgagatactcttgtgaggtcactctaatgccctgggtgccttggcacagttttagaaataccagttgaaaatatttgctcaggaatatgcaactaggaaggggcagaatcagaatttaagctttcatattctagccttcagtcttgttcttcaaccatttttaggaactttcccataaggttatgttttccagcccaggcatggaggatcacttgaggccaagagttcgagaccagcctggggaacttggctggacctccgtttctacgaaataaaaataaaaaaattatccaggtatggtggtgtgtgcctgtagtcctatctactcaagggtggggcaggaggatcacttgagcccaggaatttgaggccacagtgaattaggattgcaccactgcactctagcccaggcaacagaacaagaacctgtctctaaataaataaataaaaataataataataaaaaagatgttttccctacaactcagacttttcatttgaactcggtccagcaaggaaaatataacccactcgaagtctttaaaacagaggaaatttaatataaagaattccactggtgacgaaagagcagagaagcccagaagatagtgaggcaaccctgataggaacataactaggaagccaagaccactcctatggttgcaggggtgatgggaaagctggtgtacttggacccagaagccaaagttgctgcacccaccttggagacatagacactggcagtaatacctcagggagaagaaagaaatctagggaaatatcctggcttctttccttctctctctcccctagtcttcctaccagtgtctcccattagccaaatctacctagaagccagaaaacaagggaaccctggaaatgtagccccataagataaagagcaccaaaggaaatagatctgagcagacaggcagcacaaaatgcagtgtgtatggtttattcactcagtaattcctttagcaaatgtttattgaggatctactaggtgccaggtatcatgatacttgctggggataccataatgaacaaaacagacctgttctccgctcttgaggaaatcaaagacaaacacaggatatggaataaacccagaattatctcattgtaaaatgtgttaagtaccacgaggagaaatatcagggcgatctgacacagctaatgatttgaagaagggtgtgacctgccaccattttaaatctagttatttcactcctgagctgtgtgtgtggaaaacttgtagtaaaaaatagaatgtctatatttataaaaagtttatgaaaaga

SEQ ID NO: 21 - DNA, 10667 bp, Homo sapiens
  source 1..10667: mol_type = genomic DNA
agcgccccctccgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaacaaaaaaaaaaagcaaaaacaaaacaaaaaataagccaaaaaaccttgctagtgttttttcctcaaaaataaataaataaataaataaatacgtacatacatacacacatacatacaaacatatagaaatccccaaagaggccaatagtgacgagaaggtgaaaattgcaggcccatggggagttactgattttttcatctcctccctccacgggagactttattttctgccaatggctattgccattagagggcagagtgaccccagagctgagttgggcaggggggtggacagagaggagaggacaaggagggcaatggagcatcagtacctgcccacagccttggtccctgggggctagactgctcaactgtggagcaattcattatactgaaaatgtgcttgttgttgaaaatttgtctgcatgttaatgcctcacccccaaacccttttctctctcactctctgcctccaacttcagattgactttcaatagtttttctaagacctttgaactgaatgttctcttcagccaaaacttggcgacttccacagaaaagtctgaccactgagaagaaggagagcagagatttaaccctttgtaaggccccatttggatccaggtctgctttctcatgtgtgagtcagggaggagctggagccagaggagaagaaaatgatagcttggctgttctcctgcttaggacactgactgaatagttaaactctcactgccactaccttttccccacctttaaaagacctgaatgaagttttctgccaaactccgtgaagccacaagcaccttatgtcctcccttcagtgttttgtgggcctgaatttcatcacactgcatttcagccatggtcatcaagcctgtttgcttcttttgggcatgttcacagattctctgttaagagcccccaccaccaagaaggttagcaggccaacagctctgacatctatctgtagatgccagtagtcacaaagatttcttaccaactctcagatcgctggagcccttagacaaactggaaagaaggcatcaaagggatcaggcaagctgggcgtcttgcccttgtcccccagagatgataccctcccagcaagtggagaagttctcacttccttctttagagcagctaaaggggctacccagatcagggttgaagagaaaactcaattaccagggtgggaagaatgaaggcactagaaccagaaaccctgcaaatgctcttcttgtcacccagcatatccacctgcagaagtcatgagaagagagaaggaacaaagaggagactctgactactgaattaaaatcttcagcggcaaagcctaaagccagatggacaccatctggtgagtttactcatcatcctcctctgctgctgattctgggctctgacattgcccatactcactcagattccccacctttgttgctgcctcttagtcagagggaggccaaaccattgagactttctacagaaccatggcttctttcggaaaggtctggttggtgtggctccaatactttgccacccatgaactcagggtgtgccctgggacactggttttatatagtcttttggcacacctgtgttctgttgacttcgttcttcaagcccaagtgcaagggaaaatgtccacctactttctcatcttggcctctgcctccttacttagctcttaatctcatctgttgaactcaagaaatcaagggccagtcatcaagctgcccattttaattgattcactctgtttgttgagaggatagtttctgagtgacatgatatgatccacaagggtttccttccctgatttctgcattgatattaatagccaaacgaacttcaaaacagctttaaataacaagggagaggggaacctaagatgagtaatatgccaatccaagactgctggagaaaactaaagctgacaggttccctttttggggtgggatagacatgttctggttttctttattattacacaatctggctcatgtacaggatcacttttagctgttttaaacagaaaaaaatatccaccactcttttcagttacactaggttacattttaataggtcctttacatctgttttggaatgattttcatcttttgtgatacacagattgaattatatcattttcatatctctccttgtaaatactagaagctctcctttacatttctctatcaaatttttcatctttatgggtttcccaattgtgactcttgtcttcatgaatatatgtttttcatttgcaaaagccaaaaatcagtgaaacagcagtgtaattaaaagcaacaactggattactccaaatttccaaatgacaaaactagggaaaaatagcctacacaagcctttaggcctactctttctgtgcttgggtttgagtgaacaaaggagattttagcttggctctgttctcccatggatgaaaggaggaggattttttttttcttttggccattgatgttctagccaatgtaattgacagaagtctcattttgcatgcgctctgctctacaaacagagttggtatggttggtatactgtactcacctgtgagggactggccactcagacccacttagctggtgagctagaagatgaggatcactcactggaaaagtcacaaggaccatctccaaacaagttggcagtgctcgatgtggacgaagagtgaggaagagaaaaagaaggagcaccagggagaaggctccgtctgtgctgggcagcagacagctgccaggatcacgaactctgtagtcaaagaaaagagtcgtgtggcagtttcagctctcgttcattgggcagctcgcctaggcccagcctctgagctgacatgggagttgttggattctttgtttcatagctttttctatgccataggcaatattgttgttcttggaaagtttattatttttttaactcccttactctgagaaagggatattttgaaggactgtcatatatctttgaaaaaagaaaatctgtaatacatatatttttatgtatgttcactggcactaaaaaatatagagagcttcattctgtcctttgggtagttgctgaggtaattgtccaggttgaaaaataatgtgctgatgctagagtccctctctgtccatactctacttctaaatacatataggcatacatagcaagttttatttgacttgtactttaagagaaaatatgtccaccatccacatgatgcacaaatgagctaacattgagcttcaagtagcttctaagtgtttgtttcattaggcacagcacagatgtggcctttccccccttctctcccttgatatctggcagggcataaaggcccaggccacttcctctgccccttcccagccctgcaccaaagctgcatttcaggagactctctccagacagcccagtaactacccgagcatggcccctgcatagccctggaaaaataagaggctgactgtctacgaattatcttgtgccagttgcccaggtgagagggcactgggccaagggagtggttttcatgtttgacccactacaaggggtcatgggaatcaggaatgccaaagcaccagatcaaatccaaaacttaaagtcaaaataagccattcagcatgttcagtttcttggaaaaggaagtttctacccctgatgcctttgtaggcagatctgttctcaccattaatctttttgaaaatcttttaaagcagtttttaaaaagagagatgaaagcatcacattatataaccaaagattacattgtacctgctaagataccaaaattcataagggcagggggggagcaagcattagtgcctctttgataagctgtccaaagacagactaaaggactctgctggtgactgacttataagagctttgtgggtttttttttccctaataatatacatgtttagaagaattgaaaataatttcgggaaaatgggattatgggtccttcactaagtgattttataagcagaactggctttccttttctctagtagttgctgagcaaattgttgaagctccatcattgcatggttggaaatggagctgttcttagccactgtgtttgctagtgcccatgttagcttatctgaagatgtgaaacccttgctgataagggagcatttaaagtactagattttgcactagagggacagcaggcagaaatccttatttctgcccactttggatggcacaaaaagttatctgcagttgaaggcagaaagttgaaatacattgtaaatgaatatttgtatccatgtttcaaaattgaaatatatatatatatatatatatatatatatatatatatatagtgtgtgtgtgtgttctgatagctttaactttctctgcatctttatatttggttccagatcacacctgatgccatgtacttgtgagagaggatgcagttttgttttggaagctctctcagaacaaacaagacacctggattgatcagttaactaaaagttttctcccctattgggtttgacccacaggtcctgtgaaggagcagagggataaaaagagtagaggacatgatacattgtactttactagttcaagacagatgaatgtggaaagcataaaaactcaatggaactgactgagatttaccacagggaaggcccaaacttggggccaaaagcctacccaagtgattgaccagtggccccctaatgggacctgagctgttggaagaagagaactgttccttggtcttcaccatccttgtgagagaagggcagtttcctgcattggaacctggagcaagcgctctatctttcacacaaattccctcacctgagattgaggtgctcttgttactgggtgtctgtgtgctgtaattctggttttggatatgttctgtaaagattttgacaaatgaaaatgtgtttttctctgttaaaacttgtcagagtactagaagttgtatctctgtaggtgcaggtccatttctgcccacaggtagggtgtttttctttgattaagagattgacacttctgttgcctaggacctcccaactcaaccatttctaggtgaaggcagaaaaatccacattagttactcctcttcagacatttcagctgagataacaaatcttttggaattttttcacccatagaaagagtggtagatatttgaatttagcaggtggagtttcatagtaaaaacagcttttgactcagctttgatttatcctcatttgatttggccagaaagtaggtaatatgcattgattggcttctgattccaattcagtatagcaaggtgctaggttttttcctttccccacctgtctcttagcctggggaattaaatgagaagccttagaatgggtggcccttgtgacctgaaacacttcccacataagctacttaacaagattgtcatggagctgcagattccattgcccaccaaagactagaacacacacatatccatacaccaaaggaaagacaattctgaaatgctgtttctctggtggttccctctctggctgctgcctcacagtatgggaacctgtactctgcagaggtgacaggccagatttgcattatctcacaaccttagcccttggtgctaactgtcctacagtgaagtgcctggggggttgtcctatcccataagccacttggatgctgacagcagccaccatcagaatgacccacgcaaaaaaaagaaaaaaaaaattaaaaagtcccctcacaacccagtgacacctttctgctttcctctagactggaacattgattagggagtgcctcagacatgacattcttgtgctgtccttggaattaatctggcagcaggagggagcagactatgtaaacagagataaaaattaattttcaatattgaaggaaaaaagaaataagaagagagagagaaagaaagcatcacacaaagattttcttaaaagaaacaattttgcttgaaatctctttagatggggctcatttctcacggtggcacttggcctccactgggcagcaggaccagctccaagcgctagtgttctgttctctttttgtaatcttggaatcttttgttgctctaaatacaattaaaaatggcagaaacttgtttgttggactacatgtgtgactttgggtctgtctctgcctctgctttcagaaatgtcatccattgtgtaaaatattggcttactggtctgccagctaaaacttggccacatcccctgttatggctgcaggatcgagttattgttaacaaagagacccaagaaaagctgctaatgtcctcttatcattgttgttaatttgttaaaacataaagaaatctaaaatttca

SEQ ID NO: 22 - DNA, 2852 bp, Homo sapiens
  source 1..2852: mol_type = genomic DNA
gaacagccagcgctggaggagcgccgggagactctgccgtcggtgcgtgcgcggacacgcacccgtcccccttggtctcgccgccagccatggccgccgctacggcctccccccgcagcctccttgttctcctccaggtggtagtgctcgctctggcgcagattagaggtccaccgggagagcggggccccccgggtcccccgggaccgccgggagtgcctggatccgacggcatcgacggtgacaatgggccccctggaaaagctggccctccgggacccaagggcgagcctggcaaagctgggccagatgggccagacgggaagcccgggattgatggtttaactggagccaagggggagcctggccccatggggatccctggagtcaagggccagcccgggcttcctggtcctcctggccttccgggccctggttttgctggacctcctgggcctcctggacctgttggcctccctggtgagattggaatccgaggccccaagggggaccctggaccagatggaccatcggggcccccaggaccccctgggaaacctggtcgcccgggaaccatccagggtctggaaggcagtgcggatttcctgtgtccaaccaactgtccacccggaatgaaaggtcccccagggctgcagggagtgaaggggcatgcgggcaaacgcgggattctgggtgatcctggccaccaggggaagccgggtcccaagggagatgtgggtgcctctggagagcaaggcatccctggaccaccgggtccccagggcatcaggggctacccaggcatggcagggcccaagggagagacgggccctcatggatataaaggcatggtgggcgctatcggtgccactgggccaccgggtgaggaaggtcctaggggaccgccaggccgagctggggagaagggtgacgagggcagcccaggtattcgtggaccccaggggatcacaggcccgaaaggagcaacgggccccccaggcatcaacggcaaggatgggaccccaggcacgcctggcatgaagggcagtgcaggacaggcgggacagcccggaagtccaggccaccagggcctagcgggtgtgccaggccagcctgggacaaaaggaggccctggagaccagggtgagccgggcccgcagggccttcctggattctctggtccccctgggaaagagggagagccagggcctcgaggagaaattggtccccagggcatcatgggacagaagggtgaccaaggcgagaggggtccagtggggcaaccaggccctcagggaaggcagggccctaagggggagcagggcccccccggaattccagggccccaaggcttgccaggcgtcaaaggagacaagggctccccagggaagaccgggccccgcggcaaagtgggtgacccaggggtggccggcctccccggagagaaaggcgagaagggcgagtccggcgagccggggcccaagggacagcaaggagtacgtggagaacccggctaccctggccccagcggggatgcgggcgccccaggggttcagggctaccctggtccccccggccctcgaggactggccgggaaccgaggcgtgccaggacagcccgggagacagggcgtggagggccgggatgccactgaccagcacatcgtggatgtggcgctgaagatgctgcaagagcaactggcagaggtcgccgtgagtgccaagcgggaagccctgggtgcggtgggcatgatgggtcctccaggacctcctgggccccctgggtacccaggcaagcagggcccccatgggcaccctggccctcggggcgttcctggcatcgtgggagccgtgggtcagatcggcaacacggggcccaagggaaaacgtggagagaagggtgatccaggagaagtgggacgggggcaccccgggatgcctgggcccccagggatcccaggactccctggccggcctggccaggcaatcaacggcaaggatggagatcgagggtccccaggggctccaggagaggcaggtcgacctggcctgccaggccccgtggggctgccgggcttctgtgaacctgccgcctgccttggagcttcggcctatgcctctgcccgccttacagagcctggatccatcaaggggccttgagcatcaggcccagacagagcctggcaggcatcctggcgggaaggaccaggtcccctctgggtggacatgcacccatccccagtccaggaaaccatctcccccaggaccttctgtctgggactcaggagtcctaaggaaaaggaattctaaaacatgggggaaggggaggtagagcactgatgggtgaaaaagtgaggccaacacacagggcaagtggtgtcgatggagtcgaagcgctgaaggaatagggcggctttccttccagcgagcatcattcggctgttaccaaaacaaacatcttaatctgcacctttctccactggccatcttgtccttgggtcagtgggacatgggcacctcgggaggcccgggccctgcccagctacagttccacccctcagcttgaggaccaatgactgaggtctatgccagttcctgatcccatctcactctctggacctaccaggtgactgctgctgggtgactcccctgaggcggctatacccttaagccagccccactacttccttccctgcctcccagctcagtatttaaacatcatctcccttctctttctcgcataactccccacccctttctccccgatccacccaggcctttctgtaaataaaagctcccaagttgggtacaaaccaggatattggagttactctatcctggagttaactagga

SEQ ID NO: 23 - DNA, 2152 bp, Homo sapiens
  source 1..2152: mol_type = genomic DNA
gcaccttccttctgtcaatagatgaaacaaatacttcatcctgctctggaaaccactgcaatgacattattcccagtgctgttgttcctggttgctgggctgcttccatcttttccagcaaatgaagataaggatcccgcttttactgctttgttaaccacccaaacacaagtgcaaagggagattgtgaataagcacaatgaactgaggagagcagtatctccccctgccagaaacatgctgaagatggaatggaacaaagaggctgcagcaaatgcccaaaagtgggcaaaccagtgcaattacagacacagtaacccaaaggatcgaatgacaagtctaaaatgtggtgagaatctctacatgtcaagtgcctccagctcatggtcacaagcaatccaaagctggtttgatgagtacaatgattttgactttggtgtagggccaaagactcccaacgcagtggttggacattatacacaggttgtttggtactcttcatacctcgttggatgtggaaatgcctactgtcccaatcaaaaagttctaaaatactactatgtttgccaatattgtcctgctggtaattgggctaatagactatatgtcccttatgaacaaggagcaccttgtgccagttgcccagataactgtgacgatggactatgcaccaatggttgcaagtacgaagatctctatagtaactgtaaaagtttgaagctcacattaacctgtaaacatcagttggtcagggacagttgcaaggcctcctgcaattgttcaaacagcatttattaaatacgcattacacaccgagtagggctatgtagagaggagtcagattatctacttagatttggcatctacttagatttaacatatactagctgagaaattgtaggcatgtttgatacacatttgatttcaaatgtttttcttctggatctgctttttattttacaaaaatatttttcatacaaatggttaaaaagaaacaaaatctataacaacaactttggatttttatatataaactttgtgatttaaatttactgaatttaattagggtgaaaattttgaaagttgtattctcatatgactaagttcactaaaaccctggattgaaagtgaaaattatgttcctagaacaaaatgtacaaaaagaacaatataattttcacatgaacccttggctgtagttgcctttcctagctccactctaaggctaagcatcttcaaagacgttttcccatatgctgtcttaattcttttcactcattcacccttcttcccaatcatctggctggcatcctcacaattgagttgaagctgttcctcctaaaacaatcctgacttttattttgccaaaatcaatacaatcctttgaattttttatctgcataaattttacagtagaatatgatcaaaccttcatttttaaacctctcttctctttgacaaaacttccttaaaaaagaatacaagataatataggtaaataccctccactcaaggaggtagaactcagtcctctcccttgtgagtcttcactaaaatcagtgactcacttccaaagagtggagtatggaaagggaaacatagtaactttacaggggagaaaaatgacaaatgacgtcttcaccaagtgatcaaaattaacgtcaccagtgataagtcattcagatttgttctagataatctttctaaaaattcataatcccaatctaattatgagctaaaacatccagcaaactcaagttgaaggacattctacaaaatatccctggggtattttagagtattcctcaaaactgtaaaaatcatggaaaataagggaatcctgagaaacaatcacagaccacatgagactaaggagacatgtgagccaaatgcaatgtgcttcttggatcagatcctggaacagaaaaagatcagtaatgaaaaaactgatgaagtctgaatagaatctggagtatttttaacagtagtgttgatttcttaatcttgataaatatagcagggtaatgtaagatgataacgttagagaaactgaaactgggtgagggctatctaggaattctctgtactatcttaccaaattttcggtaagtctaagaaagcaatgcaaaataaaaagtgtcttgaaaaaaaa

SEQ ID NO: 24 - DNA, 746 bp, Homo sapiens
  source 1..746: mol_type = genomic DNA
gatccccgcctcaggctctcaacctcctctcctgcagctccagctctgtgctctgcctccgaggagaccatggcctggcccctgtgcaccctgctgctcctgctggccacccaggctgtggccctggcctggagcccccaggaggaggacaggataatcgagggtggcatctatgatgcagacctcaatgatgagcgggtacagcgtgcccttcactttgtcatcagcgagtataacaaggccactgaagatgagtactacagacgcctgctgcgggtgctacgagccagggagcagatcgtgggcggggtgaattacttcttcgacatagaggtgggccgaaccatatgtaccaagtcccagcccaacttggacacctgtgccttccatgaacagccagaactgcagaagaaacagttgtgctctttccagatctacgaagttccctgggaggacagaatgtccctggtgaattccaggtgtcaagaagcctagggatctgtgccagggagtcacactgaccacctcctactcccaccccttgtagtgctcccacccctggactggtggcccccaccctgtgggaggtctccccatgcacctgcagcaggagaagacagagaaggctgcaggaggcctttgttgctcagcaggggactctgccctccctccttccttttgcttctcatagccctggtacatggtacacacacccccacctcctgcaattaaacagtagcatcacc

SEQ ID NO: 25 - DNA, 2356 bp, Homo sapiens
  source 1..2356: mol_type = genomic DNA
agcagcatcatgcttagacttttcaaagagacaaactccattttcttatgaatggaaagtgaaaacccctgttccgcttaaattgggttccttcctgtcctgagaaacatagagacccccaaaagggaagcagaggagagaaagtcccacacccagaccccgcgagaagagatgaccatgaccaccatgccagaaagtctcaacagccccgtgtcgggcaaggcggtgtttatggagtttgggccgcccaaccagcaaatgtctccttctcccatgtcccacgggcactactccatgcactgtttacactcggcgggccattcgcagcccgacggcgcctacagctcagcctcgtccttctcccgaccgctgggctacccctacgtcaactcggtcagcagccacgcatccagcccctacatcagttcggtgcagtcctacccgggcagcgccagcctcgcccagagccgcctggaggacccaggggcggactcggagaagagcacggtggtggaaggcggtgaagtgcgcttcaatggcaagggaaaaaagatccgtaaacccaggacgatttattccagtttgcagttgcaggctttgaaccggaggttccagcaaactcagtacctagctctgccggagagggcggagctcgcggcctctttgggactcacacagactcaggtcaagatctggttccaaaacaagcgatccaagttcaagaagctgatgaagcagggtggggcggctctggagggtagtgcgttggccaacggtcgggccctgtctgctggctccccacccgtgccgcccggctggaaccctaactcttcatccgggaagggctcaggaggaaacgcgggctcctatatccccagctacacatcgtggtacccttcagcgcaccaagaagctatgcagcaaccccaacttatgtgaggttgcccgcccgtctccttcttgtctccccggcccaggtccctcccgcctccaggtccatccatcccgtccggaaaagaaggacccagagggaagaaggaacagtggaggcgggacgccctccatctcctcggagccccgcgaggtccggcccagcaacttcccggcatccgcgctctagcctgaaccctggcctgggccgagcagtggcagcagagagtggcctcggagggaagccactgccacctgagacagcccaagcagcaagataaacccgctccacccgacccgccgaccttcagctttgtgggactatcaggaaaaaacaaaacaaaaacaaaatgtagaaaaagcaaaagctcttttctgtcctgtcagtctcctgtctccttttgctctgtctgtgcgctggtaaagtccaggtcctcatccgtccgctgtcctcattctgcggcctcagcaaaaagccacaaggtctgagcggcccgggtcctgccgggctgaccatctccggatcctgggacactctgcctgaccatctgtgtagctggtgtgggaatctgggggcattggagggagggggttttatttattgagaaatggacttcgcctgaggctgtttgccaattcagggttctgctgggcgcaaggaacgcactgttcaaacgcactgtttactttaagcgcacggggagaaacgaataaggaggacgtggtgatttttaatttatacagtaacttttgtacttctctggtatggagagtttggagccgaatgatttgcattttttacatgtccgacattatttaataaataatttttaaaagaaaagaacgataaatgaagccaacatgattttctcatttcgggaggaactctgttgcttcgcctggacaagaaggaaaatgctgatttcctccttgggtagaaagagggagcgagggcaaatggggagtagagagaaaacaggcgagaacaagcactctaattccagtgggctttaaaataagacaaaatcagctttacaacaatccctagaggctcgaccacagaataatgccagtcaccaccctgaacgcacaatctccagtgcaggatctaatgactgtacatattattgttattattattattgttattattgttgttctgtaaacatgttgcacaagcttagcctttttgcgttctgttgtgtgtggctgtaaaaccccatgctttgtgaaatgagaatcttgacatttttcttgtgaaatttggaaaatgtgatcaattgaaatcaactgtgttttgtgttctctatgtcaaagtttagttttatattgagaatgttaacttattgctttgtatcttgggaaaaaaactttgtaaataagttataaagtttctttgagacagtaaaattatgatttcttgaaa

SEQ ID NO: 26 - DNA, 6193 bp, Homo sapiens
  source 1..6193: mol_type = genomic DNA
agaggcgctttcggcttccaagggggaagtgctgggctataattaatgtttttattaaatttggagggaagtttttgcagcctttcgcctagcgtggccttcaggttgatagaagtccagatcctgaggaaatctccagctaaatgctcaaaatataaaatactgagctgagatttgcgaagagcagcagcatggatggattttatgaccagcaagtgccttacatggtcaccaatagtcagcgtgggagaaattgtaacgagaaaccaacaaatgtcaggaaaagaaaattcattaacagagatctggctcatgattcagaagaactctttcaagatctaagtcaattacaggaaacatggcttgcagaagctcaggtacctgacaatgatgagcagtttgtaccagactatcaggctgaaagtttggcttttcatggcctgccactgaaaatcaagaaagaaccccacagtccatgttcagaaatcagctctgcctgcagtcaagaacagccctttaaattcagctatggagaaaagtgcctgtacaatgtcagtgcctatgatcagaagccacaagtgggaatgaggccctccaacccccccacaccatccagcacgccagtgtccccactgcatcatgcatctccaaactcaactcatacaccgaaacctgaccgggccttcccagctcacctccctccatcgcagtccataccagatagcagctaccccatggaccacagatttcgccgccagctttctgaaccctgtaactcctttcctcctttgccgacgatgccaagggaaggacgtcctatgtaccaacgccagatgtctgagccaaacatccccttcccaccacaaggctttaagcaggagtaccacgacccagtgtatgaacacaacaccatggttggcagtgcggccagccaaagctttccccctcctctgatgattaaacaggaacccagagattttgcatatgactcaggctgtatgtttgaaaagggccccaggcagttttatgatgacacctgtgttgtcccagaaaaattcgatggagacatcaaacaagagccaggaatgtatcgggaaggacccacataccaacggcgaggatcacttcagctctggcagtttttggtagctcttctggatgacccttcaaattctcattttattgcctggactggtcgaggcatggaatttaaactgattgagcctgaagaggtggcccgacgttggggcattcagaaaaacaggccagctatgaactatgataaacttagccgttcactccgctattactatgagaaaggaattatgcaaaaggtggctggagagagatatgtctacaagtttgtgtgtgatccagaagcccttttctccatggcctttccagataatcagcgtccactgctgaagacagacatggaacgtcacatcaacgaggaggacacagtgcctctttctcactttgatgagagcatggcctacatgccggaagggggctgctgcaacccccacccctacaacgaaggctacgtgtattaacacaagtgacagtcaagcagggcgtttttgcgcttttccttttttctgcaagatacagagaattgctgaatctttgttttatttctgttgtttgtattttatttttaaataataatacacaaaaaggggcttttcctgttgcattattctatggtctgccatggactgtgcactttatttgagggtgggtgggagtaatctaaacatttattctgtgtaacaggaagctaatgggtgaatgggcagagggatttggggattactttttacttaggcttgggatggggtcctacaagttttgagtatgatgaaactatatcatgtctgtttgatttcataacaacataagataatgtttattttatcggggtatctatggtacagttaatttcacgttgtgtaaatatccacttggagactatttgccttgggcattttcccctgtcatttatgagtctctgcaggtgtacaaaaaaaccccaatctactgtaaatggcagtttaattgttagaaatgactgtttttgcaccacttgtaaaaaggtatttagcgattgcatttgctgtttgttgttttattttgctttatatatgacttgcagaggataaccataaaatgggtaattctctctgaagttgaataatcaccatgactgtaaatgaggggcacaattttggactctggcgccaaactgagtcataggccagtagcattacgtgtatctggtgccaccttgctgtttagatacaaatcataccgtcttttaaatattttgaagcccatttcagttaaataatgacatgtcatggtcctttggaatcttcatttaaatgttaaatctggaatcaaaatgaagcaaaaaatatctgtctccttttcactttcttcagtacataaatacattatttaatcaataagaattaactgtactaaatcatgtattatgctgttctagttacagcaaacactctttaagaaaaatatccaatacactaaataggtactatagtaatttttagacatggtacccattgatatgcatttaaaccttttactgctgtgttatgttgataacatatataaatattagataatgctaatgcttctgctgctgtcttttctgtaatattctctttcatgctgaatttactatgaccatttataagcagtgcagttaactacagatagcatttcaggacaaaatagatgactcaaaccatttattgcttaaaaaatagcttacgccatgctatgctataagcagcttttatgcacattgacaaatgaagagtaagcttcagcttgctaaaggaaactgtggaaccttttgtaacttttggtgatatggaaaattatttacaaaccgtcaaagaatatgaggaagttgctgtatgacatagtgctggcactgatattatccatcatctctttttggacacttctgtaaatgtgattggattgtttgaaagaagatttaaagtttcaaagttttttgttctgtttttgctttgcatttggagaaaatattgaaagcagggtatgttgtttcattcaccttgaaaaaaccatgagtaaatggggatatagaatctctgaatagctcgctaaaagattcaagcaagggacatgaattttgttccatctatcaataatatccagaagaacaacttttttaaagagtctatagcaaaaagcaaaaaaaaaaaaaaattctaaacacaaagtcaaaataaacctattgtaaaagcatttcgtgatgagcatgaaaaagattgtttaaagatgatccccccagctacccattttccaaaactacacagatcacagctcatttctctaagtggagcagttatcaagaaacccaaacaccaaaattgctactcttcacatttaatcctacaaaaagtactccaatttcaaaatatgtatgtaacctgcgatttcaatgattgttgttcatatacatcatgtattattttggcccattttgggcctaaaaaagaaaactatgccttaaaaatcagaaccttttctccccactatgcttatgtggccatctacagcacttagaataaaaacagatgttaaaatattcagtgaaagttttattggaaaaaggaattgagatatataattgagatttggtgaaattgaaggagaaaatttaagtgagtctttaaaatatattctgaatgaaaactgtattgaggattcatttttgttccttttttttctttttctcttttctcctttttcttctttttaatagtctagttttagtcagtcagtgaggaagaattgggccatgctaacgttatcacaagagaacaatggcagaaatggtattagttatataatatttaaggacaaactatatgttttgctgttttaacgtagtgactcactgaactaaatacataattgaccaacattaagtgtatttccaatacagaagggttgaaaatattacattataaactcttttgaaaaatgtatctaaaattttttaagttctgttttgattccactttttggttgagtttttatgtttttgttttcaggtagattaataaatctggcagctgatttctgcaagattcttgtgttttgaatttctcattgaattggctactcaaacatagaaatcatttgttaatgatgtaatgtcttctctcagcttttatcttcactgctgtttgctgtctcttgatgatgacatgttaatacccaatagattaattgcaacaaacacttatactcaaataactaagtaaaaataatttttcttgttatgtccatgaaaagtgcttcagaataaaaatccacaagactgacagtgcagaacatttttctcaaatcatgggcggatcttggaggtctagtttcccgtagatgctgtaaccaattaccacaacttcagtaatttacacaaatttatcttatagttctggaggcagaagttcaaaagaagccttaagagactaaaaccaagatgtccttaggtctggttccttctggaggctccaggggagattcttccagctttcacttctagagtctgctgacattccttggctcctggctacatcacttcaatctctgcttccatggtcacatactcttctactatagtcaaatttccttcctgcctcttataaggatgcttgtgattacatttaggggatgctcagataatccaggacaatctctccatctcaagatccttaacttaatgacgtgtgccaagtccctttggctagataattattcataggtcccagggattaggacatggatgtaaggggtgagggcagggctgttattcagaacaccgcacggaggaggaagactgtgtagcaaagactctaattgatttactcaggaacagtggagttctgctgagggatctaggatttgaaagtactagagtttgcttttatttaccactgagatattttccccttattctgcataaataattttgaaaactttctatattaaatttcaactattccactaaaatgtctggtaatcacatcaagcctttagattattcaaatccttccccagcccccaggaaaacactaagtcatgaaacagaaaaacagaaggtatgataataatagtaataacagttaaatcagtggtctaatccagattttattttttaatacatttcttttggtgttaatatgggttactatgtgatcttatcatttgctagtgattattacttattaggtaagaacaatgtgtaaaatatgtctattactcaaaagaacaattgcaaaatgagtcaacttatctttatataaccaggaaagaaatatattgccagaagctacagaattttgccagatgatagggatttctaaaatgagccactttgtctatcatgcagccttttcagagcttgtaatgagaaaacattacagaggagaaggtcatttggatgtttgttacttggaatcctagaaaacaaaaactaaaatttaaaaataagaagtgagtaagctattttccatttgcgatttggtatggagaagagaggaaatagaattattaaaaaaatacaaattgggtaaaagtgatggtggaaaaaatataaagaaggcaaatgtacatattaagcaattctactaagaattggaaaaatcaagtttcaaaaagatggtaatagttgggcatgatactagaaaatttcacccagtttattcagagctcaactagtacttttaggacttctttttttatatacatgagactcactttgacatacttaaaaaaaaaacagtttatggaaagtacagtttaagaggagaatttgattagactaagtggatatctttatagaaatattaatgatttcagaattttcagttacaagtgtatataccgtggctattgtttatggattcatatgtaaggtagggtcttttttgcatatagactccagtattagttactttcattctaaaattatatttatgcttctatggggaagaaaatttttaattcacttggttgtattaaaattatacttacggtttgagaaaacatgctatgaaaatcatgattatagcaaattaaatatgctcaaaatttaaatctaaaataaaagcccagaaactgaaaa

SEQ ID NO: 27 - DNA, 9132 bp, Homo sapiens
  source 1..9132: mol_type = genomic DNA
attgcagctgggacagcccggagtgtggttagcagctcggcaagcgctgcccaggtcctggggtggtggcagccagcgggagcaggaaaggaagcatgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcggagaagatggacgacgctgtggctccaggccgagaatacacctatgaatggagtatcagtgaggacagtggacccacccatgatgaccctccatgcctcacacacatctattactcccatgaaaatctgatcgaggatttcaactcggggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcgaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaatcaggtcattccgaaactcatcattgaatcaggaagaagaagagttcaatcttactgccctagctctggagaatggcactgaattcgtttcttcaaacacagatataattgttggttcaaattattcttccccaagtaatattagtaagttcactgtcaataaccttgcagaacctcagaaagccccttctcaccaacaagccaccacagctggttccccactgagacacctcattggcaagaactcagttctcaattcttccacagcagagcattccagcccatattctgaagaccctatagaggatcctctacagccagatgtcacagggatacgtctactttcacttggtgctggagaattcaaaagtcaagaacatgctaagcataagggacccaaggtagaaagagatcaagcagcaaagcacaggttctcctggatgaaattactagcacataaagttgggagacacctaagccaagacactggttctccttccggaatgaggccctgggaggaccttcctagccaagacactggttctccttccagaatgaggccctggaaggaccctcctagtgatctgttactcttaaaacaaagtaactcatctaagattttggttgggagatggcatttggcttctgagaaaggtagctatgaaataatccaagatactgatgaagacacagctgttaacaattggctgatcagcccccagaatgcctcacgtgcttggggagaaagcacccctcttgccaacaagcctggaaagcagagtggccacccaaagtttcctagagttagacataaatctctacaagtaagacaggatggaggaaagagtagactgaagaaaagccagtttctcattaagacacgaaaaaagaaaaaagagaagcacacacaccatgctcctttatctccgaggacctttcaccctctaagaagtgaagcctacaacacattttcagaaagaagacttaagcattcgttggtgcttcataaatccaatgaaacatctcttcccacagacctcaatcagacattgccctctatggattttggctggatagcctcacttcctgaccataatcagaattcctcaaatgacactggtcaggcaagctgtcctccaggtctttatcagacagtgcccccagaggaacactatcaaacattccccattcaagaccctgatcaaatgcactctacttcagaccccagtcacagatcctcttctccagagctcagtgaaatgcttgagtatgaccgaagtcacaagtccttccccacagatataagtcaaatgtccccttcctcagaacatgaagtctggcagacagtcatctctccagacctcagccaggtgaccctctctccagaactcagccagacaaacctctctccagacctcagccacacgactctctctccagaactcattcagagaaacctttccccagccctcggtcagatgcccatttctccagacctcagccatacaaccctttctccagacctcagccatacaaccctttctttagacctcagccagacaaacctctctccagaactcagtcagacaaacctttctccagccctcggtcagatgcccctttctccagacctcagccatacaaccctttctctagacttcagccagacaaacctctctccagaactcagccatatgactctctctccagaactcagtcagacaaacctttccccagccctcggtcagatgcccatttctccagacctcagccatacaaccctttctctagacttcagccagacaaacctctctccagaactcagtcaaacaaacctttccccagccctcggtcagatgcccctttctccagaccccagccatacaaccctttctctagacctcagccagacaaacctctctccagaactcagtcagacaaacctttccccagacctcagtgagatgcccctctttgcagatctcagtcaaattccccttaccccagacctcgaccagatgacactttctccagaccttggtgagacagatctttccccaaactttggtcagatgtccctttccccagacctcagccaggtgactctctctccagacatcagtgacaccacccttctcccggatctcagccagatatcacctcctccagaccttgatcagatattctacccttctgaatctagtcagtcattgcttcttcaagaatttaatgagtcttttccttatccagaccttggtcagatgccatctccttcatctcctactctcaatgatacttttctatcaaaggaatttaatccactggttatagtgggcctcagtaaagatggtacagattacattgagatcattccaaaggaagaggtccagagcagtgaagatgactatgctgaaattgattatgtgccctatgatgacccctacaaaactgatgttaggacaaacatcaactcctccagagatcctgacaacattgcagcatggtacctccgcagcaacaatggaaacagaagaaattattacattgctgctgaagaaatatcctgggattattcagaatttgtacaaagggaaacagatattgaagactctgatgatattccagaagataccacatataagaaagtagtttttcgaaagtacctcgacagcacttttaccaaacgtgatcctcgaggggagtatgaagagcatctcggaattcttggtcctattatcagagctgaagtggatgatgttatccaagttcgttttaaaaatttagcatccagaccgtattctctacatgcccatggactttcctatgaaaaatcatcagagggaaagacttatgaagatgactctcctgaatggtttaaggaagataatgctgttcagccaaatagcagttatacctacgtatggcatgccactgagcgatcagggccagaaagtcctggctctgcctgtcgggcttgggcctactactcagctgtgaacccagaaaaagatattcactcaggcttgataggtcccctcctaatctgccaaaaaggaatactacataaggacagcaacatgcctatggacatgagagaatttgtcttactatttatgacctttgatgaaaagaagagctggtactatgaaaagaagtcccgaagttcttggagactcacatcctcagaaatgaaaaaatcccatgagtttcacgccattaatgggatgatctacagcttgcctggcctgaaaatgtatgagcaagagtgggtgaggttacacctgctgaacataggcggctcccaagacattcacgtggttcactttcacggccagaccttgctggaaaatggcaataaacagcaccagttaggggtctggccccttctgcctggttcatttaaaactcttgaaatgaaggcatcaaaacctggctggtggctcctaaacacagaggttggagaaaaccagagagcagggatgcaaacgccatttcttatcatggacagagactgtaggatgccaatgggactaagcactggtatcatatctgattcacagatcaaggcttcagagtttctgggttactgggagcccagattagcaagattaaacaatggtggatcttataatgcttggagtgtagaaaaacttgcagcagaatttgcctctaaaccttggatccaggtggacatgcaaaaggaagtcataatcacagggatccagacccaaggtgccaaacactacctgaagtcctgctataccacagagttctatgtagcttacagttccaaccagatcaactggcagatcttcaaagggaacagcacaaggaatgtgatgtattttaatggcaattcagatgcctctacaataaaagagaatcagtttgacccacctattgtggctagatatattaggatctctccaactcgagcctataacagacctacccttcgattggaactgcaaggttgtgaggtaaatggatgttccacacccctgggtatggaaaatggaaagatagaaaacaagcaaatcacagcttcttcgtttaagaaatcttggtggggagattactgggaacccttccgtgcccgtctgaatgcccagggacgtgtgaatgcctggcaagccaaggcaaacaacaataagcagtggctagaaattgatctactcaagatcaagaagataacggcaattataacacagggctgcaagtctctgtcctctgaaatgtatgtaaagagctataccatccactacagtgagcagggagtggaatggaaaccatacaggctgaaatcctccatggtggacaagatttttgaaggaaatactaataccaaaggacatgtgaagaactttttcaaccccccaatcatttccaggtttatccgtgtcattcctaaaacatggaatcaaagtattgcacttcgcctggaactctttggctgtgatatttactagaattgaacattcaaaaacccctggaagagactctttaagacctcaaaccatttagaatgggcaatgtattttacgctgtgttaaatgttaacagttttccactatttctctttcttttctattagtgaataaaattttatacaagaagcttttataatgtaactccttgctaccagtaagtaagataatggctattacttctgcattaatttgaatacaggtaggaaaatatcaagaaccaacaagaaaagggcttatctttcttaatgattgaaaatgctatgaagtaatatttatgtagttaaaatgcttcattataactcttttaaatcctttacacactagtaaaacagatattactttaaataataattgatagacctggataactttcacaaacacatgattttttaatggtttttcttgagtgaagagaaaaacaatattatcaaatgaaataagtacttaaaatatcctgtctttcccatataacaatgatttttctgactttccatgagtaaaaaaacagccaagcatctttccagtagccccattgaaattgtgaatccgtcctggtctccctaaggactgcacacattgatattcaaggttggtggtcattagatatggaacagaactgaaataaccatggtagaactgaatgtgtaatgttggctttattctagctggtactacatggcacacagtttcaaaacataatttcacctactggaaagctcagacctgtaaaacagagcatgggaactgctggtctaaatgcagttgttcctgctcaaagagacctctggccaaactggcaagcagttaaagttttctttcagggccttcctctctatggcctcaacttcctcctctctcttcttccagcaacttcccctttcatcattcctttccctggggacttggcattcagtgatcctgtagatattgcacaactggggaacctttagacatccttaaaatcacatgagatagacagtcatttggggtgtctgaaataaaccaccccaaaacttagtgttaaaagagcaaccaaaaaaaatttatgtgagattatggatttgttacttagcttgatttaatcatcctgtaacgtgtacatatatcaaaatgttatgtataccataaatatataaaattttatcaacgaaattcataacaatctctcagaccacagagaaatcaaattagaactgaggactaagaaactcactcgaaaccacacaactacatggaaactgaacaacctgctcctgaatgactactgggtaaataatgaaattaaggcagaaataaataagttccttaaaaccaatgagaacaaagagacaacataccagaatctctaggagacagggctttgcttttgctgcattctattcgttgtgaacacaaattacaggccagtctcgattcagtgtagaagggaactgcataaggaccacataccaggaggcataattcactgggagcatctttagaaactaccagagttacctgttgcccataccagtggggtaagccctatgaatgtatatgagagtttcaaacatccacaaaacattggctttctaatattcgtattcccactattcctttcttttcatgattcatgtcattgtcccatcaacatttctaagatttccattccgttaagagcaaaagagaatgttggaaggtgggggaaaacatttctttgttttctacagggccagcttcttggatgtgtgtgatctgttcagttgcaaagggtcacatgctcagaaggaccgcatgctaaatttaatgctttgcagttaccctcttgaaatcctttattttttaagaaggaattcgacatttccatttttcaatgagccccacaaattacgcagctagtcctgggcttctctactctgaaattgggcaggatctctcttgatctagaatttactaaggcataataggggcaagaaaatcttatgaaataatggggggtagggaagagatgggaatggagcatgagatccagcttcgttattctctacttgagaaaaataaggccccaaagattaaacaacttgcccaaggatattgcttgttagtgtcagaactgaaaccagaaaccaaatgatcatatccctagacttttagtctgctttctcttccataaaatgaaacttataatgtttctaatccattgctcagacaggtagacatgaatattaattgataatgactattaattgatctggaaaatacttgtttggggatcaataatatgtttgggctattatctaatgctgtgtagaaatattaaaacccctgttattttgaaataaaaaagatacccactttttat

SEQ ID NO: 28 - DNA, 1200 bp, Homo sapiens
  source 1..1200: mol_type = genomic DNA
agtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcggaggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgtt

SEQ ID NO: 29 - DNA, 1617 bp, Homo sapiens
  source 1..1617: mol_type = genomic DNA
cagttgccatgggccaggagtctcacagaactactaagcagctgcttacccaattttggctggagagataagtaccctctggggccaccacagctggccccaaccatggtcctgattgccttggaagaactgcctttggaatttcacatcggcatccagatagatggtgtcacaagaaggatctgaagtggagcttctagtatccccaggagcgcgaagtgaacacggaaggtacctgcaggatccaattgtgtccattgatctctcagagtggctgaggataatagagtttcttcttcaaggtctcaaggtctgaagcatcccacagaatgatcctactgaataactcccataagctgctggccctatacaaatccttggccaggagcatccctgagtccctgaaggtgtatggctctgtgtatcacatcaatcacgggaaccccttcaacatggaggtgctggtggattcctggcctgaatatcagatggttattatccggcctcaaaagcaggagatgactgatgacatggattcatacacaaacgtatatcgtatgttctccaaagagcctcaaaaatcagaagaagttttgaaaaattgtgagatcgtaaactggaaacagagactccaaatccaaggtcttcaagaaagtttaggtgaggggataagagtggctacattttcaaagtcagtgaaagtagagcattcgagagcactcctcttggttacggaagatattctgaagctcaatgcctccagtaaaagcaagcttggaagctgggctgagacaggccacccagatgatgaatttgaaagtgaaactcccaactttaagtatgcccagctggatgtctcttattctgggctggtaaatgacaactggaagcgagggaagaatgagaggagcctgcattacatcaagcgctgcatagaagacctgccagcagcctgtatgctcggcccagagggagtcccggtctcatgggtaaccatggacccttcttgtgaagtaggaatggcctacagcatggaaaaataccgaaggacaggcaacatggcacgagtgatggtgcgatacatgaaatatctgcgtcagaagaatattccattttacatctctgtgttggaagaaaatgaagactcccgcagatttgtggggcagtttggtttctttgaggcctcctgtgagtggcaccaatggacttgctacccacagaatctagttccattttagacaatgaagctgcttagtaatctctgccaagccatctcttaatattaaagcagacaccacagaatagatttcttcacttacaaatgcatattgggcacttataatacagcaggaactcttctcacctggagccttgatgttaaaagacacagccatgctcttgaggagcttacaatcctggctggaggcaggggagggtatattctttaaatatgcttaagtgttatagggaaagacggggttaccagtaaacatgtaactagaaagccaggctcagttcttacctctgggaatcagaactctttatgcaacttggttaatagaatctactatctggaagataaatgaaggattttaataaaattttcaatagaataaa

SEQ ID NO: 30 - DNA, 3023 bp, Homo sapiens
  source 1..3023: mol_type = genomic DNA
atcttcactttttccgttgctagcagtggaagggtcacagaccaaacactaaggcctgagcggtgacaaccgaggcgagatgatggtcaacagggaatgcctcgtgggagaaaaaagacaattttattctcagcgctgattttgagatgatgggcttgggaaacgggcgtcgcagcatgaagtcgccgcccctcgtgctggccgccctggtggcctgcatcatcgtcttgggcttcaactactggattgcgagctcccggagcgtggacctccagacacggatcatggagctggaaggcagggtccgcagggcggctgcagagagaggcgccgtggagctgaagaagaacgagttccagggagagctggagaagcagcgggagcagcttgacaaaatccagtccagccacaacttccagctggagagcgtcaacaagctgtaccaggacgaaaaggcggttttggtgaataacatcaccacaggtgagaggctcatccgagtgctgcaagaccagttaaagaccctgcagaggaattacggcaggctgcagcaggatgtcctccagtttcagaagaaccagaccaacctggagaggaagttctcctacgacctgagccagtgcatcaatcagatgaaggaggtgaaggaacagtgtgaggagcgaatagaagaggtcaccaaaaaggggaatgaagctgtagcttccagagacctgagtgaaaacaacgaccagagacagcagctccaagccctcagtgagcctcagcccaggctgcaggcagcaggcctgccacacacagaggtgccacaagggaagggaaacgtgcttggtaacagcaagtcccagacaccagcccccagttccgaagtggttttggattcaaagagacaagttgagaaagaggaaaccaatgagatccaggtggtgaatgaggagcctcagagggacaggctgccgcaggagccaggccgggagcaggtggtggaagacagacctgtaggtggaagaggcttcgggggagccggagaactgggccagaccccacaggtgcaggctgccctgtcagtgagccaggaaaatccagagatggagggccctgagcgagaccagcttgtcatccccgacggacaggaggaggagcaggaagctgccggggaagggagaaaccagcagaaactgagaggagaagatgactacaacatggatgaaaatgaagcagaatctgagacagacaagcaagcagccctggcagggaatgacagaaacatagatgtttttaatgttgaagatcagaaaagagacaccataaatttacttgatcagcgtgaaaagcggaatcatacactctgaattgaactggaatcacatatttcacaacagggccgaagagatgactataaaatgttcatgagggactgaatactgaaaactgtgaaatgtactaaataaaatgtacatctgaagatgattattgtgaaattttagtatgcactttgtgtaggaaaaaatggaatggtcttttaaacagcttttggggggtactttggaagtgtctaataaggtgtcacaatttttggtagtaggtatttcgtgagaagttcaacaccaaaactggaacatagttctccttcaagtgttggcgacagcggggcttcctgattctggaatataactttgtgtaaattaacagccacctatagaagagtccatctgctgtgaaggagagacagagaactctgggttccgtcgtcctgtccacgtgctgtaccaagtgctggtgccagcctgttacctgttctcactgaaaagtctggctaatgctcttgtgtagtcacttctgattctgacaatcaatcaatcaatggcctagagcactgactgttaacacaaacgtcactagcaaagtagcaacagctttaagtctaaatacaaagctgttctgtgtgagaattttttaaaaggctacttgtataataacccttgtcatttttaatgtacaaaacgctattaagtggcttagaatttgaacatttgtggtctttatttactttgcttcgtgtgtgggcaaagcaacatcttccctaaatatatattaccaagaaaagcaagaagcagattaggtttttgacaaaacaaacaggccaaaagggggctgacctggagcagagcatggtgagaggcaaggcatgagagggcaagtttgttgtggacagatctgtgcctactttattactggagtaaaagaaaacaaagttcattgatgtcgaaggatatatacagtgttagaaattaggactgtttagaaaaacaggaatacaatggttgtttttatcatagtgtacacatttagcttgtggtaaatgactcacaaaactgattttaaaatcaagttaatgtgaattttgaaaattactacttaatcctaattcacaataacaatggcattaaggtttgacttgagttggttcttagtattatttatggtaaataggctcttaccacttgcaaataactggccacatcattaatgactgacttcccagtaaggctctctaaggggtaagtaggaggatccacaggatttgagatgctaaggccccagagatcgtttgatccaaccctcttattttcagaggggaaaatggggcctagaagttacagagcatctagctggtgcgctggcacccctggcctcacacagactcccgagtagctgggactacaggcacacagtcactgaagcaggccctgtttgcaattcacgttgccacctccaacttaaacattcttcatatgtgatgtccttagtcactaaggttaaactttcccacccagaaaaggcaacttagataaaatcttagagtactttcatactcttctaagtcctcttccagcctcactttgagtcctccttggggttgataggaattttctcttgctttctcaataaagtctctattcatctcatgtttaatttgtacgcatagaattgctgagaaataaaatgttctgttcaactta

SEQ ID NO: 31 - DNA, 7838 bp, Homo sapiens
  source 1..7838: mol_type = genomic DNA
agtcgcgttgctcctccgaggaagcaagcggcggtggcgactcggtggaaaaataacgaaagaaaggcagagaggaagtagcgagagaagagagaaaatgaagtcggcgctgggggagcctgcaggagggtggccaacagtggaggaaggtggatttggcttcttttccgcaccccgggcgtgaaagccctctccaacgcgaccccaggaaataagtgggtctcgcctgggcagaaaaggaaaagaatccaggcgagagcgcgtcgctcctctgtcactgctgcccccgaggaactccggctgcttctcatcccggccgcctcgcggggccggacgcagtgcccgaggcgccctgcagatggggcgggcagggaacgggcgctccagctgcgggtgacaggcgccggcccgcccgcctgcctgctcagcgcagtgaccgggcgggcagaggatgccaggcggagggacctgggagcgggatctgagactgccggaggcgcgctacgctccaacttgcatggcctagagaccgctccagctcctgggaccgcttcaccgagtggagtgaagctgcgcgcgggacctggaggcggagacctcaggcagcggctgcagaggggcgagccgggcgcaggagggggcgcgctttctccctgcgggtctcagtaatgaggagactgagtttgtggtggctgctgagcagggtctgtctgctgttgccgccgccctgcgcactggtgctggccggggtgcccagctcctcctcgcacccgcagccctgccagatcctcaagcgcatcgggcacgcggtgagggtgggcgcggtgcacttgcagccctggaccaccgccccccgcgcggccagccgcgctccggacgacagccgagcaggagcccagagggatgagccggagccagggactaggcggtccccggcgccctcgccgggcgcacgctggttggggagcaccctgcatggccgggggccgccgggctcccgtaagcccggggagggcgccagggcggaggccctgtggccacgggacgccctcctatttgccgtggacaacctgaaccgcgtggaagggctgctaccctacaacctgtctttggaagtagtgatggccatcgaggcaggcctgggcgatctgccacttttgcccttctcctcccctagttcgccatggagcagtgaccctttctccttcctgcaaagtgtgtgccataccgtggtggtgcaaggggtgtcggcgctgctcgccttcccccagagccagggcgaaatgatggagctcgacttggtcagcttagtcctgcacattccagtgatcagcatcgtgcgccacgagtttccacgggagagtcagaatccccttcacctacaactgagtttagaaaattcattaagttctgatgctgatgtcactgtctcaatcctgaccatgaacaactggtacaattttagcttgttgctgtgccaggaagactggaacatcaccgacttcctcctccttacccagaataattccaagttccaccttggttctatcatcaacatcaccgctaacctcccctccacccaggacctcttgagcttcctacagatccagcttgagagtattaagaacagcacacccacagtggtgatgtttggctgcgacatggaaagtatccggcggattttcgaaattacaacccagtttggggtcatgccccctgaacttcgttgggtgctgggagattcccagaatgtggaggaactgaggacagagggtctgcccttagggctcattgctcatggaaaaacaacacagtctgtctttgagcactacgtacaagatgctatggagctggtcgcaagagctgtagccacagccaccatgatccaaccagaacttgctctcattcccagcacgatgaactgcatggaggtggaaactacaaatctcacttcaggacaatatttatcaaggtttctagccaataccactttcagaggcctcagtggttccatcagagtaaaaggttccaccatcgtcagctcagaaaacaactttttcatctggaatcttcaacatgaccccatgggaaagccaatgtggacccgcttgggcagctggcaggggggaaagattgtcatggactatggaatatggccagagcaggcccagagacacaaaacccacttccaacatccaagtaagctacacttgagagtggttaccctgattgagcatccttttgtcttcacaagggaggtagatgatgaaggcttgtgccctgctggccaactctgtctagaccccatgactaatgactcttccacattggacagcctttttagcagcctccatagcagtaatgatacagtgcccattaaattcaagaagtgctgctatggatattgcattgatctgctggaaaagatagcagaagacatgaactttgacttcgacctctatattgtaggggatggaaagtatggagcatggaaaaatgggcactggactgggctagtgggtgatctcctgagagggactgcccacatggcagtcacttcctttagcatcaatactgcacggagccaggtgatagatttcaccagccctttcttctccaccagcttgggcatcttagtgaggacccgagatacagcagctcccattggagccttcatgtggccactccactggacaatgtggctggggatttttgtggctctgcacatcactgccgtcttcctcactctgtatgaatggaagagtccatttggtttgactcccaaggggcgaaatagaagtaaagtcttctccttttcttcagccttgaacatctgttatgccctcttgtttggcagaacagtggccatcaaacctccaaaatgttggactggaaggtttctaatgaacctttgggccattttctgtatgttttgcctttccacatacacggcaaacttggctgctgtcatggtaggtgagaagatctatgaagagctttctggaatacatgaccccaagttacatcatccttcccaaggattccgctttggaactgtccgagaaagcagtgctgaagattatgtgagacaaagtttcccagagatgcatgaatatatgagaaggtacaatgttccagccacccctgatggagtggagtatctgaagaatgatccagagaaactagacgccttcatcatggacaaagcccttctggattatgaagtgtcaatagatgctgactgcaaacttctcactgtggggaagccatttgccatagaaggatacggcattggcctcccacccaactctccattgaccgccaacatatccgagctaatcagtcaatacaagtcacatgggtttatggatatgctccatgacaagtggtacagggtggttccctgtggcaagagaagttttgctgtcacggagactttgcaaatgggcatcaaacacttctctgggctctttgtgctgctgtgcattggatttggtctgtccattttgaccaccattggtgagcacatagtatacaggctgctgctaccacgaatcaaaaacaaatccaagctgcaatactggctccacaccagccagagattacacagagcaataaatacatcatttatagaggaaaagcagcagcatttcaagaccaaacgtgtggaaaagaggtctaatgtgggaccccgtcagcttaccgtatggaatacttccaatctgagtcatgacaaccgacggaaatacatctttagtgatgaggaaggacaaaaccagctgggcatccggatccaccaggacatccccctccctccaaggagaagagagctccctgccttgcggaccaccaatgggaaagcagactccctaaatgtatctcggaactcagtgatgcaggaactctcagagctcgagaagcagattcaggtgatccgtcaggagctgcagctggctgtgagcaggaaaacggagctggaggagtatcaaaggacaagtcggacttgtgagtcctaggtgaccacactgcttccctttctcagttcctgaccttcctctgagcccttgagacactttgtaatgctcttttgtaactatcgacaaaggtgtggggaagctgaggtctaggtcttcttaaaggtcaagtctgctctccctcgcctaaagtgcagcagcagctcctctcaagctcactctctaggtctccagggtaggagtgtttttctagcaagaatcttagtcaggagtaagctctgtgcgagagatctgtgaataaccagataaccccagctgccgttaaccttttcaccaggtgccacagtaatatttctggtttttagccctttctctgcactaccaacaagagataaaattgttactcacacttatgtcttactgggttgctggttttcatcgtaacacagaacgaggttatctagggttgtagcttttgatacaactccccgatctagatttattcctacattctgaatggggagcaggtaagagcagagcacctcccactgggggtggggtatttaaaaattaactcattagtatcataaacgtcaaggattgattggaccaggcaagagccatgtttttgagaaggttctggatctctgactccatcctgactgtttagtaagagcatgcttacaccctactgtgaaaaggggaggggatgtggtaagcggaaacagaagacaggcagcagaggcattaaaaatgcataccatgctttcagaacaaaagctctgggccagaaaggcaatttggctaaaaaatgaataagactacttctaatgtaactaagcatctccactatggtgtgtgccttttataaaggaaaagagagaaaaaggcaaagcaaggttgtggccttaggttggacctggaatatcccttattgcctataatggaatatgtgacactgtgggtgaaatgttctacacaccacacactaggccattttcagatcagcagtcacccatcgcttagcatagaaatcccaaaacctccagcccgggaacactataagcttcgaccattcaggaatctgccctgcactttgcatatctgtatagaaaatcaagtcaatcccccatcctcacacccactcatctctgaggagctatgaactggttttggtccctctaatgatcctccagcctcatctaatgccccccaaagactgatacaagtaacctcccctctgcttaggtgtcactttctcagcatatcaagtttaggcagcaagggaaaggaatatgggtcagttctcaaatgtcaatgtagataagagtcatctagtagagaactcatcagagtgcggattgccaagacccttctccagagattatggggttgggggtggaggtctagaggtgagctcagaaacctactgttaaccaacacccccaagtgactgacacaggtggtctaaaaattacttttctagaaacaccattctggaagtttggctgcccacaggcaggaggagaagcatgaagagaaaacctgtttgagaagttttgttttgttttgttttgctttttaataattttagcacacatctgctgactctccttcaacatcctcacccccacccctgggcaccatttaggacaagacttccttatttatcaattacttgatttatcttctcaggactcattgttccacccccaaccaatttgaatgcctacaataagttcaggagctgtgccaagcactttcctcttttacagctggagatcactggaaaggtgtctcagtcacaaaacttctccctctactactggatgaaatgtctgcatttccaccaaaatctacccagtcacccagggaataacaacttaagctgtagttagataacacctagtgattaattggctgagaaaaccctggagtggagggaggctcagagatactgatatggatgtgggagggctctaaagttagaggtcaccaactccacagatgaaacagttcaataatgaggaaacaggtgagccctgaaaacacaaaaggacagttctgtgttgaaacaccccatcccctcacgttctcaccccaggcccagaagtaggttgcaactgcctttggaagattttgccccttagccatccccacccacttgtaccagctaagaatgctggagactctgccaccatgctctgcgtgcccctgaacctctgtgcagcccggaaggctgatgtacaggtgtacctcaatccacattacagccatgctcctaatgtacatggacatttttgtaactcagctcatattctgactgtatttgagaagctggctgtttaagggaacccagaagtgaattcttttgtaaagtaaagcacccttttgtaatgcaattaattatcccttaatgtatctgttttgtaagtctgcatttttgtatatcggatttaccttaagcttctctagtgaggcattctgagcagtggtgatcacatgccagatcgccctgcctatccacaaagtagatgaccaatgcacgctcctcaaacatctttggaggaactacctggccaaaacactggccaggatgcagcaagcagcagcaggggctgacagcaggcttactgccatcaacattgcttgaaatgcctctatgttctgaataaagaaaaaccataattgcttgtggtgaaacgaagcagtcttcatgttaagtagcaatggttatttttattggtagtaactgaacagtgttttgcaatttgtgaaacagtgtattgtgttttgtaaaatgatgtcatgaaatggtgggtccttggaaacctcctttccgttcagctctgcctctgttctttcaactcctttgaggctcaaaaaaaacacaaagatcagaagccttcagatagagggtggtattctggtaaagaagaaagagataagggacgctaccttgcttttctggcacaggaagcacatgataaagcatgctcagatgagctggaacagatatagctacctggttcgtgtaaataagaataatcaaggccccagagtgtgtatgcttccaggtggaggagaaaggggaatctcccaaaatttaaaaacaaattggaagaataaccaggacagccaagtgaagcagccacagggacccaagcagtcgaggtctttaatgtgcctggagatgactctctgctattcatgaatcttgctattgcacaaaccctatcaagagctgctgcttcccttccagccagaaaagtggtaagcggagcaagtgccaagcagaacagaccttatcatctgggtaacagacttctcagtgttggtgctgtgtctgttagagccttagagcaagttaagcacttccttggtgtgggtaaagaataaaggggaaagaaactactttagagcctctttttctcccaactcatatttttgataggaaaaacagaaaacccatccagttcttcagaaattgctttctaggcattaatactactttactatctatactgtttagttattcctttctttacccacctaaactatccatctaatccaggattccctcactctttttttttagttactaatcattttatgaaaataatgtatttataagtattttcttaaggtttgtgaagagtatttgcattgtgtcttcattttaatgtgtttgcaatcgctccgctccaggaagaacggaaatgctgtcttgtgagcatgaagtgaacgggctgttttgctccagccacttttcttgtacaaccacatggatggattagatgtcctcaggtcttttccatcttcagtttctatgactgtggaataaatgttcagatagaaacttca

SEQ ID NO: 32 - DNA, 666 bp, Homo sapiens
  source 1..666: mol_type = genomic DNA
ttgaagagatgagtgcggggctcatctatccctggaattgtctttcccacaatccctgacacagaatatgagccatacaggaattctgaagaaatgggtctcttgccacctcccagtaaaagattattttttaaaaaaaaaaggctctgctttgacctgaagtattttatctatcctcagtctcaggacactgttgatggaattaaggccaagcacatctgcaaaaaagacattgctggaggaggtgcaaagagctggaaaccaagtctccagtcctgggaaaagcagtggtatggaaaagcaatggaaagagcattttgaaaatgccattccactgttttctggcctttatgatttctgctgagaaatccactgttagtctgatggggtctccttcatagcaccaatgacctgaagagccttgttgaaggaagactccatctgatgactcagagcaagtattttttagtgtgttattgttattagcagaaagagggccataaaatacatggggcaagctgaatatatcttaggcaaaagaagaaaatattcaaattcttatgttattttatctaattattttatctctttttgtgtgtgacttataatgtgtgtattgtattaataaaagtatataaacatgtagtttacaaaaaaaaaaaaaaa

SEQ ID NO: 33 - DNA, 6242 bp, Homo sapiens
  source 1..6242: mol_type = genomic DNA
gagcacaaagcggggcgcaccgcgggcgccggcaacgagccggtgaacgaggcgaggcccgtgcgcccgcggctgcaagcgcccgcctggcggggagaggggccgacggcgtcagcccgggcggcggcatccctaggcgcctggggcgccttcctccggacctggccgctcgctgccccgccctctgcaccccacttctccgaccctccttcccagtcctgcctccccctgccctggcctctgagagccgactgagcccagccccgtgcagcagcggttgcctgtgtcgccgcctagtctccggtcttggtgctctcccgggggtgccccaaggagccagtgcgcgctgcgggctgggaaggaggcgccgctcagctagtcctcctcctcctcctcgtctttctcctcctcctgctgctgctgccgccgccgccgccgtgggtgccgggtccgcgcgcaccccaacacccccaccagctgggcctcgggggagtccctgcctggcaagctggggctcggggaaaagtccagcagcgagagggccgcgtgtcccgggacggttccaggggagcccgcgcggaaacgcggaccaggcgcttcggcccggatgccggatccaggagaaggggacctcgcttgccgtgtccaccgccgcggccccagcgccaagaacagagcctggccgtgagtgaacgactggccggggtacaaatggttctgaagcacttgaagctcacaggcaaaggctaagagtctaaatcccatcccgccgcacatctccagacttcaatttggctgaaataattcatgccacggacctgtgcacatgcctggaattgagagacacagttaaaagactccaagttgctttctgccttttgaaaactcctgaaaaccatccctttggactctggaattctacacagctcaaccaagactttgcttgaatgtttacattttctgctcgctgtcctacatatcacaatatagtgttcacgttttgttaaaactttggggtgtcaggagttgagcttgctcagcaagccagcatggctaggatgagctttgttatagcagcttgccaattggtgctgggcctactaatgacttcattaaccgagtcttccatacagaatagtgagtgtccacaactttgcgtatgtgaaattcgtccctggtttaccccacagtcaacttacagagaagccaccactgttgattgcaatgacctccgcttaacaaggattcccagtaacctctctagtgacacacaagtgcttctcttacagagcaataacatcgcaaagactgtggatgagctgcagcagcttttcaacttgactgaactagatttctcccaaaacaactttactaacattaaggaggtcgggctggcaaacctaacccagctcacaacgctgcatttggaggaaaatcagattaccgagatgactgattactgtctacaagacctcagcaaccttcaagaactctacatcaaccacaaccaaattagcactatttctgctcatgcttttgcaggcttaaaaaatctattaaggctccacctgaactccaacaaattgaaagttattgatagtcgctggtttgattctacacccaacctggaaattctcatgatcggagaaaaccctgtgattggaattctggatatgaacttcaaacccctcgcaaatttgagaagcttagttttggcaggaatgtatctcactgatattcctggaaatgctttggtgggtctggatagccttgagagcctgtctttttatgataacaaactggttaaagtccctcaacttgccctgcaaaaagttccaaatttgaaattcttagacctcaacaaaaaccccattcacaaaatccaagaaggggacttcaaaaatatgcttcggttaaaagaactgggaatcaacaatatgggcgagctcgtttctgtcgaccgctatgccctggataacttgcctgaactcacaaagctggaagccaccaataaccctaaactctcttacatccaccgcttggctttccgaagtgtccctgctctggaaagcttgatgctgaacaacaatgccttgaatgccatttaccaaaagacagtcgaatccctccccaatctgcgtgagatcagtatccatagcaatcccctcaggtgtgactgtgtgatccactggattaactccaacaaaaccaacatccgcttcatggagcccctgtccatgttctgtgccatgccgcccgaatataaagggcaccaggtgaaggaagttttaatccaggattcgagtgaacagtgcctcccaatgatatctcacgacagcttcccaaatcgtttaaacgtggatatcggcacgacggttttcctagactgtcgagccatggctgagccagaacctgaaatttactgggtcactcccattggaaataagataactgtggaaaccctttcagataaatacaagctaagtagcgaaggtaccttggaaatatctaacatacaaattgaagactcaggaagatacacatgtgttgcccagaatgtccaaggggcagacactcgggtggcaacaattaaggttaatgggacccttctggatggtacccaggtgctaaaaatatacgtcaagcagacagaatcccattccatcttagtgtcctggaaagttaattccaatgtcatgacgtcaaacttaaaatggtcgtctgccaccatgaagattgataaccctcacataacatatactgccagggtcccagtcgatgtccatgaatacaacctaacgcatctgcagccttccacagattatgaagtgtgtctcacagtgtccaatattcatcagcagactcaaaagtcatgcgtaaatgtcacaaccaaaaatgccgccttcgcagtggacatctctgatcaagaaaccagtacagcccttgctgcagtaatggggtctatgtttgccgtcattagccttgcgtccattgctgtgtactttgccaaaagatttaagagaaaaaactaccaccactcattaaaaaagtatatgcaaaaaacctcttcaatcccactaaatgagctgtacccaccactcattaacctctgggaaggtgacagcgagaaagacaaagatggttctgcagacaccaagccaacccaggtcgacacatccagaagctattacatgtggtaactcagaggatattttgcttctggtagtaaggagcacaaagacgtttttgctttattctgcaaaagtgaacaagttgaagacttttgtatttttgactttgctagtttgtggcagagtggagaggacgggtggatatttcaaatttttttagtatagcgtatcgcaagggtttgacacggctgccagcgactctaggcttccagtctgtgtttggtttttattcttatcattattatgattgttattatattattattttattttagttgttgtgctaaactcaataatgctgttctaactacagtgctcaataaaatgattaatgacaggatggggttcccctgtgcttttaccagtagcatgaccccttctgaagccatccgtagaaagtactttgtcctccaaaaagctaacatacggttttgaagcagcattgaaacttttgtagcaatctggtctatagacttttaactcaagaagctaaggctagacttgttaccttcgttgaatgatgttagttgactgtactgtaatgttgtatcaactgaattgaatgtttgcctttaaacaatgaattttctttttctttccttttttttttttttgttgtaatagttaaagaggcttagaacaagctaacaggcaatagaaatatgtatatcagattttttaatgtaacaaactacatgttaattgttatcttattctttttatctttagtagacacttttaaaagaaaagacaagtttgttgtgtttaactcaccaacacgtggtgtataatgaagacagaactataataaattagttttgttctgattttttagaacacttgcaataatgtatcatttatagttcttgctagttgcagtggtaatatttttcacatccataaaaacaactaccaaaataaatcagctgtagcatgttgctttttaaagctaggccctaaaaggttttaattctttttctaagggaagaaatgtctattttaattaagatattttaatgaacaggatttctgtattttaaatagtactgactagcacctaatgggcagtgggagggtggttcatatgaagaaaaaaaggtgtattgttgtatcccatgcataaataaaggtaaatatatatatacaccaatatattcatatatactcacacacatcccaacctgtcacacacaatgcgtgtgtatatatatatgaatatattggatatgtcatttctgtaagagttttgttaaaacctgattttcttttgtagtatccacattcttcatcaaagtacaaaaacgtctgtggagtgtcacaaactgtatgacatgttatttctttttaacagttgtctatatgcttagacccgtgttagtctctatatctgtgtggcaatatctgctgagacaagtaaatgattaatagaaaacaaaacaacttccgtacagttcaaacttttcatccaaatatatatagacagttttggagaattgtttcaagattatagaggaaacatgtaacatttagagcagatggaactaggtttaggtagaaggccagttccacaaagggcagagggagggatgggatttaataggtaaagaagaacccatttgaaaataaaggttgtttcaaaaggcagctgccgccaggcacacagcattccatcagacaggtgccagacaagcaaaagcaaggaagttggcagaaagaaagtccaggtgatgtaggttgaggtatttctttctttggcagtatcttgcttttgtgaatcactttattaaaaaaaatcactttcttcccattcttaaggggttttggcaaacagaatttcagatcttgaaacaaatgagctgcaacagaaaaataagtacctgagctcgaggtatcccttcccaaagccttcacttattggtgagaaaaacctgggcccagggagggcttgcagcttacccagttcggactcctgccagttcagcgctctgcactccattgattgttctaggcccggcagcctgttaggttcctgtgcagggcccctcttttgcagttctgaataacctctagtgcccctgcttcaaaaatgcagtacatcctcaagttcccatttactcaggatacattttagcacagggcagatcagacggtgtctctactgttagtattgcaaagtatgtatgggaaacacagagaattggagctgcgttgaatgcaaacttgaggtgtttcccttgaggaattcttgtcttcaaacgtctgcagagtaatggaccatgttacaactttcctgttcatctgtgaaccatgaaaatggatggcactgatgcattagaccctcagcagcctgcaattgcaaatctgcgaggtttcattcggcccataaagcaaacatttgaacttacacagaatgagcacttaaatacgggtgcaataaatgaagggaaaaacctcagccgtttctccattctgaagatatagcaagcaccgggaaatctaagatttttcatcaacaatatcttctgccagcccagtttggggggaaaaaccccttttacatttttcttcagtaaagtactggaacttacttttccctgtctgtgctaatgagctgattttcagctgatagaaaacaaaatgatagagtacttttttccttggccaagtattttctcatttgtatttaatttcataaattagacagccagtgaaattagacctcaaactaggtcctgatggataatgaatgttatgtcacctttaacagtgaagtggttattataggtcactttctaatttcatattttcccttttgctttctgctgccttcagggtatatagtgtatctctaacctgatttttcaaggttatttttggagcagtttcttaaaacaggcattccctaacttgctcatttaattaatgaaaaattgaactgatgccatggatataaaaacaaatgtaatgtttgattgtcagtgtttctgatttggcaaaaaggaatcatctctatttttttgcaaacaatatcaaagtgcatattttctctca

SEQ ID NO: 34 - DNA, 2381 bp, Homo sapiens
  source 1..2381: mol_type = genomic DNA
ggaaacgcggagcgcgcgctcccagcgaaagcagcagggcagggatctgcgttggaggaagggactgctctggtgctagaatgctgtgcgtcggaaggctgggcggcttgggagccagagcagcagctctgccgccccgccgggcgggccggggaagcctcgaagccgggatccgggcccgaagggtcagcaccagctggtctcccgtgggcgccgccttcaatgtcaagccccagggcagccgcttggacctgttcggcgagcgccggggtctttttggagttcctgagctgagtgccccagaaggatttcatattgcacaagaaaaagccttgagaaagacagaattgcttgtggaccgtgcatgttccaccccacctgggccccagaccgtgctgatcttcgatgagctctcggattccttatgcagagtggccgacttggctgattttgtgaaaatcgctcaccctgagccagcattcagagaagctgcggaagaagcttgtagaagtattggcaccatggtagagaagttgaacacaaatgtggatttatatcaaagtttgcaaaaattactagctgataaaaaacttgtggattcccttgatccagaaacaaggcgagtggctgaactgtttatgtttgattttgaaattagtggaatccatctagacaaagaaaagcgtaaaagagcagtggacctcaatgttaaaatcttggatttgagtagtacatttcttatgggaaccaattttcccaacaagattgagaagcatctcttaccagaacacattcgtcgtaactttacatctgctggggatcatatcataattgatggtctccacgcagaatcaccagatgacttggtgcgagaagctgcttataaaatttttctttatcccaatgctggtcaattgaaatgtttagaagaattgctcagcagcagagatcttctggcaaagttggtggggtattccacgttttctcacagggctctccaaggaacgatagctaaaaatccagagactgtcatgcagttccttgaaaaactatctgacaaactttctgaaagaactctgaaagattttgagatgatacgagggatgaaaatgaaactgaatcctcaaaattccgaagtaatgccctgggaccccccttactacagtggtgtgattcgtgcagaaaggtataatattgagcccagcctatattgcccgtttttctctcttggagcatgcatggaaggcctgaatattttgcttaacagactgttggggatttcattatatgcagagcagcctgcaaaaggagaggtgtggagcgaagatgtccgaaaactggctgttgttcatgaatctgaaggattgttggggtacatttactgtgatttttttcagcgagcagacaaaccacatcaggattgccatttcactatccgtggaggcagactaaaggaagatggagactatcaactcccagttgtagttcttatgctgaatcttccccgttcctcaaggagttctccaactttgctaactcctagcatgatggaaaatcttttccatgaaatgggacatgccatgcattcaatgctaggacgtactcgttaccaacacgtcactgggaccaggtgccctactgattttgctgaggttccttctattctgatggagtactttgcaaatgattatcgagtagttaaccaatttgccagacattatcagactggacagccactgccaaaaaatatggtgtctcgtctttgtgaatctaaaaaggtttgtgctgcagctgatatgcaacttcaggtcttttatgccactctggatcaaatctaccatgggaagcatcccctgaggaattcaaccacagacattctcaaggaaacacaagagaaattctatggcctaccatatgttccaaatactgcctggcagctgcgattcagccacctcgtggggtatggtgctagatattactcttacctcatgtccagagcggtcgcctccatggtttggaaggagtgttttctacaggatcctttcaacagggctgccggggagcgctatcgcagggagatgctggcccacggtggaggcagggagcccatgctcatggttgaaggtatgcttcagaagtgtccttctgttgatgacttcgtaagtgccctcgtttccgacttggatctggacttcgaaactttcctcatggattctgaataaaagaaacactctacacctcttaaatcaaggtcatgtagataatgactttgttataaatgctacagctgtgagagcttgtttctgatttcattgttcgcttctgtaattctgaaaaactttaaactggtagaacttggaataaataatttgttttaatta

SEQ ID NO: 35 - DNA, 1311 bp, Homo sapiens
  source 1..1311: mol_type = genomic DNA
agcatgtaacctggcctgcatccaggaaatagaggacttcggatccttctaaccctaccacccaactggccccagtacattcattctctcaggaaaaaaaacaaggtccccacagcaaagaaaaggaataggatcaagagatacgtggctgctggcagagcaagcatgaattcgatgacttcagcagttccggtggccaattctgtgttggtggtggcaccccacaatggttatcctgtgaccccaggaattatgtctcacgtgcccctgtatccaaacagccagccgcaagtccacctagttcctgggaacccacctagtttggtgtcgaatgtgaatgggcagcctgtgcagaaagctctgaaagaaggcaaaaccttgggggccatccagatcatcattggcctggctcacatcggcctcggctccatcatggcgacggttctcgtaggggaatacctgtctatttcattctacggaggctttcccttctggggaggcttgtggtttatcatttcaggatctctctccgtggcagcagaaaatcagccatattcttattgcctgctgtctggcagtttgggcttgaacatcgtcagtgcaatctgctctgcagttggagtcatactcttcatcacagatctaagtattccccacccatatgcctaccccgactattatccttacgcctggggtgtgaaccctggaatggcgatttctggcgtgctgctggtcttctgcctcctggagtttggcatcgcatgcgcatcttcccactttggctgccagttggtctgctgtcaatcaagcaatgtgagtgtcatctatccaaacatctatgcagcaaacccagtgatcaccccagaaccggtgacctcaccaccaagttattccagtgagatccaagcaaataagtaaggctacagattctggaagcatctttcactgggaccaaaagaagtcctcctccctttctgggcttccataacccaggtcgttcctgttctgacagctgaggaaacgtctctcccactgtttgtactctcaccttcattcttcaattcagtctaggaaaccatgctgtttctctatcaagaagaagacagagattttaaacagatgttaaccaagagggactccctagggcacatgcatcagcacatatgtgggcatccagcctctggggccttggcacacacacattcgtgtgctctgctgcatgtgagcttgtgggttagaggaacaaatatctagacattcaatcttcactctttcaattgtgcattcatttaataaatagatactgagcat

SEQ ID NO: 36 - DNA, 8615 bp, Homo sapiens
  source 1..8615: mol_type = genomic DNA
ggagaccgactcgggatctgtccgagcaggaagccagcctcagcccggccgctgtcgccgccctgtcctggtgcccgtccgcgtcgtcgccctcttcactggccctcatcacttctcaccgcgccctccagcttcacccgtacaggtagccccgccgccgcgcacctgccttcgctcccgcaccggtgacagtggatagtggaaacaggagatcgtggatcctccttcaaaaatggaggatggaaagcccgtttgggcgccacaccctacagatggatttcagatgggcaatattgtggatattggccccgacagcttaacaattgaacccttgaatcagaaaggcaagacatttttggctctcataaaccaagtgtttcctgcagaagaggacagtaaaaaagatgtggaagataactgttcactaatgtatttaaatgaagccacactgctccataatatcaaagttcgatatagtaaagacagaatttatacatatgtcgccaacattctgattgcagtgaatccatactttgacatacctaaaatatattcttcagaagcaataaagtcatatcaaggaaaatctcttgggacaagaccacctcatgtctttgcaattgctgataaagcttttcgagacatgaaggtgctcaagatgagtcagtctatcattgtatctggagaatcaggagccggcaaaacagaaaatacaaaatttgttctaagatacctgactgaatcctatggaacaggtcaagatattgatgacagaattgttgaagctaacccactcctagaagcctttggaaatgcgaagactgttcgcaacaataatagcagtcgatttgggaaatttgtagaaatacattttaatgaaaagagctcagttgttggaggatttgtttcacattatctcctagagaaatctaggatctgtgttcaaggcaaagaggaaagaaattatcatatcttttataggttgtgtgctggtgcttctgaagatattagagaaaaacttcatttgagttcaccagataattttcggtatttaaaccgaggctgcactagatactttgctaacaaagaaactgacaaacagattttacagaaccgcaaaagtcctgagtaccttaaggcaggttctatgaaagatcctctgctagatgaccatggtgattttattagaatgtgcacggctatgaaaaaaattggtttggatgatgaagaaaagcttgatctcttccgggtagtagctggcgtcctgcaccttggaaatattgattttgaggaagctggcagcacttcaggtggttgtaatctgaagaataaatctgctcagtctttggaatattgtgctgaattactgggtttggaccaagatgatcttcgagtaagtttgaccacaagagtcatgctaacaacagcagggggcaccaaaggaacagttataaaggtacctctgaaagtggagcaagcaaacaatgctcgtgatgccctggcaaagacagtgtatagccatctttttgatcatgtggtaaacagagtaaatcagtgttttccttttgaaacatcatcctattttattggagtcctagatattgctggttttgagtactttgagcataacagttttgaacaattttgcatcaactattgcaatgaaaaacttcaacaattttttaatgaaaggattctgaaggaggaacaagaactctatcaaaaagaaggtttaggtgttaatgaagtgcattatgtggataatcaggactgtatagatttaattgaagccaaattagtgggaatactggatattttggatgaagaaaatcgccttccccagccaagtgatcaacactttacatctgcagttcaccaaaagcacaaggatcattttcgactcactattcccagaaaatctaagctggcagttcataggaatatcagagacgacgaaggcttcattatcaggcattttgcgggggcagtgtgctatgaaacaacccagtttgtggagaaaaataatgatgctttacatatgtctcttgaatccttaatatgtgaatccagagataagtttatacgggaattatttgaatcatccacaaataacaacaaagatactaaacaaaaagcaggaaaacttagcttcatcagcgtgggaaacaagtttaagacacagttaaatttgcttctggataaacttcgaagtactggagcaagctttattcgttgcatcaaacctaacttaaagatgacaagccaccactttgaaggtgctcaaattctgtctcagcttcagtgttcagggatggtgtctgttttggacttgatgcagggtggttacccatcacgagcttcatttcatgaactctacaacatgtacaaaaagtatatgccagataaacttgcaagattggatccaagactattttgtaaggctttgtttaaagctttgggcttaaatgaaaatgactacaagtttgggttaaccaaagtattttttagacctggcaagtttgcagaatttgatcagatcatgaagtctgaccctgaccacttagcagagttggttaaaagagtcaatcactggctcacatgcagtcgctggaagaaagttcagtggtgctcactctcagtcatcaaattgaaaaacaaaataaaatatcgagctgaagcctgcattaaaatgcaaaaaactattcgaatgtggctttgcaagaggagacacaaacctcgcattgatggtctggttaaggtgggcacactgaaaaaacgacttgataaatttaatgaggtagtcagtgtgttgaaagatggaaaacccgagatgaataaacagatcaagaatctggaaatttctattgatactttgatggccaaaattaagtccactatgatgacgcaggaacaaatccagaaagaatatgatgcactggttaaaagctcagaggaactcctcagtgcattacagaaaaaaaaacagcaggaagaggaagcagaaaggctgaggcgtattcaagaagaaatggaaaaggaaagaaaaagacgtgaagaagacgaaaaacgtcgaagaaaggaagaggaggaaaggcggatgaaacttgagatggaagcaaagagaaaacaagaagaagaagagagaaagaaaagggaagatgatgaaaaacgcattcaagctgaagtggaggcacagctggcccgacagaaggaggaggaatcccaacagcaagcagttctggagcaggagcgcagggaccgggagctggccctgaggattgcccagagtgaagccgagctcatcagtgatgaggcccaggccgacctggcgctgcggagaaatgatggaacaagacccaaaatgacaccggaacaaatggccaaagaaatgtcagaatttttgagtagaggtcctgctgtactagccaccaaagcagctgctggtactaagaaatatgatcttagtaaatggaaatatgcagaactacgtgataccatcaatacttcttgtgatattgagctcctggcagcttgcagagaagaatttcataggagactaaaagtgtatcatgcttggaaatctaagaacaagaagagaaatactgaaacagagcaacgtgctccaaagtctgttactgattatgattttgcaccatttttgaacaattcacctcagcaaaacccagcagctcagattcctgccaggcagcgggagattgaaatgaaccgacagcaacgcttcttccgcatcccattcatccgccctgccgaccagtacaaagaccctcagagtaagaaaaaaggctggtggtatgcccattttgatggaccatggattgcccggcaaatggaactccatcctgacaagccacccatcctacttgtggctggtaaggacgacatggagatgtgtgagctgaatcttgaggagactggcctgactcggaagcgtggtgctgagatcttgccaagacagtttgaagaaatctgggaacgctgtggaggcatccagtaccttcagaatgcgattgagagcagacaggctcggcccacctatgcaacagccatgctgcagagtctgttaaagtagatgttgcacaccagccttacagctgggagcctttgccatggtacttaggtagggtgtgtgcccccagatttaaccattccataatcatgttagagttacttctataaagtgaacagattttattaatcacggcttttggtgaatttgtttaaggttaattatggtagcaaattttggacctaaacattatttttctgtatcccgctgtaattcccaaaactctcattattctctaactattacacatgggcatattctgatgtttctcatcctttgccagaagactaccttacatccatcgtaattgttctctaggaaaagagaacttttttcaaaattcaaaatactttttaaggatggcacagtaccatataactggagtaataaaacatgagcttacattcttacaataactaaaccacttaaaatgatcaaggcactaatgttttggtctgaaaagctgtgtactttatagacattttcagacatttttggaaatttccattaaaggtggaaaatctatttttttcctcctttgcagtgtcttagtttgaatgaaacacttcgaagttctagaattctagaaagagccttaatgtatttgatgtattctgtgataagaggtactaatagtatccagcacagatttgcttttctttgctagcacaatgtgtgttgctgtcagaatattctttttatattctgtggaaaaataaaggaaattcagattgtttaaatgcctaaaagttttgagataagttttgtttcaattagaaaaggaaataggttttaggtggcatagtggcttaactggactgaattcaaatattctttcaacttcatctcaatagtgatttttgtatcagaatcttgtccaagttgtttcattgatttagtaagtgttctgcttccaacatctttctttttaagaaattcctagtgtcttttttggcctttgaggttttggtaattgtagacctgtttcataagctttgtaattcagaaatccttgtatttagtaagtgcttgttttacataactgataattttaaaatgttttctttgtgtgctgttagtattgattcaaatgtcagcagctttaagcctaatatttatgactttcacatttggaatttaaagacaaaaatacatcaaggagttatgctgacataattctaaggagttttgttgtattttagaataaaattataaagtaaaatgattctctgtactgctttttcccccagtttttagagaccctaacctttgaaatgaaattccagtgatttcttttttccctagaaagattacctcagttagggaagtatttcccagctgactagtgtttgtgagccacagacactgtcttcagaattgcttctctcatgtcttagtagagaaatatttatttattatgatacattcaaatgattgtcaagttaaattaaatggttgtgtctgtgctattgagaatgcaaatgtgattatcttttgaaggctgtattactgcatagcttcacccaccctcgggtcatttcgtccctgtgattggggacagaaggtgtagctactgaagtaaatgacctattctctctcttccatctctcgcctttaactggtgtttttatttgtgtaggatagtgaatgataagcttttttcctaaccagtagtgagtaaagttcttgaacaaaatttagtagccaaattgttttttaatgacatgtctctttagtacaatagttttgtgtatcttttagatacattaataggcactagatggaaaattaaagagttaaacatatttaaatgagagaatctaatgtttcagaaatttgtaagaaatgtatcacagcaaagggttgttataagtccttagtttttgactctaatagttaatacaattatagttaatcttaagccataatgtttctaatcatgtcacacagctgtcctagaacttatctatttaaaatagtttcctgagttaattttggccagcagggcaactgccctaattcagatagatttacagtaacctacgtacagtagatgcacatacacacagacacccctttgctggagaaacttaggaccctgtcagccttttaaaggaaacagcaggagtggtgtcctaaatgatgttcatgcagctgctttaccatgttcacagtcaagcccatgcatgccaggttaaaactgtggaaatcaaaagtaaattcactcatattttaatcattttaactgagatttaaaattagaagtttaaaccactatatataaagaactaatcttttcttaataccagttctttccatagcatatgctttgcaaaggcagcatgcataaaatatttaaaatgagaggacagaatgttttcacatttgattcaattttaatataattcctaattgtggtaacacagttgagatatgtattatgagttatgggaactaattgagaaaaggaagttactctaatccacgtatgttaagagaatattgagttttcttagttgtaaagttggggagatggcaccttctcagaggattgtgaaaatatgaggaagaaacaaaacagtgcatgtaggagcacagggccacacaaaggcattctattgttatgctcattctgcttctgtaatgacttttcataggtcattcttgtgaaccattttgttttgcaagcaaccaaggaaagaacatcttaagtggaaaatcagtggtggttgtgaacacttagagaatagcaatccacaggcaagaataatggtattgtttgtagagctttattaattggatattttttaaaagacattttcattcacaggtcattactatggttctcagcgatccaaatatgtagatcattggtttttttttttacctgaagtagcttaagagtacttggatcagtagaataaatatttattgaatcaatcagtcagccaattaatatgatgttagtgatagacctgcctccttttatggaagaggtaacagatccagagaggtcaagtaatttagttgtagactgaaaaatatatcaaagcctttgctgcaatcatatgtaacaaaaagaaccaaaacaaacactttttagtggcacctgtggatttacaaagggttgcctctctgtcattccacaacttcagaaggtgtgacaggttttccctatttatcattaccaataataacaagtattgagagttttaaaatttctcccagaagataaactaacaaggatggaaggggagggcaaaggatatctaaacatgagaataaggacatgttagagggggggaaacagttgtaacaataaggaaagagaagagcaacagtggaagagacaggttgtgtgcccctaaagattctgcacccccagtttggaaacactgatacattttaggacacagagcactcctagatctctacgaaattttagaatgaataatgtgtaatttataggatcagaacgtatggttattaaaacttggatcaagatatgcccggtgtatacattcttagcacataggaatggcactgccatactggagaaggtcagcagtaaataggcattctgtacataagcctcatggaagggtaagatggagagactggcagaagtagcacctactctgctgggagcacttctctgagtacgctttagttcaattcaaatcactgtattctttccccattgctaacctaatatatgaaacaagcttagctgtctcagaagtttttcaagagatgatcaggaaaaattaatgcacattcaaaaggagaatcttcagtacaaatttgtttttttaaaaatagatttagggctgggcgcggtggctcacgcctgtaatcccagcactttgggaggccgaggcgggtggatcacgaggtcaagacttcgagaccagcctggccaacatggtgaaaccctgtctctactaaaaatacaaaacattagccaggcgtggtagtgggtgcctgtaatccctgctacttgggaggctgaggcaggagaatcacttgaacgcgggaggcagaggttgcagggagccgagatggcgccattgcactccatcctgggcaacaagagcgaaattccatctcaaaaaataaaatagatttagggggtacaagtgcagttttgttacatgggtatattgcatagtggtaaaatgtgggcttttagtgtacctaacacccagagaagcatacattgtgcccagtaggtaatttttcatccctaaaccttttctcagcctcccactttctggagtctccaatgtcggttattccactctttatgtccatatctacacattcaatcctaatttgtaccaagtagcatctcacctttaaatcacaggcttattagttgggtgttttctttttacttatgaaaattcatctagtcaaactgtcaattaatttttcctcatttcattaaaagtgtatatctaatgctttctctaaaaattgatgtactggaaatacaaataaataaatgctccctgtgtagaatttc

SEQ ID NO: 37 - DNA, 3214 bp, Homo sapiens
  source 1..3214: mol_type = genomic DNA
tctatggccagtctggccgccatgttggagctttcgctgttcggggcccggcctgctccgggccctcccagcgtcgccgctagctgacttagggctcccgcccacactgcgcacggccttgctgagggccggctcgtagtgggaggcttcgcgttcacctgaacatttccgcctcctctggatgggggatgagaggcggggctgatgcatcgcagcagtcctggaccctgacttcggacttggagaagaaacgccgaggaggaacgggagttagggggttagcagcttggcgcgctgggagccggccacccctggtctccggacttcacttcccaggaggcctcgcgcgcgactggaagtgctgcgagcctataagaaggcgaggcggcacccgccgctctgctctggggcggcattgccagccggctgtaggcattcagggcagtgtcttctgcatctcctaggaacctcgggagcggcagctccggcgcctggtagcgagaggcgggttccggagatcccggcctcacttcgtcccactgtggttaggggtgagtcctgcaaatgttaagtgatttgctcaaggtgcccatttcgcaggaattggagcccaggccagttctctgagcctatcattagggctaaagaggaagagaaagcagaagtctcagggctggagacagctgaggggcatcagctggttcaaggtgtggagtgtgggcagtggagtgaaggcctggttacctctccagtgaagattagccccatagagcttactctatggggaagaacttttctgactccttcctgtctagggagtgcgtgatcagaatggtgtctggacggttctacttgtcctgcctgctgctggggtccctgggctctatgtgcatcctcttcactatctactggatgcagtactggcgtggtggctttgcctggaatggcagcatctacatgttcaactggcacccagtgcttatggttgctggcatggtggtattctatggaggtgcgtcactggtgtaccgcctgccccagtcgtgggtggggcccaaactgccctggaaactcctccatgcagcgctgcacctgatggccttcgtcctcactgttgtggggctggttgctgtctttacgtttcacaaccatggaaggactgccaacctctactcccttcacagctggctgggcatcaccactgtcttcctcttcgcctgccagtggttcctgggctttgctgtcttcctcctgccctgggcgtccatgtggctgcgcagcctcctaaaacctatccacgtcttttttggagccgccatcctctctctgtccatcgcatccgtcatttcgggcattaatgagaagcttttcttcagtttgaaaaacaccaccaggccataccacagcctgcccagtgaggcggtctttgccaacagcaccgggatgctggtggtggcctttgggctgctggtgctctacatccttctggcttcatcttggaagcgcccagagccggggatcctgaccgacagacagcccctgctgcatgatggggagtgaagcagcaggaaggggctcccaagagctcctggtggtgcagcctgtgctcccctcagaagctctgctcttcccagggctcccggctggtttcagcaggcgactttcttccaatgctgggcccagacttcttgcctgggtgctggcctgccctctccggccgcttgctgcctgtctgctttccttggtggctttgcctgggtgctgggcctgccctctccggccgcttgctgcctgtctgctttccttggtggctttgcctgggtgctgggcctgccttctctggctgcttgctgcctgtctgctttccttggtggctttggcttctgcactccttggcgtcagcctctcaggtcctccattcacacgaggtcctcctcgctctggccgctcttgctgctcctgtctgaagaaatcagactgatttcctcttaagactcctagggatgtggtgaagagctgggactcaagtgcagtccacggtgtgaaacatgagggaggtgaggtgtccgtccacttcccccataaaggtgtgcatttcagttaggctgccccgccacagagcaggcttcatctgctctgccatccagccccatctggatgtgaggtggggtggagacatcatggggtgattgcagaaagggggagtggcggcccacgcagcttctgctgaggagctgaccgctctgagctgttctgtttcgtattgctgctctgtgtctgcatgtattgtgaccgtgcggctccacctcttccagctgctgctacagctgaggcctggatcccggcctttccctgtgacttacgtgtctgtcaccggcaggcagccctacaaatcctggtgacctgctctcccaagaacagagcctgtccccagatgtcccagtagcgatgagtaacagaggtggctgtggacttcctctacttctccttgctggatcagggccttcctgcctcccgctgggcaggtctggccttgctctcttggcagggccccagcccctctgaccactctgcagctcaccatgcagctgatgccaaagttgtggtgtccagtgtgcagcagccctgggagccactgccaccttcagaggggttccttgctgagacccacattgcttcacctggccccaccatggctgcttgcctggcccaacctagcgttctgtgccatgctagagcttgagctgttgctcttcttcaggggaggaaatagggtggagagcgggaagggtcttgctcctaagtgttgctgctgtggcttttttgccttctccaaagacgcactgccaggtcccaagcttcagactgctgtgcttagtaagcaagtgagaagcctggggtttggagcccacctactctctggcagcatcagcatcctactcctggcaacatcaggccaacgtccaccccagcctcacattgccagatgttggcagaagggctaatattgaccgtcttgactggctggagccttcaaagccactgggatgtcctccaggcacctgggtcccatgaccagctccccatctccataggggtaggcatttcactggtttatgaagctcgagtttcattaaatatgttaagaatcaaagctgtctttgttcaggctgctataacaaaaatataatagcctgggtggcttaaacaaaaa

SEQ ID NO: 38 - DNA, 6043 bp, Homo sapiens
  source 1..6043: mol_type = genomic DNA
acttgtcagcccttgtctgaggcggaggcagccccgcgccgcgccggacccgagcatatttcattttctgtcattggactttgagccattagaaccatgagcaactacagtgtgtcactggttggcccagctccttggggtttccggctgcagggcggtaaggatttcaacatgcctctgacaatctctagtctaaaagatggcggcaaggcagcccaggcaaatgtaagaataggcgatgtggttctcagcattgatggaataaatgcacaaggaatgactcatcttgaagcccagaataagattaagggttgtacaggctctttgaatatgactctgcaaagagcatctgctgcacccaagcctgagccggttcctgttcaaaagggagaacctaaagaagtagttaaacctgtgcccattacatctcctgctgtgtccaaagtcacttccacaaacaacatggcctacaataaggcaccacggccttttggttctgtgtcttcaccaaaagtcacatccatcccatcaccatcgtctgccttcaccccagcccatgcgaccacctcatcacatgcttccccttcacccgtggctgccgtcactcctcccctgttcgctgcatctggactgcatgctaatgccaatcttagtgctgaccagtctccatctgcactgagcgctggtaaaactgcagttaatgtcccacggcagcccacagtcaccagcgtgtgttccgagacttctcaggagctagcagagggacagagaagaggatcccagggtgacagtaaacagcaaaatggcccaccaagaaaacacattgtggagcgctatacagagttttatcatgtacccactcacagtgatgccagcaagaagagactgattgaggatactgaagactggcgtccaaggactggaacaactcagtctcgctctttccgaatccttgcccagatcactgggactgaacatttgaaagaatctgaagccgataatacaaagaaggcaaataactctcaggagccttctccgcagttggcttcctcggtagcttccacacggagcatgcccgagagcctggacagcccaacctctggcagaccaggggttaccagcctcacagctgcagctgccttcaagcctgtaggatccactggcgtcatcaagtcaccaagctggcaacggccaaaccaaggagtaccttccactggaagaatctcaaacagcgctacttactcaggatcagtggcaccagccaactcagctttgggacaaacccagccaagtgaccaggacactttagtgcaaagagctgagcacattccagcagggaaacgaactccgatgtgcgcccattgtaaccaggtcatcagaggaccattcttagtggcactggggaaatcttggcacccagaagaattcaactgcgctcactgcaaaaatacaatggcctacattggatttgtagaggagaaaggagccctgtattgtgagctgtgctatgagaaattctttgcccctgaatgtggtcgatgccaaaggaagatccttggagaagtcatcagtgcgttgaaacaaacttggcatgtttcctgttttgtgtgtgtagcctgtggaaagcccattcggaacaatgtttttcacttggaggatggtgaaccctactgtgagactgattattatgccctctttggtactatatgccatggatgtgaatttcccatagaagctggtgacatgttcctggaagctctgggctacacctggcatgacacttgctttgtatgctcagtgtgttgtgaaagtttggaaggtcagacctttttctccaagaaggacaagcccctgtgtaagaaacatgctcattctgtgaatttttgaaagtcaacagttcaggagaagagaaggaatttgaagagaaaaaggaaaattaaaattactaattaatttttagattcaatatttatatggagttttgaaaaataatagtggccctgaaggaataaattccagctttaaaaaccaagtctgaggaaatatttggcttcataaagtaaagagacggtttggcatttattattactttttcctgtattttatgcccataaaataagctttataaaaaccaatttcctgatggactattaaattcatcttagaataaattagtgaagaatttaattttagaataaataatccaatctgaaataattataccttctttccttgttaggtagttatgagtaaatctgcaaaaggcaatgaaaatgccttaaattttatcaataacagaattattgtatttaaaaaaaaactaatacttatctttaaaatagtaaataggattttaaacagagaattttatcagtaataggtgtcagtttttaaaaaattgcttgtaggctgagcgcggtggctcacgcctgtaatcccagcactttgggaggccaaggtgggtggaccacatgaggtcaggagtttgagatcagcctggccaacatggtgaaaccccatctctactaaaaatacaaaaattagccggacgcagtggcacgcgcctgtaatcccagctactcaagaggctgaggcacgagaatcacttgaacccgggagggagaggttgcagtgagccaagatcgtaccactgcactccagcctgggtgacagagtgagactctgtctccaaaaaaaaactttgcttgtatattatttttgccttacagtggatcattctagtaggaaaggacaataagattttttatcaaaatgtgtcatgccagtaagagatgttatattcttttctcatttcttccccacccaaaaataagctaccatatagcttataagtctcaaatttttgccttttactaaaatgtgattgtttctattcattgtgtatgcttcatcacctatattaggcaaattccatttttttcccttgtgctaaggtaaagatttaattaaataattttggcctctcatagttttctctctctttaaagagaataaatagagggccaggtgtggtggctcacgcctgtgatcccagcactttgggaggccaagacgggcggatcatgaggtcaagagatcaagatcatcctggccaacatggtgaaaccctgtctctactaaaaatacaaaaatgagctgggcatggtggggcgtgcctgtagtcccatgtacttgggaggctgaggcaggaaaattcttgaacccaggagacggaagttgcagtgagctgagatcacaccactgcactccagcctggtgacagagcaagactccggctcttaaaaaaaaaaaaaaaaaaaaaaaaagagagagagagaataaatagaaaagaatgtggctgggaattgtgaatcagaagattataccccccaattgtttttcaatccccttttctcaaataataaattagttaaatcagtttctgagttatgccactggctgatgaagagttgagaggtctctttgcagaatgatctttttgtttcgttttgtttcttcttctgcatttaaaaattaaaagattggtttgaggatgtgatgaaattgagactttttgtggttttctctcaataataagtgaaccaatttcaaatgtgatcacaaagtttggaaagcttttattcacagaggttgggtagtgttgggaggggagtttaattactcagattggcctgttatttgatttcctcctttgggaaaagaattatgtagataccacatggagacagggaaacaattgtggtaaaactgtggatcctgttgctatttgcccagtgagaaaacagattctggtatttgatttggtttttctctttgtttccagaatggatgaaagtccatgaacctcctaagttataatttaaatttgtttggggcaaggtgattttatagtcgagacagagccctaggtccttcctgccccatcactcacttacgacatcacttccattgtgtgcatgtttgttatagaggaggttttaggctacaatatttgtttaacctccctaagaactttcaaggcatctgtcctgaaagctgttaatttatggtctagcagatttatattatatgcagataataattaactggggataaaagaatggcaaggggtgacacaaagtagcaaactgaatacttctccaatagcaaccccaagctacctcctcaccctgcatcttggagggaggcaggaaatttcttttgaaataaagtgctggagctgaattctgcattatttatcgttgctgctgaaaccacctataaaagacttgctggctaatgtgcattgtcatataatgtacactgtcacatctttacagtcttgtatgttatagaatacaaaataagttgatggttttgtttggtgtgagctttttgtttgtttgtttagttttgccttcataggttatatgccaagatagtatttgataagtcaatgacatttggatgttttcttcaaagaattttatttgacccagatttcttataaagttatcttacattaaggatgtcattttcatcagaccttctttctacatattattcatgaagcataatgttgcatttctccaaattttatgcctgaaagggtagtgttgcttcctaaggtatcatgttgtctttgtgctttgtccatctcttccgtggcgaagctttatatctgttcctaaaacagttaatcctgtgaaataaatattgaacataatccagaagaatctctctgtttcccttggggaatgccatatttaattcaccagcagtaatcctttaataactggcagagcactttattcttctggtgagctccctgaatatttatttttctgattataaattttctatattagtagcattttttaattattacttcttcactatagagcatttacttttagtctctagatgtatattttggaatgctgtacttggcataacatagattaaaatcataatgcatgactaaaaactccttggatttatttcccattttaaaatttttagcggtaagttcagatttataatctttctctagacttccatggtctgaatgttgcctgctgaagtagcaacctaaaaagtatcccctgcttatgcttgtccagttggccctccatgtccataggcttcgcatctgtgattcagcccactgtgggtcaaaaatatttggggaaaaaaatggatggttgcgcctttgctgaacatgtacaaacttttttttgtcattaaacaatatagtataacaactatttacaaagcatttacattgtattagctattataggtaatctagagatgatttaaagtgtatggtaggatgtgcataggttatatgcaaatactacaccattttctataagggacttgaacatcatggactttagtatcctagggggttcttggaacccatcacccataggggcaccataggacaactatagtaccgtgtttatttcctattaattcaggttccgtttagagtctaaaactaaaacctaatcatttagtcacagtgtaaaaacaaatggaaataacagctcaaatcttcaaaatattactatagcattatgtttaaaataatctacaacaaaaatgtaccattttcaagcagtactacattaggagcccttttatagaaaataatttcttctttacccccgttccagtgtgaatctagtattctgttaacatttgtgtggcatttggagtttgtcatccccattgaagggagagccttctcagacatgaagcaagggaaacatactgaatagttttacacaaatttgatctggcttccatttgtccccctcatttcccaaatgtttaaatgtattggatttggattctcaatgtataagttgccttatctgttaatgtctatcttctgtctctttaattttgtatatctgctgttttgcttttggatacattttctaattagaagtcacatgataaatataatcagtatagtaataataccataatgtgcacatactcaataaataaatgactgcattgttgtaaa

SEQ ID NO: 39 - DNA, 2835 bp, Homo sapiens
  source 1..2835: mol_type = genomic DNA
ctcgcccgtctgggcgtgggcgtggccggcgtggctgctcgggaccacccgaacccgcggccatggccccggccgccgccagccccccggaggtgatccgcgcggcgcagaaggacgagtactaccgcggtgggctgcggagcgcggcgggcggcgccctgcacagcctggcgggtgcgaggaagtggctggagtggaggaaggaggttgagctgctctcagatgtggcctactttggcctcaccacacttgcaggctaccagaccctgggggaggagtacgtcagcatcatccaggtggacccatcgcggatacatgtgccctcctcgctgcgccgtggcgtgctggtgacactgcatgccgtcctgccctacctgctggacaaggccctgctccccctggagcaggagctgcaggctgaccccgacagtgggcgacccttgcaggggagcctggggccaggtgggcgtggctgctcaggggcgcggcgctggatgcgtcaccacacggccaccctgactgagcagcagaggagggcgctgctgcgggcggtcttcgtcctcagacagggcctcgcctgcctccagcggctacatgttgcctggttttacatccacggtgtcttctaccacctggccaagaggctcacggggatcacgtacctccgtgtccgcagcctgcccggagaggacctgagggcccgtgttagctacaggctgctgggggtcatctcactgctgcacctggtgctgtccatggggctgcagctgtacggtttcaggcagcggcagcgagccaggaaggagtggaggctgcaccgcggcctgtctcaccgcagggcctccttggaggagagagccgtttccagaaaccccctgtgcaccctgtgcctggaggagcgcaggcacccaacagccacgccctgcggccacctgttctgctgggagtgcatcaccgcgtggtgcagcagcaaggcggagtgtcccctctgccgggagaagttccctccccagaagctcatctaccttcggcactaccgctgagccggcgcccgggtgggcctggacacagatgacctctacgggagtctgaacgccaagatttagtctcaggattaaccttgcttgcacagaagttagaacactctcagttttttgtcatgtaagatactaacctagccaccctgggagagaacagaaagctgtccctggctgcgctttctcagccctgggaggggcgcctgaacccagaacatttccctaaccccaacctggtaggactcagccacttcttcaggaatttcacttatttggacgggattttaggtttccctcccttccccaaaccatacagttgagaagtaattcagaagtaggccagaagacactttattcgtttatattgtgagaaaacagccccatcaggcttgtgttaaggcaatggactgaatgagtgcgtgctgggtggggtggggcacggaggctggcgggttgcttcagccagtgcagtgagaacagcagccccacggccccatgggaggcggcgctgctctccccgagggcggctgggcagagcacatcccccaggacttgatgaccacacggggcagagagaaaccaaccaaggccagcacctccgtcggaagcatttggcacacacaccttcaatacacgtcaaggtcgcttccagttttagaaaacagaaatctgcatctcagcctgagacgcacagagaggtctcttcctgacccagacgcactcacgagccaggtcctgggggtatgggggctgccaggggcgcccgagccctctcctggggggcctgctgggcaggcgacctgctgacccacggtcactgctgtgttcagcccctcagctcggccccagcctatttcccgcctccatttgatgtttccaggttttcaaaactgcatttaacctgcgccagagagttcaccgtaggcatctttaataaactaactccagcaaaatgtgggtacgttactaacacaagatggaacttgagatttcctgaagcgcagtgttaaaatgcctcgagggcgtgtgccacatttccctgctgacgtaaaacacctggccagcaggaccggcttccggcaaagacccgcgcaaaccatgtcttgctgtttgacttacttaggaatgaggatgaccctgtattcctgggttagtcaaggtcagcttgccagagacggaggagggcgggggggccgtgaccctctagactgggaggaccaccaggagaggcactgggggctgcggccgggtgggcagatgccgccgtggctccatccatcagcacccagtcctttgcttcactgtgctctggccaagcgcgtgcgacttcctgtcacaccttaggcctgagcgcttattaagaaaacagtatgaaaaatgacaaacactcctgtccacagtctgagaatcccaagtgtctcaacacggtaccagaagtaccacctgggagcagcactggctatcttgggtcagggacacacaagcttgaacgtggcctcttagaggagctcgatgtcagatatgtaaacactggtacaagaaaatccagacactaagcattcaccgtgtcaattctgagacaggaaacaccgcccagagtgaggagcctgcgcagcccggggcccaggaggagaggccttcactcgcatgtcagtgtctcatctgggaccacagctgtgatgttctgctgtgcaatatgggtcacctttcgaaaaggccaaacaaaaggtgggctttcctgtgtccagcgactgccatgttttaggtaaagactttagtaaaggtggaggtaaaatca

SEQ ID NO: 40 - DNA, 1743 bp, Homo sapiens
  source 1..1743: mol_type = genomic DNA
ataccagctctgagatttccagctcagcgatgcccccaggtccctgggagagctgcttctgggtggggggcctcattttgtggctcagcgttggaagttcaggggatgcacctcctaccccacagccaaagtgcgctgacttccagagcgccaacctttttgaaggcaccgatctcaaagtccagtttctcctctttgtcccttcgaatcctagctgtgggcagctagtagaaggaagcagtgacctccaaaactctgggttcaatgccactctgggaaccaaactaattatccatggattcagggttttaggaacaaagccttcctggattgacacatttattagaacccttctgcgtgcaacgaatgctaatgtgattgccgtggactggatttatgggtctacaggagtctacttctcagctgtgaaaaatgtgattaagttgagcctcgagatctcccttttcctcaataaactcctggtgctgggtgtgtcggaatcctcaatccacatcattggtgttagcctgggggcccacgttgggggcatggtgggacagctcttcggaggccagctgggacagatcacaggcctggaccccgctggacctgagtacaccagggccagtgtggaagagcgcttggatgctggagatgccctcttcgtggaagccatccacacagacaccgacaatttgggtattcggattcccgttggacatgtggactacttcgtcaacggaggccaagaccaacctggctgccccaccttcttttacgcaggttatagttatctgatctgtgatcacatgagggctgtgcacctctacatcagcgccctggagaattcctgtccactgatggcctttccctgtgccagctacaaggccttccttgctggacgctgtctggattgctttaacccttttctgctttcctgcccaaggataggactggtggaacaaggtggtgtcaagatagagccgctccccaaggaagtgaaagtctacctcctgactacttccagtgctccgtactgcatgcatcacagcctcgtggagtttcacttgaaggaactgagaaacaaggacaccaacatcgaggttaccttccttagcagtaacatcacctcttcatctaagatcaccatacctaagcagcaacgctatgggaaaggaatcatagcccatgccaccccacaatgccagataaaccaagtgaaattcaagtttcagtcttccaaccgagtttggaaaaaagaccggactaccattattgggaagttctgcactgcccttttgcctgtcaatgacagagaaaagatggtctgcttacctgaaccagtgaacttacaagcaagtgtgactgtttcctgtgacctgaagatagcctgtgtgtagtttaacctgggcaggacacatctccctgcattttttttttttttttgagagagaggtgtgatgagggatgtgtgtgtgcagcttattgtagaccattactactaaggagaaaagcaaagctctttcttattttcctcataatcagctaccctggaggggagggagaactcattttacagaacttggtttcctttgccgatcttatgtacatacccattttagctttcccatgcatacttaactgcacttgctttatctccttgggcattcgtacttaggattcaatagaaacatgtacagggtaaacaattttttaaaaataaaacttcatggagtatctgaa

SEQ ID NO: 41 - DNA, 1823 bp, Homo sapiens
  source 1..1823: mol_type = genomic DNA
agtgtattcagagaacacggtgaaacaaggaaaaccggcctgactggggggtgaattcagcagggagtaaatctgatcggcatcaggtctgcggaaaggagctggagactaagctgaaactgctgctcagctcccaagatggtgccacccaaattgcatgtgcttttctgcctctgcggctgcctggctgtggtttatccttttgactggcaatacataaatcctgttgcccatatgaaatcatcagcatgggtcaacaaaatacaagtactgatggctgctgcaagctttggccaaactaaaatcccccggggaaatgggccttattccgttggttgtacagacttaatgtttgatcacactaataagggcaccttcttgcgtttatattatccatcccaagataatgatcgccttgacaccctttggatcccaaataaagaatatttttggggtcttagcaaatttcttggaacacactggcttatgggcaacattttgaggttactctttggttcaatgacaactcctgcaaactggaattcccctctgaggcctggtgaaaaatatccacttgttgttttttctcatggtcttggggcattcaggacactttattctgctattggcattgacctggcatctcatgggtttatagttgctgctgtagaacacagagatagatctgcatctgcaacttactatttcaaggaccaatctgctgcagaaataggggacaagtcttggctctaccttagaaccctgaaacaagaggaggagacacatatacgaaatgagcaggtacggcaaagagcaaaagaatgttcccaagctctcagtctgattcttgacattgatcatggaaagccagtgaagaatgcattagatttaaagtttgatatggaacaactgaaggactctattgatagggaaaaaatagcagtaattggacattcttttggtggagcaacggttattcagactcttagtgaagatcagagattcagatgtggtattgccctggatgcatggatgtttccactgggtgatgaagtatattccagaattcctcagcccctcttttttatcaactctgaatatttccaatatcctgctaatatcataaaaatgaaaaaatgctactcacctgataaagaaagaaagatgattacaatcaggggttcagtccaccagaattttgctgacttcacttttgcaactggcaaaataattggacacatgctcaaattaaagggagacatagattcaaatgtagctattgatcttagcaacaaagcttcattagcattcttacaaaagcatttaggacttcataaagattttgatcagtgggactgcttgattgaaggagatgatgagaatcttattccagggaccaacattaacacaaccaatcaacacatcatgttacagaactcttcaggaatagagaaatacaattaggattaaaataggttttttaaaagtcttgtttcaaaactgtctaaaattatgtgtgtgtgtgtgtgtgtgtgtgtgtgtgtgagagagagagagagagagagagagagagagagagagaattttaatgtattttcccaaaggactcatattttaaaatgtaggctatactgtaatcgtgattgaagcttggactaagaattttttccctttagatgtaaagaaagaatacagtatacaatattcatatcagcctaaattttaattttaaagatgattccttttcagtgtcgaagttaaaaactgtttttacattactttgacagacaagtagattaaaacaggcaaaatcccagtgaaaa

SEQ ID NO: 42 - DNA, 2343 bp, Homo sapiens
  source 1..2343: mol_type = genomic DNA
tttctttttgtttgctgccttccgtagaagatgtggcttgctcatgcttgacttctgccatggttgtgaggcctccccagccatgtggaactgttttcaggtgctggttccatggctcttcctgagccgaaaataaggaaactccatagaccttgtccactggaactcgttcccatctaccctccactctatccagccccatgaggacaaggaacatgattggttttgctcactgccgtatcttccgtacctagtacgtaacaggacatcaataaatattagttgaatggaagattaaatcaacaaaatgggtgatggatctctgcagtaagtggaagagttcttcatggcccccaaggttatatccatctagaacttcagcacgtaatttcatctggaaatagtgcctttgcggatataagttaggtaaaactgaagatgagatcatactggattaggatgggatctaaatccaatgaaaatgtcttcataaaaaacaggaaagaacccatagaaacacaaggaagaaggtcatgtgaagatggaggcagagattggagggatgcagccaccggcccaggaatgccagcagccacccagaagctggaaggaaatgagggattctctcctagaacctttagagagaacatggtcctgtgaacagcttgattttggacttgcccatagcttgtatactcttactttggatacaattttatccaaacttggctaaacagtttctcagcctatggaaaatttaaaatggagaagattcaactcgattcttacagattcaaagcaagaaaatgatgggaacataggaggagaccaagaaagcctataaaaagcaaaaatatgaagtgaacattgtggtagctttaagatgtttagtgtagctgcaggcaccctatacacatgaaaacccccaaggggaatccccatatcacagtgtagtgtgatatttgacattcgtgatcatctagagatgtacagaaaaggtgaatctgtgttctgtatattctgcctaaggcaaagaaatgtttagctctctttaaaatagttccataattttttctaaaaagctttgcttgaaaactgtaagcttcccatatctggagcatttcactttaaatatttggataaatatgttatcttcttacttggacatttcatgtgtttagggattgtcttctaaattcttcctaattcatatagctgctaacacttcccgcagagctaaaccattacagatatgaaataaagacctattgatttgacttacttttacttgtaaaaccttctgagtgttataacctcatttaatctttcagcatttacagtttcaagagtttgtgtcacaattagaagaattcagctgcacctccaagtgacagcagttgctctggttggtgggtgatctcaggaggcttgagaattgctttgtctgtgagggaagtaagacattttcagagccccactttagaaggtgtgaactggctagataatgaaccccagggctaacgctgctatagcagtgggaaggaggtgatgggttttcagtttggacctcaaacatcaataccctcctggtacggggaggaacagagtccctcctttacttctccattagaaagaatgagatggcaagacaatgaaacaggcaaagtgaacagagatgcaagacaaaattcaggtgagagagccagagcatcactcagccattcctgacatgtaaaacaggcaactagaaatttgcagaaaggaagcgaagtctccataaagatgtttttaaagtgagcttgaagtatttggagacaattcagtgttacataaaatctgcaaatctctggataaagaagcagagatcccagcatgggacaaatggagcctcaaaagtgggaagaagacagagaagaccagggcagaatgcatctcttcctttctcttggctttcctggataaggactgcatcattcctgtggaaggacaggccatcagctccgaaacactgtatgtattttccagtatatactgctagctgtgtgatgttgggaaaatttgttaccctgtctaacccccacttccctcatctgtaaaatggaaataatgatagtacctacctatctcataggtggcaactacaaggggcagcacactcagggaattaaggaagtttcagtgaacatcaactttatgaacacagtgttcataaaggcaggtcagtgcagtggtttgggagccaggagaagcacgtgggccggagtgtgcctgcaggagacaaggtcagagatgttgctaataatggagaataaaggatgcattctcattactgaaaaaaaaaaa

SEQ ID NO: 43 - DNA, 240 bp, Homo sapiens
  source 1..240: mol_type = genomic DNA
atgaaggtaacaggcatctttcttctcagtgccttggccctgttgagtctatctggtaacactggagctgactccctgggaagagaggccaaatgttacaatgaacttaatggatgcaccaagatatatgaccctgtctgtgggactgatggaaatacttatcccaatgaatgcgtgttatgttttgaaaatcggaaacgccagacttctatcctcattcaaaaatctgggccttgctga

SEQ ID NO: 44 - DNA, 1700 bp, Homo sapiens
  source 1..1700: mol_type = genomic DNA
aggtcaatgatagcatctgcctagagtcaaacctccgtgcttctcagacagtgccttttcaccatgagtgggtgcccatttttaggaaacaactttggatatacttttaaaaaactccccgtagaaggcagcgaagaagacaaatcacaaactggtgtgaatagagccagcaaaggaggtcttatctatgggaactacctgcatttggaaaaagttttgaatgcacaagaactgcaaagtgaaacaaaaggaaataaaatccatgatgaacatctttttatcataactcatcaagcttatgaactctggtttaagcaaatcctctgggagttggattctgttcgagagatctttcagaatggccatgtcagagatgaaaggaacatgcttaaggttgtttctcggatgcaccgagtgtcagtgatcctgaaactgctggtgcagcagttttccattctggagacgatgacagccttggacttcaatgacttcagagagtacttatctccagcatcaggcttccagagtttgcaattccgactattagaaaacaagataggtgttcttcagaacatgagagtcccttataacagaagacattatcgtgataacttcaaaggagaagaaaatgaactgctacttaaatctgagcaggaaaagacacttctggaattagtggaggcatggctggaaagaactccaggtttagagccacatggatttaacttctggggaaagcttgaaaaaaatatcaccagaggcctggaagaggaattcataaggattcaggctaaagaagagtctgaagaaaaagaggaacaggtggctgaatttcagaagcaaaaagaggtgctactgtccttatttgatgagaaacgtcatgaacatctccttagtaaaggtgaaagacggctgtcatacagagcacttcagggagcattgatgatatatttttacagggaagagcctaggttccaggtgccttttcagttgctgacttctcttatggacatagattcactgatgaccaaatggagatataaccatgtgtgcatggtgcacagaatgctgggcagcaaagctggcaccggtggttcctcaggctatcactacctgcgatcaactgtgagtgataggtacaaggtatttgtagatttatttaatctttcaacatacctgattccccgacactggataccgaagatgaacccaaccattcacaaatttctatatacagcagaatactgtgatagctcctacttcagcagtgatgaatcagattaaaatcgtctgcaaaatctatgaagaatactggtttcacagcctattttttattttctatggattttcataaatacagtttgaatatatgtatgcatatattgttcagcaccacgatgctctgatttaattctagaaacaatttgattacctcttgtttgtgacaagactaagcattaagatgagaaagaatacatttaaatagtaacattgtacatagggtgttttcctattaaaaattcagtttcccctgagacttaatgtaaccacttaatgtaatcactatctcattgtttcatctttataaacttgtaaacttcatctatttcaaatattttatgcagtacattatattattctgtacaaaggctttcaaacaaaatttttaaaataataaagtattaatctttctccctgta

SEQ ID NO: 45 - DNA, 1431 bp, Homo sapiens
  source 1..1431: mol_type = genomic DNA
ggcttactgcgggacggccttggagagtactcgggttcgtgaacttcccggaggcgcaatgagctgcattaacctgcccactgtgctgcctggctcccccagcaagacccgggggcagatccaggtgattctcgggccgatgttctcaggaaaaagcacagagttgatgagacgcgtccgtcgcttccagattgctcagtacaagtgcctggtgatcaagtatgccaaagacactcgctacagcagcagcttctgcacacatgaccggaacaccatggaggcactgcccgcctgcctgctccgagacgtggcccaggaggccctgggcgtggctgtcataggcatcgacgaggggcagtttttccctgacatcgtggagttctgcgaggccatggccaacgccgggaagaccgtaattgtggctgcactggatgggaccttccagaggaagccatttggggccatcctgaacctggtgccgctggccgagagcgtggtgaagctgacggcggtgtgcatggagtgcttccgggaagccgcctataccaagaggctcggcacagagaaggaggtcgaggtgattgggggagcagacaagtaccactccgtgtgtcggctctgctacttcaagaaggcctcaggccagcctgccgggccggacaacaaagagaactgcccagtgccaggaaagccaggggaagccgtggctgccaggaagctctttgccccacagcagattctgcaatgcagccctgccaactgagggacctgcgagggccgcccgctcccttcctgccactgccgcctactggacgctgccctgcatgctgcccagccactccaggaggaagtcgggaggcgtggagggtgaccacaccttggccttctgggaactctcctttgtgtggctgccccacctgccgcatgctccctcctctcctacccactggtctgcttaaagcttccctctcagctgctgggacgatcgcccaggctggagctggccccgcttggtggcctgggatctggcacactccctctccttggggtgagggacagagccccacgctgttgacatcagcctgcttcttcccctctgcggctttcactgctgagtttctgttctccctgggaagcctgtgccagcacctttgagccttggcccacactgaggcttaggcctctctgcctgggatgggctcccaccctcccctgaggatggcctggattcacgccctcttgtttccttttgggctcaaagcccttcctacctctggtgatggtttccacaggaacaacagcatctttcaccaagatgggtggcaccaaccttgctgggacttggatcccaggggcttatctcttcaagtgtggagagggcagggtccacgcctctgctgtagcttatgaaattaactaattgaaaattca

SEQ ID NO: 46 - DNA, 2798 bp, Homo sapiens
  source 1..2798: mol_type = genomic DNA
agagggatgcgggcggcagagctcgagaggcggctgccgggctgcggggcgccttgactctccctccaccctgcctcctcgggctccactcgtctgcccctggactcccgtctcctcctgtcctccggcttcccagagctccctccttatggcagcagcttcccgcgtctccggcgcagcttctcagcggacgaccctctcgctccggggctgagcccagtccctggatgttgctgaaactctcgagatcatgcgcgggtttggctgctgcttccccgccgggtgccactgccaccgccgccgcctctgctgccgccgtccgcgggatgctcagtagcccgctgcccggcccccgcgatcctgtgttcctcggaagccgtttgctgctgcagagttgcacgaactagtcatggtgctgtgggagtccccgcggcagtgcagcagctggacactttgcgagggcttttgctggctgctgctgctgcccgtcatgctactcatcgtagcccgcccggtgaagctcgctgctttccctacctccttaagtgactgccaaacgcccaccggctggaattgctctggttatgatgacagagaaaatgatctcttcctctgtgacaccaacacctgtaaatttgatggggaatgtttaagaattggagacactgtgacttgcgtctgtcagttcaagtgcaacaatgactatgtgcctgtgtgtggctccaatggggagagctaccagaatgagtgttacctgcgacaggctgcatgcaaacagcagagtgagatacttgtggtgtcagaaggatcatgtgccacagatgcaggatcaggatctggagatggagtccatgaaggctctggagaaactagtcaaaaggagacatccacctgtgatatttgccagtttggtgcagaatgtgacgaagatgccgaggatgtctggtgtgtgtgtaatattgactgttctcaaaccaacttcaatcccctctgcgcttctgatgggaaatcttatgataatgcatgccaaatcaaagaagcatcgtgtcagaaacaggagaaaattgaagtcatgtctttgggtcgatgtcaagataacacaactacaactactaagtctgaagatgggcattatgcaagaacagattatgcagagaatgctaacaaattagaagaaagtgccagagaacaccacataccttgtccggaacattacaatggcttctgcatgcatgggaagtgtgagcattctatcaatatgcaggagccatcttgcaggtgtgatgctggttatactggacaacactgtgaaaaaaaggactacagtgttctatacgttgttcccggtcctgtacgatttcagtatgtcttaatcgcagctgtgattggaacaattcagattgctgtcatctgtgtggtggtcctctgcatcacaaggaaatgccccagaagcaacagaattcacagacagaagcaaaatacagggcactacagttcagacaatacaacaagagcgtccacgaggttaatctaaagggagcatgtttcacagtggctggactaccgagagcttggactacacaatacagtattatagacaaaagaataagacaagagatctacacatgttgccttgcatttgtggtaatctacaccaatgaaaacatgtactacagctatatttgattatgtatggatatatttgaaatagtatacattgtcttgatgttttttctgtaatgtaaataaactatttatatcacacaatatagttttttctttcccatgtatttgttatatataataaatactcagtgatgagaaaaaattggcattcttaaatttgcggtatctcataactgtaaatataatcagactagtacaatctgtacagctaccaatatttcatgtttcttctcatcttgagacagcacattagttcgtacaggactcagtggctaggttttgaatgattccaagatcaagggaaatgatggttattggaaaagagaaaaaataatttactttatatcgagtgaggataaaatatttccgatctttgaatcatctctatttcatcaactttcttccctggtcttccattttcatccctagagcagaaaaatctctggcatataaactaaataaaagaagaaggggagggaaagtgttttataactcataaaggagagggaaagaaaatattggtttttattggggaagtagcttagaatcccccagttaagtgcatatatctgaacttactgaacaagttacatactaggtatacacagagtggcaaaatatattccatttaggtgggtggaattaccaggggaaaaatgtaataacaccactagatgtgaaacaccaaaatcgtgaattctcaaaagcaccatacaatatgtatagtatatagttctttgaaaagaagttagaatcacaaccaataccccatgaatagctttgtggctaatgcagcaccataatttgtaatggaactaagatgatgatgacgatatttcatgaaaacagagagatgttttgagcatatttatgtggtgaggtaagaaagaaaattaatcctatagcatctgaaagacctcactgggaagttggtatggatttttgtttgatttgtgcatacaaataggtatcacaacttgatctggaaaaaataagctgtgaaaattctcaaggaataagatgaaaataaatcaattattatcatttagctctgcaaagctttccatggctaacacagtaaatttaaataaactctctttgtctccttcaaa

SEQ ID NO: 47 - DNA, 1936 bp, Homo sapiens
  source 1..1936: mol_type = genomic DNA
gcagtcggaggcggccggctgggcgtgcgctcgctccccgaagccggggctgggccggagccgggcgagggctgggagctgggccgggtccggggacagcgggcgaggggcagctgccggagccgggcagccaggccgctcagggcaggggacagctggcgccggttctgcggtctccggggcccagatgtgaggcggcggcgcccccggcccgagagcgcacgatgggggccccgctcgccgtagcgctgggcgccctccactacctggcacttttcctgcaactcggcggcgccacgcggcccgccggccacgcgccctgggacaaccacgtctccggccacgccctgttcacagagacaccccatgacatgacagcacggacgggcgaggacgtggagatggcctgctccttccgcggcagcggctccccctcctactcgctggagatccagtggtggtatgtacggagccaccgggactggaccgacaagcaggcgtgggcctcgaaccagctaaaagcatctcagcaggaagacgcagggaaggaggcaaccaaaataagtgtggtcaaggtggtgggcagcaacatctcccacaagctgcgcctgtcccgggtgaagcccacggacgaaggcacctacgagtgccgcgtcatcgacttcagcgacggcaaggcccggcaccacaaggtcaaggcctacctgcgggtgcagccaggggagaactccgtcctgcatctgcccgaagcccctcccgccgcgcccgccccgccgccccccaagccaggcaaggagctgaggaagcgctcggtggaccaggaggcctgcagcctctagactgatgcccctgcccccgcccatccgcccccacgctgtacagagtgcatgaggagccgccggaccaccggggaccgactgcctgcgtccagccgcgccccatccccgaggccgcctgtggccaccatgtcggccctctttccaccaccccttgctcagcatgtaagccccacccacccctgccctttcagacccctgcggtgacctggctcggagaaggtggccctgggcaccaaggggccaaccgccctgaacactggggcagggaccatgctggggcccggggccacccccttcctgtcaccagcttctgtggagtccagtgttttgctttgcttgcttgtcccccatcctgtcctgagccggggccccccagcctcgcctccctcctcctaccatccctcacttggacctgggggtgtggacagtgacccctccctgaatatggacttgaatcttctgagcagaactagggcctctcccctggtgaagacccagggaacccaggagggcccttctggggcagtggctctgcagggtcactcatggaggcctaggggaacagcgagatgccccaccacctcctggcgagtccttcctgttcagctccctgtgcgaccctccagggatgcaggggatccaggattctctgccctgtcacacggcgagtcagaagggaggggcctttccctcggacccatggccccaggcagagttttgcaccagcaggacccctttgagggccttcaaggctctcccaggagtccccctctgccggccccccaatgccccagctccctcttgggtcctgtgccaagtccgccccagggcctggggctgttgggagccaagggccccctggtactcagttccctcacgattcccgatcacgggcacacctgccccctggttatttgtaaatatttctattggacccaattctcctcggaattggctggcacctctggctgccgcagctcagtgatgacgtgggggaggtgggagaggccgagggctttgcctaggggtgggttgccctgtatacatgatccagtctgtgactaccagccaacctgaataaagcggttttaaaaaaa

SEQ ID NO: 48 - DNA, 2829 bp, Homo sapiens
  source 1..2829: mol_type = genomic DNA
aaactcaccacctggccgtggacacctgtgtcagcatgtgggacctggttctctccatcgccttgtctgtggggtgcactggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgctccgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtgggggtgattctgggggtccacttgtctgtaatggtgtgcttcaaggtatcacatcatggggccctgagccatgtgccctgcctgaaaagcctgctgtgtacaccaaggtggtgcattaccggaagtggatcaaggacaccatcgcagccaacccctgagtgcccctgtcccacccctacctctagtaaatttaagtccacctcacgttctggcatcacttggcctttctggatgctggacacctgaagcttggaactcacctggccgaagctcgagcctcctgagtcctactgacctgtgctttctggtgtggagtccagggctgctaggaaaaggaatgggcagacacaggtgtatgccaatgtttctgaaatgggtataatttcgtcctctccttcggaacactggctgtctctgaagacttctcgctcagtttcagtgaggacacacacaaagacgtgggtgaccatgttgtttgtggggtgcagagatgggaggggtggggcccaccctggaagagtggacagtgacacaaggtggacactctctacagatcactgaggataagctggagccacaatgcatgaggcacacacacagcaaggatgacgctgtaaacatagcccacgctgtcctgggggcactgggaagcctagataaggccgtgagcagaaagaaggggaggatcctcctatgttgttgaaggagggactagggggagaaactgaaagctgattaattacaggaggtttgttcaggtcccccaaaccaccgtcagatttgatgatttcctagcaggacttacagaaataaagagctatcatgctgtggtttattatggtttgttacattgataggatacatactgaaatcagcaaacaaaacagatgtatagattagagtgtggagaaaacagaggaaaacttgcagttacgaagactggcaacttggctttactaagttttcagactggcaggaagtcaaacctattaggctgaggaccttgtggagtgtagctgatccagctgatagaggaactagccaggtgggggcctttccctttggatggggggcatatctgacagttattctctccaagtggagacttacggacagcatataattctccctgcaaggatgtatgataatatgtacaaagtaattccaactgaggaagctcacctgatccttagtgtccagggtttttactgggggtctgtaggacgagtatggagtacttgaataattgacctgaagtcctcagacctgaggttccctagagttcaaacagatacagcatggtccagagtcccagatgtacaaaaacagggattcatcacaaatcccatctttagcatgaagggtctggcatggcccaaggccccaagtatatcaaggcacttgggcagaacatgccaaggaatcaaatgtcatctcccaggagttattcaagggtgagccctttacttgggatgtacaggctttgagcagtgcagggctgctgagtcaaccttttattgtacaggggatgagggaaagggagaggatgaggaagcccccctggggatttggtttggtcttgtgatcaggtggtctatggggctatccctacaaagaagaatccagaaataggggcacattgaggaatgatactgagcccaaagagcattcaatcattgttttatttgccttcttttcacaccattggtgagggagggattaccaccctggggttatgaagatggttgaacaccccacacatagcaccggagatatgagatcaacagtttcttagccatagagattcacagcccagagcaggaggacgctgcacaccatgcaggatgacatgggggatgcgctcgggattggtgtgaagaagcaaggactgttagaggcaggctttatagtaacaagacggtggggcaaactctgatttccgtgggggaatgtcatggtcttgctttactaagttttgagactggcaggtagtgaaactcattaggctgagaaccttgtggaatgcagctgacccagctgatagaggaagtagccaggtgggagcctttcccagtgggtgtgggacatatctggcaagattttgtggcactcctggttacagatactggggcagcaaataaaactgaatcttgttttcagacctta

SEQ ID NO: 49 - DNA, 745 bp, Homo sapiens
  source 1..745: mol_type = genomic DNA
agtgtcccggccgcgcaggacttgacatgctgcccgactgcctgtcggccgagggcgagctgcgctgccgccggctgctggcaggggccacggcccggctccgcgcgcggcccgcgtcggccgcggtgctcgtgccgctctgctcagtgcgtggggtcccggcgctgctgtacacgctgcggtccagccgcctgaccgggaggcacaagggcgacgtcagtttcccaggcggcaagtgcgacccggctgaccaagatgtggtgcacacggccctgcgggaaacccgggaggagctgggcctggcagtgcccgaggagcacgtgtggggcctgctgcggcctgtgtatgatccgcaaaaggccaccgtggtgccagtgcttgctggtgtaggcccactggatccccagagcctcaggcccaactcggaggaggtagatgaggtgtttgcactgccgctggcccacctgctgcagacgcagaatcagggctatacccacttctgccggggtggccacttccgctacacactacccgtcttcctgcatggaccacaccgggtctggggcctcacagctgtcatcactgagtttgccctgcagctgctggcacctggtacctaccagccccgcctggccggcctgacctgctcaggggctgagggtctggcccgccctaagcagcccctggcttcaccctgtcaggccagctccactccaggactgaataaaggtctttgacagctcta

SEQ ID NO: 50 - DNA, 1773 bp, Homo sapiens
  source 1..1773: mol_type = genomic DNA
ccctctggctgagacctcggctccggaatcactgcagcccccctcgccctgagccagagcaccccgggtcccgccagcccctcacactcccagcaaaatgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaagtgaagcgcgggcgcccgcggcgcgaccctccccggcggtgccgcgctccgaaccccgggcccgggcccccgccccgcccccgccccgcgcgccggtccggcgccccgcacccccgccaggcgcatgtctgcacctccgcttgccagaggccctcggtcagcgactggatgctcgccatcaaggtccagtggaagttcttcaagaggaaaggcgcccccgccccaggcttccgcgcccagcgctcgccacgctcagtgcccgttttaccaataaactgagcgacccca

SEQ ID NO: 51 - DNA, 1910 bp, Homo sapiens
  source 1..1910: mol_type = genomic DNA
cttcatctcgcggctgtctgacttcctcccagcacattcctgcactctgccgtgtccacactgccccacagacccagtcctccaagcctgctgccagctccctgcaagcccctcaggttgggccttgccacggtgccagcaggcagccctgggctgggggtaggggactccctacaggcacgcagccctgagacctcagagggccaccccttgagggtggccaggcccccagtggccaacctgagtgctgcctctgccaccagccctgctggcccctggttccgctggccccccagatgcctggctgagacacgccagtggcctcagctgcccacacctcttcccggcccctgaagttggcactgcagcagacagctccctgggcaccaggcagctaacagacacagccgccagcccaaacagcagcggcatgggcagcgccagcccgggtctgagcagcgtatcccccagccacctcctgctgccccccgacacggtgtcgcggacaggcttggagaaggcggcagcgggggcagtgggtctcgagagacgggactggagtcccagtccacccgccacgcccgagcagggcctgtccgccttctacctctcctactttgacatgctgtaccctgaggacagcagctgggcagccaaggcccctggggccagcagtcgggaggagccacctgaggagcctgagcagtgcccggtcattgacagccaagccccagcgggcagcctggacttggtgcccggcgggctgaccttggaggagcactcgctggagcaggtgcagtccatggtggtgggcgaagtgctcaaggacatcgagacggcctgcaagctgctcaacatcaccgcagatcccatggactggagccccagcaatgtgcagaagtggctcctgtggacagagcaccaataccggctgccccccatgggcaaggccttccaggagctggcgggcaaggagctgtgcgccatgtcggaggagcagttccgccagcgctcgcccctgggtggggatgtgctgcacgcccacctggacatctggaagtcagcggcctggatgaaagagcggacttcacctggggcgattcactactgtgcctcgaccagtgaggagagctggaccgacagcgaggtggactcatcatgctccgggcagcccatccacctgtggcagttcctcaaggagttgctactcaagccccacagctatggccgcttcattaggtggctcaacaaggagaagggcatcttcaaaattgaggactcagcccaggtggcccggctgtggggcatccgcaagaaccgtcccgccatgaactacgacaagctgagccgctccatccgccagtattacaagaagggcatcatccggaagccagacatctcccagcgcctcgtctaccagttcgtgcaccccatctgagtgcctggcccagggcctgaaacccgccctcaggggcctctctcctgcctgccctgcctcagccaggccctgagatgggggaaaacgggcagtctgctctgctgctctgaccttccagagcccaaggtcagggaggggcaaccaactgccccagggggatatgggtcctctggggccttcgggaccctggggcaggggtgcttcctcctcaggcccagctgctcccctggaggacagagggagacagggctgctccccaacacctgcctctgaccccagcatttccagagcagagcctacagaagggcagtgactcgacaaaggccacaggcagtccaggcctctctctgctccatccccctgcctcccattctgcaccacacctggcatggtgcagggagacatctgcacccctgagttgggcagccaggagtgcccccgggaatggataataaagatactagagaactga

SEQ ID NO: 52 - DNA, 1285 bp, Homo sapiens
  source 1..1285: mol_type = genomic DNA
gctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacca

SEQ ID NO: 53 - DNA, 2930 bp, Homo sapiens
  source 1..2930: mol_type = genomic DNA
gctgagtgctcagtggagagcggggagttgtgtccaccttgccgacgtcgctagccgtggggctgtcctgggaaggcggacggcgagcgcccggtgtccgcactcggccgcctgccgtgcccgtctgcgcccgtgtcatcctcactcgggacgcagggaccgtttttaaatcacaggggcgtgtgtcagcctgccctaggacttcatgtctatatatttccccattcactgccccgactatctgagatcggccaagatgactgaggtgatgatgaacacccagcccatggaggagatcggcctcagcccccgcaaggatggcctttcctaccagatcttcccagacccgtcagattttgaccgctgctgcaaactgaaggaccgtctgccctccatagtggtggaacccacagaaggggaggtggagagcggggagctccggtggccccctgaggagttcctggtccaggaggatgagcaagataactgcgaagagacagcgaaagaaaataaagagcagtagagtccctgtggactcccatgggtcataccagccagcatctgttcctgaactgtgtttttcccatcatgacggaagaagagagtgagccgcaattgttctgaaaatgtcaaacgaggcttctgttttgcacctgcagatcaccgagttggttttcttttcttttcttgcctttttttttttttgaaatttgccgagcagtggagccctctgacaatttgcaaggccctctgagaaaggaagctgcttagagccagggggttagtgggtgaggggagcgagtgctgtttttgagatcattatctgaactcaggcagcctagtagaggcagtggtgggattccaatgggtcttggtgggtgggaggtggggcatgtgcaaagcaagcaaggaacatttggggtaagaaaacaaacatgaggcaaaagaaaaaatacatgtttttaagaaaacattgagcagagaactgcagccaggatgcgctcagcagacattcactctggctgctgggacatcagaaaacaaagtcttcatctctctctccagtttcacccaccccaccctttgctttcatttcaggtgtgttggtctatatgacagggaggagagtaaaggagagcaggagcaattggctgcctgcaaagccagctggaggtgaagtgcaggaaaggaaaggtcaccccattctactccatggcctctctgctcccagctgtggtaggctcacatagccagtgtgatcggtttttaagaggcagtgcttttcagcttttctccctgatatatccattttgcttcccagcactttttaggagtagtgagagcacttcctgcccttgttggaagccccagggtggacactcagcacgaaggtctctcccttaactgctgcccttccaagacttgctcccgagatggagtgggcgtggtcttccaggctggcccttccttctcctcaccgccaccttccctgccccagccccagcagccatgggtacatgggtccccagctcacctatggattcccgccagtctgcccagctgcagtactcacgccccatgggggatcttggtctgtttttcttgtgggagcctagtggagagcagacgtggctttttatgtgtcttgttggggaggtgacttgcatggtggggacaaggctgtcgtggcaaccttgggatcgagtttgagactaaaggatgtcatgagatccctggcttctccccatgttgttcccggacaagggcagaagggaggcatggcaagggacctctgctgtccttactcaacagtggtcctcatccctccccacctcccactgcttcctgcaagggcaccagttgtatgagaaagttggcctttggacttaggatttcttattgtagctaagagccatctgaagcagcaggttgcaggacaaatgcttcagtccgccgagagcagtaccgtgtggccaagaggtggactcagagccttccttgagctaaactcggccaaccaaggcacgcagcatgtcccctcaggtctccagtcagtccaggttgaccctcagttctggacgtgtgtatatagctgtatttaatacctcaaggtcattgtggctctggggatgccggggcaggaggacgagggtgcgctgtggacacagcagtccgcggaattccgttctgggaagccaatggtcgccggcaccccttgcttcctccctctgttgtctgcctgtgtgacacacatcaatggcaataacttcttccaactcctcgcagaagtgggagaggccggcagcctgcaccgagaggggctttcctctctcttgctccccgcttcgttctgttttggctgcagagagtggttcatccatactctcattccctcgcctccccttgtggacgggggtcttgccttttcaattcctgtgttttggtgtcttcccttatctgctaccctgaatcacctgtcctggtcttgctgtgtgatgggaacatgcttgtaaactgcgtaacaaatctactttgtgtatgtgtctgtttatgggggtggtttattatttttgctggtccctagaccactttgtatgaccgtttgcagtctgagcaggccaggggctgacagctaatgtcaggaccctcagcggtggagcctgctggggggacccagctgctcttggacaagtggctgagctcctatctggcctcctctttttttttttttcaagtaatttgtgtgtatttctaactgattgtattgaaaaaattcctagtatttcagtaaaaatgcctgttgtgagatgaacctcctgtaacttctatctgttcttttttgaggctcagggagaaactagcatttttttttttccaaactactttttgtcactgtgacagttgtaaataaagtttgaaaatgctttcca

SEQ ID NO: 54 - DNA, 1746 bp, Homo sapiens
  source 1..1746: mol_type = genomic DNA
ggtgaggcagcctggcctagcaggccccacgccaccgcctctgcctccaggccgcccgctgctgcggggccaccatgctcctgcccaggcctggagactgacccgaccccggcactacctcgaggctccgcccccacctgctggaccccagggtcccaccctggcccaggaggtcagccagggaatcattaacaagaggcagtgacatggcgcagaaggagggtggccggactgtgccatgctgctccagacccaaggtggcagctctcactgcggggaccctgctacttctgacagccatcggggcggcatcctgggccattgtggctgttctcctcaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctctgaccggtggcttctcgctgcgcagcctccagggcccgaggtgatcccggtggtgggatccacgctgggcctaggatgggacgtttttcttcttgggcccggtccacaggtccaaggacaccctccctccagggtcctctcttccacagtggcgggcccactcagccccgagaccacccaacctcaccctcctgacccccatgtaaatattgttctgctgtctgggactcctgtctaggtgcccctgatgacgggatgctctttaaataataaagatggttttgattaa

SEQ ID NO: 55 - DNA, 5580 bp, Homo sapiens
  source 1..5580: mol_type = genomic DNA
agaagttgacggcgcagccgggcgcggggcgcggagtcggcggggcctcgcgggacgccgggcagtgcggagaccgcggcgctgaggacgcgggagccgggagcgcagccgcggggtggagttcagcctactctttcttagatgtgaaaggaaaggaagatcatttcatgccttgttgataaaggttcagacttctgctgattcataaccatttggctctgagctatgacaagagaggaaacaaaaagttaaacaagcaagcctgccataagtgagaagcaaacttccttgataacatgcttttgcgaagtgcaggaaaattaaatgtgggcaccaagaaagaggatggtgagagtacagcccccaccccccgtccaaaggtcttgcgttgtaaatgccaccaccattgtccagaagactcagtcaacaatatttgcagcacagacggatattgtttcacgatgatagaagaggatgactctgggttgcctgtggtcacttctggttgcctaggactagaaggctcagattttcagtgtcgggacactcccattcctcatcaaagaagatcaattgaatgctgcacagaaaggaacgaatgtaataaagacctacaccctacactgcctccattgaaaaacagagattttgttgatggacctatacaccacagggctttacttatatctgtgactgtctgtagtttgctcttggtccttatcatattattttgttacttccggtataaaagacaagaaaccagacctcgatacagcattgggttagaacaggatgaaacttacattcctcctggagaatccctgagagacttaattgagcagtctcagagctcaggaagtggatcaggcctccctctgctggtccaaaggactatagctaagcagattcagatggtgaaacagattggaaaaggtcgctatggggaagtttggatgggaaagtggcgtggcgaaaaggtagctgtgaaagtgttcttcaccacagaggaagccagctggttcagagagacagaaatatatcagacagtgttgatgaggcatgaaaacattttgggtttcattgctgcagatatcaaagggacagggtcctggacccagttgtacctaatcacagactatcatgaaaatggttccctttatgattatctgaagtccaccaccctagacgctaaatcaatgctgaagttagcctactcttctgtcagtggcttatgtcatttacacacagaaatctttagtactcaaggcaaaccagcaattgcccatcgagatctgaaaagtaaaaacattctggtgaagaaaaatggaacttgctgtattgctgacctgggcctggctgttaaatttattagtgatacaaatgaagttgacataccacctaacactcgagttggcaccaaacgctatatgcctccagaagtgttggacgagagcttgaacagaaatcacttccagtcttacatcatggctgacatgtatagttttggcctcatcctttgggaggttgctaggagatgtgtatcaggaggtatagtggaagaataccagcttccttatcatgacctagtgcccagtgacccctcttatgaggacatgagggagattgtgtgcatcaagaagttacgcccctcattcccaaaccggtggagcagtgatgagtgtctaaggcagatgggaaaactcatgacagaatgctgggctcacaatcctgcatcaaggctgacagccctgcgggttaagaaaacacttgccaaaatgtcagagtcccaggacattaaactctgataggagaggaaaagtaagcatctctgcagaaagccaacaggtactcttctgtttgtgggcagagcaaaagacatcaaataagcatccacagtacaagccttgaacatcgtcctgcttcccagtgggttcagacctcacctctcagggagcgacctgggcaaagacagagaagctcccagaaggagagattgatccatgtctgtttgtaggacggagaaaccgcttgggtaacttgttcaagatatgatgcatgttgctttctaagaaagccctgtattttgtgattgcctttttttttttttaagatgctttcattttgccaaaataaaacagataatgtggatggtttaagggttatagtattatagtttaaataataacaacaaaattcttcccaggaactctgctggaaggtaaattaaaatacttgtttttccattggtaaaatattgttgcactctgtgaaccaaaagacagtctaagttggaggacatagaacggaactcatcttaaacatactccccaccccgtcttggcctcctcagaccactttggccatccctgcatttggggccgctatggtaatgtgaatgcactgggtacaaacaccgcctgtctaggaccacatttggaattcctgcaggtggccttttgcagcttcaggcaatatggaacaaatgaaggtttatgtgactctaatagaagtaattgttgataggtgttcttcagatccacttctgtttctgattgagttaggcatctctttcatggtaaaacccttttcattaaacacaaagaaagcttttttttttttttttttttttttttttttttaatgtgcagaggattgacctgtgcatgcttttgatctctcattcaaaggatcaatattaaataaaattgtcatgagctgtgttgaagacagggtgctttcaaatagaggtaatttgctcttgtgttgtaagaggaacatgtcaacaaagataggaaatgagggtgatcgtgcagatggcttgtatcttatatatgcaaaggagccaatctcagaagcacaaagaaaaaagtgtgcataccttattttgtacagataaagatgatgtctttttgttattgtctgtctgttttgtatgtgtctgagataagggatagagaggaaacatccgtcaggctaatttaactacattttattttaaaaatagagaaacataacctctagatgggacagcagaggacagttagtagaggccacaaactgttatgggctgctgtgttttgttctaaaatcaatatggttggagcatgtatatcttaggtgatcatttcacatcttaggaatgcctactcattttattttattctagtgatgctcaattcactatttaatttattatattttctcttctgtggcacttatacaaaatatctcttcacctacttagttctacagggttttaactttggagcaacatgaataaaatcatcgagaaggccaatattgtttagcaacatgaatacaatacagtttaaagttgtacacatcctgctcaactttattcatatacatttcctttctgtggttttcttttgcttcttagaaattctgttagtggttagtaaagaatttgaaagtactttctccttgctgtttttttttttttttaagacattcctcccagaatactccagggggcagtgttttataacacattttccccactgggtgattgaaggatggaggatttttgaaaatttgacagctacatgaaacatgagaaaacattttcctcacttctgaagtcggtttgcagctggtaacttgttcatccagaaaacattctaaagcaatgagactttgtgagctgtgcttacagtttgggagaatcatgaagattctttctatattttgcatttacttcccagtgcttcatagctgcattttgtttgtaactaagacagaagaatttcgtaatccttgaaattgaaaaaaaaaaaattgtgtttttaaagagtgaaaacagttagaaaacaagtagaactgtaatcagaacgctgcttcaattgatattaaaaataacctcaataataatgtaaaggttcctttctcttgtgtcagttatattcttagggatagcctagaaggaatatatggttagaactaagtgtgactaatcatctgagccttgaagagaaacttcagtgcctctaaacagatcatctacaaaacaacaggtaaacatttatgccagttaagtgggtcatgtttttgtttcttgggtttttcctaaatttaagtgaggttgggcttaccttgtagataaaattatgttttctttttggtaaatacttgaacgtggataacgtcaaatcagaatattttgtgaggaggtgatgatttgaaattaagctagatttctagggaggtgttggttccaatgaaggatgggaagaaattaaaatagtcttcaaacttcttccttattatatttggttgctttggaaaagattggtcctatcctcaatctaatttattcactattaatattttaaaaacattcctgagatacttaaaaagacccacttagcgattatagttgctcaatgaaacaagaatttatttatgcatagatttttctctgtatcttaccaaaatccactttacttagataacactaaattgttcttaaagactactcatttcccaataatcctttatgatttcaaaatttctagtggctcagaagtgaattttattttatttgtctttcacttgaataaatgagaacccagaaattaataatgttgtttattgcttactgtcaggactatttcaaagactaagaagagtttcttctaacccctccctctcaaaggaatcctaaattattagttgttagataagttttgtatgctaagatattcaggtttatagtttatgtatgtgtgtatatatataaatatatatgtatatataaatattatgttcagtttggagtctggcacaactccattatgtggattagagagtaagatattatggatgataaagtactaaatgaaacataatatttatttataaaagtgtgtagattgttaaatcacaaaaagagtgctatgaccattatgtatgaggaaacaggcctttgacctcctggaaagcactgctcaaaagtcattagtgcccatttttgaattccccaaacagaaagcttcttagaaaacatgctgagattttatttacagggaattctttgacacatttcaattggtgtgtagtcaagtatagcaagtacttaataatgactgaatttcatgttcctacagtcatacatattcattagaagttttatgttgttggtctgatctgattcttctttgtttgtgggtggaacggcactgagagaagtatagttttttaaacttgaacatgttcagtagttacattgccttagaaaacccagacacatagcagtggaaatgaaagaaatggcatcagaagtgacttaatttagcaattgtgattcctcttgtaaaacaaaacaaaaaaacaatgccatattttttggagaaaagttggcaatataggggtttcgttgtctgtttcacaagaagactcatttgttcttttgggggaaccagtgccttacagattttgtatatactgtaattattcaggactagggaacaaacaattgtattgtatttgttacagattgtatatggctttgttttaacattcccctaaataaaatggcttcattctccccttggaaaaaaa

SEQ ID NO: 56 - DNA, 4016 bp, Homo sapiens
  source 1..4016: mol_type = genomic DNA
gccgtttctccgcgccgctgttggtgctgccgctgcctcctcctcctccgccgccgccgccgccgccgccgcctcctccggctcttcgctcggcccctctccgcctccatgtgccggatagcgggagcgctgcggaccctgctgccgctgctggcggccctgcttcaggcgtctgtagaggcttctggtgaaatcgcattatgcaagactggatttcctgaagatgtttacagtgcagtcttatcgaaggatgtgcatgaaggacagcctcttctcaatgtgaagtttagcaactgcaatggaaaaagaaaagtacaatatgagagcagtgagcctgcagattttaaggtggatgaagatggcatggtgtatgccgtgagaagctttccactctcttctgagcatgccaagttcctgatatatgcccaagacaaagagacccaggaaaagtggcaagtggcagtaaaattgagcctgaagccaaccttaactgaggagtcagtgaaggagtcagcagaagttgaagaaatagtgttcccaagacaattcagtaagcacagtggccacctacaaaggcagaagagagactgggtcatccctccaatcaacttgccagaaaactccaggggaccttttcctcaagagcttgtcaggatcaggtctgatagagataaaaacctttcactgcggtacagtgtaactgggccaggagctgaccagcctccaactggtatcttcattatcaaccccatctcgggtcagctgtcggtgacaaagcccctggatcgcgagcagatagcccggtttcatttgagggcacatgcagtagatattaatggaaatcaagtggagaaccccattgacattgtcatcaatgttattgacatgaatgacaacagacctgagttcttacaccaggtttggaatgggacagttcctgagggatcaaagcctggaacatatgtgatgaccgtaacagcaattgatgctgacgatcccaatgccctcaatgggatgttgaggtacagaatcgtgtctcaggctccaagcaccccttcacccaacatgtttacaatcaacaatgagactggtgacatcatcacagtggcagctggacttgatcgagaaaaagtgcaacagtatacgttaataattcaagctacagacatggaaggcaatcccacatatggcctttcaaacacagccacggccgtcatcacagtgacagatgtcaatgacaatcctccagagtttactgccatgacgttttatggtgaagttcctgagaacagggtagacatcatagtagctaatctaactgtgaccgataaggatcaaccccatacaccagcctggaacgcagtgtacagaatcagtggcggagatcctactggacggttcgccatccagaccgacccaaacagcaacgacgggttagtcaccgtggtcaaaccaatcgactttgaaacaaataggatgtttgtccttactgttgctgcagaaaatcaagtgccattagccaagggaattcagcacccccctcagtcaactgcaaccgtgtctgttacagttattgacgtaaatgaaaacccttattttgcccccaatcctaagatcattcgccaagaagaagggcttcatgccggtaccatgttgacaacattcactgctcaggacccagatcgatatatgcagcaaaatattagatacactaaattatctgatcctgccaattggctaaaaatagatcctgtgaatggacaaataactacaattgctgttttggaccgagaatcaccaaatgtgaaaaacaatatatataatgctactttccttgcttctgacaatggaattcctcctatgagtggaacaggaacgctgcagatctatttacttgatattaatgacaatgcccctcaagtgttacctcaagaggcagagacttgcgaaactccagaccccaattcaattaatattacagcacttgattatgacattgatccaaatgctggaccatttgcttttgatcttcctttatctccagtgactattaagagaaattggaccatcactcggcttaatggtgattttgctcagcttaatttaaagataaaatttcttgaagctggtatctatgaagttcccatcataatcacagattcgggtaatcctcccaaatcaaatatttccatcctgcgtgtgaaggtttgccagtgtgactccaacggggactgcacagatgtggacaggattgtgggtgcggggcttggcaccggtgccatcattgccatcctgctctgcatcatcatcctgcttatccttgtgctgatgtttgtggtatggatgaaacgccgggataaagaacgccaggccaaacaacttttaattgatccagaagatgatgtaagagataatattttaaaatatgatgaagaaggtggaggagaagaagaccaggactatgacttgagccagctgcagcagcctgacactgtggagcctgatgccatcaagcctgtgggaatccgacgaatggatgaaagacccatccacgccgagccccagtatccggtccgatctgcagccccacaccctggagacattggggacttcattaatgagggccttaaagcggctgacaatgaccccacagctccaccatatgactccctgttagtgtttgactatgaaggcagtggctccactgctgggtccttgagctcccttaattcctcaagtagtggtggtgagcaggactatgattacctgaacgactgggggccacggttcaagaaacttgctgacatgtatggtggaggtgatgactgaacttcagggtgaacttggtttttggacaagtacaaacaatttcaactgatattcccaaaaagcattcagaagctaggctttaactttgtagtctactagcacagtgcttgctggaggctttggcataggctgcaaaccaatttgggctcagagggaatatcagtgatccatactgtttggaaaaacactgagctcagttacacttgaattttacagtacagaagcactgggattttatgtgcctttttgtacctttttcagattggaattagttttctgtttaaggctttaatggtactgatttctgaaacgataagtaaaagacaaaatattttgtggtgggagcagtaagttaaaccatgatatgcttcaacacgcttttgttacattgcatttgcttttattaaaatacaaaattaaacaaacaaaaaaactcatggagcgattttattatcttgggggatgagaccatgagattggaaaatgtacattacttctagttttagactttagtttgtttttttttttttcactaaaatcttaaaacttactcagctggttgcaaataaagggagttttcatatcaccaatttgtagcaaaattgaattttttcataaactagaatgttagacacattttggtcttaatccatgtacacttttttatttctgtatttttccacttcactgtaaaaatagtatgtgtacataatgttttattggcatagtctatggagaagtgcagaaacttcagaacatgtgtatgtattatttggactatggattcaggttttttgcatgtttatatctttcgttatggataaagtatttacaaaacagtgacatttgattcaattgttgagctgtagttagaatactcaatttttaatttttttaatttttttattttttattttctttttggtttggggagggagaaaagttcttagcacaaatgttttacataatttgtaccaaaaaaaaaaaaaaaggaaaggaaagaaaggggtggcctgacactggtggcactactaagtgtgtgtttttttaaaaaaaaaatggaaaaaaaaaagcttttaaactggagagacttctgacaacagctttgcctctgtattgtgtaccagaatataaatgatacacctctgaccccagcgttctgaataaaatgctaattttggatctgg

SEQ ID NO: 57 - DNA, 9231 bp, Homo sapiens
  source 1..9231: mol_type = genomic DNA
gacttttctctgctctgcccgagagactgcagcggcggcggcgggagcgggcggacgcgcaggatgacgctgcggcttctggtggccgcgctctgcgccgggatcctggcagaggcgccccgagtgcgagcccagcacagggagagagtgacctgcacgcgcctttacgccgctgacattgtgttcttactggatggctcctcatccattggccgcagcaatttccgcgaggtccgcagctttctcgaagggctggtgctgcctttctctggagcagccagtgcacagggtgtgcgctttgccacagtgcagtacagcgatgacccacggacagagttcggcctggatgcacttggctctgggggtgatgtgatccgcgccatccgtgagcttagctacaaggggggcaacactcgcacaggggctgcaattctccatgtggctgaccatgtcttcctgccccagctggcccgacctggtgtccccaaggtctgcatcctgatcacagacgggaagtcccaggacctggtggacacagctgcccaaaggctgaaggggcagggggtcaagctatttgctgtggggatcaagaatgctgaccctgaggagctgaagcgagttgcctcacagcccaccagtgacttcttcttcttcgtcaatgacttcagcatcttgaggacactactgcccctcgtttcccggagagtgtgcacgactgctggtggcgtgcctgtgacccgacctccggatgactcgacctctgctccacgagacctggtgctgtctgagccaagcagccaatccttgagagtacagtggacagcggccagtggccctgtgactggctacaaggtccagtacactcctctgacggggctgggacagccactgccgagtgagcggcaggaggtgaacgtcccagctggtgagaccagtgtgcggctgcggggtctccggccactgaccgagtaccaagtgactgtgattgccctctacgccaacagcatcggggaggctgtgagcgggacagctcggaccactgccctagaagggccggaactgaccatccagaataccacagcccacagcctcctggtggcctggcggagtgtgccaggtgccactggctaccgtgtgacatggcgggtcctcagtggtgggcccacacagcagcaggagctgggccctgggcagggttcagtgttgctgcgtgacttggagcctggcacggactatgaggtgaccgtgagcaccctatttggccgcagtgtggggcccgccacttccctgatggctcgcactgacgcttctgttgagcagaccctgcgcccggtcatcctgggccccacatccatcctcctttcctggaacttggtgcctgaggcccgtggctaccggttggaatggcggcgtgagactggcttggagccaccgcagaaggtggtactgccctctgatgtgacccgctaccagttggatgggctgcagccgggcactgagtaccgcctcacactctacactctgctggagggccacgaggtggccacccctgcaaccgtggttcccactggaccagagctgcctgtgagccctgtaacagacctgcaagccaccgagctgcccgggcagcgggtgcgagtgtcctggagcccagtccctggtgccacccagtaccgcatcattgtgcgcagcacccagggggttgagcggaccctggtgcttcctgggagtcagacagcattcgacttggatgacgttcaggctgggcttagctacactgtgcgggtgtctgctcgagtgggtccccgtgagggcagtgccagtgtcctcactgtccgccgggagccggaaactccacttgctgttccagggctgcgggttgtggtgtcagatgcaacgcgagtgagggtggcctggggacccgtccctggagccagtggatttcggattagctggagcacaggcagtggtccggagtccagccagacactgcccccagactctactgccacagacatcacagggctgcagcctggaaccacctaccaggtggctgtgtcggtactgcgaggcagagaggagggccctgctgcagtcatcgtggctcgaacggacccactgggcccagtgaggacggtccatgtgactcaggccagcagctcatctgtcaccattacctggaccagggttcctggcgccacaggatacagggtttcctggcactcagcccacggcccagagaaatcccagttggtttctggggaggccacggtggctgagctggatggactggagccagatactgagtatacggtgcatgtgagggcccatgtggctggcgtggatgggccccctgcctctgtggttgtgaggactgcccctgagcctgtgggtcgtgtgtcgaggctgcagatcctcaatgcttccagcgacgttctacggatcacctgggtaggggtcactggagccacagcttacagactggcctggggccggagtgaaggcggccccatgaggcaccagatactcccaggaaacacagactctgcagagatccggggtctcgaaggtggagtcagctactcagtgcgagtgactgcacttgtcggggaccgcgagggcacacctgtctccattgttgtcactacgccgcctgaggctccgccagccctggggacgcttcacgtggtgcagcgcggggagcactcgctgaggctgcgctgggagccggtgcccagagcgcagggcttccttctgcactggcaacctgagggtggccaggaacagtcccgggtcctggggcccgagctcagcagctatcacctggacgggctggagccagcgacacagtaccgcgtgaggctgagtgtcctagggccagctggagaagggccctctgcagaggtgactgcgcgcactgagtcacctcgtgttccaagcattgaactacgtgtggtggacacctcgatcgactcggtgactttggcctggactccagtgtccagggcatccagctacatcctatcctggcggccactcagaggccctggccaggaagtgcctgggtccccgcagacacttccagggatctcaagctcccagcgggtgacagggctagagcctggcgtctcttacatcttctccctgacgcctgtcctggatggtgtgcggggtcctgaggcatctgtcacacagacgccagtgtgcccccgtggcctggcggatgtggtgttcctaccacatgccactcaagacaatgctcaccgtgcggaggctacgaggagggtcctggagcgtctggtgttggcacttgggcctcttgggccacaggcagttcaggttggcctgctgtcttacagtcatcggccctccccactgttcccactgaatggctcccatgaccttggcattatcttgcaaaggatccgtgacatgccctacatggacccaagtgggaacaacctgggcacagccgtggtcacagctcacagatacatgttggcaccagatgctcctgggcgccgccagcacgtaccaggggtgatggttctgctagtggatgaacccttgagaggtgacatattcagccccatccgtgaggcccaggcttctgggcttaatgtggtgatgttgggaatggctggagcggacccagagcagctgcgtcgcttggcgccgggtatggactctgtccagaccttcttcgccgtggatgatgggccaagcctggaccaggcagtcagtggtctggccacagccctgtgtcaggcatccttcactactcagccccggccagagccctgcccagtgtattgtccaaagggccagaagggggaacctggagagatgggcctgagaggacaagttgggcctcctggcgaccctggcctcccgggcaggaccggtgctcccggcccccaggggccccctggaagtgccactgccaagggcgagaggggcttccctggagcagatgggcgtccaggcagccctggccgcgccgggaatcctgggacccctggagcccctggcctaaagggctctccagggttgcctggccctcgtggggacccgggagagcgaggacctcgaggcccaaagggggagccgggggctcccggacaagtcatcggaggtgaaggacctgggcttcctgggcggaaaggggaccctggaccatcgggcccccctggacctcgtggaccactgggggacccaggaccccgtggccccccagggcttcctggaacagccatgaagggtgacaaaggcgatcgtggggagcggggtccccctggaccaggtgaaggtggcattgctcctggggagcctgggctgccgggtcttcccggaagccctggaccccaaggccccgttggcccccctggaaagaaaggagaaaaaggtgactctgaggatggagctccaggcctcccaggacaacctgggtctccgggtgagcagggcccacggggacctcctggagctattggccccaaaggtgaccggggctttccagggcccctgggtgaggctggagagaagggcgaacgtggacccccaggcccagcgggatcccgggggctgccaggggttgctggacgtcctggagccaagggtcctgaagggccaccaggacccactggccgccaaggagagaagggggagcctggtcgccctggggaccctgcagtggtgggacctgctgttgctggacccaaaggagaaaagggagatgtggggcccgctgggcccagaggagctaccggagtccaaggggaacggggcccacccggcttggttcttcctggagaccctggccccaagggagaccctggagaccggggtcccattggccttactggcagagcaggacccccaggtgactcagggcctcctggagagaagggagaccctgggcggcctggccccccaggacctgttggcccccgaggacgagatggtgaagttggagagaaaggtgacgagggtcctccgggtgacccgggtttgcctggaaaagcaggcgagcgtggccttcggggggcacctggagttcgggggcctgtgggtgaaaagggagaccagggagatcctggagaggatggacgaaatggcagccctggatcatctggacccaagggtgaccgtggggagccgggtcccccaggacccccgggacggctggtagacacaggacctggagccagagagaagggagagcctggggaccgcggacaagagggtcctcgagggcccaagggtgatcctggcctccctggagcccctggggaaaggggcattgaagggtttcggggacccccaggcccacagggggacccaggtgtccgaggcccagcaggagaaaagggtgaccggggtccccctgggctggatggccggagcggactggatgggaaaccaggagccgctgggccctctgggccgaatggtgctgcaggcaaagctggggacccagggagagacgggcttccaggcctccgtggagaacagggcctccctggcccctctggtccccctggattaccgggaaagccaggcgaggatggcaaacctggcctgaatggaaaaaacggagaacctggggaccctggagaagacgggaggaagggagagaaaggagattcaggcgcctctgggagagaaggtcgtgatggccccaagggtgagcgtggagctcctggtatccttggaccccaggggcctccaggcctcccagggccagtgggccctcctggccagggttttcctggtgtcccaggaggcacgggccccaagggtgaccgtggggagactggatccaaaggggagcagggcctccctggagagcgtggcctgcgaggagagcctggaagtgtgccgaatgtggatcggttgctggaaactgctggcatcaaggcatctgccctgcgggagatcgtggagacctgggatgagagctctggtagcttcctgcctgtgcccgaacggcgtcgaggccccaagggggactcaggcgaacagggccccccaggcaaggagggccccatcggctttcctggagaacgcgggctgaagggcgaccgtggagaccctggccctcaggggccacctggtctggcccttggggagaggggcccccccgggccttccggccttgccggggagcctggaaagcctggtattcccgggctcccaggcagggctgggggtgtgggagaggcaggaaggccaggagagaggggagaacggggagagaaaggagaacgtggagaacagggcagagatggccctcctggactccctggaacccctgggccccccggaccccctggccccaaggtgtctgtggatgagccaggtcctggactctctggagaacagggaccccctggactcaagggtgctaagggggagccgggcagcaatggtgaccaaggtcccaaaggagacaggggtgtgccaggcatcaaaggagaccggggagagcctggaccgaggggtcaggacggcaacccgggtctaccaggagagcgtggtatggctgggcctgaagggaagccgggtctgcagggtccaagaggcccccctggcccagtgggtggtcatggagaccctggaccacctggtgccccgggtcttgctggccctgcaggaccccaaggaccttctggcctgaagggggagcctggagagacaggacctccaggacggggcctgactggacctactggagctgtgggacttcctggaccccccggcccttcaggccttgtgggtccacaggggtctccaggtttgcctggacaagtgggggagacagggaagccgggagccccaggtcgagatggtgccagtggaaaagatggagacagagggagccctggtgtgccagggtcaccaggtctgcctggccctgtcggacctaaaggagaacctggccccacgggggcccctggacaggctgtggtcgggctccctggagcaaagggagagaagggagcccctggaggccttgctggagacctggtgggtgagccgggagccaaaggtgaccgaggactgccagggccgcgaggcgagaagggtgaagctggccgtgcaggggagcccggagaccctggggaagatggtcagaaaggggctccaggacccaaaggtttcaagggtgacccaggagtcggggtcccgggctcccctgggcctcctggccctccaggtgtgaagggagatctgggcctccctggcctgcccggtgctcctggtgttgttgggttcccgggtcagacaggccctcgaggagagatgggtcagccaggccctagtggagagcggggtctggcaggccccccagggagagaaggaatcccaggacccctggggccacctggaccaccggggtcagtgggaccacctggggcctctggactcaaaggagacaagggagaccctggagtagggctgcctgggccccgaggcgagcgtggggagccaggcatccggggtgaagatggccgccccggccaggagggaccccgaggactcacggggccccctggcagcaggggagagcgtggggagaagggtgatgttgggagtgcaggactaaagggtgacaagggagactcagctgtgatcctggggcctccaggcccacggggtgccaagggggacatgggtgaacgagggcctcggggcttggatggtgacaaaggacctcggggagacaatggggaccctggtgacaagggcagcaagggagagcctggtgacaagggctcagccgggttgccaggactgcgtggactcctgggaccccagggtcaacctggtgcagcagggatccctggtgacccgggatccccaggaaaggatggagtgcctggtatccgaggagaaaaaggagatgttggcttcatgggtccccggggcctcaagggtgaacggggagtgaagggagcctgtggccttgatggagagaagggagacaagggagaagctggtcccccaggccgccccgggctggcaggacacaaaggagagatgggggagcctggtgtgccgggccagtcgggggcccctggcaaggagggcctgatcggtcccaagggtgaccgaggctttgacgggcagccaggccccaagggtgaccagggcgagaaaggggagcggggaaccccaggaattgggggcttcccaggccccagtggaaatgatggctctgctggtcccccagggccacctggcagtgttggtcccagaggccccgaaggacttcagggccagaagggtgagcgaggtccccccggagagagagtggtgggggctcctggggtccctggagctcctggcgagagaggggagcaggggcggccagggcctgccggtcctcgaggcgagaagggagaagctgcactgacggaggatgacatccggggctttgtgcgccaagagatgagtcagcactgtgcctgccagggccagttcatcgcatctggatcacgacccctccctagttatgctgcagacactgccggctcccagctccatgctgtgcctgtgctccgcgtctctcatgcagaggaggaagagcgggtaccccctgaggatgatgagtactctgaatactccgagtattctgtggaggagtaccaggaccctgaagctccttgggatagtgatgacccctgttccctgccactggatgagggctcctgcactgcctacaccctgcgctggtaccatcgggctgtgacaggcagcacagaggcctgtcacccttttgtctatggtggctgtggagggaatgccaaccgttttgggacccgtgaggcctgcgagcgccgctgcccaccccgggtggtccagagccaggggacaggtactgcccaggactgaggcccagataatgagctgagattcagcatcccctggaggagtcggggtctcagcagaaccccactgtccctccccttggtgctagaggcttgtgtgcacgtgagcgtgcgtgtgcacgtccgttatttcagtgacttggtcccgtgggtctagccttcccccctgtggacaaacccccattgtggctcctgccaccctggcagatgactcactgtgggggggtggctgtgggcagtgagcggatgtgactggcgtctgacccgccccttgacccaagcctgtgatgacatggtgctgattctggggggcattaaagctgctgttttaaaaggc

SEQ ID NO: 58 - DNA, 4624 bp, Homo sapiens
  source 1..4624: mol_type = genomic DNA
gagagcgcggtggagagccgagcgggcgggcggcgggtgcggagcgggcgagggagcgcgcgcggccgccacaaagctcgggcgccgcggggctgcatgcggcgtacctggcccggcgcggcgactgctctccgggctggcgggggccggccgcgagccccgggggccccgaggccgcagcttgcctgcgcgctctgagccttcgcaactcgcgagcaaagtttggtggaggcaacgccaagcctgagtcctttcttcctctcgttccccaaatccgagggcagcccgcgggcgtcatgcccgcgctcctccgcagcctggggtacgcgtgaagcccgggaggcttggcgccggcgaagacccaaggaccactcttctgcgtttggagttgctccccgcaaccccgggctcgtcgctttctccatcccgacccacgcggggcgcggggacaacacaggtcgcggaggagcgttgccattcaagtgactgcagcagcagcggcagcgcctcggttcctgagcccaccgcaggctgaaggcattgcgcgtagtccatgcccgtagaggaagtgtgcagatgggattaacgtccacatggagatatggaagaggaccggggattggtaccgtaaccatggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatgaatgactgtgtctgcctgtccccaaacaggacagcactgggaacctagctacactgagcagggagaccatgcctcccagagcttgttgtctccacttgtatatatggatcagaggagtaaataattggaaaagtaatcagcatatgtgtaaagatttatacagttgaaaacttgtaatcttccccaggaggagaagaaggtttctggagcagtggactgccacaagccaccatgtaacccctctcacctgccgtgcgtactggctgtggaccagtaggactcaaggtggacgtgcgttctgccttccttgttaattttgtaataattggagaagatttatgtcagcacacacttacagagcacaaatgcagtatataggtgctggatgtatgtaaatatattcaaattatgtataaatatatattatatatttacaaggagttattttttgtattgattttaaatggatgtcccaatgcacctagaaaattggtctctctttttttaatagctatttgctaaatgctgttcttacacataatttcttaattttcaccgagcagaggtggaaaaatacttttgctttcagggaaaatggtataacgttaatttattaataaattggtaatatacaaaacaattaatcatttatagttttttttgtaatttaagtggcatttctatgcaggcagcacagcagactagttaatctattgcttggacttaactagttatcagatcctttgaaaagagaatatttacaatatatgactaatttggggaaaatgaagttttgatttatttgtgtttaaatgctgctgtcagacgattgttcttagacctcctaaatgccccatattaaaagaactcattcataggaaggtgtttcattttggtgtgcaaccctgtcattacgtcaacgcaacgtctaactggacttcccaagataaatggtaccagcgtcctcttaaaagatgccttaatccattccttgaggacagaccttagttgaaatgatagcagaatgtgcttctctctggcagctggccttctgcttctgagttgcacattaatcagattagcctgtattctcttcagtgaattttgataatggcttccagactctttggcgttggagacgcctgttaggatcttcaagtcccatcatagaaaattgaaacacagagttgttctgctgatagttttggggatacgtccatctttttaagggattgctttcatctaattctggcaggacctcaccaaaagatccagcctcatacctacatcagacaaaatatcgccgttgttccttctgtactaaagtattgtgttttgctttggaaacacccactcactttgcaatagccgtgcaagatgaatgcagattacactgatcttatgtgttacaaaattggagaaagtatttaataaaacctgttaatttttatactgacaataaaaatgtttctacagatattaatgttaacaagacaaaataaatgtcacgcaacttatttttttaa

SEQ ID NO: 59 - DNA, 2256 bp, Homo sapiens
  source 1..2256: mol_type = genomic DNA
ggggcgggggaggatcacgagttcgggagttcgagaccagcctggctaacatagttgaaaccccgtctccactaaaaatacaaaaattagccaggtgtggtggcgcgcgcctgtagtcccagctactctggaggctaaggaaggagaatcgcttgaatccgggaggcggaagttgcagtgagccgagatcgcaccactgtacacctgggtgacagagtgagactctgtctcaaaaaaaaaaaaaaagaaaaaaagccggccgggtatagtggctcacgcctgtaatcctaacactttgggaggctgaggtcgggagttcgagaccagcctggccaacatggagaaaccccgtctctactaaaaatacaaaaaaaaaaggagccggggatggtggcgcatgcctgtaatcccagctacttgggaggctgaggcaggagaatcgcttgaacctgggaggcggaggttgcagtgagccgagatcgcgccattgcacttcagcctgggcaacaagaacgaaactccgcctcaaaaaaaaaaaaaaaaaaaaaaaaaagccgtgggtcaggagccagagccctctgctctagtcttagctgtgccaatgtgactccctgtgtgatttgggggtaaattactgcccctcactgggtctctgtctacccacctgcaaaatgagaggatctttggatgagtctccttaattcatttcctggacaaacttcctccaaggctcccccagatttaccagtgacgcccttccctaagtatcaacttagccgtactttgaactctggcccctcaggtttcaaggggacggccttgtctgggatcagctctgtctggggaaagaagctgcaccagcctctggatagcaggctgagtcacttgtttcttgtgccttgagtcagttctctcatcagctctcctcctaggccagtgttattacctccagtaaagatggaaaagttggggttcagagagggctagagacagacagcctgctgtgtcctgtcttttctcctgtgctgtacaggttcaccaatttttaaaatccaaatatatctcatggtacagtggaagaactggccagagagtctggaagtttgggttctggtcctggctgtgccactgactcactgtgaccttgggatcttgtgctgtgaagacatttcccaagtgcttcatgttagccagcaaatctgacccacaaggcctggaaagaggtgattgttaggttgcgcagaggtggtcttatccagctcagcttcccctgggacccaccgtgggacctgaggcagaactggggtggacttggcctcctccatggcacaccggctgcagatacgactgctgacgtgggatgtgaaggacacgctgctcaggctccgccaccccttaggggaggcctatgccaccaaggcccgggcccatgggctggaggtggagccctcagccctggaacaaggcttcaggcaggcatacagggctcagagccacagcttccccaactacggcctgagccacggcctaacctcccgccagtggtggctggatgtggtcctgcagaccttccacctggcgggtgtccaggatgctcaggctgtagcccccatcgctgaacagctttataaagacttcagccacccctgcacctggcaggtgttggatggggctgaggacaccctgagggagtgccgcacacggggtctgagactggcagtgatctccaactttgaccgacggctagagggcatcctggggggccttggcctgcgtgaacacttcgactttgtgctgacctccgaggctgctggctggcccaagccggacccccgcattttccaggaggccttgcggcttgctcatatggaaccagtagtggcagcccatgttggggataattacctctgcgattaccaggggcctcgggctgtgggcatgcacagcttcctggtggttggcccacaggcactggaccccgtggtcagggattctgtacctaaagaacacatcctcccctctctggcccatctcctgcctgcccttgactgcctagagggctcaactccagggctttgaggccagtgagggaagtggctgggccctaggccatggagaaaaccttaaacaaaccctggagacagggagccccttctttctccacagctctggacctttccccctctccctgcggcctttgtcacctactgtgataataaagcagtgagtgctgagctctcacccttcccccactaa

SEQ ID NO: 60 - DNA, 2967 bp, Homo sapiens
  source 1..2967: mol_type = genomic DNA
gagctcctctgctactcagagttgcaacctcagcctcgctatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggccccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtcctaatcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactatgcagctccagtttcctgcagtgatcagggtcctgcaagcagtggggaagggggccaaggtattggaggactccctcccagctttggaagcctcatccgcgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttccagagacggggtctcgcaacattgcccagacttcctttgtgttagttaataaagctttctcaactgcc

SEQ ID NO: 61 - DNA, 9324 bp, Homo sapiens
  source 1..9324: mol_type = genomic DNA
gggcatgctcggcctcggcggctgcaggcggggcgggtcgcgctggtccccggccggccggagcggcggcggtggcggcgcgaggcggcatgagggagccgctcgtgctgggtcctgccaccaccatggttccagtggttgagctttgctgaagcccaggcatcatgtacagtgtagaagacctcctgatctctcatggatacaagctgtcaagagaccccccagcatcacgcgaggataaccccaaggggcgccaggcagcgaggactgggacacgagcaggccagggcctgcagaacgggcatgaggatggccctgcggccctcgcacatcgtaagacgtccgcggggaaaggacatgtgagtgactccgaaagccgccgcagcacaccgagaggccacggggagccccagagcacttctgcttccagaacctcggaggcggggttttgtaatcaacccccctcagcatggtcctctcatcccccgactggtaacgaccaagcctaccggagaagaggacggcaagaagccaggagccagaagccgagggagcacgaaaacctggaggccagaggaatggcccaagcccacagcctgcctgtccacgtgagggagggtccatgggaagttggaggaaggtcagagcatgtgatgaagaagccagtttgggaagaagaattgcgaatgtcaggtcctgccaagtggcagaacgtcagcctggaaagctggaaccagccaaggaaattagggaggcagatgtctgatggagatggggagagactgtttcaagacctgtacccattcattcaaggagaacatgtgttgaattctcaaaacaaagggaagtctcgctcactgcctagagttctttcccccgagagcctgagttgcacggaaattcccattccattaaatgaaagacattcacctaaaatgccaccgtatcctcccacttgcgcaccaaatttggactccacgaggaattctgagaagagtggctgctcagccccatttccccggcctaagtttgggaggcccctcaagcccccatcttacagctcgcaccagcagtctaggggaggagcggacagcagtgactctcaggacagccagcagatggacgcctatgtccccaggcatgagctctgcctgtcagaccctggattggaacctccagtgtacgtgcctccgccctcatacagatcgcccccgcagaacatcccaaacccctacttggaagacacggtgcccataaatgtgtgtggcggtcacagtcaacagcagtctccgaccgagaaggctggggccagcggtcagcctccttcaggcccccctggaactgggaatgagtatggtgtgagcccccgcttgcctcaggggctccccgcacatccccgacctgtcactgcctatgacggcttcgttcagtacattccctttgatgatccacggttacgacattttaaactagctcagccccagggtttctgtgaagacataaagcttgacgataaatcatataactccagtcctgtcactgctcaagagccggctcatggaggaatgcagcctgatggtgccatttggaatccacagagcttaatacccccgtcgggggatgagagaggcctggtcttggccgattccagcccccggtggctgtggggccagccccccggggatggggaaaacagtggcctccccaaccagagagaccgctgtgtggcaaggggacagtggcctgatgtgagaggcagccagcacgggcacactggaagacaagtttcctccccttactcacagggcgagagcacctgcgaaactcaaaccaagctcaaaaagttccaaactgggactcggaccaagaaaagttcaaagaaaaaaatgaacgagactatattttgcttggtttctatcccagtgaaatcagaatcacatctgccagatagagatatggacaacaatgacttaaagcccagtgctgatcaaaagaatgggtctgataagagcccggctctgcaagaacagagtctgctgagcatgtcttccaccgacctggagctgcaggccctcacaggaagcatgggtgggagaacggagttccaaaaacaagatctaggggaaccagaagaagacagacaaacaaatgacctcagtttcatccaccttacaaagcacagagaactcaagcattctggctcttggccagggcaccggtacagagatcagcaaacacaaaccagtttctccgaggagccccaaagttcgcagctgctccctggtgcaaagctgggagggccgagtcgtgcagcattgagtccaaaatgttcagaccctgctgcctccgaagctcagacgcacacagcattccctaccggtgatcacaaacagaggccaagtgcccgtaacctgaaaggtcacaggtccctcagcccatccagcaacagtgcgttctcaaggacttccttgtccgtggaccaggcaccgacgccaaaagcaggccgaagtcagccctgcgtggatgtccacgggcttggagcccaccctgggcctaagcgggaggtggtgaagggggagcccacgggcccttgcaacagtaaacaactctttgggcagtttctcctgaaaccggtcagccgtcgtccctgggatttgatcagtcagttagaaagttttaacaaggagctccaggaagaggaagaaagcagcagtagcagcagcagcagcagcagcagcagtgaggagagtgaggcggagccgcagcaggagaaccgtgctcactgcagacaggaggatgtgggcttccgcggaaacagcccggaaatgagggttgagccacagccgaggatgtgggtgccggagagccctgtgtgtaggtcgggaagaggtgagagtaagtctgagagctggagtgaggagctgcagcctggccacccacgtgcctggcctccatccccgggccgctttcgcgtggaagaaggtggcggtgcacctttctgctcagcagatggaagcacgagtgcagagaagagacacctggaggttagcaacggaatggacgagctggcaggtagcccatttcctgtgacgagaatgtcttcaagatcaagtgacgcaaaaccactgcccgcgtcctatccagctgaacctagggagccccaggaaagtccgaaaatcaccagtgctttcagctctgtgaaaccaagtgaagcggtccctcggaagtttgacagtggtggagagaggggggcagggctcccactgtccctgtctaacaagaaccgagggctctcagctccagacttacggtctgtggggctcacccctgggcaagaacagggtgccagtgagctagaggggtctttgggtgaagcaagcacaatagaaatccccccaggtgagtccttgcaagccagggctgcaaggatcctgggcattgaggtggcggtggagtccctcctgccgggcatccggagagcgggacagaaccagcctgctgagcccgatgcaagtgcctgcaccccagagtccccccaggaagagttgctatctcgcccagcaccggcagatgtccccagggtgtccactgatgccttttatggcaggaggaagtgcggctggaccaagagccctctctttgtaggggacagggacagtgccaggcgggctcctcaggcttttgagcactcagatgtggacggggttgtcaccagcacagaccctgtccctgagcctgagcccagccccttggagtccaagttcttcgaacaaaaggatgtggaaacaaaaccacccttcaggtccactttattccattttgtagaaagaaccccaagtgtggcaggctctgaaaagagacttagaagcccttccaaagtgattgaaagtttacaagagaaactggcctccccgcctaggagagcagaccctgaccgcctgatgagaatgaaagaggtgagctcagtgtcacggatgagagtcctgagcttcaggaatgccgactcccaggaggacgccgaggaattgaaggccaccacaaggggccaggccgggctcccgggaggccttgtgtctcctggcagtggggaccgtgcccagagattgggccactcactctctgtgtccaaggacagcatctccagggaagagaaggagcatccggcagcacaaaaggagaagagcatggatcaagacttctggtgcccagattcctatgaccctagcagagtggagagggtgtgatgaaatgccggtggagcttgcattccgttgcgaattgagtattctcaagtagctggtagatttagtctatcagccccacttccccatgtagaagctggaagctgctgagtttttagcatcctgataacattctttaccactgccactaggaaacttgccatcctctcagaattgctttgtggcgataaagcaatcgtccgttgaaaccatctgtttttatagcatgaagatttaatgcctataattaggaaggagttttcacagaaaccctccctcatgacagatttgactaagctggtaaatagtttggtgttgctctcattctgggtgaagatttcaatgtggaagatctagcaaatgctttggaaaggcctgtgaatggtgtgctcccctctgtgtctccatggcctgcagtccatgctcttgtgaaggagtttgggacactggctgttgcccggtgtttcagcactttcagtttaggggaaatgtgaatcaataggtatgatccatttctaatcaatttggccttctgctgctggaagtaactttacttataagtaacttgccatcagagaataaaagcaatatcttttaagttcctgaaaaattaaaacttgtactgtaatatactaagagtattatttatctggcagtgaaatattttgatcatttgcaaacacgccattgtctgctttgcactcagtattatctatttttattttctgtatgggtatttagaaattctttatatgaaaaataattgctggtttaaaatagaccattttaacaaagtgactatatttctttttacgaaaatgctatttttctatgatgtaaacaattgttaggcggcagatttttaaggaagtattattttatttaagagtctgtatccctttgtgtgagaggaacccaaagagggttcacttttgctttaaagctctgtcccttacccactgacaactctgctacttttattttatcttagaaaatgtcaatatagacatatcacagggttaataaaagaaacttttcacaaagatttttgtacaagtgtagctatcaggttatatttgaaaataaccctctgaaaaccagccaacattgtcatgacaatttttgctgccgtatcaactagaattaccaaaaaacatagttttatctttgtatgtgtgtggggatctaccctgagaccatgcttttcttcccaccctgccaccccacccctttaaagaaatacgtatttttatctagaatgatcttgaattagtctctgcatatttttagaaagtgcttggggacattatatatatgccattacaaaagcattatgaggcctatgtattctactgtgtttaatactgcattaacctgcacaaagtttatgcattttttcatgcatttttttccttctttctaagtaaggtattcctgacaactaacattccctaaatcatttcccctccctccctccctccctcccttcctttcttccttccttctgtcctccctcccttgctacctcttgccctccttccttcaccctgtcctccttttcccttttattttttccccttctctttcattttattttcttttagagaaaccaccttatctatttttagacatttcgaattcctaaaatgtgaaccgttgggtgatctgtcctttggccctaaaacggctggccgggctgtcccggcctgttctcatctgtaccgtatgattttatttcataataactttatgtatttggactgtaacaaacaaaacagaacctaagtcgccccgaaaaatctttccaagccttttcaaaacacttatctgcagtccagaattcagctcttatgaaaagtgagagtgcactccagttttcacagcagcaataagtgcgggaaacaaaaagatcctcttcccaggaaaagttgagtggggaagagttcagacactgttgtagctggtgaatcattgaagtcacctggtagtaccaaataagggctgaggcgcctggaggtctttgtggggtcttccctctttaaatgcaaacgtaagaaggtatatgtgtatatacatttatatacgtgcacatgtatactcgtacatgtatgcacacatacccatacatgtatatgaactccgttctgatttcctctgaaattttgttgataccatctaaatacctctttgaatcaaaataacttaattttagctcattcctcaagagaaagagaaggatccccaaggaagaaaaactgttgtaccttctgtgattctcaaaccaaaatgtgatgcgcttctgttttgttatcttaaattgttgtatcatatctttctgaaaccattctgaattaagttgaaattttcaacatttatactttaactgttctaaatttaagcctacatgtaaatcttttttggggggaaggtaacttgaaaagtttaacttgaaaatttgacctttaaactttcatattctaaggtaaataatagcctataaccaacaacagctttcccagaggtcttgaatttaacactaattgtttagttattgtttctatttctcatacatacataacttcgttttttgtggctggaagaaaaagtttctatattttgtatttgtctttgttcttctcatgcacacaatttactgttggtgtttaataatttggagctcataaaattaaaagttgtaattttgaccatttgggctaaaaattatatgagggattacatatgcaatggattagtatttcttgatttgcaaaagaagggaaaatgatggtaatttagctcaaattgcctttctttgaagaaaatttctgaatgactgaaaaccaaatatctatattatcactttcaacctcatgtttctgcaaagtacagaaaaactcctcactgacagtgcacccagcccctgactgggttttcaggattatgtccatgtgcataactgcttggatttcttcttttaaagtccctaaaacatgaatctgttctttgagatcctggtttccttagcagagttgtaggttgcgggtttcattcacgtggaaattctaagtagaacttttgctgccccattttactgttggtgaagggccgttgttaatggaagtttacgttttaaaactcagatgactcagaaaccagactgtgatcattttttgcagcagcaaaatgacttattctgacagcatctcggtttggggcttctgaagaaaaaggatgaagctgatctgttgaaacaactactttcccaactcggaggcaaaaggggctgatagaagataacattcacctaccatttcatcttgtgacccccagagatcacgtttgcgtgttctggctgagttttttattattgtttgttttaaatgattaaataatacacaattaaatgtcaggatagcatctcttgcaaagtgaccagtgcagcagaattgtctttctatccatatttgagcttagagaaacatttcccatgtcctaagttcttagaagcaattactttagcgttggggagcattgttctaccagacccatttatgcaggggagatgaagcttaaaagacgggatgtggggtgggagtgtgtttcttaagccgaagctgttgcaggctgggggatttttgcattttctttttgttttgtttttgactgcagattctgtacatctgtctgtgggaggagagttgctactcaggacaggatttaagagacacattccagtgacctttaagaatctgcatggcgggaggtccttctccaggagtgtgggttggtccactctgggacccaccacactaagaagggggagatgataaaataacattaaaggaagaatggcctccagcctgcaggttttgttggaaagaaataaaaagggagtcattaagaccataaattcagattgaggcctcttgaaaaggttgatgtgggctagcaacctgcctgtcgaaacagtccttggttcatcagcagtgtgggaaggcagccaaggctcctgcagattcctggcatcgaccttggaaagcctctgcgatacttgtgtgtacgaatacagcagaggacagggaggtccttgttctgtggtctctgtttagtgactgaaacttaaacccaaaggcaagcccagattgtctggccgttcatccccatgctttgataggggttaggaggaaccctttccgtatgaaagacaggccctactagggcttacaaccaagccaaaggaccatctcttctttcttccacctcccttcacccctgccccgcagcagagccgagatgtgagacattcattgtcacggagcaaggagacaaaggcaagttcaagttgagaagcatatggcagcaaacagaaatgaaaaccatatgtcccagcaaggggaaaagcagtcatttccagattataaaaatcaatgaagtactctccacctaggtcagctgaaattcgagccctcacagtcaggcctgtcagagaagttaagcagaaacatctcggggggacttctaaaatttagtgaagacaaggccttgcaactccaaagaaactttttttcccccccttgaaacagggtcttgctctgttgcccagcctggagtgcagtggtgcagtcacggctcactgcagcctcaagctcctgggctcaagcactatccccacctcaacctccttagtagctgggactacaggtgcacaccaccatgcccagctaaccacagaaactttcatctcttcattttttctttgggcaccattaatacctaagacaggtagaaagggtcccagaaagacaccattggtaatggccgattgccggctgcagtcatcgcccccagatcaggctggtacaggatgccttaaggtgatgagaggtgagggtgcatgaagaataatgagcacagggaagagagaagcaggacaaagtagcagataaaatgccggcaaagcacagatgaatgtcttcaagaagctcttgtatttctctgcacagtgtaaatatccttgctatttcaggatggcggctggcctgctcagtaacatacatgttccaaataaagattttgcatgaaa

SEQ ID NO: 62 - DNA, 3499 bp, Homo sapiens
  source 1..3499: mol_type = genomic DNA
gcagtccgactcatcccggccccgggatggcgtccccacgggaattgacacagaaccccctgaagaagatctggatgccatacagcaatgggcggcccgctctgcacgcttgccagcgcggtgtgtgcatgaccaactgcccaactctcattgtcatggtgggcctgcccgccaggggcaagacctacatctccaagaagctgactcgatacctgaactggattggtgtgcccactcgggagttcaatgttggccagtatcgccgggacgtggtcaagacctacaaatcttttgaattttttctccccgacaatgaagagggcctgaaaatcaggaagcagtgtgccctggcagccctccgtgacgtccggcggttccttagtgaggaggggggacatgtggcggtttttgatgccacaaacaccacccgagaacggagagcgaccatctttaattttggagaacagaatggctacaagaccttttttgtcgagtccatctgtgtggatcctgaggtcatagctgccaacatcgtgcaagtgaaactgggcagccctgactatgtcaaccgcgacagtgatgaggctacggaggacttcatgaggcgcattgagtgctatgagaactcctacgagtcgctagatgaggacctggatagggacctgtcctatatcaagatcatggatgtgggccagagctacgtggtgaaccgtgtggctgaccacatccagagccgcatcgtatattacctcatgaacatccacgtgaccccccgctccatctacctctgccggcacggggagagcgagctcaacctcaagggccggattggcggggacccaggactgtcccctcggggcagggagtttgccaagagtctagcccagttcatcagtgaccaaaatatcaaggatctgaaggtctggacaagccagatgaagaggacaatccagacggctgaggcactgggtgtgccctatgaacagtggaaggtcctcaacgagatcgatgcgggcgtctgtgaggaaatgacctacgaggaaattcaggataattatccactggagttcgccctgcgggaccaggacaagtaccggtaccggtaccctaaaggggagtcctacgaggacctggtccagagactggagcctgtcatcatggagctggagaggcaagagaatgtgctggtcatctgccaccaggctgtgatgcgctgcctgctggcctacttcctcgacaaggcagcagaacagctgccctacctcaagtgtccgctgcacacagtcctgaagctgactcctgtggcatatggttgtaaagtggagtccatattcctgaacgtggctgctgtgaacacgcaccgggacaggcctcagaacgtggacatctcaagacctccagaggaagcccttgtcacggtgcctgctcaccagtgaccatgttcatccactgtgaccactaggcaggcactgctctctgcagagggggtcattccaggccctccagtgtgtgtgatagtcaccatgccatgcagggatattcttgaagccacacatggctggcggaacccagagcccccaccccagcccacctggctctttgttgacagtcggcgacaaggttgtgcgtggctcctgacctgctgctaagagtcacttgaccagactgcatctgcatgggctgcgcggaggttgcccagccccagtttcttccggcgcagctcttaggtgttcactctcgccagctcagttggctttgtgaagtgtgaaaccctacaatgtgaaaggaaagtgcttgctgtgatgttcctactgtggcccagctgcccagcatggacctggtgactctccacagggcctctaccatcctctctgtggccacttcctgagccagaggccaggtcttcatggggccctgagcttctgctgcctctggtgagagggagagcccttcccatccttacccaccaggaactagagccccaaccacagcagatgcttcctgggcagccactggccaggccgttgtatccatgtcacccttagttgtgggcattcatgaaagcaatgcgcttgcttcagcacattgggatgcacagaaacgtgagggcaggggggcagttgtggtcgcccctccccagttgtgcatgtacccggctacttaggtatgcttaggggagctggtggccaggggtgccccttcaggcctgctctcacagtgaaaccacttcaagacagggcaggaagttaccttcctggtgcacacaccgaagtgctcgagggagaggctgccgccccctctccccttccctggctgtggacactggcagccaatggggaggcggcctctcccctgttgctgcccgtgtccacacaggaatgtacactggtggcagaaacatgcatttgccataaataattcagaaacacttgtatggtcctgtaaaacttctctggtgtttgttgacttctgcctgtgggtctgaggaagggagaaggcagaacccataggcagtccctgggcaagggaggattttcccggccgcagcacagccttccattcatgtgcacgtgagtctggtgaaaatggccttgtgaagtccagtgtctgtcatgcccctgagttcttttggtcccagagaggcagcctgggcagtctgtttacagctggcaaacagactggctggcaagttgtggggctgggtcagaaagtgtctggacttgtaatcgggtgccctgctgtccacaggtggactctggggaagccaggcccctggatcccaagtcctttgtgtccggagcctttcctggcctgtgcctggagggtagcacatctttccagcctcccagggctttgcagcctgcagatgccacctgtccctgagagaagttagcgtccctggccctggccggtggtctctcatccttgtgtgctgctctgctaagagatgtccaaggcggagccggggcaagatccttccagactcatctgtcagagccccaagccctttagacccagagcccaaggaccatgcctttgggacattaggactgcagcctttgcttctgtgtattttggagttttggtgacttttgtcacctggacacactcatttgttagccatagtgggttcccttggtcagcaacagtgcatgtacctctggatgtcatctgaggtgagaccaccgaggccttttctctctgtgtacagaggggagttaggagttgctggactggatgcattacgaggactggggacagggtagagggacatccagggatcagggcatgagtgggggcaaccccccggcctctgccctggcatggtctccgcatgggctgaggtgtagctgattggctgccacatttcggccatgctggctggcgtgcccatgttgcagatattttcccgagttccccagaatggatggtattgaatctcagccacatgcaacactgtgtccagcattctttgcaataaatactttttaaaaaataa

SEQ ID NO: 63 - DNA, 83 bp, Homo sapiens
  source 1..83: mol_type = genomic DNA
ccttggagtaaagtagcagcacataatggtttgtggattttgaaaaggtgcaggccatattgtgctgcctcaaaaatacaagg

SEQ ID NO: 64 - DNA, 84 bp, Homo sapiens
  source 1..84: mol_type = genomic DNA
ctccccatggccctgtctcccaacccttgtaccagtgctgggctcagaccctggtacaggcctgggggacagggacctggggac

SEQ ID NO: 65 - DNA, 1759 bp, Homo sapiens
  source 1..1759: mol_type = genomic DNA
ggagcctccctgcaccaagctggccctgcacggctgcaagggaggctcctgtggacaggccaggcaggtgggcctcaggaggtgcctccaggcggccagtgggcctgaggccccagcaagggctagggtccatctccagtcccaggacacagcagcggccaccatggccacgcctgggctccagcagcatcagagcagcccctgtggttggcagcaaagttcagcttggctgggcccgctgtgaggggcttcgcgctacgccctgcggtgtcccgagggctgaggtctcctcatcttctccctagcagtggatgagcaacccaacgggggcccggggaggggaactggccccgagggagaggaaccccaaagccacatctgtagccaggatgagcagtgtgaatccaggcagcccccaggaccggggaggcacaggtggcccccaccacccggaggagcagctcctgcccctgtccgggggatgactgattctcctccgccaggccacccagaggagaaggccaccccgcctggaggcacaggccatgaggggctctcaggaggtgctgctgatgtggcttctggtgttggcagtgggcggcacagagcacgcctaccggcccggccgtagggtgtgtgctgtccgggctcacggggaccctgtctccgagtcgttcgtgcagcgtgtgtaccagcccttcctcaccacctgcgacgggcaccgggcctgcagcacctaccgaaccatctataggaccgcctaccgccgcagccctgggctggcccctgccaggcctcgctacgcgtgctgccccggctggaagaggaccagcgggcttcctggggcctgtggagcagcaatatgccagccgccatgccggaacggagggagctgtgtccagcctggccgctgccgctgccctgcaggatggcggggtgacacttgccagtcagatgtggatgaatgcagtgctaggaggggcggctgtccccagcgctgcgtcaacaccgccggcagttactggtgccagtgttgggaggggcacagcctgtctgcagacggtacactctgtgtgcccaagggagggccccccagggtggcccccaacccgacaggagtggacagtgcaatgaaggaagaagtgcagaggctgcagtccagggtggacctgctggaggagaagctgcagctggtgctggccccactgcacagcctggcctcgcaggcactggagcatgggctcccggaccccggcagcctcctggtgcactccttccagcagctcggccgcatcgactccctgagcgagcagatttccttcctggaggagcagctggggtcctgctcctgcaagaaagactcgtgactgcccagcgccccaggctggactgagcccctcacgccgccctgcagcccccatgcccctgcccaacatgctgggggtccagaaaccacctcggggtgactgagcggaaggccaggcagggccttcctcctcttcctcctccccttcctcgggaggctccccagaccctggcatgggatgggctgggatcttctctgtgaatccacccctggctacccccaccctggctaccccaacggcatcccaaggccaggtgggccctcagctgagggaaggtacgagctccctgctggagcctgggacccatggcacaggccaggcagcccggaggctgggtggggcctcagtgggggctgctgcctgacccccagcacaataaaaatgaaacgtga

SEQ ID NO: 66 - DNA, 110 bp, Homo sapiens
  source 1..110: mol_type = genomic DNA
acccggcagtgcctccaggcgcagggcagcccctgcccaccgcacactgcgctgccccagacccactgtgcgtgtgacagcggctgatctgtgcctgggcagcgcgaccc

SEQ ID NO: 67 - DNA, 80 bp, Homo sapiens
  source 1..80: mol_type = genomic DNA
tgggatgaggtagtaggttgtatagttttagggtcacacccaccactgggagataactatacaatctactgtctttccta

SEQ ID NO: 68 - DNA, 72 bp, Homo sapiens
  source 1..72: mol_type = genomic DNA
aggttgaggtagtaggttgtatagtttagaattacatcaagggagataactgtacagcctcctagctttcct

SEQ ID NO: 69 - DNA, 74 bp, Homo sapiens
  source 1..74: mol_type = genomic DNA
gggtgaggtagtaggttgtatagtttggggctctgccctgctatgggataactatacaatctactgtctttcct

SEQ ID NO: 70 - DNA, 83 bp, Homo sapiens
  source 1..83: mol_type = genomic DNA
cggggtgaggtagtaggttgtgtggtttcagggcagtgatgttgcccctcggaagataactatacaacctactgccttccctg

SEQ ID NO: 71 - DNA, 84 bp, Homo sapiens
  source 1..84: mol_type = genomic DNA
gcatccgggttgaggtagtaggttgtatggtttagagttacaccctgggagttaactgtacaaccttctagctttccttggagc

SEQ ID NO: 72 - DNA, 87 bp, Homo sapiens
  source 1..87: mol_type = genomic DNA
cctaggaagaggtagtaggttgcatagttttagggcagggattttgcccacaaggaggtaactatacgacctgctgcctttcttagg

SEQ ID NO: 73 - DNA, 84 bp, Homo sapiens
  source 1..84: mol_type = genomic DNA
aggctgaggtagtagtttgtacagtttgagggtctatgataccacccggtacaggagataactgtacaggccactgccttgcca

SEQ ID NO: 74 - DNA, 89 bp, Homo sapiens
  source 1..89: mol_type = genomic DNA
accatgctgtagtgtgtgtaaacatcctacactctcagctgtgagctcaaggtggctgggagagggttgtttactccttctgccatgga

SEQ ID NO: 75 - DNA, 90 bp, Homo sapiens
  source 1..90: mol_type = genomic DNA
ccgggcccctgtgagcatcttaccggacagtgctggatttcccagcttgactctaacactgtctggtaacgatgttcaaaggtgacccgc

SEQ ID NO: 76 - DNA, 110 bp, Homo sapiens
  source 1..110: mol_type = genomic DNA
ggccagctgtgagtgtttctttggcagtgtcttagctggttgttgtgagcaatagtaaggaagcaatcagcaagtatactgccctagaagtgctgcacgttgtggggccc

SEQ ID NO: 77 - DNA, 87 bp, Homo sapiens
  source 1..87: mol_type = genomic DNA
accgcagggaaaatgagggacttttgggggcagatgtgtttccattccactatcataatgcccctaaaaatccttattgctcttgca

SEQ ID NO: 78 - DNA, 110 bp, Homo sapiens
  source 1..110: mol_type = genomic DNA
aaagatcctcagacaatccatgtgcttctcttgtccttcattccaccggagtctgtctcatacccaaccagatttcagtggagtgaagttcaggaggcatggagctgaca

SEQ ID NO: 79 - DNA, 112 bp, Homo sapiens
  source 1..112: mol_type = genomic DNA
ccaccccggtcctgctcccgccccagcagcacactgtggtttgtacggcactgtggccacgtccaaaccacactgtggtgttagagcgagggtgggggaggcaccgccgagg

SEQ ID NO: 80 - DNA, 2424 bp, Homo sapiens
  source 1..2424: mol_type = genomic DNA
ctggccgcagtcgcggcagtggtggcttcccatccccaaaaggcgccctccgactccttgcgccgcactgctcgccgggccagtccggaaacgggtcgtggagctccgcaccactcccgctggttcccgaaggcagatcccttctcccgagagttgcgagaaactttcccttgtccccgacgctgcagcggctcgggtaccgtggcagccgcaggtttctgaaccccgggccacgctccccgcgcctcggcttcgcgctcgtgtagatcgttccctctctggttgcacgctggggatcccggacctcgattctgcgggcgagatgcccctgggacacatcatgaggctggacctggagaaaattgccctggagtacatcgtgccctgtctgcacgaggcaatggtggcttgctatccgggaaatggaacaggttatgttcgccacgtggacaaccccaacggtgatggtcgctgcatcacctgcatctactatctgaacaagaattgggatgccaagctacatggtgggatcctgcggatatttccagaggggaaatcattcatagcagatgtggagcccatttttgacagactcctgttcttctggtcagatcgtaggaacccacacgaagtgcagccctcttacgcaaccagatatgctatgactgtctggtactttgatgctgaagaaagggcagaagccaaaaagaaattcaggaatttaactaggaaaactgaatctgccctcactgaagactgaccgtgctctgaaatctgctggccttgttcattttagtaacggttcctgaattctcttaaattctttgagatccaaagatggcctcttcagtgacaacaatctccctgctacttcttgcatccttcacatccctgtcttgtgtgtggtacttcatgttttcttgccaagactgtgttgatcttcagatactctctttgccagatgaagttacttgctaactccagaaattcctgcagacatcctactcggccagcggtttacctgatagattcggtaatactatcaagagaagagcctaggagcacagcgagggaatgaaccttacttgcactttatgtatacttcctgatttgaaaggaggaggtttgaaaagaaaaaaatggaggtggtagatgccacagagaggcatcacggaagccttaacagcaggaaacagagaaatttgtgtcatctgaacaatttccagatgttcttaatccagggctgttggggtttctggagaattatcacaacctaatgacattaatacctctagaaagggctgctgtcatagtgaacaatttataagtgtcccatggggcagacactccttttttcccagtcctgcaacctggattttctgcctcagccccattttgctgaaaataatgactttctgaataaagatggcaacacaattttttctccattttcagttcttacctgggaacctaattccccagaagctaaaaaactagacattagttgttttggttgctttgttggaatggaatttaaatttaaatgaaaggaaaaatatatccctggtagttttgtgttaaccactgataactgtggaaagagctaggtctactgatatacaataaacatgtgtgcatcttgaacaatttgagaggggaggtggagttggaaatgtgggtgttcctgttttttttttttttttttttttagttttcctttttaatgagctcaccctttaacacaaaaaaagcaaggtgatgtattttaaaaaaggaagtggaaataaaaaaatctcaaagctatttgagttctcgtctgtccctagcagtctttcttcagctcacttggctctctagatccactgtggttggcagtatgaccagaatcatggaatttgctagaactgtggaagcttctactcctgcagtaagcacagatcgcactgcctcaataacttggtattgagcacgtattttgcaaaagctacttttcctagttttcagtattactttcatgttttaaaaatccctttaatttcttgcttgaaaatcccatgaacattaaagagccagaaatattttcctttgttatgtacggatatatatatatatagtcttccaagatagaagtttactttttcctcttctggttttggaaaatttccagataagacatgtcaccattaattctcaacgactgctctattttgttgtacggtaatagttatcaccttctaaattactatgtaatttattcacttattatgtttattgtcttgtatcctttctctggagtgtaagcacaatgaagacaggaattttgtatatttttaaccaatgcaacatactctcagcacctaaaatagtgccgggaacatagtaagggctcagtaaatacttgttgaataaactcagtctcctaca

SEQ ID NO: 81 - DNA, 14167 bp, Homo sapiens
  source 1..14167: mol_type = genomic DNA
actcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgagtatgttaagctctttatgactgtttttgtagtggtatagagtactgcagaatacagtaagctgctctattgtagcatttcttgatgttgcttagtcacttatttcataaacaacttaatgttctgaataatttcttactaaacattttgttattgggcaagtgattgaaaatagtaaatgctttgtgtgattgaatctgattggacattttcttcagagagctaaattacaattgtcatttataaaaccatcaaaaatattccatccatatactttggggacttgtagggatgcctttctagtcctattctattgcagttatagaaaatctagtcttttgccccagttacttaaaaataaaatattaacactttcccaagggaaacactcggctttctatagaaaattgcactttttgtcgagtaatcctctgcagtgatacttctggtagatgtcacccagtggtttttgttaggtcaaatgttcctgtatagtttttgcaaatagagctgtatactgtttaaatgtagcaggtgaactgaactggggtttgctcacctgcacagtaaaggcaaacttcaacagcaaaactgcaaaaaggtggtttttgcagtaggagaaaggaggatgtttatttgcagggcgccaagcaaggagaattgggcagctcatgcttgagacccaatctccatgatgacctacaagctagagtatttaaaggcagtggtaaatttcaggaaagcagaagttaaaggcaaaattgtaaatcagtcgagatcgggtgccttcagggtggtatggctgtataccaaaattgtaaatcactacatgaagcttatatattggtttggcctgaaaggtgaagtggggtaggcagggggcgggcttacaggttatggtggattcaaagactccctgatttgtgattggttaaggaagcaaagctttgtctaaaaacttggggtccgcagaaaggaacattaaggtctggccaggcccctcaggaagaaactgagagcaaagaatggaggtcagagtttagtccctggtgttcccccttatctgacgtctgtgtgaatccatttggtgggggtctgggtttctgaaaagtagctcaggggcacgtgttaaggatgtctctaggtgactctaacttccctggctattgtttgaaactgttatgaccttcttgcttatcagcttgctggtttccttctcggggcgagctgggtgcctggagttttcggtgaaggaaactcaagattctcctttatttctgtgcttgtgggaatccccctggcacaccccaaagaggggtccctgctccgtctcacagggatctttttgtatatttggcttagcatcatacatttgccatgttgtttcatcatctgcctaatttactgtttttgaatatttcatttgtttctaattgttactacagataatgctggggtgagcaactctgtgtacataggtttatctcctattggaatattttctttatataggcgttttttttttttctttttttttggagacagagtcttgctctgttgcccaggctggagtgcagtggcgcgaccggagctcactgcaacctccacttcccgggttcaagtgattgtcccacctcagcctcctgaatagctgggattacaggtgcatgctaccatgcctggctactttttgtatttttagcagagacagggtttcaccatgttggccagggtggtctcgaactcctgacctcaagtgatccgtctggctcagcctcccaaagtgctgggattacaggtgtgagccactgcacctggcctatataggcttttttcttaaacctatttagtaatgttttcccaagtttattttttatttttaattttttccccaagtttatttttctatttttttttcatggaaaaatggggtaacttagcagtttcaatattgaagactgaagtttaaaaaaaatttaaattcaaggtacttttaaaattcagttagaaaagtaggctttaaaaattattagagacaagagtaccaaagcggtgtgtgtatgtgtgtgtgtgtatgcatgcttgtggattggaaaaactttggagactgattacttttcattatatatgtgtcacagtgaaacagcttttatgtgtcatgtaagattactgcttgcctctctaaggaaggtcgtgactgtttaaatagacgggcaaggtggaaccttttgaaagatgagcttttgaatataagttgtctgctagatcatggtttgtattgaactaacaaggtttgcagatctgctgacttatataaagctttttgattcctactaagctttaagatttaaaaaatgttcaatgttgaaatttctgtggggctctatttttgctttggctttctggtgagagagtgaggaagcattctttccttcactaagtttgtctttcttgtcttctggatagattgattttaagagactaagggaatttacaaactaaagattttagtcatctggtggaaaaggagactttaagattgtttagggctgggcggggtgactcacatctgtaatcccagcactttgggaggccaaggcaggcagaacacttgaaggagttcaagaccagcgtggccaacgtggtgaaaccctgtctctactaaaaatacaaaaattgtttagctctgtttttcataatagaaatagaaaaggtaaaattgcttttcttctgaaaagaacaagtattgttcatccaagaagggtttttgtgactgaatcagcagtgcctgccctagtcatagctgtgcttcaaaaacctcagcatgattagtgttggagcaaaacaaggaagcaaagcaaatactgtttttgaaattctatctgttgcttgaactattttgtaataattaaactttgatgttgagaaatcacaactttattgtacacttcattgcaacttgaaattcatggtcttaaagtgagatttgaatttctattgagcgcctttaaaaaagtaataccaaaccataaagttaaaatctatgtatattgagtcatatctaaaaccacgtataaacataaattgtatttcctgttttaattccaggggaagtactgtttgggaaagctattattaggtaaatgttttacaaattactgtttctcactttcagtcataccctaatgatcccagcaagataatgtcctgtcttctaagatgtgcatcaagcctggtacatactgaaaaccctataaggtcctggataatttttgtttgattattcattgaagaaacatttattttccaattgtgtgaagtttttgactgttaataaaagaatctgtcaaccatcaaagaggtctgcattatgcttgcatgtcaaaaactttaaaaatcctataatcttctgtcattttcactgagtttccatgggaaaggaatagtaaataatgggtagttgaaatattactcttaagaccaagacctgtatctccagtcatatctgtaataacatcatctgataacctaaaagcatagtattagggatatacgacaaaaccaaagtgtttttgctgttgtcacataccactcaatacttttacaccaggttgtccagtggacaccagctggatgtcctataattcaattctgacactgtctacctggaattggagtcagatcccacaggtggagggctcagcccacaagactgcccccactccagataccaatcgcaagtccctatacttctgactggctataaagtggggttcccgtgaccctttccttggttttgatttatttgctagagcagctcccagaacacagagaaacactctacttatgtttacccatttttaattttattactttattaaaactacatattggttacaatactaatttattgtaaaggatatttcatttatttatttttttatttagagatggagtctcactgtgtcacccaggctggagtgcggtggcacagtctcaactcactgcaacctccacctcctgagttcaagcgattctcatgcctcagcctcccaagtagctgggattacaagtgtgcaccaccatgcccagctaatttttgtatttttagtagagatggggtttcactctgttggccaggctggtctcaaactcctgacctcaagtcatccacccacctcagcctcccaaagtgctgagattacaggtgtgagccattgcctctggcttgtaaaggatatttcaaagaatacagatgaacagccagatggaagagatgcacagggcaaggcatgtgggaaggggtatggagctttcatgctctctccacgtcatcagctacccagaagctcatctggaccccgaccctttggggttttacggaggcttcattatgtcagcattaccattggcaattggcgattaactcaaccttcagcctctctcccctttctggaggttgggggttgaggctgggagtccaaaccctccaatcttgccttggtctttctggtaagcagccccccgtcctgaagctacctagaggctcccagccaccaatcatttcattggcaaaccaaagatacttcaaggggtttaggagccaagagaccaagcaaggattgagacaatatatatatttcttataaataacatgatcacaaatagtattttatatatttctcataatagctggttgtggttactcacacctatcatcccagctactaagaagctaaggcaggaggatcacaggcccaggagtttgcagctgtagtgagctatgattgtgctactgcactccagcctgggtgacagtgtgagaccctgtctcttaaagttcttgtaacatcttagaacattaaccaaccacagattattgccttaactctgaaaaataaagtaatcatacttttacaagatgtattttacataaagttctggtgatattttactctggtcattggaatataggagcttaccacctggtaccactcttcctaaagtggtatactatttggtatagtactggcatactatttggaaagggatgtgaagttggcatgacctgtctagggtgaagtcatgctgtctgctattcattgtgggctttacagtaatgagagtgcttctgttgtcaagatgcttctgggacacgttaaaaaaaatttaagatttctcagtttgtgaaaaggaaggagcacgaatgctttcaagttcctatgacatgctagtcatctttcagtgtgtcccattgttgcacccagtagtctcgatgcccagcgtcacggtgattccaagtttgcctctctgcccactataatttgtgctccatgaagtcccaggacatgaggttcttgttcatttgtgcgtctgggcccatatccaaatattaaacaatgtgacttaatgtatttatcatattgtaaatatgggtatccccaaggctcagaagttaacacagcatatccacagagaggtttgacagcatggctgtggtgactgggaaggccttatgcttgctcctccttctgctgcttgcctttgtgtgggcatggaaagaacattgaattaggtactggaagacctgggtcctggtcttggagattttaccagagcacctgtgggatattggtcaagatgcctggttttagtcctgctcctttgctgtgaaagaggatcttaaggtgccaactctggcattttattttttacttatttatttttaaattatactttaagttctagggtacatgtgtacaacatgcaggtttgttacatgtatacatgtgccatgttggtgtgctgcacctattaactcgtcatttacattaggtatatctcctaatgctatccctcccccttctccccacccctcgacaggccccggtgtgtgatgttccccttcctgtgtccaagtgttctcattgttcaattcccatctatgagtgagaacatgcggtgtttgtttttttgtccttggaatagtttgctgagaaggatggtttccagcttcatccatgtccctataaaggacatgaactcttccttttttatggctgcatagtattccatggtgtatatgtgccacatcttcttaaaccaatctatcattgatggacatttgggttggttccaagtctttgctattgtgaacagtgccacaataaatatacgtgtgcatgtgtctttatagcagcatgatttataatcctttgggtatatacccagtaatgggatggctgggtcaaatggtatttctagttctagatccctgaggaatcgccacactgtcttccacaatggttgaactagtttacagtcccaccaccagtgtaaaagtgttcctatttctccacatcctctccagcacctgttgtttcctgactttttaatgattgacattctaactggtgtgagatggtatctcattgtggttttgatttgcatttctctgatggcccatgatgatgagcatttttttcatgtgtctgttggatgcatggatgtcttcttttgagaagtgtctcttcatatccttcacctactttttgatggggtttgtttttttcttgtaagtttgtttgagttccttgtagattctggatattagccctttgtcagatgagtagattgcaaaaattttctcccattttgtaggttgcctgttcactctgatggtagtttcttttgctgtgcagaagctcttgagtttaatgagatcccatttgtcaattttggcttctgttgccattgcttttggtgttttagacatgaagtccttgcccatgcctgtgtcctgaatggtactgcataggttttcttctagggtttttatggttttaggtctaacatttaagtctttaatccatcttgaattaatttttgtataaggtgtaaggaagggatccagtttcagctttctacatgtggctagccagttttcccagcaccatttattaaatagggaatcctttccccatttcttgtttttgtcaggtttctcaaagatcagatagttgtagatgtgtggtattatttctgagggctctgttctgttccattggtctatatctctgttttggtactagtaccatgctgttttggttactgcagccttgtagtatagtttgaaatcaggtagcgtgatgcctccagctttgttcttttggcttaggatcgttttggcagtgcaggctcttttttggttccacatgaactttaaagtagttttttccaattctatgaagaaagtcatgggtagcttgatggggatggcattgaatctataaattaccttgggcagtatagccattttcacgatattgattcttcctatccatgagcatggaatgttcttccatttgtttgtgtcctcttttatttccttgagcagtggtttgtagttctccttgaagaggtccttcacatcccttgtaagttggattcctaggtattttattctctttgaggcagttgtgaatgggagttcactcatgatttggctctctgtttgtctgttgttggtgtataagaatgcttgtgatttttgcacattgattttgtatcctgagattttgctgaagttgcttatcaaattaaggagattttgggctgagatgatggggttttctaaatataaaatcatgtcatctgcaaacagggacaatttgacttcctcttttccttattgaataccctttatttctttctcctgcctgatggccctggccagaacttccaacactatgttgaataggagtggtgagagagggcatccctgtcttgtgccagttttcaaagggaatgcttccagtttttgcccattcagtctgatattggctgtgggtttgtcatagatagctcttattattttgagatatgtcccatcaatacctaattcattgagagtttttagcatgaagggctgttgaattttgtccaaggccttttctgcatctgttgagataatcatgtggtttttgtctttggttctgtttatatgctggattacttttattgatttgtgtatgttgaaccagccttgcatcccagggatgaagcccacttgatcatggtggataagctttttgatatgctgctggattcagtttgccagtattttattgaggatttttgcattgattttcatcagggatattggtctaaagttctctttttttgttatgtctctgccaggctttggtatcaggatgatgctggcttcataaaatgagttagggaggattccctctttttctattgattggaatagtttcagaaggaatggtaccagctcttccttgtacctctggtagaattcggctgtgaatccatcttgtcctggactttttttggttggtaggctattaattattgcctcaatttcagagcctgttattggtctattcagggattaaacttcttcctggtttagttttgagagggtgtatgtgtccaggaatttatctatttcttctagattttctagtttatttgcatagaggtgtttatagtattctctgtgtactctgtatttctgtgggattggtggtgatatccactttatcattttttattgcatttatttgattcttctctcttttcttgtttattagtcttgctagcggtctatcaattgtgtttatcttttcaaaaaaccagctcctggattcattcattttttgaaggtttttttgtgtctctgtctccttcagttctgctctgatcttagttatttcttgtcttctgctagctgttgaatgtgtttgctcttgcttctctaatatttttaattgtgatgttagagtgtcaattttagatctttcctgctttctcttgtgggcacttagtgctgtaaatttccctctacacactgctttaaatgtgtcccagagattctggtatgttgtgtctttgttctcgttggtttcaaagaacatctttatttctgccttcattttgttatgtacccagtagtcattcaggagcaggttgttcagtttccatgtagctgagtggttttgagtgagtttcttaatcctgagttctagtttgattgcactgtggtctgagacagtatgttataatttctgttcttttacatttgctgaggagtgctttacttccaactatgtggtcaattttggaataggtgcgatgtggtgctgagaagaatgtatattctgttgatttggggtggagagttctgtagatgtctattaggtctgcttggtgcagagctgaattcaattcctggatatccttgttaactttctgtctcgttgatctgtctaatattgacagtggggtgttagagtctcccattgttattgtgtgggagtctaaatctctttgtaggtctctaaggacttggtttatgaatctgggtgctcctgtattgggtgcatatatatttaagatagttagctcttcttgttgaattgatccctttactatcatgtaatggccttctttgtctcttttgatctttgttggtttaaagtctgttttatcagagactaggattgcaaccccggcctttttttttgttttccgtttgcttggtagatcttcctccatccttttattttgagcctatgtgtgtgtgtctctgtacgtgagatgggtttcctgaatacagcaccctgatgggtcttgactctttatccagtgtgccagtctgtgtcttttaattggagcatttagcccatttacatttaaggttaatattgttatgtgtgaatttgatcctgtcactaggatgttcactggttattttgctcgttagttgatgcagtttcttcctagcctcaatggtctttacaatttggcatgtttttgcagtggctgatactggttgttccattccatgtttagtgcttccttcaggagctcttgtaaggcaggcctggtggtgacaaaatctctcagcatttgcttgtttgtaaaggattttatttctccttcacttatgaagcttagtttggctggatatgaaattctgggttgaaaattcttttcttcaagaacgttgaatattgacccccactctcttctggcttgtagagtttctgctgagagatcagctgttagtctgatgggcttccctttgtgggtaacccgacctttctctctggctgcccttaacattttttccttcatttcaactttggtgaatctgacaattatgtgtcttggagttgctcttctcgaggagtatctttgtggcgttctctgtatttcctgaatttgaatgttgtcctcccttgctagattggggaagttctcctggataatatcctgcagagtgttttccagcttggttccattctccccgtcactttcaggtacaccaatcagacgtagatttggtcttttcacatagtcccatatttcttggaagctttgttcgtttctttttactcttttttctcttaacttctcttctcacttcatttcattcatctgatcttcaatcactgataacctttcttccagttgatcgaattggctactgaagcttgtgcattcgtcacgtagttctcatgccatggttttcagctccatcaggtcatttaaggacttctctatgctggttattctagttagccatttgtctaatcttttttcaaggtttttaggttctttgtgatgggttcgaacttcctcttttagcttggagaagtttgatcatctgaagccttcttctctcaactcgtcaaagtcattctccgtccagctttgttctgttgctggcaaggagctatgttcctttggagggggagaggcgctctgatttttagaattttcagcttttctgctctgttttttccccatctttgtggttttatctacctttggtctttggtgatggtgatgtgcagatggggttttggtgtggatgtcctttctgtttgttagttttccttctaacagtcaggaccgccagctgcaggtctgttggagtttgctggacgtccacgccagaccctgtttgcctgggtatcacagtggaggctgtagaacagcgaatattgctgaacagcaaatgttgctgcctgatcattcctctggaagcttcatctcagaggggtacccggccatgtggggtgtcagtctgcccctacttgggggtgcctcccagttaggctactcgggggtcagggactcacttgaggaggcagtctctccgttctcagatctcaaactctgtgctgggagaaccactggtctcttcaaagctgtcagacagggacattgaagtctgcagagatttctgctgccttttgttcggctatgccatgcccccagaggtggagtctacagaggcaggcaggcaggcctccttgagctgcagtgggctccacctagttcgagcttcccggctgctttatttacctactgaagcctcagcaatggtgggctcccctcccctagcctcactgccaccttgcagttcgatctcagactgctgtgctagcaatgagcaaggctccatgggcgtgggaccctccgagctaggcacaggatataatctcctggtgtgccgtttgctaagaccattggaaaaacgcagtattagggtgggagtgacccaattttccaggtgccgtctgtcacagcttcccttggctaggaaagggaattccctgaccccttgccttcccgggtgaggccatgccttgccctgcttcagctcatgctcagtgggctgcacccaccgtcctacacccactgtccaacaagccccagtgagatgaacctggtacctcagttggaagtgcagaaatcacctgtcttctgcgtcgctcacgctgggagctgtagactggagctgttcctattcggccatcttggaacccaacccaagtctggcattttttggttttctctgtgtattgactatgattatagaggataaatggatattgatattattaaatcgtcatttacagaaggcacacaacttatatcctttaaacaaagttctccaaccagtagggtatacttgctggtagtgcttacagctgccaccattcttcactggggctcagagtggatgcctggaggaaaaaaacaaatgaatcttttagctcctagttactgttagcatttttaccagaaaacaatttaaaaaatgacacaagtaagtcttcacctatcaaggctgagtgtggtggcctatacttgtaatctctgtgctttgggaggccaacgcaggaggatctcttgagtccaggagtttgagaccagcctgggcaacaaagcaagaccccatctctacagaaagttaaaatttagctgggcatgctggcacatccctatagtcccagctacttgagaggctgagttgggaggatcccttgaggtcatgagttggagtctacagtgagctgtgattacagtgtgagtctacactgcactccagtctgggtgaaagagatcctgtctctcaaaaaacaagtgattctcctgggaagagatttgtttaaaaagtctgcacctctattgatgataactttgaatgtaaatggattaaattctcccacttaaaagatatagattgactgaatggattaaaaaccaccatcatgactcgactgtatgctgcctacaagaaacctcacctgtaaagacaggccaaaagtgaaggtatggagaaagatattgcatacaaatggaagccaaaaaagagcagaaataactatacttacatgagataaaatagactttaagtcaaaaactgtaaaaagaaaagtcattatgtaatgaaacaaggatcaattcatgcaagatataatcatatttttttattaagaaaatttttttgtagagatggggttatgctatgttgcctggctagtctcaaactctgggtctcaaagcagtcctcctgctttggcctcctaaggtgctgggattataggcatgagccactgtgccaggcctgaggatattatcattataaatatatatgtaccccacactggatgacccagatataaagcaaatattattagatttaaagggagaaatagactctaatacaatagtagttgaggacttcactctactctcagcattggacagattttctagacataaaaccaataaatattggatttaaattgcactttacaccaaatggacctgacatttacagaacattctaataggtgcagaacactctaatagctgcagaacacacgttcttttcatcagcacatggaacattcaccagactagttctggaacccacttcctgccttagtgttctttccaccacagtgcatgataattccgacagaacgccttttatttgtaccttattagttgtagggggaaagctttttcatgtacaaaaatgctgtaattttctgatttaataaagtaattccactga

SEQ ID NO: 82 - DNA, 6719 bp, Homo sapiens
  source 1..6719: mol_type = genomic DNA
aatccatcatggcggccgcggggtttggtgtctgtgcctgagcagcgctggagccggagccggttcccgggtcctgcggctgaggagcccctccgctgtccacggcccctaccggcggggggcggctgggtccctcggcggagctcgggagatgtgactgcctgagggcggtggtggtgtcagcgtccggggccgggggagggggtgtctcgggcagagacccccgggcttggggcagctgaggcggccgggcctcctctacacggggcccgccttccgctgtctgggccgcgagagtccttcgtcccttacagccccgccccggctttgggacactgcgggtggtctgtttcccccagcttgggacaccccgttttctgaggcgtggaagagcgtcgccccggagtaagctgcccgtgccgcgccccgacagcttccctcagccccaagccgccccttattccggatcccggccccaactttggccacggagcctcccattcaaatccctcccttgctgtcaaggggtctccccttcccccaaggtggctcccgcgagcctctaatgccctgacttcttccaatgtcacctacggcccccttagtctcagctcagccaaaaactttaatgcaaaggaaaagtctggattggttccacaggccttttaaaaagcggacttaaaagttgctggcaatgcattccttttcgtcagagtcgagggcaaactcgctgaaatctgggtgacccgtgtccttttccggagagcaaagcagagaagcgagagcggccactagttcggcaggaaatttgttggaagatgaagaagctaagatagggggttggtgacttccacaggaaaagttctggaggagtagccaaagaccatcagcgtttcctttatgtgtgagaattgaaatgactagcattattgacccttttcagcatcccctgtgaatatttctgtttaggtttttcttcttgaaaagaaattgttattcagcccgtttaaaacaaatcaagaaacttttgggtaacattgcaattacatgaaattgataaccgcgaaaataattggaactcctgcttgcaagtgtcaacctaaaaaaagtgcttccttttgttatggaagatgtctttctgtgattgacttcaattgctgacttgtggagatgcagcgaatgtgaaatcccacgtatatgccatttccctctacgctcgctgaccgttctggaagatcttgaaccctcttctggaaaggggtacctattattactttatggggcagcagcctggaaaagtacttggggaccaaagaaggccaagcttgcctgccctgcattttatcaaaggagcagggaagaaggaatcatcgaggcatgggggtccacactgcaatgtttttgtggaacatgaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatctgctgagcagcgggatcaatggcagcttcttggtgcgtgagagtgagagcagtcctggccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctggtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgcctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagctggaaagggcagtgccttagggacccctgctgcagctgagccagtgacccccaccagcaaagcaggctcaggtgcaccagggggcaccagcaagggccccgccgaggagtccagagtgaggaggcacaagcactcctctgagtcgccagggagggacaaggggaaattgtccaggctcaaacctgccccgccgcccccaccagcagcctctgcagggaaggctggaggaaagccctcgcagagcccgagccaggaggcggccggggaggcagtcctgggcgcaaagacaaaagccacgagtctggttgatgctgtgaacagtgacgctgccaagcccagccagccgggagagggcctcaaaaagcccgtgctcccggccactccaaagccacagtccgccaagccgtcggggacccccatcagcccagcccccgttccctccacgttgccatcagcatcctcggccctggcaggggaccagccgtcttccaccgccttcatccctctcatatcaacccgagtgtctcttcggaaaacccgccagcctccagagcggatcgccagcggcgccatcaccaagggcgtggtcctggacagcaccgaggcgctgtgcctcgccatctctaggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgacagcaggcagtggtccagcggccactcaggacttcagcaagctcctcagttcggtgaaggaaatcagtgacatagtgcagaggtagcagcagtcaggggtcaggtgtcaggcccgtcggagctgcctgcagcacatgcgggctcgcccatacccgtgacagtggctgacaagggactagtgagtcagcaccttggcccaggagctctgcgccaggcagagctgagggccctgtggagtccagctctactacctacgtttgcaccgcctgccctcccgcaccttcctcctccccgctccgtctctgtcctcgaattttatctgtggagttcctgctccgtggactgcagtcggcatgccaggacccgccagccccgctcccacctagtgccccagactgagctctccaggccaggtgggaacggctgatgtggactgtctttttcatttttttctctctggagcccctcctcccccggctgggcctccttcttccacttctccaagaatggaagcctgaactgaggccttgtgtgtcaggccctctgcctgcactccctggccttgcccgtcgtgtgctgaagacatgtttcaagaaccgcatttcgggaagggcatgcacgggcatgcacacggctggtcactctgccctctgctgctgcccggggtggggtgcactcgccatttcctcacgtgcaggacagctcttgatttgggtggaaaacagggtgctaaagccaaccagcctttgggtcctgggcaggtgggagctgaaaaggatcgaggcatggggcatgtcctttccatctgtccacatccccagagcccagctcttgctctcttgtgacgtgcactgtgaatcctggcaagaaagcttgagtctcaagggtggcaggtcactgtcactgccgacatccctcccccagcagaatggaggcaggggacaagggaggcagtggctagtggggtgaacagctggtgccaaatagccccagactgggcccaggcaggtctgcaagggcccagagtgaaccgtcctttcacacatctgggtgccctgaaagggcccttcccctcccccactcctctaagacaaagtagattcttacaaggccctttcctttggaacaagacagccttcacttttctgagttcttgaagcatttcaaagccctgcctctgtgtagccgccctgagagagaatagagctgccactgggcacctgcgcacaggtgggaggaaagggcctggccagtcctggtcctggctgcactcttgaactgggcgaatgtcttatttaattaccgtgagtgacatagcctcatgttctgtgggggtcatcagggagggttaggaaaaccacaaacggagcccctgaaagcctcacgtatttcacagagcacgcctgccatcttctccccgaggctgccccaggccggagcccagatacgggggctgtgactctgggcagggacccggggtctcctggaccttgacagagcagctaactccgagagcagtgggcaggtggccgcccctgaggcttcacgccgggagaagccaccttcccaccccttcataccgcctcgtgccagcagcctcgcacaggccctagctttacgctcatcacctaaacttgtactttatttttctgatagaaatggtttcctctggatcgttttatgcggttcttacagcacatcacctctttgcccccgacggctgtgacgcagccggagggaggcactagtcaccgacagcggccttgaagacagagcaaagcgcccacccaggtcccccgactgcctgtctccatgaggtactggtcccttccttttgttaacgtgatgtgccactatattttacacgtatctcttggtatgcatcttttatagacgctcttttctaagtggcgtgtgcatagcgtcctgccctgccccctcgggggcctgtggtggctccccctctgcttctcggggtccagtgcattttgtttctgtatatgattctctgtggttttttttgaatccaaatctgtcctctgtagtattttttaaataaatcagtgtttacattagaa

SEQ ID NO: 83 - DNA, 1441 bp, Homo sapiens
  source 1..1441: mol_type = genomic DNA
atccagatttgcttttacattttcttgcctgagtctgaggtgaacagtgaacatatttacatttgatttaacagtgaaccttaattctttctggcttcacagtgaaacaagtttatgcaatcgatcaaatattttcatccctgaggttaacaattaccatcaaaatgttttgtggagactatgtgcaaggaaccatcttcccagctcccaatttcaatcccataatggatgcccaaatgctaggaggagcactccaaggatttgactgtgacaaagacatgctgatcaacattctgactcagcgctgcaatgcacaaaggatgatgattgcagaggcataccagagcatgtatggccgggacctgattggggatatgagggagcagctttcggatcacttcaaagatgtgatggctggcctcatgtacccaccaccactgtatgatgctcatgagctctggcatgccatgaagggagtaggcactgatgagaattgcctcattgaaatactagcttcaagaacaaatggagaaattttccagatgcgagaagcctactgcttgcaatacagcaataacctccaagaggacatttattcagagacctcaggacacttcagagatactctcatgaacttggtccaggggaccagagaggaaggatatacagaccctgcgatggctgctcaggatgcaatggtcctatgggaagcctgtcagcagaagacgggggagcacaaaaccatgctgcaaatgatcctgtgcaacaagagctaccagcagctgcggctggttttccaggaatttcaaaatatttctgggcaagatatggtagatgccattaatgaatgttatgatggatactttcaggagctgctggttgcaattgttctctgtgttcgagacaaaccagcctattttgcttatagattatatagtgcaattcatgactttggtttccataataaaactgtaatcaggattctcattgccagaagtgaaatagacctgctgaccataaggaaacgatacaaagagcgatatggaaaatccctatttcatgatatcagaaattttgcttcagggcattataagaaagcactgcttgccatctgtgctggtgatgctgaggactactaaaatgaagaggacttggagtactgtgcactcctctttctagacacttccaaatagagattttctcacaaatttgtactgttcatggcactattaacaaaactatacaatcatattttctcttctatctttgaaattattctaagccaaagaaaactatgaatgaaagtatatgatactgaatttgcctactatcctgaatttgcctactatctaatcagcaattaaataaattgtgcatgatggaataatagaaaaattgcattggaatagattttatttaaatgtgaaccatcaacaaccta

SEQ ID NO: 84 - DNA, 2028 bp, Homo sapiens
  source 1..2028: mol_type = genomic DNA
gcggagcgggccctaccgtgtgcgcagaaagaggaggcgcttgccttcagcttgtgggaaatcccgaagatggccaaagacaactcaactgttcgttgcttccagggcctgctgatttttggaaatgtgattattggttgttgcggcattgccctgactgcggagtgcatcttctttgtatctgaccaacacagcctctacccactgcttgaagccaccgacaacgatgacatctatggggctgcctggatcggcatatttgtgggcatctgcctcttctgcctgtctgttctaggcattgtaggcatcatgaagtccagcaggaaaattcttctggcgtatttcattctgatgtttatagtatatgcctttgaagtggcatcttgtatcacagcagcaacacaacaagactttttcacacccaacctcttcctgaagcagatgctagagaggtaccaaaacaacagccctccaaacaatgatgaccagtggaaaaacaatggagtcaccaaaacctgggacaggctcatgctccaggacaattgctgtggcgtaaatggtccatcagactggcaaaaatacacatctgccttccggactgagaataatgatgctgactatccctggcctcgtcaatgctgtgttatgaacaatcttaaagaacctctcaacctggaggcttgtaaactaggcgtgcctggtttttatcacaatcagggctgctatgaactgatctctggtccaatgaaccgacacgcctggggggttgcctggtttggatttgccattctctgctggactttttgggttctcctgggtaccatgttctactggagcagaattgaatattaagcataaagtgttgccaccatacctccttccccgagtgactctggatttggtgctggaaccagctctctcctaatattccacgtttgtgccccacactaacgtgtgtgtcttacattgccaagtcagatggtacggacttcctttaggatctcaggcttctgcagttctcatgactcctacttttcatcctagtctagcattctgcaacatttatatagactgttgaaaggagaatttgaaaaatgcataataactacttccatccctgcttatttttaatttgggaaaataaatacattcgaaggaacctgtgttatcacagtaacccagagctgtatttggctagcaatctgcctgtatctctcactattatctaaaagaaaccttccaatgcttctgttgatctcagtattgtcaggggaacagagaagttgggaaaagattactgaaatataccttttgcatttctttctagagtagctcccatatatggagatgggtgattctcttgatgccaccttcagatccttttattctccagaataattcttaacagtggttcaaatttcctttcataccttgaagtatgtgtttagtagcctcaattctccattaattaaaagtgtgggctgggcgtgggggctcatgcctgtaatcccagcactttgggaggccgaggtgggcagatcacctgaggtcaggagttcaagaccagcctggccaacatggtgaaaccccgtctctacaaaaatacaaaaattagccaggcgtgatggcaggtgcctgtaatcctagctacttggcaggctaacgcaggagaatcacttgaccgggagacagaggttgcagtgagctgagatcgtacctattgcactccatcctggatgaaagagccagactctgtctcaaaacaaacaaaaaagcgtggggacttctggggacagacaaggtgcctgttatatatttactcagtctttgccctgaatggtctcagcttgagaccatttcaaactggagagaagcaagccagccaatagaatggggtgatttacagggatttctgtttactgtcaaaatatttctcatctgcactatgtttccatttgtggtcctgaaggaaattcttataactcaacatttgtctggtcttataagtaaagacagctttaaaatctg

SEQ ID NO: 85 - DNA, 1288 bp, Homo sapiens
  source 1..1288: mol_type = genomic DNA
gagaaactcagagaccaagtccattgagagactgaggggaaagagaggagagaaagaaaaagagagtgggaacagtaaagagaaaggaagacaacctccagagaaagcccccggagacgtctctctgcagagaggcggcagcacccggctcacctgcgaagcgcctgggaagcgagtgcccctaacatgcggctgccgctgcttgtgtccgcgggagtcctgctggtggctctcctgccctgcccgccatgcagggcgctcctgagccgcgggccggtcccgggagctcggcaggcgccgcagcaccctcagcccttggatttcttccagccgccgccgcagtccgagcagccccagcagccgcaggctcggccggtcctgctccgcatgggagaggagtacttcctccgcctggggaacctcaacaagagcccggccgctcccctttcgcccgcctcctcgctcctcgccggaggcagcggcagccgcccttcgccggaacaggcgaccgccaactttttccgcgtgttgctgcagcagctgctgctgcctcggcgctcgctcgacagccccgcggctctcgcggagcgcggcgctaggaatgccctcggcggccaccaggaggcaccggagagagaaaggcggtccgaggagcctcccatctccctggatctcaccttccacctcctccgggaagtcttggaaatggccagggccgagcagttagcacagcaagctcacagcaacaggaaactcatggagattattgggaaataaaacggtgcgtttggccaaaaagaatctgcatttagcacaaaaaaaatttaaaaaaatacagtattctgtaccatagcgctgctcttatgccatttgtttatttttatatagcttgaaacatagagggagagagggagagagcctataccccttacttagcatgcacaaagtgtattcacgtgcagcagcaacacaatgttattcgttttgtctacgtttagtttccgtttccaggtgtttatagtggtgttttaaagagaatgtagacctgtgagaaaacgttttgtttgaaaaagcagacagaagtcactcaattgtttttgttgtggtctgagccaaagagaatgccattctcttgggtgggtaagactaaatctgtaagctctttgaaacaactttctcttgtaaacgtttcagtaataaaacatctttccagtccttggtcagtttggttgtgtaagagaatgttgaatacttatatttttaataaaagttgcaaaggtaatcatga

SEQ ID NO: 86 - DNA, 5580 bp, Homo sapiens
  source 1..5580: mol_type = genomic DNA
gcctgctctccggcggagctgcgtgaggcccggccggccccggccccccccttccggccgcccccgcctcctggcccacgcctgcccgcgctctgcccaccagcgcctccatcgggcaaggcggccccgcgtcgacgccgcccgctgcctcgctgctgactcccgtcccgggcgccgtccgcggggtcgcgctccgccgggcctgcggattccccgccgcctcctcttcatctacctcaactccccccatccccgcttcgcccgaggaggcggttccccccgcaggcagtccggctcgcaggccgccggcgttgtcacccccccccgcgctccccctccagccctccccccggcgcgcagcctcgggccgctcccctttccgcgctgcgtcccggagcggccccggtgccgccaccgcctgtccccctcccgaggcccgggctcgcgacggcagagggctccgtcggcccaaaccgagctgggcgcccgcggtccgggtgcagcctccactccgccccccagtcaccgcctcccccggcccctcgacgtggcgcccttccctccgcttctctgtgctccccgcgcccctcttggcgtctggccccggcccccgctctttctcccgcaaccttcccttcgctccctcccgtcccccccagctcctagcctccgactccctccccccctcacgcccgccctctcgccttcgccgaaccaaagtggattaattacacgctttctgtttctctccgtgctgttctctcccgctgtgcgcctgcccgcctctcgctgtcctctctccccctcgccctctcttcggcccccccctttcacgttcactctgtctctcccactatctctgcccccctctatccttgatacaacagctgacctcatttcccgataccttttcccccccgaaaagtacaacatctggcccgccccagcccgaagacagcccgtcctccctggacaatcagacgaattctccccccccccccaaaaaaaagccatccccccgctctgccccgtcgcacattcggcccccgcgactcggccagagcggcgctggcagaggagtgtccggcaggagggccaacgcccgctgttcggtttgcgacacgcagcagggaggtgggcggcagcgtcgccggcttccagacaccaatgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaagtgagcaaaactgccgcaagtctgcagcccggcgccaccatcctgcagcctcctcctgaccacggacgtttccatcaggttccatcccgaaaatctctcggttccacgtccccctggggcttctcctgacccagtccccgtgccccgcctccccgaaacaggctactctcctcggccccctccatcgggctgaggaagcacagcagcatcttcaaacatgtacaaaatcgattggctttaaacacccttcacataccctccccccaaattatccccaattatccccacacataaaaaatcaaaacattaaactaacccccttcccccccccccacaacaaccctcttaaaactaattggctttttagaaacaccccacaaaagctcagaaattggctttaaaaaaaacaaccaccaaaaaaaatcaattggctaaaaaaaaaaagtattaaaaacgaattggctgagaaacaattggcaaaataaaggaatttggcactccccacccccctctttctcttctcccttggactttgagtcaaattggcctggacttgagtccctgaaccagcaaagagaaaagaaggaccccagaaatcacaggtgggcacgtcgctgctaccgccatctcccttctcacgggaattttcagggtaaactggccatccgaaaatagcaacaacccagactggctcctcactcccttttccatcactaaaaatcacagagcagtcagagggacccagtaagaccaaaggaggggaggacagagcatgaaaaccaaaatccatgcaaatgaaatgtaattggcacgaccctcacccccaaatcttacatctcaattcccatcctaaaaagcactcatactttatgcatccccgcagctacacacacacaacacacagcacacgcatgaacacagcacacacacgagcacagcacacacacaaacgcacagcacacacagcacacagatgagcacacagcacacacacaaacgcacagcacacacacgcacacacatgcacacacagcacacaaacgcacggcacacacacgcacacacatgcacacacagcacacacacaaacgcacagcacacacaaacgcacagcacacacgcacacacagcacacacacgagcacacagcacacaaacgcacagcacacgcacacacatgcacacacagcacacacactagcacacagcacacacacaaagacacagcacacacatgcacacacagcacacacacgcgaacacagcacacacgaacacagcacacacagcacacacacaaacacagcacacacatgcacacagcacacgcacacacagcacacacatgaacacagcacacagcacacacatgcacacacagcacacacgcatgcacagcacacatgaacacagcacacacacaaacacacagcacacacatgcacacacagcacacacactcatgcgcagcacatacatgaacacagctcacagcacacaaacacgcagcacacacgttgcacacgcaagcacccacctgcacacacacatgcgcacacacacgcacacccccacaaaattggatgaaaacaataagcatatctaagcaactacgatatctgtatggatcaggccaaagtcccgctaagattctccaatgttttcatggtctgagccccgctcctgttcccatctccactgcccctcggccctgtctgtgccctgcctctcagaggagggggctcagatggtgcggcctgagtgtgcggccggcggcatttgggatacacccgtagggtgggcggggtgtgtcccaggcctaattccatctttccaccatgacagagatgcccttgtgaggctggcctccttggcgcctgtccccacggcccccgcagcgtgagccacgatgctccccataccccacccattcccgatacaccttacttactgtgtgttggcccagccagagtgaggaaggagtttggccacattggagatggcggtagctgagcagacatgcccccacgagtagcctgactccctggtgtgctcctggaaggaagatcttggggacccccccaccggagcacacctagggatcatctttgcccgtctcctggggaccccccaagaaatgtggagtcctcgggggccgtgcactgatgcggggagtgtgggaagtctggcggttggaggggtgggtggggggcagtgggggctgggcggggggagttctggggtaggaagtggtcccgggagattttggatggaaaagtcaggaggattgacagcagacttgcagaattacatagagaaattaggaacccccaaatttcatgtcaattgatctattccccctctttgtttcttggggcatttttccttttttttttttttttgttttttttttacccctccttagctttatgcgctcagaaaccaaattaaaccccccccccatgtaacaggggggcagtgacaaaagcaagaacgcacgaagccagcctggagaccaccacgtcctgccccccgccatttatcgccctgattggattttgtttttcatctgtccctgttgcttgggttgagttgagggtggagcctcctggggggcactggccactgagcccccttggagaagtcagaggggagtggagaaggccactgtccggcctggcttctggggacagtggctggtccccagaagtcctgagggcggaggggggggttgggcagggtctcctcaggtgtcaggagggtgctcggaggccacaggagggggctcctggctggcctgaggctggccggaggggaaggggctagcaggtgtgtaaacagagggttccatcaggctggggcagggtggccgccttccgcacacttgaggaaccctcccctctccctcggtgacatcttgcccgcccctcagcaccctgccttgtctccaggaggtccgaagctctgtgggacctcttgggggcaaggtggggtgaggccggggagtagggaggtcaggcgggtctgagcccacagagcaggagagctgccaggtctgcccatcgaccaggttgcttgggccccggagcccacgggtctggtgatgccatagcagccaccaccgcggcgcctagggctgcggcagggactcggcctctgggaggtttacctcgcccccacttgtgcccccagctcagcccccctgcacgcagcccgactagcagtctagaggcctgaggcttctgggtcctggtgacggggctggcatgaccccgggggtcgtccatgccagtccgcctcagtcgcagagggtccctcggcaagcgccctgtgagtgggccattcggaacattggacagaagcccaaagagccaaattgtcacaattgtggaacccacattggcctgagatccaaaacgcttcgaggcaccccaaattacctgcccattcgtcaggacacccacccacccagtgttatattctgcctcgccggagtgggtgttcccgggggcacttgccgaccagccccttgcgtccccaggtttgcagctctcccctgggccactaaccatcctggcccgggctgcctgtctgacctccgtgcctagtcgtggctctccatcttgtctcctccccgtgtccccaatgtcttcagtggggggccccctcttgggtcccctcctctgccatcacctgaagacccccacgccaaacactgaatgtcacctgtgcctgccgcctcggtccaccttgcggcccgtgtttgactcaactcaactcctttaacgctaatatttccggcaaaatcccatgcttgggttttgtctttaaccttgtaacgcttgcaatcccaataaagcattaaaagtcatga

SEQ ID NO: 87 - DNA, 5015 bp, Homo sapiens
  source 1..5015: mol_type = genomic DNA
atgcggctcgggctatgacagcctccgtgctcctccacccccgctggatcgagcccaccgtcatgtttctctacgacaacggcggcggcctggtggccgacgagctcaacaagaacatggaaggggcggcggcggctgcagcagcggctgcagcggcggcggctgccggggccgggggcgggggcttcccccacccggcggctgcggcggcagggggcaacttctcggtggcggcggcggccgcggctgcggcggcggccgcggccaaccagtgccgcaacctgatggcgcacccggcgcccttggcgccaggagccgcgtccgcctacagcagcgcccccggggaggcgcccccgtcggctgccgccgctgctgccgcggctgccgctgcagccgccgccgccgccgccgcgtcgtcctcgggaggtcccggcccggcgggcccggcgggcgcagaggccgccaagcaatgcagcccctgctcggcagcggcgcagagctcgtcggggcccgcggcgctgccctatggctacttcggcagcggctactacccgtgcgcccgcatgggcccgcaccccaacgccatcaagtcgtgcgcgcagcccgcctcggccgccgccgccgccgccttcgcggacaagtacatggataccgccggcccagctgccgaggagttcagctcccgcgctaaggagttcgccttctaccaccagggctacgcagccgggccttaccaccaccatcagcccatgcctggctacctggatatgccagtggtgccgggcctcgggggccccggcgagtcgcgccacgaacccttgggtcttcccatggaaagctaccagccctgggcgctgcccaacggctggaacggccaaatgtactgccccaaagagcaggcgcagcctccccacctctggaagtccactctgcccgacgtggtctcccatccctcggatgccagctcctataggagggggagaaagaagcgcgtgccttataccaaggtgcaattaaaagaacttgaacgggaatacgccacgaataaattcattactaaggacaaacggaggcggatatcagccacgacgaatctctctgagcggcaggtcacaatctggttccagaacaggagggttaaagagaaaaaagtcatcaacaaactgaaaaccactagttaatggattaaaaatagagcaagaaggcaacttgaagaaacgcttcagaactcgttgctttgcccagataatgataataatgcttaataataattgaagaatgggaaagagaaagagacagagactggcattttcctctcccgaaggagatctctttctctttaatggaatctacaactgttttaaaactttaagaaaggtaaagactgccagttcttccgccaaccccatcagcccagcccgttaaatgtcaaacgtcaacccccaaaatacgcaatttcagataagttacgcagttactgaaatcttgtaagtatttaagtgatcgttacattttaggacactgcgttagatggtaataatctggaagttggttacaaacgcaagaggccattgtaaacatctgcttgtccttcttaggtcgccattccctttgcatgttaagcgtctgctcaggtaaatcttagtgaaattcctaccgttgttgtacgttctgcaaaacattttatgtatagatttagaggggaaacgagaaggtactgaaataatgatcttggaatatttgctgtgaagggagaaagggagagaaaactcttctgaggatcatttgtcttggtagtatagtaaaaccaaccagctgaacctttcaggctacaagagaacccgggtcggtaatgtctttttaagaataatttttaattgcttataacaagcatattttgtggcatttgaactatatttactgctccaatatccgttattttccaaaggattttgtatctttttgaaaatgtttacatcatcagatgatccacagaattcactttatgtgagatctcccgagagtttccatcccaacatgatggactttggtttgaacacaattcgttttttcatttgaattggcatttcccaatatttgctaaacatttgctggagaaatcatttttcttttttcttttttagaaaactcagaatgaaaattcattcccctgaaatatttaggtgtctatattctatattttgatctattaagggattagtatttttccatgtttattgtgttatcagagtgcattagaaagattagtgattcatcttcacagcacatttttaatcaagcagttatttcaaccagcacattcgttttgttcatattcactatagaatgatatcttgtaaataaagacattcagcacactgtgaaaatgtatttgtgcacctgctttttaaatatttctactaaaaatgaaaaaaaaaaacccttagacctgtagatagtgatatcgtaatattaattgttaataaaatagtcactgccattaaaatctgcagaaaatactctttactttcctatgctaaatgtgcactgcacagaaagaaaaggagcaggtttatcttatataaaccaaggaccattctttattattattcagtacttcttttgctaccaaatttttaagaaaagggagaaatccctcataactaaaatcctaaaaaatgataagtgcttcaaggagattaaagtgtcaggtattacaaaagggagagaaaagaaaggaggaagagatagtatgaattcccaggctccctgtggaaaatgaaaaccagttgttttgtagcagcaatgcctaggctgtcgtattttaaaatctgctgttgggtctgcatgtgtctgagagggcaggttttgatatttgttgaaatgacaggactacgaaaggccttaaaccttgaccttgacgaaaaaaaagacaatttgtgaccttgacttttgacagctcatgaattggccttagctggattagtagatcaagggcgccacctcgcgttgacaagcctccttgtaattcttcaaccaagggaatcaagagctcttacctccttgactttagagattcagtactcaattatttggctccagctggagcaaatttgaatatatgtaaattatacatgtccctgtatatatgtaaatatatataatatgtgtacacatgtatacatttgcatattagtgcttgtgtgtgcttgtatatatatttgtagaatctgtacagatatcatatcattaattgctaatgacgcaagatattgtaatttaaggtgatttctagaattgaggtacaaaatgtactttacaagatgaagccttgttccttttctctaatggccaaatttgatttgtcttcacagtggcttgctctgcatcagatttctgcagatctgctttaaagctgtacatttttgttacagtctaagatgtgttcttaaatcaccattccttcctggtcctcaccctccagggtggtctcacactgtaattagagctattgaggagtctttacagcaaattaagattcagatgccttgctaagtctagagttctagagttatgtttcagaaagtctaagaaacccacctcttgagaggtcagtaaagaggacttaatatttcatatctacaaaatgaccacaggattggatacagaacgagagttatcctggataactcagagctgagtactgctccagggtggtgtgcaatcttatattgatgcttgtgaatctgccatttgatttgtaggataaataaatatgtttaatattaacaacttccatcaaaactataataataatattatatctactgttgacctctaacaacaatcaggtgctgtattcagagtcataaagaatacatatgtatgtttgtatatttaatatacacataggataatgtgtttttgtcaaatctatgctatgcgcatatgtattaaaatgttggtggaatcagtagccgtgggatgcagaattgtgagatgcaggctaattttatctttacgttagaggaaagtaatagtgaatggaagaaccagattcatcattttatggaaaataatattgattggttacaaaagtggctgccggaaacctctccaacctctcctgaggactcacaacttctttatagatgagaattacgaaagagtctctaaagctgtctcaaaaacaggctggtgttgaaacatacatatcgaaaggtctcgagaaattcaattatattttgctgccctgaggattttcactccacttgtggaaacaaatgataatgttgcttcagacccacctctccacctcttgttccaaacagtactccatttccaaatgagctgccatcaagccaaagtaacaactttctcccaaaatgtttaaagtggtggaaaatcccctcaaagctggatcaaactgtgtgtttaagaacagctccaaaagggactcttctgttgacaggggatatttttctttagatgtaagaaatattctgagcctcgagcctaggaaggaggccaacagcagctctccagccgggatcccgcccctttcgaaagccatcaccactacaggaaccctgggtaaagtagtagccttctttttttctctctctagtttttgaaactgtaatgacagaagacatttcagggttgttgtggggattttttccccccagttctaaaatggtcattgatagtgtcgatcttacctaactctcctcctatttaaaatttctgtggatctaccagataacctaaagtgggcatggattatttattatggctcctttaatgcccaaatagcaatcagtctaaaaccttaaaacaaaaacaaaaacaaaaacaaaaatcaaggagagaccagccacctcctgaaaaataggaataatagtatcagccactgacccatggataatgttgttgaaagcctgattaaagtatttttttctaaaaagaaaactcttcctgaattaagaaaataaaaatgggactttaaaaaaaaa

SEQ ID NO: 88 - DNA, 1015 bp, Homo sapiens
  source 1..1015: mol_type = genomic DNA
gtgctcccggaaaggcactgggcgaccgcacccgtggctttctctgggcgaccgggtcccagactccccccagcacagcagagcgcttccctgcccacccgcggaaaccgccccaggtgggccgcgccccctccccagcagccagcagggcgccagggctgagccggccgtggaggggagcgggtcccgcgggttatacaggcgccggggctccgcggcaggatgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagaccaaagcaaaggccaaagccaagaaagggaagggaaaggactagacgccaagcctggatgccaaggagcccctggtgtcacatggggcctggcccacgccctccctctcccaggcccgagatgtgacccaccagtgccttctgtctgctcgttagctttaatcaatcatgccctgccttgtccctctcactccccagccccacccctaagtgcccaaagtggggagggacaagggattctgggaagcttgagcctcccccaaagcaatgtgagtcccagagcccgcttttgttcttccccacaattccattactaagaaacacatcaaataaactgactttttccccccaataaaagctcttcttttttaatatataaaagccccttaaaaaaaaaaaaaaaaaa

SEQ ID NO: 89 - DNA, 2853 bp, Homo sapiens
  source 1..2853: mol_type = genomic DNA
agagacagaaggtggatagacaaatctccaccttcagactggtaggctcctccagaagccatcagacaggaagatgtgaaaatccccagcactcatcccagaatcactaagtggcacctgtcctgggccaaagtcccaggacagacctcattgttcctctgtgggaatacctccccaggagggcatcctggatttcccccttgcaacccaggtcagaagtttcatcgtcaaggttgtttcatcttttttttcctgtctaacagctctgactaccacccaaccttgaggcacagtgaagacatcggtggccactccaataacagcaggtcacagctgctcttctggaggtgtcctacaggtgaaaagcccagcgacccagtcaggatttaagtttacctcaaaaatggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaagacctcctgcctaagtgcagccccgtggggttcctcccttctcttcacagtcacattccaagcctcatgtccactggttcttcttggtctcagtgtcaatgcagcccccattgtggtcacaggaagtagaggaggccacgttcttactagtttcccttgcatggtttagaaagcttgccctggtgcctcaccccttgccataattactatgtcatttgctggagctctgcccatcctgcccctgagcccatggcactctatgttctaagaagtgaaaatctacactccagtgagacagctctgcatactcattaggatggctagtatcaaaagaaagaaaatcaggctggccaacggggtgaaaccctgtctctactaaaaatacaaaaaaaaaaaaaaattagccgggcgtggtggtgagtgcctgtaatcacagctacttgggaggctgagatgggagaatcacttgaacccgggaggcagaggttgcagtgagccgagattgtgcccctgcactccagcctgagcgacagtgagactctgtctcagtccatgaagatgtagaggagaaactggaactctcgagcgttgctgggggggattgtaaaatggtgtgaccactgcagaagacagtatggcagctttcctcaaaacttcagacatagaattaacacatgatcctgcaattccacttataggaattgacccacaagaaatgaaagcagggacttgaacccatatttgtacaccaatattcatagcagcttattcacaagacccaaaaggcagaagcaacccaaatgttcatcaatgaatgaatgaatggctaagcaaaatgtgatatgtacctaacgaagtatccttcagcctgaaagaggaatgaagtactcatacatgttacaacacggacgaaccttgaaaactttatgctaagtgaaataagccagacatcaacagataaatagtttatgattccacctacatgaggtactgagagtgaacaaatttacagagacagaaagcagaacagtgattaccagggactgaggggaggggagcatgggaagtgacggtttaatgggcacagggtttatgtttaggatgttgaaaaagttctgcagataaacagtagtgatagttgtaccgcaatgtgacttaatgccactaaattgacacttaaaaatggtttaaatggtcaattttgttatgtatattttatatcaatttaaaaaaaaacctgagccccaaaaggtattttaatcaccaaggctgattaaaccaaggctagaaccacctgcctatattttttgttaaatgatttcattcaatatcttttttttaataaaccatttttacttgggtgttta

SEQ ID NO: 90 - DNA, 6239 bp, Homo sapiens
  source 1..6239: mol_type = genomic DNA
gtagtctctttggaaacttctgcaggggaaaagagctaggaaagagctgcaaagcagtgtgggctttttcccttttttgctccttttcattacccctcctccgttttcacccttctccggacttcgcgtagaacctgcgaatttcgaagaggaggtggcaaagtgggagaaaagaggtgttagggtttggggtttttttgtttttgtttttgttttttaatttcttgatttcaacattttctcccaccctctcggctgcagccaacgcctcttacctgttctgcggcgccgcgcaccgctggcagctgagggttagaaagcggggtgtattttagattttaagcaaaaattttaaagataaatccatttttctctcccacccccaacgccatctccactgcatccgatctcattatttcggtggttgcttgggggtgaacaattttgtggctttttttcccctataattctgacccgctcaggcttgagggtttctccggcctccgctcactgcgtgcacctggcgctgccctgcttcccccaacctgttgcaaggctttaattcttgcaactgggacctgctcgcaggcaccccagccctccacctctctctacatttttgcaagtgtctgggggagggcacctgctctacctgccagaaattttaaaacaaaaacaaaaacaaaaaaatctccgggggccctcttggcccctttatccctgcactctcgctctcctgccccaccccgaggtaaagggggcgactaagagaagatggtgttgctcaccgcggtcctcctgctgctggccgcctatgcggggccggcccagagcctgggctccttcgtgcactgcgagccctgcgacgagaaagccctctccatgtgcccccccagccccctgggctgcgagctggtcaaggagccgggctgcggctgctgcatgacctgcgccctggccgaggggcagtcgtgcggcgtctacaccgagcgctgcgcccaggggctgcgctgcctcccccggcaggacgaggagaagccgctgcacgccctgctgcacggccgcggggtttgcctcaacgaaaagagctaccgcgagcaagtcaagatcgagagagactcccgtgagcacgaggagcccaccacctctgagatggccgaggagacctactcccccaagatcttccggcccaaacacacccgcatctccgagctgaaggctgaagcagtgaagaaggaccgcagaaagaagctgacccagtccaagtttgtcgggggagccgagaacactgcccacccccggatcatctctgcacctgagatgagacaggagtctgagcagggcccctgccgcagacacatggaggcttccctgcaggagctcaaagccagcccacgcatggtgccccgtgctgtgtacctgcccaattgtgaccgcaaaggattctacaagagaaagcagtgcaaaccttcccgtggccgcaaacgtggcatctgctggtgcgtggacaagtacgggatgaagctgccaggcatggagtacgttgacggggactttcagtgccacaccttcgacagcagcaacgttgagtgatgcgtccccccccaacctttccctcaccccctcccacccccagccccgactccagccagcgcctccctccaccccaggacgccactcatttcatctcatttaagggaaaaatatatatctatctatttgaggaaactgaggacctcggaatctctagcaagggctcaacttcgaaaatggcaacaacagagatgcaaaaagctaaaaagacaccccccccctttaaatggttttctttttgaggcaagttggatgaacagagaagggaagagaggaagaacgagaggaagagaagggaaggaagtgtttgtgtagaagagagagaaagacgaatagagttaggaaaaggaagacaagcaggtgggcaggaaggacatgcaccgagaccaggcaggggcccaactttcacgtccagccctggcctggggtcgggagaggtgggcgctagaagatgcagcccaggatgtggcaatcaatgacactattggggtttcccaggatggattggtcagggggagaaaggaaaaggcaaaacactccaggacctctcccggatctgtctcctcctctagccagcagtatggacagctggacccctgaacttcctctcctcttacctgggcagagtgttgtctctccccaaatttataaaaactaaaatgcattccattcctctgaaagcaaaacaaattcataattgagtgatattaaatagagaggttttcggaagcagatctgtgaatatgaaatacatgtgcatatttcattccccaggcagacattttttagaaatcaatacatgccccaatattggaaagacttgttcttccacggtgactacagtacatgctgaagcgtgccgtttcagccctcatttaattcaatttgtaagtagcgcagcagcctctgtgggggaggataggctgaaaaaaaaaagtgggctcgtatttatctacaggactccatatagtcatatataggcatataaatctattctttttctttgtttttttctttcttcctttctttcaaaggtttgcattaacttttcaaagtagttcctataggggcattgaggagcttcctcattctgggaaaactgagaaaacccatattctcctaatacaacccgtaatagcatttttgcctgcctcgaggcagagtttcccgtgagcaataaactcagcttttttgtggggcacagtactggatttgacagtgattccccacgtgtgttcatctgcacccaccgagccaggcagaggccagccctccgtggtgcacacagcacgcgcctcagtccatcccattttagtctttaaaccctcaggaagtcacagtctccggacaccacaccacatgagcccaacaggtccacgatggatccaccagtcccaccccagccttttcctttcatctgaacagaatgtgcatttttggaagcctccctcactctccatgctggcagagcaggagggagactgaagtaagagatggcagagggagatggtggcaaaaaggtttagatgcaggagaacagtaagatggatggttccggccagagtcgatgtggggaggaacagagggctgaagggagagggggctgactgttccattctagctttggcacaaagcagcagaaagggggaaaagccaatagaaatttccttagcttccccaccatatgtattttctaggatttgagaggaaagagaggaaaatgggggaatgggttgcaaaatagaaatgagcttaatccaggccgcagagccagggaaggtgagtaactttaggagggtgctagactttagaagccagataggaagaatcagtctaaactggccatgctttggaagggacaagactatgtgctccgctgcccaccttcagcctgcaatgagggactgaggcccacgagtctttccagctcttcctccattctggccagtccctgcatcctccctggggtggaggatggaaggaaagctgggacaagcagggaacgcatgattcagggatgctgtcactcggcagccagattccgaaactcccattctccaatgacttcctcaaccaatgggtggccttgtgactgttctttaaggctgaagatatccaggaaagggggcttggacactggccaaggagaccccttcgtgctgtggacacagctctcttcactctttgctcatggcatgacacagcggagaccgcctccaacaacgaatttggggctacgaagaggaatagcgaaaaagcaaatctgtttcaactgatgggaaccctatagctatagaacttgggggctatctcctatgcccctggacaggacagttggctggggacaggagaagtgctcaatcttcatgagacaaaggggcccgatagggccagcagccacaaggccttgacctgccgagtcagcatgccccatctctctgcacagctgtcccctaaacccaactcacgtttctgtatgtcttaggccagtatcccaaacctcttccacgtcactgttctttccacccattctccctttgcatcttgagcagttatccaactaggatctgccaagtggatactggggtgccactcccctgagaaaagactgagccaggaactacaagctccccccacattcctcccagcctggacctaattcttgagaggggctctctcttcacggactgtgtctggactttgagcaggcttctgccccttgcgttggctctttgctgccagccatcaggtgggggattagagcctggtgtaagtgcgccagactcttccggtttccaaagttcgtgcctgcgaacccaaacctgtgagtctcttctgcatgcaggagtttctcctgggcagctggtcactccccagagaagctgggccttcatggacacatggaactaagcctcccaaatgggagttctggctgagcccagggtggggagatcctgggaagggaggcactggaggaagacggcacctcttcccccatggcagggtgtgagggaggcaggtttggaatggtgcgagtatggcaatctaagcaggggtctggtctctttgactccaggctggcctttggccgactgtctgctcacccagagaccttggactccggactatccatggctccgaatctaagtgctgcccactcccatgctcacacccacagaaggtcttcccatcccctttagattcgtgcctcactccaccagtgaggaagatgcctctgtctttcccacgactgccaggagatagggaagcccagccaggactgaccctccttcctccagcctgccctgacccacctggcaaagcagggcacatggggaggaagagactggaacctttctttgacagccaggcctagacagacaggcctggggacactggccccatgaggggaggaaggcaggcgcacgaggtccagggaggcccttttctgatcatgccccttctctcccaccccatctccccaccaccacctctgtggcctccatggtacccccacagggctggcctcccctagagggtgggcctcaaccacctgctcccgccacgcaccggttagtgagacagggctgccacggcaaccgccaagcccccctcaaggtgggacagtaccccggacccatccactcactcctgagagggctccggcccagaatgggaacctcagagaagagctctaaggagaagaaaccccatagcgtcagagaggatatgtctggcttccaagagaaaggaggctccgttttgcaaagtggaggagggacgagggacaggggtttcaccagccagcaacctgggccttgtactgtctgtgtttttaaaaccactaaagtgcaagaattacattgcactgtttctccactttttattttctcttaggcttttgtttctatttcaaacatactttcttggttttctaatggagtatatagtttagtcatttcacagactctggcctcctctcctgaaatccttttggatggggaaagggaaggtggggagggtccgaggggaaggggaccccagcttccctgtgcccgctcaccccactccaccagtccccggtcgccagccggagtctcctctctaccgccactgtcacaccgtagcccacatggatagcacagttgtcagacaagattccttcagattccgagttgcctaccggttgttttcgttgttgttgttgttgtttttctttttctttttttttttgaagacagcaataaccacagtacatattactgtagttctctatagttttacatacattcataccataactctgttctctcctcttttttgttttcaactttaaaaacaaaaataaacgatgataatctttactggtgaaaaggatggaaaaataaatcaacaaatgcaaccagtttgtgagaaaaaaaaaaaaaagccgaaaaaaaaaaaaaaaacacctgaatgcggaagagctcggctcccgtttagcattttgtacttaaggaaataaaaaaccaacaaaggatctcacattttcttaaaaagtgaagattgctgtatactatttattcaacttataatttatgttactccttgatctttgtcttttgtcatgacaaagcatttatttaataaagttatgcattcagtta

SEQ ID NO: 91 - DNA, 6927 bp, Homo sapiens
  source 1..6927: mol_type = genomic DNA
gcattaatagtaatacagataacgggtttgaaagaattctctactgaagaaggattgaatttttccagggtgctgatacagagaagaaacccgacttcactctctccctatttccccactcttaggtttaaaagtctgtcacctttcgcttggtttaaactcggaaaggtctcagtgcacagcaaagttgcagggctgcgtctgcactacggagcctctagattgctgaaaacagtcttatggaaggataacacattgtctgtcactggctggttgtaatgcaaggaagggacaaagatgaaatggaaacatgttccttttttggtcatgatatcactcctcagcttatccccaaatcacctgtttctggcccagcttattccagaccctgaagatgtagagagggggaacgaccacgggacgccaatccccacctctgataacgatgacaattcgctgggctatacaggctcccgtcttcgtcaggaagattttccacctcgcattgttgaacacccttcagacctgattgtctcaaaaggagaacctgcaactttgaactgcaaagctgaaggccgccccacacccactattgaatggtacaaagggggagagagagtggagacagacaaagatgaccctcgctcacaccgaatgttgctgccgagtggatctttatttttcttacgtatagtacatggacggaaaagtagacctgatgaaggagtctatgtctgtgtagcaaggaattaccttggagaggctgtgagccacaatgcatcgctggaagtagccatacttcgggatgacttcagacaaaacccttcggatgtcatggttgcagtaggagagcctgcagtaatggaatgccaacctccacgaggccatcctgagcccaccatttcatggaagaaagatggctctccactggatgataaagatgaaagaataactatacgaggaggaaagctcatgatcacttacacccgtaaaagtgacgctggcaaatatgtttgtgttggtaccaatatggttggggaacgtgagagtgaagtagccgagctgactgtcttagagagaccatcatttgtgaagagacccagtaacttggcagtaactgtggatgacagtgcagaatttaaatgtgaggcccgaggtgaccctgtacctacagtacgatggaggaaagatgatggagagctgcccaaatccagatatgaaatccgagatgatcataccttgaaaattaggaaggtgacagctggtgacatgggttcatacacttgtgttgcagaaaatatggtgggcaaagctgaagcatctgctactctgactgttcaagaacctccacattttgttgtgaaaccccgtgaccaggttgttgctttgggacggactgtaacttttcagtgtgaagcaaccggaaatcctcaaccagctattttctggaggagagaagggagtcagaatctacttttctcatatcaaccaccacagtcatccagccgattttcagtctcccagactggcgacctcacaattactaatgtccagcgatctgatgttggttattacatctgccagactttaaatgttgctggaagcatcatcacaaaggcatatttggaagttacagatgtgattgcagatcggcctcccccagttattcgacaaggtcctgtgaatcagactgtagccgtggatggcactttcgtcctcagctgtgtggccacaggcagtccagtgcccaccattctgtggagaaaggatggagtcctcgtttcaacccaagactctcgaatcaaacagttggagaatggagtactgcagatccgatatgctaagctgggtgatactggtcggtacacctgcattgcatcaacccccagtggtgaagcaacatggagtgcttacattgaagttcaagaatttggagttccagttcagcctccaagacctactgacccaaatttaatccctagtgccccatcaaaacctgaagtgacagatgtcagcagaaatacagtcacattatcgtggcaaccaaatttgaattcaggagcaactccaacatcttatattatagaagccttcagccatgcatctggtagcagctggcagaccgtagcagagaatgtgaaaacagaaacatctgccattaaaggactcaaacctaatgcaatttaccttttccttgtgagggcagctaatgcatatggaattagtgatccaagccaaatatcagatccagtgaaaacacaagatgtcctaccaacaagtcagggggtggaccacaagcaggtccagagagagctgggaaatgctgttctgcacctccacaaccccaccgtcctttcttcctcttccatcgaagtgcactggacagtagatcaacagtctcagtatatacaaggatataaaattctctatcggccatctggagccaaccacggagaatcagactggttagtttttgaagtgaggacgccagccaaaaacagtgtggtaatccctgatctcagaaagggagtcaactatgaaattaaggctcgccctttttttaatgaatttcaaggagcagatagtgaaatcaagtttgccaaaaccctggaagaagcacccagtgccccaccccaaggtgtaactgtatccaagaatgatggaaacggaactgcaattctagttagttggcagccacctccagaagacactcaaaatggaatggtccaagagtataaggtttggtgtctgggcaatgaaactcgataccacatcaacaaaacagtggatggttccaccttttccgtggtcattccctttcttgttcctggaatccgatacagtgtggaagtggcagccagcactggggctgggtctggggtaaagagtgagcctcagttcatccagctggatgcccatggaaaccctgtgtcacctgaggaccaagtcagcctcgctcagcagatttcagatgtggtgaagcagccggccttcatagcaggtattggagcagcctgttggatcatcctcatggtcttcagcatctggctttatcgacaccgcaagaagagaaacggacttactagtacctacgcgggtatcagaaaagtcccgtcttttaccttcacaccaacagtaacttaccagagaggaggcgaagctgtcagcagtggagggaggcctggacttctcaacatcagtgaacctgccgcgcagccatggctggcagacacgtggcctaatactggcaacaaccacaatgactgctccatcagctgctgcacggcaggcaatggaaacagcgacagcaacctcactacctacagtcgcccagctgattgtatagcaaattataacaaccaactggataacaaacaaacaaatctgatgctccctgagtcaactgtttatggtgatgtggaccttagtaacaaaatcaatgagatgaaaaccttcaatagcccaaatctgaaggatgggcgttttgtcaatccatcagggcagcctactccttacgccaccactcagctcatccagtcaaacctcagcaacaacatgaacaatggcagcggggactctggcgagaagcactggaaaccactgggacagcagaaacaagaagtggcaccagttcagtacaacatcgtggagcaaaacaagctgaacaaagattatcgagcaaatgacacagttcctccaactatcccatacaaccaatcatacgaccagaacacaggaggatcctacaacagctcagaccggggcagtagtacatctgggagtcaggggcacaagaaaggggcaagaacacccaaggtaccaaaacagggtggcatgaactgggcagacctgcttcctcctcccccagcacatcctcctccacacagcaatagcgaagagtacaacatttctgtagatgaaagctatgaccaagaaatgccatgtcccgtgccaccagcaaggatgtatttgcaacaagatgaattagaagaggaggaagatgaacgaggccccactccccctgttcggggagcagcttcttctccagctgccgtgtcctatagccatcagtccactgccactctgactccctccccacaggaagaactccagcccatgttacaggattgtccagaggagactggccacatgcagcaccagcccgacaggagacggcagcctgtgagtcctcctccaccaccacggccgatctcccctccacatacctatggctacatttcaggacccctggtctcagatatggatacggatgcgccagaagaggaagaagacgaagccgacatggaggtagccaagatgcaaaccagaaggcttttgttacgtgggcttgagcagacacctgcctccagtgttggggacctggagagctctgtcacggggtccatgatcaacggctggggctcagcctcagaggaggacaacatttccagcggacgctccagtgttagttcttcggacggctcctttttcactgatgctgactttgcccaggcagtcgcagcagcggcagagtatgctggtctgaaagtagcacgacggcaaatgcaggatgctgctggccgtcgacattttcatgcgtctcagtgccctaggcccacaagtcccgtgtctacagacagcaacatgagtgccgccgtaatgcagaaaaccagaccagccaagaaactgaaacaccagccaggacatctgcgcagagaaacctacacagatgatcttccaccacctcctgtgccgccacctgctataaagtcacctactgcccaatccaagacacagctggaagtacgacctgtagtggtgccaaaactcccttctatggatgcaagaacagacagatcatcagacagaaaaggaagcagttacaaggggagagaagtgttggatggaagacaggttgttgacatgcgaacaaatccaggtgatcccagagaagcacaggaacagcaaaatgacgggaaaggacgtggaaacaaggcagcaaaacgagaccttccaccagcaaagactcatctcatccaagaggatattctaccttattgtagacctacttttccaacatcaaataatcccagagatcccagttcctcaagctcaatgtcatcaagaggatcaggaagcagacaaagagaacaagcaaatgtaggtcgaagaaatattgcagaaatgcaggtacttggaggatatgaaagaggagaagataataatgaagaattagaggaaactgaaagctgaagacaaccaagaggcttatgagatctaatgtgaaaatcatcactcaagatgcctcctgtcagatgacacatgacgccagataaaatgttcagtgcaatcagagtgtacaaattgtcgtttttattcctcttattgggatatcattttaaaaactttattgggtttttattgttgttgtttgatccctaaccctacaaagagccttcctattcccctcgctgttggagcaaaccattataccttacttccagcaagcaaagtgctttgacttcttgcttcagtcatcagccagcaagagggaacaaaactgttcttttgcattttgccgctgagatatggcattgcactgcttatatgccaagctaatttatagcaagatattgatcaaatatagaaagttgatattcaacctcacaagggctctcaaagtataatctttctatagccaactgctaatgcaaattaaaacatatttcattttaacatgatttcaaaatcagtttttcatactaccctttgctggaagaaactaaaaatatagcaaatgcagaaccacaaacaattcgaatggggtagaaacattgtaaatatttactctttgcaaaccctggtggtattttattttggcttcatttcaatcattgaagtatattcttattggaaatgtacttttggataagtagggctaagccagttggatctctggttgtctagtcattgtcataagtaaacctagtaaaaccttgttctatttttcaatcatcaaaaagtaattataaatacgtattacaaacaagtggatgtttttaatgaccaattgagtaagaacatccctgtcttaactggcctaaatttcttctggtagtgtcagttcaactttcagaagtgccacttaaggaagtttgatttttgtttttgtaatgcactgtttttaatctctctctctttttttttttttttttggttttaaaagcacaatcactaaactttatttgtaaaccattgtaactattaaccttttttgtcttattgaaaaaaaaaatgttgagaagcgtttttaacctgttttgttaatgctctatgtttgtatttggaatatttgaataatgacagatggtgaagtaacatgcatactttattgtgggccatgaaccaaatggttcttacttttcctggacttaaagaaaaaaagaggtttaagtttgttgtggccaatgtcgaaacctacaagatttccttaaaatctctaatagaggcattacttgctttcaattgacaaatgatgccctctgactagtagatttctatgatccttttttgtcattttatgaatatcattgattttataattggtgctatttgaagaaaaaaatgtacatttattcatagatagataagtatcaggtctgaccccagtggaaaacaaagccaaacaaaactgaaccacaaaaaaaaaggctggtgttcaccaaaaccaaacttgttcatttagataatttgaaaaagttccatagaaaaggcgtgcagtactaagggaacaatccatgtgattaatgttttcattatgttcatgtaagaagccccttatttttagccataattttgcatactgaaaatccaataatcagaaaagtaattttgtcacattatttattaaaaatgttctcaaataca

SEQ ID NO: 92 - DNA, 5841 bp, Homo sapiens
  source 1..5841: mol_type = genomic DNA
aacagttgcctgcgcgccctcgccggaccggcggctccctagttgcgccccgaccaggccctgcccttgctgccggctcgcgcgcgtccgcgccccctccattcctgggcgcatcccagctctgccccaactcgggagtccaggcccgggcgccagtgcccgcttcagctccggttcactgcgcccgccggacgcgcgccggaggactccgcagccctgctcctgaccgtccccccaggcttaacccggtcgctccgctcggattcctcggctgcgctcgctcgggtggcgacttcctccccgcgccccctccccctcgccatgaagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgaaggtggaactgcagcactgtggataacacctctgtttttggcagggtgatgcagataggcagccgcgagacggccttcacatacgcggtgagcgcagcaggggtggtgaacgccatgagccgggcgtgccgcgagggcgagctgtccacctgcggctgcagccgcgccgcgcgccccaaggacctgccgcgggactggctctggggcggctgcggcgacaacatcgactatggctaccgctttgccaaggagttcgtggacgcccgcgagcgggagcgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtggctacgaccagttcaagaccgtgcagacggagcgctgccactgcaagttccactggtgctgctacgtcaagtgcaagaagtgcacggagatcgtggaccagtttgtgtgcaagtagtgggtgccacccagcactcagccccgctcccaggacccgcttatttatagaaagtacagtgattctggtttttggtttttagaaatattttttatttttccccaagaattgcaaccggaaccattttttttcctgttaccatctaagaactctgtggtttattattaatattataattattatttggcaataatgggggtgggaaccaagaaaaatatttattttgtggatctttgaaaaggtaatacaagacttcttttgatagtatagaatgaaggggaaataacacataccctaacttagctgtgtggacatggtacacatccagaaggtaaagaaatacattttctttttctcaaatatgccatcatatgggatgggtaggttccagttgaaagagggtggtagaaatctattcacaattcagcttctatgaccaaaatgagttgtaaattctctggtgcaagataaaaggtcttgggaaaacaaaacaaaacaaaacaaacctcccttccccagcagggctgctagcttgctttctgcattttcaaaatgataatttacaatggaaggacaagaatgtcatattctcaaggaaaaaaggtatatcacatgtctcattctcctcaaatattccatttgcagacagaccgtcatattctaatagctcatgaaatttgggcagcagggaggaaagtccccagaaattaaaaaatttaaaactcttatgtcaagatgttgatttgaagctgttataagaattaggattccagattgtaaaaagatccccaaatgattctggacactagatttttttgtttggggaggttggcttgaacataaatgaaaatatcctgttattttcttagggatacttggttagtaaattataatagtaaaaataatacatgaatcccattcacaggttctcagcccaagcaacaaggtaattgcgtgccattcagcactgcaccagagcagacaacctatttgaggaaaaacagtgaaatccaccttcctcttcacactgagccctctctgattcctccgtgttgtgatgtgatgctggccacgtttccaaacggcagctccactgggtcccctttggttgtaggacaggaaatgaaacattaggagctctgcttggaaaacagttcactacttagggatttttgtttcctaaaacttttattttgaggagcagtagttttctatgttttaatgacagaacttggctaatggaattcacagaggtgttgcagcgtatcactgttatgatcctgtgtttagattatccactcatgcttctcctattgtactgcaggtgtaccttaaaactgttcccagtgtacttgaacagttgcatttataaggggggaaatgtggtttaatggtgcctgatatctcaaagtcttttgtacataacatatatatatatatacatatatataaatataaatataaatatatctcattgcagccagtgatttagatttacagtttactctggggttatttctctgtctagagcattgttgtccttcactgcagtccagttgggattattccaaaagttttttgagtcttgagcttgggctgtggccctgctgtgatcataccttgagcacgacgaagcaaccttgtttctgaggaagcttgagttctgactcactgaaatgcgtgttgggttgaagatatcttttttcttttctgcctcacccctttgtctccaacctccatttctgttcactttgtggagagggcattacttgttcgttatagacatggacgttaagagatattcaaaactcagaagcatcagcaatgtttctcttttcttagttcattctgcagaatggaaacccatgcctattagaaatgacagtacttattaattgagtccctaaggaatattcagcccactacatagatagcttttttttttttttttttaataaggacacctctttccaaacagtgccatcaaatatgttcttatctcagacttacgttgttttaaaagtttggaaagatacacatctttcataccccccttaggcaggttggctttcatatcacctcagccaactgtggctcttaatttattgcataatgatattcacatcccctcagttgcagtgaattgtgagcaaaagatcttgaaagcaaaaagcactaattagtttaaaatgtcacttttttggtttttattatacaaaaaccatgaagtactttttttatttgctaaatcagattgttcctttttagtgactcatgtttatgaagagagttgagtttaacaatcctagcttttaaaagaaactatttaatgtaaaatattctacatgtcattcagatattatgtatatcttctagcctttattctgtacttttaatgtacatatttctgtcttgcgtgatttgtatatttcactggtttaaaaaacaaacatcgaaaggcttatgccaaatggaagatagaatataaaataaaacgttacttgtatattggtaagtggtttcaattgtccttcagataattcatgtggagatttttggagaaaccatgacggatagtttaggatgactacatgtcaaagtaataaaagagtggtgaattttaccaaaaccaagctatttggaagcttcaaaaggtttctatatgtaatggaacaaaaggggaattctcttttcctatatatgttccttacaaaaaaaaaaaaaaaagaaatcaagcagatggcttaaagctggttataggattgctcacattcttttagcattatgcatgtaacttaattgttttagagcgtgttgctgttgtaacatcccagagaagaatgaaaaggcacatgcttttatccgtgaccagatttttagtccaaaaaaatgtatttttttgtgtgtttaccactgcaactattgcacctctctatttgaatttactgtggaccatgtgtggtgtctctatgccctttgaaagcagtttttataaaaagaaagcccgggtctgcagagaatgaaaactggttggaaactaaaggttcattgtgttaagtgcaattaatacaagttattgtgcttttcaaaaatgtacacggaaatctggacagtgctccacagattgatacattagcctttgctttttctctttccggataaccttgtaacatattgaaaccttttaaggatgccaagaatgcattattccacaaaaaaacagcagaccaacatatagagtgtttaaaatagcatttctgggcaaattcaaactcttgtggttctaggactcacatctgtttcagtttttcctcagttgtatattgaccagtgttctttattgcaaaaacatatacccgatttagcagtgtcagcgtattttttcttctcatcctggagcgtattcaagatcttcccaatacaagaaaattaataaaaaatttatatataggcagcagcaaaagagccatgttcaaaatagtcattatgggctcaaatagaaagaagacttttaagttttaatccagtttatctgttgagttctgtgagctactgacctcctgagactggcactgtgtaagttttagttgcctaccctagctcttttctcgtacaattttgccaataccaagtttcaatttgtttttacaaaacattattcaagccactagaattatcaaatatgacgctatagcagagtaaatactctgaataagagaccggtactagctaactccaagagatcgttagcagcatcagtccacaaacacttagtggcccacaatatatagagagatagaaaaggtagttataacttgaagcatgtatttaatgcaaataggcacgaaggcacaggtctaaaatactacattgtcactgtaagctatacttttaaaatatttattttttttaaagtattttctagtcttttctctctctgtggaatggtgaaagagagatgccgtgttttgaaagtaagatgatgaaatgaatttttaattcaagaaacattcagaaacataggaattaaaacttagagaaatgatctaatttccctgttcacacaaactttacactttaatctgatgattggatattttattttagtgaaacatcatcttgttagctaactttaaaaaatggatgtagaatgattaaaggttggtatgatttttttttaatgtatcagtttgaacctagaatattgaattaaaatgctgtctcagtattttaaaagcaaaaaaggaatggaggaaaattgcatcttagaccatttttatatgcagtgtacaatttgctgggctagaaatgagataaagattatttatttttgttcatatcttgtacttttctattaaaatcattttatgaaatcca

SEQ ID NO: 93 - DNA, 6844 bp, Homo sapiens
  source 1..6844: mol_type = genomic DNA
gggctcgggcgccggccgggcttgcgcagcgctaggctcctcgccgctcctagtctgcggcggcggcggcggggagggcgcggccgcgggcggggctgagggcggcgggggcgggccgcccgagctgggagggcggcggcgccgaggggaggagagcggcccatggacccgcggggcccggcgccccagactctgcgccgtcgggacggagcccaagatgtcggcctaggccggggcgcgacgacgcggacggggcggcgaggaggcgccgctgctgccggggctcgcagccgccgagcccccgagggcgcgccctgacggactggccgagccggcggtgagaggccggcgcgtcgggagcgggccgcgcggcaccatgtcggccaaggtgcggctcaagaagctggagcagctgctcctggacgggccctggcgcaacgagagcgccctgagcgtggaaacgctgctcgacgtgctcgtctgcctgtacaccgagtgcagccactcggccctgcgccgcgacaagtacgtggccgagttcctcgagtgggctaaaccatttacacagctggtgaaagaaatgcagcttcatcgagaagactttgaaataattaaagtaattggaagaggtgcttttggtgaggttgctgttgtcaaaatgaagaatactgaacgaatttatgcaatgaaaatcctcaacaagtgggagatgctgaaaagagcagagaccgcgtgcttccgagaggagcgcgatgtgctggtgaacggcgactgccagtggatcaccgcgctgcactacgcctttcaggacgagaaccacctgtacttagtcatggattactatgtgggtggtgatttactgaccctgctcagcaaatttgaagacaagcttccggaagatatggcgaggttctacattggtgaaatggtgctggccattgactccatccatcagcttcattacgtgcacagagacattaaacctgacaatgtccttttggacgtgaatggtcatatccgcctggctgactttggatcatgtttgaagatgaatgatgatggcactgtgcagtcctccgtggccgtgggcacacctgactacatctcgccggagatcctgcaggcgatggaggacggcatgggcaaatacgggcctgagtgtgactggtggtctctgggtgtctgcatgtatgagatgctctatggagaaacgccgttttatgcggagtcactcgtggagacctatgggaagatcatgaaccatgaagagcgattccagttcccatcccatgtcacggatgtatctgaagaagcgaaggacctcatccagagactgatctgcagtagagaacgccggctggggcagaatggaatagaggatttcaaaaagcatgcgttttttgaaggtctaaattgggaaaatatacgaaacctagaagcaccttatattcctgatgtgagcagtccctctgacacatccaacttcgacgtggatgacgacgtgctgagaaacacggaaatattacctcctggttctcacacaggcttttctggattacatttgccattcattggttttacattcacaacggaaagctgtttttctgatcgaggctctctgaagagcataatgcagtccaacacattaaccaaagatgaggatgtgcagcgggacctggagcacagcctgcagatggaagcttacgagaggaggattcggaggctggaacaggagaagctggagctgagcaggaagctgcaagagtccacccagaccgtgcagtccctccacggctcatctcgggccctcagcaattcaaaccgagataaagaaatcaaaaagctaaatgaagaaatcgaacgcttgaagaataaaatagcagattcaaacaggctcgagcgacagcttgaggacacagtggcgcttcgccaagagcgtgaggactccacgcagcggctgcgggggctggagaagcagcaccgcgtggtccggcaggagaaggaggagctgcacaagcaactggttgaagcctcagagcggttgaaatcccaggccaaggaactcaaagatgcccatcagcagcgaaagctggccctgcaggagttctcggagctgaacgagcgcatggcagagctccgtgcccagaagcagaaggtgtcccggcagctgcgagacaaggaggaggagatggaggtggccacgcagaaggtggacgccatgcggcaggaaatgcggagagctgagaagctcaggaaagagctggaagctcagcttgatgatgctgttgctgaggcctccaaggagcgcaagcttcgtgagcacagcgagaacttctgcaagcaaatggaaagcgagctggaggccctcaaggtgaagcaaggaggccggggagcgggtgccaccttagagcaccagcaagagatttccaaaatcaaatccgagctggagaagaaagtcttattttatgaagaggaattggtcagacgtgaggcctcccatgtgctagaagtgaaaaatgtgaagaaggaggtgcatgattcagaaagccaccagctggccctgcagaaagaaatcttgatgttaaaagataagttagaaaagtcaaagcgagaacggcataacgagatggaggaggcagtaggtacaataaaagataaatacgaacgagaaagagcgatgctgtttgatgaaaacaagaagctaactgctgaaaatgaaaagctctgttcctttgtggataaactcacagctcaaaatagacagctggaggatgagctgcaggatctggcagccaagaaggagtcagtggcccactgggaagctcagattgcggaaatcattcagtgggtcagtgacgagaaagatgcccggggttaccttcaagctcttgcttccaagatgaccgaagagctcgaggctttgaggagttctagtctggggtcaagaacactggacccgctgtggaaggtgcgccgcagccagaagctggacatgtccgcgcggctggagctgcagtcggccctggaggcggagatccgggccaagcagcttgtccaggaggagctcaggaaggtcaaggacgccaacctcaccttggaaagcaaactaaaggattccgaagccaaaaacagagaattattagaagaaatggaaattttgaagaaaaagatggaagaaaaattcagagcagatactgggctcaaacttccagattttcaggattccatttttgagtatttcaacactgctcctcttgcacatgacctgacatttagaaccagctcagctagtgagcaagaaacacaagctccgaagccagaagcgtccccgtcgatgtctgtggctgcatcagagcagcaggaggacatggctcggcccccgcagaggccatccgctgtgccgttgcccaccacgcaggccctggctctggctggaccgaagccaaaagctcaccagttcagcatcaagtccttctccagccctactcagtgcagccactgcacctccctgatggttgggctgatccggcagggctacgcctgcgaggtgtgttcctttgcttgccacgtgtcctgcaaagacggtgccccccaggtgtgcccaatacctcccgagcagtccaagaggcctctgggcgtggacgtgcagcgaggcatcggaacagcctacaaaggccatgtcaaggtcccaaagcccacgggggtgaagaagggatggcagcgcgcatatgcagtcgtctgtgactgcaagctcttcctgtatgatctgcctgaaggaaaatccacccagcctggtgtcattgcgagccaagtcttggatctcagagatgacgagttttccgtgagctcagtcctggcctcagatgtcattcatgctacacgccgagatattccatgtatattcagggtgacggcctctctcttaggtgcaccttctaagaccagctcgctgctcattctgacagaaaatgagaatgaaaagaggaagtgggttgggattctagaaggactccagtccatccttcataaaaaccggctgaggaatcaggtcgtgcatgttcccttggaagcctacgacagctcgctgcctctcatcaaggccatcctgacagctgccatcgtggatgcagacaggattgcagtcggcctagaagaagggctctatgtcatagaggtcacccgagatgtgatcgtccgtgccgctgactgtaagaaggtacaccagatcgagcttgctcccagggagaagatcgtaatcctcctctgtggccggaaccaccatgtgcacctctatccgtggtcgtcccttgatggagcggaaggcagctttgacatcaagcttccggaaaccaaaggctgccagctcatggccacggccacactcaagaggaactctggcacctgcctgtttgtggccgtgaaacggctgatcctttgctatgagatccagagaacgaagccattccacagaaagttcaatgagattgtggctcccggcagcgtgcagtgcctggcggtgctcagggacaggctctgtgtgggctacccttctgggttctgcctgctgagcatccagggggacgggcagcctctaaacctggtaaatcccaatgacccctcgcttgcgttcctctcacaacagtcttttgatgccctttgtgctgtggagctcgaaagcgaggagtacctgctttgcttcagccacatgggactgtacgtggacccgcaaggccggagggcacgcgcgcaggagctcatgtggcctgcggctcctgtcgcctgtagttgcagccccacccacgtcacggtgtacagcgagtatggcgtggacgtctttgatgtgcgcaccatggagtgggtgcagaccatcggcctgcggaggataaggcccctgaactctgaaggcaccctcaacctcctcaactgcgagcctccacgcttgatctacttcaagagcaagttctcgggagcggttctcaacgtgccggacacctccgacaacagcaagaagcagatgctgcgcaccaggagcaaaaggcggttcgtcttcaaggtcccagaggaagagagactgcagcagaggcgagagatgcttagagacccagaattgagatccaaaatgatatccaacccaaccaacttcaaccacgtggcccacatgggcccaggcgacggcatgcaggtgctcatggacctgcctctgagtgctgtgcccccctcccaggaggaaaggccgggccccgctcccaccaacctggctcgccagcctccatccaggaacaagccctacatctcgtggccctcatcaggtggatcggagcctagcgtgactgtgcctctgagaagtatgtctgatccagaccaggactttgacaaagagcctgattcggactccaccaaacactcaactccatcgaatagctccaaccccagcggcccaccgagccccaactccccccacaggagccagctccccctcgaaggcctggagcagccggcctgtgacacctgaagccgccagctcgccacaggggccagggagctggagatggcctccagcgtcagtgccaagactgagcgggccctccagtgttgtccaaggaaatgtagaatcactttgtagatatggagatgaagaagacaaatctttattataatattgatcagttttatgccgcattgttcgtggcagtagaccacatctgttcgtctgcacagctgtgaggcgatgctgttccatctgcacatgaaggacccccatacagcctgtctcccacccctgacaacccgagagggcatatggggccctgccaacaccacttcctcagcagaaacccgtcatgacgcggctgcttcggaagcagacatctggggacacagcctcagtacccagtcttttccctagttcctgaaactttcctaggaccttaagagaatagtaggaggtcctatagcattcccagtgtcactagaattttgaagacaggaaagtggaggttagtctgtggcctttttttcatttagccattgcacagtcagctgcagaagtcctgctgaccacctagtcatggacaaaggcccaggaccagtgacaccctgcgtccctgtgtgcattaagttcattctgggtcgcagccatgaagtgtcaccagtatctactactgtgaagtcagctgtgctgttttccattcgcttccacggcttctgcctcctgccataaaaccagcgagtgtcgtggtgcaggcaggccctgtggcctgctgggctgagggaagtcagagccccagggcgccacgaagcagccactgggataccccaccccgccccgccctgcccgcccccccccccaccagtcctgcccccgcatggagcccccgtgattagtagcccgtatgatcacgtagacccacccaacacactcctgcacactggccccggcccacggcacagcaatcccctgcgcgtggatttcacctcaccctttgtaccagatgttgagtgaccagctctgtggccctgtgtcgtcagaggcttgtgattaactgtggcggcagacacagcttgtccacagcttgggccaggcttcccctgtcctcccaccggtcggctgcttggcaaggctgttcaggacgtgcacttccccaagtcggcactgagtggcccagcaccgcctagccctgccaccccactgccctcctgggccttctgctggatgggcacctggggggttctggtttttacttttttaatgtaagtctcagtctttgtaattaattattgaattgtgagaacatttttgaacaatttacctgtcaataaagcagaagacggcagttttaaagtt

SEQ ID NO: 94 - DNA, 3330 bp, Homo sapiens
  source 1..3330: mol_type = genomic DNA
aggggcgcgagcgggctggccgaggaggaggcggcggcgtggagctgcctcctgccggcgggccgggccgggccgagccccgggcgctgcggcgacgcctggatcctgcctccgccaggccggctgcctggtgccccgaggaggctgctgagccccaggccatggtcccctctcgcaggacgtggaacctgggagccacgccctcgctgcggggcctgtggagagtgggccgggccccggagcccgagccggggatggctcgccccgccccagccccagccagcccggccgcccgccctttcccacacaccggcccggggaggttgagaactgggcgtggaaaagataccccagtctgcggtgacgaggactccagtgcccgaagtgcagctcgcccagccctagctcagtgccgagcccttagcgtggactgggctggccccggaagcccccacgggctctacctgaccctgcaggtagaacacctgaaggagaagctcattagccaggcccaggaagtgagccgactgcgatctgagctggggggcaccgacttggagaagcaccgggacctgctgatggtggagaatgagcgactgaggcaggagatgcggcgctgtgaggccgagctgcaagagctgcgcacaaagccagcaggtccctgcccaggttgtgagcacagccaggagagcgcccagctccgtgacaagctgtcccagctgcagctggagatggcggaaagcaaaggcatgctgtcagagctgaacctagaggtgcagcagaagaccgaccggctggctgaggtggagctgcgactcaaggactgcctggctgagaaggcacaggaggaggagcggcttagtcggcgcctgcgtgacagccacgagaccattgccagcctgcgggcccagtccccacctgtcaagtatgtcatcaagacagtggaggtggagtcgtccaagaccaagcaggccctcagcgagtcccaggcccggaaccagcacctgcaggagcaggtggctatgcagaggcaggtgctgaaggagatggaacagcagctgcagagctcacaccagctgaccgcgcggctccgggcgcagattgccatgtacgagtcagagctggagcgggcccatgggcagatgctggaggagatgcagtccctggaagaggacaagaaccgggccattgaggaggcctttgccagagcccaggtggagatgaaggctgtgcacgagaatctagcaggcgtccggaccaacttgctgaccttgcagccggcactgcggaccctcaccaacgactacaatgggctcaagcggcaggtgcgcggcttcccactgctgctgcaggaggccctcaggagtgtcaaggccgagataggccaggccatcgaggaggtcaacagcaacaaccaggagctgctgcgcaagtaccgccgcgagctgcagctgcgtaagaagtgccacaatgagctcgtgcggctgaaagggaacatccgagtgattgctcgtgtccggccagtcaccaaagaggatggggaaggacctgaggccaccaatgctgtgactttcgatgccgacgacgactccatcatccacctgctgcacaagggcaagcctgtgtccttcgagctggacaaggtcttctccccacaggcctcgcagcaggacgtgttccaggaggtgcaggccctggtcacctcttgcattgatggcttcaatgtctgcatctttgcgtacggccagacgggcgccggcaagacgtacacgatggaggggaccgctgagaacccaggtatcaaccagcgggccctgcagctgctcttctccgaggtgcaggagaaggcgtctgactgggagtacaccatcaccgtcagcgctgcggagatctacaatgaggtcctcagggacctgctagggaaagagcctcaggaaaaactggagatccggctgtgcccagacggcagtgggcagctgtatgtaccagggctgactgagttccaagtgcagagcgtggacgacatcaacaaggtgtttgagtttggccacactaatcgcacgaccgagttcaccaacctgaacgagcacagctcccgctcgcacgcgctgctcatcgtgacggtgcgaggcgtggactgcagcacaggcctccgcaccacggggaagctgaacctggtggacttggctggctcggagcgcgtgggcaagtcgggggccgagggcagccgcctgcgggaggcgcagcacatcaacaagtcgctgtcggctctgggggacgtcattgctgccctgcgctcccgccagggccacgtgcccttccgcaactccaagctcacctacctgctgcaggattcgcttagtggtgacagcaagaccctcatggtggtacaggtgtcccccgtggagaagaacactagcgagacgctctattccctcaagtttgctgagagggtgcgctctgtggagctggggcctgggctacgcagggcagagcttgggtcctggtcaagccaggagcatctagagtgggagccggcttgtcagacgccacagccctcggcacgggcccactcagcccccagctctgggaccagtagccgccctggatccatccggaggaagctgcagccctcggggaagtcgcggccactgcctgtgtgacggatgtgaccccgctgggcctgaagctgggccctcactggcctgtccctgctgcagcgccaggacccccggaggtagaggcgagagtggaggctcttcttctgccccgtctcccctcaaagatgagaaacatgttcagaaggaaacggtgtctctcggctgtggctctgagtgcaaattgcatgggcggaaaggcgggggtggctgctcttcctggcaggcctgggccatcagcgaactgggccccgtgaggagggcgggagtgtggaggagggtgggcctctcacccaggctttctcggcccctctcctcagcttgcagagctggccagccccctccttagggggtgggcgaggagcctctgggcagacccaagaaccatggggactggggtgggttggtggcaccaatggcagccctccccgcccctctccttcaaggagggttcccgcagctggggggtgtgcggaggcgcatggcctcccgccacggggccgtgctgtgtttatggctggcagaggcagccagcgggtgggggattctgctgctcgctcacctgcctggctcgctggtctctcgaattttcttccctctgaaatcctatttaagaacttttggaagcttagccatttttacttattaaaataaaagaagcctttttacacaa

SEQ ID NO: 95 - DNA, 1804 bp, Homo sapiens
  source 1..1804: mol_type = genomic DNA
agacacactctgccccaaccatcctgaagctacaggtgctccctcctggaatctccaatggatttcagtcgcagaagcttccacagaagcctgagctcctccttgcaggcccctgtagtcagtacagtgggcatgcagcgcctcgggacgacacccagcgtttatgggggtgctggaggccggggcatccgcatctccaactccagacacacggtgaactatgggagcgatctcacaggcggcggggacctgtttgttggcaatgagaaaatggccatgcagaacctaaatgaccgtctagcgagctacctagaaaaggtgcggaccctggagcagtccaactccaaacttgaagtgcaaatcaagcagtggtacgaaaccaacgccccgagggctggtcgcgactacagtgcatattacagacaaattgaagagctgcgaagtcagattaaggatgctcaactgcaaaatgctcggtgtgtcctgcaaattgataatgctaaactggctgctgaggacttcagactgaagtatgagactgagagaggaatacgtctaacagtggaagctgatctccaaggcctgaataaggtctttgatgacctaaccctacataaaacagatttggagattcaaattgaagaactgaataaagacctagctctcctcaaaaaggagcatcaggaggaagtcgatggcctacacaagcatctgggcaacactgtcaatgtggaggttgatgctgctccaggcctgaaccttggcgtcatcatgaatgaaatgaggcagaagtatgaagtcatggcccagaagaaccttcaagaggccaaagaacagtttgagagacagactgcagttctgcagcaacaggtcacagtgaatactgaagaattaaaaggaactgaggttcaactaacggagctgagacgcacctcccagagccttgagatagaactccagtcccatctcagcatgaaagagtctttggagcacactctagaggagaccaaggcccgttacagcagccagttagccaacctccagtcgctgttgagctctctggaggcccaactgatgcagattcggagtaacatggaacgccagaacaacgaataccatatccttcttgacataaagactcgacttgaacaggaaattgctacttaccgccgccttctggaaggagaagacgtaaaaactacagaatatcagttaagcaccctggaagagagagatataaagaaaaccaggaagattaagacagtcgtgcaagaagtagtggatggcaaggtcgtgtcatctgaagtcaaagaggtggaagaaaatatctaaatagctaccagaaggagatgctgctgaggttttgaaagaaatttggctataatcttatctttgctccctgcaagaaatcagccataagaaagcactattaatactctgcagtgattagaaggggtggggtggcgggaatcctatttatcagactctgtaattgaatataaatgttttactcagaggagctgcaaattgcctgcaaaaatgaaatccagtgagcactagaatatttaaaacatcattactgccatctttatcatgaagcacatcaattacaagctgtagaccacctaatatcaatttgtaggtaatgttcctgaaaattgcaatacatttcaattatactaaacctcacaaagtagaggaatccatgtaaattgcaaataaaccactttctaattttttcctgtttctgaattgtaaaaccccctttgggagtccctggtttcttattgagccaatttctggg

SEQ ID NO: 96 - DNA, 820 bp, Homo sapiens
  source 1..820: mol_type = genomic DNA
actcgccacctcctcttccacccctgccaggcccagcagccaccacagcgcctgcttcctcggccctgaaatcatgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggctgagtgcacaggtgccgccagctgccgcaccagcccgaacaccattgagggagctgggagaccctccccacagtgccacccatgcagctgctccccaggccaccccgctgatggagccccaccttgtctgctaaataaacatgtgccctcagg

SEQ ID NO: 97 - DNA, 1682 bp, Homo sapiens
  source 1..1682: mol_type = genomic DNA
ggagggaagccggcccaggctcggtgaggaggcaaggttctgaggggacaggctgacctggaggaccagaggcccccggaggagcactgaaggagaagatctgccagtgggtctccattgcccagctcctgcccacactcccgcctgttgccctgaccagagtcatcatgcctcttgagcagaggagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtccgagttccaagcagcactcagtaggaaggtggccgagttggttcattttctgctcctcaagtatcgagccagggagccggtcacaaaggcagaaatgctggggagtgtcgtcggaaattggcagtatttctttcctgtgatcttcagcaaagcttccagttccttgcagctggtctttggcatcgagctgatggaagtggaccccatcggccacttgtacatctttgccacctgcctgggcctctcctacgatggcctgctgggtgacaatcagatcatgcccaaggcaggcctcctgataatcgtcctggccataatcgcaagagagggcgactgtgcccctgaggagaaaatctgggaggagctgagtgtgttagaggtgtttgaggggagggaagacagtatcttgggggatcccaagaagctgctcacccaacatttcgtgcaggaaaactacctggagtaccggcaggtccccggcagtgatcctgcatgttatgaattcctgtggggtccaagggccctcgttgaaaccagctatgtgaaagtcctgcaccatatggtaaagatcagtggaggacctcacatttcctacccacccctgcatgagtgggttttgagagagggggaagagtgagtctgagcacgagttgcagccagggccagtgggagggggtctgggccagtgcaccttccggggccgcatcccttagtttccactgcctcctgtgacgtgaggcccattcttcactctttgaagcgagcagtcagcattcttagtagtgggtttctgttctgttggatgactttgagattattctttgtttcctgttggagttgttcaaatgttccttttaacggatggttgaatgagcgtcagcatccaggtttatgaatgacagtagtcacacatagtgctgtttatatagtttaggagtaagagtcttgttttttactcaaattgggaaatccattccattttgtgaattgtgacataataatagcagtggtaaaagtatttgcttaaaattgtgagcgaattagcaataacatacatgagataactcaagaaatcaaaagatagttgattcttgccttgtacctcaatctattctgtaaaattaaacaaatatgcaaaccaggatttccttgacttctttgagaatgcaagcgaaattaaatctgaataaataattcttcctcttca

SEQ ID NO: 98 - DNA, 358 bp, Homo sapiens
  source 1..358: mol_type = genomic DNA
cctttctctctcgcgcgcggtgtggtggcagcaggcgcagcccagcctcgaaatgcagaacgacgccggcgagttcgtggacctgtacgtgccgcggaaatgctccgctagcaatcgcatcatcggtgccaaggaccacgcatccatccagatgaacgtggccgaggttgacaaggtcacaggcaggtttaatggccagtttaaaacttatgctatctgcggggccattcgtaggatgggtgagtcagatgattccattctccgattggccaaggccgatggcatcgtctcaaagaacttttgactggagagaatcacagatgtggaatatttgtcataaataaataatgaaaaccta

SEQ ID NO: 99 - DNA, 2420 bp, Homo sapiens
  source 1..2420: mol_type = genomic DNA
agtccccctcccttcgcaagcacgctgtcccccttctcccacacggctgcggagcctgggctgggcgcgggggaggcggagggcatcgcggaggggatcccgccccctcccccggccgctcggtgcccccacacccacgtgagcgcagggccggcccggatccctcccgcagcatagcgcgtcccggctccgcgccggtgcctccacggtccggtccccgcgccggtgctgcacagtccctggcgggtccccgcggccccggccgggcgcttcgccgggctccggctcctgcatccgggcgcagcgcgcaggccgaggcgcgggcaggccgcccccgccgctccggacgccggtgagtggggactccgcggctcggcgcggggactcggtgtggctgcggcgcggaggttgcgggcaggtgggggatccgaggcgctggggtcaggcgtgtggggcgctccctgcgcccacgtccccacttctccgccgccctttgtctctgagctcctgcttgccccttagagccgttgccaccgcttttctcctggtcgttctgatagaccatgagcagccatggcaacagcctgttcctgcgggagagcggccagcggctgggccgggtgggctggctgcagcggctgcaggaaagcctgcagcagagagcactgcgcacgcgcctgcgcctgcagaccatgaccctcgagcacgtgctgcgcttcctgcgccgaaacgccttcattctgctgacggtcagcgccgtggtcattggggtcagcctggcctttgccctgcgcccatatcagctcacctaccgccagatcaagtacttctcttttcctggagagcttctgatgaggatgctgcagatgctggtgttacctctcattgtctccagcctggtcacaggtatggcatccctggacaacaaggccacggggcggatggggatgcgggcagctgtgtactacatggtgaccaccatcatcgcggtcttcatcggcatcctcatggtcaccatcatccatcccgggaagggctccaaggaggggctgcaccgggagggccggatcgagaccatccccacagctgatgccttcatggacctgatcagaaatatgtttccaccaaaccttgtggaggcctgcttcaaacagttcaagacgcagtacagcacgagggtggtaaccaggaccatggtgaggacagagaacgggtctgagccgggtgcctccatgcctcctccattctcagtggagaacggaaccagcttcctggaaaatgtcactcgggccttgggtaccctgcaggagatgctgagctttgaggagactgtacccgtgcctggctccgccaatggcatcaacgccctgggcctcgtggtcttctctgtggcctttgggctggtcattggtggcatgaaacacaagggcagagtcctcagggacttcttcgacagcctcaatgaggctattatgaggctggtgggcatcattatctggtatgcacctgtgggcatcctgttcctgattgctgggaagattctggagatggaagacatggccgtcctggggggtcagctgggcatgtacaccctgaccgtcatcgtgggcctgttcctccatgccggcattgtccttcccctcatctacttcctcgtcactcaccggaaccccttccccttcattgggggcatgctacaagccctcatcaccgctatgggcacgtcttccagctcggcaacgctgcccatcaccttccgctgcctggaggagggcctgggtgtggaccgccgcatcaccaggttcgtcctgcccgtgggcgccacggtcaacatggatggcactgccctctacgaggccctggctgccatcttcattgctcaagttaacaactacgagctcaacctgggtcagatcacaaccatcagcatcacggccacagcagccagtgttggggctgctggcatcccccaggcgggtctggtcaccatggtcattgtgcttacgtcggtcggcttgcccacggaagacatcacgctcatcattgccgtggactggttccttgaccggcttcgcacaatgaccaacgtactgggggactcaattggagcggccgtcatcgagcacttgtctcagcgggagctggagcttcaggaagctgagcttaccctccccagcctggggaaaccctacaagtccctcatggcacaggagaagggggcatcccggggacggggaggcaacgagagtgctatgtgaggggcctccagctctgcccccccagagaggagggaagggggctggggaggggagtcctggtgacacatctgttgcccaactgaccgtgggctgaacacacgttctgcttgactcatttaggggggagggaaaagtgaaataaaggagcaggaatgaaaggta

SEQ ID NO: 100 - DNA, 8558 bp, Homo sapiens
  source 1..8558: mol_type = genomic DNA
gaggccctgacgtggacacacttcgggtttcacgactccgggtttctccaggggatgggccggccggtagaggcgcgtgagggagacgaagggacttccgtttccttcacctaggctggggccaagccgcagagcggagttggcatttccagattggggctcgggccgcgcctcctccgggaccctccccttggaccgagccgatcgccgcggggcagttcgggccggctgtcctggcgcgaaaaggtggacaagtcctattttcaagagaagatgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtcttaatctaactctatagtaggcatgttatgttgttcttattaccctgattgaatgtgtgatgtgaactgactttaagtaatcaggattgaattccattagcatttgctaccaagtaggaaaaaaaatgtacatggcagtgttttagttggcaatataatctttgaatttcttgatttttcagggtattagctgtattatccattttttttactgttatttaattgaaaccatagactaagaataagaagcatcatactataactgaacacaatgtgtattcatagtatactgatttaatttctaagtgtaagtgaattaatcatctggattttttattcttttcagataggcttaacaaatggagctttctgtatataaatgtggagattagagttaatctccccaatcacataatttgttttgtgtgaaaaaggaataaattgttccatgctggtggaaagatagagattgtttttagaggttggttgttgtgttttaggattctgtccattttcttttaaagttataaacacgtacttgtgcgaattatttttttaaagtgatttgccatttttgaaagcgtatttaatgatagaatactatcgagccaacatgtactgacatggaaagatgtcaaagatatgttaagtgtaaaatgcaagtggcaaaacactatgtatagtctgagccagatcaaagtatgtatgtttttaatatgcatagaacaaaagatttggaaagatatacaccaaactgttaaatgtggtttctcttcggggagggggggattgggggaggggccccagaggggttttataggggccttttcactttctacttttttcattttgttctgttcgaattttttataagtatgtattacttttgtaatcagaatttttagaaagtattttgctgatttaaaggcttaggcatgttcaaacgcctgcaaaactacttatcactcagctttagtttttctaatccaagaaggcagggcagttaacctttttggtgccaatgtgaaatgtaaatgattttatgtttttcctgctttgtggatgaaaaatatttctgagtggtagttttttgacaggtagaccatgtcttatcttgtttcaaaataagtatttctgattttgtaaaatgaaatataaaatatgtctcagatcttccaattaattagtaaggattcatccttaatccttgctagtttaagcctgcctaagtcactttactaaaagatctttgttaactcagtattttaaacatctgtcagcttatgtaggtaaaagtagaagcatgtttgtacactgcttgtagttatagtgacagctttccatgttgagattctcatatcatcttgtatcttaaagtttcatgtgagtttttaccgttaggatgattaagatgtatataggacaaaatgttaagtctttcctctacctacatttgttttcttggctagtaatagtagtagatacttctgaaataaatgttctctcaagatccttaaaacctcttggaaattataaaaatattggcaagaaaagaagaatagttgtttaaatattttttaaaaaacacttgaataagaatcagtagggtataaactagaagtttaaaaatgcttcatagaacgtccagggtttacattacaagattctcacaacaaacctattgtagaggtgagtaaggcatgttactacagaggaaagtttgagagtaaaactgtaaaaaattatatttttgttgtactttctaagagaaagagtattgttatgttctcctaacttctgttgattactactttaagtgatattcatttaaaacattgcaaatttattttatttatttaattttctttttgagatggagtcttgcttgtcacccaggctggagtgcagtggagtgatctctgctcactgcaacctccgccttctgggttcaagcgattctcgtgcctcagcttcctgagtagctggaattacaggcaggtgccaccatgcccgactaatttttttttatttttagtagagacggggtttcaccatgttggccaggctggtatcaaactcctgacctcaagagatccactcgccttgccctcccaaagtgctgggattacaggcttgagccaccacgcccggctaaaacattgcaaatttaaatgagagttttaaaaattaaataatgactgccctgtttctgttttagtatgtaaatcctcagttcttcacctttgcactgtctgccacttagtttggttatatagtcattaacttgaatttggtctgtatagtctagactttaaatttaaagttttctacaaggggagaaaagtgttaaaatttttaaaatatgttttccaggacacttcacttccaagtcaggtaggtagttcaatctagttgttagccaaggactcaaggactgaattgttttaacataaggcttttcctgttctgggagccgcacttcattaaaattcttctaaaacttgtatgtttagagttaagcaagactttttttcttcctctccatgagttgtgaaatttaatgcacaacgctgatgtggctaacaagtttattttaagaattgtttagaaatgctgttgcttcaggttcttaaaatcactcagcactccaacttctaatcaaatttttggagacttaacagcatttgtctgtgtttgaactataaaaagcaccggatcttttccatctaattccgcaaaaattgatcatttgcaaagtcaaaactatagccatatccaaatcttttccccctcccaagagttctcagtgtctacatgtagactattccttttctgtataaagttcactctaggatttcaagtcaccacttattttacattttagtcatgcaaagattcaagtagttttgcaataagtacttatctttatttgtaataatttagtctgctgatcaaaagcattgtcttaatttttgagaactggttttagcatttacaaactaaattccagttaattaattaatagctttatattgcctttcctgctacatttggttttttcccctgtccctttgattacgggctaaggtagggtagagtgggtgtagtgagtgtatataatgtgatttggccctgtgtattatgatattttgttatttttgttgttatattatttacatttcagtagttgttttttgtgtttccattttagtggataaaatttgtattttgaactatgaatggagactaccgccccagcattagtttcacatgatataccctttaaacccgaatcattgttttatttcctgattacacaggtgttgaatggggaaaggggctagtatatcagtaggatatactatgggatgtatatatatcattgctgttagagaaatgaaataaaatggggctgggctcagtggctcacgcctgtaatcccagcactttgggaggctgaggcaggtggatcacgaggtcaggagatcgagaccatcctggctaacacggtgaaaccccgtctctactaaaaaacagaaaattagccgggcgtggtggcgggcgcctgtagtcccagctactcgggaggctgaggcaggagaatggtgtgaacccgggaggcagagcttgcagtgagccgagatctcgccactgcactccagcctgggcaacagagcaagactctgtctcaaaaaaaaaaaaaaaagaaataagaaaatgggaagcaatatttgacatagttctttttagtcaaatctacttgttaaaaaaagggtagcagtttattcatctgtgaaaggaaaataatacttatcttacaaggttgcaagagctcaaggagaccatgtatgtaaagttcctgctgtaaatatgaactcccatcctaatacccttttacctctctgtgggtttgtcttgacctggaaatttgggctaaaacttagaaaaaattcttacatgataactcagtgatgcttactcatagtttttggtgtttctcatagataagatataaatcagctgggcgcggtggctcatgcctgtaatcccagcactttgggaggccgaggcgggcagatcacctgaggtcgggaggtcgagaccagcctgaccaacatggagaaaccccgtctctactaaaaatacaaaattagctgggcgtggtggctcatgcctgtaatcccagctacttgggaggctgaggcaggagaatcgcttgaacccaggaggcggaggttgtggtgagcgaagatcgtgccattgcactccagcctgggcaacaagagcaaaactctgtctcaaaaaaaaaaaaagatataaatcacaataaataaataggtcaatacaaatgttagccaggcgtggtggcacatgcccatagtcgcagctactctggaggcagaggcaggaggatcacttgagcccatgaatttgaggcagcagtgagctatgattgtgccactgtactccagtctgggtgacagagtgagaccccatctctaaataaataggtcaaacccttaaaaatatttaaattcttaaaaaattgaaaagattattcttctcaaatttagttgagctttctaagagaagcaattggctttttcccacttcaataatcattttcagtttgactcatacagttaacacaatgtgaatttcttcctcagcataacagagttatagaatgacagggctggaagtgaccttagagagtatccagttctttcattttacaggtgaggcaactgagactcaaaggtgatgtaatttgtgcaaagattatagctaattagtagcagagccctgactgggacatagtttgaaggtgaaaaacttcaccaagctacctttcttgaaaggtccaaatgtttatgttttcaactactctttccactgtaccataactttcactacatattaaatgacactttataactaatataataggacaatcatcaatgcatatatagccagcccttcatatctgtgggttttgcatccatggattcaaccaaggaggaattgaaaacactgagaaaaaaaaaaaagaccacacaataaaaaaaaaaaatacaaaataatacaaagaaaaagccaaaattgtcatactgttgttaagcaacagtataacaactatttacatagcattaaggttggtgcaaaaatgcaaaaaaaaaaaaagcaattatttttaaaccaacctaatatattgtattaggtattaaagtcatctggacatgaattaaagtatatgatgccagcctggacaaaaggcaaaaccctgtctctacaaaaaatacaaaaattagctgggcatggtggtgtgtgcctgtagtcctggctactccggagcctgaggtgggaggatcgcttgagtctgggaggcagaggctgcattgagctatgatcatggcactgcattccagcctgggtgacagtgcaagaccttgtctcagaataaataaagtatgtgatgaagatgtgcatacattatatgcaaatactgttttttttttttttaatttaaacagtctcactgtgttgcccaggatggagtgcaatggcacaatcttggctcatggcaaactctgcctcgcaagcagctgggactacaggcatgctccacggtgcccagttaattttttttgtattcttagtagagacagggtttcaccatgttggccaggctagtcttgaatttctgacctcaagtgattcatctcccaaagtgctgggattacaggcgtgagccaccacggccggctaatttttgtattttttagtagtgactggtttcgcggtgttgaccaggctggtctcgaactcctgatctcaggtgatctgcctgcctcggcctcacaaagtgctgggattacaggtgtgaaccactgctcccggccttgtgtgattttatctaagggacttaagcgtcctcaggtcctagggggtcgtgaaaccaaaaccccagggatagcaagggacaattgtatcttcaaagtagacaaatggcgccgggcacggtggctcacgcctgtaatcccagcagtttccgaggctgaggcaggcggctcacctgaggtcaggagttggagaccagcctggccaacatgctgaaaccctgtctgtacaaaaatacaaaaatagctgggcatggtggcgcatgcctgtagtcccagctactagagcgactgaggcaggagaattgcttgaacctgggaggcggaggttgcagggagccaagatggcgccaccgcactccagcctaggtgatagagtgagactccctctcaaaaacaaaacaaaacaaaaaaattagacaaatgctacattaatgtttgggtggtcagattctactttgaatctgaagtttgcagatatgcctatagatttttggagtttaccactttcttattctgtatcattaatgtaatattttaaattactatatatgttaccatttttctggatttagtaagaaatttgcagttttggtttgatgtaacaagggttttaatgtaatttatgttagattttgcatttttttcattactgttatattttaacctgactgactgatctaattgtattagtattgtgaataatcatgtgaaatgttttgagacagagtactatatttgtgaatataattttatggtttttttcacttagaacctttctgtgtggaaaactaagaaaattgctttctgctgtataatctggcattcattgtagattaaagcttatttttctgtgaataaaacgtattcaataaaatactattctttaaaatta

SEQ ID NO: 101 - DNA, 1889 bp, Homo sapiens
  source 1..1889: mol_type = genomic DNA
gcacttggcttcaaagctggctcttggaaattgagcggagagcgacgcggttgttgtagctgccgctgcggccgccgcggaataataagccgggatctaccatacccattgactaactatggaagattataccaaaatagagaaaattggagaaggtacctatggagttgtgtataagggtagacacaaaactacaggtcaagtggtagccatgaaaaaaatcagactagaaagtgaagaggaaggggttcctagtactgcaattcgggaaatttctctattaaaggaacttcgtcatccaaatatagtcagtcttcaggatgtgcttatgcaggattccaggttatatctcatctttgagtttctttccatggatctgaagaaatacttggattctatccctcctggtcagtacatggattcttcacttgttaagagttatttataccaaatcctacaggggattgtgttttgtcactctagaagagttcttcacagagacttaaaacctcaaaatctcttgattgatgacaaaggaacaattaaactggctgattttggccttgccagagcttttggaatacctatcagagtatatacacatgaggtagtaacactctggtacagatctccagaagtattgctggggtcagctcgttactcaactccagttgacatttggagtataggcaccatatttgctgaactagcaactaagaaaccacttttccatggggattcagaaattgatcaactcttcaggattttcagagctttgggcactcccaataatgaagtgtggccagaagtggaatctttacaggactataagaatacatttcccaaatggaaaccaggaagcctagcatcccatgtcaaaaacttggatgaaaatggcttggatttgctctcgaaaatgttaatctatgatccagccaaacgaatttctggcaaaatggcactgaatcatccatattttaatgatttggacaatcagattaagaagatgtagctttctgacaaaaagtttccatatgttatatcaacagatagttgtgtttttattgttaactcttgtctatttttgtcttatatatatttctttgttatcaaacttcagctgtacttcgtcttctaatttcaaaaatataacttaaaaatgtaaatattctatatgaatttaaatataattctgtaaatgtgtgtaggtctcactgtaacaactatttgttactataataaaactataatattgatgtcaggaatcaggaaaaaatttgagttggcttaaatcatctcagtccttatggcagttttattttcctgtagttggaactactaaaatttaggaaaatgctaagttcaagtttcgtaatgctttgaagtatttttatgctctgaatgtttaaatgttctcatcagtttcttgccatgttgttaactatacaacctggctaaagatgaatatttttctactggtattttaatttttgacctaaatgtttaagcattcggaatgagaaaactatacagatttgagaaatgatgctaaatttataggagttttcagtaacttaaaaagctaacatgagagcatgccaaaatttgctaagtcttacaaagatcaagggctgtccgcaacagggaagaacagttttgaaaatttatgaactatcttatttttaggtaggttttgaaagctttttgtctaagtgaattcttatgccttggtcagagtaataactgaaggagttgcttatcttggctttcgagtctgagtttaaaactacacattttgacatagtgtttattagcagccatctaaaaaggctctaatgtatatttaactaaaattactagctttgggaattaaactgtttaacaaataa

SEQ ID NO: 102 - DNA, 809 bp, Homo sapiens
  source 1..809: mol_type = genomic DNA
acactctaccaccatgaatccactcctgatccttacctttgtggcagctgctcttgctgccccctttgatgatgatgacaagatcgttgggggctacaactgtgaggagaattctgtcccctaccaggtgtccctgaattctggctaccacttctgtggtggctccctcatcaacgaacagtgggtggtatcagcaggccactgctacaagtcccgcatccaggtgagactgggagagcacaacatcgaagtcctggaggggaatgagcagttcatcaatgcagccaagatcatccgccacccccaatacgacaggaagactctgaacaatgacatcatgttaatcaagctctcctcacgtgcagtaatcaacgcccgcgtgtccaccatctctctgcccaccgcccctccagccactggcacgaagtgcctcatctctggctggggcaacactgcgagctctggcgccgactacccagacgagctgcagtgcctggatgctcctgtgctgagccaggctaagtgtgaagcctcctaccctggaaagattaccagcaacatgttctgtgtgggcttccttgagggaggcaaggattcatgtcagggtgattctggtggccctgtggtctgcaatggacagctccaaggagttgtctcctggggtgatggctgtgcccagaagaacaagcctggagtctacaccaaggtctacaactatgtgaaatggattaagaacaccatagctgccaatagctaaagcccccagtatctcttcagtctctataccaataaagtgaccctgttctcac

SEQ ID NO: 103 - DNA, 3145 bp, Homo sapiens
  source 1..3145: mol_type = genomic DNA
atagaccaggcttctcaggagaaaccccgggagattccacactgtcagccccttctccaagatcagtacgtgggcctgactcctcctcggtgcccagctcagtattggcaactaggagagtagtgagattgaacttggccttgaggaacagctgcctctagagttggatcagacaagggtgctgagagccgggactcacaaccaaaggagaaatgagcaatccgcggtctttggaagaggagaaatatgatatgtcaggtgcccgcctggccctaatactgtgtgtcaccaaagcccgggaaggttccgaagaagacctggatgctctggaacacatgtttcggcagctgagattcgaaagcaccatgaaaagagaccccactgccgagcaattccaggaagagctggaaaaattccagcaggccatcgattcccgggaagatcccgtcagttgtgccttcgtggtactcatggctcacgggagggaaggcttcctcaagggagaagatggggagatggtcaagctggagaatctcttcgaggccctgaacaacaagaactgccaggccctgcgagctaagcccaaggtgtacatcatacaggcctgtcgaggagaacaaagggaccccggtgaaacagtaggtggagatgagattgtgatggtcatcaaagacagcccacaaaccatcccaacatacacagatgccttgcacgtttattccacggtagagggatacatcgcctaccgacatgatcagaaaggctcatgctttatccagaccctggtggatgtgttcacgaagaggaaaggacatatcttggaacttctgacagaggtgacccggcggatggcagaagcagagctggttcaagaaggaaaagcaaggaaaacgaaccctgaaatccaaagcaccctccggaaacggctgtatctgcagtagaagtagaaagaccaggaggagctttccttccagcattctttctgtctcacagaaatttagaggcagctcttacctctccccaagatcttctgttcccaaggccaaatggcacccagtttcttttccatcacacccttcatgcaggtcctcctgtccttattagagcaagccagccaaaacttagcacaaggcatggtggcaacattaacatcacctccctcaggctggactttctatctttattaatgcaaccgaagagacctaagagtgcattcacttatcccactttctgttcctgtggtcttctttctcccatgaagcagaaactggataaagctcaagattttccatagacaaaccaaagcccactcatcccctcctaccccaatccaacctctgctggctcctgcatctcacttggaggtcaaacctcctcctgaggccaatgcattcccaacttccagttctttcctttaccctggagagttagtaaggtaagaaccattctttctctccaaaaccactcctccttggctggcaagttggtgtcctaactccgttctcttcctagctcatggcctctctagataataaagttgtctcctcctttctggatctcttcctcctaacacccctcccctgaaaccctggactctgccctctctccaagaaaatccatctattcaactattcttgcattcaattactctaaatgagagcgtgttggagctatggcaaattccctgttgtcaccttgctattttgcagacaacataatatttaacctctcataaccagagaggttaaataatttgtcaaatgcaatacagtaagacagaggcaaggacaaggtttgacttccagcccagcctcttttccacaacctgctaaatcctgatccatctgaaaacttttctaattagtgaagatgactaataaaaattttccctatctccaaggtaggagctttctggaagtttctagaaattttcaataaccaccagccaaggttacctccaggtaaccttgcagcaccaggctggaagtcagatcggcttcactatcttccaactctacagcctgtatctctccatccccagctttgacctttcctgctcaagtaacctacgggcacatccagcgtcactaaaaactcagggcttttcttcccggttactcctccaagcgttccctggtatcctcaacctcagatcccaggttcagatttctgcagtcaatctatgacccctctcttcttgcatccttcatatgccaccagacaccatgcccagtccagcctgattttgaaacaactttcatgccggtcttctcttccctgacatgttactgtccaggctcaagtcctcagcttctcatatctgcatctttgcaaccaacttcctcccttgcctctctgcttttccatcccacttttcatgtgtcctccataccatctataacagtgatctccctggaacactcaagaagacacaacataccatattatttaaagaccagggtactggacagtggctcacacctgtattcccgactttgagagtctgaagcgggaggatcacttgaggccaggagttaagagaccagcctgggcaacacagcaagaccctgtctctaaaaaaaaaaattaattaactgggtatggtggcacatgcctgtagtcccagctactcaggaggctgaggtgggaggatgacttgagcccaggagtttgaggctgcaaggagctatgatcatgccagtgcatcccagctctaggtgagacagtgagatccggtctccaaaataaatcaatcaatcaaataaagaccaaagtcaaaccgcacatcaggatctctcacacccttccaattttgccatctaccagcacttagctaaacccatctcccatctcttccaccatgaattcactctttcaaaaaggctaatgtcttcttactcacccttgcctctaagcctttgctatcaccatttcccccaagctggagggccctccctctccctttacccctcttccactacctcccacccctactttttccagaaagccatttcctctcttttttctgattgatccttccctctcacccaggattagatgctggaaatgaccacttctggagggcagggaacaagcccttaatctgcataatgagtgttcaataaacagttgtcaaactttgaaa

SEQ ID NO: 104 - DNA, 5125 bp, Homo sapiens
  source 1..5125: mol_type = genomic DNA
agaagcaggttggctgtgatgacagcacagagctcaggaacgctgcctgaggaccctggggcctacgaggaggagaagagggcaggagctggtggggtgcttgcagagaccctgggctcctatcctgccataagcctcgctgtctcctgatatctgcagccaggccctactgacacccccaggcctgagtgcaagcagagaccccaccattcccaggccctggaggactggtccaccttaactgggcagcccttggggcaggcgctggccggtgcctcagcccaggcctctgtgctctgcatgcactgccagcctgccatcaggcctctattgcagccctgaaccatgatccagggcaccttggagccagatggtcccctctggggctgggactgggacagtgacaatgactgggatagtgctgtgctggccctcctggcgctggctgtggtggctgccacagcgctggccttacactggtttggctccgggcacgatcaagaggcggcagaaccggtgtccacagccctcggggctcaacctcatcaggcaggaggagctgagctggccctgcaaccgaagtctaaggtcagtgatggcagcgaggggcagagcccagggcaggggaaaccagagcccccaggacgcggccagcagagccctgtccctgctgcagcgccgggcgggggcctggccgccatggcccggcttccactcaagacggctgtcgaggaggcccgcagagaggcattaggacagcaacggggcagtgccacccccgcggccccccgagcggaaggaaaggagcctcccaggccaggcactgccctcctgggcaggagcgaagcaggggggatgtccgcccccctcctgatccacttcactcctcggagccctggcagcgaagcggaggcggagacaggtggtgtcagggcgtcctctcgccaggccgcaggccccgcggggcaacaggacactggcccctggcaggcgggcgcggggccctcgggctcgatggggagaggccggggccggcggcggcggatggacgctggctcgggagacagagcccgccgcccccggaaactggacccgctccgcctgggcgccgcggggagcgtgtgggacgcggtggacggggccgccgccctggacgcccacgcgcgcggcctccccacaggacccccactcgcccaggagcccgcactcccggcgctgcccgctccccgcgccctgcagcctgggtctcagacggaaggctctggggccaagggtggctggagcagggaggcctcgggggtccctgcccccggaggaggctggccctgggtcagcagggaggtcccgggcacccggagctttggcccagccccagactccacgcgcccctggctagagagtccgcctcaaggtcgcccactctcgtcccaagggccgggtgccacaggggcctacgatgccggcgaggccggggctgacagctcccgagataacagtcctgccgctgacctggggcccacccggcccccggagcaagcaaagccggctgcagccggccacagccgcgcgccctcccggagccgtgagcctcgcccgcgctccgcctccccgcccgcagctcccggcccggggttcccacctgaagccctgactctcccctctccttcagactttttgcccctggaggttacccaggatccttccgtgggcgaaaatctcagagcggcgccagccccaagttcagcctcagcccaagtcttaacttcagctccagcctcagtcctagccccagccctggcttcatcccccagctcagcaccaacctcagccaccacctcaacctcatcccccacctcagccccagccccagctccaacctcagctccaacttcaaccccagccccagccccaagtccagctgcagccgcaactccagccccagccccagtcccagtcccaaccctcacacccccatccccagccctaaccccagtcccaaccccagccctaagcccagctccaactccagccctaaccccagccgcatccccagccctaaccccagtcccaaccccagccctaagcccagctccaactccagccccaaccccagccgcatcccctgccccagcccccacctcagccccaaccccaaccccagccgcatcccctgccccagctgacgggtcaaagcctcaggagagtgtggctctccccaggcgctaccaggaggggcaggtctcagccagctggggaaaccttattgccatggttcttagaagccaccccttccccaggcaagacaggccccaagggagtgtcccgagggcggttcccgggagccccgtgggtcccagcacttccacacactctgaggacagacacggcccctcttcttcagtggggacagtcatagggacaggtacagggggcctggttgaggctggaggtcagccacagccaagaagctccgagaccaacggatcgcccagcccagaccctcccccaggcctaagaggagagggaaccagggagaaaagtctagacccgctgccccaagccgcgatgcccaggggccccgcacagccccccgcgcagaggccgcctggccccgcggcctcctcctctgcgaggcgctcacagccggtaccccagctacggaaacgcagcaggtgcgaaatcgccccgagctcggagcaggaggtcaggccggccgcctcgggggaccctcaaggggaggcgccgggggaggggggcagccctgccggccgcagcggggcgctcacggaaaagcaggaggaggcccggaagctcatggtgtttctgcagaggcccgggggttggggggtggtggaggggccccggaagcccagctcccgggccctggagcccgccacggcggcagccctgcggcggcggctggacctgggcagttgcctggacgtgctggcctttgcccagcagcacggagagcccggcctggcgcaggagacctacgcgctgatgagcgacaacctgctgcgagtgctgggagacccgtgcctctaccgccggctgagcgcggccgaccgcgagcgcatcctcagcctgcggaccggccggggccgggcggtgctgggcgtcctcgtactgcccagcctctaccaggggggccgctcagggctccccaggggccctcgtggcgaggagcctcctgcggcggcccctgtgtccctgcctctacctgcgcacctgcatgtgttcaacccccgggagaacacctggcggcccctgacccaggtgcccgaggaggccccgcttcggggctgcggtctctgcaccatgcacaactacctgtttctggcggggggcatccgtggctccggtgccaaggccgtctgctccaacgaggtcttctgctacaaccctctgaccaacatctggagccaggttcggcccatgcagcaggcccgagcccagctcaagctggtggccctggacgggctgctctatgccatcggtggcgaatgcctgtacagcatggagtgctacgacccgcgaacagacgcctggaccccacgcgcgccactccccgcaggcaccttccctgtggcccacgaggctgtggcctgccgtggggacatctacgtcaccgggggtcacctcttctaccgcctgctcaggtacagccccgtgaaggatgcttgggacgagtgcccatacagtgccagccaccggcgttccagcgacatcgtggcactggggggcttcctgtaccgcttcgacctgctgcggggcgtgggcgccgccgtgatgcgctacaacacagtgaccggctcctggagcagggctgcctccctgcccctgcccgcccccgccccactgcactgcaccaccctgggcaacaccatttactgcctcaacccccaggtcactgccaccttcacggtctctggggggactgcccagttccaggccaaggagctgcagcccttccccttggggagcaccggggtcctcagtccattcatcctgactctgccccctgaggaccggctgcagacctcactctgagtggcaggcagagaaccaaagctgcttcgctgctctccagggagaccctcctgggatgggcctgagaggccggggctcagggaaggggctgggatcggaacttcctgctcttgtttctggacaactttccccttctgctttaaaggttgtcgattattttgaagcccagactccctcagcctctttctgcccctcactccacacccagactgtttcctgactcaattccgtacctacttacagaccctctcagcttgctgacacccccctgtctgtgggactccctattccctagagccagggactgatgcgtctccacagacaaggacttggctcgctggagctctgctgagccgagagaggagggggtagaaaacattcacacttcctatgctctgtcagcaggacagggagcaaaaacgtccccaggcaacgccctcgcctctgggactttctgcctgtcctaaggcctccccaggtaccaaccccgtagctatctgggtctgtttggcactgtggattctcaagggcctagaacccttgcctctgaaactggtccgctggtgcagccctgctgtctgcagctcctgcccatacccccagcccacaccaggccaggcccactccgggctcaccaccctctgcagccttgtggggctctcccagcccctccagaagcccaccccacttctcgccaacccccgatctctaaatgaggcctgagcgtcaccctagttctgcccctttttagctgtgtagacttggacgagacatttgacttccctttctccttgtctataaaatgtggacagtggacgtctgtcacccaagagagttgtgggagacaagatcacagctatgagcacctcgcacggtgtccaggatgcacagcacaatccatgatgcgttttctccccttacgcactttgaaacccatgctagaaaagtgaatacatctgactgtgctccactccaacctccagcctggatgtccctgtctgggccctttttctgttttttattctatgttcagcaccactggcaccaaatacattttaattcaccgaaagca

SEQ ID NO: 105 - DNA, 1992 bp, Homo sapiens
  source 1..1992: mol_type = genomic DNA
acacatggatattggatatctgcataggcagcttgctccacgccggtgcctacctgtgcagatgggaaggaaaggaaagtggcaaggaggcagagaaagcatctgtacccttacaatttggtgagacaagaatgtatgaattcccacaggtcaaattataatgaagaaaggaacctctcttgagtacaaagagctacctatggtggtctggagccggaggaccacagcatcaaaggatataagatgcatagccaactgaggaacctgagcaattaaagagatccacagttaagtcacacttaactggcacttgtggaagccccgcaaggcctgaaggagagctgacataggcaccccagagagccagaatctggatcccatcttaataaggccatgaacaccagtggagaagaggcagaaacaccaatggataaggaacattcacatctttcttcccatgtgcctctaagtgccagtgcaggccccacaggccaagctacagggagaaaggagatgacgcaaaggaacctaactggactttaatcactagaagtgagaagagaaatctattggaacctcccaagataatgccaagggtcaaagggtgcgcagatacataagaccatggaaataatatcagacaaaaagcagattagagcaattttctttttcgagttcaaaatgggttataaagcagcggagacaaaccgcaacatcaccaacgcctttggcccaggaactgctaatgaaggtacagtgcagtcactgttcaggaagttttgcaaaggagactagagccttgaagatgaggagcatagtgaccagccattggaagtcgacaaagaccaattgagaggaatcattgaagctgatcatcttacaactacacgagaagttgtcaaagaacgcaatgttgaccattgtgtggtcttttcgcatttgaagcaaattggaaaggtgaaaaacttgataagtgggtgccttgtgagctcagcaaaaatccaaaaaaataatcatttttaagtgttgtcttctcttattctacgcaacaacaataaccattttgcaatcggattgtgatgtgcaatgaaaagtggatttggggccgggcgcggtggctcacgcctgtaatctcagcactttggaaggccaaggcgggcagatcacgaggtcaggagatcaagaccgtcctggctaacacggtgaaaccccgtctctactgaaaatacaaaaaattagccgggtgtggtggctggcgcctgtagtcccagctacaggctgaggcaggagaatggcatgaacctgggaggcggagcttgcagtgagccgagaccgtgccactgcactccagcctgggcgacagagcgatactccgtcaaaaaaaaaaaaaaaaaaaaaaaaagacaagtggattttatatatggcaaccagcaatgaccagctcagtggctggactgagaagaagctccaaagcacttcccaaagccaaacttgcaccaaaaaaaaggtcagggtcactgtttggtggtctgctgctggtctgatccaccgctgctctctgaatcctggcaaaaccattacatctgagaagtatgctcaacaaatcaatgagctacgccaaaaactgcagcatctgcagctggcattggtcaacataacgggtccaattcttctccacgacaacgctcaactgcaccttgcgcaagcagcgcttcaaaagttgaacaaattgggctacatagtttttcctcatccgccatattcacctgacgtcttgccaactaactaccacttcttcaagtatctcaacaactttttgcagggaaaacacttccacaaccagcaggatgcagaacacgctttccaagagtttgtcgaatcctgacgcacagatttttatgctacaggaataaactaacttatttctcattggcaaaaatgtgttgattgtaatggttcctattttgatgaataaatgtgtgtttgagccta

SEQ ID NO: 106 - DNA, 3453 bp, Homo sapiens
  source 1..3453: mol_type = genomic DNA
agctacatccgtcacctgacacggccctaccaggaacagccgcgctcccgcggattctggtgctgctcgcgtccccgctcccctattccccttattttattcctggctcccctcgtcgaaagtcttccattcttcaaactagattatttaaaaatgaaaaaggaagaaaggaaagcgaggtcatctcattgctctatccgccaatcaggaggctgaatgtcagttttgaactaaaagccgctccgctcctcttctagatttggaaaacaagcgaaattaaactaaaccgctgcacgcctctgacgcgacatctggacacggcgcggcgctggcgctgccggagctgtcgacccggcctggcgccggactaggactatttgccacgacatttcaaaggattccaagagagaatattggtgtccatgctgtgatgattcctcagctcctctcatctgatctccgtcctggcccccatgactttctttgtggtagttagggtgtggtatgtgccactgaggcccacacctattgctgcaatttatagcactgatctgtcatcaataccacttgctgtcttggatgtgaagatgatttttcctgcagggattccctctacaaaattaaaaacactgggcatgtggaaataatattcatgctttaaattgtcttttctcttcactacaccaggggtccccaacccctaggccacagactgtggccctagtgtagtgaatagaaaagacaatttaaagcgtgaatattatttcctcatgcccagtgtttttaattttgtactggtctgtggcttgttagaaaccaggctgcacagcagaaggtgggcagcagatattgagaaaccacagaaagagagaagttaaataattttcctgtcaaagaccacatcaatgatgaagccagaatttgagctcatgtacttaaccactggactacctgcctgccctgtcgaggaacagctaagtgtcccttttgatgagaagaataagcctcattctgattcaacagcagagatcaaagaaaagacttctgttttctggccaccagatatatgttatctgtgcttaaagaattgaaaaacacacatcaaaggagaattttcttggaaagagagggttcaagcatcactgttaggtgtgctggaatcctttcccgagtcagtactgctttctagaagaaaaccggggagatctatttggaatgtatctaactccaaagaaaccatcagaggtaacagtagagacggggtttcaccatgttggccagactggtcttgaactctcgacctcgtgattcgcccgcctcggcctcccaaagtgctgggattacaggtgtgagacaccacacccggcggatagagagaattttgacagtctctccaatgaacgccttcactgatatccaaagcatgaaggacacaccagggaaaaacatagacctaacacaggacaaatggaattattagaaacattttctagcagaagaacactattctgttgccatttgaatctttgcttctttctaggtttgacaatgagcctatcatataagcccaaatgtaaacagaaagaggttgaatcagtcacgataagcccaattatgctgtggtaacaaacaacctcaaaatctcattggcttaaaatatacagaattattcttactcatggcacatatccatctatcatctgcaggggatctgctcactgaagtcacttaggaacttggactgatggaacggccactttttggtcactatatgtattaatctgttttaatcctgctgataaagacccaaaattgggaacaaaaagaagtttaactagacttacagttccgcatggctgaggaggcctcagaatcatggtgggaggcgaaaggcacttcttacatggtggcagcaagagaaaaatgaggaagaagcaaaagcggaaacctctgataaacccatcagatcttatgagacttattccactatcaagagaatagcatgggaaagactggctcccataatttacctccctctgggtccctccctcaacatgtgggaattctgggagaaacaattcaagatatgacacattcataatttaaacagaagcctacgaagaactcataaattaaaagaagataatcttttcacaaggtgatggaggctttttattttgccacaaaaccactggtgacgttgcctgtggccaccttggagaagacactggaggcctgggacatggagactgcttttctgcagaaaccacatcccttggagtaatgagctacacctacctcaattattcagtgcagtacaacactccagacagggtctcactctgtcacccaggctggagtgtattggcatgattacagctcactgcaaccttgaactcccaggctcaaacctgagcagctgggactacagatgcaccaccatgcatggtaccagagatataataatgagaaacagacatgctccctcccctcattgaggttacagcttagtgtggagacacacagatgcctaacgcactatggtatggaaggtgctatggacacagtgctcaaatccatgatctacataggtggagaacttcagtagaggaagtggcaggaatttgggaatgaggagcacagtgattaaactggggccattcatatgagagtttaagaactcagaccagtgacttagattggcttctctcacatggcaagaaacattgctgctagcacttcccgagttctacgttctacaacatccaccactggatcttaacatagacgtaagatcaaatgcaatagcatgtcaaacaatgtgtaactccagttatacaaacattactgtatctcattggggatacgaagctctacacacttgaagatggtgaaggaatataaaaatctatgtctcacagtccagacttggagtacaagtaataagaagaataaaacttaatcccttaagtagattcaccataagttagctcagagcaattccagtgcaagtatggtctgtgatccagtagtatcttacagacagcaagttgaacattgtgggatgcatgagctattgaggcctttgcagctttctgctacatggaggctagggccagagtcaagatttatgctttgcagcacactggtcagctgtttttgcaaatcagattaaatgatttttaaatgaggctgagagcatgggagatactaatgtgtgtttccttgtgagctactgcataagttaggaaattgaaatacagaaagatgaaaagtgatttgcccaagcatatagatcaaagctgtggcagaaccaggactggaacctatatctctctactaatggtttttttaaaaaaataaccttgtttcaaaaatattaaaaagtcacaagaaaggtaaacatgtggataaacaaaatgaagaaaataaaaattatccagtaataacata

SEQ ID NO: 107 - DNA, 2046 bp, Homo sapiens
  source 1..2046: mol_type = genomic DNA
atcgttacccgagcccagcgccgggccaatgtcctatctcagggtttcccacgtcgctcgctccttgacggctgaccgacactgggtctgaggtgtaagcgctttccaggcccttccctggggcttggaggggaatggcgcaaaggacttttcttacaagcagagccactgcagttaccaaagagtatgtcgcttattgtctaagaggttggactcctgaaagcctggacagtgtcatcatcttcacctgaagagtggaaaaattcaagcaaagagtttctcaggaaatgaccaagtaaaacatctggagtaagtcagcaggattcacagtgactccccccaaccagctggtggtgtttctctctgaaagcagcgcttggtaccagtgtgaactctcttccttcagaagtctcatcaggtggacgtaatacgagggtctggtcccccataccttgaggactccgctgccatctgacaattacagacaatccaattgtcctcatccttgaggactctgctgccatctgacaattagagacagtccaaaacatgccacactttgatcctaaacacattcctggctttggtcacttttgaaaagttcctgagttatgagaaatcaataataagagtgttcaaaacctgaacactctttttagaaatacctgctggaactacatatatgatttatggggtactgtcttgtagtatctgggaaagtagacccatctaacctggaatgatcataggcaacaatgaccaaaaatggggatcttttgatatacctaaagtaatttatttgtgcacaccattggaaagagctggttttagaaccagacatgtaaataagagactgacttccactggcacctaaaaacttctaaaagaaatcctgaaagaaaaagtcaccttcattcaagaggaaattcatagaataggaccctgacaagtactcttgaatacaggtttctgataactttggagatcatatcattggactaagtaacaactttcaggatataattggagacacttgttattttactaaggatttgactggaatgacatattttcagatatgaccagactgctttgaagaatggaggttgactctatagaggcaattagaaagcctcttggaaagactggcctggtcccttgtctacgtagttccctttcaagatttctgtcctgtgttaagtaaagaatgtcactttctgacaggcctgggaacctcaaaatatttccgctgtgtatctctcattgttttatttcttctgagaaaactaaattcatggtacactgaagaggtgattccacaagtgatggcagctataaatcaatgactggtaggactacctgggcatgagcctttctggcaatgagggataccttgacactcagattttgatcatcagtgctttcaaaaggaaagatttttgatcaaaaggaagaaatgagaaaaagaacttttatctgaagaatgcaagtcctttccaattatcagatccagagaggtattaaaatgagacggcaatcctgtcctacacacacccccttttgaggcatgtactcatctttttatactgcttactattgccacaagtagctgaaaattagcctactggtgctgcaccagacattataacccatatcctatagcttaataatggatagccaatcaacagcttatgttatgttaatgtaaattattggtaaataactcaggaactgcctcttctttccttttaaaaacccacttgtgggccgggcgtggtggctcacgcctgtaatcccagcattttgggaggccaaggtgggcggatcacaaggtgaggagatcaagaccatcctggataacacagtgaaaccctgtctctactaaaaatacaaaaaattagccgggcgtggtggcgggcacctgtagtcccagctactcgggaggctgaggcaggagaaaggcgtgaacccaggaagcagagcttgcagtgagctgacatggcaccactgcactccagcccgggcaacagtgcgaggccctgtctc

SEQ ID NO: 108 - DNA, 8121 bp, Homo sapiens
  source 1..8121: mol_type = genomic DNA
attaggctaagggtgtggccagtcacaggttccccaggctggaagattcttcagacgtgggtccctctctctggtaggtctgggtttactagaatgcatctcaaactccaataagcaaggtctgacttttcagggagatcttaagggtaaggagggccagggagggtatttcacctgagatcccagccagtccttccaacagccagatgctgctactaatcccacagcacgttcaaagggacaaaaggggaccagtgacaactgattaaaggaaaatgtgatcctgagtgacccaggaccttgggactctgagggggaaccaaagagcttaactggtttaaaaacttaaaaggatcggccaggcttggtggctcacacctgtaatcccagcactttcagaggcccaggcgggtggatcacacgaggtcaggagtttgagaccagcctggccaatatggtaaaaccacgtctctaactgaaaatacaaaaattagccaggcgtggtggtgtgcacctgtagtcccagctacagggagggtgaagcaggacaaccacttggaccccagaagtggaggttgcagtttgcagagatcatgccactacactccagcctgggggacaaagtgagactgtctcaaaaaaacaaaaaagagagctttccaatgacacacacaccccgccccatttctcccccagcccagctcacagccaattactaaatagtggaaaggaggctacttaaaaagaagttccctattttcctgtgggggttactgggctttttagtaggccactcacccactaccctcgcaccaatcccccaaagtccgtaccctacttgaagattatgctgtaattatgggtgctgtgtttatagaccacagatttaacagaacaagcccacagaacacccatcttttttccccaatcaagatttttttattcacaaaagaaccacaacaatttagagcttctccatttttattacacaaaattgttaagttttcagcagtagggcttctcaaaacaccagctgcaggctattacctgaaaaagacaaggcagttatattaggttctcgtgtaaatatgaatatacaatcaagtcaagctcctgacaaattatacatcaaggatgtatatagttcaaagatattgtgctgttacctcccacctccccaaaactccaagaactagcacctgcagagaaaaggagaaatacagaaagagtcctgaagacagattagtagtcaaagcaaagacgccgcagggatttgaaccccgtcctggaaaccaggagtgccaaccaccagcatcttttgctttttttttttttaagctagaaaaaggccaaaaagcaaaacctgagaaaacaatacgtgttgttttctcaggaaaagaaaaaccttcatgaccctactgaagagcattggagatcagcttccgctaagatgctagcttggccaagtctgttatgttcacctgaaaaagtcttagcagagaatttttgcattcccacccaaaagccctctcagccactcaaatgcctatcttctccagtctacaagttacatgttcccacccagcattacagttcttgaacatgttatttctccacttactggtttaagttggttttagtcactggataagtattataatagagctacttagctgtggttttaatacccttctgttaacaatttgcaggcaaattaatggccttttctaacatagttcaacccaccaaagacctcgacaccatcgttaccttgaaaccgattatggatcatgcccacaaggatccaagctactggctgcatcgaggtgaggggtgaagggtctgtgctagatcaaaaggcacggggtggcgatgtggcagagaagttgcttgtggggagaccttgcttttaacaggcttctggaaaagctagggaaaagtggttgcccgctttccccctttccctccccttcaagcaccgcttgagatttgggctttattattaagagctgctataaaaacagcttgcttaaatgttaagagaagcccaggggtacttcaagcattccttcggatgcttcactccagaaagagggagttgaggcaagtcctcattactaataaaaatcaggtgaggctgacacttctcttgaccttaggataatagcgctttgttgtctctcctgccacaggaaggctccatggttgtcctactttaagccttcggtgcctttagtgaggggtacctgaaaaatcttaaaaaaaggcttagcgcccacctcacccctccacccccacgccaacacagtttgctcacatgccagttactccagcataaagctgaaatctattcaatactattgtcccataactgatctgactttgtatgtaaatacagaaaaagcttgttcacctgttttcctcattttgtccactggtgaattcaactggaagctccttctatagtctgaagaataccatctgaaagaactagtggttcccaatccccacatttaaaatggaatgtttggtttatttaaagtaaataggctattttttcttactgggtctggcttctctggcccttcgcatacgtgtgtctgctgagtgttcctgcatgtaagaattaagaccaagggaggggagagagaaacccacacataaacaatgcactaaagatcactgaactgtttaaacatttccacttgccagtttaatttcttgaagactgttgcttgtttggaatgtttcttgtcactgattttaaggttgcatctggaaaagactaaaggcttcagtcccctcccaccaccagaaatgaacaaaaagcattttacctaaaaatacaccagcaaaatgtactcagcttcaatcacaaatacgactgcttaaaactgcagaaatttcctcaacactcagcctttatcactcagctggattttttccttcaacaatcactactccaagcattggggaacacaacttttaatcatactccagtcgtttcacaatgcattctaatagcagcgggatcagaacagtactgcatttacttgccaacagaacagacagacctgaagtcaagacaactgcattctctgtgaagtctgtaaaaaaaaaaaaagctacaacaagtaagccccaccccctcctcccatccctccaaattccaggaaaaaaattttgagtatgctgataaactcactgcaaggtctcatacactcactagaacagaaggaagagccaagcactcatatgcaattcaaaatcctgaatggcttcatgaaggatgaaatgcctctgcaaaggaaaattctttaaaaccccacaggcaccccctcccttccctatataaggtcaatagtgtaaaacattgcctaccactctaagattgtaagcatttaaagttaacatgcaaactttggaaatgtgaaaaagtattactctaatcactgtcaatttatagacccctgactttctggaaataaaatgtgtaattaccttttactctgatcataatctcccacctgtctaagaggttatttattccttatttagagggcctctattgccatgtgcctggaattattatatgctcatcactttatgaagaataaaatttgtctttcctgccttaaagttacattcgttcttccgctcaaatcctgatctggtccattaaagagtgttcgcagacaaagtttctgaaagattagagaagaatcccccccaagattgccccaacactgaactacagacaaacactattttatttaaataaggagacagctttctaaaagtatacattctctaataaaaatagtttattattttgaatgatttaatggttttctacacaatttacatcacaacatgtaaattttagcagtaacatctgattctaacagcacatcatgctattcctttcatagagccttcagagattcaatgctaaacaaatttccttagttggcatcaaggcactgatcactttagaggcttttaagaaattatttaaagatgcaaatgcctctgagtgaagtgtactatcccatcactgaagcccacaggaacaagtcctacaattttaaaaaggctcgatggaaaaatttctcaatcctgaaatcccctagggaaggggtcaggagaaagtgccatggttgatatttaagaactccacagctcttaaaaataagcacttatccctaacatgcaatactgcagatgcaagttaaacttatctgttaacagctgcctgctgttttctgctcccagatgaaatgaagcaactcttctgataacgaagagatacctgtctgaggcaaacgaaacattggcacacagcacagcctcctcaatccacttgatcccaactcatctctcatttatttcggcttcttttattccaggattaatgtagtgtaacattttcatttcttttcgcttttattctgcttttgtaaaagcagtattttgagatggacattgcctcttcattgtatttctcatcaattcattatttttgtggttatagcttgacaagcaattaactttaaaatggtagattccgtaactttaaattggtagctttcatttgcttaaaattttttggcatatgcagataatgttctcatcagtagtaagaatctcagggttatgcttattccccaatggaggtatgacatataatcttttctgcctttacttatcaattcaccaaggagctgttttctctgcatctaggccatcatactgccaggctggttatgactcagaagatgttatctgaaaaaagtctatagaaaaaaaaaaagtttcccctccctcatcaacaaaagcccaccctctaagagacattcaagctgaactatcacaattcttaatcagttacaatttacaaacagataagtttaaaataaacaatttacaaaatttttgaagcataccttaacatcttgttttgcagttaaacaatggaaaagtatttctcctacactaaaaaaaaacttgcttacacacaactgaaaatagaatcttacttgataatacaaaagctaccatcagaagaaatcccttcaggatcattaagccacttcctttgctctgcagtttctatagtagttttaaattattattaaatcacctgaaaaaaattccaaaagagaaccacacactaccatatccaaacaacttttgcatttcccataattgtagttaatgtcagcccagtaggccagaccaacccccagttcaatactttccttccccaaaagctctatactttgaaggaaaacagatacagtatcaaattatgacactttccttgcccaaattaatgcactggtacacccagtggctcatatttaacttcccccagcttcccaattcaaactggggggaaaaaaactaaatcattgggagttacttgccaacttggaagttgatatttctttactttttccattctaagactttaagttctctggcatgagtttatctgcaatcataaactaaacaattacctaaacccaccccaccaatcccaaccgtaacaggccactgccaactaattgccaatatttgcccctcccctttaataaaacttttaagaagtcacattattggaaaacttaacttcaacatttggcctactcaagctcttctgaagttctcctgagatgactgaatatgaaccaaagctgcactgtgctgtacttttcagcttcaactgggaatactcttccaaggataaaagcagctccagtccctgaaggtgttcgtgccaacagcacagcggtacactccttctctaacccagtttgtcaatagtactatagcatctgtggaaaatcttagaaaaaaacattttctcccccaccctctctcttccctgttaagaccatcccaaaatgcttcaagtaaaaaataacaagtttaaggggttaagcacttttaaagtctgattaagggggtggggggaaaaaagagtaactaccagccatttctccaatggacatctcttccacagacctcaacgtgagaactgctctagtttctataaactgtaaacctgtggtggtctgattatcctgatattggattttcttgttttctgttacaccttgagtcatttgcctttaggattctagacagacctaagggaaaaagaactgaaaacatattttgcccccacccccacaaaaaaaaatactgaaaactcccccccgcctcagttacacatccaaactctacatttacaaaacgaattcagggtgaggaagtaaaaacaggtcatctattcacaaaactgaaatacttcattaccccaactaaacatacaaactgcttacagatttgctgaaattgtctcaatttggctatcaaattcatttggttttcctcaaattcgtgtaaaaaaaaaaaacccacaaacttgccatctactatttctccaggacttggcagtctgccctcaaaagcttcagacaagattcatgagtataagcctgaaaaagagaaacctacaacacccggaaatcggcctacgtccccatataaatccctttacacctcagtacgaaactcccccattatattagaaaataaataaaaggttaccataagtaagttccagaaaaacaacatattgccgacctcacggatttttaccaaccactcgctttccctgtggccttggcacactggcatgctggtctaggatcctctacgcacaacgcctgctcgcctcctccgtgtggttgccaagccaagccgcctgctaccttcatcaccaaattgcactcgctccttcctggaatcctttttcctagcttcaccaccaaatcgttagcgctccttccttcttcctagcttccttcaccaaatcgcactggctcctggactcttttcctatcttcaccacgaactgctgcttgctcgcttgctcctcagtcctagcttcatcaaacactggttcctggaatcctgtctgctgctgtcttcctagattcactgaatccacttctgtgtagcacctgggtcagctgtcaattaatgctagtcctcaggatttaaaaaataatcttaactcaaagtccaatgcaaaaacattaagttggtaattactcttgatcttgaattacttccgttacgaaagtccttcacatttttcaaactaagctactatatttaaggccttccaaattcttctaactcttccaaaagccttctgccttagttttttttaaattacaccagtccttttagtagctttttgatgtgatttttaaccaacttccccttctagcttcaagtattcttctaaattggttctggtctacgtaaacaccctcatcttctcaagctttaccttctaacttctgcaccaccagaaattaaattgatgggcttttaaaataaattggttaccaataatttcctcattttttcagtgctattttatccaatttttggctttatatttttctatcttctatacttctccaatacttgtcttagcttgtttttcattttctatctgaaactcttgacaatattttcattttctatcttgtttctatcttccaattttcttctaagtttgtacattttgcccttagctttttgtttcctagcttgtcttttttcttctgcttcctacttttcaggtttaaatttatcttttttcttctaaaagtatgtttttatcttctaatttccctatcttctctattcttttcttcgccttcccgtacttctgtcttccagttttccacttcaaacttctatcttctccaaattgtttcatcctaccactcccaattaatctttccattttcgtctgcgtttagtaaatgcgttaactaggctttaaatgacgcaattctccctgcgtcatggatttcaaggtcttttaatcaccttcggtttaatctctttttaaaagatcgccttcaaattattttaatcacctacaacttttaaactaaactttaagctgtttaagtcaccttcattttaatctaaaagcattgcccttctattggtattaattcggggctctgtagtcctttctctcaattttcttttaaatacattttttactccatgaagaagcttcat

SEQ ID NO: 109 - DNA, 4607 bp, Homo sapiens
  source 1..4607: mol_type = genomic DNA
gaatggagaagttgggtaacctgccccaggtgaagcccaaattggatctcagcgcctgggagctccaactttagcaagtgcgaaatcctgtcttcggttttggaattttcatccgcacagactatatcagccacggataggatatgtgagaataccagcatgtcaaggctgggaatcatccttaggcatcacctagccagtcctgcctctcattttaaggtgatcacaggatctggtttgccactgaaaggaatcttgatgctgaccacttcacctcggagagcatgaactgctcccatgccttcaagagttttaggatggcatcaactgttccaaaaacattgctgagctggactttgtatttcccagcatgagtcttacgttggacattcttcatgacaagatgagtatagtgctcagtgttttcaatagtaatgttttctgagttttgcaaaagtttctgaccatggaaccaagtcatggcaggtactggacgaccaatgtacataacatgaagccgaagtgtggaacccacagctccataatatttctctttcagtgggtaaccaggatggaactgcggtgttgcttgcaggagaagcttactactggtttctacttctccaacctcattggtggctatgcaggcactgtctccagactgtctatattcaacccagtatccaagaatttctttaccaccatcacattcaggtttctcccactgtagagtgacactatctttggatattgaaagaatctcaagttctcctggttggcttggtttatctgaaatattttaaaataatgaaaagggagtcagctttactggtgaaataaaaggaccaaacatggcttgcttctttaatttaaccccttcttctgaattccttaccaaatggatctttgcaaacaactggttcagaagcagggctggtctcactcaggccaacatcattctgtgcgatgatgcggaactgatactcagcatcgggaacaagccctgtgacagtgtacattgtggtggtgatctgagtcttgttgtgtctgacccacttgtcagtggatgtctctttgcgttcgatgtagtagcctgtgactctagaaccaccatcatctttgggccgggaccaggacaagctaacagaactcttggttacatcgagtacttctggaggattgcttggaggttctggaggatctgtaaatataagtggaaagcacacatgtattagaatacagtcccaagtattataagccaatgactttcatttaaaaacagaaaagtgctgaaataatgtttataattttgtggttgaaagggcacttactcaatggtgtttttggtgtgactggttcctcagatttcaagggtttgcttatgccaaactggttttctgctgaaacacggaaatggtattctacattctctttgaggccttttaccaccagagatgtacctcggactctggaatcaatggtataccaggcggctttaggcacttctcgtctctcgaggatgtagcctaagatgtcagcaccaccatcatcagcaggaggtctccagctgaccctcacagagcggacttggatgtcatcatattccagtggcccttctggactgttgggacttcctatcaccctgaccttgatgtagacagccttcttgccacatttattttccagaaccaggtcataagtgccagaatcacccctgtctgcttctttgatcacaagctcagtgtgtgtttcagatgttgcaatcatggcacgcttactaatatcctggccttccttggtccatttacatattgggaatggttttcctttgattggtatgcaggctcaccaggtccaccagcattacaagctaggacgcggaacctgtattctgcaccctgagaatcagaggtggggagagtggtggaagggcctgtggacttacggatgctgctgcgacactctatgacctcagactgcaagtaagagccaatcccgaagcggtttgttgcagccacacggaacacatactcatttccttcgatgctggaggacccggtttatctgaaggaataaggaagaagagtgaatatgtggaatggggaatgagtatagagtagaggtctggagacctgggctgcagtctttctattgtcaatttcctatatgacctaggagcagtaatggtatagtctcctgagtccttccggttcacgctgaatagctccaaggtgcacagattccgctgcatggccatctttatgccttctgctggttttagcagtgtgccatcttttttccaagaaactgttggcatcggtttaccaaaaacagtagcatccaagcagacattagttccagctttcacagtgtatatttagttttttattaggaaaaatgaccatcatgaatgctgttacatctttgcctataatactcagcagaatcttaccattttctgcgaggatagtcactggatcagaaggttcagaaggtgggctaatgtttaccgcggtcctggcaatagctctaaattgatactgagctttctcttctaggccagtagctgtgtactcttcctggggaatctggtgaggtgcagtattgcaccgtacccatttatcaccaggaagtttgcaagcttctacgaaatagccaataattttactgcctccatcatgctttggacgagcccagatcagagatacagtactcttggtgacatcaataacctcaggtttcccaggaggatctaaaacaaaaagaggaacacctggagatgaagacaaggaagatgtcagtttctacattaagtaacaaactagaaagaaaacaactttttttctccttcacaaaaacataccaaatgggaactgcgcaaccatctttggagatgtgagaggctctgaaatgccaaatcggttttcagcacggacccggaagatgtactcctggcctgggatcagctttccaaccctgcaggaagtttttgtgactgaagctagagccgtgacccagtcacctcggcttacatcacacttctccactatataattggtgatgttactgcctccgtcctccagagggatgtgccatgacagggagcaagcatcagcgtctatatcagaaatgtcaaatggaggctgaggggggccgggggcatctacatgaaccaagaggaaagaaatgtggctgccatgagatagtcatgtgttcaggagtaacatccagaatattaataggacctgttggtggcccaggaacatcaagaacagtaagatgtacggctactgttttgctaccggctgcattggaaactgtgatttgatattccccagtgtctctccttaagcagttcttaatgagaatagacgccttgatggctcctctggcagttcttgatgaccatggatgagctaatggctgtggtctcaatggtggcttcttgagttactttggatccaccatcttttagtgggggagaccactccagatctgcagatgatttagtccaatctgtcactttgggaaatggaggaccaggaggggctgcaaagagccagtatacattatttacagctgtgatggtatataagccagtgtcactcctgcgagactgcggaataactaaagtgcaagtatcatctaccaccagtttgttgacatgggtgtcatagagaacaggttctttgttatcaggcttctttggaggagctttaaaccagagaaataaaaacttgattgtgaagataaatgggtctcagcagttgctaagaaaatgactggttactttctgtcccttttgaggagcatgcagagaaattgctttagcctctgataaataaaacagactgcgaggaaaattacagcgaggaaattacagacatcatctcctttgaacagggaaaaggtaacagtgttggtgacttcctcccattctgctttcttcctacttgcatcacgtttgtcaataacataatgggtgatttcactgccaccattatcaagaggggcatcccatgtcaagtagcaagattcagctttaatgtcagtaacagcaagatttcttggtggggatgggcggtctggaaaggaatcaacagagaatattgagaaggcaattcttaaacagacactggatgcctttttgatgtaagaaagcaagtcacttaccatatacttcaacgtgaacatttcggaacactgagccaaggcgattggaagcagtaactgtgtaagtgcctttgtcctcccggaccgctttgggaatgctaagctcagtttttgcctcacttcgggatacctcttccttggttatctgaagtgcatcagtgggtttttcaattacagtttcatttttggaccattcaatcttaggcattggaaggcctgtcacatctgcagggatgtggacaggctctcctgccttaactttgatagtgtctcctcgaacactcagacgcaacttaatagttggaggcactgcaaagagaagagaaagaaaaacagtaacaaagtcataaggatgtttttttcaacttatgggataaaggcacactgtaaaatgcattaaaattattattatattcagaaaaaaaaaaaaaaaa

SEQ ID NO: 110 - DNA, 2314 bp, Homo sapiens
  source 1..2314: mol_type = genomic DNA
tgacattcttctggacaatgagtcccatcatctctccaccatgcaccttgtgactccctcctctgctgacaacagataaccacctttaactgtaactttccacagcctaccccagccctataaagctgcccctctcctatctcccttcgctgactctcttttcagactcagcccacttgcacccaagtgaattaacagccttgttgctcacacaaagcctgtttaggtggtcttctatacggacatgcttgacacttggtgccaaaatctgggccagggggactccttcgtgagaccggccccctgtcctggccctcattccgtgaagagatccacctgcgacctcgggtcctcagaccagcccaaggaacatctcaccaatttcaaatcggatctcctcggcttagtggctgaagactgatgctgcccgatcgcctcagaagccccttggaccatcacagatgccgagcttcgggtaactcttacggtggaggattcccagccatatgaagacaccctagctggacgatcagtccttgtcaaaagtctgacccctcaaactctacagcctcaatggaccagaccctacccggtcatttatagcacaccaactgccgtccatctgcaggaccctctccattgggttcaccattccagaataaagccatgcccatcagacagccagcttgatctctcctcttcctcctggaagccacaagattaggccgagagccgatcagacaaacaacctacaacccttaagctcctggcagcgcccagccaaggccatgcttccatgcaacactccttccaaatggccatcccagcatgcttccaagcaggcttcatccgttcctctggaccctcatctcttaagacctgccgcctataaaaaggattatatcttgagaccctatcctctaaaattttttccacacccaaaacaaaaaatctctgggtcaaaagtctaaaacgcttaggctggcaaccatcagatccttgcccatggtgtcctcaagcctactctcatgaaatggacaacagtacacgcatatggggccagttccacatatttggcaaccagaccagcatccaggacaacacaaagtatgttgtttgttgttagagggcttgggacatttcactctttgccagcctcagcttaatccaggagacaaagattattttccttattatctcttctgcataggatctgcaatcagaactattgaacttctccattcagaccgccactcacacctatgggaaaagggtaatgtatcatcggcttagcaacagggaatactattcgtatgatggaaaatggggacaaaaggctttggtacataaaacattattccttccttggcctaaaaactcatcgccacctacattaaagctaatatgcctgattactgtttttagagaacttattttattagggcagttccaagctcaaaaatacgctaactggcaccttgttagctacataaaaatgcaccctagacccgaaacttactagactcattataaaattttctttaaggtgtccacgcagtccctggtcacacttgaagcagtccggagaaatatcagccctaccccagtaatccccagaaggaacttacacttttttttaatcttttcctacaacttcatattttataaataaaaagacaaaaatgtcaggcctgtgagctgaagcttagccattgtaacccctgtgacctgcacatatccgtccaggtggcctgcaggagccaagaagtctggagcagccgaaaaaccacaaagaagtgaaacagccagttcctgccttaactaattaacccaccttacgacattccaccattatgacttgtccaccattatgacttgttcctgccctgccccaactgatcaatcaaccctgtgacattcttctcctggacaatgagtcccatcatctctccaccatgcaccttgtgaccccctcctctgctgaggataaccacctttaactgtaactttccacgcctacccaagccctataaagctgcccctctcctatctcccttcactgactctcttttcggactcagcccacttgcacccaagtgaattaacagccttgttgctcacacaaagcctgattgggtgtcttctatacggacacgcgtgacaggaacctcaacccaaaggcagtctgatgaggtgtctaagataaaagtagcggcacaaaggcttttgtaaacagaggcgtttcatgtggttttcctttcctttccttatatgtgaaaaggtgacagaaaagaaatcttcctaaaagagtcag

SEQ ID NO: 111 - DNA, 2685 bp, Homo sapiens
  source 1..2685: mol_type = genomic DNA
aagatggccgaataggaacagctccggtctacagctcccagcgtgagcgacgcagaagacgggtgatttctgcatttccatctgagttaggcttggccacaagagacaaattgcaaggcatggagacatttttctaacacttgaaaattctatgcaagagcaccaggcagctcagtcaccttgtcacttatctactgggtcaccttgttggcatgggatgccagccaggcttgcagctgctccatcctcccctgaatttgtacttcagcttctccactcatggcctgccctgaggacttcatggttcagcatcagatatgaagacgacagccctatagagactatttaaccagcttctaaggtggttaaatatcttaccagcctatgtaaggccgaatcctactaacaaagcactggtatatgtaatatcccagtggtttgcttctctaattgaaccctgactactacagaagtccagataccacagtctgagtagttctttaatctgcttatctggtgattccatcacaaatggttcagttgggcaaaaggctccagtgaagagctgtgaggcttatgtatggggactcaccaggggccagcccaggctgcccacaagaccatgagccaccctgtgactgaatgaagtggagagaagggaaagtagttaagaaatcaagaaaagtgtctttcagcaccatggccagaagttgtgccacagaattggaacagggaaacccagagagccagtgctgtcctagtcctagtgctgaatgatcccattggtgtccttagaagggctctgacttcagcccaccagcagcatgtggacttataagctttctagcagagttggagatccccaggaaaaagaaatctcttaaaatgaaatgaaacttcaatcaaccaaggctttctagcctgatgtgagttgtctttacacacatatacctttttaacaaagaagaagaaaagaatggacattgatagaacttgcattctgtgttcatcaccaggatagttgctttataaatatcattttgtttttagtcacaacccaatgcagcattttccttactgacaaagaacctgaggcatggagaatttaagcaactccctgaaggccacccagctaatcagcagtgggaaaagaatttaattctagatctttcattctaaagcccagctccttacacacgttggatgctactatttccaaaatgtcaacaacactttggactatcaacctgtctaaagagtatttgaagcagtgatgcaggcctctgaaggaaaaggtgcacattttcaacaataaaagagagacattcctttagtgtatctccacattccctgaaggggagctggaatgacatgacggacttggcctcagggccccaagccttccctgttgcagtcttcatgacactcctcagaagcagcaactccagccatgtctggaaccattccttaccagacaggaggatatcatggcctcaactcatgatctgtaccaagccaaacctcttttggggtacccaccaagcctcaggcctcaccttctctgccctgttgtccctgcccatctgtcctggttttctactagtttccactactggacacttgtctgaggctgggtccccacctggatcttgtggcctcttgcccagggcttggagggaagctgctcccacacttgaagatgacagctgagcatggtcctttgtgggcgtgctgtcaggaagatgacagtgcaatctgtttaatgtgccagttgtctctgtgagtccctcatccggatgagcacttgtcacacccctgcagagccccagctggttcttagagaggtgctccagctctgtgattacctaatctggcttcccaatctatcccacaatgtgtcattgctgaggtccctgtgggtcttgcagataaacttgctacagtcatcctacacaaaaaagaagctcgttatcatgaaaccggcctgctttttagctcctgggccctaactcaccccagattgctgccagctcttttgttttcccacttagaccaaagggattgagaagctttcccattccttggagctgccaccctccattctaaggcagtctcccccttcaccctgcttgctgcaccccagatgctgcaaggcccatgtgctgtttttgacttcagtacttcagattgctgtgggaacacaggaggcagcagccagatgagaaattgagtctgactctggagtattataaagtccttatagttactggcattaggtatagggtctgtattattaaagagaaattattcaccaaacacttgttaaaaatggcaagacagtttatttaagagcattgcaataggtaagtgctatggtctcaatgtttgtgtctccctcaaattcataagttgaaacttcacttccaagatgaaggaattaggaggtgggcactttaagggatgattatgtcataggccagagccctcatgaacgagatcagtgcccttctaaaagaggcattgggagagacccctcaccttttccatcatatgaggacacagccaggaagcatcatccacgaaccagaaaattggcccttaccagacactgaatctgctgatgtcctgaccatggacttctgagccttctgaactgtgagagataaatttctgttgtttataagc

SEQ ID NO: 112 - DNA, 200 bp, Homo sapiens
  source 1..200: mol_type = genomic DNA
ggccgggcgcggtggctcacgcctgtaatcccagctctcagggaggctaagaggcgggaggatagcttgagcccaggagttcgagacctgcctgggcaatatagcgagaccccgttctccagaaaaaggaaaaaaaaaaacaaaagacaaaaaaaaaataagcgtaacttccctcaaagcaacaaccccccccccccttt

SEQ ID NO: 113 - DNA, 634 bp, Homo sapiens
  source 1..634: mol_type = genomic DNA
cttttcgaggtaggagtcgactcctgtgaggtatggtgctgggtgcagatgcagtgtggctctggatagcaccttatggacagttgtgtccccaaggaaggatgagaatagctactgaagtcctaaagagcaagcctaactcaagccattggcacacaggcattagacagaaagctggaagttgaaatggtggagtccaacttgcctggaccagcttaatggttctgctcctggtaacgtttttatccatggatgacttgcttgggtaaggacatgaagacagttcctgtcataccttttaaaggtatggagagtcggcttgactacactgtgtggagcaagttttaaagaagcaaaggactcagaattcatgattgaagaaatgcaggcagacctgttatcctaaactagggtttttaatgaccacaacaagcaagcatgcagcttactgcttgaaagggtcttgcctcacccaagctagagtgcagtggcctttgaagcttactacagcctcaaacttctgggctcaagtgatcctcagcctcccagtggtctttgtagactgcctgatggagtctcatggcacaagaagattaaaacagtgtctccaattttaataaatttttgcaatcca

SEQ ID NO: 114 - DNA, 2513 bp, Homo sapiens
  source 1..2513: mol_type = genomic DNA
gcgcgcagatcgctccggacccggacaccgcctgcgaggagcgccgaccagccgggaagggttcgcgctaggcggcgcccgggtcccgtcggccagggtgagcgtccggcccgcgtccgcgccacgcccgccgcgttcccctttcctctgcggcgggccgagagataaccctgcccgaggggtcccggcgcccgccccccacgcggtcgcactggaattcgcagcccctctcgggtccccggggcgcattttgcagtctgagtggcaatgcacttgctccaggacaggcggctaccccgccgcagcggaggcgcggacttttcttttggggggtctcgccggctcgccgcgctccccaccttgcctgcgcccgcccggagccagcggttctccaagcacccagcatcctgctagacgcgccgcgcaccgacggaggggacatgggcagagcaatggtggccaggctcgggctggggctgctgctgctggcactgctcctacccacgcagatttattccagtgaaacaacaactggaacttcaagtaactcctcccagagtacttccaactctgggttggccccaaatccaactaatgccaccaccaaggcggctggtggtgccctgcagtcaacagccagtctcttcgtggtctcactctctcttctgcatctctactcttaagagactcaggccaagaaacgtcttctaaatttccccatcttctaaacccaatccaaatggcgtctggaagtccaatgtggcaaggaaaaacaggtcttcatcgaatctactaattccacaccttttattgacacagaaaatgttgagaatcccaaatttgattgatttgaagaacatgtgagaggtttgactagatgatggatgccaatattaaatctgctggagtttcatgtacaagatgaaggagaggcaacatccaaaatagttaagacatgatttccttgaatgtggcttgagaaatatggacacttaatactaccttgaaaataagaatagaaataaaggatgggattgtggaatggagattcagttttcatttggttcattaattctataaggccataaaacaggtaatataaaaagcttccatgattctatttatatgtacatgagaaggaacttccaggtgttactgtaattcctcaacgtattgtttcgacagcactaatttaatgccgatatactctagatgaagttttacattgttgagctattgctgttctcttgggaactgaactcactttcctcctgaggctttggatttgacattgcatttgaccttttatgtagtaattgacatgtgccagggcaatgatgaatgagaatctacccccagatccaagcatcctgagcaactcttgattatccatattgagtcaaatggtaggcatttcctatcacctgtttccattcaacaagagcactacattcatttagctaaacggattccaaagagtagaattgcattgaccacgactaatttcaaaatgctttttattattattattttttagacagtctcactttgtcgcccaggccggagtgcagtggtgcgatctcagatcagtgtaccatttgcctcccgggctcaagcgattctcctgcctcagcctcccaagtagctgggattacaggcacctgccaccatgcccggctaatttttgtaattttagtagagacagggtttcaccatgttgcccaggctggtttcgaactcctgacctcaggtgatccacccgcctcggcctcccaaagtgctgggattacaggcttgagcccccgcgcccagccatcaaaatgctttttatttctgcatatgttgaatactttttacaatttaaaaaaatgatctgttttgaaggcaaaattgcaaatcttgaaattaagaaggcaaaaatgtaaaggagtcaaaactataaatcaagtatttgggaagtgaagactggaagctaatttgcattaaattcacaaacttttatactctttctgtatatacattttttttctttaaaaaacaactatggatcagaatagccacatttagaacactttttgttatcagtcaatatttttagatagttagaacctggtcctaagcctaaaagtgggcttgattctgcagtaaatcttttacaactgcctcgacacacataaacctttttaaaaatagacactccccgaagtcttttgttcgcatggtcacacactgatgcttagatgttccagtaatctaatatggccacagtagtcttgatgaccaaagtcctttttttccatctttagaaaactacatgggaacaaacagatcgaacagttttgaagctactgtgtgtgtgaatgaacactcttgctttattccagaatgctgtacatctattttggattgtatattgtgtttgtgtatttacgctttgattcatagtaacttcttatggaattgatttgcattgaacacaaactgtaaataaaaagaaatggctgaaagagcaa

SEQ ID NO: 115 - DNA, 5695 bp, Homo sapiens
  source 1..5695: mol_type = genomic DNA
aaccgacgcgcgtctgtggagaagcggcttggtcgggggtggtctcgtggggtcctgcctgtttagtcgctttcagggttcttgagccccttcacgaccgtcaccatggaagtgtcaccattgcagcctgtaaatgaaaatatgcaagtcaacaaaataaagaaaaatgaagatgctaagaaaagactgtctgttgaaagaatctatcaaaagaaaacacaattggaacatattttgctccgcccagacacctacattggttctgtggaattagtgacccagcaaatgtgggtttacgatgaagatgttggcattaactatagggaagtcacttttgttcctggtttgtacaaaatctttgatgagattctagttaatgctgcggacaacaaacaaagggacccaaaaatgtcttgtattagagtcacaattgatccggaaaacaatttaattagtatatggaataatggaaaaggtattcctgttgttgaacacaaagttgaaaagatgtatgtcccagctctcatatttggacagctcctaacttctagtaactatgatgatgatgaaaagaaagtgacaggtggtcgaaatggctatggagccaaattgtgtaacatattcagtaccaaatttactgtggaaacagccagtagagaatacaagaaaatgttcaaacagacatggatggataatatgggaagagctggtgagatggaactcaagcccttcaatggagaagattatacatgtatcacctttcagcctgatttgtctaagtttaaaatgcaaagcctggacaaagatattgttgcactaatggtcagaagagcatatgatattgctggatccaccaaagatgtcaaagtctttcttaatggaaataaactgccagtaaaaggatttcgtagttatgtggacatgtatttgaaggacaagttggatgaaactggtaactccttgaaagtaatacatgaacaagtaaaccacaggtgggaagtgtgtttaactatgagtgaaaaaggctttcagcaaattagctttgtcaacagcattgctacatccaagggtggcagacatgttgattatgtagctgatcagattgtgactaaacttgttgatgttgtgaagaagaagaacaagggtggtgttgcagtaaaagcacatcaggtgaaaaatcacatgtggatttttgtaaatgccttaattgaaaacccaacctttgactctcagacaaaagaaaacatgactttacaacccaagagctttggatcaacatgccaattgagtgaaaaatttatcaaagctgccattggctgtggtattgtagaaagcatactaaactgggtgaagtttaaggcccaagtccagttaaacaagaagtgttcagctgtaaaacataatagaatcaagggaattcccaaactcgatgatgccaatgatgcagggggccgaaactccactgagtgtacgcttatcctgactgagggagattcagccaaaactttggctgtttcaggccttggtgtggttgggagagacaaatatggggttttccctcttagaggaaaaatactcaatgttcgagaagcttctcataagcagatcatggaaaatgctgagattaacaatatcatcaagattgtgggtcttcagtacaagaaaaactatgaagatgaagattcattgaagacgcttcgttatgggaagataatgattatgacagatcaggaccaagatggttcccacatcaaaggcttgctgattaattttatccatcacaactggccctctcttctgcgacatcgttttctggaggaatttatcactcccattgtaaaggtatctaaaaacaagcaagaaatggcattttacagccttcctgaatttgaagagtggaagagttctactccaaatcataaaaaatggaaagtcaaatattacaaaggtttgggcaccagcacatcaaaggaagctaaagaatactttgcagatatgaaaagacatcgtatccagttcaaatattctggtcctgaagatgatgctgctatcagcctggcctttagcaaaaaacagatagatgatcgaaaggaatggttaactaatttcatggaggatagaagacaacgaaagttacttgggcttcctgaggattacttgtatggacaaactaccacatatctgacatataatgacttcatcaacaaggaacttatcttgttctcaaattctgataacgagagatctatcccttctatggtggatggtttgaaaccaggtcagagaaaggttttgtttacttgcttcaaacggaatgacaagcgagaagtaaaggttgcccaattagctggatcagtggctgaaatgtcttcttatcatcatggtgagatgtcactaatgatgaccattatcaatttggctcagaattttgtgggtagcaataatctaaacctcttgcagcccattggtcagtttggtaccaggctacatggtggcaaggattctgctagtccacgatacatctttacaatgctcagctctttggctcgattgttatttccaccaaaagatgatcacacgttgaagtttttatatgatgacaaccagcgtgttgagcctgaatggtacattcctattattcccatggtgctgataaatggtgctgaaggaatcggtactgggtggtcctgcaaaatccccaactttgatgtgcgtgaaattgtaaataacatcaggcgtttgatggatggagaagaacctttgccaatgcttccaagttacaagaacttcaagggtactattgaagaactggctccaaatcaatatgtgattagtggtgaagtagctattcttaattctacaaccattgaaatctcagagcttcccgtcagaacatggacccagacatacaaagaacaagttctagaacccatgttgaatggcaccgagaagacacctcctctcataacagactatagggaataccatacagataccactgtgaaatttgttgtgaagatgactgaagaaaaactggcagaggcagagagagttggactacacaaagtcttcaaactccaaactagtctcacatgcaactctatggtgctttttgaccacgtaggctgtttaaagaaatatgacacggtgttggatattctaagagacttttttgaactcagacttaaatattatggattaagaaaagaatggctcctaggaatgcttggtgctgaatctgctaaactgaataatcaggctcgctttatcttagagaaaatagatggcaaaataatcattgaaaataagcctaagaaagaattaattaaagttctgattcagaggggatatgattcggatcctgtgaaggcctggaaagaagcccagcaaaaggttccagatgaagaagaaaatgaagagagtgacaacgaaaaggaaactgaaaagagtgactccgtaacagattctggaccaaccttcaactatcttcttgatatgcccctttggtatttaaccaaggaaaagaaagatgaactctgcaggctaagaaatgaaaaagaacaagagctggacacattaaaaagaaagagtccatcagatttgtggaaagaagacttggctacatttattgaagaattggaggctgttgaagccaaggaaaaacaagatgaacaagtcggacttcctgggaaaggggggaaggccaaggggaaaaaaacacaaatggctgaagttttgccttctccgcgtggtcaaagagtcattccacgaataaccatagaaatgaaagcagaggcagaaaagaaaaataaaaagaaaattaagaatgaaaatactgaaggaagccctcaagaagatggtgtggaactagaaggcctaaaacaaagattagaaaagaaacagaaaagagaaccaggtacaaagacaaagaaacaaactacattggcatttaagccaatcaaaaaaggaaagaagagaaatccctggtctgattcagaatcagataggagcagtgacgaaagtaattttgatgtccctccacgagaaacagagccacggagagcagcaacaaaaacaaaattcacaatggatttggattcagatgaagatttctcagattttgatgaaaaaactgatgatgaagattttgtcccatcagatgctagtccacctaagaccaaaacttccccaaaacttagtaacaaagaactgaaaccacagaaaagtgtcgtgtcagaccttgaagctgatgatgttaagggcagtgtaccactgtcttcaagccctcctgctacacatttcccagatgaaactgaaattacaaacccagttcctaaaaagaatgtgacagtgaagaagacagcagcaaaaagtcagtcttccacctccactaccggtgccaaaaaaagggctgccccaaaaggaactaaaagggatccagctttgaattctggtgtctctcaaaagcctgatcctgccaaaaccaagaatcgccgcaaaaggaagccatccacttctgatgattctgactctaattttgagaaaattgtttcgaaagcagtcacaagcaagaaatccaagggggagagtgatgacttccatatggactttgactcagctgtggctcctcgggcaaaatctgtacgggcaaagaaacctataaagtacctggaagagtcagatgaagatgatctgttttaaaatgtgaggcgattattttaagtaattatcttaccaagcccaagactggttttaaagttacctgaagctcttaacttcctcccctctgaatttagtttggggaaggtgtttttagtacaagacatcaaagtgaagtaaagcccaagtgttctttagctttttataatactgtctaaatagtgaccatctcatgggcattgttttcttctctgctttgtctgtgttttgagtctgctttcttttgtctttaaaacctgatttttaagttcttctgaactgtagaaatagctatctgatcacttcagcgtaaagcagtgtgtttattaaccatccactaagctaaaactagagcagtttgatttaaaagtgtcactcttcctccttttctactttcagtagatatgagatagagcataattatctgttttatcttagttttatacataatttaccatcagatagaactttatggttctagtacagatactctactacactcagcctcttatgtgccaagtttttctttaagcaatgagaaattgctcatgttcttcatcttctcaaatcatcagaggccgaagaaaaacactttggctgtgtctataacttgacacagtcaatagaatgaagaaaattagagtagttatgtgattatttcagctcttgacctgtcccctctggctgcctctgagtctgaatctcccaaagagagaaaccaatttctaagaggactggattgcagaagactcggggacaacatttgatccaagatcttaaatgttatattgataaccatgctcagcaatgagctattagattcattttgggaaatctccataatttcaatttgtaaactttgttaagacctgtctacattgttatatgtgtgtgacttgagtaatgttatcaacgtttttgtaaatatttactatgtttttctattagctaaattccaacaattttgtactttaataaaatgttctaaacattgcaa

SEQ ID NO: 116 - DNA, 6013 bp, Homo sapiens
  source 1..6013: mol_type = genomic DNA
aacggcgggagattcaaacctggaagaaggaggaacatggagaggagagcagcgggcccaggctgggcagcctatgaacgcctcacagctgaggagatggatgagcagaggcggcagaatgttgcctatcagtacctgtgccggctggaggaggccaagcgctggatggaggcctgcctgaaggaggagcttccttccccggtggagctggaggagagccttcggaatggagtgctgctggccaagctaggccactgttttgcaccctccgtggttcccttgaagaagatctacgatgtggagcagctgcggtaccaggcaactggcttacatttccgtcacacagacaacatcaacttttggctatctgcaatagcccacatcggtctgccttcgaccttcttcccagagaccacggacatctatgacaaaaagaacatgccccgggtagtctactgcatccatgctctcagtctcttcctcttccggctgggattggcccctcagatacatgatctatacgggaaagtgaaattcacagctgaggaactcagcaacatggcgtccgaactggccaaatatggcctccagctgcctgccttcagcaagatcgggggcatcttggccaatgagctctcggtggatgaggctgcagtccatgcagctgttcttgccatcaatgaagcagtggagcgaggggtggtggaggacaccctggctgccttgcagaatcccagtgctcttctggagaatctccgagagcctctggcagccgtctaccaagagatgctggcccaggccaagatggagaaggcagccaatgccaggaaccatgatgacagagaaagccaggacatctatgaccactacctaactcaggctgaaatccagggcaatatcaaccatgtcaacgtccatggggctctagaagttgttgatgatgccctggaaagacagagccctgaagccttgctcaaggcccttcaagaccctgccctggccctgcgaggggtgaggagagactttgctgactggtacctggagcagctgaactcagacagagagcagaaggcacaggagctgggcctggtggagcttctggaaaaggaggaagtccaggctggtgtggctgcagccaacacaaagggtgatcaggaacaagccatgctccacgctgtgcagcggatcaacaaagccatccggaggagagtggcggctgacactgtgaaggagctgatgtgccctgaggcccagctgcctccagtgtaccctgttgcatcgtctatgtaccagctggagctggcagtgctccagcagcagcagggggagcttggccaggaggagctcttcgtggctgtggagatgctctcagctgtggtcctgattaaccgggccctggaggcccgggatgccagtggcttctggagcagcctggtgaaccctgccacaggcctggctgaggtggaaggagaaaatgcccagcgttacttcgatgccctgctgaaattgcgacaggagcgtgggatgggtgaggacttcctgagctggaatgacctgcaggccaccgtgagccaggtcaatgcacagacccaggaagagactgaccgggtccttgcagtcagcctcatcaatgaggctctggacaaaggcagccctgagaagactctgtctgccctactgcttcctgcagctggcctagatgatgtcagcctccctgtcgcccctcggtaccatctcctccttgtggcagccaaaaggcagaaggcccaggtgacaggggatcctggagctgtgctgtggcttgaggagatccgccagggagtggtcagagccaaccaggacactaatacagctcagagaatggctcttggtgtggctgccatcaatcaagccatcaaggagggcaaggcagcccagactgagcgggtgttgaggaaccccgcagtggcccttcgaggggtagttcccgactgtgccaacggctaccagcgagccctggaaagtgccatggcaaagaaacagcgtccagcagacacagctttctgggttcaacatgacatgaaggatggcactgcctactacttccatctgcagaccttccaggggatctgggagcaacctcctggctgccccctcaacacctctcacctgacccgggaggagatccagtcagctgtcaccaaggtcactgctgcctatgaccgccaacagctctggaaagccaacgtcggctttgttatccagctccaggcccgcctccgtggcttcctagttcggcagaagtttgctgagcattcccactttctgaggacctggctcccagcagtcatcaagatccaggctcattggcggggttataggcagcggaagatttacctggagtggttgcagtattttaaagcaaacctggatgccataatcaagatccaggcctgggcccggatgtgggcagctcggaggcaatacctgaggcgtctgcactacttccagaagaatgttaactccattgtgaagatccaggcatttttccgagccaggaaagcccaagatgactacaggatattagtgcatgcaccccaccctcctctcagtgtggtacgcagatttgcccatctcttgaatcaaagccagcaagacttcttggctgaggcagagctgctgaagctccaggaagaggtagttaggaagatccgatccaatcagcagctggagcaggacctcaacatcatggacatcaagattggcctgctggtgaagaaccggatcactctgcaggaagtggtctcccactgcaagaagctgaccaagaggaataaggaacagctgtcagatatgatggttctggacaagcagaagggtttaaagtcgctgagcaaagagaaacggcagaaactagaagcataccaacacctcttctacctgctccagactcagcccatctacctggccaagctgatctttcagatgccacagaacaaaaccaccaagttcatggaggcagtgattttcagcctgtacaactatgcctccagccgccgagaggcctatctcctgctccagctgttcaagacagcactccaggaggaaatcaagtcaaaggtggagcagccccaggacgtggtgacaggcaacccaacagtggtgaggctggtggtgagattctaccgtaatgggcggggacagagtgccctgcaggagattctgggcaaggttatccaggatgtgctagaagacaaagtgctcagcgtccacacagaccctgtccacctctataagaactggatcaaccagactgaggcccagacagggcagcgcagccatctcccatatgatgtcaccccggagcaggccttgagccaccccgaggtccagagacgactggacatcgccctacgcaacctcctcgccatgactgataagttccttttagccatcacctcatctgtggaccaaattccgtatgggatgcgatatgtggccaaagtcctgaaggcaactctggcagagaaattccctgacgccacagacagcgaggtctataaggtggtcgggaacctcctgtactaccgcttcctgaacccagctgtggtggctcctgacgccttcgacattgtggccatggcagctggtggagccctggctgccccccagcgccatgccctgggggctgtggctcagctcctacagcacgctgcggctggcaaggccttctctgggcagagccagcacctacgggtcctgaatgactatctggaggaaacacacctcaagttcaggaagttcatccatagagcctgccaggtgccagagccagaggagcgttttgcagtggacgagtactcagacatggtggctgtggccaaacccatggtgtacatcaccgtgggggagctggtcaacacgcacaggctgttgctggagcaccaggactgcattgcccctgatcaccaagaccccctgcatgagctcctggaggatcttggggagctgcccaccatccctgaccttattggtgagagcatcgctgcagatgggcacacggacctgagcaagctagaagtgtccctgacgctgaccaacaagtttgaaggactagaggcagatgctgatgactccaacacccgtagcctgcttctgagcaccaagcagctgttggccgatatcatacagttccatcctggggacaccctcaaggagatcctgtccctctcggcttccagagagcaagaagcagcccacaagcagctgatgagccgacgccaggcctgtacagcccagacaccggagccactgcgacgacaccgctcactgacagctcactccctcctgccactggcagagaagcagcggcgcgtcctgcggaacctacgccgacttgaagccctggggttggtcagcgccagaaatggctaccaggggctagtggacgagctggccaaggacatccgcaaccagcacagacacaggcacaggcggaaggcagagctggtgaagctgcaggccacattacagggcctgagcactaagaccaccttctatgaggagcagggtgactactacagccagtacatccgggcctgcctggaccacctggcccccgactccaagagttctgggaaggggaagaagcagccttctcttcattacactgctgctcagctcctggaaaagggtgtcttggtggaaattgaagatcttcccgcctctcacttcagaaacgtcatctttgacatcacgccgggagatgaggcaggaaagtttgaagtaaatgccaagttcctgggtgtggacatggagcgatttcagcttcactatcaggatctcctgcagctccagtatgagggtgtggctgtcatgaaactcttcaacaaggccaaagtcaatgtcaaccttctcatcttcctcctcaacaagaagtttttgcggaagtgacagaggcaaagggtgctacccaagcccctcttacctctctggatgctttctttaacactaactcaccactgtgcttccctgcagacacctagagctcaggactgggcaaggcccagggattctcaccccttccccagctgggaggagcttgcctgcctggccacagacagtgtatcttctaattggctaaagtgggccttgcccagagtccagctgtgtggcttttatcatgcatgacaaacccctggctttcctgccagatggtaggacatggaccttgacctgggaaagccattactcttgtgtctgctactgccctcccacagtcaccccaatattacaagcactgccccagcggcttgatttcccctctgccttccttctctctgcactcccacaaagccagggccaggctccccatccctacctcccactgcatcagcagtgggtgttcctgcccttcctgagtctaggcagctctgctgctgtgatctgcacaccctccaacctgggcagggactggggggatgcagtgtgtgttagtgcccatgtggcattgtggcactgttgccccccatggcggcatgggcaagatgaccttccattagcttcaagtcttgttctcttgtctgtggtctgtttaatatgtgggtcactagggtatttattctttctcccatccttacactctggatcattgtgcagacttaatcagggttttaacgctttcatttttttttttttttttttttttttgagctcaaagagagttctcattttccctattcaaactaatacccatgccgtgttttttaccttggatttaaagtcaccttaggttggggcaacagattctcactcatgtttaagatcttgttatttcagcttcataagatcaaagaggagtctttcccttttctcttttaccctcaggattctcatcccttacagctgactcttccaggcaatttccatagatctgcagtcctgcctctgccacagtctctctgttgtccccacatctacccaacttcctgtactgttgcccttctgatgttaataaaagcagctgttactccca

SEQ ID NO: 117 - DNA, 777 bp, Homo sapiens
  source 1..777: mol_type = genomic DNA
gcagttgcagtcgtgttctccgagttcctgtctctctgccaacgccgcccggatggcttcccaaaaccgcgacccagccgccactagcgtcgccgccgcccgtaaaggagctgagccgagcgggggcgccgcccggggtccggtgggcaaaaggctacagcaggagctgatgaccctcatgatgtctggcgataaagggatttctgccttccctgaatcagacaaccttttcaaatgggtagggaccatccatggagcagctggaacagtatatgaagacctgaggtataagctctcgctagagttccccagtggctacccttacaatgcgcccacagtgaagttcctcacgccctgctatcaccccaacgtggacacccagggtaacatatgcctggacatcctgaaggaaaagtggtctgccctgtatgatgtcaggaccattctgctctccatccagagccttctaggagaacccaacattgatagtcccttgaacacacatgctgccgagctctggaaaaaccccacagcttttaagaagtacctgcaagaaacctactcaaagcaggtcaccagccaggagccctgacccaggctgcccagcctgtccttgtgtcgtctttttaatttttccttagatggtctgtcctttttgtgatttctgtataggactctttatcttgagctgtggtatttttgttttgtttttgtcttttaaattaagcctcggttgagcccttgtatattaaataaatgcatttttgtcctttttta

SEQ ID NO: 118 - DNA, 6239 bp, Homo sapiens; identical to SEQ ID NO: 90
  source 1..6239: mol_type = genomic DNA

SEQ ID NO: 119 - DNA, 2574 bp, Homo sapiens
  source 1..2574: mol_type = genomic DNA
gcggcgcgccattaaccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcggagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgtttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttcaaacactgctgtggaccctactgggtttttaaaatattgtcagtttttcatcgtcgtccctagcctgccaacagccatctgcccagacagccgcagtgaggatgagcgtcctggcagagacgcagttgtctctgggcgcttgccagagccacgaaccccagacctgtttgtatcatccgggctccttccgggcagaaacaactgaaaatgcacttcagacccacttatttctgccacatctgagtcggcctgagatagacttttccctctaaactgggagaatatcacagtggtttttgttagcagaaaatgcactccagcctctgtactcatctaagctgcttatttttgatatttgtgtcagtctgtaaatggatacttcactttaataactgttgcttagtaattggctttgtagagaagctggaaaaaaatggttttgtcttcaactcctttgcatgccaggcggtgatgtggatctcggcttctgtgagcctgtgctgtgggcagggctgagctggagccgcccctctcagcccgcctgccacggcctttccttaaaggccatccttaaaaccagaccctcatggctaccagcacctgaaagcttcctcgacatctgttaataaagccgtaggcccttgtctaagtgcaaccgcctagactttctttcagatacatgtccacatgtccatttttcaggttctctaagttggagtggagtctgggaagggttgtgaatgaggcttctgggctatgggtgaggttccaatggcaggttagagcccctcgggccaactgccatcctggaaagtagagacagcagtgcccgctgcccagaagagaccagcaagccaaactggagcccccattgcaggctgtcgccatgtggaaagagtaactcacaattgccaataaagtctcatgtggttttatcta

SEQ ID NO: 120 - DNA, 81 bp, Homo sapiens
  source 1..81: mol_type = genomic DNA
cttctggaagctggtttcacatggtggcttagatttttccatctttgtatctagcaccatttgaaatcagtgttttaggag

SEQ ID NO: 121 - DNA, 71 bp, Homo sapiens
  source 1..71: mol_type = genomic DNA
ggagaggaggcaagatgctggcatagctgttgaactgggaacctgctatgccaacatattgccatctttcc

SEQ ID NO: 122 - DNA, 95 bp, Homo sapiens
  source 1..95: mol_type = genomic DNA
cggccggccctgggtccatcttccagtacagtgttggatggtctaattgtgaagctcctaacactgtctggtaaagatggctcccgggtgggttc

SEQ ID NO: 123 - DNA, 84 bp, Homo sapiens
  source 1..84: mol_type = genomic DNA
gtgctcggtttgtaggcagtgtcattagctgattgtactgtggtggttacaatcactaactccactgccatcaaaacaaggcac

SEQ ID NO: 124 - DNA, 110 bp, Homo sapiens
  source 1..110: mol_type = genomic DNA
ccagaggttgtaacgttgtctatatataccctgtagaaccgaatttgtgtggtatccgtatagtcacagattcgattctaggggaatatatggtcgatgcaaaaacttca

SEQ ID NO: 125 - DNA, 78 bp, Homo sapiens
  source 1..78: mol_type = genomic DNA
tactgccctcggcttctttacagtgctgccttgttgcatatggatcaagcagcattgtacagggctatgaaggcattg

SEQ ID NO: 126 - DNA, 78 bp, Homo sapiens
  source 1..78: mol_type = genomic DNA
ttgtgctttcagcttctttacagtgctgccttgtagcattcaggtcaagcagcattgtacagggctatgaaagaacca

SEQ ID NO: 127 - DNA, 62 bp, Homo sapiens
  source 1..62: mol_type = genomic DNA
tcatagccctgtacaatgctgcttgatccatatgcaacaaggcagcactgtaaagaagccga

SEQ ID NO: 128 - DNA, 88 bp, Homo sapiens
  source 1..88: mol_type = genomic DNA
caccttgtcctcacggtccagttttcccaggaatcccttagatgctaagatggggattcctggaaatactgttcttgaggtcatggtt

SEQ ID NO: 129 - DNA, 110 bp, Homo sapiens
  source 1..110: mol_type = genomic DNA
gagctgcttgcctccccccgtttttggcaatggtagaactcacactggtgaggtaacaggatccggtggttctagacttgccaactatggggcgaggactcagccggcac

SEQ ID NO: 130 - DNA, 940 bp, Homo sapiens
  source 1..940: mol_type = genomic DNA
cttgaaatgggctgagtccctcttgctcacccttgacttggaaaaaccagtttctcttttattgtctgttactaatctctattctaaaaattcagctcaattctcaaccatactccaaactctctcttttccagctacctttactccctctccttcaattccactttcctctgcttactttttttttttttctgacagggtctcactttgtcgcccgggcaggagtgcagtggctcaatcttgggctcactgcagcctcaacctcccagaggcggggtttcaccatgttgcccagactggtcttgaactcctgagcttaagcaatccacctgcctcggcctcccaaagtgttgggatcacaggcgtgagccaccgcatccggcctcatgttctttttcattaaagagagaaatcaactattcaggaccggcccccacctttcctcaggagtcatttctgttccgcacaggcctgctgaactgggtgctttatataggaaaggaccaaccacaccgagctcagttatggcacacacagtgggacctagacaaagggagagggtgaccgacatcccaactagatttcagtggagtgaagttcaggaggcatggagctgacaaccatgaggcctcggcagccaccgccaccaccgccgccgccaccaccgtagcagcagcagcagcagcagcagcagcagcagcagcagcaagagtaactctgacttaggaatagagacagccagagagaaatgtgatcaatgaaggagacatctggagtgtgcgtgcttcttcagagggacgggtgatgggcagattggaaaaagcaccgcagatgggaaccttaatctttcttttctaaaattgatgctatgaaaatttgcgttttctgtaacttgtaaaaactaaaagttgcttgtctactgaaaaaaaaaaaaaaaaaa

SEQ ID NO: 131 - DNA, 71 bp, Homo sapiens
  source 1..71: mol_type = genomic DNA
gtagcactaaagtgcttatagtgcaggtagtgtttagttatctactgcattatgagcacttaaagtactgc

SEQ ID NO: 132 - DNA, 75 bp, Homo sapiens
  source 1..75: mol_type = genomic DNA
tcatccctgggtggggatttgttgcattacttgtgttctatataaagtattgcacttgtcccggcctgtggaaga

SEQ ID NO: 133 - DNA, 84 bp, Homo sapiens
  source 1..84: mol_type = genomic DNA
gtcagaataatgtcaaagtgcttacagtgcaggtagtgatatgtgcatctactgcagtgaaggcacttgtagcattatggtgac

SEQ ID NO: 134 - DNA, 7472 bp, Homo sapiens
  source 1..7472: mol_type = genomic DNA
aggcattgaggcagccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagagaagatagggaaattagaagataaaaacatacttttagaagaaaaaagataaatttaaacctgaaaagtaggaagcagaagaaaaaagacaagctaggaaacaaaaagctaagggcaaaatgtacaaacttagaagaaaattggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagttggttaaaaatcacatcaaaaagctactaaaaggactggtgtaatttaaaaaaaactaaggcagaaggcttttggaagagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaaatgactcaaggtgtaacagaaaacaagaaaatccaatatcaggataatcagaccaccacaggtttacagtttatagaaactagagcagttctcacgttgaggtctgtggaagagatgtccattggagaaatggctggtagttactcttttttccccccacccccttaatcagactttaaaagtgcttaaccccttaaacttgttattttttacttgaagcattttgggatggtcttaacagggaagagagagggtgggggagaaaatgtttttttctaagattttccacagatgctatagtactattgacaaactgggttagagaaggagtgtaccgctgtgctgttggcacgaacaccttcagggactggagctgcttttatccttggaagagtattcccagttgaagctgaaaagtacagcacagtgcagctttggttcatattcagtcatctcaggagaacttcagaagagcttgagtaggccaaatgttgaagttaagttttccaataatgtgacttcttaaaagttttattaaaggggaggggcaaatattggcaattagttggcagtggcctgttacggttgggattggtggggtgggtttaggtaattgtttagtttatgattgcagataaactcatgccagagaacttaaagtcttagaatggaaaaagtaaagaaatatcaacttccaagttggcaagtaactcccaatgatttagtttttttccccccagtttgaattgggaagctgggggaagttaaatatgagccactgggtgtaccagtgcattaatttgggcaaggaaagtgtcataatttgatactgtatctgttttccttcaaagtatagagcttttggggaaggaaagtattgaactgggggttggtctggcctactgggctgacattaactacaattatgggaaatgcaaaagttgtttggatatggtagtgtgtggttctcttttggaatttttttcaggtgatttaataataatttaaaactactatagaaactgcagagcaaaggaagtggcttaatgatcctgaagggatttcttctgatggtagcttttgtattatcaagtaagattctattttcagttgtgtgtaagcaagtttttttttagtgtaggagaaatacttttccattgtttaactgcaaaacaagatgttaaggtatgcttcaaaaattttgtaaattgtttattttaaacttatctgtttgtaaattgtaactgattaagaattgtgatagttcagcttgaatgtctcttagagggtgggcttttgttgatgagggaggggaaactttttttttttctatagacttttttcagataacatcttctgagtcataaccagcctggcagtatgatggcctagatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtggattgaggaggctgtgctgtgtgccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaaggaaatttgtttagcattgaatctctgaaggctctatgaaaggaatagcatgatgtgctgttagaatcagatgttactgctaaaatttacatgttgtgatgtaaattgtgtagaaaaccattaaatcattcaaaataataaactatttttattagagaatgtatacttttagaaagctgtctccttatttaaataaaatagtgtttgtctgtagttcagtgttggggcaatcttgggggggattcttctctaatctttcagaaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttgaaggggagggaaagggggaaagcgggcaaccacttttccctagcttttccagaagcctgttaaaagcaaggtctccccacaagcaacttctctgccacatcgccaccccgtgccttttgatctagcacagacccttcacccctcacctcgatgcagccagtagcttggatccttgtgggcatgatccataatcggtttcaaggtaacgatggtgtcgaggtctttggtgggttgaactatgttagaaaaggccattaatttgcctgcaaattgttaacagaagggtattaaaaccacagctaagtagctctattataatacttatccagtgactaaaaccaacttaaaccagtaagtggagaaataacatgttcaagaactgtaatgctgggtgggaacatgtaacttgtagactggagaagataggcatttgagtggctgagagggcttttgggtgggaatgcaaaaattctctgctaagactttttcaggtgaacataacagacttggccaagctagcatcttagcggaagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgactactaatctgtcttcaggactctttctgtatttctccttttctctgcaggtgctagttcttggagttttggggaggtgggaggtaacagcacaatatctttgaactatatacatccttgatgtataatttgtcaggagcttgacttgattgtatattcatatttacacgagaacctaatataactgccttgtctttttcaggtaatagcctgcagctggtgttttgagaagccctactgctgaaaacttaacaattttgtgtaataaaaatggagaagctctaaattgttgtggttcttttgtgaataaaaaaatcttgattggggaaaaaa

SEQ ID NO: 135 - DNA, 703 bp, Homo sapiens
  source 1..703: mol_type = genomic DNA
gtagagatgggggtttcatcctgttggtcaggctggtcttgaactcctgacctcaagtgatctgcctaccttggcctcccaaaaggctgagattacaggcatgagccactgcgccaggccttctttcttttctttttttctttctttttttttttgagacatcatttagctgtgctgaggggttcttaaataggcagctcagaaaattgttttcctttgtcagccacataaattcagcagaggctcttggagggtccctgctggtgaggggtgaggccagcagtggaactctgatttggtttttgctgagctggtggttgaaaggaatcctactacatcggggttataatagggaagatacattttagaatatgcccagtggagccatcggatgctgcatcgtccccagagagccaagtcatcgtgggccaagctcccatccccatgtctggcctcaactgcaggcccagatgttgacagctgcctctggagggttatgggagcctgtgaatgccaacatccccattgcctgcagcggctgctcccatcctggcttcctggtgggacttttccatgaattggggaatctgctttctgattccaaggcctattaaaatttctgagcattgcccatttcttttgctttatctgtaggacatgggctgtttttaaagaacctcacaaatgaaaaaaaaaaaaaaaa

SEQ ID NO: 136 - DNA, 2273 bp, Homo sapiens
  source 1..2273: mol_type = genomic DNA
gcctgtgctctggagcttgatccgaaagcttccacagtgaggactgctccgtgggggtaagagagcaccaggcactgaggcctgggagttccacagaccaacacccctgctcctggcggctcccacccgggacttagaccctcaggtccctaatatcccggaggtgctctcaatcagaaaggtcctgctccgcttcgcagtggaatggaacggatttagaagcctgcagtaggggagtggggagtggagagagggagcccagagttacagacggcggcgagaggaaggaggggcgtctttatttttttaaggccccaaagagtctgatgtttacaagaccagaaatgccacggccgcgtcctggcagagaaaaggctgaaatggaggaccggcgccttccttataagtatgcacattggcgagagaagtgctgcaacctaaaccagcaattacacccaagctcgttggggcctaagccagtaccgacctggtagaaaaagcaaccacgaagctagagagagagccagaggagggaagagagcgccagacgaaggtgaaagcgaaccacgcagagaaatgcaggcaagggagcaaggcggcagttcccggaacaaacgtggcagagggcaagacgggcactcacagacagaggtttatgtatttttattttttaaaatctgatttggtgttccatgaggaaaagggaaaatctagggaacgggagtacagagagaataatccgggtcctagctcgccacatgaacgcccagagaacgctggaaaaacctgagcgggtgccggggcagcacccggctcgggtcagccactgccccacaccgggcccaccaagccccgcccctcgcggccaccggggcttccttgctcttcttatcatctccatctttatgatgaggcttgttaacaagaccagagagctggccaagcacctctatctcagccgcgcccgctcagccgagcagcggtcggtggggggactgggaggcgctaattaattgattcctttggactgtaaaatatggcggcgtctacacggaacccatggactcataaacaatatatctgttgggcgtgagtgcactgtctctcaaataatttttccataggcaaatgtcagagggttctggatttttagttgctaaggaaagatccaaatgggaccaattttaggaggcccaaacagagtccgttcagtgtcagaaaatgcttccccaaaggggttgggagtgtgttttgttggaaaaaagcttgggttataggaaagcctttccctgctacttgtgtagacccagcccaatttaagaattacaaggaagcgaaggggttgtgtaggccggaagcctctctgtcccggctggatgcaggggacttgagctgctccggaatttgagaggaacatagaagcaaaggtccagcctttgcttcgtgctgattcctagacttaagattcaaaaacaaatttttaaaagtgaaaccagccctagcctttggaagctcttgaaggttcagcacccacccaggaatccacctgcctgttacacgcctctccaagacacagtggcaccgcttttctaactggcagcacagagcaactctataatatgcttatattaggtctagaagaatgcatcttgagacacatgggtaacctaattatataatgcttgttccatacaggagtgattatgcagtgggaccctgctgcaaacgggactttgcactctaaatatagaccccagcttgggacaaaagttgcagtagaaaaatagacataggagaacacttaaataagtgatgcatgtagacacagaaggggtatttaaaagacagaaataatagaagtacagaagaacagaaaaaaaatcagcagatggagattaccattcccaatgcctgaacttcctcctgctattaagattgctagagaattgtgtcttaaacagttcatgaacccagaagaatgcaatttcaatgtatttagtacacacacagtatgtatataaacacaactcacagaatatattttccatacattgggtaggtatgcactttgtgtatatataataatgtattttccatgcagttttaaaatgtagatatattaatatctggatgcattttctgtgcactggttttatatgccttatggagtatatactcacatgtagctaaatagactcaggactgcacattccttgtgtaggttgtgtgtgtgtggtggttttatgcataaataaagttttacatgtggtgaa

SEQ ID NO: 137 - DNA, 3344 bp, Homo sapiens
  source 1..3344: mol_type = genomic DNA
ctcactctccgggcgggtgctggacggacggacggacggggcagccgtgctcacagctcagcagcgcggggccttggcgcgcggggcgcttccccgggtcgccgtcatggccgcggaggtggcacgcccgagcggcctcgcctgagctccgggggtcgtcgccccgcagggtaggtgtttgggtgctcgcggctgcggcgggcgggctgggggcagcggtggccgtgcattgccgcgctgcgaggacggcgctgggttcgcggccgcgaggagggtgtgcctttgccgcgccgcctacgtgcgggtccgggctccgcggggccgggtgcgggaccccgcagatcgtcacccgcaacccaggcagccccaccgcgagtgccgccggaccccctggacgccgctgccagaggcgttcgcgcctatctggtatgaggtccacagacccgattcttacaacctggcgctctaacctcgccaacgggccaggaaaaaaacagaacaaggaaaagaagaaaaagtctgttccaagtaataatgggactagacagtaactgtttgcactttcgtctcttatggaaaatatgattattttgatgctttagtattacagactgtatcagacatactcttaaaagtatttaaaaacgtggggtcctaaaaatttcctagatcgggacagctgataatgcgacttctgttagtctccgcatccaactttcagggcggaatggggatttctaccttttactgaatatactcctgtaattttgaatattttttctcatcaaatgtgttactttcatgttttaggaaaccaataaaaatcaaaaatagaaacacccagcttaggtcctccttcttccaggaatcctcctctaacccctccctcccaggcaggatcggaggcccctcccgggtgctttctggggcctcactgaggtcctggtcccctaagtcctcccccgcagcccctccgcgtattaacccaccggtctcgctgggctcagagattgcttacttagcacatggtaagtgattaatactgtttaatgaccgagggaaaagataaaatgttctctgatgtttcacgcttttcgctctgaagttaaatagtggtaaatttatatgcgcttagataagaacatttaacagttaaggatctggaaaagttgacgacgtccgatgtccgtgtttcctgatccagcagttgcacgcctgggtattcatccttaagaacaatgtttttcagaatttgaagtccattttagtgggtcgtaaagtcatttaaatggatgatcagaatttgtaaaacacagacggagaatagaacatcagtgtctctcaggtcataggggtgttcgctttatgaatctctctccccctcctcagtttcatacatacacaaatgtgtgtcatgatgtaaaacgtcattccttagtatgagtcatggacaaaaaagtttgaaagccactgccctagataaattttaatacatgtccacaagcagacgtgtacaagaaggttgactgtaatggcagaaaactggatgccgccttcatgcctacctgaaggagaaagataaattatacaccactcataaaaagggcacactgtacagtggccaatgaagtatttttgtgtttcaacacaaatgcctttcagtgttgaaccaaaaaaaaaaaaagttgccggagaatgctgtgtgatatcttctatgtaaaattcagaacctggtggagcaaagctgttgtttagggatgcatgtataggcattaaaagtagtaaagaaaaaaagtcaagctaatggtcatctcctagtggggaggagagggtcaggggctcagagatgctggaaatgctttatcgcctgacctgtgcaatggttacatgggtgtttgctttgtaactctttgttaaatgatacacactgggtttgaatgttacatttccccccaaatatgttaaaatatgggtgttgcaaactgaagaagcctgagtctagggctctccgtctgttattcttatttcctggcttctttttagaaaaaaaaaatcttgctcagggaggagtacctagggaatttatcagtaatgttttatttcataaattccatggtggacataccagtattcattaaattattctttatgtattcttataacttaatttataatatttcaaaaagcatttaggagaaaacatcatttggtgtgtttgctttcccaaaagtcacatttacatgttttgtacacaactggaatattttatatagtaattttagctaaaatcaaattttgttatgaaaaacgaaatactaaaaagtatataaaaatcacagaactttacagactttcagcgttttaaaatgctaacatattgtatttacctcaataatttctgttcctcagttctttcaggccacatttttagtgctcaatagcctcatatgactttaatattttattatattttattatgttatatattttacattatacattatagaacagaatatttccatcattgcagaaatttatattggaaaacattctggatacaaatatttgttatatacttgcagatattaaactgtagacattaatccttatgctttttgtcttaagatactctactattaatgaaatgtattctgaatatttttctaagaatttttaagtttatgttttgtccacattttgttctttaatttacctgagacatgtatatattgagatgtatatattttatgttatgagatcaagcttaattaattttcagattcccacatgaaaaggtaattgtcccaatatcagtgattagcagtgccacttctgtcacatacagtacccagatatgtttgggcttatttccaggctgttaatcctggtttatttgtcatttgaaaatactgcagattttcatttactgctgctttctatgtagtatattgttcagttctaagaatttaagattttttcattgtgatattttttcttcccctaagagatttgtagaaagtagtctagttccaatgtggaaggttttgtgagttatccttttgttactgatttctaatttgcactgtagttagcatacgtgtcaaggccaacaattctttgaaattcgagaattgctttatggttttaactgctccttgtatcatcaagaataatttgtactcacagacttttgaggcaatgtatgtaggtgtgtgtatatatataatatatacacatacactttatatatagattatatataatctctctgtagagtttatataaaaattaaatatatttatataaatgtatatatatatatgaataaaatctcctgtctctaa

SEQ ID NO: 138 - DNA, 812 bp, Homo sapiens
  source 1..812: mol_type = genomic DNA
atgtgtaggtggtcggtcccattgacaacctcaaatgagctgccagccaacaaccagcatcaactgccagtcagcttttgatgccattgtatctgacatcattctaacaggatcaaattgagcaacaacagcaacctaaaaatgagaaagcatggctacttggaacaagatgaagaaataaagcttacggaggttaagtaaatgacctgtttccagtgccggactccaggctctgctggttttacttctaatgcttccatcggaggacaacaatggttacattgacttaagatctgatgcaaatgtttaccttttggggtctgtcataccatgaagcaaacagacagaaaagaaggaaacagatggcacactgaaaattaggataagttaagaagaatgtaataagcggacaaccgacaaaggagggtgggaatgcagggcaaccgcaagggctcatacagtgctgggtgaggaggacccctgacgggagctgagatctttggtgaaggacacaactggtcagtacaaccctgcagggcaaggagctgcagaaacaactatccaaaccccacacctctccctcaccttgatctcccatgttccacttcggctgaaccaaaccaaaagccagagggcaaggaagccatgtgtgaaaactgtgctacagagcagaatggagagtgatacgactcagggagaagatatccagcactaaagtattaatgccttttattataaattgtttggaagtagatgatgtctatatgcataaataaataacactaatctcttttaaaaaaaaaaaaaaaaaa

SEQ ID NO: 139 - DNA, 491 bp, Homo sapiens
  source 1..491: mol_type = genomic DNA
tgcattttgcttgtttattatgttttctctccatcagaatgaaagctcctcgagggcagggactttggtctattgtctgtatttgccggtgcctaggattgtgcctgtatgcaacaggcactcaataaatatttttgctgtagactggacaggcatgagttagattctctggggcttctgcagagactggtttgggaaagtgggtgctagggaaaagctctgctccctgcaacctccccattttaatctttcagtattgaaaagtggagaggaaccggattcagtctgctggggacagaggcagtggggtgtggagaggcgggaccggggttggggagagtggggctcagcatgcgcatgcgcaattcgcgcgagcgcagtcaacatgtgattgatgagccagtctttttcctgggattcgcttttgcctttcttgcaaagtttcctggggaaagaagagcgagcaagtagaaaagggaaagtgggaggac

SEQ ID NO: 140 - DNA, 4338 bp, Homo sapiens
  source 1..4338: mol_type = genomic DNA
agagccggcggtgcgcgcgccttcgccgctgcctcccacccaccccctcgacgggagggtgaggcgcggcgcagtgatcgggcggccggggtcctgtgcgcgtgcgcagcgaacagctgtcacctagtgcggaacaagtctcccaaatttcccaaatctccctgggccggaggccactgtcttctcttcctcctccaccgagtcgtgctctcgccccaacccgcgcgccagacactgccctaaccatcatggaggtggccgaggtggaaagtcctctgaaccccagctgtaagataatgaccttcagaccctccatggaggagttccgggagttcaacaaataccttgcatacatggagtctaaaggagcccatcgtgcgggtcttgcaaaggtgattcctcctaaggagtggaagccaagacagtgctatgatgacattgataatttgctcattccagcaccaattcagcagatggtcacagggcagtcaggactgttcactcagtacaacatccagaaaaaagcgatgactgtgaaggagttcaggcagctggccaacagtggcaaatattgtactccaagatacttggattacgaagatttggagcgcaagtactggaagaacttaacttttgtggcacctatctatggtgcagatattaatgggagcatatatgatgagggtgtggatgaatggaacatagctcgcctcaatacagtcttggatgtggttgaagaagagtgtggcatttctattgagggtgtaaataccccatatctctattttggcatgtggaagaccacgtttgcatggcacaccgaagacatggacctctatagcattaattatctccactttggagagcccaagtcttggtatgctatacctccggagcatggaaaacgacttgaaagactagctcaaggttttttcccaagcagctcccaagggtgtgatgcatttcttcgccacaagatgacattgatttctccatcagtattgaagaaatatggtattccctttgacaagataacccaggaggctggagaattcatgatcactttcccatatggctaccatgctggttttaatcatggtttcaactgtgcagaatctacaaattttgctactgtcagatggattgactatggaaaagttgccaaattgtgcacttgcaggaaagacatggtgaagatttcaatggatatctttgtgaggaaatttcagccagacagatatcagctttggaaacaaggaaaggatatatacaccattgatcacacgaagcctactccagcatccacccctgaagtaaaagcatggctgcagaggaggaggaaagtaagaaaagcatcccgaagcttccagtgtgctaggtctacctctaaaaggcctaaggctgatgaggaagaggaagtgtcagatgaagtcgatggggcagaggtccctaaccccgactcagtcacagatgacctcaaggtcagtgaaaagtcagaagcagcagtgaagctgaggaacacagaagcatcttcagaagaagagtcatctgctagcaggatgcaggtggagcagaatttatcagatcatatcaaactctcaggaaacagctgcttaagtacatctgtaacagaagacataaaaactgaggatgacaaagcttatgcatatagaagtgtaccttctatatccagtgaggctgatgattccattccattgtctagtggctatgagaagcccgagaaatcagacccatccgagctttcatggccaaagtcacctgagtcatgctcatcagtggcagagagtaatggtgtgttaacagagggagaagagagtgatgtggagagccatgggaatggccttgaacctggggaaatcccagcggtccccagtggagagagaaatagcttcaaagtccccagtatagcagagggagagaacaaaacctctaagagttggcgccatccacttagcaggcctccagcaagatctccgatgactcttgtgaagcagcaggcgccaagtgatgaagaattgcctgaggttctgtccattgaggaggaagtggaagaaacagagtcttgggcgaaacctctcatccacctttggcagacgaagtcccctaacttcgcagctgagcaagagtataatgcaacagtggccaggatgaagccacactgtgccatctgcactctgctcatgccgtaccacaagccagatagcagcaatgaagaaaatgatgctagatgggagacaaaattagatgaagtcgttacatcggagggaaagactaagcccctcataccagagatgtgttttatttatagtgaagaaaatatagaatattctccacccaatgccttccttgaagaggatggaacaagtctccttatttcctgtgcaaagtgctgcgtacgggttcatgcaagttgttatggtattccttctcatgagatctgtgatggatggctgtgtgcccggtgcaaaagaaatgcgtggacagcagaatgctgtctctgcaatttgagaggaggtgctcttaagcaaacgaagaacaataagtgggcccatgtcatgtgcgccgttgcggtcccagaagttcgattcactaatgtcccagaaaggacacaaatagatgtaggcagaatacctttacagaggttaaaattgaaatgcatcttctgcagacaccgggttaagagggtctctggagcctgcatccagtgttcctacggtcgctgcccggcctccttccatgtcacttgtgcccatgctgctggggtactgatggagcctgatgactggccttatgtggtgaacattacatgctttcgacataaggtcaaccccaacgtgaagtccaaggcttgcgagaaggtcatttccgtgggtcaaacggtcatcacgaagcatcggaacacccggtattacagttgcagagtgatggctgtgacatcgcagaccttctatgaggtcatgtttgatgatggctcctttagcagagacacatttcctgaggatatcgtgagccgagactgtctgaagctgggcccacctgctgagggagaagtcgtccaagtcaagtggcccgatggcaaactctatggagcaaaatattttggatcaaatattgcccacatgtaccaggttgagtttgaagatggatcccagatagcaatgaagagagaggacatctacactttagatgaagagttacccaagagagtgaaagctcgattttccacagcctctgacatgcgatttgaagacacgttttatggagcagacattatccaaggggagagaaagagacaaagagtgctgagctccaggtttaagaatgaatatgtggccgaccctgtataccgcacttttttgaagagctctttccagaagaagtgccagaagagacagtagtctgcatacatcgctgcaggccacagagcagcttgggttggaagagagaagatgaagggacatccttggggctgtgccgtgagttttgctggcataggtgacagggtgtgtctctgacagtggtaaatcgggtttccagagtttggtcaccaaaaatacaaaatacacccaatgaattggacgcagcaatctgaaatcatctctagtcttgctttcacttgtgagcagttgtcttctatgatcccaaagaagttttctaagtgaaaggaaatactagtgaatcacccacaaggaaaagccactgccacagaggaggcgggtccccttgtgcggcttagggccctgtcaggaaacacacggggacctctctctctagctccagcaggtggcacctcggtacccagcgggtagggcgataatttatatattttccacagtcagggaaggactctcacttatttgtttcaaattgcagtttttataaaacatttttaaaacacaaatggcatgtatgctaatgagatttacccgtgtgctatctgtatttcccttgtacagaacttttacatttttgaatattcctattacttttgattgtgtctgatgggaactgagttgttggcctttgtgaaatgaaatttttggctcttgagaaagaattcttatgaattgttatgcgaattttatatatttaaagagggagatctggggctgttatttttaaacactttttttcataatacatattccgagtagatatttataaaatatatgtttctttcattatgtgtttgtaaaattagagtttaaataaatatgctttgatgcatagttttgaactaatgtaacatgatttttcttttttaaaacagcctgaaaatgtactagtgtttaaaaataaagatttccattttctcca

SEQ ID NO: 141 - DNA, 6866 bp, Homo sapiens
  source 1..6866: mol_type = genomic DNA
agtagctgtggtcggggctcgggaccggctgccatcttagtctagggactgaggagtcgccgccgccccgagtcccggtaccatgcatttcacggtggccttgtggagacaacgccttaacccaaggaagtgactcaaactgtgagaacttcaggttttccaacctattggtggtatgtctgacagtggatcacaacttggttcaatgggtagcctcaccatgaaatcacagcttcagatcactgtcatctcagcaaaacttaaggaaaataagaagaattggtttggaccaagtccttacgtagaggtcacagtagatggacagtcaaagaagacagaaaaatgcaacaacacaaacagtcccaagtggaagcaaccccttacagttatcgttacccctgtgagtaaattacattttcgtgtgtggagtcaccagacactgaaatctgatgttttgttgggaactgctgcattagatatttatgaaacattaaagtcaaacaatatgaaacttgaagaagtagttgtgactttgcagcttggaggtgacaaagagccaacagagacaataggagacttgtcaatttgtcttgatgggctacagttagagtctgaagttgttaccaatggtgaaactacatgttcagaaaatggggtttcgctctgtttgcccaggctggagtgcaatagtgcgatctcggctcactgcaacctctgcctcccgggtttaagtgattctcctatctcagcctcccgagtagctgggtttacaggtgcttctcagaatgatgatggctccagatccaaggatgaaacaagagtgagcacaaatggatcagatgaccctgaagatgcaggagctggtgaaaataggagagtcagtgggaataattctccatcactctcaaatggtggttttaaaccttctagacctccaagaccttcacgaccaccaccacccaccccacgtagaccagcatctgtcaatggttcaccatctgccacttctgaaagtgatgggtctagtacaggctctctgccgccgacaaatacaaatacaaatacatctgaaggagcaacatctggattaataattcctcttactatatctggaggctcaggccctaggccattaaatcctgtaactcaagctcccttgccacctggttgggagcagagagtggaccagcacgggcgagtttactatgtagatcatgttgagaaaagaacaacatgggatagaccagaacctctacctcctggctgggaacggcgggttgacaacatgggacgtatttattatgttgaccatttcacaagaacaacaacgtggcagaggccaacactggaatccgtccggaactatgaacaatggcagctacagcgtagtcagcttcaaggagcaatgcagcagtttaaccagagattcatttatgggaatcaagatttatttgctacatcacaaagtaaagaatttgatcctcttggtccattgccacctggatgggagaagagaacagacagcaatggcagagtatatttcgtcaaccacaacacacgaattacacaatgggaagaccccagaagtcaaggtcaattaaatgaaaagcccttacctgaaggttgggaaatgagattcacagtggatggaattccatattttgtggaccacaatagaagaactaccacctatatagatccccgcacaggaaaatctgccctagacaatggacctcagatagcctatgttcgggacttcaaagcaaaggttcagtatttccggttctggtgtcagcaactggccatgccacagcacataaagattacagtgacaagaaaaacattgtttgaggattcctttcaacagataatgagcttcagtccccaagatctgcgaagacgtttgtgggtgatttttccaggagaagaaggtttagattatggaggtgtagcaagagaatggttctttcttttgtcacatgaagtgttgaacccaatgtattgcctgtttgaatatgcagggaaggataactactgcttgcagataaaccccgcttcttacatcaatccagatcacctgaaatattttcgttttattggcagatttattgccatggctctgttccatgggaaattcatagacacgggtttttctttaccattctataagcgtatcttgaacaaaccagttggactcaaggatttagaatctattgatccagaattttacaattctctcatctgggttaaggaaaacaatattgaggaatgtgatttggaaatgtacttctccgttgacaaagaaattctaggtgaaattaagagtcatgatctgaaacctaatggtggcaatattcttgtaacagaagaaaataaagaggaatacatcagaatggtagctgagtggaggttgtctcgaggtgttgaagaacagacacaagctttctttgaaggctttaatgaaattcttccccagcaatatttgcaatactttgatgcaaaggaattagaggtccttttatgtggaatgcaagagattgatttgaatgactggcaaagacatgccatctaccgtcattatgcaaggaccagcaaacaaatcatgtggttttggcagtttgttaaagaaattgataatgagaagagaatgagacttctgcagtttgttactggaacctgccgattgccagtaggaggatttgctgatctcatggggagcaatggaccacagaaattctgcattgaaaaagttgggaaagaaaattggctacccagaagtcatacctgttttaatcgcctggacctgccaccatacaagagctatgagcaactgaaggaaaagctgttgtttgccatagaagaaacagaaggatttggacaagagtaacttctgagaacttgcaccatgaatgggcaagaacttatttgcaatgtttgtccttctctgcctgttgcacatcttgtaaaattggacaatggctctttagagagttatctgagtgtaagtaaattaatgttctcatttagatttatctcccagtgatttctactcagcgtttccagaaatcaggtctgcaaatgactagtcagaaccttgcttaacatgagattttaacacaacaatgaaatttgccttgtcttattccactagtttattcctttaacaacaatattttatgtgtgtcaaaagtctcacttgggagtagtgtttttttcttttagacattctgcagacatgcagggaagtcctttggtaactgcaatatacaagattttcctattaagcctcttggtaagaggcatttgttaaaagtgcaagcttactcctgcttctggggatgtgagcaaaattcgggcttgtgttctccctctcattttagtctgacttgactattgtttttcctttctggcgcatgaatccatacatcattcctggaagtgaggcaagactcttgcatctctacaaagtagttttgtcaatttgaattcagggaaaagttggtcacagcctgcaaatgacttcatttggaagtctgattgtttcagttgcctgacaaatactacactttacaaacaatgttaacactgtgattccttcattgttttaagaagttaacctagggccgggcatggtggctcatacctgtaatcctagcactctgggaggccgaggcaggaggatccctttagcccaggagttaaagaccagcctgggcaacatagggagaccctgtcttttttttgggcagcgtggtgggggataaataaataaaaggaaaaaaaacttagcctagaattagaattaatttaattgaattcatctaaagatgtctctggtgatttttatatgttccgctatataattgatgctttatagttttatcataatccaacaacttcagttatatttaattattgttaaggagtttaagactagaaagactagagtgctttctagtccaaatagaggtcagtgaaacagcttttgacatcagattttcatttgagagggagagctgtggtactggctaaaaagaaaggaagataacatccagtaaccacaggaatatattctctgtgaattaaaagtcttcaaagttatcatttctctgacatatgttggagtagtcatttccattctttacattgtcatgaactggattgataaccctcatctgcaatattttcacccctaaaatttttaacagggtttcctttttttctcacgactatttaagtttagattgctccattattaactgattaatgcactttgaagttctctggaattaattattttaacttggcctagcttcgactgtcaaggtggctgttataaatttgacttcattggcagtggatgaagcctaagccagctgagtctctatcatagctgaaccctgaggacagcctcatagctcatgtatcagggacttttgccacatttcagaggcatagcatgaacaagtaatattaagccaagaataagcagcagaaccctgttccatatggaaaaaagaaaaacaattttttgtccctaatgttcttccttttacatcctggaacaacaataaaaacatttttttaaacttgtctactgtaagatactgccatcataaagcagagacttacatgagtgaaagggttgcctcatcaagcagctcagtgtaaatggggaggctaggctctccccagccctatggtttttttatttcatgtaccccaggaaatactgtgtggtttctaaaagccctggttgttaaaagtagggactctgcctttttgttggtagggagaaaaaacgctattgctttgtcttacagagcgaatgtctgccaactacccgttcattatataagtctgaacttggtaatatatggctaatgaagattaagccctctataaagacttcctgttgaggtgaattctcatactgaaatgtagttacctacaatatttactagagatttatgaaattaaattaagagataatgtaagaaaatacatttttttggttctatataatgcttcatgattcatttagggacctagaaatattgtgtgaaaatatataaatatcacccaaaaggctttctgccctatatttttaaaatacagaatagttatatttgaagtagccctggccctagttctatagggcttggctatttaatatttttatggaagaagtgtttagttctggaaaaggtaaatgcttgtatatatttttgcagcctgggatctccctactccattttttcctttaatttaagtggccacatgtatatgtcttccctgctgtgttaggaaaatgggggctggatatcccaagaatcagaggttatataaaaatactgcaaatagaccgcagacataaatatctaccaaatgctatcttaaattttggtccaaactgaacatatggaaatagatttattgtaagtatttacttagagctttttcttaaatctgaactaacttgcttttagaagtctttttctttgtaagcattgtaaatgctaataaatcctgttaatttttttttttttttttttttttttttactgcatgttacattgaaataaaaacaaagtaaaatgagcaattgccttattgttctgctcttttgcagggggtgggggttggggaagacagcattccacagcctggtcaggagggaaggacctcacttgtttcatcaaatggagtggtttaaaactgatcaagcagctcataataatgaattcctgctctgtcttttattatcttatgtcagagtgcagcaaaatctcatttcataagacttacaataactctatttaaaattcaggtattgtactcgggaggctgaggtgggaggatcccaggagttcaaatcctgcctgggcaacatagcaagaccctgtctcttaaaaaaacaaaaagacatattcaggtactgatttttcatcaagagattgacataccaatgatcagaaattttagtgaaacttgactcaactatacacaaagttctcatcctaaatgaactatttcagtcaagcccactctaccactttttacttatctggttaataatccgaaatgttaccggggttgactcacggaattaaatttttcctctatttttatatcctttcctgttgtggatgtaacttacctttctaaactcctttagagttaacgacttctatttagtattgttggtatctccagcccccctcctttttttgtttttcttttagttatttactttgtttgtataatttgctttcattaactaaaaatggaattggcagcactggattgtatattactgtattaagagtacctttgttttcttgatctgttacttttaatactcaggtaatgctacaaatattacagggagtcaaatgcttttataaagttccttcagtggttgcctcacttgcgttttcctacaagatgtaagactgtttataattaaacttttttggtccttcatttgcatgcttttctatcagtcttgggtaaggaggggtctctttcctctctccccggccccaccttctgagacttatatgtgaccaggtcacatagatttctgcctctctgcttttttaagtggcaagttacagcccatctggattgtggtattgttcccagaccaaatcaagggttggactgcttattctcgcagcccaataatgaggtgcagatgaactgagaaaggagggagtttatttctgtaaccaggtatggggagaaagcctggaaatgatcccaagaccaactcaaaactacaaagttttccagagcttatttacattataagctatatgtctacatgtgagtgtacattcatctaaagacataagtgattc

SEQ ID NO: 142 - DNA, 5214 bp, Homo sapiens
  source 1..5214: mol_type = genomic DNA
gtctgggcttccgaatatgttttatgacggttgattttacaccaggaggtttgtctccgaggaagacccagggaactggatatctagcgagaacttcctccggattccccggcgcctcgggaaaatgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtctatgatggttgcgccatctgtgcacactaagaaatgggactctgaactggagctgctaagctaaagaaactgcttacagtttattttctgtgtaaaatgagtaggatgtctcttggaaatgttaagaaagaagaccctttgttgaaaaatgttgctctgggagacttactgcattgccgacagcacagatgtgaaggacatgagactaagagaaaccttgcaaactctataaagaaacttttgaaaaagtgtacatgaagaatgtagccctctccaaatcaaggatcttttggacctggctaatggagtgtttgaaaactgacatcagatttcttaatgtctgtcagaagacactaattccttaaatgaactactgctattttttttaaatcaaaaacttttcatttcagattttaaaaagggtaacttgtttttattgcatttgctgttgtttctataaatgactattgtaatgccaatatgacacagcttgtgaatgtttagtgtgctgctgttctgtgtacataaagtcatcaaagtggggtacagtaaagaggcttccaagcattactttaacctccctcaacaaggtatacctcagttccacggttgctaaattataaaattgaaaacactaacaaaatttgaataataaatcgatccatgttttgtaacaaattcactgtgttatttaaggaaaaaaaggtaagctatgcttagtgccaacaataagtggccattcgtaaagcagtgttttagcatttcttgtgctggcttgtaatgtagggaaaaaaagtgctgttttttgaaaagatggtgtcatttcccccttcttcccatgttttaaagccccatcttatatccagttcccaaaatttgcatacttacctaagtattttttttaggtgtgctgtgtttggggaatatttgaaaatttaaagcatgatttaaaattttttaaagtgagctgtgacactggaaagctcttcattttatcttttaaaatagagttttttctatttatatatgtaaaattgtagtgtatttcttttcaccaaacagtgtgtgggacattctttatcactgttttaggatcacctcaggaagtgtcgttacccagaattccccactgtctgctatgagacttgtaactttatcactatacttctgcttggtgccatcttgtcagagtaatatttgatgtctgtgatatgtaaagaattatcctaggataaagatattaaactttaagcagatttcagatgttactgctttaaaacaaatcagggataacaaattaaacgtataacttaaaatatgcaatgacatttagaggtaaccaatgttgatataggtagcatagcctagcctcctccccaaaattgcttttacaactaacactgatactaatttaggatagttcatgccttatccttgctaagaaaatggaattgatggtaggcaggtgctaaagtgcttttcaaaacaatattacgttagaatacaattggattcttcctcaaatttatacaggccaaaaagtaaaacattaattttctgaatttccagattaccaatcaattaatcaacaaatagccagtattatgctgtgtatttctgtcaggtcattttaaaatccatgttaattttataaaagaattttttacatgtcactgtcaggagctcactgtgaatgtgttgtcttcaaatggttatttaaccacacagtacactacattttacatatatgtacgtaatctctgggaatagtaaattaattatgttatttataaacaatacataggtcaacagactttaagcagggaggaaaagaagagtaatagcgtctgtgtgctgcagaccattcagaactgtcacgtgtgtccccatggtctcattcattgtattcctagcaattcccttttcaatgttgagttcacctctttatttcacaaagtacttggtctctcaatttcttgatctggttttgcttccatttaaaaactaatcaagaagggaaaatattgagaatgtgcatacaagaaaatcattaatttcctgaagatgaatttctacctgttgtgaacatttaactttctttttaaaagttaaacaaaaataaacaagggatattatgatgaatgtttggcttatgtgagtactagagataaaatttttaaacccagttattcacaatataaaatgttttcaagttagaaaaaatttttagaaatcctgggtattgtatttaactgtagctaaccaattttaaaacttgtattcttttgagaactattattaatagaaaaactttttataagcagtaaaataagaatgttccagtgactacctgtccttatacctagtcttgttaaaactttcttttgcagggtatttagtgtttggtttacagtcagtgcagagtgggcaagttaacagaaagtttgagctagagatactggaaaaaaaaaagatcaaagaatgagaaaaatggtgatccattttggggcaaactgagaccccccaaataactctttcctcatgtgtatggtgctcctcatgactcgtcttgtattttgcctttctgatacccatcagaactgctgctgctctaacttatactctttaccttgcccagatctccgcgtaaggaatgctttatgatcaacttgccataggactgatggattaaccagtgttcggctttatttgaagtctatgccctgcacagctcttgtatgtattttagatgctagaagtttttttagcatgtgatgtgtgattcttgtttgaattctaggtaccttgtgaattccagaaaaagagactgtgcttcacgattgttagtcccatgaacttgcactatctatctttcatggtgatgttttgaaaatacaatcaggaaaaaacccaacacctttggaatttaaaatagaatcatatcatgaaatttaaaaagaatctcttctgttgcatttcctcacccctaagtaacagctacatttaagtaaaatgcaggtggtaggggaaaaaaaaccatggcgagatggtggtttagtggaataaactgattactggtttttttgtttttttttttttttttaaagaaagaagcttcatcacagatactttccagtttctcttttatacttttttgaaagattactttttaggaacatttggtatgatatgcataaaattatttatccatttatgggcaaaatgatacaagtagcatcttgattgaacatcatttacctcagatattcaaccagcagtacgttttttatgcagtctcaacccatatcccatttgttacctctcagaatattggtaagcagttattttcgctttactctgtatttcttgtgttttgggcacaggttattgtactactgtcaaatcgtacttgctattttttctgcaagtatttaacagaaagcttaaaatccccataaaaccccaccttggataagtgattgttaaatattgtacaaataaaatgtatgctatccccattccatccccaagttaaataaaaaaatgaatacgg

SEQ ID NO: 143 - DNA, 7662 bp, Homo sapiens
  source 1..7662: mol_type = genomic DNA
aagccgccattgttccgccgagggaggacagcggggcctggcgctggcgccgagacgccgcttagcggccgccactggagacactccctcccgcctcccgggtctcctggcggcggcggagtgaggctgacagcggggaacctgggagacccctccgccctccccgcggtggcagcggccgatccccggctccggcgcgagggacggccgcgatgcgctcggcctgaggttacccggcccggcccttcctcgcttccctcgactattccactgcgtctccgcgccccggcgtcatcctgcgagtccctctgacgggagggaagatggctgcacggagctggcaggacgagctggcccagcaggccgaggagggctcggcccggctgcgggaaatgctctcggtcggcctaggctttctgcgcaccgagctgggcctcgacctggggctggagccgaaacggtaccccggctgggtgatcctggtgggcactggcgcgctcgggctgctgctgctgtttctgctgggctacggctgggccgcggcttgcgccggcgcccgcaaaaagcggaggagcccgccccgcaagcgggaggaggcggcggccgtgccggccgcggcccccgacgacctggccttgctgaagaatctccggagcgaggaacagaagaagaagaaccggaagaaactgtccgagaagcccaaaccaaatgggcggactgttgaagtggctgagggtgaagctgttcgaacacctcaaagtgtaacagcaaagcagccaccagagattgacaagaaaaatgaaaagtcaaagaaaaataagaagaaatcaaagtcagatgctaaagcagtgcaaaacagttcacgccatgatggaaaggaagttgatgaaggagcctgggaaactaaaattagtcacagagagaaacgacagcagcgtaaacgtgataaggtgctgactgattctggttcattggattcaactatccctgggatagaaaataccatcacagttaccaccgagcaacttacaaccgcatcatttcctgttggttccaagaagaataaaggtgattctcatctaaatgttcaagttagcaactttaaatctggaaaaggagattctacacttcaggtttcttcaggattgaatgaaaacctcactgtcaatggaggaggctggaatgaaaagtctgtaaaactctcctcacagatcagtgcaggtgaggagaagtggaactccgtttcacctgcttctgcaggaaagaggaaaactgagccatctgcctggagtcaagacactggagatgctaatacaaatggaaaagactggggaaggagttggagtgaccgttcaatattttctggcattgggtctactgctgagccagtttctcagtctaccacttctgattatcagtgggatgttagccgtaatcaaccctatatcgatgatgaatggtctgggttaaatggtctgtcttctgctgatcccaactctgattggaatgcaccagcagaagagtggggcaattgggtagacgaagaaagagcttcacttctaaagtcccaggaaccaattcctgatgatcaaaaggtctcagatgatgataaagaaaagggagagggagctcttccaactgggaaatccaaaaagaaaaaaaagaaaaagaagaagcaaggtgaagataactctactgcacaggacacagaagaattagaaaaagagattagagaagaccttccagtgaatacctctaaaacccgtccaaaacaggaaaaagctttttccttgaagaccataagcactagtgatccagccgaagtactcgtcaaaaatagccagcctatcaagactcttccacctgctacttctaccgagccatctgtaatcttatcaaaaagtgattctgacaagagctcttcccaagtgccgccaatactacaagagacagataaatccaagtcaaataccaagcaaaatagtgtgcctccttcacagaccaagtctgaaactagctgggaatctcccaaacaaataaaaaagaagaaaaaagccagacgagaaacgtgaaattttttttcctgaattggacatgtgtttgcaaacacttgtcttgaagattatgctgtttatgcaataatttgtgaacatgtacagagttttatataaatttaaaccaatttttaaaacaaaactgcggacaccaccataaaaatggaatcaaaagaaagttaatttatgaaattaagaggtcagcagaatatactcagtgatggaagacacttgggaaagtctttttaatagaacaagaacgatcttaatttaagaatattatcctggtttaacaacagtgccctgtttacaacagattgtgccctatctcatctgcagccgaggaataaaggattctgattagaaagagggttgcctacagattagtaagcaattccttggatcttatgcacagaacttgtaccatttgaatctgttttatgcttaaatcaaagtgctttgatcaaatgcataacctgccatatctttacatatttgttggtagcaatttgtattaaagaaatcacaagtgcaaataaaaagtcatttatcatttgtttaactaaactgtcatggtttagtttacaatttttaaaaagttcttaaaatactgaaaatgcagttgacacttgtgtatggcttatgaagttatttttgatagtcttacattacttgaattgttcaaagtacagtatattttaaattaagaaaagtgaactatatgtatttgttttatacatttaaggcttagactcataaataatgctattgtttatgatttgaaaactttcaggcaaaatccaatttacatttttcccttccctagcaattacttttttccagcttcaactcttcttagttactaatactttgttgactttaaaaatgaaatcattcacaaacttttggtatatgatggagaatgaaaaactagagtcagacagctttaattgacattgtcaacacctccagttatcaggaatacatttttttactgccttaacctgtagtgcgtagaatatgcatcaatttcttgaaggagattcatgtttttataagaattttcatgtaattattgcaattgtggtcaaataaggaacgtttcctgcttgaaattatattgatttaaatgatgtgtgagatgtttcaccattttcaggcactgtgtaattctattgtaataaactggcaggtatctttgtaactataaatagtgcatgctcagccatgtacactgtaaatagcctttaccaaacgtgtttgacaaggaccataattaacatcacttagtgaattgtgataaagaaaaaaaagccatgatttattcgatgtgattggcttgtttttatgtggcgccaagaacgaacctgtttaacagctgtaaccaatggtactgatctatccatccaatgttgtcattatatttgactgtggttcaacagtattgcgttgtcagactaggaaagctaaacgaacaaaatggttttagttttgctgaagactggccttattaatggacagctttcctaacaagagattattaacttttatcaggtgttaacatctgtttcaggaacatggcagtatgtttacatgtcagaagttttgtttaattctatggtatttctaaattgacttgtttaaataaattcagcaaatggatagcattgttttttatttgcttcaatatgggggtagataatagctaaagagccaaggatgaatttcttcaaatgactttattctgttagctttacataggtgttggaggattcctaaggtgtcagcattttgtaaaggtaccacaaaggagaagttgatagggaatctaattttagaatgtgccaaatggtctgtgctcaacaatataattgaactctctcaactctacctcaccatttctttatctcaaaattctgttggctttgtcaaacgttggattttatttctgcagcctagtatctccccattctaccacctatgccagatgttaaagacagcagatgttttgaggagaatggcattgtggagatgcagagatgcgttgctaagttgaggtggatccagtatagagatacctctatttcttctttatggctcaagagctaggacttggattttgttttaagagatggcagctggccgggtgcagtggctcacgtctgtaatcccagcactttgggaggccgaggcaggtggatcacgaggtcaggagttcaagaccagcctggccaagagggtgaaaccccatctttactaaaaatacaaaaaaaaaaattagctgggtgtggtggtgggctcctgtaatcccagctactcaggaggctaaggcagagaatcatttgaacccaggagacggagtttgcaaagccaagatcgtgccactgcactccagcctgggcaacagaaggagactccgtctcaaaaaaaaaaaaaagatggcagctatataaatgataaaattaattacattctctttcacatgcatgaggtgcaaactctgtcacaaagtattttaattaccttttaccttgtttcatagatctttatgtgacataaaaacagtttctggcacggtggctcacgcctgtaatcctagcactttgggaggctgaggcaggtggatcacctgaggtcaggagtttgagaccagcctggccaatatggtgaaaccccatctctacaaaatttgcaaaaagtagatgggtgtggtagtgggcgcctgtaatcccagctactcaggaggttgaggcagagaatcgcttgaacccggggggtggaggttgcagtgagctgagatcgcaccactgcactccagcatgaaagagcgagactcaatctcaaaaaaaaaaaagtttctggcacctgaacaggaactggtttccatcatcaactcagaaagcactaaaatctaggtggtgattcagggaggagcaggggaagacagcctcctatggtggcatgaataagatgcttccagaactagtagggaaataactaacctcttcaggctttatcaggcctggaggggaaccttgctcatgttagcaagaaaggtatcctagagaagccactcaaaaggctccctaatccagcctgtctccacatacatactgaaaattcttccctactctgaggcagggtgtagtggtttaggggtttctccagactggaatcctacctatctgtaccgacaattgagcaaacaacagttgagagagtccaaaaaaaaaagtattaaaatgtgattgatgtaatttaccatgtttactttatgcatgcattttattggggaggggaggtcagaataattcacccaaatctagtggtcttatttcataggctaatctggtttatatttgcattaaagatactggagggcaatatttacagagtttagtttttcttaattaaaaacagtcctctattaatatagtgtgaaatatctttcaaaatttagagtttaggtttaagatgtctactagatatctttaagattttcctgtaaactcactgcacaaactggaattactttccaaaagacttagggaatgcaaatatgttactcataagatgcattgagtattgtaaataaaacaaaccatttttgatttgtttaaattgctcgttacagttctcttgtggggagggactttgtcagtcattttgcatcttaagctagactaaactttttgttgttgttttcctaaaaccataggtgcaagctttgccgctgggaagtcatattgaattaagcacttttgaaaatgtcactgtttgtgacacacaatgttctctacagaaaacttcttctatcttatgtcattttagcagtagatgtagctgcctgccaccaggtgcaatatgtcatttgcatcagccttttcagttaaggaaattaaaacttggcatgcattagttcatatgtatgaggttggctgtgtccccaaacagcaattgcttgtacaagatagaagtttgcttctcagctgggcatggtggctcatgcctgtaatccaagctctttgggaggccaacgcgggaggattgtttgagcccaggagtttgaaaccatcctgagcaatagagagacccccatctcgacaaaaaaaaaaaaaaaaaattagaaacaaaaaaaagattgtttctctttcatattaaagtttgggaaggcaatccagagcccatatgacattatgcagggtcagggatccgggttctttctatattgttcctccccattctcaacctgtagctttttttttttttcttttaatgagatagggtctcactctgttgcccaggctggagtgcagtggcacacttctggctcacttcagcctggaactcccgggctcgagctatattctcccactttagcctcagcctccagagtacctgggactacaggtacacaccaccatgcctggctaaattttttttttttttttttggtagagatgaggtcattgctatgttgcccaggctggccttgaagtccagggcttaagtgatcctcctgccttggcctcccaaagtgctggtgttacaggtgtgagccaccacacctggccaacatgtggctttcagttcttacttgaagacagctgctctagctagaaacttcctatcggcatctgagccagctggtagagggcattacctcccccacccccaagaacacttcttggaagttacacacaatacataaacttcaatttcaaacagttgcttggtcccactagccatgaagctggatgggacgtaatattctatctgggttgtcatgggccaaattaacattttgagattctgttaggaagatgagaattggataatgggcacctctaacagtgtctgtcagagttgacatacattgtgtatctcctgcaccaaagcatttttgagatcaagtgttcttccagctgagcagaaatttggaaggctattcagtgctgcttagtgtagcagctaataatgttccaacttctatatgatagtttgctttttgttttttgtttttttctagagacagagtctcgttgcccaggctggtctccaattcctgggttcaagcaatcctcctgactcagcctcctaaatgctgggattacaggcatgaaccactgtgcccagtctatatataatagattttagaagaaatttctgccactcagtgactgctttaaattctagagacagaggctgagagaaacttagtagcctgcctgcgcatactgcaagtacagactatataacaagttgaaagagaatcaccctggtatataatattttaaaacatgaaaaaggcattactattgttctggtctaagtactctaacaggacgatgatttctaaccctagcatcatgacatgtatatagtgtgttgtaagacatttgccagctaatagttacagtactgaagcttgtgaatacgttaattgagaaaactttaagcaggttcaaagttatcccatgatagtggcattcttatttgtatatatgtgctttccttttttatttgtatcaaggcatttaaaatgaatatatgtaacatgtacctaagttttaaaagcataataaaatgaacacatctgaaaccacca

SEQ ID NO: 144 - DNA, 1972 bp, Homo sapiens
  source 1..1972: mol_type = genomic DNA
gccacatcccggcagccctcctacctgcgcacgtggtgccgccgctgctgcctcccgctcgccctgaacccagtgcctgcagccatggctcccggccagctcgccttatttagtgtctctgacaaaaccggccttgtggaatttgcaagaaacctgaccgctcttggtttgaatctggtcgcttccggagggactgcaaaagctctcagggatgctggtctggcagtcagagatgtctctgagttgacgggatttcctgaaatgttggggggacgtgtgaaaactttgcatcctgcagtccatgctggaatcctagctcgtaatattccagaagataatgctgacatggccagacttgatttcaatcttataagagttgttgcctgcaatctctatccctttgtaaagacagtggcttctccaggtgtaactgttgaggaggctgtggagcaaattgacattggtggagtaaccttactgagagctgcagccaaaaaccacgctcgagtgacagtggtgtgtgaaccagaggactatgtggtggtgtccacggagatgcagagctccgagagtaaggacacctccttggagactagacgccagttagccttgaaggcattcactcatacggcacaatatgatgaagcaatttcagattatttcaggaaacagtacagcaaaggcgtatctcagatgcccttgagatatggaatgaacccacatcagacccctgcccagctgtacacactgcagcccaagcttcccatcacagttctaaatggagcccctggatttataaacttgtgcgatgctttgaacgcctggcagctggtgaaggaactcaaggaggctttaggtattccagccgctgcctctttcaaacatgtcagcccagcaggtgctgctgttggaattccactcagtgaagatgaggccaaagtctgcatggtttatgatctctataaaaccctcacacccatctcagcggcatatgcaagagcaagaggggctgataggatgtcttcatttggtgattttgttgcattgtccgatgtttgtgatgtaccaactgcaaaaattatttccagagaagtatctgatggtataattgccccaggatatgaagaagaagccttgacaatactttccaaaaagaaaaatggaaactattgtgtccttcagatggaccaatcttacaaaccagatgaaaatgaagttcgaactctctttggtcttcatttaagccagaagagaaataatggtgtcgtcgacaagtcattatttagcaatgttgttaccaaaaataaagatttgccagagtctgccctccgagacctcatcgtagccaccattgctgtcaagtacactcagtctaactctgtgtgctacgccaagaacgggcaggttatcggcattggagcaggacagcagtctcgtatacactgcactcgccttgcaggagataaggcaaactattggtggcttagacaccatccacaagtgctttcgatgaagtttaaaacaggagtgaagagagcagaaatctccaatgccatcgatcaatatgtgactggaaccattggcgaggatgaagatttgataaagtggaaggcactgtttgaggaagtccctgagttactcactgaggcagagaagaaggaatgggttgagaaactgactgaagtttctatcagctctgatgccttcttccctttccgagataacgtagacagagctaaaaggagtggtgtggcgtacattgcggctccctccggttctgctgctgacaaagttgtgattgaggcctgcgacgaactgggaatcatcctcgctcatacgaaccttcggctcttccaccactgattttaccacacactgttttttggcttgcttatgtgtaggtgaacagtcacgcctgaaactttgaggataactttttaaaaaaataaaacagtatctcttaatcactgga

SEQ ID NO: 145 - DNA, 4751 bp, Homo sapiens
  source 1..4751: mol_type = genomic DNA
gtggctatataaacatggctggcgtggccgcgcggcggggccgtgccaatcgcgcgtagggggctgtgggcactcggggttcgtagttttgaaatttctggcgggggagcagctgcgcagttaggctcgaggtgtgagcgacgaccttccgggagccgcaagtccaggctcccccgcagcgggacccgagcctgaggcgcagggctgaggcagcgcacgtgtgagcgccgctgaggaagctgcgagaggtcgggcgggtgtcgctccgggggcagcccaggctcgcgcggacgagaggaaggtccgggacgcgcgtgtcctgccgtgcagcgggcgcccgtcactgacttcgctgctgcggcccccgcgcctctccccagcgatgctgtggaacccgaaccgcaccggagtcggctgccgcgcgccaagcctcccctcacctctgctcccggaaccgcagcgccaaagctgccgctgagcccctgggggatgcccgccggcctcaccgaacccgccggcgccgctcccccggctgctgtgagcgcctcggggaccgtgaccatggccccggccggggcgctgccggtgcgggtggagagcactccggtggccctgggcgccgtgactaaggctcctgtcagcgtctgcgtggagcccacggcgtcccagcccctgcggtcccccgtggggaccctggtgaccaaagtggctccggtcagcgcccctcctaaagtcagcagcggccctaggctgcctgctcctcagatagtcgccgtgaaagcccccaacaccacgacaatccagtttcctgctaatttgcagcttcctccaggaaccgttttgattaaaagtaacagtggtccgttgatgttggtatctcctcagcaaactgtaacaagagccgagaccacaagtaacataacctcaaggccagcagtaccagcgaatcctcaaacagtcaaaatctgtacagtgccgaactctagctcacaattaatcaagaaagtggcagtgacacctgttaaaaaattggcacaaataggaactactgtggtaaccactgttccgaagccttcctcagtacaatctgtggctgtgccaaccagtgtcgtcacagttactcctggaaagccattgaatactgtaactaccctgaagccttcaagtttgggagcatcatccactccttcaaatgagcccaatcttaaagcagagaactcagcagctgttcagattaatctttctccgacaatgctagaaaatgtgaagaaatgcaagaacttccttgcaatgttaataaaactagcatgtagtggatcacagtccccagaaatggggcaaaatgtgaagaagctggtggaacaacttttggatgcaaaaatcgaagcagaagaatttactaggaaactgtatgttgaactcaagtcttcacctcagcctcacctggttccttttcttaagaaaagcgtggttgccttacgacaacttctgcctaactcccagagcttcatccagcaatgtgttcagcagacttctagtgacatggtcattgctacctgtactacaacagtaacaacttctcctgtggtgacaactacagtgtcctcaagccagtctgaaaagtcaattattgtttctggagcaacagcacccagaactgtgtcagtgcaaactttgaacccacttgctggtccagtgggagcaaaagctggagttgtgacacttcattctgtgggcccaactgctgcaacaggaggaacaacagctggaactggtttgcttcagacttcaaaaccacttgtgacatctgtggcaaacacagtgaccacggtctcactgcaacctgaaaagccagttgtctctggaacagcagtaacactgtcccttccagcagtaacttttggagaaacttcaggtgcagctatttgtcttccatctgtgaaacctgttgtttcttctgctgggaccacatctgacaagcctgttattgggactccagttcaaatcaaacttgcccagccgggccctgtcctttcacaaccagctgggattccacaggcagttcaagtcaagcaactagtagttcagcagccttcaggaggcaatgaaaaacaagtgaccacaatttcacattcctcaacattgaccattcagaaatgtggacagaagacgatgccagtgaacaccataatacctactagtcagtttcctccagcttccattctaaagcaaattactctgcctggaaataaaattctgtcacttcaagcatctcctactcagaaaaatagaataaaagagaatgtaacatcatgcttccgagatgaggatgacatcaatgatgtgacttctatggcaggggtcaaccttaatgaagaaaatgcctgcatcttagcaacaaactctgaattggttggcacactcattcagtcatgtaaagatgaaccatttctttttattggagctctacaaaagagaattttagacattggtaaaaagcatgacattacagaacttaactctgatgctgtgaacttgatctcccaagcaacacaggaacgactacgaggccttctagaaaaactgactgcaattgctcagcatcgaatgactacttacaaggcaagtgaaaattacatcctgtgtagtgataccaggtcacagctcaaatttcttgaaaagctggatcaattggagaagcagagaaaggatttggaagaaagagaaatgttacttaaggcagccaagagtcgttctaataaagaagatccagaacagctgagattaaagcagaaagccaaagagttacagcaattggaacttgcacagatacagcatagagacgctaatctcacagctcttgcagctattggaccaaggaagaagagaccactagaatctggaattgagggcttaaaagacaaccttcttgcttctgggacatccagcctgacagccaccaaacagttgcatcgtccaagaatcacgagaatctgcctcagggacttgatattttgtatggaacaggaacgggagatgaagtattctcgagctctatacctggcccttctgaagtgaccactccactcttccatccagatccttgctatttactgccaaagaagacacaaagcattgttgcactgtcctgaaatttcaatttctggaaaataatcaccaacatgaaagagcattgtttacagttagaaactttattaactcttacctatccatctcatgggactcttacagactcagattcatctttgtcttctgaaaatcagttatgaaatacactttgcacagaattaggcatctgcctatctgtgcattaaattaaagcaagttaaggccctatttgttactacctgcctcatttgccaaattgtagcaggtgaggtgtccttccctaatacagcatgcattgagatgcaggagaaaggaagaggcataagaaaatacaagaatgactatttcattctatattgaacctgtcccaaagtggtaggttttctgggctggaatcagaagttgcttagctaactatgacagtcaccctctgtacgtgacaggccactgtggtgactttggtcggctgagttccagtgttgtcaaaacaagacaaatggggatagattgtgcctggggttgctaattctctgcttcatatcaccatatgccgtttctggtaccaagtgaggggacatgcttgtcagggaaaccttaaaattgaatctactgatatatcttgttgaaaaaatggaaaaggtgcaaagtattagaaggaacgttagggcgttgctttcaaactgatgaggggaatgtgaaaatggtatcataaaacaggaaatgtgcttcatatgcccaagaggggaagaggtggaatcggaactccaaaacaactggtacatgttttgttgatgggcaaaaaaaatcacagcacaaccagaaaggagaatgtaccagatgttttcaaattagagtatttcaaaaggaaaatgttattttttgttttgtattgttttaaatcccaaggatagtttttatagttccttctgtcataatatattttagttatcctaatttttaagcatgttggcacatttaaaaaatcctaattgatgatgagaaatttttaacaataggactttatgttggattctgtacttaaaaggtactgtcaatatctcactcttggggatatgagctctaaatatgagaagcaagttatatgtgttcacctgaggaagaagctgacggacaaagcaaaccctctttaaaaaaaaaaaaaagaaaattccacgtgtgtggaatacagccggtgaaaaataatcactgtagttagaatcacatacagaaaaaaaatgattgaatcctccaagaaggtatttcaaaaatgggaaatgctatacttataagaattgaaggaaaaaaaaaaaaacacctcacgtatgttattcttcatcctgttcttcccattgggactgttatggccatattctacagtatagtaaattttaaattggccacatttttttttgtttcagcaagttatgtaaaatgctataaattatgtgtatgttaaaggagtactttaagaagagatctttatccaaagttgtttttgtatatttaaatgcttacctttattttttgtaaagtgcagcatactcatttgtgtataatcttactgatcagatgtttaaaattattccaaatacttcattaaaataattattttattataagaa

SEQ ID NO: 146 - DNA, 4298 bp, Homo sapiens
  source 1..4298: mol_type = genomic DNA
atcaatcgtcttccggcgcagccccgtccctgttttttgtgctcctccgagctcgctgttcgtccgggttttttacgttttaatttccaggacttgaactgccatgtcctctgaagaaggaaagctcttcgtgggagggctcaactttaacaccgacgagcaggcactggaagaccacttcagcagtttcggacctatctctgaggtggtcgttgtcaaggaccgggagactcagcggtccaggggttttggtttcatcaccttcaccaacccagagcatgcttcagttgccatgagagccatgaacggagagtctctggatggtcgtcagatccgtgtggatcatgcaggcaagtctgctcggggaaccagaggaggtggctttggggcccatgggcgtggtcgcagctactctagaggtggtggggaccagggctatgggagtggcaggtattatgacagtcgacctggagggtatggatatggatatggacgttccagagactataatggcagaaaccagggtggttatgaccgctactcaggaggaaattacagagacaattatgacaactgaaatgagacatgcacataatatagatacacaaggaataatttctgatccaggatcgtccttccaaatggctgtatttataaaggtttttggagctgcaccgaagcatcttattttatagtatatcaaccttttgtttttaaattgacctgccaaggtagctgaagaccttttagacagttccatctttttttttaaattttttctgcctatttaaagacaaattatgggacgtttgtagaacctgagtatttttctttttaccagttttttagtttgagctcttaggtttattggagctagcaataattggttctggcaagtttggccagactgacttcaaaaaattaatgtgtatccagggacattttaaaaacctgtacacagtgtttattgtggttaggaagcaatttcccaatgtacctataagaaatgtgcatcaagccagcctgaccaacatggtgaaaccccatctgtactaaacataaaaaaattagcctggcatggtggtgtacgcctgtaatcccagtgacttgggaggctgaggcaggagaatcgcttgaacccgggaggcggaggttgcagtgagctaagatcgcgccactgtactccagcctgggcaacagcgagactccatctcaaaaaaaaaggaaatgtgtatcaagaacatgattatccagcggtattttctaattcagatcatcaaactgattatatagaagagttggctttaaaatgtttgcaaatgtctttttttttttaatactggaagaaaaaatattctgttgtgtctcatacagtgcttaggatgtctttcacagagcttattaaaaagatgaaacctgagaacaaactgctttattcttactcagcccattttgcaaattaaaagtgggggcagaggtgggcggatcacctgaggtcaggagttcgagaccagcctggccaacagggcaaaaccccatctctactaaaaatacaaaagttagcggggcgtggtggcgggtgcatgtaatcccagctactcgggaggctgaggcaggagaattgcttgaacccaggaggcggaagttgcagtgagctgagattgtgccactgcactccagcctaggtgacagcaagactctgtctcaaaaaaaaaaaaatggcacatcaacgagagggaggcttggagaatatttggttggtgggtgggcagggactgccagagggttaggtgtacattgaggcctgaggcctgctggaattgggtttccttaactggtctcttatcttagtcccattgttctgagaacataagcacctaatctcatggcgtgagctaatccctatcataaatagtggtaccagttcattccttcccctgaaatgatgggggttgggcagagatgcactgaactcatctttgtgtatgagggccatgagagaggcctggtttgaaaacaatgctaagagctcagattccaggatttatgtaggggctcttctacttcccaggtatttgacctcaggtgagtcatttaatttcagccttatgccattttacattttctgtggagggttaggttaattggcatataaagccactgagaagagttggcaaagctctcagtcacggggatttgatcttttttggtgtgtgtgtgtcttgggtggtaaagtggtaattttgtaattctagctggtcttagttcctcagttctgctccctttagtcatggttctttctagtggctgtattgaggccccatgggtgttatccctccattggttctagtttggaacagaaaaatctgttgtattcatggctttcacctggctaatactgagctaagttaccaccaggttgcaaactccaggacattattgtcctgagctgcctattcccttgctggtgcgttgtggaaccctgtattattagttccagtcctggaggcctgcctcctgagtttcccagctagttgggacaggccccaatatcccttcttcctgtaacccaaacagtcatgaatttgctttggcaggtggatggagacttgggaacttccacctcacccactaagccaggcccaggctatggggcattgtggctaaccccaccaggtggatacttggtctgaggacgcatcttattctgggcctttagggagctaaggcagtgagaattggcaggaggactgctgtgaatgccttgtaggtcgggggattggagggggtcctctgctccctgtctctcaggatggcaatgtacctctcacccccagtgtcagctgaggtaggatccataatcagtaatattcctgcaacaaaatgttcctaagtggaatcaatgaagaccaaaatttatttgtataacaactctaaacctgctctgctctgctgtgtttacaatgtgctttgtgatcatccagcccagggagtcccagtctctggctgtctatctgctctaaggaagagagcccacagttctctatagtgccatagtctgtgatgaataaagttccagatttgaggtcaacccccgaccacccttaaagtgcttgttggtctcctactttggtttgtcttcagcatccaactgatgcacatttgtcaagaacccactgaggctagaaaccccccaaacactcaaggtaccttgagaacatgtatcccaagacctgcaaagaccaaacacatggcctgtaactacttttcactttcaagccctggaacttgtctgtaccaaaaatctttctgaaccattcaccaaatctcttgtcaagggccagaataaattaagagattggcttttggttttcaattaggctttcaagtcctaagcccaacccctcctatcccacctaggcagaaatcacttcagatccttgcttcctagtttaatttcttctctgcctcagttataacatccataaaatggaaataataatacccacctcacacaggggttaggagtgtttttttgttggtttttttccagacagggtctcactctgtctcccaggctggagtgcagtggcacaatctcggctcactgtaccctccgcctcccaggttcaagcagttctcatgcctcagcctcccaagtagctgggattacaggtgcctgccaccacgcccggctaatttttgtattaatagagacagggtttcaccgtgttgggcaggctggtcctgaactcctgacctcaagtgatccacccgcctcggcctcccaaagtgctgggattacaggcgtgagccaccacacctggcctaggagttaagagtattaaatgttaagaacagaccaagtccatcaaacagctccacacccttgttccatgttctggattggggagttgtgggggtggaggtgtagaacttttaaaaagcttcttaaaataagttgctgtgaatacttcaggtatataaaaaaacattggttgacaataagccagtgttctgccattcttacctgcttatcaagacaaaacctgctcaagtccctgcccagctgcattccaagtgttttcaagtttggtggtaagacctgacctgaggcttcttataacctttactcagtgggaatatgcatacagttcactacagaaatatttgtatttacttagaggaagtgccctggatctgggggcgggggggggcggggggaatgggtcttttctaaattgttaaaagcagttcatgccattattcttaataaacatttctaatatgctgtgaaa

SEQ ID NO: 147 - DNA, 8405 bp, Homo sapiens
  source 1..8405: mol_type = genomic DNA
acactcgcactcgcgcacacccgccgctcccactcacccgcgccgctctcccgccttccccgcgcgccccgcggccgcccgcgccgcaccatgcccgcggcgggcgcaccccgcgcccagcccggccgctcgcggtagccgtagccgcccgcggccgcgcggagcggggagccgccagcctgcgccccgtccgcgggtctatgggaagttcggggacttgacagccgctgccgccgcagggcatttttggtcgaagagagctgaagtaatgagaagacatcatggaggcccagtcccacagctccacgaccactgaaaagaaaaaagttgagaattccatagtgaagtgctccactcgaacagatgtgagcgagaaagccgttgcctccagcaccacttctaatgaggatgaaagtcctggacagacttatcacagagagagaagaaacgcaatcactatgcagccacagaatgtccaggggctcagcaaagtcagtgaggaaccttcaacatcgagtgacgagagggcctcattgatcaagaaagagatccatgggtccctgccacacgtggcggagccctctgtgccgtaccgcgggacggtgtttgccatggaccccaggaatggttacatggagccccactaccaccctcctcatcttttccctgccttccatcctcctgtaccaattgatgccagacatcatgagggccgttaccattacgatccatctccgattcctccattgcatatgacttccgccttatctagtagccctacgtatccggacctgcccttcattaggatctccccacaccggaaccccactgctgcttccgagtctcccttcagccctccacatccctacattaatccctacatggactatatccgctccttgcacagcagcccatcgctctccatgatctcagcaacccgtgggctgagccctacagatgcgccccatgcaggagtcagcccagcagaatactatcatcagatggccctgctaactggccagcgcagcccctatgcagacattattccctcagctgccaccgccggcacgggggccatccacatggaatatcttcatgctatggatagcaccagattctccagccccaggctgtcagccaggccgagccgaaaacgtacactgtccatatcaccactctccgatcatagctttgaccttcagaccatgataaggacgtctcccaactccttggtcacgattctcaataattcccgtagcagctcttcagcaagtggctcctatggtcacttatctgcaagtgcaatcagccctgccttgagcttcacctactcttccgcgcccgtctctctccacatgcatcagcagatcctaagccgacaacagagcttaggttcagcctttggacacagccctccactcatccaccctgccccaacttttccaacacagaggcctattccagggatccctacggttctgaaccccgtccaggtcagctccggcccttctgagtcctcacagaacaagcccacgagtgagtctgcagtgagcagcactggtgacccgatgcacaacaagaggtccaagatcaaacccgatgaagacctccccagcccaggggctcgggggcagcaggaacagcccgaaggaacaacccttgtcaaggaggaaggggacaaagatgaaagcaaacaggagcctgaagtcatctatgagacaaactgccactgggaaggctgcgcgagggagttcgacacccaagagcagcttgtgcaccatataaataacgaccatattcatggagagaagaaggagttcgtgtgcaggtggctggactgctcaagagagcagaaacccttcaaagcccagtatatgttggtagtgcatatgagaagacacacgggcgagaagcctcacaaatgcacttttgaaggttgcacaaaggcctactcgagactagaaaacttgaaaacacacttgagatctcacactggagagaaaccatacgtctgtgagcacgaaggttgcaacaaggctttctcaaatgcctctgatcgcgccaaacaccaaaacagaacgcattccaatgagaaaccatatgtgtgcaaaatcccaggctgcactaagcgttacacagacccaagctccctccggaaacatgtgaagacagtgcatggcccagaggctcatgtcaccaagaagcagcgaggggacatccatcctcggccgccacccccgagagattccggcagccattcacagtccaggtcgcctggccgaccgactcagggagcccttggtgagcagcaggacctcagcaacactacctcaaagcgggaagaatgcctccaggtgaaaaccgtcaaggcagagaagccaatgacatctcagccaagccctggtggtcagtcttcatgcagcagccaacagtcccccatcagcaactattccaacagtgggctcgagcttcctctgaccgatggaggtagtataggagacctcagtgccatcgatgaaaccccaatcatggactcaaccatttccactgcaaccacagcccttgctttgcaagccaggagaaacccggcagggaccaaatggatggagcacgtaaaactagaaaggctaaaacaagtgaatggaatgtttccgcgactgaaccccattctaccccctaaagcccctgcggtctctcctctcataggaaatggcacacagtccaacaacacctgcagcttgggtgggcccatgacgcttctcccgggcagaagcgacctctctggggtggacgtcactatgctgaacatgctcaacagaagggacagcagcgccagcaccatcagctcggcctacctgagcagccgccgctcctcagggatctcgccctgcttctccagccgccgctccagcgaggcgtcacaggccgagggccggccgcagaacgtgagcgtggccgactcctacgaccccatctccaccgacgcctcgcgccgctccagcgaagccagccagagcgacggcctgcccagcctgctcagcctcacgcccgcccagcagtaccgcctcaaggccaagtacgcggctgccacaggagggccgccgccgacgcccctgcccaacatggagaggatgagcctgaagacgcgcctggcgctgctcggggatgccctcgagcctggcgtggccctgcctccagttcatgccccgaggaggtgcagcgacgggggagcccacggctacgggcggcgccacctgcagccgcacgatgcgccgggccacggcgtgaggagggccagcgacccggtgcggacaggctccgagggcctggccctgcctcgtgtgccgcgcttcagcagcctcagcagctgcaaccccccggcgatggccacgtccgcggagaagcgcagtctcgtgcttcagaattacacgcggcccgagggcggccagtcccgaaacttccactcgtccccctgtcctcccagcatcaccgagaacgtcaccctggagtccctgaccatggacgctgatgccaacctgaacgatgaggatttcctgccggacgacgtggtgcagtatttaaattcccagaaccaagcagggtacgagcagcacttccccagcgccctcccggacgacagcaaagtgccccacgggcccggtgactttgacgcgcccgggctgccagacagccacgctggccagcagttccatgccctcgagcagccctgccccgagggcagcaaaaccgacctgcccattcagtggaacgaagtcagctccggaagcgccgacctgtcctcctccaagctcaagtgtgggccgcggcccgctgtgccgcagactcgcgcctttgggttctgcaacggcatggtcgtccacccgcagaaccccttgaggagcgggcctgctgggggctatcagaccctcggggagaacagcaacccctacggtggcccagagcacttgatgctccacaacagccccggaagtggcaccagtggaaacgccttccatgaacagccctgtaaggccccgcagtatgggaactgtctcaacaggcagccagtggcccctggtgcactcgacggtgcctgtggtgccgggattcaagcctcaaagctgaagagcacccccatgcaagggagcgggggccagctgaatttcggcctgccggtagcgccaaatgagtcagctggcagcatggtgaatggcatgcagaaccaggacccagtgggacaggggtacctggctcaccagctcctcggcgacagcatgcagcacccgggggcaggccgccccggtcagcagatgcttgggcagattagtgctacctcacacatcaacatctaccaagggccagagagctgcctgccaggggctcacggcatgggcagccagccgtcaagcttggcagttgtcaggggctaccagccatgtgccagctttgggggcagcaggcgccaggctatgccgagggacagccttgctctgcagtcaggacagctcagtgacacaagtcagacctgcagggtgaatggtatcaagatggagatgaaagggcagccccatccgctgtgctctaatctgcagaattactctggtcagttctatgaccaaaccgtgggcttcagtcagcaagacacgaaagctggttcattctctatttcagacgccagctgcctgctacaggggaccagcgccaaaaactctgagttactttccccaggtgctaatcaggtgacaagcacagtggacagcctcgacagccatgacctggaaggggtacagattgacttcgatgccatcatagacgatggggaccactccagcctgatgtcgggggccctgagcccaagtatcattcagaacctttcccatagctcctcccgcctcaccacgcctcgggcgtccctcccattcccagcgctgtccatgagcaccaccaacatggctatcggggacatgagttctttgctgacctccctagcggaagaaagcaaattccttgcagttatgcaataggctttaggaaaaaaagactgcaaccaacggaaatcaataggagttgaagagattaaactgactttgttttggctgtttttttagttctgtatgtattttagcaatctcatctcacctaactgagatgtgtttcaattatattccttttatggaaaaggactctgaaaaaccctaaagtattctagggagaaactgtcttccatttcagttttgaatcagtattgttacactcaaaccaccctctttttaaaaaaaaaaaaaaaaactgtaagccccgccccctttttagtaaaccgatgtaaatttgtgatgtgcatattcttctttcttttagaagagcagtcaaattaaaggatttgacatgttttgctgttgctcaaaggaaataggagttggtgtgcttgtgaccaaggggttacacttccagcttttaaaattctcctttacatgtgctcagtgttttgttttgtgttttggtttctgttttttattttaattcccacattgggcacaagaatcagaatatggatagctagtttaagaaacttttgtgggtgcactgtagcatagatgacagaatattgatgttccccccatctccaattcagttcagggcattccacagttaaacagaaatgggaacgtggggctcttataaatgaaatgggcgctcacagttttggttttcagctcttcatgtctgtaagtgtgctttgggggaggctatgtctgtatggtcgattctcagttatcacatttgcctctcctcccactaccttcatgaacattcagtgctgtttcgcactgcagttagagagaagggacggacagttggtgacactcagccacattgctacttttatctgttctggtaagaagttagatagatggtagattgaagcaattgggtagaattagttgggggaatatttatgagttgctgtgtttgttgattagttccatctctttcccattttaactgagaattgattatatatagctctaagtatataggtatttaaacaaccccacaagcggctgtatcagtaacatttattaattccactatagtgagggaggatttccattctaaataccttattttgagggatttataaaacttagttgtaaaagagaaagcccacatagtgggaataaattgcttcagccatttttagtatttgagagcactagggaagatgtttagtagctgtgtggatgccttttttcacaccctgtctattgaatgctgcatccattcacgaagttaaatgttacatgcagttagtccttaatgtggactggatctgtacttttgttttggattaaaacatttaaagatttttgaagtgcagctactccccacgtgcatttgatacacataaaagtcatactgtgtgtgcacaaagagtacatggattttccagcatattgctttaaaaaattatataaactgttaaaatattaacacctcaggctacctgctgtattctgtcccattgacccctggaattggatttactgcaagtgattgataattcaattatgtggcttttcccctttaatcttgccatttaaattacagtagaaagacaaaatcaagtaaaataaagtgttagataatagaaagagtgttaagaccagcccacttttctcatgtttatgttctttcatttggaccaagaatctccgcatggaggttgatttgccactggggactttggctaagactattaggtttgctttcaactagatgttcctgagacaagcagagggacactgcaattccccttccatgcctgctgttctcccccatgtaagtcttctttgaaattaacggatgtgtctcctttggaacagccccataacaaaagagaactactgatctgagcataggaaagtagaggctctaccacttttcagttgaaaaagcaagactttctctgtgtttctgaaacaaggcataatgttgtcacagaatcagagatccagtctcacttttccacaaatctccaaatctccagtcttatcttgtgtgctctaatggtttggttcaatccctttccaactcttgttttcaaagcatggggcctgagtgttctccactcctcctaagaaaggagcttgggtggaagggaccatgctgacctcctccatcagagggctcttccagtagtattctcggatgcaacctccatttctcagttaccattatttcctgtatcagctttgtccttcctggagggatgcacagtgatccggcccaccactgttgttgtcttgtgcttctgctctttcctatggtttcaggttattttctgggtttcccctattcttcttttatttctttttttttttatatttgctttcctttctactgcttttagatttgcaggagatgcaagtttcagctcaatgtttggcttctctcaatatggaaatttcagaaggacagaggagaggagggaggaagaagaaagtatactcctccagaatttcagtgatctgttgtggcagtccagtggaaggaaggtcttttgaggtcacttagaagcatctttttgggacatccttttgggatctctgtaggctaggcatctcatatcttgagactcacccccagcctccaagcctctctccatttctctaacctatgcattttagagcgagaggaccgcctcactagtgtcaccatcctgccttttctaaaacatgcaggctcacacattctactcctgcttaatgtctgtgttaaacgttttctaaccatttttgttttatttttctgaaaaagttaacccctcccaactcctcacacattggctcttcctcttgagccacaaagttttgattcttgcgatgtatgtgccttattttatgttaatcttgtcaatgagagggaccagttggtgttgcccaatcagcactccaaggctgtgtgtgcaccagccagagagcgcacggtggtagcagagtcgaggctgtcttgtatcctggtttcatgtgttgttttgaactgataggaggatgttctcttctgacaagttacccttgtgtatcctgcagacatgtaaaataaaatacaagttcatttttttcaccttttttagatttttttaaaaaataaaatgtgtaatcctttttttaaaagaaacacatgtaaatacatttaagtattgtaggcatagcgttcagatgtgactggcccaggcgttcctcggacaagcctgcattccccgtgatcacgcccacctcaagcccaggggctgcagcccagccacagatgaactctacctttgctttcagaaccacttagtccttttgtaacaaagaaaaaaaaatgtttcttacaatgtcaataaaaaattctttgtatggaaa